SEQUENCE LISTING (74 sequences)

SEQ ID NO: 1 - DNA, 1866 bp, synthetic construct
  source 1..1866: mol_type = other DNA
atgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggctcttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataa

SEQ ID NO: 2 - DNA, 1866 bp, synthetic construct
  source 1..1866: mol_type = other DNA
acgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggctcttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataa

SEQ ID NO: 3 - DNA, 2211 bp, synthetic construct
  source 1..2211: mol_type = other DNA
atggctgccgatggttatcttccagattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaagcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 4 - DNA, 121 bp, synthetic construct
  source 1..121: mol_type = other DNA
gaggtcctgtattagaggtcacgtgagtgttttgcgacattttgcgacaccatgtggtcacgctgggtatttaagcccgagtgagcacgcagggtctccattttgaagcgggaggtttgaa

SEQ ID NO: 5 - DNA, 43 bp, synthetic construct
  source 1..43: mol_type = other DNA
agggtctccattttgaagcgggaggtttgaacgcgcagccgcc

SEQ ID NO: 6 - DNA, 37 bp, synthetic construct
  source 1..37: mol_type = other DNA
gcccgagtgagcacgcaggtttgaacgcgcagccgcc

SEQ ID NO: 7 - DNA, 59 bp, synthetic construct
  source 1..59: mol_type = other DNA
gcccgagtgagcacgcagggtctccattttgaagcgggaggtttgaacgcgcagccgcc

SEQ ID NO: 8 - DNA, 66 bp, synthetic construct
  source 1..66: mol_type = other DNA
tatttaagcccgagtgagcacgcagggtctccattttgaagcgggaggtttgaacgcgcagccgcc

SEQ ID NO: 9 - DNA, 119 bp, synthetic construct
  source 1..119: mol_type = other DNA
gaggtcctgtattagatttgcgacattttgcgacaccatgtggtcacgctgggtatttaagcccgagtgagcacgcagggtctccattttgaagcgggaggtttgaacgcgcagccgcc

SEQ ID NO: 10 - DNA, 113 bp, synthetic construct
  source 1..113: mol_type = other DNA
gaggtcctgtattagaggtcacgtgagtgtcatgtggtcacgctgggtatttaagcccgagtgagcacgcagggtctccattttgaagcgggaggtttgaacgcgcagccgcc

SEQ ID NO: 11 - DNA, 99 bp, synthetic construct
  source 1..99: mol_type = other DNA
gaggtcctgtattagacatgtggtcacgctgggtatttaagcccgagtgagcacgcagggtctccattttgaagcgggaggtttgaacgcgcagccgcc

SEQ ID NO: 12 - DNA, 112 bp, synthetic construct
  source 1..112: mol_type = other DNA
gaggtcctgtattagatttgcgacattttgcgacaccatgtggtcacgctggggcccgagtgagcacgcagggtctccattttgaagcgggaggtttgaacgcgcagccgcc

SEQ ID NO: 13 - DNA, 106 bp, synthetic construct
  source 1..106: mol_type = other DNA
gaggtcctgtattagaggtcacgtgagtgtcatgtggtcacgctggggcccgagtgagcacgcagggtctccattttgaagcgggaggtttgaacgcgcagccgcc

SEQ ID NO: 14 - DNA, 92 bp, synthetic construct
  source 1..92: mol_type = other DNA
gaggtcctgtattagacatgtggtcacgctggggcccgagtgagcacgcagggtctccattttgaagcgggaggtttgaacgcgcagccgcc

SEQ ID NO: 15 - DNA, 75 bp, synthetic construct
  source 1..75: mol_type = other DNA
gaggtcctgtattagagcccgagtgagcacgcagggtctccattttgaagcgggaggtttgaacgcgcagccgcc

SEQ ID NO: 16 - DNA, 233 bp, synthetic construct
  source 1..233: mol_type = other DNA
cgcgcagccgccaagccgaattctgcagatccggttaattcgtttcagttgaactttggtctcctgtccttcttatcttatcggttaccatagaaactggttacttattaactgcttggtgcgcttcgcgataaaagacttacgtcatcgggttacccctagtgatggagcggccgctttcagttgaactttggtctctgcgtatttctttcttatctagtttccatgctcta

SEQ ID NO: 17 - DNA, 69 bp, synthetic construct
  source 1..69: mol_type = other DNA
cgcgcagccgccaagccgtttcagttgaactttggtctctgcgtatttctttcttatctagtttccatg

SEQ ID NO: 18 - DNA, 100 bp, synthetic construct
  source 1..100: mol_type = other DNA
gtcttacccccaataaggggatttgctctatttaattaggaataaggtcgattactgatagaacaaatccaggctactgtgtttagtaatcagatttgtt

SEQ ID NO: 19 - DNA, 200 bp, synthetic construct
  source 1..200: mol_type = other DNA
gtcttacccccaataaggggatttgctctatttaattaggaataaggtcgattactgatagaacaaatccaggctactgtgtttagtaatcagatttgttcgtgaccgatatgcacgggcaaaacggcaggaggttgttagcgcgacctcctgccacccgctttcacgaaggtcatgtgtaaaaggccgcagcgtaacta

SEQ ID NO: 20 - DNA, 300 bp, synthetic construct
  source 1..300: mol_type = other DNA
gtcttacccccaataaggggatttgctctatttaattaggaataaggtcgattactgatagaacaaatccaggctactgtgtttagtaatcagatttgttcgtgaccgatatgcacgggcaaaacggcaggaggttgttagcgcgacctcctgccacccgctttcacgaaggtcatgtgtaaaaggccgcagcgtaactattactaatgaattcaggacagacagtggctacggctcagtttgggttgtgctgttgctgggcggcgatgacgcctgtacgcatttggtgatccggttctg

SEQ ID NO: 21 - DNA, 100 bp, synthetic construct
  source 1..100: mol_type = other DNA
catacagacgaaactagttggagattatcgagcattctatcacgtcgacgaccactagtgagttactagagccgaggggtaacgttgatgctcctaaaaa

SEQ ID NO: 22 - DNA, 200 bp, synthetic construct
  source 1..200: mol_type = other DNA
catacagacgaaactagttggagattatcgagcattctatcacgtcgacgaccactagtgagttactagagccgaggggtaacgttgatgctcctaaaaatctctcggtcgatgcaagcgattacactcctgtcacatcataatcgtttgctattcagggcttgatcaacactggattgcttttcacttaaagtattatg

SEQ ID NO: 23 - DNA, 300 bp, synthetic construct
  source 1..300: mol_type = other DNA
catacagacgaaactagttggagattatcgagcattctatcacgtcgacgaccactagtgagttactagagccgaggggtaacgttgatgctcctaaaaatctctcggtcgatgcaagcgattacactcctgtcacatcataatcgtttgctattcagggcttgatcaacactggattgcttttcacttaaagtattatgcacgacagggtacgtgtaccatataaacctgttataacttacctcagactagttggaaatgtggctagatcttagcttacgttactaaagggtccacgtt

SEQ ID NO: 24 - DNA, 7443 bp, synthetic construct
  source 1..7443: mol_type = other DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgacgcgtattgggatgaggtcctgtattagaggtcacgtgagtgttttgcgacattttgcgacaccatgtggtcacgctgggtatttaagcccgagtgagcacgcagggtctccattttgaagcgggaggtttgaacgcgcagccgccatgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggctcttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatggctgccgatggttatcttccagattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaagcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaattgcttgttaatcaataaaccgtttaattcgtttcagttgaactttggtctctgcgaagggcgaattcgtttaaacctgcaggactagaggtcctgtattagaggtcacgtgagtgttttgcgacattttgcgacaccatgtggtcacgctgggtatttaagcccgagtgagcacgcagggtctccattttgaagcgggaggtttgaacgcgcagccgccaagccgaattctgcagatccggttaattcgtttcagttgaactttggtctcctgtccttcttatcttatcggttaccatagaaactggttacttattaactgcttggtgcgcttcgcgataaaagacttacgtcatcgggttacccctagtgatggagcggccgctttcagttgaactttggtctctgcgtatttctttcttatctagtttccatgctctagaggtcctgtattagaggtcacgtgagtgttttgcgacattttgcgacaccatgtggtcacgctgggtatttaagcccgagtgagcacgcagggtctccattttgaagcgggaggtttgaacgcgcagccgccaagccgaattctgcagaatcccaatggcgcgccgagcttggctcgagcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccagggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 25 - DNA, 7443 bp, synthetic construct
  source 1..7443: mol_type = other DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgacgcgtattgggatgaggtcctgtattagaggtcacgtgagtgttttgcgacattttgcgacaccatgtggtcacgctgggtatttaagcccgagtgagcacgcagggtctccattttgaagcgggaggtttgaacgcgcagccgccacgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggctcttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatggctgccgatggttatcttccagattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaagcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaattgcttgttaatcaataaaccgtttaattcgtttcagttgaactttggtctctgcgaagggcgaattcgtttaaacctgcaggactagaggtcctgtattagaggtcacgtgagtgttttgcgacattttgcgacaccatgtggtcacgctgggtatttaagcccgagtgagcacgcagggtctccattttgaagcgggaggtttgaacgcgcagccgccaagccgaattctgcagatccggttaattcgtttcagttgaactttggtctcctgtccttcttatcttatcggttaccatagaaactggttacttattaactgcttggtgcgcttcgcgataaaagacttacgtcatcgggttacccctagtgatggagcggccgctttcagttgaactttggtctctgcgtatttctttcttatctagtttccatgctctagaggtcctgtattagaggtcacgtgagtgttttgcgacattttgcgacaccatgtggtcacgctgggtatttaagcccgagtgagcacgcagggtctccattttgaagcgggaggtttgaacgcgcagccgccaagccgaattctgcagaatcccaatggcgcgccgagcttggctcgagcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccagggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 26 - DNA, 7322 bp, synthetic construct
  source 1..7322: mol_type = other DNA
atgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggctcttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatggctgccgatggttatcttccagattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaagcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaattgcttgttaatcaataaaccgtttaattcgtttcagttgaactttggtctctgcgaagggcgaattcgtttaaacctgcaggactagaggtcctgtattagaggtcacgtgagtgttttgcgacattttgcgacaccatgtggtcacgctgggtatttaagcccgagtgagcacgcagggtctccattttgaagcgggaggtttgaacgcgcagccgccaagccgaattctgcagatccggttaattcgtttcagttgaactttggtctcctgtccttcttatcttatcggttaccatagaaactggttacttattaactgcttggtgcgcttcgcgataaaagacttacgtcatcgggttacccctagtgatggagcggccgctttcagttgaactttggtctctgcgtatttctttcttatctagtttccatgctctagaggtcctgtattagaggtcacgtgagtgttttgcgacattttgcgacaccatgtggtcacgctgggtatttaagcccgagtgagcacgcagggtctccattttgaagcgggaggtttgaacgcgcagccgccaagccgaattctgcagaatcccaatggcgcgccgagcttggctcgagcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccagggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgacgcgtattgggatcgcgcagccgcc

SEQ ID NO: 27 - DNA, 7322 bp, synthetic construct
  source 1..7322: mol_type = other DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgacgcgtattgggatcgcgcagccgccacgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggctcttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatggctgccgatggttatcttccagattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaagcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaattgcttgttaatcaataaaccgtttaattcgtttcagttgaactttggtctctgcgaagggcgaattcgtttaaacctgcaggactagaggtcctgtattagaggtcacgtgagtgttttgcgacattttgcgacaccatgtggtcacgctgggtatttaagcccgagtgagcacgcagggtctccattttgaagcgggaggtttgaacgcgcagccgccaagccgaattctgcagatccggttaattcgtttcagttgaactttggtctcctgtccttcttatcttatcggttaccatagaaactggttacttattaactgcttggtgcgcttcgcgataaaagacttacgtcatcgggttacccctagtgatggagcggccgctttcagttgaactttggtctctgcgtatttctttcttatctagtttccatgctctagaggtcctgtattagaggtcacgtgagtgttttgcgacattttgcgacaccatgtggtcacgctgggtatttaagcccgagtgagcacgcagggtctccattttgaagcgggaggtttgaacgcgcagccgccaagccgaattctgcagaatcccaatggcgcgccgagcttggctcgagcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccagggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 28 - DNA, 7104 bp, synthetic construct
  source 1..7104: mol_type = other DNA
atgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggctcttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatggctgccgatggttatcttccagattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaagcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaattgcttgttaatcaataaaccgtttaattcgtttcagttgaactttggtctctgccctaggggaggtcctgtattagaggtcacgtgagtgttttgcgacattttgcgacaccatgtggtcacgctgggtatttaagcccgagtgagcacgcagggtctccattttgaagcgggaggtttgaacgcgcagccgccaagccgtttcagttgaactttggtctctgcgtatttctttcttatctagtttccatggaggtcctgtattagaggtcacgtgagtgttttgcgacattttgcgacaccatgtggtcacgctgggtatttaagcccgagtgagcacgcagggtctccattttgaagcgggaggtttgaaatcccaatggcgcgccgagcttggctcgagcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccagggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgacgcgtattgggatcgcgcagccgcc

SEQ ID NO: 29 - DNA, 7135 bp, synthetic construct
  source 1..7135: mol_type = other DNA
atgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggctcttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatggctgccgatggttatcttccagattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaagcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaattgcttgttaatcaataaaccgtttaattcgtttcagttgaactttggtctctgccctaggggaggtcctgtattagaggtcacgtgagtgttttgcgacattttgcgacaccatgtggtcacgctgggtatttaagcccgagtgagcacgcagggtctccattttgaagcgggaggtttgaagtcttacccccaataaggggatttgctctatttaattaggaataaggtcgattactgatagaacaaatccaggctactgtgtttagtaatcagatttgttgaggtcctgtattagaggtcacgtgagtgttttgcgacattttgcgacaccatgtggtcacgctgggtatttaagcccgagtgagcacgcagggtctccattttgaagcgggaggtttgaaatcccaatggcgcgccgagcttggctcgagcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccagggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgacgcgtattgggatcgcgcagccgcc

SEQ ID NO: 30 - DNA, 7235 bp, synthetic construct
  source 1..7235: mol_type = other DNA
atgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggctcttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatggctgccgatggttatcttccagattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaagcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaattgcttgttaatcaataaaccgtttaattcgtttcagttgaactttggtctctgccctaggggaggtcctgtattagaggtcacgtgagtgttttgcgacattttgcgacaccatgtggtcacgctgggtatttaagcccgagtgagcacgcagggtctccattttgaagcgggaggtttgaagtcttacccccaataaggggatttgctctatttaattaggaataaggtcgattactgatagaacaaatccaggctactgtgtttagtaatcagatttgttcgtgaccgatatgcacgggcaaaacggcaggaggttgttagcgcgacctcctgccacccgctttcacgaaggtcatgtgtaaaaggccgcagcgtaactagaggtcctgtattagaggtcacgtgagtgttttgcgacattttgcgacaccatgtggtcacgctgggtatttaagcccgagtgagcacgcagggtctccattttgaagcgggaggtttgaaatcccaatggcgcgccgagcttggctcgagcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccagggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgacgcgtattgggatcgcgcagccgcc

SEQ ID NO: 31 - DNA, 7335 bp, synthetic construct
  source 1..7335: mol_type = other DNA
atgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggctcttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatggctgccgatggttatcttccagattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaagcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaattgcttgttaatcaataaaccgtttaattcgtttcagttgaactttggtctctgccctaggggaggtcctgtattagaggtcacgtgagtgttttgcgacattttgcgacaccatgtggtcacgctgggtatttaagcccgagtgagcacgcagggtctccattttgaagcgggaggtttgaagtcttacccccaataaggggatttgctctatttaattaggaataaggtcgattactgatagaacaaatccaggctactgtgtttagtaatcagatttgttcgtgaccgatatgcacgggcaaaacggcaggaggttgttagcgcgacctcctgccacccgctttcacgaaggtcatgtgtaaaaggccgcagcgtaactattactaatgaattcaggacagacagtggctacggctcagtttgggttgtgctgttgctgggcggcgatgacgcctgtacgcatttggtgatccggttctggaggtcctgtattagaggtcacgtgagtgttttgcgacattttgcgacaccatgtggtcacgctgggtatttaagcccgagtgagcacgcagggtctccattttgaagcgggaggtttgaaatcccaatggcgcgccgagcttggctcgagcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccagggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgacgcgtattgggatcgcgcagccgcc

SEQ ID NO: 32 - DNA, 7135 bp, synthetic construct
  source 1..7135: mol_type = other DNA
atgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggctcttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatggctgccgatggttatcttccagattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaagcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaattgcttgttaatcaataaaccgtttaattcgtttcagttgaactttggtctctgccctaggggaggtcctgtattagaggtcacgtgagtgttttgcgacattttgcgacaccatgtggtcacgctgggtatttaagcccgagtgagcacgcagggtctccattttgaagcgggaggtttgaacatacagacgaaactagttggagattatcgagcattctatcacgtcgacgaccactagtgagttactagagccgaggggtaacgttgatgctcctaaaaagaggtcctgtattagaggtcacgtgagtgttttgcgacattttgcgacaccatgtggtcacgctgggtatttaagcccgagtgagcacgcagggtctccattttgaagcgggaggtttgaaatcccaatggcgcgccgagcttggctcgagcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccagggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgacgcgtattgggatcgcgcagccgcc

SEQ ID NO: 33 - DNA, 7235 bp, synthetic construct
  source 1..7235: mol_type = other DNA
atgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggctcttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatggctgccgatggttatcttccagattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaagcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaattgcttgttaatcaataaaccgtttaattcgtttcagttgaactttggtctctgccctaggggaggtcctgtattagaggtcacgtgagtgttttgcgacattttgcgacaccatgtggtcacgctgggtatttaagcccgagtgagcacgcagggtctccattttgaagcgggaggtttgaacatacagacgaaactagttggagattatcgagcattctatcacgtcgacgaccactagtgagttactagagccgaggggtaacgttgatgctcctaaaaatctctcggtcgatgcaagcgattacactcctgtcacatcataatcgtttgctattcagggcttgatcaacactggattgcttttcacttaaagtattatggaggtcctgtattagaggtcacgtgagtgttttgcgacattttgcgacaccatgtggtcacgctgggtatttaagcccgagtgagcacgcagggtctccattttgaagcgggaggtttgaaatcccaatggcgcgccgagcttggctcgagcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccagggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgacgcgtattgggatcgcgcagccgcc

SEQ ID NO: 34 - DNA, 7335 bp, synthetic construct
  source 1..7335: mol_type = other DNA
atgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggctcttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatggctgccgatggttatcttccagattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaagcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaattgcttgttaatcaataaaccgtttaattcgtttcagttgaactttggtctctgccctaggggaggtcctgtattagaggtcacgtgagtgttttgcgacattttgcgacaccatgtggtcacgctgggtatttaagcccgagtgagcacgcagggtctccattttgaagcgggaggtttgaacatacagacgaaactagttggagattatcgagcattctatcacgtcgacgaccactagtgagttactagagccgaggggtaacgttgatgctcctaaaaatctctcggtcgatgcaagcgattacactcctgtcacatcataatcgtttgctattcagggcttgatcaacactggattgcttttcacttaaagtattatgcacgacagggtacgtgtaccatataaacctgttataacttacctcagactagttggaaatgtggctagatcttagcttacgttactaaagggtccacgttgaggtcctgtattagaggtcacgtgagtgttttgcgacattttgcgacaccatgtggtcacgctgggtatttaagcccgagtgagcacgcagggtctccattttgaagcgggaggtttgaaatcccaatggcgcgccgagcttggctcgagcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccagggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgacgcgtattgggatcgcgcagccgcc

SEQ ID NO: 35 - DNA, 7294 bp, synthetic construct
  source 1..7294: mol_type = other DNA
atgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggctcttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatggctgccgatggttatcttccagattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaagcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaattgcttgttaatcaataaaccgtttaattcgtttcagttgaactttggtctctgccctaggggaggtcctgtattagaggtcacgtgagtgttttgcgacattttgcgacaccatgtggtcacgctgggtatttaagcccgagtgagcacgcagggtctccattttgaagcgggaggtttgaacgcgcagccgccaagccgtttcagttgaactttggtctctgcgtatttctttcttatctagtttccatggaggtcctgtattagaggtcacgtgagtgttttgcgacattttgcgacaccatgtggtcacgctgggtatttaagcccgagtgagcacgcagggtctccattttgaagcgggaggtttgaacgcgcagccgccaagccgtttcagttgaactttggtctctgcgtatttctttcttatctagtttccatggaggtcctgtattagaggtcacgtgagtgttttgcgacattttgcgacaccatgtggtcacgctgggtatttaagcccgagtgagcacgcagggtctccattttgaagcgggaggtttgaaatcccaatggcgcgccgagcttggctcgagcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccagggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgacgcgtattgggatcgcgcagccgcc

SEQ ID NO: 36 - DNA, 6939 bp, synthetic construct
  source 1..6939: mol_type = other DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgacgcgtattgggatagggtctccattttgaagcgggaggtttgaacgcgcagccgccatgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggctcttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatggctgccgatggttatcttccagattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaagcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaattgcttgttaatcaataaaccgtttaattcgtttcagttgaactttggtctctgcggaggtcctgtattagaggtcacgtgagtgttttgcgacattttgcgacaccatgtggtcacgctgggtatttaagcccgagtgagcacgcagggtctccattttgaagcgggaggtttgaaatcccaatggcgcgccgagcttggctcgagcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccagggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 37 - DNA, 6933 bp, synthetic construct
  source 1..6933: mol_type = other DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgacgcgtattgggatgcccgagtgagcacgcaggtttgaacgcgcagccgccatgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggctcttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatggctgccgatggttatcttccagattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaagcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaattgcttgttaatcaataaaccgtttaattcgtttcagttgaactttggtctctgcggaggtcctgtattagaggtcacgtgagtgttttgcgacattttgcgacaccatgtggtcacgctgggtatttaagcccgagtgagcacgcagggtctccattttgaagcgggaggtttgaaatcccaatggcgcgccgagcttggctcgagcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccagggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 38 - DNA, 6955 bp, synthetic construct
  source 1..6955: mol_type = other DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgacgcgtattgggatgcccgagtgagcacgcagggtctccattttgaagcgggaggtttgaacgcgcagccgccatgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggctcttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatggctgccgatggttatcttccagattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaagcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaattgcttgttaatcaataaaccgtttaattcgtttcagttgaactttggtctctgcggaggtcctgtattagaggtcacgtgagtgttttgcgacattttgcgacaccatgtggtcacgctgggtatttaagcccgagtgagcacgcagggtctccattttgaagcgggaggtttgaaatcccaatggcgcgccgagcttggctcgagcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccagggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 39 - DNA, 6962 bp, synthetic construct
  source 1..6962: mol_type = other DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgacgcgtattgggattatttaagcccgagtgagcacgcagggtctccattttgaagcgggaggtttgaacgcgcagccgccatgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggctcttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatggctgccgatggttatcttccagattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaagcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaattgcttgttaatcaataaaccgtttaattcgtttcagttgaactttggtctctgcggaggtcctgtattagaggtcacgtgagtgttttgcgacattttgcgacaccatgtggtcacgctgggtatttaagcccgagtgagcacgcagggtctccattttgaagcgggaggtttgaaatcccaatggcgcgccgagcttggctcgagcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccagggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 40 - DNA, 7015 bp, synthetic construct
  source 1..7015: mol_type = other DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgacgcgtattgggatgaggtcctgtattagatttgcgacattttgcgacaccatgtggtcacgctgggtatttaagcccgagtgagcacgcagggtctccattttgaagcgggaggtttgaacgcgcagccgccatgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggctcttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatggctgccgatggttatcttccagattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaagcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaattgcttgttaatcaataaaccgtttaattcgtttcagttgaactttggtctctgcggaggtcctgtattagaggtcacgtgagtgttttgcgacattttgcgacaccatgtggtcacgctgggtatttaagcccgagtgagcacgcagggtctccattttgaagcgggaggtttgaaatcccaatggcgcgccgagcttggctcgagcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccagggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 41 - DNA, 7009 bp, synthetic construct
  source 1..7009: mol_type = other DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgacgcgtattgggatgaggtcctgtattagaggtcacgtgagtgtcatgtggtcacgctgggtatttaagcccgagtgagcacgcagggtctccattttgaagcgggaggtttgaacgcgcagccgccatgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggctcttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatggctgccgatggttatcttccagattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaagcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaattgcttgttaatcaataaaccgtttaattcgtttcagttgaactttggtctctgcggaggtcctgtattagaggtcacgtgagtgttttgcgacattttgcgacaccatgtggtcacgctgggtatttaagcccgagtgagcacgcagggtctccattttgaagcgggaggtttgaaatcccaatggcgcgccgagcttggctcgagcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccagggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 42 - DNA, 6995 bp, synthetic construct
  source 1..6995: mol_type = other DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgacgcgtattgggatgaggtcctgtattagacatgtggtcacgctgggtatttaagcccgagtgagcacgcagggtctccattttgaagcgggaggtttgaacgcgcagccgccatgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggctcttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatggctgccgatggttatcttccagattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaagcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaattgcttgttaatcaataaaccgtttaattcgtttcagttgaactttggtctctgcggaggtcctgtattagaggtcacgtgagtgttttgcgacattttgcgacaccatgtggtcacgctgggtatttaagcccgagtgagcacgcagggtctccattttgaagcgggaggtttgaaatcccaatggcgcgccgagcttggctcgagcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccagggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 43 - DNA, 7008 bp, synthetic construct
  source 1..7008: mol_type = other DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgacgcgtattgggatgaggtcctgtattagatttgcgacattttgcgacaccatgtggtcacgctggggcccgagtgagcacgcagggtctccattttgaagcgggaggtttgaacgcgcagccgccatgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggctcttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatggctgccgatggttatcttccagattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaagcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaattgcttgttaatcaataaaccgtttaattcgtttcagttgaactttggtctctgcggaggtcctgtattagaggtcacgtgagtgttttgcgacattttgcgacaccatgtggtcacgctgggtatttaagcccgagtgagcacgcagggtctccattttgaagcgggaggtttgaaatcccaatggcgcgccgagcttggctcgagcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccagggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 44 - DNA, 7002 bp, synthetic construct
  source 1..7002: mol_type = other DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgacgcgtattgggatgaggtcctgtattagaggtcacgtgagtgtcatgtggtcacgctggggcccgagtgagcacgcagggtctccattttgaagcgggaggtttgaacgcgcagccgccatgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggctcttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatggctgccgatggttatcttccagattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaagcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaattgcttgttaatcaataaaccgtttaattcgtttcagttgaactttggtctctgcggaggtcctgtattagaggtcacgtgagtgttttgcgacattttgcgacaccatgtggtcacgctgggtatttaagcccgagtgagcacgcagggtctccattttgaagcgggaggtttgaaatcccaatggcgcgccgagcttggctcgagcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccagggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 45 - DNA, 6988 bp, synthetic construct
  source 1..6988: mol_type = other DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgacgcgtattgggatgaggtcctgtattagacatgtggtcacgctggggcccgagtgagcacgcagggtctccattttgaagcgggaggtttgaacgcgcagccgccatgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggctcttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatggctgccgatggttatcttccagattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaagcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaattgcttgttaatcaataaaccgtttaattcgtttcagttgaactttggtctctgcggaggtcctgtattagaggtcacgtgagtgttttgcgacattttgcgacaccatgtggtcacgctgggtatttaagcccgagtgagcacgcagggtctccattttgaagcgggaggtttgaaatcccaatggcgcgccgagcttggctcgagcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccagggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 46 - DNA, 6971 bp, synthetic construct
  source 1..6971: mol_type = other DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgacgcgtattgggatgaggtcctgtattagagcccgagtgagcacgcagggtctccattttgaagcgggaggtttgaacgcgcagccgccatgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggctcttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatggctgccgatggttatcttccagattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaagcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaattgcttgttaatcaataaaccgtttaattcgtttcagttgaactttggtctctgcggaggtcctgtattagaggtcacgtgagtgttttgcgacattttgcgacaccatgtggtcacgctgggtatttaagcccgagtgagcacgcagggtctccattttgaagcgggaggtttgaaatcccaatggcgcgccgagcttggctcgagcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccagggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 47 - DNA, 6908 bp, synthetic construct
  source 1..6908: mol_type = other DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgacgcgtattgggatcgcgcagccgccatgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggctcttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatggctgccgatggttatcttccagattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaagcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaattgcttgttaatcaataaaccgtttaattcgtttcagttgaactttggtctctgcggaggtcctgtattagaggtcacgtgagtgttttgcgacattttgcgacaccatgtggtcacgctgggtatttaagcccgagtgagcacgcagggtctccattttgaagcgggaggtttgaaatcccaatggcgcgccgagcttggctcgagcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccagggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 48 - DNA, 7029 bp, synthetic construct
  source 1..7029: mol_type = other DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgacgcgtattgggatgaggtcctgtattagaggtcacgtgagtgttttgcgacattttgcgacaccatgtggtcacgctgggtatttaagcccgagtgagcacgcagggtctccattttgaagcgggaggtttgaacgcgcagccgccacgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggctcttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatggctgccgatggttatcttccagattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaagcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaattgcttgttaatcaataaaccgtttaattcgtttcagttgaactttggtctctgcggaggtcctgtattagaggtcacgtgagtgttttgcgacattttgcgacaccatgtggtcacgctgggtatttaagcccgagtgagcacgcagggtctccattttgaagcgggaggtttgaaatcccaatggcgcgccgagcttggctcgagcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccagggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 49 - DNA, 41 bp, synthetic construct
  source 1..41: mol_type = other DNA
ttgcttgttaatcaataaaccgtttaattcgtttcagttga

SEQ ID NO: 50 - DNA, 2208 bp, synthetic construct
  source 1..2208: mol_type = other DNA
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgtaa

SEQ ID NO: 51 - DNA, 2175 bp, synthetic construct
  source 1..2175: mol_type = other DNA
atgtcttttgttgatcaccctccagattggttggaagaagttggtgaaggtcttcgcgagtttttgggccttgaagcgggcccaccgaaaccaaaacccaatcagcagcatcaagatcaagcccgtggtcttgtgctgcctggttataactatctcggacccggaaacggtctcgatcgaggagagcctgtcaacagggcagacgaggtcgcgcgagagcacgacatctcgtacaacgagcagcttgaggcgggagacaacccctacctcaagtacaaccacgcggacgccgagtttcaggagaagctcgccgacgacacatccttcgggggaaacctcggaaaggcagtctttcaggccaagaaaagggttctcgaaccttttggcctggttgaagagggtgctaagacggcccctaccggaaagcggatagacgaccactttccaaaaagaaagaaggctcggaccgaagaggactccaagccttccacctcgtcagacgccgaagctggacccagcggatcccagcagctgcaaatcccagcccaaccagcctcaagtttgggagctgatacaatgtctgcgggaggtggcggcccattgggcgacaataaccaaggtgccgatggagtgggcaatgcctcgggagattggcattgcgattccacgtggatgggggacagagtcgtcaccaagtccacccgaacctgggtgctgcccagctacaacaaccaccagtaccgagagatcaaaagcggctccgtcgacggaagcaacgccaacgcctactttggatacagcaccccctgggggtactttgactttaaccgcttccacagccactggagcccccgagactggcaaagactcatcaacaactactggggcttcagaccccggtccctcagagtcaaaatcttcaacattcaagtcaaagaggtcacggtgcaggactccaccaccaccatcgccaacaacctcacctccaccgtccaagtgtttacggacgacgactaccagctgccctacgtcgtcggcaacgggaccgagggatgcctgccggccttccctccgcaggtctttacgctgccgcagtacggttacgcgacgctgaaccgcgacaacacagaaaatcccaccgagaggagcagcttcttctgcctagagtactttcccagcaagatgctgagaacgggcaacaactttgagtttacctacaactttgaggaggtgcccttccactccagcttcgctcccagtcagaacctgttcaagctggccaacccgctggtggaccagtacttgtaccgcttcgtgagcacaaataacactggcggagtccagttcaacaagaacctggccgggagatacgccaacacctacaaaaactggttcccggggcccatgggccgaacccagggctggaacctgggctccggggtcaaccgcgccagtgtcagcgccttcgccacgaccaataggatggagctcgagggcgcgagttaccaggtgcccccgcagccgaacggcatgaccaacaacctccagggcagcaacacctatgccctggagaacactatgatcttcaacagccagccggcgaacccgggcaccaccgccacgtacctcgagggcaacatgctcatcaccagcgagagcgagacgcagccggtgaaccgcgtggcgtacaacgtcggcgggcagatggccaccaacaaccagagctccaccactgcccccgcgaccggcacgtacaacctccaggaaatcgtgcccggcagcgtgtggatggagagggacgtgtacctccaaggacccatctgggccaagatcccagagacgggggcgcactttcacccctctccggccatgggcggattcggactcaaacacccaccgcccatgatgctcatcaagaacacgcctgtgcccggaaatatcaccagcttctcggacgtgcccgtcagcagcttcatcacccagtacagcaccgggcaggtcaccgtggagatggagtgggagctcaagaaggaaaactccaagaggtggaacccagagatccagtacacaaacaactacaacgacccccagtttgtggactttgccccggacagcaccggggaatacagaaccaccagacctatcggaacccgataccttacccgacccctttaa

SEQ ID NO: 52 - DNA, 7407 bp, synthetic construct
  source 1..7407: mol_type = other DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgacgcgtattgggatgaggtcctgtattagaggtcacgtgagtgttttgcgacattttgcgacaccatgtggtcacgctgggtatttaagcccgagtgagcacgcagggtctccattttgaagcgggaggtttgaacgcgcagccgccatgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggctcttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatgtcttttgttgatcaccctccagattggttggaagaagttggtgaaggtcttcgcgagtttttgggccttgaagcgggcccaccgaaaccaaaacccaatcagcagcatcaagatcaagcccgtggtcttgtgctgcctggttataactatctcggacccggaaacggtctcgatcgaggagagcctgtcaacagggcagacgaggtcgcgcgagagcacgacatctcgtacaacgagcagcttgaggcgggagacaacccctacctcaagtacaaccacgcggacgccgagtttcaggagaagctcgccgacgacacatccttcgggggaaacctcggaaaggcagtctttcaggccaagaaaagggttctcgaaccttttggcctggttgaagagggtgctaagacggcccctaccggaaagcggatagacgaccactttccaaaaagaaagaaggctcggaccgaagaggactccaagccttccacctcgtcagacgccgaagctggacccagcggatcccagcagctgcaaatcccagcccaaccagcctcaagtttgggagctgatacaatgtctgcgggaggtggcggcccattgggcgacaataaccaaggtgccgatggagtgggcaatgcctcgggagattggcattgcgattccacgtggatgggggacagagtcgtcaccaagtccacccgaacctgggtgctgcccagctacaacaaccaccagtaccgagagatcaaaagcggctccgtcgacggaagcaacgccaacgcctactttggatacagcaccccctgggggtactttgactttaaccgcttccacagccactggagcccccgagactggcaaagactcatcaacaactactggggcttcagaccccggtccctcagagtcaaaatcttcaacattcaagtcaaagaggtcacggtgcaggactccaccaccaccatcgccaacaacctcacctccaccgtccaagtgtttacggacgacgactaccagctgccctacgtcgtcggcaacgggaccgagggatgcctgccggccttccctccgcaggtctttacgctgccgcagtacggttacgcgacgctgaaccgcgacaacacagaaaatcccaccgagaggagcagcttcttctgcctagagtactttcccagcaagatgctgagaacgggcaacaactttgagtttacctacaactttgaggaggtgcccttccactccagcttcgctcccagtcagaacctgttcaagctggccaacccgctggtggaccagtacttgtaccgcttcgtgagcacaaataacactggcggagtccagttcaacaagaacctggccgggagatacgccaacacctacaaaaactggttcccggggcccatgggccgaacccagggctggaacctgggctccggggtcaaccgcgccagtgtcagcgccttcgccacgaccaataggatggagctcgagggcgcgagttaccaggtgcccccgcagccgaacggcatgaccaacaacctccagggcagcaacacctatgccctggagaacactatgatcttcaacagccagccggcgaacccgggcaccaccgccacgtacctcgagggcaacatgctcatcaccagcgagagcgagacgcagccggtgaaccgcgtggcgtacaacgtcggcgggcagatggccaccaacaaccagagctccaccactgcccccgcgaccggcacgtacaacctccaggaaatcgtgcccggcagcgtgtggatggagagggacgtgtacctccaaggacccatctgggccaagatcccagagacgggggcgcactttcacccctctccggccatgggcggattcggactcaaacacccaccgcccatgatgctcatcaagaacacgcctgtgcccggaaatatcaccagcttctcggacgtgcccgtcagcagcttcatcacccagtacagcaccgggcaggtcaccgtggagatggagtgggagctcaagaaggaaaactccaagaggtggaacccagagatccagtacacaaacaactacaacgacccccagtttgtggactttgccccggacagcaccggggaatacagaaccaccagacctatcggaacccgataccttacccgacccctttaattgcttgttaatcaataaaccgtttaattcgtttcagttgaactttggtctctgcgaagggcgaattcgtttaaacctgcaggactagaggtcctgtattagaggtcacgtgagtgttttgcgacattttgcgacaccatgtggtcacgctgggtatttaagcccgagtgagcacgcagggtctccattttgaagcgggaggtttgaacgcgcagccgccaagccgaattctgcagatccggttaattcgtttcagttgaactttggtctcctgtccttcttatcttatcggttaccatagaaactggttacttattaactgcttggtgcgcttcgcgataaaagacttacgtcatcgggttacccctagtgatggagcggccgctttcagttgaactttggtctctgcgtatttctttcttatctagtttccatgctctagaggtcctgtattagaggtcacgtgagtgttttgcgacattttgcgacaccatgtggtcacgctgggtatttaagcccgagtgagcacgcagggtctccattttgaagcgggaggtttgaacgcgcagccgccaagccgaattctgcagaatcccaatggcgcgccgagcttggctcgagcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccagggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 53 - DNA, 7407 bp, synthetic construct
  source 1..7407: mol_type = other DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgacgcgtattgggatgaggtcctgtattagaggtcacgtgagtgttttgcgacattttgcgacaccatgtggtcacgctgggtatttaagcccgagtgagcacgcagggtctccattttgaagcgggaggtttgaacgcgcagccgccacgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggctcttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatgtcttttgttgatcaccctccagattggttggaagaagttggtgaaggtcttcgcgagtttttgggccttgaagcgggcccaccgaaaccaaaacccaatcagcagcatcaagatcaagcccgtggtcttgtgctgcctggttataactatctcggacccggaaacggtctcgatcgaggagagcctgtcaacagggcagacgaggtcgcgcgagagcacgacatctcgtacaacgagcagcttgaggcgggagacaacccctacctcaagtacaaccacgcggacgccgagtttcaggagaagctcgccgacgacacatccttcgggggaaacctcggaaaggcagtctttcaggccaagaaaagggttctcgaaccttttggcctggttgaagagggtgctaagacggcccctaccggaaagcggatagacgaccactttccaaaaagaaagaaggctcggaccgaagaggactccaagccttccacctcgtcagacgccgaagctggacccagcggatcccagcagctgcaaatcccagcccaaccagcctcaagtttgggagctgatacaatgtctgcgggaggtggcggcccattgggcgacaataaccaaggtgccgatggagtgggcaatgcctcgggagattggcattgcgattccacgtggatgggggacagagtcgtcaccaagtccacccgaacctgggtgctgcccagctacaacaaccaccagtaccgagagatcaaaagcggctccgtcgacggaagcaacgccaacgcctactttggatacagcaccccctgggggtactttgactttaaccgcttccacagccactggagcccccgagactggcaaagactcatcaacaactactggggcttcagaccccggtccctcagagtcaaaatcttcaacattcaagtcaaagaggtcacggtgcaggactccaccaccaccatcgccaacaacctcacctccaccgtccaagtgtttacggacgacgactaccagctgccctacgtcgtcggcaacgggaccgagggatgcctgccggccttccctccgcaggtctttacgctgccgcagtacggttacgcgacgctgaaccgcgacaacacagaaaatcccaccgagaggagcagcttcttctgcctagagtactttcccagcaagatgctgagaacgggcaacaactttgagtttacctacaactttgaggaggtgcccttccactccagcttcgctcccagtcagaacctgttcaagctggccaacccgctggtggaccagtacttgtaccgcttcgtgagcacaaataacactggcggagtccagttcaacaagaacctggccgggagatacgccaacacctacaaaaactggttcccggggcccatgggccgaacccagggctggaacctgggctccggggtcaaccgcgccagtgtcagcgccttcgccacgaccaataggatggagctcgagggcgcgagttaccaggtgcccccgcagccgaacggcatgaccaacaacctccagggcagcaacacctatgccctggagaacactatgatcttcaacagccagccggcgaacccgggcaccaccgccacgtacctcgagggcaacatgctcatcaccagcgagagcgagacgcagccggtgaaccgcgtggcgtacaacgtcggcgggcagatggccaccaacaaccagagctccaccactgcccccgcgaccggcacgtacaacctccaggaaatcgtgcccggcagcgtgtggatggagagggacgtgtacctccaaggacccatctgggccaagatcccagagacgggggcgcactttcacccctctccggccatgggcggattcggactcaaacacccaccgcccatgatgctcatcaagaacacgcctgtgcccggaaatatcaccagcttctcggacgtgcccgtcagcagcttcatcacccagtacagcaccgggcaggtcaccgtggagatggagtgggagctcaagaaggaaaactccaagaggtggaacccagagatccagtacacaaacaactacaacgacccccagtttgtggactttgccccggacagcaccggggaatacagaaccaccagacctatcggaacccgataccttacccgacccctttaattgcttgttaatcaataaaccgtttaattcgtttcagttgaactttggtctctgcgaagggcgaattcgtttaaacctgcaggactagaggtcctgtattagaggtcacgtgagtgttttgcgacattttgcgacaccatgtggtcacgctgggtatttaagcccgagtgagcacgcagggtctccattttgaagcgggaggtttgaacgcgcagccgccaagccgaattctgcagatccggttaattcgtttcagttgaactttggtctcctgtccttcttatcttatcggttaccatagaaactggttacttattaactgcttggtgcgcttcgcgataaaagacttacgtcatcgggttacccctagtgatggagcggccgctttcagttgaactttggtctctgcgtatttctttcttatctagtttccatgctctagaggtcctgtattagaggtcacgtgagtgttttgcgacattttgcgacaccatgtggtcacgctgggtatttaagcccgagtgagcacgcagggtctccattttgaagcgggaggtttgaacgcgcagccgccaagccgaattctgcagaatcccaatggcgcgccgagcttggctcgagcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccagggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 54 - DNA, 7286 bp, synthetic construct
  source 1..7286: mol_type = other DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgacgcgtattgggatcgcgcagccgccatgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggctcttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatgtcttttgttgatcaccctccagattggttggaagaagttggtgaaggtcttcgcgagtttttgggccttgaagcgggcccaccgaaaccaaaacccaatcagcagcatcaagatcaagcccgtggtcttgtgctgcctggttataactatctcggacccggaaacggtctcgatcgaggagagcctgtcaacagggcagacgaggtcgcgcgagagcacgacatctcgtacaacgagcagcttgaggcgggagacaacccctacctcaagtacaaccacgcggacgccgagtttcaggagaagctcgccgacgacacatccttcgggggaaacctcggaaaggcagtctttcaggccaagaaaagggttctcgaaccttttggcctggttgaagagggtgctaagacggcccctaccggaaagcggatagacgaccactttccaaaaagaaagaaggctcggaccgaagaggactccaagccttccacctcgtcagacgccgaagctggacccagcggatcccagcagctgcaaatcccagcccaaccagcctcaagtttgggagctgatacaatgtctgcgggaggtggcggcccattgggcgacaataaccaaggtgccgatggagtgggcaatgcctcgggagattggcattgcgattccacgtggatgggggacagagtcgtcaccaagtccacccgaacctgggtgctgcccagctacaacaaccaccagtaccgagagatcaaaagcggctccgtcgacggaagcaacgccaacgcctactttggatacagcaccccctgggggtactttgactttaaccgcttccacagccactggagcccccgagactggcaaagactcatcaacaactactggggcttcagaccccggtccctcagagtcaaaatcttcaacattcaagtcaaagaggtcacggtgcaggactccaccaccaccatcgccaacaacctcacctccaccgtccaagtgtttacggacgacgactaccagctgccctacgtcgtcggcaacgggaccgagggatgcctgccggccttccctccgcaggtctttacgctgccgcagtacggttacgcgacgctgaaccgcgacaacacagaaaatcccaccgagaggagcagcttcttctgcctagagtactttcccagcaagatgctgagaacgggcaacaactttgagtttacctacaactttgaggaggtgcccttccactccagcttcgctcccagtcagaacctgttcaagctggccaacccgctggtggaccagtacttgtaccgcttcgtgagcacaaataacactggcggagtccagttcaacaagaacctggccgggagatacgccaacacctacaaaaactggttcccggggcccatgggccgaacccagggctggaacctgggctccggggtcaaccgcgccagtgtcagcgccttcgccacgaccaataggatggagctcgagggcgcgagttaccaggtgcccccgcagccgaacggcatgaccaacaacctccagggcagcaacacctatgccctggagaacactatgatcttcaacagccagccggcgaacccgggcaccaccgccacgtacctcgagggcaacatgctcatcaccagcgagagcgagacgcagccggtgaaccgcgtggcgtacaacgtcggcgggcagatggccaccaacaaccagagctccaccactgcccccgcgaccggcacgtacaacctccaggaaatcgtgcccggcagcgtgtggatggagagggacgtgtacctccaaggacccatctgggccaagatcccagagacgggggcgcactttcacccctctccggccatgggcggattcggactcaaacacccaccgcccatgatgctcatcaagaacacgcctgtgcccggaaatatcaccagcttctcggacgtgcccgtcagcagcttcatcacccagtacagcaccgggcaggtcaccgtggagatggagtgggagctcaagaaggaaaactccaagaggtggaacccagagatccagtacacaaacaactacaacgacccccagtttgtggactttgccccggacagcaccggggaatacagaaccaccagacctatcggaacccgataccttacccgacccctttaattgcttgttaatcaataaaccgtttaattcgtttcagttgaactttggtctctgcgaagggcgaattcgtttaaacctgcaggactagaggtcctgtattagaggtcacgtgagtgttttgcgacattttgcgacaccatgtggtcacgctgggtatttaagcccgagtgagcacgcagggtctccattttgaagcgggaggtttgaacgcgcagccgccaagccgaattctgcagatccggttaattcgtttcagttgaactttggtctcctgtccttcttatcttatcggttaccatagaaactggttacttattaactgcttggtgcgcttcgcgataaaagacttacgtcatcgggttacccctagtgatggagcggccgctttcagttgaactttggtctctgcgtatttctttcttatctagtttccatgctctagaggtcctgtattagaggtcacgtgagtgttttgcgacattttgcgacaccatgtggtcacgctgggtatttaagcccgagtgagcacgcagggtctccattttgaagcgggaggtttgaacgcgcagccgccaagccgaattctgcagaatcccaatggcgcgccgagcttggctcgagcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccagggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 55 - DNA, 7068 bp, synthetic construct
  source 1..7068: mol_type = other DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgacgcgtattgggatcgcgcagccgccatgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggctcttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatgtcttttgttgatcaccctccagattggttggaagaagttggtgaaggtcttcgcgagtttttgggccttgaagcgggcccaccgaaaccaaaacccaatcagcagcatcaagatcaagcccgtggtcttgtgctgcctggttataactatctcggacccggaaacggtctcgatcgaggagagcctgtcaacagggcagacgaggtcgcgcgagagcacgacatctcgtacaacgagcagcttgaggcgggagacaacccctacctcaagtacaaccacgcggacgccgagtttcaggagaagctcgccgacgacacatccttcgggggaaacctcggaaaggcagtctttcaggccaagaaaagggttctcgaaccttttggcctggttgaagagggtgctaagacggcccctaccggaaagcggatagacgaccactttccaaaaagaaagaaggctcggaccgaagaggactccaagccttccacctcgtcagacgccgaagctggacccagcggatcccagcagctgcaaatcccagcccaaccagcctcaagtttgggagctgatacaatgtctgcgggaggtggcggcccattgggcgacaataaccaaggtgccgatggagtgggcaatgcctcgggagattggcattgcgattccacgtggatgggggacagagtcgtcaccaagtccacccgaacctgggtgctgcccagctacaacaaccaccagtaccgagagatcaaaagcggctccgtcgacggaagcaacgccaacgcctactttggatacagcaccccctgggggtactttgactttaaccgcttccacagccactggagcccccgagactggcaaagactcatcaacaactactggggcttcagaccccggtccctcagagtcaaaatcttcaacattcaagtcaaagaggtcacggtgcaggactccaccaccaccatcgccaacaacctcacctccaccgtccaagtgtttacggacgacgactaccagctgccctacgtcgtcggcaacgggaccgagggatgcctgccggccttccctccgcaggtctttacgctgccgcagtacggttacgcgacgctgaaccgcgacaacacagaaaatcccaccgagaggagcagcttcttctgcctagagtactttcccagcaagatgctgagaacgggcaacaactttgagtttacctacaactttgaggaggtgcccttccactccagcttcgctcccagtcagaacctgttcaagctggccaacccgctggtggaccagtacttgtaccgcttcgtgagcacaaataacactggcggagtccagttcaacaagaacctggccgggagatacgccaacacctacaaaaactggttcccggggcccatgggccgaacccagggctggaacctgggctccggggtcaaccgcgccagtgtcagcgccttcgccacgaccaataggatggagctcgagggcgcgagttaccaggtgcccccgcagccgaacggcatgaccaacaacctccagggcagcaacacctatgccctggagaacactatgatcttcaacagccagccggcgaacccgggcaccaccgccacgtacctcgagggcaacatgctcatcaccagcgagagcgagacgcagccggtgaaccgcgtggcgtacaacgtcggcgggcagatggccaccaacaaccagagctccaccactgcccccgcgaccggcacgtacaacctccaggaaatcgtgcccggcagcgtgtggatggagagggacgtgtacctccaaggacccatctgggccaagatcccagagacgggggcgcactttcacccctctccggccatgggcggattcggactcaaacacccaccgcccatgatgctcatcaagaacacgcctgtgcccggaaatatcaccagcttctcggacgtgcccgtcagcagcttcatcacccagtacagcaccgggcaggtcaccgtggagatggagtgggagctcaagaaggaaaactccaagaggtggaacccagagatccagtacacaaacaactacaacgacccccagtttgtggactttgccccggacagcaccggggaatacagaaccaccagacctatcggaacccgataccttacccgacccctttaattgcttgttaatcaataaaccgtttaattcgtttcagttgaactttggtctctgccctaggggaggtcctgtattagaggtcacgtgagtgttttgcgacattttgcgacaccatgtggtcacgctgggtatttaagcccgagtgagcacgcagggtctccattttgaagcgggaggtttgaacgcgcagccgccaagccgtttcagttgaactttggtctctgcgtatttctttcttatctagtttccatggaggtcctgtattagaggtcacgtgagtgttttgcgacattttgcgacaccatgtggtcacgctgggtatttaagcccgagtgagcacgcagggtctccattttgaagcgggaggtttgaaatcccaatggcgcgccgagcttggctcgagcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccagggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 56 - DNA, 7099 bp, synthetic construct
  source 1..7099: mol_type = other DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgacgcgtattgggatcgcgcagccgccatgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggctcttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatgtcttttgttgatcaccctccagattggttggaagaagttggtgaaggtcttcgcgagtttttgggccttgaagcgggcccaccgaaaccaaaacccaatcagcagcatcaagatcaagcccgtggtcttgtgctgcctggttataactatctcggacccggaaacggtctcgatcgaggagagcctgtcaacagggcagacgaggtcgcgcgagagcacgacatctcgtacaacgagcagcttgaggcgggagacaacccctacctcaagtacaaccacgcggacgccgagtttcaggagaagctcgccgacgacacatccttcgggggaaacctcggaaaggcagtctttcaggccaagaaaagggttctcgaaccttttggcctggttgaagagggtgctaagacggcccctaccggaaagcggatagacgaccactttccaaaaagaaagaaggctcggaccgaagaggactccaagccttccacctcgtcagacgccgaagctggacccagcggatcccagcagctgcaaatcccagcccaaccagcctcaagtttgggagctgatacaatgtctgcgggaggtggcggcccattgggcgacaataaccaaggtgccgatggagtgggcaatgcctcgggagattggcattgcgattccacgtggatgggggacagagtcgtcaccaagtccacccgaacctgggtgctgcccagctacaacaaccaccagtaccgagagatcaaaagcggctccgtcgacggaagcaacgccaacgcctactttggatacagcaccccctgggggtactttgactttaaccgcttccacagccactggagcccccgagactggcaaagactcatcaacaactactggggcttcagaccccggtccctcagagtcaaaatcttcaacattcaagtcaaagaggtcacggtgcaggactccaccaccaccatcgccaacaacctcacctccaccgtccaagtgtttacggacgacgactaccagctgccctacgtcgtcggcaacgggaccgagggatgcctgccggccttccctccgcaggtctttacgctgccgcagtacggttacgcgacgctgaaccgcgacaacacagaaaatcccaccgagaggagcagcttcttctgcctagagtactttcccagcaagatgctgagaacgggcaacaactttgagtttacctacaactttgaggaggtgcccttccactccagcttcgctcccagtcagaacctgttcaagctggccaacccgctggtggaccagtacttgtaccgcttcgtgagcacaaataacactggcggagtccagttcaacaagaacctggccgggagatacgccaacacctacaaaaactggttcccggggcccatgggccgaacccagggctggaacctgggctccggggtcaaccgcgccagtgtcagcgccttcgccacgaccaataggatggagctcgagggcgcgagttaccaggtgcccccgcagccgaacggcatgaccaacaacctccagggcagcaacacctatgccctggagaacactatgatcttcaacagccagccggcgaacccgggcaccaccgccacgtacctcgagggcaacatgctcatcaccagcgagagcgagacgcagccggtgaaccgcgtggcgtacaacgtcggcgggcagatggccaccaacaaccagagctccaccactgcccccgcgaccggcacgtacaacctccaggaaatcgtgcccggcagcgtgtggatggagagggacgtgtacctccaaggacccatctgggccaagatcccagagacgggggcgcactttcacccctctccggccatgggcggattcggactcaaacacccaccgcccatgatgctcatcaagaacacgcctgtgcccggaaatatcaccagcttctcggacgtgcccgtcagcagcttcatcacccagtacagcaccgggcaggtcaccgtggagatggagtgggagctcaagaaggaaaactccaagaggtggaacccagagatccagtacacaaacaactacaacgacccccagtttgtggactttgccccggacagcaccggggaatacagaaccaccagacctatcggaacccgataccttacccgacccctttaattgcttgttaatcaataaaccgtttaattcgtttcagttgaactttggtctctgccctaggggaggtcctgtattagaggtcacgtgagtgttttgcgacattttgcgacaccatgtggtcacgctgggtatttaagcccgagtgagcacgcagggtctccattttgaagcgggaggtttgaagtcttacccccaataaggggatttgctctatttaattaggaataaggtcgattactgatagaacaaatccaggctactgtgtttagtaatcagatttgttgaggtcctgtattagaggtcacgtgagtgttttgcgacattttgcgacaccatgtggtcacgctgggtatttaagcccgagtgagcacgcagggtctccattttgaagcgggaggtttgaaatcccaatggcgcgccgagcttggctcgagcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccagggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 57 - DNA, 7199 bp, synthetic construct
  source 1..7199: mol_type = other DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgacgcgtattgggatcgcgcagccgccatgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggctcttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatgtcttttgttgatcaccctccagattggttggaagaagttggtgaaggtcttcgcgagtttttgggccttgaagcgggcccaccgaaaccaaaacccaatcagcagcatcaagatcaagcccgtggtcttgtgctgcctggttataactatctcggacccggaaacggtctcgatcgaggagagcctgtcaacagggcagacgaggtcgcgcgagagcacgacatctcgtacaacgagcagcttgaggcgggagacaacccctacctcaagtacaaccacgcggacgccgagtttcaggagaagctcgccgacgacacatccttcgggggaaacctcggaaaggcagtctttcaggccaagaaaagggttctcgaaccttttggcctggttgaagagggtgctaagacggcccctaccggaaagcggatagacgaccactttccaaaaagaaagaaggctcggaccgaagaggactccaagccttccacctcgtcagacgccgaagctggacccagcggatcccagcagctgcaaatcccagcccaaccagcctcaagtttgggagctgatacaatgtctgcgggaggtggcggcccattgggcgacaataaccaaggtgccgatggagtgggcaatgcctcgggagattggcattgcgattccacgtggatgggggacagagtcgtcaccaagtccacccgaacctgggtgctgcccagctacaacaaccaccagtaccgagagatcaaaagcggctccgtcgacggaagcaacgccaacgcctactttggatacagcaccccctgggggtactttgactttaaccgcttccacagccactggagcccccgagactggcaaagactcatcaacaactactggggcttcagaccccggtccctcagagtcaaaatcttcaacattcaagtcaaagaggtcacggtgcaggactccaccaccaccatcgccaacaacctcacctccaccgtccaagtgtttacggacgacgactaccagctgccctacgtcgtcggcaacgggaccgagggatgcctgccggccttccctccgcaggtctttacgctgccgcagtacggttacgcgacgctgaaccgcgacaacacagaaaatcccaccgagaggagcagcttcttctgcctagagtactttcccagcaagatgctgagaacgggcaacaactttgagtttacctacaactttgaggaggtgcccttccactccagcttcgctcccagtcagaacctgttcaagctggccaacccgctggtggaccagtacttgtaccgcttcgtgagcacaaataacactggcggagtccagttcaacaagaacctggccgggagatacgccaacacctacaaaaactggttcccggggcccatgggccgaacccagggctggaacctgggctccggggtcaaccgcgccagtgtcagcgccttcgccacgaccaataggatggagctcgagggcgcgagttaccaggtgcccccgcagccgaacggcatgaccaacaacctccagggcagcaacacctatgccctggagaacactatgatcttcaacagccagccggcgaacccgggcaccaccgccacgtacctcgagggcaacatgctcatcaccagcgagagcgagacgcagccggtgaaccgcgtggcgtacaacgtcggcgggcagatggccaccaacaaccagagctccaccactgcccccgcgaccggcacgtacaacctccaggaaatcgtgcccggcagcgtgtggatggagagggacgtgtacctccaaggacccatctgggccaagatcccagagacgggggcgcactttcacccctctccggccatgggcggattcggactcaaacacccaccgcccatgatgctcatcaagaacacgcctgtgcccggaaatatcaccagcttctcggacgtgcccgtcagcagcttcatcacccagtacagcaccgggcaggtcaccgtggagatggagtgggagctcaagaaggaaaactccaagaggtggaacccagagatccagtacacaaacaactacaacgacccccagtttgtggactttgccccggacagcaccggggaatacagaaccaccagacctatcggaacccgataccttacccgacccctttaattgcttgttaatcaataaaccgtttaattcgtttcagttgaactttggtctctgccctaggggaggtcctgtattagaggtcacgtgagtgttttgcgacattttgcgacaccatgtggtcacgctgggtatttaagcccgagtgagcacgcagggtctccattttgaagcgggaggtttgaagtcttacccccaataaggggatttgctctatttaattaggaataaggtcgattactgatagaacaaatccaggctactgtgtttagtaatcagatttgttcgtgaccgatatgcacgggcaaaacggcaggaggttgttagcgcgacctcctgccacccgctttcacgaaggtcatgtgtaaaaggccgcagcgtaactagaggtcctgtattagaggtcacgtgagtgttttgcgacattttgcgacaccatgtggtcacgctgggtatttaagcccgagtgagcacgcagggtctccattttgaagcgggaggtttgaaatcccaatggcgcgccgagcttggctcgagcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccagggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 58 - DNA, 7299 bp, synthetic construct
  source 1..7299: mol_type = other DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgacgcgtattgggatcgcgcagccgccatgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggctcttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatgtcttttgttgatcaccctccagattggttggaagaagttggtgaaggtcttcgcgagtttttgggccttgaagcgggcccaccgaaaccaaaacccaatcagcagcatcaagatcaagcccgtggtcttgtgctgcctggttataactatctcggacccggaaacggtctcgatcgaggagagcctgtcaacagggcagacgaggtcgcgcgagagcacgacatctcgtacaacgagcagcttgaggcgggagacaacccctacctcaagtacaaccacgcggacgccgagtttcaggagaagctcgccgacgacacatccttcgggggaaacctcggaaaggcagtctttcaggccaagaaaagggttctcgaaccttttggcctggttgaagagggtgctaagacggcccctaccggaaagcggatagacgaccactttccaaaaagaaagaaggctcggaccgaagaggactccaagccttccacctcgtcagacgccgaagctggacccagcggatcccagcagctgcaaatcccagcccaaccagcctcaagtttgggagctgatacaatgtctgcgggaggtggcggcccattgggcgacaataaccaaggtgccgatggagtgggcaatgcctcgggagattggcattgcgattccacgtggatgggggacagagtcgtcaccaagtccacccgaacctgggtgctgcccagctacaacaaccaccagtaccgagagatcaaaagcggctccgtcgacggaagcaacgccaacgcctactttggatacagcaccccctgggggtactttgactttaaccgcttccacagccactggagcccccgagactggcaaagactcatcaacaactactggggcttcagaccccggtccctcagagtcaaaatcttcaacattcaagtcaaagaggtcacggtgcaggactccaccaccaccatcgccaacaacctcacctccaccgtccaagtgtttacggacgacgactaccagctgccctacgtcgtcggcaacgggaccgagggatgcctgccggccttccctccgcaggtctttacgctgccgcagtacggttacgcgacgctgaaccgcgacaacacagaaaatcccaccgagaggagcagcttcttctgcctagagtactttcccagcaagatgctgagaacgggcaacaactttgagtttacctacaactttgaggaggtgcccttccactccagcttcgctcccagtcagaacctgttcaagctggccaacccgctggtggaccagtacttgtaccgcttcgtgagcacaaataacactggcggagtccagttcaacaagaacctggccgggagatacgccaacacctacaaaaactggttcccggggcccatgggccgaacccagggctggaacctgggctccggggtcaaccgcgccagtgtcagcgccttcgccacgaccaataggatggagctcgagggcgcgagttaccaggtgcccccgcagccgaacggcatgaccaacaacctccagggcagcaacacctatgccctggagaacactatgatcttcaacagccagccggcgaacccgggcaccaccgccacgtacctcgagggcaacatgctcatcaccagcgagagcgagacgcagccggtgaaccgcgtggcgtacaacgtcggcgggcagatggccaccaacaaccagagctccaccactgcccccgcgaccggcacgtacaacctccaggaaatcgtgcccggcagcgtgtggatggagagggacgtgtacctccaaggacccatctgggccaagatcccagagacgggggcgcactttcacccctctccggccatgggcggattcggactcaaacacccaccgcccatgatgctcatcaagaacacgcctgtgcccggaaatatcaccagcttctcggacgtgcccgtcagcagcttcatcacccagtacagcaccgggcaggtcaccgtggagatggagtgggagctcaagaaggaaaactccaagaggtggaacccagagatccagtacacaaacaactacaacgacccccagtttgtggactttgccccggacagcaccggggaatacagaaccaccagacctatcggaacccgataccttacccgacccctttaattgcttgttaatcaataaaccgtttaattcgtttcagttgaactttggtctctgccctaggggaggtcctgtattagaggtcacgtgagtgttttgcgacattttgcgacaccatgtggtcacgctgggtatttaagcccgagtgagcacgcagggtctccattttgaagcgggaggtttgaagtcttacccccaataaggggatttgctctatttaattaggaataaggtcgattactgatagaacaaatccaggctactgtgtttagtaatcagatttgttcgtgaccgatatgcacgggcaaaacggcaggaggttgttagcgcgacctcctgccacccgctttcacgaaggtcatgtgtaaaaggccgcagcgtaactattactaatgaattcaggacagacagtggctacggctcagtttgggttgtgctgttgctgggcggcgatgacgcctgtacgcatttggtgatccggttctggaggtcctgtattagaggtcacgtgagtgttttgcgacattttgcgacaccatgtggtcacgctgggtatttaagcccgagtgagcacgcagggtctccattttgaagcgggaggtttgaaatcccaatggcgcgccgagcttggctcgagcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccagggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 59 - DNA, 7099 bp, synthetic construct
  source 1..7099: mol_type = other DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgacgcgtattgggatcgcgcagccgccatgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggctcttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatgtcttttgttgatcaccctccagattggttggaagaagttggtgaaggtcttcgcgagtttttgggccttgaagcgggcccaccgaaaccaaaacccaatcagcagcatcaagatcaagcccgtggtcttgtgctgcctggttataactatctcggacccggaaacggtctcgatcgaggagagcctgtcaacagggcagacgaggtcgcgcgagagcacgacatctcgtacaacgagcagcttgaggcgggagacaacccctacctcaagtacaaccacgcggacgccgagtttcaggagaagctcgccgacgacacatccttcgggggaaacctcggaaaggcagtctttcaggccaagaaaagggttctcgaaccttttggcctggttgaagagggtgctaagacggcccctaccggaaagcggatagacgaccactttccaaaaagaaagaaggctcggaccgaagaggactccaagccttccacctcgtcagacgccgaagctggacccagcggatcccagcagctgcaaatcccagcccaaccagcctcaagtttgggagctgatacaatgtctgcgggaggtggcggcccattgggcgacaataaccaaggtgccgatggagtgggcaatgcctcgggagattggcattgcgattccacgtggatgggggacagagtcgtcaccaagtccacccgaacctgggtgctgcccagctacaacaaccaccagtaccgagagatcaaaagcggctccgtcgacggaagcaacgccaacgcctactttggatacagcaccccctgggggtactttgactttaaccgcttccacagccactggagcccccgagactggcaaagactcatcaacaactactggggcttcagaccccggtccctcagagtcaaaatcttcaacattcaagtcaaagaggtcacggtgcaggactccaccaccaccatcgccaacaacctcacctccaccgtccaagtgtttacggacgacgactaccagctgccctacgtcgtcggcaacgggaccgagggatgcctgccggccttccctccgcaggtctttacgctgccgcagtacggttacgcgacgctgaaccgcgacaacacagaaaatcccaccgagaggagcagcttcttctgcctagagtactttcccagcaagatgctgagaacgggcaacaactttgagtttacctacaactttgaggaggtgcccttccactccagcttcgctcccagtcagaacctgttcaagctggccaacccgctggtggaccagtacttgtaccgcttcgtgagcacaaataacactggcggagtccagttcaacaagaacctggccgggagatacgccaacacctacaaaaactggttcccggggcccatgggccgaacccagggctggaacctgggctccggggtcaaccgcgccagtgtcagcgccttcgccacgaccaataggatggagctcgagggcgcgagttaccaggtgcccccgcagccgaacggcatgaccaacaacctccagggcagcaacacctatgccctggagaacactatgatcttcaacagccagccggcgaacccgggcaccaccgccacgtacctcgagggcaacatgctcatcaccagcgagagcgagacgcagccggtgaaccgcgtggcgtacaacgtcggcgggcagatggccaccaacaaccagagctccaccactgcccccgcgaccggcacgtacaacctccaggaaatcgtgcccggcagcgtgtggatggagagggacgtgtacctccaaggacccatctgggccaagatcccagagacgggggcgcactttcacccctctccggccatgggcggattcggactcaaacacccaccgcccatgatgctcatcaagaacacgcctgtgcccggaaatatcaccagcttctcggacgtgcccgtcagcagcttcatcacccagtacagcaccgggcaggtcaccgtggagatggagtgggagctcaagaaggaaaactccaagaggtggaacccagagatccagtacacaaacaactacaacgacccccagtttgtggactttgccccggacagcaccggggaatacagaaccaccagacctatcggaacccgataccttacccgacccctttaattgcttgttaatcaataaaccgtttaattcgtttcagttgaactttggtctctgccctaggggaggtcctgtattagaggtcacgtgagtgttttgcgacattttgcgacaccatgtggtcacgctgggtatttaagcccgagtgagcacgcagggtctccattttgaagcgggaggtttgaacatacagacgaaactagttggagattatcgagcattctatcacgtcgacgaccactagtgagttactagagccgaggggtaacgttgatgctcctaaaaagaggtcctgtattagaggtcacgtgagtgttttgcgacattttgcgacaccatgtggtcacgctgggtatttaagcccgagtgagcacgcagggtctccattttgaagcgggaggtttgaaatcccaatggcgcgccgagcttggctcgagcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccagggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 60 - DNA, 7199 bp, synthetic construct
  source 1..7199: mol_type = other DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgacgcgtattgggatcgcgcagccgccatgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggctcttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatgtcttttgttgatcaccctccagattggttggaagaagttggtgaaggtcttcgcgagtttttgggccttgaagcgggcccaccgaaaccaaaacccaatcagcagcatcaagatcaagcccgtggtcttgtgctgcctggttataactatctcggacccggaaacggtctcgatcgaggagagcctgtcaacagggcagacgaggtcgcgcgagagcacgacatctcgtacaacgagcagcttgaggcgggagacaacccctacctcaagtacaaccacgcggacgccgagtttcaggagaagctcgccgacgacacatccttcgggggaaacctcggaaaggcagtctttcaggccaagaaaagggttctcgaaccttttggcctggttgaagagggtgctaagacggcccctaccggaaagcggatagacgaccactttccaaaaagaaagaaggctcggaccgaagaggactccaagccttccacctcgtcagacgccgaagctggacccagcggatcccagcagctgcaaatcccagcccaaccagcctcaagtttgggagctgatacaatgtctgcgggaggtggcggcccattgggcgacaataaccaaggtgccgatggagtgggcaatgcctcgggagattggcattgcgattccacgtggatgggggacagagtcgtcaccaagtccacccgaacctgggtgctgcccagctacaacaaccaccagtaccgagagatcaaaagcggctccgtcgacggaagcaacgccaacgcctactttggatacagcaccccctgggggtactttgactttaaccgcttccacagccactggagcccccgagactggcaaagactcatcaacaactactggggcttcagaccccggtccctcagagtcaaaatcttcaacattcaagtcaaagaggtcacggtgcaggactccaccaccaccatcgccaacaacctcacctccaccgtccaagtgtttacggacgacgactaccagctgccctacgtcgtcggcaacgggaccgagggatgcctgccggccttccctccgcaggtctttacgctgccgcagtacggttacgcgacgctgaaccgcgacaacacagaaaatcccaccgagaggagcagcttcttctgcctagagtactttcccagcaagatgctgagaacgggcaacaactttgagtttacctacaactttgaggaggtgcccttccactccagcttcgctcccagtcagaacctgttcaagctggccaacccgctggtggaccagtacttgtaccgcttcgtgagcacaaataacactggcggagtccagttcaacaagaacctggccgggagatacgccaacacctacaaaaactggttcccggggcccatgggccgaacccagggctggaacctgggctccggggtcaaccgcgccagtgtcagcgccttcgccacgaccaataggatggagctcgagggcgcgagttaccaggtgcccccgcagccgaacggcatgaccaacaacctccagggcagcaacacctatgccctggagaacactatgatcttcaacagccagccggcgaacccgggcaccaccgccacgtacctcgagggcaacatgctcatcaccagcgagagcgagacgcagccggtgaaccgcgtggcgtacaacgtcggcgggcagatggccaccaacaaccagagctccaccactgcccccgcgaccggcacgtacaacctccaggaaatcgtgcccggcagcgtgtggatggagagggacgtgtacctccaaggacccatctgggccaagatcccagagacgggggcgcactttcacccctctccggccatgggcggattcggactcaaacacccaccgcccatgatgctcatcaagaacacgcctgtgcccggaaatatcaccagcttctcggacgtgcccgtcagcagcttcatcacccagtacagcaccgggcaggtcaccgtggagatggagtgggagctcaagaaggaaaactccaagaggtggaacccagagatccagtacacaaacaactacaacgacccccagtttgtggactttgccccggacagcaccggggaatacagaaccaccagacctatcggaacccgataccttacccgacccctttaattgcttgttaatcaataaaccgtttaattcgtttcagttgaactttggtctctgccctaggggaggtcctgtattagaggtcacgtgagtgttttgcgacattttgcgacaccatgtggtcacgctgggtatttaagcccgagtgagcacgcagggtctccattttgaagcgggaggtttgaacatacagacgaaactagttggagattatcgagcattctatcacgtcgacgaccactagtgagttactagagccgaggggtaacgttgatgctcctaaaaatctctcggtcgatgcaagcgattacactcctgtcacatcataatcgtttgctattcagggcttgatcaacactggattgcttttcacttaaagtattatggaggtcctgtattagaggtcacgtgagtgttttgcgacattttgcgacaccatgtggtcacgctgggtatttaagcccgagtgagcacgcagggtctccattttgaagcgggaggtttgaaatcccaatggcgcgccgagcttggctcgagcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccagggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 61 - DNA, 7299 bp, synthetic construct
  source 1..7299: mol_type = other DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgacgcgtattgggatcgcgcagccgccatgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggctcttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatgtcttttgttgatcaccctccagattggttggaagaagttggtgaaggtcttcgcgagtttttgggccttgaagcgggcccaccgaaaccaaaacccaatcagcagcatcaagatcaagcccgtggtcttgtgctgcctggttataactatctcggacccggaaacggtctcgatcgaggagagcctgtcaacagggcagacgaggtcgcgcgagagcacgacatctcgtacaacgagcagcttgaggcgggagacaacccctacctcaagtacaaccacgcggacgccgagtttcaggagaagctcgccgacgacacatccttcgggggaaacctcggaaaggcagtctttcaggccaagaaaagggttctcgaaccttttggcctggttgaagagggtgctaagacggcccctaccggaaagcggatagacgaccactttccaaaaagaaagaaggctcggaccgaagaggactccaagccttccacctcgtcagacgccgaagctggacccagcggatcccagcagctgcaaatcccagcccaaccagcctcaagtttgggagctgatacaatgtctgcgggaggtggcggcccattgggcgacaataaccaaggtgccgatggagtgggcaatgcctcgggagattggcattgcgattccacgtggatgggggacagagtcgtcaccaagtccacccgaacctgggtgctgcccagctacaacaaccaccagtaccgagagatcaaaagcggctccgtcgacggaagcaacgccaacgcctactttggatacagcaccccctgggggtactttgactttaaccgcttccacagccactggagcccccgagactggcaaagactcatcaacaactactggggcttcagaccccggtccctcagagtcaaaatcttcaacattcaagtcaaagaggtcacggtgcaggactccaccaccaccatcgccaacaacctcacctccaccgtccaagtgtttacggacgacgactaccagctgccctacgtcgtcggcaacgggaccgagggatgcctgccggccttccctccgcaggtctttacgctgccgcagtacggttacgcgacgctgaaccgcgacaacacagaaaatcccaccgagaggagcagcttcttctgcctagagtactttcccagcaagatgctgagaacgggcaacaactttgagtttacctacaactttgaggaggtgcccttccactccagcttcgctcccagtcagaacctgttcaagctggccaacccgctggtggaccagtacttgtaccgcttcgtgagcacaaataacactggcggagtccagttcaacaagaacctggccgggagatacgccaacacctacaaaaactggttcccggggcccatgggccgaacccagggctggaacctgggctccggggtcaaccgcgccagtgtcagcgccttcgccacgaccaataggatggagctcgagggcgcgagttaccaggtgcccccgcagccgaacggcatgaccaacaacctccagggcagcaacacctatgccctggagaacactatgatcttcaacagccagccggcgaacccgggcaccaccgccacgtacctcgagggcaacatgctcatcaccagcgagagcgagacgcagccggtgaaccgcgtggcgtacaacgtcggcgggcagatggccaccaacaaccagagctccaccactgcccccgcgaccggcacgtacaacctccaggaaatcgtgcccggcagcgtgtggatggagagggacgtgtacctccaaggacccatctgggccaagatcccagagacgggggcgcactttcacccctctccggccatgggcggattcggactcaaacacccaccgcccatgatgctcatcaagaacacgcctgtgcccggaaatatcaccagcttctcggacgtgcccgtcagcagcttcatcacccagtacagcaccgggcaggtcaccgtggagatggagtgggagctcaagaaggaaaactccaagaggtggaacccagagatccagtacacaaacaactacaacgacccccagtttgtggactttgccccggacagcaccggggaatacagaaccaccagacctatcggaacccgataccttacccgacccctttaattgcttgttaatcaataaaccgtttaattcgtttcagttgaactttggtctctgccctaggggaggtcctgtattagaggtcacgtgagtgttttgcgacattttgcgacaccatgtggtcacgctgggtatttaagcccgagtgagcacgcagggtctccattttgaagcgggaggtttgaacatacagacgaaactagttggagattatcgagcattctatcacgtcgacgaccactagtgagttactagagccgaggggtaacgttgatgctcctaaaaatctctcggtcgatgcaagcgattacactcctgtcacatcataatcgtttgctattcagggcttgatcaacactggattgcttttcacttaaagtattatgcacgacagggtacgtgtaccatataaacctgttataacttacctcagactagttggaaatgtggctagatcttagcttacgttactaaagggtccacgttgaggtcctgtattagaggtcacgtgagtgttttgcgacattttgcgacaccatgtggtcacgctgggtatttaagcccgagtgagcacgcagggtctccattttgaagcgggaggtttgaaatcccaatggcgcgccgagcttggctcgagcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccagggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 62 - DNA, 7258 bp, synthetic construct
  source 1..7258: mol_type = other DNA
atgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggctcttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatgtcttttgttgatcaccctccagattggttggaagaagttggtgaaggtcttcgcgagtttttgggccttgaagcgggcccaccgaaaccaaaacccaatcagcagcatcaagatcaagcccgtggtcttgtgctgcctggttataactatctcggacccggaaacggtctcgatcgaggagagcctgtcaacagggcagacgaggtcgcgcgagagcacgacatctcgtacaacgagcagcttgaggcgggagacaacccctacctcaagtacaaccacgcggacgccgagtttcaggagaagctcgccgacgacacatccttcgggggaaacctcggaaaggcagtctttcaggccaagaaaagggttctcgaaccttttggcctggttgaagagggtgctaagacggcccctaccggaaagcggatagacgaccactttccaaaaagaaagaaggctcggaccgaagaggactccaagccttccacctcgtcagacgccgaagctggacccagcggatcccagcagctgcaaatcccagcccaaccagcctcaagtttgggagctgatacaatgtctgcgggaggtggcggcccattgggcgacaataaccaaggtgccgatggagtgggcaatgcctcgggagattggcattgcgattccacgtggatgggggacagagtcgtcaccaagtccacccgaacctgggtgctgcccagctacaacaaccaccagtaccgagagatcaaaagcggctccgtcgacggaagcaacgccaacgcctactttggatacagcaccccctgggggtactttgactttaaccgcttccacagccactggagcccccgagactggcaaagactcatcaacaactactggggcttcagaccccggtccctcagagtcaaaatcttcaacattcaagtcaaagaggtcacggtgcaggactccaccaccaccatcgccaacaacctcacctccaccgtccaagtgtttacggacgacgactaccagctgccctacgtcgtcggcaacgggaccgagggatgcctgccggccttccctccgcaggtctttacgctgccgcagtacggttacgcgacgctgaaccgcgacaacacagaaaatcccaccgagaggagcagcttcttctgcctagagtactttcccagcaagatgctgagaacgggcaacaactttgagtttacctacaactttgaggaggtgcccttccactccagcttcgctcccagtcagaacctgttcaagctggccaacccgctggtggaccagtacttgtaccgcttcgtgagcacaaataacactggcggagtccagttcaacaagaacctggccgggagatacgccaacacctacaaaaactggttcccggggcccatgggccgaacccagggctggaacctgggctccggggtcaaccgcgccagtgtcagcgccttcgccacgaccaataggatggagctcgagggcgcgagttaccaggtgcccccgcagccgaacggcatgaccaacaacctccagggcagcaacacctatgccctggagaacactatgatcttcaacagccagccggcgaacccgggcaccaccgccacgtacctcgagggcaacatgctcatcaccagcgagagcgagacgcagccggtgaaccgcgtggcgtacaacgtcggcgggcagatggccaccaacaaccagagctccaccactgcccccgcgaccggcacgtacaacctccaggaaatcgtgcccggcagcgtgtggatggagagggacgtgtacctccaaggacccatctgggccaagatcccagagacgggggcgcactttcacccctctccggccatgggcggattcggactcaaacacccaccgcccatgatgctcatcaagaacacgcctgtgcccggaaatatcaccagcttctcggacgtgcccgtcagcagcttcatcacccagtacagcaccgggcaggtcaccgtggagatggagtgggagctcaagaaggaaaactccaagaggtggaacccagagatccagtacacaaacaactacaacgacccccagtttgtggactttgccccggacagcaccggggaatacagaaccaccagacctatcggaacccgataccttacccgacccctttaattgcttgttaatcaataaaccgtttaattcgtttcagttgaactttggtctctgccctaggggaggtcctgtattagaggtcacgtgagtgttttgcgacattttgcgacaccatgtggtcacgctgggtatttaagcccgagtgagcacgcagggtctccattttgaagcgggaggtttgaacgcgcagccgccaagccgtttcagttgaactttggtctctgcgtatttctttcttatctagtttccatggaggtcctgtattagaggtcacgtgagtgttttgcgacattttgcgacaccatgtggtcacgctgggtatttaagcccgagtgagcacgcagggtctccattttgaagcgggaggtttgaacgcgcagccgccaagccgtttcagttgaactttggtctctgcgtatttctttcttatctagtttccatggaggtcctgtattagaggtcacgtgagtgttttgcgacattttgcgacaccatgtggtcacgctgggtatttaagcccgagtgagcacgcagggtctccattttgaagcgggaggtttgaaatcccaatggcgcgccgagcttggctcgagcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccagggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgacgcgtattgggatcgcgcagccgcc

SEQ ID NO: 63 - DNA, 6905 bp, synthetic construct
  source 1..6905: mol_type = other DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgacgcgtattgggatcgcgcagccgccatgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggctcttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgtaattgcttgttaatcaataaaccgtttaattcgtttcagttgaactttggtctctgcggaggtcctgtattagaggtcacgtgagtgttttgcgacattttgcgacaccatgtggtcacgctgggtatttaagcccgagtgagcacgcagggtctccattttgaagcgggaggtttgaaatcccaatggcgcgccgagcttggctcgagcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccagggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 64 - DNA, 7440 bp, synthetic construct
  source 1..7440: mol_type = other DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgacgcgtattgggatgaggtcctgtattagaggtcacgtgagtgttttgcgacattttgcgacaccatgtggtcacgctgggtatttaagcccgagtgagcacgcagggtctccattttgaagcgggaggtttgaacgcgcagccgccatgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggctcttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgtaattgcttgttaatcaataaaccgtttaattcgtttcagttgaactttggtctctgcgaagggcgaattcgtttaaacctgcaggactagaggtcctgtattagaggtcacgtgagtgttttgcgacattttgcgacaccatgtggtcacgctgggtatttaagcccgagtgagcacgcagggtctccattttgaagcgggaggtttgaacgcgcagccgccaagccgaattctgcagatccggttaattcgtttcagttgaactttggtctcctgtccttcttatcttatcggttaccatagaaactggttacttattaactgcttggtgcgcttcgcgataaaagacttacgtcatcgggttacccctagtgatggagcggccgctttcagttgaactttggtctctgcgtatttctttcttatctagtttccatgctctagaggtcctgtattagaggtcacgtgagtgttttgcgacattttgcgacaccatgtggtcacgctgggtatttaagcccgagtgagcacgcagggtctccattttgaagcgggaggtttgaacgcgcagccgccaagccgaattctgcagaatcccaatggcgcgccgagcttggctcgagcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccagggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 65 - DNA, 7440 bp, synthetic construct
  source 1..7440: mol_type = other DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgacgcgtattgggatgaggtcctgtattagaggtcacgtgagtgttttgcgacattttgcgacaccatgtggtcacgctgggtatttaagcccgagtgagcacgcagggtctccattttgaagcgggaggtttgaacgcgcagccgccacgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggctcttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgtaattgcttgttaatcaataaaccgtttaattcgtttcagttgaactttggtctctgcgaagggcgaattcgtttaaacctgcaggactagaggtcctgtattagaggtcacgtgagtgttttgcgacattttgcgacaccatgtggtcacgctgggtatttaagcccgagtgagcacgcagggtctccattttgaagcgggaggtttgaacgcgcagccgccaagccgaattctgcagatccggttaattcgtttcagttgaactttggtctcctgtccttcttatcttatcggttaccatagaaactggttacttattaactgcttggtgcgcttcgcgataaaagacttacgtcatcgggttacccctagtgatggagcggccgctttcagttgaactttggtctctgcgtatttctttcttatctagtttccatgctctagaggtcctgtattagaggtcacgtgagtgttttgcgacattttgcgacaccatgtggtcacgctgggtatttaagcccgagtgagcacgcagggtctccattttgaagcgggaggtttgaacgcgcagccgccaagccgaattctgcagaatcccaatggcgcgccgagcttggctcgagcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccagggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 66 - DNA, 7319 bp, synthetic construct
  source 1..7319: mol_type = other DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgacgcgtattgggatcgcgcagccgccatgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggctcttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgtaattgcttgttaatcaataaaccgtttaattcgtttcagttgaactttggtctctgcgaagggcgaattcgtttaaacctgcaggactagaggtcctgtattagaggtcacgtgagtgttttgcgacattttgcgacaccatgtggtcacgctgggtatttaagcccgagtgagcacgcagggtctccattttgaagcgggaggtttgaacgcgcagccgccaagccgaattctgcagatccggttaattcgtttcagttgaactttggtctcctgtccttcttatcttatcggttaccatagaaactggttacttattaactgcttggtgcgcttcgcgataaaagacttacgtcatcgggttacccctagtgatggagcggccgctttcagttgaactttggtctctgcgtatttctttcttatctagtttccatgctctagaggtcctgtattagaggtcacgtgagtgttttgcgacattttgcgacaccatgtggtcacgctgggtatttaagcccgagtgagcacgcagggtctccattttgaagcgggaggtttgaacgcgcagccgccaagccgaattctgcagaatcccaatggcgcgccgagcttggctcgagcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccagggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 67 - DNA, 7101 bp, synthetic construct
  source 1..7101: mol_type = other DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgacgcgtattgggatcgcgcagccgccatgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggctcttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgtaattgcttgttaatcaataaaccgtttaattcgtttcagttgaactttggtctctgccctaggggaggtcctgtattagaggtcacgtgagtgttttgcgacattttgcgacaccatgtggtcacgctgggtatttaagcccgagtgagcacgcagggtctccattttgaagcgggaggtttgaacgcgcagccgccaagccgtttcagttgaactttggtctctgcgtatttctttcttatctagtttccatggaggtcctgtattagaggtcacgtgagtgttttgcgacattttgcgacaccatgtggtcacgctgggtatttaagcccgagtgagcacgcagggtctccattttgaagcgggaggtttgaaatcccaatggcgcgccgagcttggctcgagcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccagggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 68 - DNA, 7132 bp, synthetic construct
  source 1..7132: mol_type = other DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgacgcgtattgggatcgcgcagccgccatgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggctcttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgtaattgcttgttaatcaataaaccgtttaattcgtttcagttgaactttggtctctgccctaggggaggtcctgtattagaggtcacgtgagtgttttgcgacattttgcgacaccatgtggtcacgctgggtatttaagcccgagtgagcacgcagggtctccattttgaagcgggaggtttgaagtcttacccccaataaggggatttgctctatttaattaggaataaggtcgattactgatagaacaaatccaggctactgtgtttagtaatcagatttgttgaggtcctgtattagaggtcacgtgagtgttttgcgacattttgcgacaccatgtggtcacgctgggtatttaagcccgagtgagcacgcagggtctccattttgaagcgggaggtttgaaatcccaatggcgcgccgagcttggctcgagcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccagggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 69 - DNA, 7232 bp, synthetic construct
  source 1..7232: mol_type = other DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgacgcgtattgggatcgcgcagccgccatgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggctcttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgtaattgcttgttaatcaataaaccgtttaattcgtttcagttgaactttggtctctgccctaggggaggtcctgtattagaggtcacgtgagtgttttgcgacattttgcgacaccatgtggtcacgctgggtatttaagcccgagtgagcacgcagggtctccattttgaagcgggaggtttgaagtcttacccccaataaggggatttgctctatttaattaggaataaggtcgattactgatagaacaaatccaggctactgtgtttagtaatcagatttgttcgtgaccgatatgcacgggcaaaacggcaggaggttgttagcgcgacctcctgccacccgctttcacgaaggtcatgtgtaaaaggccgcagcgtaactagaggtcctgtattagaggtcacgtgagtgttttgcgacattttgcgacaccatgtggtcacgctgggtatttaagcccgagtgagcacgcagggtctccattttgaagcgggaggtttgaaatcccaatggcgcgccgagcttggctcgagcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccagggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 70 - DNA, 7332 bp, synthetic construct
  source 1..7332: mol_type = other DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgacgcgtattgggatcgcgcagccgccatgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggctcttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgtaattgcttgttaatcaataaaccgtttaattcgtttcagttgaactttggtctctgccctaggggaggtcctgtattagaggtcacgtgagtgttttgcgacattttgcgacaccatgtggtcacgctgggtatttaagcccgagtgagcacgcagggtctccattttgaagcgggaggtttgaagtcttacccccaataaggggatttgctctatttaattaggaataaggtcgattactgatagaacaaatccaggctactgtgtttagtaatcagatttgttcgtgaccgatatgcacgggcaaaacggcaggaggttgttagcgcgacctcctgccacccgctttcacgaaggtcatgtgtaaaaggccgcagcgtaactattactaatgaattcaggacagacagtggctacggctcagtttgggttgtgctgttgctgggcggcgatgacgcctgtacgcatttggtgatccggttctggaggtcctgtattagaggtcacgtgagtgttttgcgacattttgcgacaccatgtggtcacgctgggtatttaagcccgagtgagcacgcagggtctccattttgaagcgggaggtttgaaatcccaatggcgcgccgagcttggctcgagcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccagggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 71 - DNA, 7132 bp, synthetic construct
  source 1..7132: mol_type = other DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgacgcgtattgggatcgcgcagccgccatgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggctcttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgtaattgcttgttaatcaataaaccgtttaattcgtttcagttgaactttggtctctgccctaggggaggtcctgtattagaggtcacgtgagtgttttgcgacattttgcgacaccatgtggtcacgctgggtatttaagcccgagtgagcacgcagggtctccattttgaagcgggaggtttgaacatacagacgaaactagttggagattatcgagcattctatcacgtcgacgaccactagtgagttactagagccgaggggtaacgttgatgctcctaaaaagaggtcctgtattagaggtcacgtgagtgttttgcgacattttgcgacaccatgtggtcacgctgggtatttaagcccgagtgagcacgcagggtctccattttgaagcgggaggtttgaaatcccaatggcgcgccgagcttggctcgagcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccagggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 72 - DNA, 7232 bp, synthetic construct
  source 1..7232: mol_type = other DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgacgcgtattgggatcgcgcagccgccatgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggctcttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgtaattgcttgttaatcaataaaccgtttaattcgtttcagttgaactttggtctctgccctaggggaggtcctgtattagaggtcacgtgagtgttttgcgacattttgcgacaccatgtggtcacgctgggtatttaagcccgagtgagcacgcagggtctccattttgaagcgggaggtttgaacatacagacgaaactagttggagattatcgagcattctatcacgtcgacgaccactagtgagttactagagccgaggggtaacgttgatgctcctaaaaatctctcggtcgatgcaagcgattacactcctgtcacatcataatcgtttgctattcagggcttgatcaacactggattgcttttcacttaaagtattatggaggtcctgtattagaggtcacgtgagtgttttgcgacattttgcgacaccatgtggtcacgctgggtatttaagcccgagtgagcacgcagggtctccattttgaagcgggaggtttgaaatcccaatggcgcgccgagcttggctcgagcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccagggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 73 - DNA, 7332 bp, synthetic construct
  source 1..7332: mol_type = other DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgacgcgtattgggatcgcgcagccgccatgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggctcttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgtaattgcttgttaatcaataaaccgtttaattcgtttcagttgaactttggtctctgccctaggggaggtcctgtattagaggtcacgtgagtgttttgcgacattttgcgacaccatgtggtcacgctgggtatttaagcccgagtgagcacgcagggtctccattttgaagcgggaggtttgaacatacagacgaaactagttggagattatcgagcattctatcacgtcgacgaccactagtgagttactagagccgaggggtaacgttgatgctcctaaaaatctctcggtcgatgcaagcgattacactcctgtcacatcataatcgtttgctattcagggcttgatcaacactggattgcttttcacttaaagtattatgcacgacagggtacgtgtaccatataaacctgttataacttacctcagactagttggaaatgtggctagatcttagcttacgttactaaagggtccacgttgaggtcctgtattagaggtcacgtgagtgttttgcgacattttgcgacaccatgtggtcacgctgggtatttaagcccgagtgagcacgcagggtctccattttgaagcgggaggtttgaaatcccaatggcgcgccgagcttggctcgagcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccagggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 74 - DNA, 7291 bp, synthetic construct
  source 1..7291: mol_type = other DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgacgcgtattgggatcgcgcagccgccatgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggctcttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgtaattgcttgttaatcaataaaccgtttaattcgtttcagttgaactttggtctctgccctaggggaggtcctgtattagaggtcacgtgagtgttttgcgacattttgcgacaccatgtggtcacgctgggtatttaagcccgagtgagcacgcagggtctccattttgaagcgggaggtttgaacgcgcagccgccaagccgtttcagttgaactttggtctctgcgtatttctttcttatctagtttccatggaggtcctgtattagaggtcacgtgagtgttttgcgacattttgcgacaccatgtggtcacgctgggtatttaagcccgagtgagcacgcagggtctccattttgaagcgggaggtttgaacgcgcagccgccaagccgtttcagttgaactttggtctctgcgtatttctttcttatctagtttccatggaggtcctgtattagaggtcacgtgagtgttttgcgacattttgcgacaccatgtggtcacgctgggtatttaagcccgagtgagcacgcagggtctccattttgaagcgggaggtttgaaatcccaatggcgcgccgagcttggctcgagcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccagggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

(19) United States
(12) Patent Application Publication
(10) Pub. No.: US 2026/0258449 A1
(43) Pub. Date: Sep. 3, 2026

(54) PACKAGING PLASMIDS FOR AAV PRODUCTION

(71) Applicant: Biogen MA Inc., Cambridge, MA (US)

(72) Inventors: Terrence Michael Dobrowsky, Waltham, MA (US); Venkata Soundarya Kandala, Cambridge, MA (US); Yizhou Zhou, Plainsboro, NJ (US)

(21) Appl. No.: 19/163,794

(22) PCT Filed: Mar. 8, 2024
(86) PCT No.: PCT/US2024/019262

§ 371 Date: Sep. 9, 2025

Related U.S. Application Data
(60) Provisional application No. 63/451,295, filed on Mar. 10, 2023.
(60) Provisional application No. 63/451,761, filed on Mar. 13, 2023.

(51) Int. Cl. C12N 15/864; C07K 14/075; C12N 7/02

(52) U.S. Cl. CPC C12N 15/8645; C07K 14/075; C12N 7/025; C12N 2710/10022; C12N 2710/10044; C12N 2750/14143; C12N 2750/14152; C12N 2800/10; C12N 2830/34

(57) ABSTRACT

The present disclosure provides improved packaging plasmids useful in the production of recombinant adeno-associated viruses.

### CROSS REFERENCE TO RELATED APPLICATIONS

This application claims priority to and the benefit of U.S. Provisional Patent Application No. 63/451,295 filed on Mar. 10, 2023, and U.S. Provisional Patent Application No. 63/451,761 filed on Mar. 13, 2023 the entire contents each of which are hereby incorporated by reference in their entirety.

### BACKGROUND

Gene therapy is a promising cure for many diseases such as genetic disorders. Recombinant adeno-associated viral vectors (rAAV) are being developed as a gene delivery vehicle in the gene therapy field. Production of rAAV vectors relies on introduction of plasmids (e.g., transfection) into a host cell. Improvements to the plasmids used in rAAV production can provide benefits for generating gene therapy vectors.

### SUMMARY

Commercial scale production of rAAV vectors is critical to clinical use of rAAV based gene therapy vectors. Modifications to the components of the rAAV production process that increase titer would be important contributions to the filed. The present disclosure recognizes that modification to promoters in plasmids used to generate rAAV gene therapy vectors can improve rAAV production.

In some embodiments, the present disclosure provides modifications and improvements to a packaging plasmid. In some embodiments, the present disclosure provides modifications and improvements to plasmids comprising replication (Rep) and capsid (Cap) sequences. In some embodiments, the present disclosure provides modifications and improvements to plasmids comprising both packaging plasmid sequences and adenoviral helper plasmid sequences. In some embodiments, the present disclosure provides modifications and improvements to a single plasmid comprising replication, capsid, and adenoviral helper sequences.

In some embodiments, the present disclosure recognizes modifications to promoter sequences in the packaging plasmid can improve rAAV production. In some embodiments, the present disclosure recognizes modifications to promoter sequence(s) of the packaging plasmid can increase rAAV titer. In some embodiments, the present disclosure recognizes modifications to the position, location, or number of p5 promoter(s) in a packaging plasmid can increase rAAV titer.

In some embodiments, the present disclosure provides a packaging plasmid comprising a coding region and at least one p5 promoter sequence downstream of a coding region. In some embodiments, a packaging plasmid comprises one p5 promoter sequence downstream of the coding region. In some embodiments, a packaging plasmid comprises two p5 promoter sequences, a first p5 promoter sequence and a second p5 promoter sequence, downstream of the coding region. In some embodiments, a packaging plasmid comprises three p5 promoter sequences, a first p5 promoter sequence, a second p5 promoter sequence, and a third p5 promoter sequence, downstream of a coding region.

In some embodiments, a packaging plasmid comprises a linker sequence. In some embodiments, a packaging plasmid comprises a linker sequence between two p5 promoter sequences. In some embodiments, a packaging plasmid comprises a polyadenylation (pA or polyA) sequence downstream of the coding region.

### BRIEF DESCRIPTION OF THE DRAWING

The patent or application file contains at least one drawing executed in color. Copies of this patent or patent application publication with color drawings(s) will be provided by the Office upon request and payment of the necessary fee.

The Figures described below, which together make up the Drawing, are for illustration purposes only, not for limitation.

FIG. 1 shows an exemplary wild-type AAV genome with a single p5 promoter upstream of a coding sequence (e.g., encoding Rep/Cap).

FIG. 2 provides a map of a packaging plasmid in wild-type configuration and its gene products as described herein (blog.addgene.org/viral-vectors-101-parts-of-the-aav-packaging-plasmid).

FIGS. 3A-3D show exemplary packaging plasmid configurations described herein. FIG. 3A demonstrates a p5 promoter configuration used as a benchmark packaging plasmid as described herein. FIG. 3B demonstrates a p5 promoter configuration of one p5 promoter sequence upstream of a coding region and two p5 promoter sequences downstream of a coding region, wherein the two p5 promoter sequences are separated by a linker (e.g., pYZZ233, pYZZ234). FIG. 3C demonstrates a p5 promoter configuration of two p5 promoter sequences downstream of a coding region, wherein the two p5 promoter sequences are separated by a linker (e.g., pYZZ235, pYZZ236, pYZZ408-414). FIG. 3D demonstrates a p5 promoter configuration of three p5 promoter sequences downstream of a coding region, wherein each of the p5 promoter sequences are separated by a linker (e.g., pYZZ415).

FIG. 4 demonstrates titer of rAAV produced using packaging plasmids as described herein.

FIG. 5 demonstrates titer of rAAV produced using packaging plasmids as described herein.

FIG. 6 demonstrates titer of rAAV produced from various cell lines using the packaging plasmids of the present disclosure.

FIG. 7 demonstrates titer of rAAV produced using packaging plasmids as described herein.

FIG. 8 is a bar graph demonstrating the titer of rAAV produced from bioreactors using the packaging plasmids of the present disclosure.

FIG. 9 is a dot plot demonstrating the titer of rAAV produced from bioreactors using the packaging plasmids of the present disclosure.

FIGS. 10A-10C demonstrate features of rAAVs produced using plasmids described herein. FIG. 10A demonstrates genome titer of rAAV achieved using plasmids as described herein. FIG. 10B demonstrates capsid titer of rAAV achieved using plasmids as described herein. FIG. 10C demonstrates percent full rAAV capsids achieved using plasmids as described herein.

FIGS. 11A-11C demonstrate features of rAAVs produced using plasmids described herein. FIG. 11A demonstrates genome titer of rAAV produced from 3 liter bioreactors achieved using the packaging plasmids of the present disclosure. FIG. 11B demonstrates capsid titer of rAAV produced from 3 liter bioreactors achieved using the packaging plasmids of the present disclosure. FIG. 11C depicts the quantification of genome titer and capsid titer demonstrated in FIGS. 13A and 13B.

FIG. 12A demonstrates genome titer of rAAV achieved using plasmids as described herein. FIG. 12B demonstrates capsid titer of rAAV achieved using plasmids as described herein.

FIG. 13A-13B demonstrates rAAV produced drug substance quality achieved using plasmids as described herein. FIG. 13A demonstrates rAAV genome integrity and drug substance relative potency achieved using plasmids as described herein.

FIG. 13B demonstrates purity of rAAV proteins and the percentage of capsids containing genome achieved using plasmids as described herein.

FIG. 14 demonstrates genome titer of rAAV achieved using plasmids as described herein.

FIG. 15A-15B depicts p5 configurations of plasmids as described herein.

FIG. 16A demonstrates genome titer of rAAV achieved using plasmids as described herein. FIG. 16B demonstrates capsid titer of rAAV achieved using plasmids as described herein.

FIG. 17A demonstrates genome titer of rAAV achieved using plasmids as described herein. FIG. 17B demonstrates capsid titer of rAAV achieved using plasmids as described herein.

FIG. 18A demonstrates genome titer of rAAV achieved using plasmids as described herein. FIG. 18B depicts the quantification of genome titer demonstrated in FIG. 18A.

FIG. 19 depicts Rep2Cap9/5/2 plasmid design variations and demonstrates how Cap9/Cap5/Cap2 plasmid names and designs correspond to each other. Plasmids that are underlined have the same rep configuration but different cap genes like Cap9, Cap5, and Cap2.

FIG. 20 demonstrates genome titer of rAAV achieved using plasmids as described herein.

FIG. 21 demonstrates genome titer of rAAV achieved using plasmids as described herein.

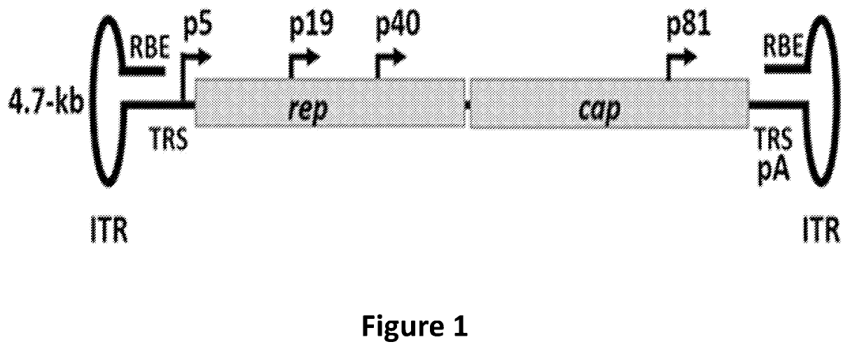

### DEFINITIONS

Adeno-associated virus (AAV): As used herein, the terms “Adeno-associated virus”, “rAAV” and “AAV” refer to viral particles, in whole or in part, of the family Parvoviridae and the genus Dependoparvovirus. AAV is a small replication-defective, nonenveloped virus. AAV includes, but is not limited to, AAV serotype 1, AAV serotype 2, AAV serotype 3 (including serotypes 3A and 3B), AAV serotype 4, AAV serotype 5, AAV serotype 6, AAV serotype 7, AAV serotype 8, AAV serotype 9, AAV serotype 10, AAV serotype 11, AAV serotype 12, AAV serotype 13, snake AAV, avian AAV, bovine AAV, canine AAV, equine AAV, ovine AAV, goat AAV, shrimp AAV, and any variant of any of the foregoing. Wild-type AAV is replication deficient and requires co-infection of cells by a helper virus, e.g., adenovirus, herpes, or vaccinia virus, e.g., an Ad2 or Ad5 virus, or supplementation of helper viral genes, in order to replicate.

Approximately: As used herein, the term “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value)

Downstream: The term “downstream”, as used herein refers to a component, domain, or sequence that exists 3′ relative to another component, domain, or sequence. In some embodiments, the term downstream refers to a nucleotide sequence in relation to a coding region or another nucleotide sequence.

Identity: As used herein, the term “identity” refers to overall relatedness between polymeric molecules, e.g., between nucleic acid molecules (e.g., DNA molecules and/or RNA molecules) and/or between polypeptide molecules. In some embodiments, polymeric molecules are considered to be “substantially identical” to one another if their sequences are at least 25%, 30%, 35%, 40%, 45%, 50%, 55%, 60%, 65%, 70%, 75%, 80%, 85%, 90%, 95%, or 99% identical. Calculation of percent identity of two nucleic acid or polypeptide sequences, for example, can be performed by aligning two sequences for optimal comparison purposes (e.g., gaps can be introduced in one or both of a first and a second sequences for optimal alignment and non-identical sequences can be disregarded for comparison purposes). In some embodiments, a length of a sequence aligned for comparison purposes is at least 30%, at least 40%, at least 50%, at least 60%, at least 70%, at least 80%, at least 90%, at least 95%, or substantially 100% of length of a reference sequence; residues at corresponding positions are then compared. When a position in the first sequence is occupied by the same residue (e.g., nucleotide or amino acid) as a corresponding position in the second sequence, then the two molecules (i.e., first and second) are identical at that position. Percent identity between two sequences is a function of the number of identical positions shared by the two sequences being compared, taking into account the number of gaps, and the length of each gap, which needs to be introduced for optimal alignment of the two sequences. Comparison of sequences and determination of percent identity between two sequences can be accomplished using a mathematical algorithm. For example, percent identity between two nucleotide sequences can be determined using the algorithm of Meyers and Miller (CABIOS, 1989, 4:11-17, which is herein incorporated by reference in its entirety), which has been incorporated into the ALIGN program (version 2.0). In some embodiments, nucleic acid sequence comparisons made with the ALIGN program use a PAM120 weight residue table, a gap length penalty of 12 and a gap penalty of 4.

“Improved,” “increased” or “reduced”: As used herein, these terms, or grammatically comparable comparative terms, indicate values that are relative to a comparable reference measurement. For example, in some embodiments, an assessed value achieved with an agent of interest may be “improved” relative to that obtained with a comparable reference agent. Alternatively or additionally, in some embodiments, an assessed value achieved in a subject or system of interest may be “improved” relative to that obtained in the same subject or system under different conditions (e.g., prior to or after an event such as administration of an agent of interest), or in a different, comparable subject (e.g., in a comparable subject or system that differs from the subject or system of interest in presence of one or more indicators of a particular disease, disorder or condition of interest, or in prior exposure to a condition or agent, etc.). In some embodiments, comparative terms refer to statistically relevant differences (e.g., that are of a prevalence and/or magnitude sufficient to achieve statistical relevance). Those skilled in the art will be aware, or will readily be able to determine, in a given context, a degree and/or prevalence of difference that is required or sufficient to achieve such statistical significance.

Nucleic acid: As used herein, in its broadest sense, the term “nucleic acid” refers to any compound and/or substance that is or can be incorporated into an oligonucleotide chain. In some embodiments, a nucleic acid is a compound and/or substance that is or can be incorporated into an oligonucleotide chain via a phosphodiester linkage. As will be clear from context, in some embodiments, “nucleic acid” refers to an individual nucleic acid residue (e.g., a nucleotide and/or nucleoside); in some embodiments, “nucleic acid” refers to an oligonucleotide chain comprising individual nucleic acid residues. In some embodiments, a “nucleic acid” is or comprises RNA; in some embodiments, a “nucleic acid” is or comprises DNA. In some embodiments, a nucleic acid is, comprises, or consists of one or more natural nucleic acid residues. In some embodiments, a nucleic acid is, comprises, or consists of one or more nucleic acid analogs. In some embodiments, a nucleic acid analog differs from a nucleic acid in that it does not utilize a phosphodiester backbone. For example, in some embodiments, a nucleic acid is, comprises, or consists of one or more “peptide nucleic acids”, which are known in the art and have peptide bonds instead of phosphodiester bonds in the backbone, are considered within the scope of the present invention. Alternatively or additionally, in some embodiments, a nucleic acid has one or more phosphorothioate and/or 5′-N-phosphoramidite linkages rather than phosphodiester bonds. In some embodiments, a nucleic acid is, comprises, or consists of one or more natural nucleosides (e.g., adenosine, thymidine, guanosine, cytidine, uridine, deoxyadenosine, deoxythymidine, deoxy guanosine, and deoxycytidine). In some embodiments, a nucleic acid is, comprises, or consists of one or more nucleoside analogs (e.g., 2-aminoadenosine, 2-thiothymidine, inosine, pyrrolo-pyrimidine, 3-methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, 0 (6)-methylguanine, 2-thiocytidine, methylated bases, intercalated bases, and combinations thereof). In some embodiments, a nucleic acid comprises one or more modified sugars (e.g., 2′-fluororibose, ribose, 2′-deoxyribose, arabinose, and hexose) as compared with those in natural nucleic acids. In some embodiments, a nucleic acid has a nucleotide sequence that encodes a functional gene product such as an RNA or protein. In some embodiments, a nucleic acid includes one or more introns. In some embodiments, nucleic acids are prepared by one or more of isolation from a natural source, enzymatic synthesis by polymerization based on a complementary template (in vivo or in vitro), reproduction in a recombinant cell or system, and chemical synthesis. In some embodiments, a nucleic acid is at least 3, 4, 5, 6, 7, 8, 9, 10, 15, 20, 25, 30, 35, 40, 45, 50, 55, 60, 65, 70, 75, 80, 85, 90, 95, 100, 110, 120, 130, 140, 150, 160, 170, 180, 190, 20, 225, 250, 275, 300, 325, 350, 375, 400, 425, 450, 475, 500, 600, 700, 800, 900, 1000, 1500, 2000, 2500, 3000, 3500, 4000, 4500, 5000 or more residues long. In some embodiments, a nucleic acid is partly or wholly single stranded; in some embodiments, a nucleic acid is partly or wholly double stranded. In some embodiments a nucleic acid has a nucleotide sequence comprising at least one element that encodes, or is the complement of a sequence that encodes, a polypeptide. In some embodiments, a nucleic acid has enzymatic activity.

Protein: As used herein, the term “protein” refers to a polypeptide (i.e., a string of at least two amino acids linked to one another by peptide bonds). Proteins may include moieties other than amino acids (e.g., may be glycoproteins, proteoglycans, etc.) and/or may be otherwise processed or modified. Those of ordinary skill in the art will appreciate that a “protein” can be a complete polypeptide chain as produced by a cell (with or without a signal sequence), or can be a characteristic portion thereof. Those of ordinary skill will appreciate that a protein can sometimes include more than one polypeptide chain, for example linked by one or more disulfide bonds or associated by other means. Polypeptides may contain 1-amino acids, d-amino acids, or both and may contain any of a variety of amino acid modifications or analogs known in the art. Useful modifications include, e.g., terminal acetylation, amidation, methylation, etc. In some embodiments, proteins may comprise natural amino acids, non-natural amino acids, synthetic amino acids, and combinations thereof. The term “peptide” is generally used to refer to a polypeptide having a length of less than about 100 amino acids, less than about 50 amino acids, less than 20 amino acids, or less than 10 amino acids. In some embodiments, proteins are antibodies, antibody fragments, biologically active portions thereof, and/or characteristic portions thereof.

Reference: As used herein describes a standard or control relative to which a comparison is performed. For example, in some embodiments, an agent, animal, individual, population, sample, sequence or value of interest is compared with a reference or control agent, animal, individual, population, sample, sequence or value. In some embodiments, a reference or control is tested and/or determined substantially simultaneously with the testing or determination of interest. In some embodiments, a reference or control is a historical reference or control, optionally embodied in a tangible medium. Typically, as would be understood by those skilled in the art, a reference or control is determined or characterized under comparable conditions or circumstances to those under assessment. Those skilled in the art will appreciate when sufficient similarities are present to justify reliance on and/or comparison to a particular possible reference or control.

Upstream: The term “upstream” when used herein refers to a component, domain, or sequence that exists 5′ relative to another component, domain, or sequence. In some embodiments, the term upstream refers to a nucleotide sequence in relation to a coding region or another nucleotide sequence.

Variant: As used herein in the context of molecules, e.g., nucleic acids, proteins, or small molecules, the term “variant” refers to a molecule that shows significant structural identity with a reference molecule but differs structurally from the reference molecule, e.g., in the presence or absence or in the level of one or more chemical moieties as compared to the reference entity. In some embodiments, a variant also differs functionally from its reference molecule. In general, whether a particular molecule is properly considered to be a “variant” of a reference molecule is based on its degree of structural identity with the reference molecule. As will be appreciated by those skilled in the art, any biological or chemical reference molecule has certain characteristic structural elements. A variant, by definition, is a distinct molecule that shares one or more such characteristic structural elements but differs in at least one aspect from the reference molecule. To give but a few examples, a polypeptide may have a characteristic sequence element comprised of a plurality of amino acids having designated positions relative to one another in linear or three-dimensional space and/or contributing to a particular structural motif and/or biological function; a nucleic acid may have a characteristic sequence element comprised of a plurality of nucleotide residues having designated positions relative to on another in linear or three-dimensional space. In some embodiments, a variant polypeptide or nucleic acid may differ from a reference polypeptide or nucleic acid as a result of one or more differences in amino acid or nucleotide sequence and/or one or more differences in chemical moieties (e.g., carbohydrates, lipids, phosphate groups) that are covalently components of the polypeptide or nucleic acid (e.g., that are attached to the polypeptide or nucleic acid backbone). In some embodiments, a variant polypeptide or nucleic acid shows an overall sequence identity with a reference polypeptide or nucleic acid that is at least 85%, 86%, 87%, 88%, 89%, 90%, 91%, 92%, 93%, 94%, 95%, 96%, 97%, or 99%. In some embodiments, a variant polypeptide or nucleic acid does not share at least one characteristic sequence element with a reference polypeptide or nucleic acid. In some embodiments, a reference polypeptide or nucleic acid has one or more biological activities. In some embodiments, a variant polypeptide or nucleic acid shares one or more of the biological activities of the reference polypeptide or nucleic acid. In some embodiments, a variant polypeptide or nucleic acid lacks one or more of the biological activities of the reference polypeptide or nucleic acid. In some embodiments, a variant polypeptide or nucleic acid shows a reduced level of one or more biological activities as compared to the reference polypeptide or nucleic acid. In some embodiments, a polypeptide or nucleic acid of interest is considered to be a “variant” of a reference polypeptide or nucleic acid if it has an amino acid or nucleotide sequence that is identical to that of the reference but for a small number of sequence alterations at particular positions. Typically, fewer than about 20%, about 15%, about 10%, about 9%, about 8%, about 7%, about 6%, about 5%, about 4%, about 3%, or about 2% of the residues in a variant are substituted, inserted, or deleted, as compared to the reference. In some embodiments, a variant polypeptide or nucleic acid comprises about 10, about 9, about 8, about 7, about 6, about 5, about 4, about 3, about 2, or about 1 substituted residues as compared to a reference. Often, a variant polypeptide or nucleic acid comprises a very small number (e.g., fewer than about 5, about 4, about 3, about 2, or about 1) number of substituted, inserted, or deleted, functional residues (i.e., residues that participate in a particular biological activity) relative to the reference. In some embodiments, a variant polypeptide or nucleic acid comprises not more than about 5, about 4, about 3, about 2, or about 1 addition or deletion, and, in some embodiments, comprises no additions or deletions, as compared to the reference. In some embodiments, a variant polypeptide or nucleic acid comprises fewer than about 25, about 20, about 19, about 18, about 17, about 16, about 15, about 14, about 13, about 10, about 9, about 8, about 7, about 6, and commonly fewer than about 5, about 4, about 3, or about 2 additions or deletions as compared to the reference. In some embodiments, a reference polypeptide or nucleic acid is one found in nature. In some embodiments, a reference polypeptide or nucleic acid is a human polypeptide or nucleic acid.

Vector: As used herein, the term “vector” refers to a nucleic acid molecule capable of transporting another nucleic acid to which it has been linked. One type of vector is a “plasmid”, which refers to a circular double stranded DNA loop into which additional DNA segments may be ligated. Another type of vector is a viral vector, wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “expression vectors.” Standard techniques may be used for recombinant DNA, oligonucleotide synthesis, and tissue culture and transformation (e.g., electroporation, lipofection). Enzymatic reactions and purification techniques may be performed according to manufacturer's specifications or as commonly accomplished in the art or as described herein. The foregoing techniques and procedures may be generally performed according to conventional methods well known in the art and as described in various general and more specific references that are cited and discussed throughout the present specification. See e.g., Sambrook et al., Molecular Cloning: A Laboratory Manual (2d ed., Cold Spring Harbor Laboratory Press, Cold Spring Harbor, N.Y. (1989)), which is incorporated herein by reference for any purpose.

Wild-type: As used herein, the term “wild-type” has its art-understood meaning that refers to an entity having a structure and/or activity as found in nature in a “normal” (as contrasted with mutant, diseased, altered, etc.) state or context. Those of ordinary skill in the art will appreciate that wild-type genes and polypeptides often exist in multiple different forms (e.g., alleles).

Tandem: The term “tandem” when used herein refers to similar or identical components that are in any consecutive order. In some embodiments, the term “tandem” refers to nucleotide sequences that are similar or identical and are in any consecutive order with respect to each other. In some embodiments, these nucleotide sequences will be in consecutive order, but separated by another different nucleotide sequence(s). In some embodiments, tandem refers to two or more of the same nucleotide sequences separated by a different nucleotide sequence(s). In some embodiments, tandem refers to identical or nearly identical nucleotide sequences that are adjacent to each other.

### DETAILED DESCRIPTION OF CERTAIN EMBODIMENTS

rAAV

In general terms, to allow for production of rAAV, a host cell is provided with AAV ITRs flanking an expression cassette, AAV rep and cap gene functions, as well as additional helper functions. These may be provided to the cell using any number of appropriate plasmids or vectors and/or via integration into the host cell genome. Additional helper functions can be provided by, for example, an adenovirus infection, by an adenoviral helper plasmid that carries all of the required adenoviral helper function genes, or by other viruses such as HSV. Any genes, gene functions, or genetic material necessary for rAAV production by the cell may transiently exist within the cell, or be stably inserted into the cell genome. It is to be understood that any suitable host cell and vector/plasmid/virus may be used in any such method for production of rAAV.

In some embodiments, rAAV vectors are produced by transfection of a host cell. In some embodiments, the host cell is transfected with at least a packaging plasmid. In some embodiments, the host cell is transfected with at least an adenoviral helper plasmid. In some embodiments, the host cell is transfected with at least a proviral plasmid. In some embodiments, the host cell is transfected with at least a packaging plasmid and an adenoviral helper plasmid. In some embodiments, the host cell is transfected with at least a packaging plasmid and a proviral plasmid. In some embodiments, the host cell is transfected with at least an adenoviral helper plasmid and a proviral plasmid. In some embodiments, rAAV vectors are produced by transfection of a host cell with a packaging plasmid, an adenoviral helper plasmid, and a proviral plasmid. It is to be understood that the present disclosure also encompasses methods where one or more of these plasmids are combined into a single plasmid, e.g., where the packaging and adenoviral helper plasmids are combined into a single packaging/helper plasmid and a dual transfection with a proviral plasmid is used instead of a traditional triple transfection. In some embodiments, a proviral plasmid is a gene of interest (GOI) plasmid and/or contains a gene of interest. In some embodiments, a dual transfection comprises a single packaging/helper plasmid and another plasmid useful for rAAV production. In some embodiments, a single packaging/helper plasmid comprises sequences in addition to packaging plasmid and adenoviral helper sequences.

In some embodiments, rAAV vectors are produced by infection of a host cell. In some embodiments, the host cell is infected with a helper virus (e.g., an adenovirus or herpes simplex virus), which allows the rAAV to replicate.

In some embodiments, an adenoviral helper plasmid comprises nucleic acid sequences encoding adenovirus replication proteins. In some embodiments, an adenoviral helper plasmid comprises nucleic acid sequences encoding, for example, E4orf6, E2a and/or VA RNA. In some embodiments, a host cell used for rAAV production has nucleic acid sequences encoding adenovirus replication proteins integrated in the genome and the method of rAAV production does not involve transfection with an adenoviral helper plasmid.

In some embodiments, a proviral plasmid comprises nucleic acid sequences encoding a payload (e.g., a cDNA expression cassette for a transgene of interest) flanked by AAV inverted terminal repeats (ITRs). In some embodiments, a proviral plasmid further comprises nucleic acid sequences encoding regulatory sequences, e.g., promoters, introns, enhancers, etc. to regulate expression of the payload in the cells or tissue of interest. In some embodiments, a host cell used for rAAV production has nucleic acid sequences encoding the payload flanked by AAV inverted terminal repeats (ITRs) integrated in the genome and the method of rAAV production does not involve transfection with a proviral plasmid. In some embodiments, a host cell used for rAAV production has nucleic acid sequences encoding adenovirus replication proteins and nucleic acid sequences encoding the payload flanked by AAV inverted terminal repeats (ITRs) integrated in the genome and the method of rAAV production does not involve transfection with an adenoviral helper plasmid or a proviral plasmid.

In some embodiments, a packaging plasmid, among other things, comprises a coding region. In some embodiments, a coding region of a packaging plasmid comprises nucleic acid sequences encoding AAV Rep and Cap genes. In some embodiments, a packaging plasmid, as described herein has increased expression of the Rep and Cap genes of the coding region relative to a control. In some embodiments, a control is a wild-type packaging plasmid (e.g., a packaging plasmid not containing a p5 sequence downstream of the coding region). In some embodiments, a Rep gene encodes for the Rep78, Rep68, Rep52, and Rep40 proteins. In some embodiments, a Cap gene encodes for VP1, VP2, and VP3 proteins which are essential for AAV capsid formation. In some embodiments, a packaging plasmid of the present disclosure may further comprise adenoviral helper function genes.

The present disclosure provides, among other things, rAAVs produced using methods described herein. Generally, rAAVs produced using methods described herein may be of any AAV serotype. AAV serotypes generally have different tropisms to infect different cells or tissues. In some embodiments, an AAV serotype is selected based on a tropism for a particular cell type or tissue type.

In some embodiments, an rAAV may comprise or be based on a serotype selected from any of the following serotypes, and variants thereof, including, but not limited to: AAV1, AAV10, AAV106.1/hu.37, AAV11, AAV114.3/hu.40, AAV 12, AAV127.2/hu.41, AAV127.5/hu.42, AAV128.1/hu.43, AAV128.3/hu.44, AAV130.4/hu.48, AAV145.1/hu.53, AAV145.5/hu.54, AAV145.6/hu.55, AAV16.12/hu.11, AAV16.3, AAV16.8/hu.10, AAV161.10/hu.60, AAV161.6/hu.61, AAV1-7/rh.48, AAV1-8/rh.49, AAV2, AAV2.5T, AAV2-15/rh.62, AAV223.1, AAV223.2, AAV223.4, AAV223.5, AAV223.6, AAV223.7, AAV2-3/rh.61, AAV24.1, AAV2-4/rh.50, AAV2-5/rh.51, AAV27.3, AAV29.3/bb.1, AAV29.5/bb.2, AAV2G9, AAV-2-pre-miRNA-101, AAV3A, AAV3B, AAV3.1/hu.6, AAV3.1/hu.9, AAV3-11/rh.53, AAV3-3, AAV33.12/hu.17, AAV33.4/hu.15, AAV33.8/hu.16, AAV3-9/rh.52, AAV3a, AAV3b, AAV4, AAV4-19/rh.55, AAV42.12, AAV42-10, AAV42-11, AAV42-12, AAV42-13, AAV42-15, AAV42-1b, AAV42-2, AAV42-3a, AAV42-3b, AAV42-4, AAV42-5a, AAV42-5b, AAV42-6b, AAV42-8, AAV42-aa, AAV43-1, AAV43-12, AAV43-20, AAV43-21, AAV43-23, AAV43-25, AAV43-5, AAV4-4, AAV44.1, AAV44.2, AAV44.5, AAV46.2/hu.28, AAV46.6/hu.29, AAV4-8/r 11.64, AAV4-8/rh.64, AAV4-9/rh.54, AAV5, AAV52.1/hu.20, AAV52/hu.19, AAV5-22/rh.58, AAV5-3/rh.57, AAV54.1/hu.21, AAV54.2/hu.22, AAV54.4R/hu.27, AAV54.5/hu.23, AAV54.7/hu.24, AAV58.2/hu.25, AAV6, AAV6.1, AAV6.1.2, AAV6.2, AAV7, AAV7.2, AAV7.3/hu.7, AAV8, AAV-8b, AAV-8h, AAV9, AAV9.11, AAV9.13, AAV9.16, AAV9.24, AAV9.45, AAV9.47, AAV9.61, AAV9.68, AAV9.84, AAV9.9, AAVA3.3, AAVA3.4, AAVA3.5, AAVA3.7, AAV-b, AAVC1, AAVC2, AAVC5, AAVCh.5, AAVCh.5R1, AAVcy.2, AAVcy.3, AAVcy.4, AAVcy.5, AAVCy.5R1, AAVCy.5R2, AAVCy.5R3, AAVCy.5R4, AAVcy.6, AAV-DJ, AAV-DJ8, AAVF3, AAVF5, AAV-h, AAVH-1/hu.1, AAVH2, AAVH-5/hu.3, AAVH6, AAVhE1.1, AAVhER1.14, AAVhEr1.16, AAVhEr1.18, AAVhER1.23, AAVhEr1.35, AAVhEr1.36, AAVhEr1.5, AAVhEr1.7, AAVhEr1.8, AAVhEr2.16, AAVhEr2.29, AAVhEr2.30, AAVhEr2.31, AAVhEr2.36, AAVhEr2.4, AAVhEr3.1, AAVhu.1, AAVhu.10, AAVhu.11, AAVhu.12, AAVhu.13, AAVhu.14/9, AAVhu.15, AAVhu.16, AAVhu.17, AAVhu.18, AAVhu.19, AAVhu.2, AAVhu.20, AAVhu.21, AAVhu.22, AAVhu.23.2, AAVhu.24, AAVhu.25, AAVhu.27, AAVhu.28, AAVhu.29, AAVhu.29R, AAVhu.3, AAVhu.31, AAVhu.32, AAVhu.34, AAVhu.35, AAVhu.37, AAVhu.39, AAVhu.4, AAVhu.40, AAVhu.41, AAVhu.42, AAVhu.43, AAVhu.44, AAVhu.44R1, AAVhu.44R2, AAVhu.44R3, AAVhu.45, AAVhu.46, AAVhu.47, AAVhu.48, AAVhu.48R1, AAVhu.48R2, AAVhu.48R3, AAVhu.49, AAVhu.5, AAVhu.51, AAVhu.52, AAVhu.53, AAVhu.54, AAVhu.55, AAVhu.56, AAVhu.57, AAVhu.58, AAVhu.6, AAVhu.60, AAVhu.61, AAVhu.63, AAVhu.64, AAVhu.66, AAVhu.67, AAVhu.68, AAVhu.7, AAVhu.8, AAVhu.9, AAVhu.t 19, AAVLG-10/rh.40, AAVLG-4/rh.38, AAVLG-9/hu.39, AAVLG-9/hu.39, AAV-LK01, AAV-LK02, AAVLK03, AAV-LK03, AAV-LK04, AAV-LK05, AAV-LK06, AAV-LK07, AAV-LK08, AAV-LK09, AAV-LK10, AAV-LK11, AAV-LK12, AAV-LK13, AAV-LK14, AAV-LK15, AAV-LK17, AAV-LK18, AAV-LK19, AAVN721-8/rh.43, AAV-PAEC, AAV-PAEC11, AAV-PAEC12, AAV-PAEC2, AAV-PAEC4, AAV-PAEC6, AAV-PAEC7, AAV-PAEC8, AAVpi.1, AAVpi.2, AAVpi.3, AAVrh.10, AAVrh.12, AAVrh.13, AAVrh.13R, AAVrh.14, AAVrh.17, AAVrh.18, AA Vrh.19, AAVrh.2, AAVrh.20, AAVrh.21, AAVrh.22, AAVrh.23, AAVrh.24, AAVrh.25, AAVrh.2R, AAVrh.31, AAVrh.32, AAVrh.33, AAVrh.34, AAVrh.35, AAVrh.36, AAVrh.37, AAVrh.37R2, AAVrh.38, AAVrh.39, AAVrh.40, AAVrh.43, AAVrh.44, AAVrh.45, AAVrh.46, AAVrh.47, AAVrh.48, AAVrh.48, AAVrh.48.1, AAVrh.48.1.2, AAVrh.48.2, AAVrh.49, AAVrh.50, AAVrh.51, AAVrh.52, AA Vrh.53, AAVrh.54, AAVrh.55, AAVrh.56, AAVrh.57, AAVrh.58, AAVrh.59, AAVrh.60, AAVrh.61, AAVrh.62, AAVrh.64, AAVrh.64R1, AAVrh.64R2, AAVrh.65, AAVrh.67, AAVrh.68, AAVrh.69, AAVrh.70, AAVrh.72, AAVrh.73, AAVrh.74, AAVrh.8, AAVrh.8R, AAVrh8R, AAVrh8R A586R mutant, AAVrh8R R533A mutant, BAAV, B P61 AAV, B P62 AAV, B P63AAV, bovine AAV, caprine AAV, Japanese AAV10, true type AAV (ttAAV), UPENN AAV 10, AAV-LK 16, AAAV, AAV Shuffle 100-1, AAV Shuffle 100-2, AAV Shuffle 100-3, AAV Shuffle 100-7, AAV Shuffle 10-2, AAV Shuffle 10-6, AAV Shuffle 10-8, AAV SM 100-10, AAV SM 100-3, AAV SM 10-1, AAV SM 10-2, AAV SM 10-8, AAV-PHP.B, AAV-PHP.N, AAV-PHP.S, AAVrh.74, AAV-HSC 1-17, AAV-CBr, AAV-CLv, AAV-CLg, and/or AAV.CAP-B1 to AAV.CAP-B25.

In certain embodiments, an rAAV comprises an AAV2 serotype or a variant thereof. In certain embodiments, an rAAV comprises an AAV5 serotype or a variant thereof. In certain embodiments, an rAAV comprises an AAV8 serotype or a variant thereof. In certain embodiments, an rAAV comprises an AAV9 serotype or a variant thereof. In certain embodiments, an rAAV comprises AAVhu.68 serotype or a variant thereof. In certain embodiments, an rAAV comprises AAVrh.10 serotype or a variant thereof.

In some embodiments, a replication sequence is any one of SEQ ID NO: 1-2 and capsid sequence is SEQ ID NO: 3. In some embodiments, a replication sequence is any one of SEQ ID NO: 1-2 and capsid sequence is SEQ ID NO: 50. In some embodiments, a replication sequence is any one of SEQ ID NO: 1-2 and capsid sequence is SEQ ID NO: 51.

TABLE 1
Exemplary replication and capsid sequences

| SEQ ID NO: | Name | Sequence |
|---|---|---|
| 1 | Replication 2 | ATGCCGGGGTTTTACGAGATTGTGATTAAGGTCCCCAGCGACCTTGACGAGC |
| | | ATCTGCCCGGCATTTCTGACAGCTTTGTGAACTGGGTGGCCGAGAAGGAATG |
| | | GGAGTTGCCGCCAGATTCTGACATGGATCTGAATCTGATTGAGCAGGCACCC |
| | | CTGACCGTGGCCGAGAAGCTGCAGCGCGACTTTCTGACGGAATGGCGCCGTG |
| | | TGAGTAAGGCCCCGGAGGCTCTTTTCTTTGTGCAATTTGAGAAGGGAGAGAG |
| | | CTACTTCCACATGCACGTGCTCGTGGAAACCACCGGGGTGAAATCCATGGTT |
| | | TTGGGACGTTTCCTGAGTCAGATTCGCGAAAAACTGATTCAGAGAATTTACC |
| | | GCGGGATCGAGCCGACTTTGCCAAACTGGTTCGCGGTCACAAAGACCAGAAA |
| | | TGGCGCCGGAGGCGGGAACAAGGTGGTGGATGAGTGCTACATCCCCAATTAC |
| | | TTGCTCCCCAAAACCCAGCCTGAGCTCCAGTGGGCGTGGACTAATATGGAAC |
| | | AGTATTTAAGCGCCTGTTTGAATCTCACGGAGCGTAAACGGTTGGTGGCGCA |
| | | GCATCTGACGCACGTGTCGCAGACGCAGGAGCAGAACAAAGAGAATCAGAAT |
| | | CCCAATTCTGATGCGCCGGTGATCAGATCAAAAACTTCAGCCAGGTACATGG |
| | | AGCTGGTCGGGTGGCTCGTGGACAAGGGGATTACCTCGGAGAAGCAGTGGAT |
| | | CCAGGAGGACCAGGCCTCATACATCTCCTTCAATGCGGCCTCCAACTCGCGG |
| | | TCCCAAATCAAGGCTGCCTTGGACAATGCGGGAAAGATTATGAGCCTGACTA |
| | | AAACCGCCCCCGACTACCTGGTGGGCCAGCAGCCCGTGGAGGACATTTCCAG |
| | | CAATCGGATTTATAAAATTTTGGAACTAAACGGGTACGATCCCCAATATGCG |
| | | GCTTCCGTCTTTCTGGGATGGGCCACGAAAAAGTTCGGCAAGAGGAACACCA |
| | | TCTGGCTGTTTGGGCCTGCAACTACCGGGAAGACCAACATCGCGGAGGCCAT |
| | | AGCCCACACTGTGCCCTTCTACGGGTGCGTAAACTGGACCAATGAGAACTTT |
| | | CCCTTCAACGACTGTGTCGACAAGATGGTGATCTGGTGGGAGGAGGGGAAGA |
| | | TGACCGCCAAGGTCGTGGAGTCGGCCAAAGCCATTCTCGGAGGAAGCAAGGT |
| | | GCGCGTGGACCAGAAATGCAAGTCCTCGGCCCAGATAGACCCGACTCCCGTG |
| | | ATCGTCACCTCCAACACCAACATGTGCGCCGTGATTGACGGGAACTCAACGA |
| | | CCTTCGAACACCAGCAGCCGTTGCAAGACCGGATGTTCAAATTTGAACTCAC |
| | | CCGCCGTCTGGATCATGACTTTGGGAAGGTCACCAAGCAGGAAGTCAAAGAC |
| | | TTTTTCCGGTGGGCAAAGGATCACGTGGTTGAGGTGGAGCATGAATTCTACG |
| | | TCAAAAAGGGTGGAGCCAAGAAAAGACCCGCCCCCAGTGACGCAGATATAAG |
| | | TGAGCCCAAACGGGTGCGCGAGTCAGTTGCGCAGCCATCGACGTCAGACGCG |
| | | GAAGCTTCGATCAACTACGCAGACAGGTACCAAAACAAATGTTCTCGTCACG |
| | | TGGGCATGAATCTGATGCTGTTTCCCTGCAGACAATGCGAGAGAATGAATCA |
| | | GAATTCAAATATCTGCTTCACTCACGGACAGAAAGACTGTTTAGAGTGCTTT |
| | | CCCGTGTCAGAATCTCAACCCGTTTCTGTCGTCAAAAAGGCGTATCAGAAAC |
| | | TGTGCTACATTCATCATATCATGGGAAAGGTGCCAGACGCTTGCACTGCCTG |
| | | CGATCTGGTCAATGTGGATTTGGATGACTGCATCTTTGAACAATAA |
| 2 | Alternative | ACGCCGGGGTTTTACGAGATTGTGATTAAGGTCCCCAGCGACCTTGACGAGC |
| | replication 2 | ATCTGCCCGGCATTTCTGACAGCTTTGTGAACTGGGTGGCCGAGAAGGAATG |
| | | GGAGTTGCCGCCAGATTCTGACATGGATCTGAATCTGATTGAGCAGGCACCC |
| | | CTGACCGTGGCCGAGAAGCTGCAGCGCGACTTTCTGACGGAATGGCGCCGTG |
| | | TGAGTAAGGCCCCGGAGGCTCTTTTCTTTGTGCAATTTGAGAAGGGAGAGAG |
| | | CTACTTCCACATGCACGTGCTCGTGGAAACCACCGGGGTGAAATCCATGGTT |
| | | TTGGGACGTTTCCTGAGTCAGATTCGCGAAAAACTGATTCAGAGAATTTACC |
| | | GCGGGATCGAGCCGACTTTGCCAAACTGGTTCGCGGTCACAAAGACCAGAAA |
| | | TGGCGCCGGAGGCGGGAACAAGGTGGTGGATGAGTGCTACATCCCCAATTAC |
| | | TTGCTCCCCAAAACCCAGCCTGAGCTCCAGTGGGCGTGGACTAATATGGAAC |
| | | AGTATTTAAGCGCCTGTTTGAATCTCACGGAGCGTAAACGGTTGGTGGCGCA |
| | | GCATCTGACGCACGTGTCGCAGACGCAGGAGCAGAACAAAGAGAATCAGAAT |
| | | CCCAATTCTGATGCGCCGGTGATCAGATCAAAAACTTCAGCCAGGTACATGG |
| | | AGCTGGTCGGGTGGCTCGTGGACAAGGGGATTACCTCGGAGAAGCAGTGGAT |
| | | CCAGGAGGACCAGGCCTCATACATCTCCTTCAATGCGGCCTCCAACTCGCGG |
| | | TCCCAAATCAAGGCTGCCTTGGACAATGCGGGAAAGATTATGAGCCTGACTA |
| | | AAACCGCCCCCGACTACCTGGTGGGCCAGCAGCCCGTGGAGGACATTTCCAG |
| | | CAATCGGATTTATAAAATTTTGGAACTAAACGGGTACGATCCCCAATATGCG |
| | | GCTTCCGTCTTTCTGGGATGGGCCACGAAAAAGTTCGGCAAGAGGAACACCA |
| | | TCTGGCTGTTTGGGCCTGCAACTACCGGGAAGACCAACATCGCGGAGGCCAT |
| | | AGCCCACACTGTGCCCTTCTACGGGTGCGTAAACTGGACCAATGAGAACTTT |
| | | CCCTTCAACGACTGTGTCGACAAGATGGTGATCTGGTGGGAGGAGGGGAAGA |
| | | TGACCGCCAAGGTCGTGGAGTCGGCCAAAGCCATTCTCGGAGGAAGCAAGGT |
| | | GCGCGTGGACCAGAAATGCAAGTCCTCGGCCCAGATAGACCCGACTCCCGTG |
| | | ATCGTCACCTCCAACACCAACATGTGCGCCGTGATTGACGGGAACTCAACGA |
| | | CCTTCGAACACCAGCAGCCGTTGCAAGACCGGATGTTCAAATTTGAACTCAC |
| | | CCGCCGTCTGGATCATGACTTTGGGAAGGTCACCAAGCAGGAAGTCAAAGAC |
| | | TTTTTCCGGTGGGCAAAGGATCACGTGGTTGAGGTGGAGCATGAATTCTACG |
| | | TCAAAAAGGGTGGAGCCAAGAAAAGACCCGCCCCCAGTGACGCAGATATAAG |
| | | TGAGCCCAAACGGGTGCGCGAGTCAGTTGCGCAGCCATCGACGTCAGACGCG |
| | | GAAGCTTCGATCAACTACGCAGACAGGTACCAAAACAAATGTTCTCGTCACG |
| | | TGGGCATGAATCTGATGCTGTTTCCCTGCAGACAATGCGAGAGAATGAATCA |
| | | GAATTCAAATATCTGCTTCACTCACGGACAGAAAGACTGTTTAGAGTGCTTT |
| | | CCCGTGTCAGAATCTCAACCCGTTTCTGTCGTCAAAAAGGCGTATCAGAAAC |
| | | TGTGCTACATTCATCATATCATGGGAAAGGTGCCAGACGCTTGCACTGCCTG |
| | | CGATCTGGTCAATGTGGATTTGGATGACTGCATCTTTGAACAATAA |
| 3 | Capsid 9 | ATGGCTGCCGATGGTTATCTTCCAGATTGGCTCGAGGACAACCTTAGTGAAG |
| | | GAATTCGCGAGTGGTGGGCTTTGAAACCTGGAGCCCCTCAACCCAAGGCAAA |
| | | TCAACAACATCAAGACAACGCTCGAGGTCTTGTGCTTCCGGGTTACAAATAC |
| | | CTTGGACCCGGCAACGGACTCGACAAGGGGGAGCCGGTCAACGCAGCAGACG |
| | | CGGCGGCCCTCGAGCACGACAAGGCCTACGACCAGCAGCTCAAGGCCGGAGA |
| | | CAACCCGTACCTCAAGTACAACCACGCCGACGCCGAGTTCCAGGAGCGGCTC |
| | | AAAGAAGATACGTCTTTTGGGGGCAACCTCGGGCGAGCAGTCTTCCAGGCCA |
| | | AAAAGAGGCTTCTTGAACCTCTTGGTCTGGTTGAGGAAGCGGCTAAGACGGC |
| | | TCCTGGAAAGAAGAGGCCTGTAGAGCAGTCTCCTCAGGAACCGGACTCCTCC |
| | | GCGGGTATTGGCAAATCGGGTGCACAGCCCGCTAAAAAGAGACTCAATTTCG |
| | | GTCAGACTGGCGACACAGAGTCAGTCCCAGACCCTCAACCAATCGGAGAACC |
| | | TCCCGCAGCCCCCTCAGGTGTGGGATCTCTTACAATGGCTTCAGGTGGTGGC |
| | | GCACCAGTGGCAGACAATAACGAAGGTGCCGATGGAGTGGGTAGTTCCTCGG |
| | | GAAATTGGCATTGCGATTCCCAATGGCTGGGGGACAGAGTCATCACCACCAG |
| | | CACCCGAACCTGGGCCCTGCCCACCTACAACAATCACCTCTACAAGCAAATC |
| | | TCCAACAGCACATCTGGAGGATCTTCAAATGACAACGCCTACTTCGGCTACA |
| | | GCACCCCCTGGGGGTATTTTGACTTCAACAGATTCCACTGCCACTTCTCACC |
| | | ACGTGACTGGCAGCGACTCATCAACAACAACTGGGGATTCCGGCCTAAGCGA |
| | | CTCAACTTCAAGCTCTTCAACATTCAGGTCAAAGAGGTTACGGACAACAATG |
| | | GAGTCAAGACCATCGCCAATAACCTTACCAGCACGGTCCAGGTCTTCACGGA |
| | | CTCAGACTATCAGCTCCCGTACGTGCTCGGGTCGGCTCACGAGGGCTGCCTC |
| | | CCGCCGTTCCCAGCGGACGTTTTCATGATTCCTCAGTACGGGTATCTGACGC |
| | | TTAATGATGGAAGCCAGGCCGTGGGTCGTTCGTCCTTTTACTGCCTGGAATA |
| | | TTTCCCGTCGCAAATGCTAAGAACGGGTAACAACTTCCAGTTCAGCTACGAG |
| | | TTTGAGAACGTACCTTTCCATAGCAGCTACGCTCACAGCCAAAGCCTGGACC |
| | | GACTAATGAATCCACTCATCGACCAATACTTGTACTATCTCTCAAAGACTAT |
| | | TAACGGTTCTGGACAGAATCAACAAACGCTAAAATTCAGTGTGGCCGGACCC |
| | | AGCAACATGGCTGTCCAGGGAAGAAACTACATACCTGGACCCAGCTACCGAC |
| | | AACAACGTGTCTCAACCACTGTGACTCAAAACAACAACAGCGAATTTGCTTG |
| | | GCCTGGAGCTTCTTCTTGGGCTCTCAATGGACGTAATAGCTTGATGAATCCT |
| | | GGACCTGCTATGGCCAGCCACAAAGAAGGAGAGGACCGTTTCTTTCCTTTGT |
| | | CTGGATCTTTAATTTTTGGCAAACAAGGAACTGGAAGAGACAACGTGGATGC |
| | | GGACAAAGTCATGATAACCAACGAAGAAGAAATTAAAACTACTAACCCGGTA |
| | | GCAACGGAGTCCTATGGACAAGTGGCCACAAACCACCAGAGTGCCCAAGCAC |
| | | AGGCGCAGACCGGCTGGGTTCAAAACCAAGGAATACTTCCGGGTATGGTTTG |
| | | GCAGGACAGAGATGTGTACCTGCAAGGACCCATTTGGGCCAAAATTCCTCAC |
| | | ACGGACGGCAACTTTCACCCTTCTCCGCTGATGGGAGGGTTTGGAATGAAGC |
| | | ACCCGCCTCCTCAGATCCTCATCAAAAACACACCTGTACCTGCGGATCCTCC |
| | | AACGGCCTTCAACAAGGACAAGCTGAACTCTTTCATCACCCAGTATTCTACT |
| | | GGCCAAGTCAGCGTGGAGATCGAGTGGGAGCTGCAGAAGGAAAACAGCAAGC |
| | | GCTGGAACCCGGAGATCCAGTACACTTCCAACTATTACAAGTCTAATAATGT |
| | | TGAATTTGCTGTTAATACTGAAGGTGTATATAGTGAACCCCGCCCCATTGGC |
| | | ACCAGATACCTGACTCGTAATCTGTAA |
| 50 | Capsid 2 | ATGGCTGCCGATGGTTATCTTCCAGATTGGCTCGAGGACACTCTCTCTGAAG |
| | | GAATAAGACAGTGGTGGAAGCTCAAACCTGGCCCACCACCACCAAAGCCCGC |
| | | AGAGCGGCATAAGGACGACAGCAGGGGTCTTGTGCTTCCTGGGTACAAGTAC |
| | | CTCGGACCCTTCAACGGACTCGACAAGGGAGAGCCGGTCAACGAGGCAGACG |
| | | CCGCGGCCCTCGAGCACGACAAAGCCTACGACCGGCAGCTCGACAGCGGAGA |
| | | CAACCCGTACCTCAAGTACAACCACGCCGACGCGGAGTTTCAGGAGCGCCTT |
| | | AAAGAAGATACGTCTTTTGGGGGCAACCTCGGACGAGCAGTCTTCCAGGCGA |
| | | AAAAGAGGGTTCTTGAACCTCTGGGCCTGGTTGAGGAACCTGTTAAGACGGC |
| | | TCCGGGAAAAAAGAGGCCGGTAGAGCACTCTCCTGTGGAGCCAGACTCCTCC |
| | | TCGGGAACCGGAAAGGCGGGCCAGCAGCCTGCAAGAAAAAGATTGAATTTTG |
| | | GTCAGACTGGAGACGCAGACTCAGTACCTGACCCCCAGCCTCTCGGACAGCC |
| | | ACCAGCAGCCCCCTCTGGTCTGGGAACTAATACGATGGCTACAGGCAGTGGC |
| | | GCACCAATGGCAGACAATAACGAGGGCGCCGACGGAGTGGGTAATTCCTCGG |
| | | GAAATTGGCATTGCGATTCCACATGGATGGGCGACAGAGTCATCACCACCAG |
| | | CACCCGAACCTGGGCCCTGCCCACCTACAACAACCACCTCTACAAACAAATT |
| | | TCCAGCCAATCAGGAGCCTCGAACGACAATCACTACTTTGGCTACAGCACCC |
| | | CTTGGGGGTATTTTGACTTCAACAGATTCCACTGCCACTTTTCACCACGTGA |
| | | CTGGCAAAGACTCATCAACAACAACTGGGGATTCCGACCCAAGAGACTCAAC |
| | | TTCAAGCTCTTTAACATTCAAGTCAAAGAGGTCACGCAGAATGACGGTACGA |
| | | CGACGATTGCCAATAACCTTACCAGCACGGTTCAGGTGTTTACTGACTCGGA |
| | | GTACCAGCTCCCGTACGTCCTCGGCTCGGCGCATCAAGGATGCCTCCCGCCG |
| | | TTCCCAGCAGACGTCTTCATGGTGCCACAGTATGGATACCTCACCCTGAACA |
| | | ACGGGAGTCAGGCAGTAGGACGCTCTTCATTTTACTGCCTGGAGTACTTTCC |
| | | TTCTCAGATGCTGCGTACCGGAAACAACTTTACCTTCAGCTACACTTTTGAG |
| | | GACGTTCCTTTCCACAGCAGCTACGCTCACAGCCAGAGTCTGGACCGTCTCA |
| | | TGAATCCTCTCATCGACCAGTACCTGTATTACTTGAGCAGAACAAACACTCC |
| | | AAGTGGAACCACCACGCAGTCAAGGCTTCAGTTTTCTCAGGCCGGAGCGAGT |
| | | GACATTCGGGACCAGTCTAGGAACTGGCTTCCTGGACCCTGTTACCGCCAGC |
| | | AGCGAGTATCAAAGACATCTGCGGATAACAACAACAGTGAATACTCGTGGAC |
| | | TGGAGCTACCAAGTACCACCTCAATGGCAGAGACTCTCTGGTGAATCCGGGC |
| | | CCGGCCATGGCAAGCCACAAGGACGATGAAGAAAAGTTTTTTCCTCAGAGCG |
| | | GGGTTCTCATCTTTGGGAAGCAAGGCTCAGAGAAAACAAATGTGGACATTGA |
| | | AAAGGTCATGATTACAGACGAAGAGGAAATCAGGACAACCAATCCCGTGGCT |
| | | ACGGAGCAGTATGGTTCTGTATCTACCAACCTCCAGAGAGGCAACAGACAAG |
| | | CAGCTACCGCAGATGTCAACACACAAGGCGTTCTTCCAGGCATGGTCTGGCA |
| | | GGACAGAGATGTGTACCTTCAGGGGCCCATCTGGGCAAAGATTCCACACACG |
| | | GACGGACATTTTCACCCCTCTCCCCTCATGGGTGGATTCGGACTTAAACACC |
| | | CTCCTCCACAGATTCTCATCAAGAACACCCCGGTACCTGCGAATCCTTCGAC |
| | | CACCTTCAGTGCGGCAAAGTTTGCTTCCTTCATCACACAGTACTCCACGGGA |
| | | CAGGTCAGCGTGGAGATCGAGTGGGAGCTGCAGAAGGAAAACAGCAAACGCT |
| | | GGAATCCCGAAATTCAGTACACTTCCAACTACAACAAGTCTGTTAATGTGGA |
| | | CTTTACTGTGGACACTAATGGCGTGTATTCAGAGCCTCGCCCCATTGGCACC |
| | | AGATACCTGACTCGTAATCTGTAA |
| 51 | Capsid 5 | ATGTCTTTTGTTGATCACCCTCCAGATTGGTTGGAAGAAGTTGGTGAAGGTC |
| | | TTCGCGAGTTTTTGGGCCTTGAAGCGGGCCCACCGAAACCAAAACCCAATCA |
| | | GCAGCATCAAGATCAAGCCCGTGGTCTTGTGCTGCCTGGTTATAACTATCTC |
| | | GGACCCGGAAACGGTCTCGATCGAGGAGAGCCTGTCAACAGGGCAGACGAGG |
| | | TCGCGCGAGAGCACGACATCTCGTACAACGAGCAGCTTGAGGCGGGAGACAA |
| | | CCCCTACCTCAAGTACAACCACGCGGACGCCGAGTTTCAGGAGAAGCTCGCC |
| | | GACGACACATCCTTCGGGGGAAACCTCGGAAAGGCAGTCTTTCAGGCCAAGA |
| | | AAAGGGTTCTCGAACCTTTTGGCCTGGTTGAAGAGGGTGCTAAGACGGCCCC |
| | | TACCGGAAAGCGGATAGACGACCACTTTCCAAAAAGAAAGAAGGCTCGGACC |
| | | GAAGAGGACTCCAAGCCTTCCACCTCGTCAGACGCCGAAGCTGGACCCAGCG |
| | | GATCCCAGCAGCTGCAAATCCCAGCCCAACCAGCCTCAAGTTTGGGAGCTGA |
| | | TACAATGTCTGCGGGAGGTGGCGGCCCATTGGGCGACAATAACCAAGGTGCC |
| | | GATGGAGTGGGCAATGCCTCGGGAGATTGGCATTGCGATTCCACGTGGATGG |
| | | GGGACAGAGTCGTCACCAAGTCCACCCGAACCTGGGTGCTGCCCAGCTACAA |
| | | CAACCACCAGTACCGAGAGATCAAAAGCGGCTCCGTCGACGGAAGCAACGCC |
| | | AACGCCTACTTTGGATACAGCACCCCCTGGGGGTACTTTGACTTTAACCGCT |
| | | TCCACAGCCACTGGAGCCCCCGAGACTGGCAAAGACTCATCAACAACTACTG |
| | | GGGCTTCAGACCCCGGTCCCTCAGAGTCAAAATCTTCAACATTCAAGTCAAA |
| | | GAGGTCACGGTGCAGGACTCCACCACCACCATCGCCAACAACCTCACCTCCA |
| | | CCGTCCAAGTGTTTACGGACGACGACTACCAGCTGCCCTACGTCGTCGGCAA |
| | | CGGGACCGAGGGATGCCTGCCGGCCTTCCCTCCGCAGGTCTTTACGCTGCCG |
| | | CAGTACGGTTACGCGACGCTGAACCGCGACAACACAGAAAATCCCACCGAGA |
| | | GGAGCAGCTTCTTCTGCCTAGAGTACTTTCCCAGCAAGATGCTGAGAACGGG |
| | | CAACAACTTTGAGTTTACCTACAACTTTGAGGAGGTGCCCTTCCACTCCAGC |
| | | TTCGCTCCCAGTCAGAACCTGTTCAAGCTGGCCAACCCGCTGGTGGACCAGT |
| | | ACTTGTACCGCTTCGTGAGCACAAATAACACTGGCGGAGTCCAGTTCAACAA |
| | | GAACCTGGCCGGGAGATACGCCAACACCTACAAAAACTGGTTCCCGGGGCCC |
| | | ATGGGCCGAACCCAGGGCTGGAACCTGGGCTCCGGGGTCAACCGCGCCAGTG |
| | | TCAGCGCCTTCGCCACGACCAATAGGATGGAGCTCGAGGGCGCGAGTTACCA |
| | | GGTGCCCCCGCAGCCGAACGGCATGACCAACAACCTCCAGGGCAGCAACACC |
| | | TATGCCCTGGAGAACACTATGATCTTCAACAGCCAGCCGGCGAACCCGGGCA |
| | | CCACCGCCACGTACCTCGAGGGCAACATGCTCATCACCAGCGAGAGCGAGAC |
| | | GCAGCCGGTGAACCGCGTGGCGTACAACGTCGGCGGGCAGATGGCCACCAAC |
| | | AACCAGAGCTCCACCACTGCCCCCGCGACCGGCACGTACAACCTCCAGGAAA |
| | | TCGTGCCCGGCAGCGTGTGGATGGAGAGGGACGTGTACCTCCAAGGACCCAT |
| | | CTGGGCCAAGATCCCAGAGACGGGGGCGCACTTTCACCCCTCTCCGGCCATG |
| | | GGCGGATTCGGACTCAAACACCCACCGCCCATGATGCTCATCAAGAACACGC |
| | | CTGTGCCCGGAAATATCACCAGCTTCTCGGACGTGCCCGTCAGCAGCTTCAT |
| | | CACCCAGTACAGCACCGGGCAGGTCACCGTGGAGATGGAGTGGGAGCTCAAG |
| | | AAGGAAAACTCCAAGAGGTGGAACCCAGAGATCCAGTACACAAACAACTACA |
| | | ACGACCCCCAGTTTGTGGACTTTGCCCCGGACAGCACCGGGGAATACAGAAC |
| | | CACCAGACCTATCGGAACCCGATACCTTACCCGACCCCTTTAA |

In some embodiments, a packaging plasmid comprises a polyadenylation sequence (pA). In some embodiments, a pA sequence is downstream of a coding region. In some embodiments, a pA sequence is 3′ of a coding region. In some embodiments, a packaging plasmid comprises a p5 promoter sequence downstream of pA sequence. In some embodiments, a packaging plasmid comprises a p5 promoter sequence 3′ of pA sequence.

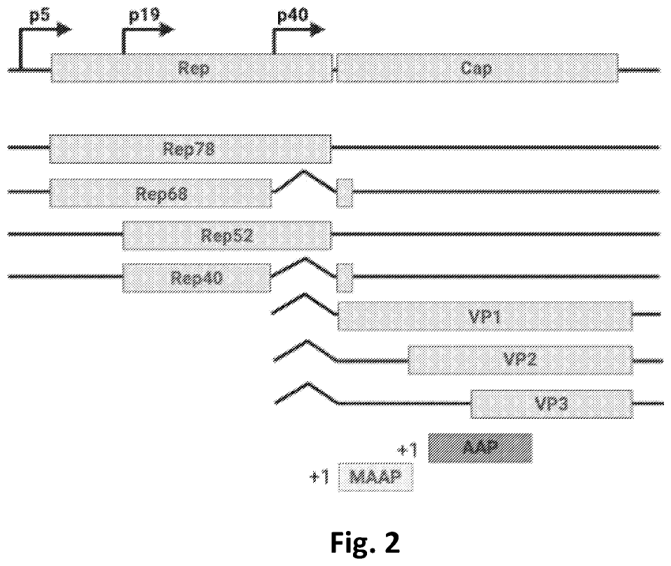

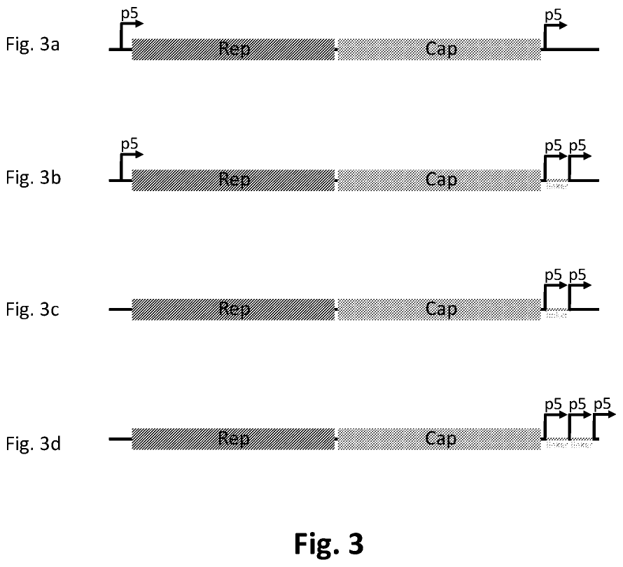

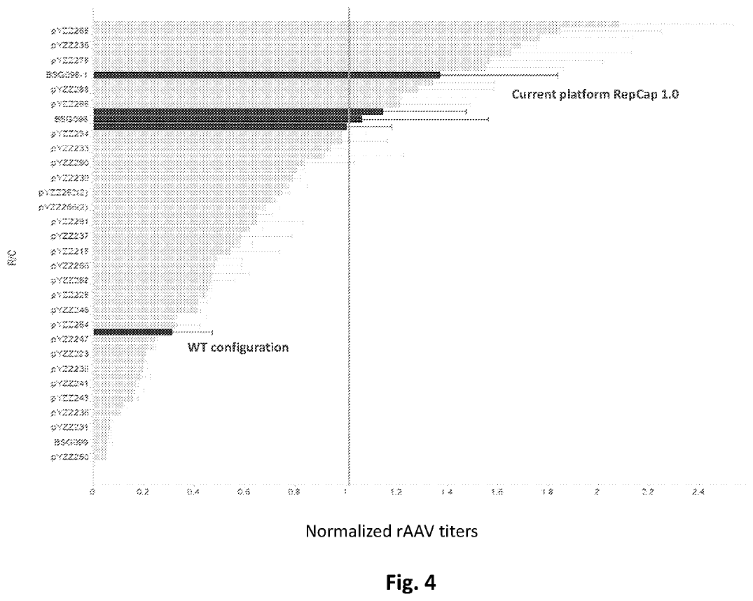

In some embodiments, a packaging plasmid comprises promoters. In some embodiments, a packaging plasmid comprises, among others, a p40 promoter regulating transcription from the Cap gene. In some embodiments, a packaging plasmid comprises, promoters (e.g., p5 and p19) regulating transcription from the Rep gene (FIG. 2).

### p5 Promoter Sequences

In some embodiments, a packaging plasmid comprises one or more p5 promoter sequences. In some embodiments, a packaging plasmid comprises a p5 promoter sequence upstream of a coding region. In some embodiments, a packaging plasmid comprises a p5 promoter sequence 5′ of a coding region. In some embodiments, a packaging plasmid comprises one or more p5 promoter sequence(s) downstream of a coding region. In some embodiments, a packaging plasmid comprises one or more p5 promoter sequence(s) 3′ of a coding region.

In some embodiments, a packaging plasmid comprises more than one p5 promoter sequences. In some embodiments, a packaging plasmid comprises more than one p5 promoter sequence downstream of a coding region. In some embodiments, a packaging plasmid comprises more than one p5 promoter sequence 3′ of a coding region.

In some embodiments, a packaging plasmid comprises one, two, or three p5 promoter sequence(s). In some embodiments, a packaging plasmid comprises one, two, or three p5 promoter sequence(s) downstream of a coding region. In some embodiments, a packaging plasmid comprises one, two, or three p5 promoter sequence(s) 3′ of a coding region.

In some embodiments, a packaging plasmid comprises one p5 promoter sequence 3′ of a coding region. In some embodiments, a packaging plasmid comprises two p5 promoter sequences 3′ of a coding region. In some embodiments, a packaging plasmid comprises three p5 promoter sequences 3′ of a coding region. In some embodiments, a packaging plasmid comprises one p5 promoter sequence downstream of a coding region. In some embodiments, a packaging plasmid comprises two p5 promoter sequences downstream of a coding region (e.g., a first p5 promoter sequence and a second p5 promoter sequence). In some embodiments, a packaging plasmid comprises three p5 promoter sequences downstream of a coding region (e.g., a first p5 promoter sequence, a second p5 promoter sequence and a third p5 promoter sequence).

In some embodiments, a packaging plasmid comprises a p5 promoter sequence upstream of a coding region and one, two, or three p5 promoter sequence(s) downstream of a coding region. In some embodiments, a packaging plasmid comprises a p5 promoter sequence 5′ of a coding region and one, two, or three p5 promoter sequence(s) 3′ of a coding region.

In some embodiments, a packaging plasmid comprises a p5 promoter sequence upstream of a coding region and one p5 promoter sequence downstream of a coding region. In some embodiments, a packaging plasmid comprises a p5 promoter sequence 5′ of a coding region and one p5 promoter sequence 3′ of a coding region.

In some embodiments, a packaging plasmid comprises a p5 promoter sequence upstream of a coding region and two p5 promoter sequences downstream of a coding region. In some embodiments, a packaging plasmid comprises a p5 promoter sequence upstream of a coding region, a first p5 promoter sequence and a second p5 promoter sequence downstream of a coding region. In some embodiments, a packaging plasmid comprises a p5 promoter sequence 5′ of a coding region and two p5 promoter sequences 3′ of a coding region. In some embodiments, a packaging plasmid comprises a p5 promoter sequence 5′ of a coding region, a first p5 promoter sequence and a second p5 promoter sequence 3′ of a coding region.

In some embodiments, a p5 promoter sequence is or comprises a sequence 80%, 85%, 90%, 95%, or 99% identical to a wild-type p5 sequence (SEQ ID NO: 4). In some embodiments, a p5 promoter sequence is or comprises a wild-type p5 sequence (SEQ ID NO: 4). In some embodiments, a p5 promoter sequence is or comprises a variant p5 sequence. In some embodiments, a p5 promoter sequence is or comprises a variant p5 sequence 80%, 85%, 90%, 95%, or 99% identical to any one of SEQ ID NO: 5 to SEQ ID NO: 15. In some embodiments, a p5 promoter sequence is or comprises a variant p5 sequence selected from any one of SEQ ID NO: 5 to SEQ ID NO: 15. In some embodiments, a p5 promoter sequence is or comprises a sequence selected from the sequences listed in Table 2.

TABLE 2
Exemplary p5 sequences

| SEQ ID NO: | Name | Sequence |
|---|---|---|
| 4 | P5 | GAGGTCCTGTATTAGAGGTCACGTGAGTGTTTTGCGACATTTTGCGACA |
| | | CCATGTGGTCACGCTGGGTATTTAAGCCCGAGTGAGCACGCAGGGTCTC |
| | | CATTTTGAAGCGGGAGGTTTGAA |
| 5 | P5 variant 1 | AGGGTCTCCATTTTGAAGCGGGAGGTTTGAACGCGCAGCCGCC |
| 6 | P5 variant 2 | GCCCGAGTGAGCACGCAGGTTTGAACGCGCAGCCGCC |
| 7 | P5 variant 3 | GCCCGAGTGAGCACGCAGGGTCTCCATTTTGAAGCGGGAGGTTTGAACG |
| | | CGCAGCCGCC |
| 8 | P5 variant 4 | TATTTAAGCCCGAGTGAGCACGCAGGGTCTCCATTTTGAAGCGGGAGGT |
| | | TTGAACGCGCAGCCGCC |
| 9 | P5 variant 5 | GAGGTCCTGTATTAGATTTGCGACATTTTGCGACACCATGTGGTCACGC |
| | | TGGGTATTTAAGCCCGAGTGAGCACGCAGGGTCTCCATTTTGAAGCGGG |
| | | AGGTTTGAACGCGCAGCCGCC |
| 10 | P5 variant 6 | GAGGTCCTGTATTAGAGGTCACGTGAGTGTCATGTGGTCACGCTGGGTA |
| | | TTTAAGCCCGAGTGAGCACGCAGGGTCTCCATTTTGAAGCGGGAGGTTT |
| | | GAACGCGCAGCCGCC |
| 11 | P5 variant 7 | GAGGTCCTGTATTAGACATGTGGTCACGCTGGGTATTTAAGCCCGAGTG |
| | | AGCACGCAGGGTCTCCATTTTGAAGCGGGAGGTTTGAACGCGCAGCCGC |
| | | C |
| 12 | P5 variant 8 | GAGGTCCTGTATTAGATTTGCGACATTTTGCGACACCATGTGGTCACGC |
| | | TGGGGCCCGAGTGAGCACGCAGGGTCTCCATTTTGAAGCGGGAGGTTTG |
| | | AACGCGCAGCCGCC |
| 13 | P5 variant 9 | GAGGTCCTGTATTAGAGGTCACGTGAGTGTCATGTGGTCACGCTGGGGC |
| | | CCGAGTGAGCACGCAGGGTCTCCATTTTGAAGCGGGAGGTTTGAACGCG |
| | | CAGCCGCC |
| 14 | P5 variant 10 | GAGGTCCTGTATTAGACATGTGGTCACGCTGGGGCCCGAGTGAGCACGC |
| | | AGGGTCTCCATTTTGAAGCGGGAGGTTTGAACGCGCAGCCGCC |
| 15 | P5 variant 11 | GAGGTCCTGTATTAGAGCCCGAGTGAGCACGCAGGGTCTCCATTTTGAA |
| | | GCGGGAGGTTTGAACGCGCAGCCGCC |

### Linker Sequences

In some embodiments, a packaging plasmid described herein comprises one or more linker sequence(s). In some embodiments, a packaging plasmid comprises a linker sequence between one or more p5 promoter sequence(s). In some embodiments, a packaging plasmid comprises a linker sequence between one or more p5 promoter sequence(s) downstream of a coding region. In some embodiments, a packaging plasmid comprises a linker sequence between one or more p5 promoter sequence(s) 5′ of a coding region.

In some embodiments, a packaging plasmid comprises one or more linker sequences. In some embodiments, a packaging plasmid comprises one or more linker sequences between any two p5 promoter sequences. In some embodiments, a packaging plasmid comprises a linker sequence between two p5 promoter sequences. In some embodiments, a packaging plasmid comprising more than two p5 promoter sequences comprises a linker sequence between each pair of (e.g., set of two) p5 sequences. In some embodiments, a packaging plasmid comprising a first p5 promoter sequence and a second p5 promoter sequence comprises a linker sequence between the first p5 sequence and the second p5 promoter sequence. In some embodiments, a packaging plasmid comprising three p5 sequences comprises two linker sequences with one linker sequence between each pair of (e.g., set of two) p5 promoter sequences. In some embodiments, a packaging plasmid comprising a first p5 promoter sequence, a second p5 promoter sequence and a third p5 promoter sequence comprises a linker sequence between the first p5 promoter sequence and the second p5 promoter sequence and a linker sequence between the second p5 promoter sequence and the third p5 promoter sequence.

In some embodiments, a packaging plasmid comprises one or more linker sequences downstream of a coding region. In some embodiments, a packaging plasmid comprises a linker sequence between any two p5 sequences downstream of a coding region. In some embodiments, a packaging plasmid comprises a linker sequence between two p5 promoter sequences downstream of a coding region. In some embodiments, a packaging plasmid comprising three p5 sequences downstream of a coding region comprises two linkers with one linker between each pair of (e.g., set of two) p5 sequences.

In some embodiments, a packaging plasmid comprises one or more linker sequences 5′ of a coding region. In some embodiments, a packaging plasmid comprises a linker sequence between any two p5 promoter sequences 5′ of a coding region. In some embodiments, a packaging plasmid comprises a linker sequence between two p5 promoter sequences 5′ of a coding region. In some embodiments, a packaging plasmid comprising three p5 promoter sequences 5′ of a coding region comprises two linkers with one linker between each pair of (e.g., set of two) p5 sequences.

In some embodiments, a linker sequence is between 1-500 bp; 1-500 bp, 1-450 bp, 1-400 bp, 1-350 bp, 1-300 bp, 1-250 bp, 1-200 bp, 50-500 bp, 50-450 bp, 50-400 bp, 50-350 bp, 50-300 bp, 50-250 bp, 50-200 bp, 100-500 bp, 100-400 bp, 100 bp-300 bp, and 100-200 bp in length. In some embodiments, a linker sequence is 80%, 85%, 90%, 95%, or 99% identical to a sequence selected from Table 3. In some embodiments, a linker sequence is selected from the sequences of Table 3.

TABLE 3
Exemplary linker sequences

| SEQ ID NO: | Name | Sequence |
|---|---|---|
| 16 | Linker 1 | CGCGCAGCCGCCAAGCCGAATTCTGCAGATCCGGTTAATTCGTTTCAGTT |
| | | GAACTTTGGTCTCCTGTCCTTCTTATCTTATCGGTTACCATAGAAACTGG |
| | | TTACTTATTAACTGCTTGGTGCGCTTCGCGATAAAAGACTTACGTCATCG |
| | | GGTTACCCCTAGTGATGGAGCGGCCGCTTTCAGTTGAACTTTGGTCTCTG |
| | | CGTATTTCTTTCTTATCTAGTTTCCATGCTCTA |
| 17 | Linker 2 | CGCGCAGCCGCCAAGCCGTTTCAGTTGAACTTTGGTCTCTGCGTATTTCT |
| | | TTCTTATCTAGTTTCCATG |
| 18 | Linker 3 | GTCTTACCCCCAATAAGGGGATTTGCTCTATTTAATTAGGAATAAGGTCG |
| | | ATTACTGATAGAACAAATCCAGGCTACTGTGTTTAGTAATCAGATTTGTT |
| 19 | Linker 4 | GTCTTACCCCCAATAAGGGGATTTGCTCTATTTAATTAGGAATAAGGTCG |
| | | ATTACTGATAGAACAAATCCAGGCTACTGTGTTTAGTAATCAGATTTGTT |
| | | CGTGACCGATATGCACGGGCAAAACGGCAGGAGGTTGTTAGCGCGACCTC |
| | | CTGCCACCCGCTTTCACGAAGGTCATGTGTAAAAGGCCGCAGCGTAACTA |
| 20 | Linker 5 | GTCTTACCCCCAATAAGGGGATTTGCTCTATTTAATTAGGAATAAGGTCG |
| | | ATTACTGATAGAACAAATCCAGGCTACTGTGTTTAGTAATCAGATTTGTT |
| | | CGTGACCGATATGCACGGGCAAAACGGCAGGAGGTTGTTAGCGCGACCTC |
| | | CTGCCACCCGCTTTCACGAAGGTCATGTGTAAAAGGCCGCAGCGTAACTA |
| | | TTACTAATGAATTCAGGACAGACAGTGGCTACGGCTCAGTTTGGGTTGTG |
| | | CTGTTGCTGGGCGGCGATGACGCCTGTACGCATTTGGTGATCCGGTTCTG |
| 21 | Linker 6 | CATACAGACGAAACTAGTTGGAGATTATCGAGCATTCTATCACGTCGACG |
| | | ACCACTAGTGAGTTACTAGAGCCGAGGGGTAACGTTGATGCTCCTAAAAA |
| 22 | Linker 7 | CATACAGACGAAACTAGTTGGAGATTATCGAGCATTCTATCACGTCGACG |
| | | ACCACTAGTGAGTTACTAGAGCCGAGGGGTAACGTTGATGCTCCTAAAAA |
| | | TCTCTCGGTCGATGCAAGCGATTACACTCCTGTCACATCATAATCGTTTG |
| | | CTATTCAGGGCTTGATCAACACTGGATTGCTTTTCACTTAAAGTATTATG |
| 23 | Linker 8 | CATACAGACGAAACTAGTTGGAGATTATCGAGCATTCTATCACGTCGACG |
| | | ACCACTAGTGAGTTACTAGAGCCGAGGGGTAACGTTGATGCTCCTAAAAA |
| | | TCTCTCGGTCGATGCAAGCGATTACACTCCTGTCACATCATAATCGTTTG |
| | | CTATTCAGGGCTTGATCAACACTGGATTGCTTTTCACTTAAAGTATTATG |
| | | CACGACAGGGTACGTGTACCATATAAACCTGTTATAACTTACCTCAGACT |
| | | AGTTGGAAATGTGGCTAGATCTTAGCTTACGTTACTAAAGGGTCCACGTT |

### Exemplary AAV Construct Sequences

In some embodiments, packaging plasmid described herein has a nucleotide sequence and configuration as provided in Table 4. Nucleotide sequences are colored according to the following scheme; p5 promoter, Rep2 (ATG), AltRep2 (ACG), Cap9, Cap5, or Cap2, AAV2polyA, linkers. Configuration naming is as follows: p5 stands for a p5 promoter sequence (e.g., SEQ ID NO: 4). P5v stands for variant p5 promoter sequence (e.g., SEQ ID NO: 5-15). L stands for linker sequence with the number following this letter indicating the number linker sequence and associated nucleotide sequence as listed in SEQ ID NO: 16-23. Rep2 stands for the replication 2 nucleotide sequence as listed in SEQ ID NO: 1. AltRep2 stands for the alternative replication 2 nucleotide sequence as listed in SEQ ID NO: 2. Cap9 stands for Capsid 9 nucleotide sequence as listed in SEQ ID NO: 3. Cap2 stands for Capsid 2 nucleotide sequence as listed in SEQ ID NO: 50. Cap5 stands for Capsid 5 nucleotide sequence as listed in SEQ ID NO: 51.

TABLE 4
Exemplary plasmid sequences and configurations

| SEQ | | | |
|---|---|---|---|
| ID | | | |
| NO: | Plasmid | Configuration | Sequence |
| 24 | pYZZ233 | p5-Rep2.Cap9- | TCGCGCGTTTCGGTGATGACGGTGAAAACCTCTGA |
| | | p5.L1.p5 | CACATGCAGCTCCCGGAGACGGTCACAGCTTGTCT |
| | | | GTAAGCGGATGCCGGGAGCAGACAAGCCCGTCAG |
| | | | GGCGCGTCAGCGGGTGTTGGCGGGTGTCGGGGCTG |
| | | | GCTTAACTATGCGGCATCAGAGCAGATTGTACTGA |
| | | | GAGTGCACCATATGCGGTGTGAAATACCGCACAGA |
| | | | TGCGTAAGGAGAAAATACCGCATCAGGCGCCATTC |
| | | | GCCATTCAGGCTGCGCAACTGTTGGGAAGGGCGAT |
| | | | CGGTGCGGGCCTCTTCGCTATTACGCCAGCTGGCG |
| | | | AAAGGGGGATGTGCTGCAAGGCGATTAAGTTGGGT |
| | | | AACGCCAGGGTTTTCCCAGTCACGACGTTGTAAAA |
| | | | CGACGGCCAGTGAATTGACGCGTATTGGGATGAGG |
| | | | TCCTGTATTAGAGGTCACGTGAGTGTTTTGCGA |
| | | | CATTTTGCGACACCATGTGGTCACGCTGGGTAT |
| | | | TTAAGCCCGAGTGAGCACGCAGGGTCTCCATTT |
| | | | TGAAGCGGGAGGTTTGAACGCGCAGCCGCCATGC |
| | | | CGGGGTTTTACGAGATTGTGATTAAGGTCCCCAGCGA |
| | | | CCTTGACGAGCATCTGCCCGGCATTTCTGACAGCTTTG |
| | | | TGAACTGGGTGGCCGAGAAGGAATGGGAGTTGCCGC |
| | | | CAGATTCTGACATGGATCTGAATCTGATTGAGCAGGCA |
| | | | CCCCTGACCGTGGCCGAGAAGCTGCAGCGCGACTTTC |
| | | | TGACGGAATGGCGCCGTGTGAGTAAGGCCCCGGAGG |
| | | | CTCTTTTCTTTGTGCAATTTGAGAAGGGAGAGAGCTAC |
| | | | TTCCACATGCACGTGCTCGTGGAAACCACCGGGGTGA |
| | | | AATCCATGGTTTTGGGACGTTTCCTGAGTCAGATTCGC |
| | | | GAAAAACTGATTCAGAGAATTTACCGCGGGATCGAGC |
| | | | CGACTTTGCCAAACTGGTTCGCGGTCACAAAGACCAG |
| | | | AAATGGCGCCGGAGGCGGGAACAAGGTGGTGGATGA |
| | | | GTGCTACATCCCCAATTACTTGCTCCCCAAAACCCAGC |
| | | | CTGAGCTCCAGTGGGCGTGGACTAATATGGAACAGTA |
| | | | TTTAAGCGCCTGTTTGAATCTCACGGAGCGTAAACGGT |
| | | | TGGTGGCGCAGCATCTGACGCACGTGTCGCAGACGCA |
| | | | GGAGCAGAACAAAGAGAATCAGAATCCCAATTCTGATG |
| | | | CGCCGGTGATCAGATCAAAAACTTCAGCCAGGTACAT |
| | | | GGAGCTGGTCGGGTGGCTCGTGGACAAGGGGATTAC |
| | | | CTCGGAGAAGCAGTGGATCCAGGAGGACCAGGCCTC |
| | | | ATACATCTCCTTCAATGCGGCCTCCAACTCGCGGTCCC |
| | | | AAATCAAGGCTGCCTTGGACAATGCGGGAAAGATTATG |
| | | | AGCCTGACTAAAACCGCCCCCGACTACCTGGTGGGCC |
| | | | AGCAGCCCGTGGAGGACATTTCCAGCAATCGGATTTAT |
| | | | AAAATTTTGGAACTAAACGGGTACGATCCCCAATATGC |
| | | | GGCTTCCGTCTTTCTGGGATGGGCCACGAAAAAGTTC |
| | | | GGCAAGAGGAACACCATCTGGCTGTTTGGGCCTGCAA |
| | | | CTACCGGGAAGACCAACATCGCGGAGGCCATAGCCCA |
| | | | CACTGTGCCCTTCTACGGGTGCGTAAACTGGACCAAT |
| | | | GAGAACTTTCCCTTCAACGACTGTGTCGACAAGATGGT |
| | | | GATCTGGTGGGAGGAGGGGAAGATGACCGCCAAGGT |
| | | | CGTGGAGTCGGCCAAAGCCATTCTCGGAGGAAGCAAG |
| | | | GTGCGCGTGGACCAGAAATGCAAGTCCTCGGCCCAGA |
| | | | TAGACCCGACTCCCGTGATCGTCACCTCCAACACCAA |
| | | | CATGTGCGCCGTGATTGACGGGAACTCAACGACCTTC |
| | | | GAACACCAGCAGCCGTTGCAAGACCGGATGTTCAAAT |
| | | | TTGAACTCACCCGCCGTCTGGATCATGACTTTGGGAAG |
| | | | GTCACCAAGCAGGAAGTCAAAGACTTTTTCCGGTGGG |
| | | | CAAAGGATCACGTGGTTGAGGTGGAGCATGAATTCTA |
| | | | CGTCAAAAAGGGTGGAGCCAAGAAAAGACCCGCCCCC |
| | | | AGTGACGCAGATATAAGTGAGCCCAAACGGGTGCGCG |
| | | | AGTCAGTTGCGCAGCCATCGACGTCAGACGCGGAAGC |
| | | | TTCGATCAACTACGCAGACAGGTACCAAAACAAATGTT |
| | | | CTCGTCACGTGGGCATGAATCTGATGCTGTTTCCCTGC |
| | | | AGACAATGCGAGAGAATGAATCAGAATTCAAATATCTG |
| | | | CTTCACTCACGGACAGAAAGACTGTTTAGAGTGCTTTC |
| | | | CCGTGTCAGAATCTCAACCCGTTTCTGTCGTCAAAAAG |
| | | | GCGTATCAGAAACTGTGCTACATTCATCATATCATGGG |
| | | | AAAGGTGCCAGACGCTTGCACTGCCTGCGATCTGGTC |
| | | | AATGTGGATTTGGATGACTGCATCTTTGAACAATAAAT |
| | | | GATTTAAATCAGGTATGGCTGCCGATGGTTATCTTC |
| | | | CAGATTGGCTCGAGGACAACCTTAGTGAAGGAATT |
| | | | CGCGAGTGGTGGGCTTTGAAACCTGGAGCCCCTCA |
| | | | ACCCAAGGCAAATCAACAACATCAAGACAACGCTC |
| | | | GAGGTCTTGTGCTTCCGGGTTACAAATACCTTGGAC |
| | | | CCGGCAACGGACTCGACAAGGGGGAGCCGGTCAA |
| | | | CGCAGCAGACGCGGCGGCCCTCGAGCACGACAAG |
| | | | GCCTACGACCAGCAGCTCAAGGCCGGAGACAACCC |
| | | | GTACCTCAAGTACAACCACGCCGACGCCGAGTTCC |
| | | | AGGAGCGGCTCAAAGAAGATACGTCTTTTGGGGGC |
| | | | AACCTCGGGCGAGCAGTCTTCCAGGCCAAAAAGAG |
| | | | GCTTCTTGAACCTCTTGGTCTGGTTGAGGAAGCGGC |
| | | | TAAGACGGCTCCTGGAAAGAAGAGGCCTGTAGAGC |
| | | | AGTCTCCTCAGGAACCGGACTCCTCCGCGGGTATT |
| | | | GGCAAATCGGGTGCACAGCCCGCTAAAAAGAGACT |
| | | | CAATTTCGGTCAGACTGGCGACACAGAGTCAGTCC |
| | | | CAGACCCTCAACCAATCGGAGAACCTCCCGCAGCC |
| | | | CCCTCAGGTGTGGGATCTCTTACAATGGCTTCAGGT |
| | | | GGTGGCGCACCAGTGGCAGACAATAACGAAGGTG |
| | | | CCGATGGAGTGGGTAGTTCCTCGGGAAATTGGCAT |
| | | | TGCGATTCCCAATGGCTGGGGGACAGAGTCATCAC |
| | | | CACCAGCACCCGAACCTGGGCCCTGCCCACCTACA |
| | | | ACAATCACCTCTACAAGCAAATCTCCAACAGCACA |
| | | | TCTGGAGGATCTTCAAATGACAACGCCTACTTCGG |
| | | | CTACAGCACCCCCTGGGGGTATTTTGACTTCAACA |
| | | | GATTCCACTGCCACTTCTCACCACGTGACTGGCAGC |
| | | | GACTCATCAACAACAACTGGGGATTCCGGCCTAAG |
| | | | CGACTCAACTTCAAGCTCTTCAACATTCAGGTCAA |
| | | | AGAGGTTACGGACAACAATGGAGTCAAGACCATCG |
| | | | CCAATAACCTTACCAGCACGGTCCAGGTCTTCACG |
| | | | GACTCAGACTATCAGCTCCCGTACGTGCTCGGGTC |
| | | | GGCTCACGAGGGCTGCCTCCCGCCGTTCCCAGCGG |
| | | | ACGTTTTCATGATTCCTCAGTACGGGTATCTGACGC |
| | | | TTAATGATGGAAGCCAGGCCGTGGGTCGTTCGTCC |
| | | | TTTTACTGCCTGGAATATTTCCCGTCGCAAATGCTA |
| | | | AGAACGGGTAACAACTTCCAGTTCAGCTACGAGTT |
| | | | TGAGAACGTACCTTTCCATAGCAGCTACGCTCACA |
| | | | GCCAAAGCCTGGACCGACTAATGAATCCACTCATC |
| | | | GACCAATACTTGTACTATCTCTCAAAGACTATTAAC |
| | | | TGTGGCCGGACCCAGCAACATGGCTGTCCAGGGAA |
| | | | GAAACTACATACCTGGACCCAGCTACCGACAACAA |
| | | | CGTGTCTCAACCACTGTGACTCAAAACAACAACAG |
| | | | CGAATTTGCTTGGCCTGGAGCTTCTTCTTGGGCTCT |
| | | | CAATGGACGTAATAGCTTGATGAATCCTGGACCTG |
| | | | CTATGGCCAGCCACAAAGAAGGAGAGGACCGTTTC |
| | | | TTTCCTTTGTCTGGATCTTTAATTTTTGGCAAACAA |
| | | | GGAACTGGAAGAGACAACGTGGATGCGGACAAAG |
| | | | TCATGATAACCAACGAAGAAGAAATTAAAACTACT |
| | | | AACCCGGTAGCAACGGAGTCCTATGGACAAGTGGC |
| | | | CACAAACCACCAGAGTGCCCAAGCACAGGCGCAG |
| | | | ACCGGCTGGGTTCAAAACCAAGGAATACTTCCGGG |
| | | | TATGGTTTGGCAGGACAGAGATGTGTACCTGCAAG |
| | | | GACCCATTTGGGCCAAAATTCCTCACACGGACGGC |
| | | | AACTTTCACCCTTCTCCGCTGATGGGAGGGTTTGGA |
| | | | ATGAAGCACCCGCCTCCTCAGATCCTCATCAAAAA |
| | | | CACACCTGTACCTGCGGATCCTCCAACGGCCTTCA |
| | | | ACAAGGACAAGCTGAACTCTTTCATCACCCAGTAT |
| | | | TCTACTGGCCAAGTCAGCGTGGAGATCGAGTGGGA |
| | | | GCTGCAGAAGGAAAACAGCAAGCGCTGGAACCCG |
| | | | GAGATCCAGTACACTTCCAACTATTACAAGTCTAA |
| | | | TAATGTTGAATTTGCTGTTAATACTGAAGGTGTATA |
| | | | TAGTGAACCCCGCCCCATTGGCACCAGATACCTGA |
| | | | CTCGTAATCTGTAATTGCTTGTTAATCAATAAACC |
| | | | GTTTAATTCGTTTCAGTTGAACTTTGGTCTCTGCG |
| | | | AAGGGCGAATTCGTTTAAACCTGCAGGACTAGAGG |
| | | | TCCTGTATTAGAGGTCACGTGAGTGTTTTGCGA |
| | | | CATTTTGCGACACCATGTGGTCACGCTGGGTAT |
| | | | TTAAGCCCGAGTGAGCACGCAGGGTCTCCATTT |
| | | | TGAAGCGGGAGGTTTGAACGCGCAGCCGCCAAGC |
| | | | CGAATTCTGCAGATCCGGTTAATTCGTTTCAGTTGAAC |
| | | | TTTGGTCTCCTGTCCTTCTTATCTTATCGGTTACCATAG |
| | | | AAACTGGTTACTTATTAACTGCTTGGTGCGCTTCGCGA |
| | | | TAAAAGACTTACGTCATCGGGTTACCCCTAGTGATGGA |
| | | | GCGGCCGCTTTCAGTTGAACTTTGGTCTCTGCGTATTT |
| | | | CTTTCTTATCTAGTTTCCATGCTCTAGAGGTCCTGTA |
| | | | TTAGAGGTCACGTGAGTGTTTTGCGACATTTTG |
| | | | CGACACCATGTGGTCACGCTGGGTATTTAAGCC |
| | | | CGAGTGAGCACGCAGGGTCTCCATTTTGAAGCG |
| | | | GGAGGTTTGAACGCGCAGCCGCCAAGCCGAATTC |
| | | | TGCAGAATCCCAATGGCGCGCCGAGCTTGGCTCGA |
| | | | GCATGGTCATAGCTGTTTCCTGTGTGAAATTGTTAT |
| | | | CCGCTCACAATTCCACACAACATACGAGCCGGAAG |
| | | | CATAAAGTGTAAAGCCTGGGGTGCCTAATGAGTGA |
| | | | GCTAACTCACATTAATTGCGTTGCGCTCACTGCCCG |
| | | | CTTTCCAGTCGGGAAACCTGTCGTGCCAGCTGCATT |
| | | | AATGAATCGGCCAACGCGCGGGGAGAGGCGGTTTG |
| | | | CGTATTGGGCGCTCTTCCGCTTCCTCGCTCACTGAC |
| | | | TCGCTGCGCTCGGTCGTTCGGCTGCGGCGAGCGGT |
| | | | ATCAGCTCACTCAAAGGCGGTAATACGGTTATCCA |
| | | | CAGAATCAGGGGATAACGCAGGAAAGAACATGTG |
| | | | AGCAAAAGGCCAGCAAAAGGCCAGGAACCGTAAA |
| | | | AAGGCCGCGTTGCTGGCGTTTTTCCATAGGCTCCGC |
| | | | CCCCCTGACGAGCATCACAAAAATCGACGCTCAAG |
| | | | TCAGAGGTGGCGAAACCCGACAGGACTATAAAGAT |
| | | | ACCAGGCGTTTCCCCCTGGAAGCTCCCTCGTGCGCT |
| | | | CTCCTGTTCCGACCCTGCCGCTTACCGGATACCTGT |
| | | | CCGCCTTTCTCCCTTCGGGAAGCGTGGCGCTTTCTC |
| | | | ATAGCTCACGCTGTAGGTATCTCAGTTCGGTGTAG |
| | | | GTCGTTCGCTCCAAGCTGGGCTGTGTGCACGAACC |
| | | | CCCCGTTCAGCCCGACCGCTGCGCCTTATCCGGTAA |
| | | | CTATCGTCTTGAGTCCAACCCGGTAAGACACGACT |
| | | | TATCGCCACTGGCAGCAGCCACTGGTAACAGGATT |
| | | | AGCAGAGCGAGGTATGTAGGCGGTGCTACAGAGTT |
| | | | CTTGAAGTGGTGGCCTAACTACGGCTACACTAGAA |
| | | | GAACAGTATTTGGTATCTGCGCTCTGCTGAAGCCA |
| | | | GTTACCTTCGGAAAAAGAGTTGGTAGCTCTTGATC |
| | | | CGGCAAACAAACCACCGCTGGTAGCGGTGGTTTTT |
| | | | TTGTTTGCAAGCAGCAGATTACGCGCAGAAAAAAA |
| | | | GGATCTCAAGAAGATCCTTTGATCTTTTCTACGGGG |
| | | | TCTGACGCTCAGTGGAACGAAAACTCACGTTAAGG |
| | | | GATTTTGGTCATGAGATTATCAAAAAGGATCTTCA |
| | | | CCTAGATCCTTTTAAATTAAAAATGAAGTTTTAAAT |
| | | | CAATCTAAAGTATATATGAGTAAACTTGGTCTGAC |
| | | | AGTTAGAAAAACTCATCGAGCATCAAATGAAACTG |
| | | | CAATTTATTCATATCAGGATTATCAATACCATATTT |
| | | | TTGAAAAAGCCGTTTCTGTAATGAAGGAGAAAACT |
| | | | CACCGAGGCAGTTCCATAGGATGGCAAGATCCTGG |
| | | | TATCGGTCTGCGATTCCGACTCGTCCAACATCAATA |
| | | | CAACCTATTAATTTCCCCTCGTCAAAAATAAGGTTA |
| | | | TCAAGTGAGAAATCACCATGAGTGACGACTGAATC |
| | | | CGGTGAGAATGGCAAAAGTTTATGCATTTCTTTCCA |
| | | | GACTTGTTCAACAGGCCAGCCATTACGCTCGTCATC |
| | | | AAAATCACTCGCATCAACCAAACCGTTATTCATTC |
| | | | GTGATTGCGCCTGAGCGAGACGAAATACGCGATCG |
| | | | CTGTTAAAAGGACAATTACAAACAGGAATCGAATG |
| | | | CAACCGGCGCAGGAACACTGCCAGCGCATCAACAA |
| | | | TATTTTCACCTGAATCAGGATATTCTTCTAATACCT |
| | | | GGAATGCTGTTTTCCCAGGGATCGCAGTGGTGAGT |
| | | | AACCATGCATCATCAGGAGTACGGATAAAATGCTT |
| | | | GATGGTCGGAAGAGGCATAAATTCCGTCAGCCAGT |
| | | | TTAGTCTGACCATCTCATCTGTAACATCATTGGCAA |
| | | | CGCTACCTTTGCCATGTTTCAGAAACAACTCTGGCG |
| | | | CATCGGGCTTCCCATACAATCGATAGATTGTCGCA |
| | | | CCTGATTGCCCGACATTATCGCGAGCCCATTTATAC |
| | | | CCATATAAATCAGCATCCATGTTGGAATTTAATCGC |
| | | | GGCCTAGAGCAAGACGTTTCCCGTTGAATATGGCT |
| | | | CATACTCTTCCTTTTTCAATATTATTGAAGCATTTAT |
| | | | CAGGGTTATTGTCTCATGAGCGGATACATATTTGA |
| | | | ATGTATTTAGAAAAATAAACAAATAGGGGTTCCGC |
| | | | GCACATTTCCCCGAAAAGTGCCACCTGACGTCTAA |
| | | | GAAACCATTATTATCATGACATTAACCTATAAAAA |
| | | | TAGGCGTATCACGAGGCCCTTTCGTC |
| 25 | pYZZ234 | p5- | TCGCGCGTTTCGGTGATGACGGTGAAAACCTCTGA |
| | | AltRep2.Cap9- | CACATGCAGCTCCCGGAGACGGTCACAGCTTGTCT |
| | | p5.L1.p5 | GTAAGCGGATGCCGGGAGCAGACAAGCCCGTCAG |
| | | | GGCGCGTCAGCGGGTGTTGGCGGGTGTCGGGGCTG |
| | | | GCTTAACTATGCGGCATCAGAGCAGATTGTACTGA |
| | | | GAGTGCACCATATGCGGTGTGAAATACCGCACAGA |
| | | | TGCGTAAGGAGAAAATACCGCATCAGGCGCCATTC |
| | | | GCCATTCAGGCTGCGCAACTGTTGGGAAGGGCGAT |
| | | | CGGTGCGGGCCTCTTCGCTATTACGCCAGCTGGCG |
| | | | AAAGGGGGATGTGCTGCAAGGCGATTAAGTTGGGT |
| | | | AACGCCAGGGTTTTCCCAGTCACGACGTTGTAAAA |
| | | | CGACGGCCAGTGAATTGACGCGTATTGGGATGAGG |
| | | | TCCTGTATTAGAGGTCACGTGAGTGTTTTGCGA |
| | | | CATTTTGCGACACCATGTGGTCACGCTGGGTAT |
| | | | TTAAGCCCGAGTGAGCACGCAGGGTCTCCATTT |
| | | | TGAAGCGGGAGGTTTGAACGCGCAGCCGCCACGC |
| | | | CGGGGTTTTACGAGATTGTGATTAAGGTCCCCAGCGA |
| | | | CCTTGACGAGCATCTGCCCGGCATTTCTGACAGCTTTG |
| | | | TGAACTGGGTGGCCGAGAAGGAATGGGAGTTGCCGC |
| | | | CAGATTCTGACATGGATCTGAATCTGATTGAGCAGGCA |
| | | | CCCCTGACCGTGGCCGAGAAGCTGCAGCGCGACTTTC |
| | | | TGACGGAATGGCGCCGTGTGAGTAAGGCCCCGGAGG |
| | | | CTCTTTTCTTTGTGCAATTTGAGAAGGGAGAGAGCTAC |
| | | | TTCCACATGCACGTGCTCGTGGAAACCACCGGGGTGA |
| | | | AATCCATGGTTTTGGGACGTTTCCTGAGTCAGATTCGC |
| | | | GAAAAACTGATTCAGAGAATTTACCGCGGGATCGAGC |
| | | | CGACTTTGCCAAACTGGTTCGCGGTCACAAAGACCAG |
| | | | AAATGGCGCCGGAGGCGGGAACAAGGTGGTGGATGA |
| | | | GTGCTACATCCCCAATTACTTGCTCCCCAAAACCCAGC |
| | | | CTGAGCTCCAGTGGGCGTGGACTAATATGGAACAGTA |
| | | | TTTAAGCGCCTGTTTGAATCTCACGGAGCGTAAACGGT |
| | | | TGGTGGCGCAGCATCTGACGCACGTGTCGCAGACGCA |
| | | | GGAGCAGAACAAAGAGAATCAGAATCCCAATTCTGATG |
| | | | CGCCGGTGATCAGATCAAAAACTTCAGCCAGGTACAT |
| | | | GGAGCTGGTCGGGTGGCTCGTGGACAAGGGGATTAC |
| | | | CTCGGAGAAGCAGTGGATCCAGGAGGACCAGGCCTC |
| | | | ATACATCTCCTTCAATGCGGCCTCCAACTCGCGGTCCC |
| | | | AAATCAAGGCTGCCTTGGACAATGCGGGAAAGATTATG |
| | | | AGCCTGACTAAAACCGCCCCCGACTACCTGGTGGGCC |
| | | | AGCAGCCCGTGGAGGACATTTCCAGCAATCGGATTTAT |
| | | | AAAATTTTGGAACTAAACGGGTACGATCCCCAATATGC |
| | | | GGCTTCCGTCTTTCTGGGATGGGCCACGAAAAAGTTC |
| | | | GGCAAGAGGAACACCATCTGGCTGTTTGGGCCTGCAA |
| | | | CTACCGGGAAGACCAACATCGCGGAGGCCATAGCCCA |
| | | | CACTGTGCCCTTCTACGGGTGCGTAAACTGGACCAAT |
| | | | GAGAACTTTCCCTTCAACGACTGTGTCGACAAGATGGT |
| | | | GATCTGGTGGGAGGAGGGGAAGATGACCGCCAAGGT |
| | | | CGTGGAGTCGGCCAAAGCCATTCTCGGAGGAAGCAAG |
| | | | GTGCGCGTGGACCAGAAATGCAAGTCCTCGGCCCAGA |
| | | | TAGACCCGACTCCCGTGATCGTCACCTCCAACACCAA |
| | | | CATGTGCGCCGTGATTGACGGGAACTCAACGACCTTC |
| | | | GAACACCAGCAGCCGTTGCAAGACCGGATGTTCAAAT |
| | | | TTGAACTCACCCGCCGTCTGGATCATGACTTTGGGAAG |
| | | | GTCACCAAGCAGGAAGTCAAAGACTTTTTCCGGTGGG |
| | | | CAAAGGATCACGTGGTTGAGGTGGAGCATGAATTCTA |
| | | | CGTCAAAAAGGGTGGAGCCAAGAAAAGACCCGCCCCC |
| | | | AGTGACGCAGATATAAGTGAGCCCAAACGGGTGCGCG |
| | | | AGTCAGTTGCGCAGCCATCGACGTCAGACGCGGAAGC |
| | | | TTCGATCAACTACGCAGACAGGTACCAAAACAAATGTT |
| | | | CTCGTCACGTGGGCATGAATCTGATGCTGTTTCCCTGC |
| | | | AGACAATGCGAGAGAATGAATCAGAATTCAAATATCTG |
| | | | CTTCACTCACGGACAGAAAGACTGTTTAGAGTGCTTTC |
| | | | CCGTGTCAGAATCTCAACCCGTTTCTGTCGTCAAAAAG |
| | | | GCGTATCAGAAACTGTGCTACATTCATCATATCATGGG |
| | | | AAAGGTGCCAGACGCTTGCACTGCCTGCGATCTGGTC |
| | | | AATGTGGATTTGGATGACTGCATCTTTGAACAATAAAT |
| | | | GATTTAAATCAGGTATGGCTGCCGATGGTTATCTTC |
| | | | CAGATTGGCTCGAGGACAACCTTAGTGAAGGAATT |
| | | | CGCGAGTGGTGGGCTTTGAAACCTGGAGCCCCTCA |
| | | | ACCCAAGGCAAATCAACAACATCAAGACAACGCTC |
| | | | GAGGTCTTGTGCTTCCGGGTTACAAATACCTTGGAC |
| | | | CCGGCAACGGACTCGACAAGGGGGAGCCGGTCAA |
| | | | CGCAGCAGACGCGGCGGCCCTCGAGCACGACAAG |
| | | | GCCTACGACCAGCAGCTCAAGGCCGGAGACAACCC |
| | | | GTACCTCAAGTACAACCACGCCGACGCCGAGTTCC |
| | | | AGGAGCGGCTCAAAGAAGATACGTCTTTTGGGGGC |
| | | | AACCTCGGGCGAGCAGTCTTCCAGGCCAAAAAGAG |
| | | | GCTTCTTGAACCTCTTGGTCTGGTTGAGGAAGCGGC |
| | | | TAAGACGGCTCCTGGAAAGAAGAGGCCTGTAGAGC |
| | | | AGTCTCCTCAGGAACCGGACTCCTCCGCGGGTATT |
| | | | GGCAAATCGGGTGCACAGCCCGCTAAAAAGAGACT |
| | | | CAATTTCGGTCAGACTGGCGACACAGAGTCAGTCC |
| | | | CAGACCCTCAACCAATCGGAGAACCTCCCGCAGCC |
| | | | CCCTCAGGTGTGGGATCTCTTACAATGGCTTCAGGT |
| | | | GGTGGCGCACCAGTGGCAGACAATAACGAAGGTG |
| | | | CCGATGGAGTGGGTAGTTCCTCGGGAAATTGGCAT |
| | | | TGCGATTCCCAATGGCTGGGGGACAGAGTCATCAC |
| | | | CACCAGCACCCGAACCTGGGCCCTGCCCACCTACA |
| | | | ACAATCACCTCTACAAGCAAATCTCCAACAGCACA |
| | | | TCTGGAGGATCTTCAAATGACAACGCCTACTTCGG |
| | | | CTACAGCACCCCCTGGGGGTATTTTGACTTCAACA |
| | | | GATTCCACTGCCACTTCTCACCACGTGACTGGCAGC |
| | | | GACTCATCAACAACAACTGGGGATTCCGGCCTAAG |
| | | | CGACTCAACTTCAAGCTCTTCAACATTCAGGTCAA |
| | | | AGAGGTTACGGACAACAATGGAGTCAAGACCATCG |
| | | | CCAATAACCTTACCAGCACGGTCCAGGTCTTCACG |
| | | | GACTCAGACTATCAGCTCCCGTACGTGCTCGGGTC |
| | | | GGCTCACGAGGGCTGCCTCCCGCCGTTCCCAGCGG |
| | | | ACGTTTTCATGATTCCTCAGTACGGGTATCTGACGC |
| | | | TTAATGATGGAAGCCAGGCCGTGGGTCGTTCGTCC |
| | | | TTTTACTGCCTGGAATATTTCCCGTCGCAAATGCTA |
| | | | AGAACGGGTAACAACTTCCAGTTCAGCTACGAGTT |
| | | | TGAGAACGTACCTTTCCATAGCAGCTACGCTCACA |
| | | | GCCAAAGCCTGGACCGACTAATGAATCCACTCATC |
| | | | GACCAATACTTGTACTATCTCTCAAAGACTATTAAC |
| | | | GGTTCTGGACAGAATCAACAAACGCTAAAATTCAG |
| | | | TGTGGCCGGACCCAGCAACATGGCTGTCCAGGGAA |
| | | | GAAACTACATACCTGGACCCAGCTACCGACAACAA |
| | | | CGTGTCTCAACCACTGTGACTCAAAACAACAACAG |
| | | | CGAATTTGCTTGGCCTGGAGCTTCTTCTTGGGCTCT |
| | | | CAATGGACGTAATAGCTTGATGAATCCTGGACCTG |
| | | | CTATGGCCAGCCACAAAGAAGGAGAGGACCGTTTC |
| | | | TTTCCTTTGTCTGGATCTTTAATTTTTGGCAAACAA |
| | | | GGAACTGGAAGAGACAACGTGGATGCGGACAAAG |
| | | | TCATGATAACCAACGAAGAAGAAATTAAAACTACT |
| | | | AACCCGGTAGCAACGGAGTCCTATGGACAAGTGGC |
| | | | CACAAACCACCAGAGTGCCCAAGCACAGGCGCAG |
| | | | ACCGGCTGGGTTCAAAACCAAGGAATACTTCCGGG |
| | | | TATGGTTTGGCAGGACAGAGATGTGTACCTGCAAG |
| | | | GACCCATTTGGGCCAAAATTCCTCACACGGACGGC |
| | | | AACTTTCACCCTTCTCCGCTGATGGGAGGGTTTGGA |
| | | | ATGAAGCACCCGCCTCCTCAGATCCTCATCAAAAA |
| | | | CACACCTGTACCTGCGGATCCTCCAACGGCCTTCA |
| | | | ACAAGGACAAGCTGAACTCTTTCATCACCCAGTAT |
| | | | TCTACTGGCCAAGTCAGCGTGGAGATCGAGTGGGA |
| | | | GCTGCAGAAGGAAAACAGCAAGCGCTGGAACCCG |
| | | | GAGATCCAGTACACTTCCAACTATTACAAGTCTAA |
| | | | TAATGTTGAATTTGCTGTTAATACTGAAGGTGTATA |
| | | | TAGTGAACCCCGCCCCATTGGCACCAGATACCTGA |
| | | | CTCGTAATCTGTAATTGCTTGTTAATCAATAAACC |
| | | | GTTTAATTCGTTTCAGTTGAACTTTGGTCTCTGCG |
| | | | AAGGGCGAATTCGTTTAAACCTGCAGGACTAGAGG |
| | | | TCCTGTATTAGAGGTCACGTGAGTGTTTTGCGA |
| | | | CATTTTGCGACACCATGTGGTCACGCTGGGTAT |
| | | | TTAAGCCCGAGTGAGCACGCAGGGTCTCCATTT |
| | | | TGAAGCGGGAGGTTTGAACGCGCAGCCGCCAAGC |
| | | | CGAATTCTGCAGATCCGGTTAATTCGTTTCAGTTGAAC |
| | | | TTTGGTCTCCTGTCCTTCTTATCTTATCGGTTACCATAG |
| | | | AAACTGGTTACTTATTAACTGCTTGGTGCGCTTCGCGA |
| | | | TAAAAGACTTACGTCATCGGGTTACCCCTAGTGATGGA |
| | | | GCGGCCGCTTTCAGTTGAACTTTGGTCTCTGCGTATTT |
| | | | CTTTCTTATCTAGTTTCCATGCTCTAGAGGTCCTGTA |
| | | | TTAGAGGTCACGTGAGTGTTTTGCGACATTTTG |
| | | | CGACACCATGTGGTCACGCTGGGTATTTAAGCC |
| | | | CGAGTGAGCACGCAGGGTCTCCATTTTGAAGCG |
| | | | GGAGGTTTGAACGCGCAGCCGCCAAGCCGAATTC |
| | | | TGCAGAATCCCAATGGCGCGCCGAGCTTGGCTCGA |
| | | | GCATGGTCATAGCTGTTTCCTGTGTGAAATTGTTAT |
| | | | CCGCTCACAATTCCACACAACATACGAGCCGGAAG |
| | | | CATAAAGTGTAAAGCCTGGGGTGCCTAATGAGTGA |
| | | | GCTAACTCACATTAATTGCGTTGCGCTCACTGCCCG |
| | | | CTTTCCAGTCGGGAAACCTGTCGTGCCAGCTGCATT |
| | | | AATGAATCGGCCAACGCGCGGGGAGAGGCGGTTTG |
| | | | CGTATTGGGCGCTCTTCCGCTTCCTCGCTCACTGAC |
| | | | TCGCTGCGCTCGGTCGTTCGGCTGCGGCGAGCGGT |
| | | | ATCAGCTCACTCAAAGGCGGTAATACGGTTATCCA |
| | | | CAGAATCAGGGGATAACGCAGGAAAGAACATGTG |
| | | | AGCAAAAGGCCAGCAAAAGGCCAGGAACCGTAAA |
| | | | AAGGCCGCGTTGCTGGCGTTTTTCCATAGGCTCCGC |
| | | | CCCCCTGACGAGCATCACAAAAATCGACGCTCAAG |
| | | | TCAGAGGTGGCGAAACCCGACAGGACTATAAAGAT |
| | | | ACCAGGCGTTTCCCCCTGGAAGCTCCCTCGTGCGCT |
| | | | CTCCTGTTCCGACCCTGCCGCTTACCGGATACCTGT |
| | | | CCGCCTTTCTCCCTTCGGGAAGCGTGGCGCTTTCTC |
| | | | ATAGCTCACGCTGTAGGTATCTCAGTTCGGTGTAG |
| | | | GTCGTTCGCTCCAAGCTGGGCTGTGTGCACGAACC |
| | | | CCCCGTTCAGCCCGACCGCTGCGCCTTATCCGGTAA |
| | | | CTATCGTCTTGAGTCCAACCCGGTAAGACACGACT |
| | | | TATCGCCACTGGCAGCAGCCACTGGTAACAGGATT |
| | | | AGCAGAGCGAGGTATGTAGGCGGTGCTACAGAGTT |
| | | | CTTGAAGTGGTGGCCTAACTACGGCTACACTAGAA |
| | | | GAACAGTATTTGGTATCTGCGCTCTGCTGAAGCCA |
| | | | GTTACCTTCGGAAAAAGAGTTGGTAGCTCTTGATC |
| | | | CGGCAAACAAACCACCGCTGGTAGCGGTGGTTTTT |
| | | | TTGTTTGCAAGCAGCAGATTACGCGCAGAAAAAAA |
| | | | GGATCTCAAGAAGATCCTTTGATCTTTTCTACGGGG |
| | | | TCTGACGCTCAGTGGAACGAAAACTCACGTTAAGG |
| | | | GATTTTGGTCATGAGATTATCAAAAAGGATCTTCA |
| | | | CCTAGATCCTTTTAAATTAAAAATGAAGTTTTAAAT |
| | | | CAATCTAAAGTATATATGAGTAAACTTGGTCTGAC |
| | | | AGTTAGAAAAACTCATCGAGCATCAAATGAAACTG |
| | | | CAATTTATTCATATCAGGATTATCAATACCATATTT |
| | | | TTGAAAAAGCCGTTTCTGTAATGAAGGAGAAAACT |
| | | | CACCGAGGCAGTTCCATAGGATGGCAAGATCCTGG |
| | | | TATCGGTCTGCGATTCCGACTCGTCCAACATCAATA |
| | | | CAACCTATTAATTTCCCCTCGTCAAAAATAAGGTTA |
| | | | TCAAGTGAGAAATCACCATGAGTGACGACTGAATC |
| | | | CGGTGAGAATGGCAAAAGTTTATGCATTTCTTTCCA |
| | | | GACTTGTTCAACAGGCCAGCCATTACGCTCGTCATC |
| | | | AAAATCACTCGCATCAACCAAACCGTTATTCATTC |
| | | | GTGATTGCGCCTGAGCGAGACGAAATACGCGATCG |
| | | | CTGTTAAAAGGACAATTACAAACAGGAATCGAATG |
| | | | CAACCGGCGCAGGAACACTGCCAGCGCATCAACAA |
| | | | TATTTTCACCTGAATCAGGATATTCTTCTAATACCT |
| | | | GGAATGCTGTTTTCCCAGGGATCGCAGTGGTGAGT |
| | | | AACCATGCATCATCAGGAGTACGGATAAAATGCTT |
| | | | GATGGTCGGAAGAGGCATAAATTCCGTCAGCCAGT |
| | | | TTAGTCTGACCATCTCATCTGTAACATCATTGGCAA |
| | | | CGCTACCTTTGCCATGTTTCAGAAACAACTCTGGCG |
| | | | CATCGGGCTTCCCATACAATCGATAGATTGTCGCA |
| | | | CCTGATTGCCCGACATTATCGCGAGCCCATTTATAC |
| | | | CCATATAAATCAGCATCCATGTTGGAATTTAATCGC |
| | | | GGCCTAGAGCAAGACGTTTCCCGTTGAATATGGCT |
| | | | CATACTCTTCCTTTTTCAATATTATTGAAGCATTTAT |
| | | | CAGGGTTATTGTCTCATGAGCGGATACATATTTGA |
| | | | ATGTATTTAGAAAAATAAACAAATAGGGGTTCCGC |
| | | | GCACATTTCCCCGAAAAGTGCCACCTGACGTCTAA |
| | | | GAAACCATTATTATCATGACATTAACCTATAAAAA |
| | | | TAGGCGTATCACGAGGCCCTTTCGTC |
| 26 | pYZZ235 | Rep2.Cap9- | atgccggggttttacgagattgtgattaaggtccccagcgaccttgacgagcatct |
| | | p5.L1.p5 | gcccggcatttctgacagctttgtgaactgggtggccgagaaggaatgggagttg |
| | | | ccgccagattctgacatggatctgaatctgattgagcaggcacccctgaccgtggc |
| | | | cgagaagctgcagcgcgactttctgacggaatggcgccgtgtgagtaaggcccc |
| | | | ggaggctcttttctttgtgcaatttgagaagggagagagctacttccacatgcacgt |
| | | | gctcgtggaaaccaccggggtgaaatccatggttttgggacgtttcctgagtcaga |
| | | | ttcgcgaaaaactgattcagagaatttaccgcgggatcgagccgactttgccaaa |
| | | | ctggttcgcggtcacaaagaccagaaatggcgccggaggcgggaacaaggtg |
| | | | gtggatgagtgctacatccccaattacttgctccccaaaacccagcctgagctcca |
| | | | gtgggcgtggactaatatggaacagtatttaagcgcctgtttgaatctcacggagc |
| | | | gtaaacggttggtggcgcagcatctgacgcacgtgtcgcagacgcaggagcag |
| | | | aacaaagagaatcagaatcccaattctgatgcgccggtgatcagatcaaaaact |
| | | | tcagccaggtacatggagctggtcgggtggctcgtggacaaggggattacctcg |
| | | | gagaagcagtggatccaggaggaccaggcctcatacatctccttcaatgcggcct |
| | | | ccaactcgcggtcccaaatcaaggctgccttggacaatgcgggaaagattatga |
| | | | gcctgactaaaaccgcccccgactacctggtgggccagcagcccgtggaggac |
| | | | atttccagcaatcggatttataaaattttggaactaaacgggtacgatccccaatat |
| | | | gcggcttccgtctttctgggatgggccacgaaaaagttcggcaagaggaacacc |
| | | | atctggctgtttgggcctgcaactaccgggaagaccaacatcgcggaggccata |
| | | | gcccacactgtgcccttctacgggtgcgtaaactggaccaatgagaactttcccttc |
| | | | aacgactgtgtcgacaagatggtgatctggtgggaggaggggaagatgaccgc |
| | | | caaggtcgtggagtcggccaaagccattctcggaggaagcaaggtgcgcgtgg |
| | | | accagaaatgcaagtcctcggcccagatagacccgactcccgtgatcgtcacctc |
| | | | caacaccaacatgtgcgccgtgattgacgggaactcaacgaccttcgaacacca |
| | | | gcagccgttgcaagaccggatgttcaaatttgaactcacccgccgtctggatcatg |
| | | | actttgggaaggtcaccaagcaggaagtcaaagactttttccggtgggcaaagg |
| | | | atcacgtggttgaggtggagcatgaattctacgtcaaaaagggtggagccaaga |
| | | | aaagacccgcccccagtgacgcagatataagtgagcccaaacgggtgcgcga |
| | | | gtcagttgcgcagccatcgacgtcagacgcggaagcttcgatcaactacgcaga |
| | | | caggtaccaaaacaaatgttctcgtcacgtgggcatgaatctgatgctgtttccctg |
| | | | cagacaatgcgagagaatgaatcagaattcaaatatctgcttcactcacggaca |
| | | | gaaagactgtttagagtgctttcccgtgtcagaatctcaacccgtttctgtcgtcaaa |
| | | | aaggcgtatcagaaactgtgctacattcatcatatcatgggaaaggtgccagacg |
| | | | cttgcactgcctgcgatctggtcaatgtggatttggatgactgcatctttgaacaata |
| | | | aatgatttaaatcaggtatggctgccgatggttatcttccagattggctcgaggacaac |
| | | | cttagtgaaggaattcgcgagtggtgggctttgaaacctggagcccctcaacccaag |
| | | | gcaaatcaacaacatcaagacaacgctcgaggtcttgtgcttccgggttacaaatacc |
| | | | ttggacccggcaacggactcgacaagggggagccggtcaacgcagcagacgcg |
| | | | gcggccctcgagcacgacaaggcctacgaccagcagctcaaggccggagacaac |
| | | | ccgtacctcaagtacaaccacgccgacgccgagttccaggagcggctcaaagaag |
| | | | atacgtcttttgggggcaacctcgggcgagcagtcttccaggccaaaaagaggcttc |
| | | | ttgaacctcttggtctggttgaggaagcggctaagacggctcctggaaagaagagg |
| | | | cctgtagagcagtctcctcaggaaccggactcctccgcgggtattggcaaatcgggt |
| | | | gcacagcccgctaaaaagagactcaatttcggtcagactggcgacacagagtcagt |
| | | | cccagaccctcaaccaatcggagaacctcccgcagccccctcaggtgtgggatctc |
| | | | ttacaatggcttcaggtggtggcgcaccagtggcagacaataacgaaggtgccgat |
| | | | ggagtgggtagttcctcgggaaattggcattgcgattcccaatggctgggggacaga |
| | | | gtcatcaccaccagcacccgaacctgggccctgcccacctacaacaatcacctctac |
| | | | aagcaaatctccaacagcacatctggaggatcttcaaatgacaacgcctacttcggct |
| | | | acagcaccccctgggggtattttgacttcaacagattccactgccacttctcaccacgt |
| | | | gactggcagcgactcatcaacaacaactggggattccggcctaagcgactcaacttc |
| | | | aagctcttcaacattcaggtcaaagaggttacggacaacaatggagtcaagaccatc |
| | | | gccaataaccttaccagcacggtccaggtcttcacggactcagactatcagctcccgt |
| | | | acgtgctcgggtcggctcacgagggctgcctcccgccgttcccagcggacgttttca |
| | | | tgattcctcagtacgggtatctgacgcttaatgatggaagccaggccgtgggtcgttc |
| | | | gtccttttactgcctggaatatttcccgtcgcaaatgctaagaacgggtaacaacttcc |
| | | | agttcagctacgagtttgagaacgtacctttccatagcagctacgctcacagccaaag |
| | | | cctggaccgactaatgaatccactcatcgaccaatacttgtactatctctcaaagactat |
| | | | taacgcttctggacagaatcaacaaacgctaaaattcagtgtggccggacccagcaa |
| | | | catggctgtccagggaagaaactacatacctggacccagctaccgacaacaacgtg |
| | | | tctcaaccactgtgactcaaaacaacaacagcgaatttgcttggcctggagcttcttctt |
| | | | gggctctcaatggacgtaatagcttgatgaatcctggacctgctatggccagccaca |
| | | | aagaaggagaggaccgtttctttcctttgtctggatctttaatttttggcaaacaaggaa |
| | | | ctggaagagacaacgtggatgcggacaaagtcatgataaccaacgaagaagaaatt |
| | | | aaaactactaacccggtagcaacggagtcctatggacaagtggccacaaaccacca |
| | | | gagtgcccaagcacaggcgcagaccggctgggttcaaaaccaaggaatacttccg |
| | | | ggtatggtttggcaggacagagatgtgtacctgcaaggacccatttgggccaaaatt |
| | | | cctcacacggacggcaactttcacccttctccgctgatgggagggtttggaatgaag |
| | | | cacccgcctcctcagatcctcatcaaaaacacacctgtacctgcggatcctccaacg |
| | | | gccttcaacaaggacaagctgaactctttcatcacccagtattctactggccaagtca |
| | | | gcgtggagatcgagtgggagctgcagaaggaaaacagcaagcgctggaacccgg |
| | | | agatccagtacacttccaactattacaagtctaataatgttgaatttgctgttaatactga |
| | | | aggtgtatatagtgaaccccgccccattggcaccagatacctgactcgtaatctgtaa |
| | | | ttgcttgttaatcaataaaccgtttaattcgtttcagttgaactttggtctctgcgaa |
| | | | gggcgaattcgtttaaacctgcaggactagaggtcctgtattagaggtcacgtgag |
| | | | tgttttgcgacattttgcgacaccatgtggtcacgctgggtatttaagcccgagtg |
| | | | agcacgcagggtctccattttgaagcgggaggtttgaacgcgcagccgccaag |
| | | | ccgaattctgcagatccggttaattcgtttcagttgaactttggtctcctgtccttcttat |
| | | | cttatcggttaccatagaaactggttacttattaactgcttggtgcgcttcgcgataa |
| | | | aagacttacgtcatcgggttacccctagtgatggagcggccgctttcagttgaactt |
| | | | tggtctctgcgtatttctttcttatctagtttccatgctctagaggtcctgtattagaggt |
| | | | cacgtgagtgttttgcgacattttgcgacaccatgtggtcacgctgggtatttaag |
| | | | cccgagtgagcacgcagggtctccattttgaagcgggaggtttgaacgcgcag |
| | | | ccgccaagccgaattctgcagaatcccaatggcgcgccgagcttggctcgagcatg |
| | | | gtcatagctgtttcctgtgtgaaattgttatccgctcacaattccacacaacatacgagc |
| | | | cggaagcataaagtgtaaagcctggggtgcctaatgagtgagctaactcacattaatt |
| | | | gcgttgcgctcactgcccgctttccagtcgggaaacctgtcgtgccagctgcattaat |
| | | | gaatcggccaacgcgcggggagaggcggtttgcgtattgggcgctcttccgcttcct |
| | | | cgctcactgactcgctgcgctcggtcgttcggctgcggcgagcggtatcagctcact |
| | | | caaaggcggtaatacggttatccacagaatcaggggataacgcaggaaagaacat |
| | | | gtgagcaaaaggccagcaaaaggccaggaaccgtaaaaaggccgcgttgctggc |
| | | | gtttttccataggctccgcccccctgacgagcatcacaaaaatcgacgctcaagtcag |
| | | | aggtggcgaaacccgacaggactataaagataccaggcgtttccccctggaagctc |
| | | | cctcgtgcgctctcctgttccgaccctgccgcttaccggatacctgtccgcctttctcc |
| | | | cttcgggaagcgtggcgctttctcatagctcacgctgtaggtatctcagttcggtgtag |
| | | | gtcgttcgctccaagctgggctgtgtgcacgaaccccccgttcagcccgaccgctgc |
| | | | gccttatccggtaactatcgtcttgagtccaacccggtaagacacgacttatcgccact |
| | | | ggcagcagccactggtaacaggattagcagagcgaggtatgtaggcggtgctaca |
| | | | gagttcttgaagtggtggcctaactacggctacactagaagaacagtatttggtatctg |
| | | | cgctctgctgaagccagttaccttcggaaaaagagttggtagctcttgatccggcaaa |
| | | | caaaccaccgctggtagcggtggtttttttgtttgcaagcagcagattacgcgcagaa |
| | | | aaaaaggatctcaagaagatcctttgatcttttctacggggtctgacgctcagtggaac |
| | | | gaaaactcacgttaagggattttggtcatgagattatcaaaaaggatcttcacctagat |
| | | | ccttttaaattaaaaatgaagttttaaatcaatctaaagtatatatgagtaaacttggtctg |
| | | | acagttagaaaaactcatcgagcatcaaatgaaactgcaatttattcatatcaggattat |
| | | | caataccatatttttgaaaaagccgtttctgtaatgaaggagaaaactcaccgaggca |
| | | | gttccataggatggcaagatcctggtatcggtctgcgattccgactcgtccaacatca |
| | | | atacaacctattaatttcccctcgtcaaaaataaggttatcaagtgagaaatcaccatga |
| | | | gtgacgactgaatccggtgagaatggcaaaagtttatgcatttctttccagacttgttca |
| | | | acaggccagccattacgctcgtcatcaaaatcactcgcatcaaccaaaccgttattca |
| | | | ttcgtgattgcgcctgagcgagacgaaatacgcgatcgctgttaaaaggacaattac |
| | | | aaacaggaatcgaatgcaaccggcgcaggaacactgccagcgcatcaacaatattt |
| | | | tcacctgaatcaggatattcttctaatacctggaatgctgttttcccagggatcgcagtg |
| | | | gtgagtaaccatgcatcatcaggagtacggataaaatgcttgatggtcggaagaggc |
| | | | ataaattccgtcagccagtttagtctgaccatctcatctgtaacatcattggcaacgcta |
| | | | cctttgccatgtttcagaaacaactctggcgcatcgggcttcccatacaatcgatagat |
| | | | tgtcgcacctgattgcccgacattatcgcgagcccatttatacccatataaatcagcat |
| | | | ccatgttggaatttaatcgcggcctagagcaagacgtttcccgttgaatatggctcata |
| | | | ctcttcctttttcaatattattgaagcatttatcagggttattgtctcatgagcggatacat |
| | | | atttgaatgtatttagaaaaataaacaaataggggttccgcgcacatttccccgaaaagt |
| | | | gccacctgacgtctaagaaaccattattatcatgacattaacctataaaaataggcgta |
| | | | tcacgaggccctttcgtctcgcgcgtttcggtgatgacggtgaaaacctctgacacat |
| | | | gcagctcccggagacggtcacagcttgtctgtaagcggatgccgggagcagacaa |
| | | | gcccgtcagggcgcgtcagcgggtgttggcgggtgtcggggctggcttaactatgc |
| | | | ggcatcagagcagattgtactgagagtgcaccatatgcggtgtgaaataccgcaca |
| | | | gatgcgtaaggagaaaataccgcatcaggcgccattcgccattcaggctgcgcaac |
| | | | tgttgggaagggcgatcggtgcgggcctcttcgctattacgccagctggcgaaagg |
| | | | gggatgtgctgcaaggcgattaagttgggtaacgccagggttttcccagtcacgacg |
| | | | ttgtaaaacgacggccagtgaattgacgcgtattgggatcgcgcagccgcc |
| 27 | pYZZ236 | AltRep2.Cap9- | TCGCGCGTTTCGGTGATGACGGTGAAAACCTCTGA |
| | | p5.L1.p5 | CACATGCAGCTCCCGGAGACGGTCACAGCTTGTCT |
| | | | GTAAGCGGATGCCGGGAGCAGACAAGCCCGTCAG |
| | | | GGCGCGTCAGCGGGTGTTGGCGGGTGTCGGGGCTG |
| | | | GCTTAACTATGCGGCATCAGAGCAGATTGTACTGA |
| | | | GAGTGCACCATATGCGGTGTGAAATACCGCACAGA |
| | | | TGCGTAAGGAGAAAATACCGCATCAGGCGCCATTC |
| | | | GCCATTCAGGCTGCGCAACTGTTGGGAAGGGCGAT |
| | | | CGGTGCGGGCCTCTTCGCTATTACGCCAGCTGGCG |
| | | | AAAGGGGGATGTGCTGCAAGGCGATTAAGTTGGGT |
| | | | AACGCCAGGGTTTTCCCAGTCACGACGTTGTAAAA |
| | | | CGACGGCCAGTGAATTGACGCGTATTGGGATCGCG |
| | | | CAGCCGCCACGCCGGGGTTTTACGAGATTGTGATTAA |
| | | | GGTCCCCAGCGACCTTGACGAGCATCTGCCCGGCATT |
| | | | TCTGACAGCTTTGTGAACTGGGTGGCCGAGAAGGAAT |
| | | | GGGAGTTGCCGCCAGATTCTGACATGGATCTGAATCT |
| | | | GATTGAGCAGGCACCCCTGACCGTGGCCGAGAAGCT |
| | | | GCAGCGCGACTTTCTGACGGAATGGCGCCGTGTGAGT |
| | | | AAGGCCCCGGAGGCTCTTTTCTTTGTGCAATTTGAGAA |
| | | | GGGAGAGAGCTACTTCCACATGCACGTGCTCGTGGAA |
| | | | ACCACCGGGGTGAAATCCATGGTTTTGGGACGTTTCCT |
| | | | GAGTCAGATTCGCGAAAAACTGATTCAGAGAATTTACC |
| | | | GCGGGATCGAGCCGACTTTGCCAAACTGGTTCGCGGT |
| | | | CACAAAGACCAGAAATGGCGCCGGAGGCGGGAACAA |
| | | | GGTGGTGGATGAGTGCTACATCCCCAATTACTTGCTCC |
| | | | CCAAAACCCAGCCTGAGCTCCAGTGGGCGTGGACTAA |
| | | | TATGGAACAGTATTTAAGCGCCTGTTTGAATCTCACGG |
| | | | AGCGTAAACGGTTGGTGGCGCAGCATCTGACGCACGT |
| | | | GTCGCAGACGCAGGAGCAGAACAAAGAGAATCAGAAT |
| | | | CCCAATTCTGATGCGCCGGTGATCAGATCAAAAACTTC |
| | | | AGCCAGGTACATGGAGCTGGTCGGGTGGCTCGTGGA |
| | | | CAAGGGGATTACCTCGGAGAAGCAGTGGATCCAGGAG |
| | | | GACCAGGCCTCATACATCTCCTTCAATGCGGCCTCCAA |
| | | | CTCGCGGTCCCAAATCAAGGCTGCCTTGGACAATGCG |
| | | | GGAAAGATTATGAGCCTGACTAAAACCGCCCCCGACT |
| | | | ACCTGGTGGGCCAGCAGCCCGTGGAGGACATTTCCAG |
| | | | CAATCGGATTTATAAAATTTTGGAACTAAACGGGTACG |
| | | | ATCCCCAATATGCGGCTTCCGTCTTTCTGGGATGGGC |
| | | | CACGAAAAAGTTCGGCAAGAGGAACACCATCTGGCTG |
| | | | TTTGGGCCTGCAACTACCGGGAAGACCAACATCGCGG |
| | | | AGGCCATAGCCCACACTGTGCCCTTCTACGGGTGCGT |
| | | | AAACTGGACCAATGAGAACTTTCCCTTCAACGACTGTG |
| | | | TCGACAAGATGGTGATCTGGTGGGAGGAGGGGAAGAT |
| | | | GACCGCCAAGGTCGTGGAGTCGGCCAAAGCCATTCTC |
| | | | GGAGGAAGCAAGGTGCGCGTGGACCAGAAATGCAAG |
| | | | TCCTCGGCCCAGATAGACCCGACTCCCGTGATCGTCA |
| | | | CCTCCAACACCAACATGTGCGCCGTGATTGACGGGAA |
| | | | CTCAACGACCTTCGAACACCAGCAGCCGTTGCAAGAC |
| | | | CGGATGTTCAAATTTGAACTCACCCGCCGTCTGGATCA |
| | | | TGACTTTGGGAAGGTCACCAAGCAGGAAGTCAAAGAC |
| | | | TTTTTCCGGTGGGCAAAGGATCACGTGGTTGAGGTGG |
| | | | AGCATGAATTCTACGTCAAAAAGGGTGGAGCCAAGAAA |
| | | | AGACCCGCCCCCAGTGACGCAGATATAAGTGAGCCCA |
| | | | AACGGGTGCGCGAGTCAGTTGCGCAGCCATCGACGTC |
| | | | AGACGCGGAAGCTTCGATCAACTACGCAGACAGGTAC |
| | | | CAAAACAAATGTTCTCGTCACGTGGGCATGAATCTGAT |
| | | | GCTGTTTCCCTGCAGACAATGCGAGAGAATGAATCAGA |
| | | | ATTCAAATATCTGCTTCACTCACGGACAGAAAGACTGT |
| | | | TTAGAGTGCTTTCCCGTGTCAGAATCTCAACCCGTTTC |
| | | | TGTCGTCAAAAAGGCGTATCAGAAACTGTGCTACATTC |
| | | | ATCATATCATGGGAAAGGTGCCAGACGCTTGCACTGC |
| | | | CTGCGATCTGGTCAATGTGGATTTGGATGACTGCATCT |
| | | | TTGAACAATAAATGATTTAAATCAGGTATGGCTGCC |
| | | | GATGGTTATCTTCCAGATTGGCTCGAGGACAACCTT |
| | | | AGTGAAGGAATTCGCGAGTGGTGGGCTTTGAAACC |
| | | | TGGAGCCCCTCAACCCAAGGCAAATCAACAACATC |
| | | | AAGACAACGCTCGAGGTCTTGTGCTTCCGGGTTAC |
| | | | AAATACCTTGGACCCGGCAACGGACTCGACAAGGG |
| | | | GGAGCCGGTCAACGCAGCAGACGCGGCGGCCCTCG |
| | | | AGCACGACAAGGCCTACGACCAGCAGCTCAAGGCC |
| | | | GGAGACAACCCGTACCTCAAGTACAACCACGCCGA |
| | | | CGCCGAGTTCCAGGAGCGGCTCAAAGAAGATACGT |
| | | | CTTTTGGGGGCAACCTCGGGCGAGCAGTCTTCCAG |
| | | | GCCAAAAAGAGGCTTCTTGAACCTCTTGGTCTGGTT |
| | | | GAGGAAGCGGCTAAGACGGCTCCTGGAAAGAAGA |
| | | | GGCCTGTAGAGCAGTCTCCTCAGGAACCGGACTCC |
| | | | TCCGCGGGTATTGGCAAATCGGGTGCACAGCCCGC |
| | | | TAAAAAGAGACTCAATTTCGGTCAGACTGGCGACA |
| | | | CAGAGTCAGTCCCAGACCCTCAACCAATCGGAGAA |
| | | | CCTCCCGCAGCCCCCTCAGGTGTGGGATCTCTTACA |
| | | | ATGGCTTCAGGTGGTGGCGCACCAGTGGCAGACAA |
| | | | TAACGAAGGTGCCGATGGAGTGGGTAGTTCCTCGG |
| | | | GAAATTGGCATTGCGATTCCCAATGGCTGGGGGAC |
| | | | AGAGTCATCACCACCAGCACCCGAACCTGGGCCCT |
| | | | GCCCACCTACAACAATCACCTCTACAAGCAAATCT |
| | | | CCAACAGCACATCTGGAGGATCTTCAAATGACAAC |
| | | | GCCTACTTCGGCTACAGCACCCCCTGGGGGTATTTT |
| | | | GACTTCAACAGATTCCACTGCCACTTCTCACCACGT |
| | | | GACTGGCAGCGACTCATCAACAACAACTGGGGATT |
| | | | CCGGCCTAAGCGACTCAACTTCAAGCTCTTCAACA |
| | | | TTCAGGTCAAAGAGGTTACGGACAACAATGGAGTC |
| | | | AAGACCATCGCCAATAACCTTACCAGCACGGTCCA |
| | | | GGTCTTCACGGACTCAGACTATCAGCTCCCGTACGT |
| | | | GCTCGGGTCGGCTCACGAGGGCTGCCTCCCGCCGT |
| | | | TCCCAGCGGACGTTTTCATGATTCCTCAGTACGGGT |
| | | | ATCTGACGCTTAATGATGGAAGCCAGGCCGTGGGT |
| | | | CGTTCGTCCTTTTACTGCCTGGAATATTTCCCGTCG |
| | | | CAAATGCTAAGAACGGGTAACAACTTCCAGTTCAG |
| | | | CTACGAGTTTGAGAACGTACCTTTCCATAGCAGCT |
| | | | ACGCTCACAGCCAAAGCCTGGACCGACTAATGAAT |
| | | | CCACTCATCGACCAATACTTGTACTATCTCTCAAAG |
| | | | ACTATTAACGGTTCTGGACAGAATCAACAAACGCT |
| | | | AAAATTCAGTGTGGCCGGACCCAGCAACATGGCTG |
| | | | TCCAGGGAAGAAACTACATACCTGGACCCAGCTAC |
| | | | CGACAACAACGTGTCTCAACCACTGTGACTCAAAA |
| | | | CAACAACAGCGAATTTGCTTGGCCTGGAGCTTCTTC |
| | | | TTGGGCTCTCAATGGACGTAATAGCTTGATGAATC |
| | | | CTGGACCTGCTATGGCCAGCCACAAAGAAGGAGAG |
| | | | GACCGTTTCTTTCCTTTGTCTGGATCTTTAATTTTTG |
| | | | GCAAACAAGGAACTGGAAGAGACAACGTGGATGC |
| | | | GGACAAAGTCATGATAACCAACGAAGAAGAAATT |
| | | | AAAACTACTAACCCGGTAGCAACGGAGTCCTATGG |
| | | | ACAAGTGGCCACAAACCACCAGAGTGCCCAAGCAC |
| | | | AGGCGCAGACCGGCTGGGTTCAAAACCAAGGAAT |
| | | | ACTTCCGGGTATGGTTTGGCAGGACAGAGATGTGT |
| | | | ACCTGCAAGGACCCATTTGGGCCAAAATTCCTCAC |
| | | | ACGGACGGCAACTTTCACCCTTCTCCGCTGATGGG |
| | | | AGGGTTTGGAATGAAGCACCCGCCTCCTCAGATCC |
| | | | TCATCAAAAACACACCTGTACCTGCGGATCCTCCA |
| | | | ACGGCCTTCAACAAGGACAAGCTGAACTCTTTCAT |
| | | | CACCCAGTATTCTACTGGCCAAGTCAGCGTGGAGA |
| | | | TCGAGTGGGAGCTGCAGAAGGAAAACAGCAAGCG |
| | | | CTGGAACCCGGAGATCCAGTACACTTCCAACTATT |
| | | | ACAAGTCTAATAATGTTGAATTTGCTGTTAATACTG |
| | | | AAGGTGTATATAGTGAACCCCGCCCCATTGGCACC |
| | | | AGATACCTGACTCGTAATCTGTAATTGCTTGTTAAT |
| | | | CAATAAACCGTTTAATTCGTTTCAGTTGAACTTTGGT |
| | | | CTCTGCGAAGGGCGAATTCGTTTAAACCTGCAGGA |
| | | | CTAGAGGTCCTGTATTAGAGGTCACGTGAGTGT |
| | | | TTTGCGACATTTTGCGACACCATGTGGTCACGC |
| | | | TGGGTATTTAAGCCCGAGTGAGCACGCAGGGTC |
| | | | TCCATTTTGAAGCGGGAGGTTTGAACGCGCAGCC |
| | | | GCCAAGCCGAATTCTGCAGATCCGGTTAATTCGTTTCA |
| | | | GTTGAACTTTGGTCTCCTGTCCTTCTTATCTTATCGGTT |
| | | | ACCATAGAAACTGGTTACTTATTAACTGCTTGGTGCGC |
| | | | TTCGCGATAAAAGACTTACGTCATCGGGTTACCCCTAG |
| | | | TGATGGAGCGGCCGCTTTCAGTTGAACTTTGGTCTCTG |
| | | | CGTATTTCTTTCTTATCTAGTTTCCATGCTCTAGAGGT |
| | | | CCTGTATTAGAGGTCACGTGAGTGTTTTGCGAC |
| | | | ATTTTGCGACACCATGTGGTCACGCTGGGTATT |
| | | | TAAGCCCGAGTGAGCACGCAGGGTCTCCATTTT |
| | | | GAAGCGGGAGGTTTGAACGCGCAGCCGCCAAGCC |
| | | | GAATTCTGCAGAATCCCAATGGCGCGCCGAGCTTG |
| | | | GCTCGAGCATGGTCATAGCTGTTTCCTGTGTGAAAT |
| | | | TGTTATCCGCTCACAATTCCACACAACATACGAGC |
| | | | CGGAAGCATAAAGTGTAAAGCCTGGGGTGCCTAAT |
| | | | GAGTGAGCTAACTCACATTAATTGCGTTGCGCTCA |
| | | | CTGCCCGCTTTCCAGTCGGGAAACCTGTCGTGCCA |
| | | | GCTGCATTAATGAATCGGCCAACGCGCGGGGAGAG |
| | | | GCGGTTTGCGTATTGGGCGCTCTTCCGCTTCCTCGC |
| | | | TCACTGACTCGCTGCGCTCGGTCGTTCGGCTGCGGC |
| | | | GAGCGGTATCAGCTCACTCAAAGGCGGTAATACGG |
| | | | TTATCCACAGAATCAGGGGATAACGCAGGAAAGA |
| | | | ACATGTGAGCAAAAGGCCAGCAAAAGGCCAGGAA |
| | | | CCGTAAAAAGGCCGCGTTGCTGGCGTTTTTCCATA |
| | | | GGCTCCGCCCCCCTGACGAGCATCACAAAAATCGA |
| | | | CGCTCAAGTCAGAGGTGGCGAAACCCGACAGGACT |
| | | | ATAAAGATACCAGGCGTTTCCCCCTGGAAGCTCCC |
| | | | TCGTGCGCTCTCCTGTTCCGACCCTGCCGCTTACCG |
| | | | GATACCTGTCCGCCTTTCTCCCTTCGGGAAGCGTGG |
| | | | CGCTTTCTCATAGCTCACGCTGTAGGTATCTCAGTT |
| | | | CGGTGTAGGTCGTTCGCTCCAAGCTGGGCTGTGTG |
| | | | CACGAACCCCCCGTTCAGCCCGACCGCTGCGCCTT |
| | | | ATCCGGTAACTATCGTCTTGAGTCCAACCCGGTAA |
| | | | GACACGACTTATCGCCACTGGCAGCAGCCACTGGT |
| | | | AACAGGATTAGCAGAGCGAGGTATGTAGGCGGTGC |
| | | | TACAGAGTTCTTGAAGTGGTGGCCTAACTACGGCT |
| | | | ACACTAGAAGAACAGTATTTGGTATCTGCGCTCTG |
| | | | CTGAAGCCAGTTACCTTCGGAAAAAGAGTTGGTAG |
| | | | CTCTTGATCCGGCAAACAAACCACCGCTGGTAGCG |
| | | | GTGGTTTTTTTGTTTGCAAGCAGCAGATTACGCGCA |
| | | | GAAAAAAAGGATCTCAAGAAGATCCTTTGATCTTT |
| | | | TCTACGGGGTCTGACGCTCAGTGGAACGAAAACTC |
| | | | ACGTTAAGGGATTTTGGTCATGAGATTATCAAAAA |
| | | | GGATCTTCACCTAGATCCTTTTAAATTAAAAATGAA |
| | | | GTTTTAAATCAATCTAAAGTATATATGAGTAAACTT |
| | | | GGTCTGACAGTTAGAAAAACTCATCGAGCATCAAA |
| | | | TGAAACTGCAATTTATTCATATCAGGATTATCAATA |
| | | | CCATATTTTTGAAAAAGCCGTTTCTGTAATGAAGG |
| | | | AGAAAACTCACCGAGGCAGTTCCATAGGATGGCAA |
| | | | GATCCTGGTATCGGTCTGCGATTCCGACTCGTCCAA |
| | | | CATCAATACAACCTATTAATTTCCCCTCGTCAAAAA |
| | | | TAAGGTTATCAAGTGAGAAATCACCATGAGTGACG |
| | | | ACTGAATCCGGTGAGAATGGCAAAAGTTTATGCAT |
| | | | TTCTTTCCAGACTTGTTCAACAGGCCAGCCATTACG |
| | | | CTCGTCATCAAAATCACTCGCATCAACCAAACCGT |
| | | | TATTCATTCGTGATTGCGCCTGAGCGAGACGAAAT |
| | | | ACGCGATCGCTGTTAAAAGGACAATTACAAACAGG |
| | | | AATCGAATGCAACCGGCGCAGGAACACTGCCAGCG |
| | | | CATCAACAATATTTTCACCTGAATCAGGATATTCTT |
| | | | CTAATACCTGGAATGCTGTTTTCCCAGGGATCGCA |
| | | | GTGGTGAGTAACCATGCATCATCAGGAGTACGGAT |
| | | | AAAATGCTTGATGGTCGGAAGAGGCATAAATTCCG |
| | | | TCAGCCAGTTTAGTCTGACCATCTCATCTGTAACAT |
| | | | CATTGGCAACGCTACCTTTGCCATGTTTCAGAAACA |
| | | | ACTCTGGCGCATCGGGCTTCCCATACAATCGATAG |
| | | | ATTGTCGCACCTGATTGCCCGACATTATCGCGAGCC |
| | | | CATTTATACCCATATAAATCAGCATCCATGTTGGAA |
| | | | TTTAATCGCGGCCTAGAGCAAGACGTTTCCCGTTG |
| | | | AATATGGCTCATACTCTTCCTTTTTCAATATTATTG |
| | | | AAGCATTTATCAGGGTTATTGTCTCATGAGCGGAT |
| | | | ACATATTTGAATGTATTTAGAAAAATAAACAAATA |
| | | | GGGGTTCCGCGCACATTTCCCCGAAAAGTGCCACC |
| | | | TGACGTCTAAGAAACCATTATTATCATGACATTAA |
| | | | CCTATAAAAATAGGCGTATCACGAGGCCCTTTCGT |
| | | | C |
| 28 | pYZZ408 | Rep2Cap9- | atgccggggttttacgagattgtgattaaggtccccagcgaccttgacgagcatct |
| | | p5.L2.p5 | gcccggcatttctgacagctttgtgaactgggtggccgagaaggaatgggagttg |
| | | | ccgccagattctgacatggatctgaatctgattgagcaggcacccctgaccgtggc |
| | | | cgagaagctgcagcgcgactttctgacggaatggcgccgtgtgagtaaggcccc |
| | | | ggaggctcttttctttgtgcaatttgagaagggagagagctacttccacatgcacgt |
| | | | gctcgtggaaaccaccggggtgaaatccatggttttgggacgtttcctgagtcaga |
| | | | ttcgcgaaaaactgattcagagaatttaccggggatcgagccgactttgccaaa |
| | | | ctggttcgcggtcacaaagaccagaaatggcgccggaggcgggaacaaggtg |
| | | | gtggatgagtgctacatccccaattacttgctccccaaaacccagcctgagctcca |
| | | | gtgggcgtggactaatatggaacagtatttaagcgcctgtttgaatctcacggagc |
| | | | gtaaacggttggtggcgcagcatctgacgcacgtgtcgcagacgcaggagcag |
| | | | aacaaagagaatcagaatcccaattctgatgcgccggtgatcagatcaaaaact |
| | | | tcagccaggtacatggagctggtcgggtggctcgtggacaaggggattacctcg |
| | | | gagaagcagtggatccaggaggaccaggcctcatacatctccttcaatgcggcct |
| | | | ccaactcgcggtcccaaatcaaggctgccttggacaatgcgggaaagattatga |
| | | | gcctgactaaaaccgcccccgactacctggtgggccagcagcccgtggaggac |
| | | | atttccagcaatcggatttataaaattttggaactaaacgggtacgatccccaatat |
| | | | gcggcttccgtctttctgggatgggccacgaaaaagttcggcaagaggaacacc |
| | | | atctggctgtttgggcctgcaactaccgggaagaccaacatcgcggaggccata |
| | | | gcccacactgtgcccttctacgggtgcgtaaactggaccaatgagaactttcccttc |
| | | | aacgactgtgtcgacaagatggtgatctggtgggaggaggggaagatgaccgc |
| | | | caaggtcgtggagtcggccaaagccattctcggaggaagcaaggtgcgcgtgg |
| | | | accagaaatgcaagtcctcggcccagatagacccgactcccgtgatcgtcacctc |
| | | | caacaccaacatgtgcgccgtgattgacgggaactcaacgaccttcgaacacca |
| | | | gcagccgttgcaagaccggatgttcaaatttgaactcacccgccgtctggatcatg |
| | | | actttgggaaggtcaccaagcaggaagtcaaagactttttccgggggcaaagg |
| | | | atcacgtggttgaggtggagcatgaattctacgtcaaaaagggtggagccaaga |
| | | | aaagacccgcccccagtgacgcagatataagtgagcccaaacgggtgcgcga |
| | | | gtcagttgcgcagccatcgacgtcagacgcggaagcttcgatcaactacgcaga |
| | | | caggtaccaaaacaaatgttctcgtcacgtgggcatgaatctgatgctgtttccctg |
| | | | cagacaatgcgagagaatgaatcagaattcaaatatctgcttcactcacggaca |
| | | | gaaagactgtttagagtgctttcccgtgtcagaatctcaacccgtttctgtcgtcaaa |
| | | | aaggcgtatcagaaactgtgctacattcatcatatcatgggaaaggtgccagacg |
| | | | cttgcactgcctgcgatctggtcaatgtggatttggatgactgcatctttgaacaata |
| | | | aatgatttaaatcaggtatggctgccgatggttatcttccagattggctcgaggac |
| | | | aaccttagtgaaggaattcgcgagtggtgggctttgaaacctggagcccctcaa |
| | | | cccaaggcaaatcaacaacatcaagacaacgctcgaggtcttgtgcttccggg |
| | | | ttacaaataccttggacccggcaacggactcgacaagggggagccggtcaac |
| | | | gcagcagacgcggcggccctcgagcacgacaaggcctacgaccagcagctca |
| | | | aggccggagacaacccgtacctcaagtacaaccacgccgacgccgagttcca |
| | | | ggagcggctcaaagaagatacgtcttttgggggcaacctcgggcgagcagtct |
| | | | tccaggccaaaaagaggcttcttgaacctcttggtctggttgaggaagcggcta |
| | | | agacggctcctggaaagaagaggcctgtagagcagtctcctcaggaaccgga |
| | | | ctcctccgcgggtattggcaaatcgggtgcacagcccgctaaaaagagactca |
| | | | atttcggtcagactggcgacacagagtcagtcccagaccctcaaccaatcgga |
| | | | gaacctcccgcagccccctcaggtgtgggatctcttacaatggcttcaggtggtg |
| | | | gcgcaccagtggcagacaataacgaaggtgccgatggagtgggtagttcctcg |
| | | | ggaaattggcattgcgattcccaatggctgggggacagagtcatcaccaccag |
| | | | cacccgaacctgggccctgcccacctacaacaatcacctctacaagcaaatctc |
| | | | caacagcacatctggaggatcttcaaatgacaacgcctacttcggctacagcac |
| | | | cccctgggggtattttgacttcaacagattccactgccacttctcaccacgtgact |
| | | | ggcagcgactcatcaacaacaactggggattccggcctaagcgactcaacttc |
| | | | aagctcttcaacattcaggtcaaagaggttacggacaacaatggagtcaagac |
| | | | catcgccaataaccttaccagcacggtccaggtcttcacggactcagactatca |
| | | | gctcccgtacgtgctcgggtcggctcacgagggctgcctcccgccgttcccagc |
| | | | ggacgttttcatgattcctcagtacgggtatctgacgcttaatgatggaagccag |
| | | | gccgtgggtcgttcgtccttttactgcctggaatatttcccgtcgcaaatgctaag |
| | | | aacgggtaacaacttccagttcagctacgagtttgagaacgtacctttccatagc |
| | | | agctacgctcacagccaaagcctggaccgactaatgaatccactcatcgacca |
| | | | atacttgtactatctctcaaagactattaacggttctggacagaatcaacaaacg |
| | | | ctaaaattcagtgtggccggacccagcaacatggctgtccagggaagaaacta |
| | | | catacctggacccagctaccgacaacaacgtgtctcaaccactgtgactcaaa |
| | | | acaacaacagcgaatttgcttggcctggagcttcttcttgggctctcaatggacg |
| | | | taatagcttgatgaatcctggacctgctatggccagccacaaagaaggagagg |
| | | | accgtttctttcctttgtctggatctttaatttttggcaaacaaggaactggaaga |
| | | | gacaacgtggatgcggacaaagtcatgataaccaacgaagaagaaattaaa |
| | | | actactaacccggtagcaacggagtcctatggacaagtggccacaaaccacca |
| | | | gagtgcccaagcacaggcgcagaccggctgggttcaaaaccaaggaatactt |
| | | | ccgggtatggtttggcaggacagagatgtgtacctgcaaggacccatttgggcc |
| | | | aaaattcctcacacggacggcaactttcacccttctccgctgatgggagggtttg |
| | | | gaatgaagcacccgcctcctcagatcctcatcaaaaacacacctgtacctgcgg |
| | | | atcctccaacggccttcaacaaggacaagctgaactctttcatcacccagtattc |
| | | | tactggccaagtcagcgtggagatcgagtgggagctgcagaaggaaaacagc |
| | | | aagcgctggaacccggagatccagtacacttccaactattacaagtctaataat |
| | | | gttgaatttgctgttaatactgaaggtgtatatagtgaaccccgccccattggca |
| | | | ccagatacctgactcgtaatctgtaattgcttgttaatcaataaaccgtttaattc |
| | | | gtttcagttgaACTTTGGTCTCTGCCCTAGGGGAGGTCC |
| | | | TGTATTAGAGGTCACGTGAGTGTTTTGCGACAT |
| | | | TTTGCGACACCATGTGGTCACGCTGGGTATTTA |
| | | | AGCCCGAGTGAGCACGCAGGGTCTCCATTTTGA |
| | | | AGCGGGAGGTTTGAAcgcgcagccgccaagccgtttcagttgaac |
| | | | tttggtctctgcgtatttctttcttatctagtttccatggaggtcctgtattagaggtcac |
| | | | gtgagtgttttgcgacattttgcgacaccatgtggtcacgctgggtatttaagccc |
| | | | gagtgagcacgcagggtctccattttgaagcgggaggtttgaaATCCCAA |
| | | | TGGCGCGCCGAGCTTGGCTCGAGCATGGTCATAGC |
| | | | TGTTTCCTGTGTGAAATTGTTATCCGCTCACAATTC |
| | | | CACACAACATACGAGCCGGAAGCATAAAGTGTAA |
| | | | AGCCTGGGGTGCCTAATGAGTGAGCTAACTCACAT |
| | | | TAATTGCGTTGCGCTCACTGCCCGCTTTCCAGTCGG |
| | | | GAAACCTGTCGTGCCAGCTGCATTAATGAATCGGC |
| | | | CAACGCGCGGGGAGAGGCGGTTTGCGTATTGGGCG |
| | | | CTCTTCCGCTTCCTCGCTCACTGACTCGCTGCGCTC |
| | | | GGTCGTTCGGCTGCGGCGAGCGGTATCAGCTCACT |
| | | | CAAAGGCGGTAATACGGTTATCCACAGAATCAGGG |
| | | | GATAACGCAGGAAAGAACATGTGAGCAAAAGGCC |
| | | | AGCAAAAGGCCAGGAACCGTAAAAAGGCCGCGTT |
| | | | GCTGGCGTTTTTCCATAGGCTCCGCCCCCCTGACGA |
| | | | GCATCACAAAAATCGACGCTCAAGTCAGAGGTGGC |
| | | | GAAACCCGACAGGACTATAAAGATACCAGGCGTTT |
| | | | CCCCCTGGAAGCTCCCTCGTGCGCTCTCCTGTTCCG |
| | | | ACCCTGCCGCTTACCGGATACCTGTCCGCCTTTCTC |
| | | | CCTTCGGGAAGCGTGGCGCTTTCTCATAGCTCACGC |
| | | | TGTAGGTATCTCAGTTCGGTGTAGGTCGTTCGCTCC |
| | | | AAGCTGGGCTGTGTGCACGAACCCCCCGTTCAGCC |
| | | | CGACCGCTGCGCCTTATCCGGTAACTATCGTCTTGA |
| | | | GTCCAACCCGGTAAGACACGACTTATCGCCACTGG |
| | | | CAGCAGCCACTGGTAACAGGATTAGCAGAGCGAG |
| | | | GTATGTAGGCGGTGCTACAGAGTTCTTGAAGTGGT |
| | | | GGCCTAACTACGGCTACACTAGAAGAACAGTATTT |
| | | | GGTATCTGCGCTCTGCTGAAGCCAGTTACCTTCGGA |
| | | | AAAAGAGTTGGTAGCTCTTGATCCGGCAAACAAAC |
| | | | CACCGCTGGTAGCGGTGGTTTTTTTGTTTGCAAGCA |
| | | | GCAGATTACGCGCAGAAAAAAAGGATCTCAAGAA |
| | | | GATCCTTTGATCTTTTCTACGGGGTCTGACGCTCAG |
| | | | TGGAACGAAAACTCACGTTAAGGGATTTTGGTCAT |
| | | | GAGATTATCAAAAAGGATCTTCACCTAGATCCTTTT |
| | | | AAATTAAAAATGAAGTTTTAAATCAATCTAAAGTA |
| | | | TATATGAGTAAACTTGGTCTGACAGTTAGAAAAAC |
| | | | TCATCGAGCATCAAATGAAACTGCAATTTATTCAT |
| | | | ATCAGGATTATCAATACCATATTTTTGAAAAAGCC |
| | | | GTTTCTGTAATGAAGGAGAAAACTCACCGAGGCAG |
| | | | TTCCATAGGATGGCAAGATCCTGGTATCGGTCTGC |
| | | | GATTCCGACTCGTCCAACATCAATACAACCTATTA |
| | | | ATTTCCCCTCGTCAAAAATAAGGTTATCAAGTGAG |
| | | | AAATCACCATGAGTGACGACTGAATCCGGTGAGAA |
| | | | TGGCAAAAGTTTATGCATTTCTTTCCAGACTTGTTC |
| | | | AACAGGCCAGCCATTACGCTCGTCATCAAAATCAC |
| | | | TCGCATCAACCAAACCGTTATTCATTCGTGATTGCG |
| | | | CCTGAGCGAGACGAAATACGCGATCGCTGTTAAAA |
| | | | GGACAATTACAAACAGGAATCGAATGCAACCGGC |
| | | | GCAGGAACACTGCCAGCGCATCAACAATATTTTCA |
| | | | CCTGAATCAGGATATTCTTCTAATACCTGGAATGCT |
| | | | GTTTTCCCAGGGATCGCAGTGGTGAGTAACCATGC |
| | | | ATCATCAGGAGTACGGATAAAATGCTTGATGGTCG |
| | | | GAAGAGGCATAAATTCCGTCAGCCAGTTTAGTCTG |
| | | | ACCATCTCATCTGTAACATCATTGGCAACGCTACCT |
| | | | TTGCCATGTTTCAGAAACAACTCTGGCGCATCGGG |
| | | | CTTCCCATACAATCGATAGATTGTCGCACCTGATTG |
| | | | CCCGACATTATCGCGAGCCCATTTATACCCATATAA |
| | | | ATCAGCATCCATGTTGGAATTTAATCGCGGCCTAG |
| | | | AGCAAGACGTTTCCCGTTGAATATGGCTCATACTCT |
| | | | TCCTTTTTCAATATTATTGAAGCATTTATCAGGGTT |
| | | | ATTGTCTCATGAGCGGATACATATTTGAATGTATTT |
| | | | AGAAAAATAAACAAATAGGGGTTCCGCGCACATTT |
| | | | CCCCGAAAAGTGCCACCTGACGTCTAAGAAACCAT |
| | | | TATTATCATGACATTAACCTATAAAAATAGGCGTA |
| | | | TCACGAGGCCCTTTCGTCTCGCGCGTTTCGGTGATG |
| | | | ACGGTGAAAACCTCTGACACATGCAGCTCCCGGAG |
| | | | ACGGTCACAGCTTGTCTGTAAGCGGATGCCGGGAG |
| | | | CAGACAAGCCCGTCAGGGCGCGTCAGCGGGTGTTG |
| | | | GCGGGTGTCGGGGCTGGCTTAACTATGCGGCATCA |
| | | | GAGCAGATTGTACTGAGAGTGCACCATATGCGGTG |
| | | | TGAAATACCGCACAGATGCGTAAGGAGAAAATACC |
| | | | GCATCAGGCGCCATTCGCCATTCAGGCTGCGCAAC |
| | | | TGTTGGGAAGGGCGATCGGTGCGGGCCTCTTCGCT |
| | | | ATTACGCCAGCTGGCGAAAGGGGGATGTGCTGCAA |
| | | | GGCGATTAAGTTGGGTAACGCCAGGGTTTTCCCAG |
| | | | TCACGACGTTGTAAAACGACGGCCAGTGAATTGAC |
| | | | GCGTATTGGGATCGCGCAGCCGCC |
| 29 | pYZZ409 | Rep2.Cap9- | atgccggggttttacgagattgtgattaaggtccccagcgaccttgacgagcatct |
| | | p5.L3.p5 | gcccggcatttctgacagctttgtgaactgggtggccgagaaggaatgggagttg |
| | | | ccgccagattctgacatggatctgaatctgattgagcaggcacccctgaccgtggc |
| | | | cgagaagctgcagcgcgactttctgacggaatggcgccgtgtgagtaaggcccc |
| | | | ggaggctcttttctttgtgcaatttgagaagggagagagctacttccacatgcacgt |
| | | | gctcgtggaaaccaccggggtgaaatccatggttttgggacgtttcctgagtcaga |
| | | | ttcgcgaaaaactgattcagagaatttaccgcgggatcgagccgactttgccaaa |
| | | | ctggttcgcggtcacaaagaccagaaatggcgccggagggggaacaaggtg |
| | | | gtggatgagtgctacatccccaattacttgctccccaaaacccagcctgagctcca |
| | | | gtgggcgtggactaatatggaacagtatttaagcgcctgtttgaatctcacggagc |
| | | | gtaaacggttggtggcgcagcatctgacgcacgtgtcgcagacgcaggagcag |
| | | | aacaaagagaatcagaatcccaattctgatgcgccggtgatcagatcaaaaact |
| | | | tcagccaggtacatggagctggtcgggtggctcgtggacaaggggattacctcg |
| | | | gagaagcagtggatccaggaggaccaggcctcatacatctccttcaatgcggcct |
| | | | ccaactcgcggtcccaaatcaaggctgccttggacaatgcgggaaagattatga |
| | | | gcctgactaaaaccgcccccgactacctgggggccagcagcccgtggaggac |
| | | | atttccagcaatcggatttataaaattttggaactaaacgggtacgatccccaatat |
| | | | gcggcttccgtctttctgggatgggccacgaaaaagttcggcaagaggaacacc |
| | | | atctggctgtttgggcctgcaactaccgggaagaccaacatcgcggaggccata |
| | | | gcccacactgtgcccttctacgggtgcgtaaactggaccaatgagaactttcccttc |
| | | | aacgactgtgtcgacaagatggtgatctggtgggaggaggggaagatgaccgc |
| | | | caaggtcgtggagtcggccaaagccattctcggaggaagcaaggtgcgcgtgg |
| | | | accagaaatgcaagtcctcggcccagatagacccgactcccgtgatcgtcacctc |
| | | | caacaccaacatgtgcgccgtgattgacgggaactcaacgaccttcgaacacca |
| | | | gcagccgttgcaagaccggatgttcaaatttgaactcacccgccgtctggatcatg |
| | | | actttgggaaggtcaccaagcaggaagtcaaagactttttccggtgggcaaagg |
| | | | atcacgtggttgaggtggagcatgaattctacgtcaaaaagggtggagccaaga |
| | | | aaagacccgcccccagtgacgcagatataagtgagcccaaacgggtgcgcga |
| | | | gtcagttgcgcagccatcgacgtcagacgcggaagcttcgatcaactacgcaga |
| | | | caggtaccaaaacaaatgttctcgtcacgtgggcatgaatctgatgctgtttccctg |
| | | | cagacaatgcgagagaatgaatcagaattcaaatatctgcttcactcacggaca |
| | | | gaaagactgtttagagtgctttcccgtgtcagaatctcaacccgtttctgtcgtcaaa |
| | | | aaggcgtatcagaaactgtgctacattcatcatatcatgggaaaggtgccagacg |
| | | | cttgcactgcctgcgatctggtcaatgtggatttggatgactgcatctttgaacaata |
| | | | aatgatttaaatcaggtatggctgccgatggttatcttccagattggctcgaggacaac |
| | | | cttagtgaaggaattcgcgagtggtgggctttgaaacctggagcccctcaacccaag |
| | | | gcaaatcaacaacatcaagacaacgctcgaggtcttgtgcttccgggttacaaatacc |
| | | | ttggacccggcaacggactcgacaagggggagccggtcaacgcagcagacgcg |
| | | | gcggccctcgagcacgacaaggcctacgaccagcagctcaaggccggagacaac |
| | | | ccgtacctcaagtacaaccacgccgacgccgagttccaggagcggctcaaagaag |
| | | | atacgtcttttgggggcaacctcgggcgagcagtcttccaggccaaaaagaggcttc |
| | | | ttgaacctcttggtctggttgaggaagcggctaagacggctcctggaaagaagagg |
| | | | cctgtagagcagtctcctcaggaaccggactcctccgcgggtattggcaaatcgggt |
| | | | gcacagcccgctaaaaagagactcaatttcggtcagactggcgacacagagtcagt |
| | | | cccagaccctcaaccaatcggagaacctcccgcagccccctcaggtgtgggatctc |
| | | | ttacaatggcttcaggtggtggcgcaccagtggcagacaataacgaaggtgccgat |
| | | | ggagtgggtagttcctcgggaaattggcattgcgattcccaatggctgggggacaga |
| | | | gtcatcaccaccagcacccgaacctgggccctgcccacctacaacaatcacctctac |
| | | | aagcaaatctccaacagcacatctggaggatcttcaaatgacaacgcctacttcggct |
| | | | acagcaccccctgggggtattttgacttcaacagattccactgccacttctcaccacgt |
| | | | gactggcagcgactcatcaacaacaactggggattccggcctaagcgactcaacttc |
| | | | aagctcttcaacattcaggtcaaagaggttacggacaacaatggagtcaagaccatc |
| | | | gccaataaccttaccagcacggtccaggtcttcacggactcagactatcagctcccgt |
| | | | acgtgctcgggtcggctcacgagggctgcctcccgccgttcccagcggacgttttca |
| | | | tgattcctcagtacgggtatctgacgcttaatgatggaagccaggccgtgggtcgttc |
| | | | gtccttttactgcctggaatatttcccgtcgcaaatgctaagaacgggtaacaacttcc |
| | | | agttcagctacgagtttgagaacgtacctttccatagcagctacgctcacagccaaag |
| | | | cctggaccgactaatgaatccactcatcgaccaatacttgtactatctctcaaagactat |
| | | | taacggttctggacagaatcaacaaacgctaaaattcagtgtggccggacccagcaa |
| | | | catggctgtccagggaagaaactacatacctggacccagctaccgacaacaacgtg |
| | | | tctcaaccactgtgactcaaaacaacaacagcgaatttgcttggcctggagcttcttctt |
| | | | gggctctcaatggacgtaatagcttgatgaatcctggacctgctatggccagccaca |
| | | | aagaaggagaggaccgtttctttcctttctctggatctttaatttttggcaaacaaggaa |
| | | | ctggaagagacaacgtggatgcggacaaagtcatgataaccaacgaagaagaaatt |
| | | | aaaactactaacccggtagcaacggagtcctatggacaagtggccacaaaccacca |
| | | | gagtgcccaagcacaggcgcagaccggctgggttcaaaaccaaggaatacttccg |
| | | | ggtatggtttggcaggacagagatgtgtacctgcaaggacccatttgggccaaaatt |
| | | | cctcacacggacggcaactttcacccttctccgctgatgggagggtttggaatgaag |
| | | | cacccgcctcctcagatcctcatcaaaaacacacctgtacctgcggatcctccaacg |
| | | | gccttcaacaaggacaagctgaactctttcatcacccagtattctactggccaagtca |
| | | | gcgtggagatcgagtgggagctgcagaaggaaaacagcaagcgctggaacccgg |
| | | | agatccagtacacttccaactattacaagtctaataatgttgaatttgctgttaatactga |
| | | | aggtgtatatagtgaaccccgccccattggcaccagatacctgactcgtaatctgtaa |
| | | | ttgcttgttaatcaataaaccgtttaattcgtttcagttgaACTTTGGTCTC |
| | | | TGCCCTAGGGGAGGTCCTGTATTAGAGGTCACGT |
| | | | GAGTGTTTTGCGACATTTTGCGACACCATGTGG |
| | | | TCACGCTGGGTATTTAAGCCCGAGTGAGCACGC |
| | | | AGGGTCTCCATTTTGAAGCGGGAGGTTTGAAGT |
| | | | CTTACCCCCAATAAGGGGATTTGCTCTATTTAATTAGGA |
| | | | ATAAGGTCGATTACTGATAGAACAAATCCAGGCTACTG |
| | | | TGTTTAGTAATCAGATTTGTTgaggtcctgtattagaggtcacgtga |
| | | | gtgttttgcgacattttgcgacaccatgtggtcacgctgggtatttaagcccgagt |
| | | | gagcacgcagggtctccattttgaagcgggaggtttgaaATCCCAATG |
| | | | GCGCGCCGAGCTTGGCTCGAGCATGGTCATAGCTG |
| | | | TTTCCTGTGTGAAATTGTTATCCGCTCACAATTCCA |
| | | | CACAACATACGAGCCGGAAGCATAAAGTGTAAAG |
| | | | CCTGGGGTGCCTAATGAGTGAGCTAACTCACATTA |
| | | | ATTGCGTTGCGCTCACTGCCCGCTTTCCAGTCGGGA |
| | | | AACCTGTCGTGCCAGCTGCATTAATGAATCGGCCA |
| | | | ACGCGCGGGGAGAGGCGGTTTGCGTATTGGGCGCT |
| | | | CTTCCGCTTCCTCGCTCACTGACTCGCTGCGCTCGG |
| | | | TCGTTCGGCTGCGGCGAGCGGTATCAGCTCACTCA |
| | | | AAGGCGGTAATACGGTTATCCACAGAATCAGGGGA |
| | | | TAACGCAGGAAAGAACATGTGAGCAAAAGGCCAG |
| | | | CAAAAGGCCAGGAACCGTAAAAAGGCCGCGTTGCT |
| | | | GGCGTTTTTCCATAGGCTCCGCCCCCCTGACGAGCA |
| | | | TCACAAAAATCGACGCTCAAGTCAGAGGTGGCGAA |
| | | | ACCCGACAGGACTATAAAGATACCAGGCGTTTCCC |
| | | | CCTGGAAGCTCCCTCGTGCGCTCTCCTGTTCCGACC |
| | | | CTGCCGCTTACCGGATACCTGTCCGCCTTTCTCCCT |
| | | | TCGGGAAGCGTGGCGCTTTCTCATAGCTCACGCTGT |
| | | | AGGTATCTCAGTTCGGTGTAGGTCGTTCGCTCCAAG |
| | | | CTGGGCTGTGTGCACGAACCCCCCGTTCAGCCCGA |
| | | | CCGCTGCGCCTTATCCGGTAACTATCGTCTTGAGTC |
| | | | CAACCCGGTAAGACACGACTTATCGCCACTGGCAG |
| | | | CAGCCACTGGTAACAGGATTAGCAGAGCGAGGTAT |
| | | | GTAGGCGGTGCTACAGAGTTCTTGAAGTGGTGGCC |
| | | | TAACTACGGCTACACTAGAAGAACAGTATTTGGTA |
| | | | TCTGCGCTCTGCTGAAGCCAGTTACCTTCGGAAAA |
| | | | AGAGTTGGTAGCTCTTGATCCGGCAAACAAACCAC |
| | | | CGCTGGTAGCGGTGGTTTTTTTGTTTGCAAGCAGCA |
| | | | GATTACGCGCAGAAAAAAAGGATCTCAAGAAGAT |
| | | | CCTTTGATCTTTTCTACGGGGTCTGACGCTCAGTGG |
| | | | AACGAAAACTCACGTTAAGGGATTTTGGTCATGAG |
| | | | ATTATCAAAAAGGATCTTCACCTAGATCCTTTTAAA |
| | | | TTAAAAATGAAGTTTTAAATCAATCTAAAGTATAT |
| | | | ATGAGTAAACTTGGTCTGACAGTTAGAAAAACTCA |
| | | | TCGAGCATCAAATGAAACTGCAATTTATTCATATC |
| | | | AGGATTATCAATACCATATTTTTGAAAAAGCCGTTT |
| | | | CTGTAATGAAGGAGAAAACTCACCGAGGCAGTTCC |
| | | | ATAGGATGGCAAGATCCTGGTATCGGTCTGCGATT |
| | | | CCGACTCGTCCAACATCAATACAACCTATTAATTTC |
| | | | CCCTCGTCAAAAATAAGGTTATCAAGTGAGAAATC |
| | | | ACCATGAGTGACGACTGAATCCGGTGAGAATGGCA |
| | | | AAAGTTTATGCATTTCTTTCCAGACTTGTTCAACAG |
| | | | GCCAGCCATTACGCTCGTCATCAAAATCACTCGCA |
| | | | TCAACCAAACCGTTATTCATTCGTGATTGCGCCTGA |
| | | | GCGAGACGAAATACGCGATCGCTGTTAAAAGGACA |
| | | | ATTACAAACAGGAATCGAATGCAACCGGCGCAGG |
| | | | AACACTGCCAGCGCATCAACAATATTTTCACCTGA |
| | | | ATCAGGATATTCTTCTAATACCTGGAATGCTGTTTT |
| | | | CCCAGGGATCGCAGTGGTGAGTAACCATGCATCAT |
| | | | CAGGAGTACGGATAAAATGCTTGATGGTCGGAAGA |
| | | | GGCATAAATTCCGTCAGCCAGTTTAGTCTGACCATC |
| | | | TCATCTGTAACATCATTGGCAACGCTACCTTTGCCA |
| | | | TGTTTCAGAAACAACTCTGGCGCATCGGGCTTCCC |
| | | | ATACAATCGATAGATTGTCGCACCTGATTGCCCGA |
| | | | CATTATCGCGAGCCCATTTATACCCATATAAATCAG |
| | | | CATCCATGTTGGAATTTAATCGCGGCCTAGAGCAA |
| | | | GACGTTTCCCGTTGAATATGGCTCATACTCTTCCTT |
| | | | TTTCAATATTATTGAAGCATTTATCAGGGTTATTGT |
| | | | CTCATGAGCGGATACATATTTGAATGTATTTAGAA |
| | | | AAATAAACAAATAGGGGTTCCGCGCACATTTCCCC |
| | | | GAAAAGTGCCACCTGACGTCTAAGAAACCATTATT |
| | | | ATCATGACATTAACCTATAAAAATAGGCGTATCAC |
| | | | GAGGCCCTTTCGTCTCGCGCGTTTCGGTGATGACGG |
| | | | TGAAAACCTCTGACACATGCAGCTCCCGGAGACGG |
| | | | TCACAGCTTGTCTGTAAGCGGATGCCGGGAGCAGA |
| | | | CAAGCCCGTCAGGGCGCGTCAGCGGGTGTTGGCGG |
| | | | GTGTCGGGGCTGGCTTAACTATGCGGCATCAGAGC |
| | | | AGATTGTACTGAGAGTGCACCATATGCGGTGTGAA |
| | | | ATACCGCACAGATGCGTAAGGAGAAAATACCGCAT |
| | | | CAGGCGCCATTCGCCATTCAGGCTGCGCAACTGTT |
| | | | GGGAAGGGCGATCGGTGCGGGCCTCTTCGCTATTA |
| | | | CGCCAGCTGGCGAAAGGGGGATGTGCTGCAAGGC |
| | | | GATTAAGTTGGGTAACGCCAGGGTTTTCCCAGTCA |
| | | | CGACGTTGTAAAACGACGGCCAGTGAATTGACGCG |
| | | | TATTGGGATCGCGCAGCCGCC |
| 30 | pYZZ410 | Rep2.Cap9- | atgccggggttttacgagattgtgattaaggtccccagcgaccttgacgagcatct |
| | | p5.L4.p5 | gcccggcatttctgacagctttgtgaactgggtggccgagaaggaatgggagttg |
| | | | ccgccagattctgacatggatctgaatctgattgagcaggcacccctgaccgtggc |
| | | | cgagaagctgcagcgcgactttctgacggaatggcgccgtgtgagtaaggcccc |
| | | | ggaggctcttttctttgtgcaatttgagaagggagagagctacttccacatgcacgt |
| | | | gctcgtggaaaccaccggggtgaaatccatggttttgggacgtttcctgagtcaga |
| | | | ttcgcgaaaaactgattcagagaatttaccgcgggatcgagccgactttgccaaa |
| | | | ctggttcgcggtcacaaagaccagaaatggcgccggaggcgggaacaaggtg |
| | | | gtggatgagtgctacatccccaattacttgctccccaaaacccagcctgagctcca |
| | | | gtgggcgtggactaatatggaacagtatttaagcgcctgtttgaatctcacggagc |
| | | | gtaaacggttggtggcgcagcatctgacgcacgtgtcgcagacgcaggagcag |
| | | | aacaaagagaatcagaatcccaattctgatgcgccggtgatcagatcaaaaact |
| | | | tcagccaggtacatggagctggtcgggtggctcgtggacaaggggattacctcg |
| | | | gagaagcagtggatccaggaggaccaggcctcatacatctccttcaatgcggcct |
| | | | ccaactcgcggtcccaaatcaaggctgccttggacaatgcgggaaagattatga |
| | | | gcctgactaaaaccgcccccgactacctggtgggccagcagcccgtggaggac |
| | | | atttccagcaatcggatttataaaattttggaactaaacgggtacgatccccaatat |
| | | | gcggcttccgtctttctgggatgggccacgaaaaagttcggcaagaggaacacc |
| | | | atctggctgtttgggcctgcaactaccgggaagaccaacatcgcggaggccata |
| | | | gcccacactgtgcccttctacgggtgcgtaaactggaccaatgagaactttcccttc |
| | | | aacgactgtgtcgacaagatggtgatctggtgggaggaggggaagatgaccgc |
| | | | caaggtcgtggagtcggccaaagccattctcggaggaagcaaggtgcgcgtgg |
| | | | accagaaatgcaagtcctcggcccagatagacccgactcccgtgatcgtcacctc |
| | | | caacaccaacatgtgcgccgtgattgacgggaactcaacgaccttcgaacacca |
| | | | gcagccgttgcaagaccggatgttcaaatttgaactcacccgccgtctggatcatg |
| | | | actttgggaaggtcaccaagcaggaagtcaaagactttttccggtgggcaaagg |
| | | | atcacgtggttgaggtggagcatgaattctacgtcaaaaagggtggagccaaga |
| | | | aaagacccgcccccagtgacgcagatataagtgagcccaaacgggtgcgcga |
| | | | gtcagttgcgcagccatcgacgtcagacgcggaagcttcgatcaactacgcaga |
| | | | caggtaccaaaacaaatgttctcgtcacgtgggcatgaatctgatgtgtttccctg |
| | | | cagacaatgcgagagaatgaatcagaattcaaatatctgcttcactcacggaca |
| | | | gaaagactgtttagagtgctttcccgtgtcagaatctcaacccgtttctgtcgtcaaa |
| | | | aaggcgtatcagaaactgtgctacattcatcatatcatgggaaaggtgccagacg |
| | | | cttgcactgcctgcgatctggtcaatgtggatttggatgactgcatctttgaacaata |
| | | | aatgatttaaatcaggtatggctgccgatggttatcttccagattggctcgaggacaac |
| | | | cttagtgaaggaattcgcgagtggtgggctttgaaacctggagcccctcaacccaag |
| | | | gcaaatcaacaacatcaagacaacgctcgaggtcttgtgcttccgggttacaaatacc |
| | | | ttggacccggcaacggactcgacaagggggagccggtcaacgcagcagacgcg |
| | | | gcggccctcgagcacgacaaggcctacgaccagcagctcaaggccggagacaac |
| | | | ccgtacctcaagtacaaccacgccgacgccgagttccaggagcggctcaaagaag |
| | | | atacgtcttttgggggcaacctcgggcgagcagtcttccaggccaaaaagaggcttc |
| | | | ttgaacctcttggtctggttgaggaagcggctaagacggctcctggaaagaagagg |
| | | | cctgtagagcagtctcctcaggaaccggactcctccgcgggtattggcaaatcgggt |
| | | | gcacagcccgctaaaaagagactcaatttcggtcagactggcgacacagagtcagt |
| | | | cccagaccctcaaccaatcggagaacctcccgcagccccctcaggtgtgggatctc |
| | | | ttacaatggcttcaggtggtggcgcaccagtggcagacaataacgaaggtgccgat |
| | | | ggagtgggtagttcctcgggaaattggcattgcgattcccaatggctgggggacaga |
| | | | gtcatcaccaccagcacccgaacctgggccctgcccacctacaacaatcacctctac |
| | | | aagcaaatctccaacagcacatctggaggatcttcaaatgacaacgcctacttcggct |
| | | | acagcaccccctgggggtattttgacttcaacagattccactgccacttctcaccacgt |
| | | | gactggcagcgactcatcaacaacaactggggattccggcctaagcgactcaacttc |
| | | | aagctcttcaacattcaggtcaaagaggttacggacaacaatggagtcaagaccatc |
| | | | gccaataaccttaccagcacggtccaggtcttcacggactcagactatcagctcccgt |
| | | | acgtgctcgggtcggctcacgagggctgcctcccgccgttcccagcggacgttttca |
| | | | tgattcctcagtacgggtatctgacgcttaatgatggaagccaggccgtgggtcgttc |
| | | | gtccttttactgcctggaatatttcccgtcgcaaatgctaagaacgggtaacaacttcc |
| | | | agttcagctacgagtttgagaacgtacctttccatagcagctacgctcacagccaaag |
| | | | cctggaccgactaatgaatccactcatcgaccaatacttgtactatctctcaaagactat |
| | | | taacgcttctggacagaatcaacaaacgctaaaattcagtgtggccggacccagcaa |
| | | | catggctgtccagggaagaaactacatacctggacccagctaccgacaacaacgtg |
| | | | tctcaaccactgtgactcaaaacaacaacagcgaatttgcttggcctggagcttcttctt |
| | | | gggctctcaatggacgtaatagcttgatgaatcctggacctgctatggccagccaca |
| | | | aagaaggagaggaccgtttctttcctttgtctggatctttaatttttggcaaacaaggaa |
| | | | ctggaagagacaacgtggatgcggacaaagtcatgataaccaacgaagaagaaatt |
| | | | aaaactactaacccggtagcaacggagtcctatggacaagtggccacaaaccacca |
| | | | gagtgcccaagcacaggcgcagaccggctgggttcaaaaccaaggaatacttccg |
| | | | ggtatggtttggcaggacagagatgtgtacctgcaaggacccatttgggccaaaatt |
| | | | cctcacacggacggcaactttcacccttctccgctgatgggagggtttggaatgaag |
| | | | cacccgcctcctcagatcctcatcaaaaacacacctgtacctgcggatcctccaacg |
| | | | gccttcaacaaggacaagctgaactctttcatcacccagtattctactggccaagtca |
| | | | gcgtggagatcgagtgggagctgcagaaggaaaacagcaagcgctggaacccgg |
| | | | agatccagtacacttccaactattacaagtctaataatgttgaatttgctgttaatactga |
| | | | aggtgtatatagtgaaccccgccccattggcaccagatacctgactcgtaatctgtaa |
| | | | ttgcttgttaatcaataaaccgtttaattcgtttcagttgaACTTTGGTCTC |
| | | | TGCCCTAGGGGAGGTCCTGTATTAGAGGTCACGT |
| | | | GAGTGTTTTGCGACATTTTGCGACACCATGTGG |
| | | | TCACGCTGGGTATTTAAGCCCGAGTGAGCACGC |
| | | | AGGGTCTCCATTTTGAAGCGGGAGGTTTGAAGT |
| | | | CTTACCCCCAATAAGGGGATTTGCTCTATTTAATTAGGA |
| | | | ATAAGGTCGATTACTGATAGAACAAATCCAGGCTACTG |
| | | | TGTTTAGTAATCAGATTTGTTCGTGACCGATATGCACG |
| | | | GGCAAAACGGCAGGAGGTTGTTAGCGCGACCTCCTGC |
| | | | CACCCGCTTTCACGAAGGTCATGTGTAAAAGGCCGCA |
| | | | GCGTAACTAgaggtcctgtattagaggtcacgtgagtgttttgcgacatttt |
| | | | gcgacaccatgtggtcacgctgggtatttaagcccgagtgagcacgcagggtct |
| | | | ccattttgaagcgggaggtttgaaATCCCAATGGCGCGCCGAG |
| | | | CTTGGCTCGAGCATGGTCATAGCTGTTTCCTGTGTG |
| | | | AAATTGTTATCCGCTCACAATTCCACACAACATAC |
| | | | GAGCCGGAAGCATAAAGTGTAAAGCCTGGGGTGCC |
| | | | TAATGAGTGAGCTAACTCACATTAATTGCGTTGCG |
| | | | CTCACTGCCCGCTTTCCAGTCGGGAAACCTGTCGTG |
| | | | CCAGCTGCATTAATGAATCGGCCAACGCGCGGGGA |
| | | | GAGGCGGTTTGCGTATTGGGCGCTCTTCCGCTTCCT |
| | | | CGCTCACTGACTCGCTGCGCTCGGTCGTTCGGCTGC |
| | | | GGCGAGCGGTATCAGCTCACTCAAAGGCGGTAATA |
| | | | CGGTTATCCACAGAATCAGGGGATAACGCAGGAAA |
| | | | GAACATGTGAGCAAAAGGCCAGCAAAAGGCCAGG |
| | | | AACCGTAAAAAGGCCGCGTTGCTGGCGTTTTTCCA |
| | | | TAGGCTCCGCCCCCCTGACGAGCATCACAAAAATC |
| | | | GACGCTCAAGTCAGAGGTGGCGAAACCCGACAGG |
| | | | ACTATAAAGATACCAGGCGTTTCCCCCTGGAAGCT |
| | | | CCCTCGTGCGCTCTCCTGTTCCGACCCTGCCGCTTA |
| | | | CCGGATACCTGTCCGCCTTTCTCCCTTCGGGAAGCG |
| | | | TGGCGCTTTCTCATAGCTCACGCTGTAGGTATCTCA |
| | | | GTTCGGTGTAGGTCGTTCGCTCCAAGCTGGGCTGTG |
| | | | TGCACGAACCCCCCGTTCAGCCCGACCGCTGCGCC |
| | | | TTATCCGGTAACTATCGTCTTGAGTCCAACCCGGTA |
| | | | AGACACGACTTATCGCCACTGGCAGCAGCCACTGG |
| | | | TAACAGGATTAGCAGAGCGAGGTATGTAGGCGGTG |
| | | | CTACAGAGTTCTTGAAGTGGTGGCCTAACTACGGC |
| | | | TACACTAGAAGAACAGTATTTGGTATCTGCGCTCT |
| | | | GCTGAAGCCAGTTACCTTCGGAAAAAGAGTTGGTA |
| | | | GCTCTTGATCCGGCAAACAAACCACCGCTGGTAGC |
| | | | GGTGGTTTTTTTGTTTGCAAGCAGCAGATTACGCGC |
| | | | AGAAAAAAAGGATCTCAAGAAGATCCTTTGATCTT |
| | | | TTCTACGGGGTCTGACGCTCAGTGGAACGAAAACT |
| | | | CACGTTAAGGGATTTTGGTCATGAGATTATCAAAA |
| | | | AGGATCTTCACCTAGATCCTTTTAAATTAAAAATGA |
| | | | AGTTTTAAATCAATCTAAAGTATATATGAGTAAAC |
| | | | TTGGTCTGACAGTTAGAAAAACTCATCGAGCATCA |
| | | | AATGAAACTGCAATTTATTCATATCAGGATTATCA |
| | | | ATACCATATTTTTGAAAAAGCCGTTTCTGTAATGAA |
| | | | GGAGAAAACTCACCGAGGCAGTTCCATAGGATGGC |
| | | | AAGATCCTGGTATCGGTCTGCGATTCCGACTCGTCC |
| | | | AACATCAATACAACCTATTAATTTCCCCTCGTCAAA |
| | | | AATAAGGTTATCAAGTGAGAAATCACCATGAGTGA |
| | | | CGACTGAATCCGGTGAGAATGGCAAAAGTTTATGC |
| | | | ATTTCTTTCCAGACTTGTTCAACAGGCCAGCCATTA |
| | | | CGCTCGTCATCAAAATCACTCGCATCAACCAAACC |
| | | | GTTATTCATTCGTGATTGCGCCTGAGCGAGACGAA |
| | | | ATACGCGATCGCTGTTAAAAGGACAATTACAAACA |
| | | | GGAATCGAATGCAACCGGCGCAGGAACACTGCCA |
| | | | GCGCATCAACAATATTTTCACCTGAATCAGGATATT |
| | | | CTTCTAATACCTGGAATGCTGTTTTCCCAGGGATCG |
| | | | CAGTGGTGAGTAACCATGCATCATCAGGAGTACGG |
| | | | ATAAAATGCTTGATGGTCGGAAGAGGCATAAATTC |
| | | | CGTCAGCCAGTTTAGTCTGACCATCTCATCTGTAAC |
| | | | ATCATTGGCAACGCTACCTTTGCCATGTTTCAGAAA |
| | | | CAACTCTGGCGCATCGGGCTTCCCATACAATCGAT |
| | | | AGATTGTCGCACCTGATTGCCCGACATTATCGCGA |
| | | | GCCCATTTATACCCATATAAATCAGCATCCATGTTG |
| | | | GAATTTAATCGCGGCCTAGAGCAAGACGTTTCCCG |
| | | | TTGAATATGGCTCATACTCTTCCTTTTTCAATATTAT |
| | | | TGAAGCATTTATCAGGGTTATTGTCTCATGAGCGG |
| | | | ATACATATTTGAATGTATTTAGAAAAATAAACAAA |
| | | | TAGGGGTTCCGCGCACATTTCCCCGAAAAGTGCCA |
| | | | CCTGACGTCTAAGAAACCATTATTATCATGACATTA |
| | | | ACCTATAAAAATAGGCGTATCACGAGGCCCTTTCG |
| | | | TCTCGCGCGTTTCGGTGATGACGGTGAAAACCTCT |
| | | | GACACATGCAGCTCCCGGAGACGGTCACAGCTTGT |
| | | | CTGTAAGCGGATGCCGGGAGCAGACAAGCCCGTCA |
| | | | GGGCGCGTCAGCGGGTGTTGGCGGGTGTCGGGGCT |
| | | | GGCTTAACTATGCGGCATCAGAGCAGATTGTACTG |
| | | | AGAGTGCACCATATGCGGTGTGAAATACCGCACAG |
| | | | ATGCGTAAGGAGAAAATACCGCATCAGGCGCCATT |
| | | | CGCCATTCAGGCTGCGCAACTGTTGGGAAGGGCGA |
| | | | TCGGTGCGGGCCTCTTCGCTATTACGCCAGCTGGCG |
| | | | AAAGGGGGATGTGCTGCAAGGCGATTAAGTTGGGT |
| | | | AACGCCAGGGTTTTCCCAGTCACGACGTTGTAAAA |
| | | | CGACGGCCAGTGAATTGACGCGTATTGGGATCGCG |
| | | | CAGCCGCC |
| 31 | pYZZ411 | Rep2.Cap9- | atgccggggttttacgagattgtgattaaggtccccagcgaccttgacgagcatct |
| | | p5.L5.p5 | gcccggcatttctgacagctttgtgaactgggtggccgagaaggaatgggagttg |
| | | | ccgccagattctgacatggatctgaatctgattgagcaggcacccctgaccgtggc |
| | | | cgagaagctgcagcgcgactttctgacggaatggcgccgtgtgagtaaggcccc |
| | | | ggaggctcttttctttgtgcaatttgagaagggagagagctacttccacatgcacgt |
| | | | gctcgtggaaaccaccggggtgaaatccatggttttgggacgtttcctgagtcaga |
| | | | ttcgcgaaaaactgattcagagaatttaccggggatcgagccgactttgccaaa |
| | | | ctggttcgcggtcacaaagaccagaaatggcgccggaggcgggaacaaggtg |
| | | | gtggatgagtgctacatccccaattacttgctccccaaaacccagcctgagctcca |
| | | | gtgggcgtggactaatatggaacagtatttaagcgcctgtttgaatctcacggagc |
| | | | gtaaacggttggtggcgcagcatctgacgcacgtgtcgcagacgcaggagcag |
| | | | aacaaagagaatcagaatcccaattctgatgcgccggtgatcagatcaaaaact |
| | | | tcagccaggtacatggagctggtcgggtggctcgtggacaaggggattacctcg |
| | | | gagaagcagtggatccaggaggaccaggcctcatacatctccttcaatgcggcct |
| | | | ccaactcgcggtcccaaatcaaggctgccttggacaatgcgggaaagattatga |
| | | | gcctgactaaaaccgcccccgactacctggtgggccagcagcccgtggaggac |
| | | | atttccagcaatcggatttataaaattttggaactaaacgggtacgatccccaatat |
| | | | gcggcttccgtctttctgggatgggccacgaaaaagttcggcaagaggaacacc |
| | | | atctggctgtttgggcctgcaactaccgggaagaccaacatcgcggaggccata |
| | | | gcccacactgtgcccttctacgggtgcgtaaactggaccaatgagaactttcccttc |
| | | | aacgactgtgtcgacaagatggtgatctggtgggaggaggggaagatgaccgc |
| | | | caaggtcgtggagtcggccaaagccattctcggaggaagcaaggtgcgcgtgg |
| | | | accagaaatgcaagtcctcggcccagatagacccgactcccgtgatcgtcacctc |
| | | | caacaccaacatgtgcgccgtgattgacgggaactcaacgaccttcgaacacca |
| | | | gcagccgttgcaagaccggatgttcaaatttgaactcacccgccgtctggatcatg |
| | | | actttgggaaggtcaccaagcaggaagtcaaagactttttccggtgggcaaagg |
| | | | atcacgtggttgaggtggagcatgaattctacgtcaaaaagggtggagccaaga |
| | | | aaagacccgcccccagtgacgcagatataagtgagcccaaacgggtgcgcga |
| | | | gtcagttgcgcagccatcgacgtcagacgcggaagcttcgatcaactacgcaga |
| | | | caggtaccaaaacaaatgttctcgtcacgtgggcatgaatctgatgctgtttccctg |
| | | | cagacaatgcgagagaatgaatcagaattcaaatatctgcttcactcacggaca |
| | | | gaaagactgtttagagtgctttcccgtgtcagaatctcaacccgtttctgtcgtcaaa |
| | | | aaggcgtatcagaaactgtgctacattcatcatatcatgggaaaggtgccagacg |
| | | | cttgcactgcctgcgatctggtcaatgtggatttggatgactgcatctttgaacaata |
| | | | aatgatttaaatcaggtatggctgccgatggttatcttccagattggctcgaggacaac |
| | | | cttagtgaaggaattcgcgagtggtgggctttgaaacctggagcccctcaacccaag |
| | | | gcaaatcaacaacatcaagacaacgctcgaggtcttgtgcttccgggttacaaatacc |
| | | | ttggacccggcaacggactcgacaagggggagccggtcaacgcagcagacgcg |
| | | | gcggccctcgagcacgacaaggcctacgaccagcagctcaaggccggagacaac |
| | | | ccgtacctcaagtacaaccacgccgacgccgagttccaggagcggctcaaagaag |
| | | | atacgtcttttgggggcaacctcgggcgagcagtcttccaggccaaaaagaggcttc |
| | | | ttgaacctcttggtctggttgaggaagcggctaagacggctcctggaaagaagagg |
| | | | cctgtagagcagtctcctcaggaaccggactcctccgcgggtattggcaaatcgggt |
| | | | gcacagcccgctaaaaagagactcaatttcggtcagactggcgacacagagtcagt |
| | | | cccagaccctcaaccaatcggagaacctcccgcagccccctcaggtgtgggatctc |
| | | | ttacaatggcttcaggtggtggcgcaccagtcgcagacaataacgaaggtgccgat |
| | | | ggagtgggtagttcctcgggaaattggcattgcgattcccaatggctgggggacaga |
| | | | gtcatcaccaccagcacccgaacctgggccctgcccacctacaacaatcacctctac |
| | | | aagcaaatctccaacagcacatctggaggatcttcaaatgacaacgcctacttcggct |
| | | | acagcaccccctgggggtattttgacttcaacagattccactgccacttctcaccacgt |
| | | | gactggcagcgactcatcaacaacaactggggattccggcctaagcgactcaacttc |
| | | | aagctcttcaacattcaggtcaaagagcttacggacaacaatggagtcaagaccatc |
| | | | gccaataaccttaccagcacggtccaggtcttcacggactcagactatcagctcccgt |
| | | | acgtgctcgggtcggctcacgagggctgcctcccgccgttcccagcggacgttttca |
| | | | tgattcctcagtacgggtatctgacgcttaatgatggaagccaggccgtgggtcgttc |
| | | | gtccttttactgcctggaatatttcccgtcgcaaatgctaagaacgggtaacaacttcc |
| | | | agttcagctacgagtttgagaacgtacctttccatagcagctacgctcacagccaaag |
| | | | cctggaccgactaatgaatccactcatcgaccaatacttgtactatctctcaaagactat |
| | | | taacggttctggacagaatcaacaaacgctaaaattcagtgtggccggacccagcaa |
| | | | catggctctccagggaagaaactacatacctggacccagctaccgacaacaacgtg |
| | | | tctcaaccactgtgactcaaaacaacaacagcgaatttgcttggcctggagcttcttctt |
| | | | gggctctcaatggacgtaatagcttgatgaatcctggacctgctatggccagccaca |
| | | | aagaaggagaggaccgtttctttcctttgtctggatctttaatttttggcaaacaaggaa |
| | | | ctggaagagacaacgtggatgcggacaaagtcatgataaccaacgaagaagaaatt |
| | | | aaaactactaacccggtagcaacggagtcctatggacaagtggccacaaaccacca |
| | | | gagtgcccaagcacaggcgcagaccggctgggttcaaaaccaaggaatacttccg |
| | | | ggtatggtttggcaggacagagatgtgtacctgcaaggacccatttgggccaaaatt |
| | | | cctcacacggacggcaactttcacccttctccgctgatgggagggtttggaatgaag |
| | | | cacccgcctcctcagatcctcatcaaaaacacacctgtacctgcggatcctccaacg |
| | | | gccttcaacaaggacaagctgaactctttcatcacccagtattctactggccaagtca |
| | | | gcgtggagatcgagtgggagctgcagaaggaaaacagcaagcgctggaacccgg |
| | | | agatccagtacacttccaactattacaagtctaataatgttgaatttgctgttaatactga |
| | | | aggtgtatatagtgaaccccgccccattggcaccagatacctgactcgtaatctgtaa |
| | | | ttgcttgttaatcaataaaccgtttaattcgtttcagttgaACTTTGGTCTC |
| | | | TGCCCTAGGGGAGGTCCTGTATTAGAGGTCACGT |
| | | | GAGTGTTTTGCGACATTTTGCGACACCATGTGG |
| | | | TCACGCTGGGTATTTAAGCCCGAGTGAGCACGC |
| | | | AGGGTCTCCATTTTGAAGCGGGAGGTTTGAAGT |
| | | | CTTACCCCCAATAAGGGGATTTGCTCTATTTAATTAGGA |
| | | | ATAAGGTCGATTACTGATAGAACAAATCCAGGCTACTG |
| | | | TGTTTAGTAATCAGATTTGTTCGTGACCGATATGCACG |
| | | | GGCAAAACGGCAGGAGGTTGTTAGCGCGACCTCCTGC |
| | | | CACCCGCTTTCACGAAGGTCATGTGTAAAAGGCCGCA |
| | | | GCGTAACTATTACTAATGAATTCAGGACAGACAGTGGC |
| | | | TACGGCTCAGTTTGGGTTGTGCTGTTGCTGGGCGGCG |
| | | | ATGACGCCTGTACGCATTTGGTGATCCGGTTCTGgaggt |
| | | | cctgtattagaggtcacgtgagtgttttgcgacattttgcgacaccatgtggtcac |
| | | | gctgggtatttaagcccgagtgagcacgcagggtctccattttgaagcgggagg |
| | | | tttgaaATCCCAATGGCGCGCCGAGCTTGGCTCGAGC |
| | | | ATGGTCATAGCTGTTTCCTGTGTGAAATTGTTATCC |
| | | | GCTCACAATTCCACACAACATACGAGCCGGAAGCA |
| | | | TAAAGTGTAAAGCCTGGGGTGCCTAATGAGTGAGC |
| | | | TAACTCACATTAATTGCGTTGCGCTCACTGCCCGCT |
| | | | TTCCAGTCGGGAAACCTGTCGTGCCAGCTGCATTA |
| | | | ATGAATCGGCCAACGCGCGGGGAGAGGCGGTTTGC |
| | | | GTATTGGGCGCTCTTCCGCTTCCTCGCTCACTGACT |
| | | | CGCTGCGCTCGGTCGTTCGGCTGCGGCGAGCGGTA |
| | | | TCAGCTCACTCAAAGGCGGTAATACGGTTATCCAC |
| | | | AGAATCAGGGGATAACGCAGGAAAGAACATGTGA |
| | | | GCAAAAGGCCAGCAAAAGGCCAGGAACCGTAAAA |
| | | | AGGCCGCGTTGCTGGCGTTTTTCCATAGGCTCCGCC |
| | | | CCCCTGACGAGCATCACAAAAATCGACGCTCAAGT |
| | | | CAGAGGTGGCGAAACCCGACAGGACTATAAAGAT |
| | | | ACCAGGCGTTTCCCCCTGGAAGCTCCCTCGTGCGCT |
| | | | CTCCTGTTCCGACCCTGCCGCTTACCGGATACCTGT |
| | | | CCGCCTTTCTCCCTTCGGGAAGCGTGGCGCTTTCTC |
| | | | ATAGCTCACGCTGTAGGTATCTCAGTTCGGTGTAG |
| | | | GTCGTTCGCTCCAAGCTGGGCTGTGTGCACGAACC |
| | | | CCCCGTTCAGCCCGACCGCTGCGCCTTATCCGGTAA |
| | | | CTATCGTCTTGAGTCCAACCCGGTAAGACACGACT |
| | | | TATCGCCACTGGCAGCAGCCACTGGTAACAGGATT |
| | | | AGCAGAGCGAGGTATGTAGGCGGTGCTACAGAGTT |
| | | | CTTGAAGTGGTGGCCTAACTACGGCTACACTAGAA |
| | | | GAACAGTATTTGGTATCTGCGCTCTGCTGAAGCCA |
| | | | GTTACCTTCGGAAAAAGAGTTGGTAGCTCTTGATC |
| | | | CGGCAAACAAACCACCGCTGGTAGCGGTGGTTTTT |
| | | | TTGTTTGCAAGCAGCAGATTACGCGCAGAAAAAAA |
| | | | GGATCTCAAGAAGATCCTTTGATCTTTTCTACGGGG |
| | | | TCTGACGCTCAGTGGAACGAAAACTCACGTTAAGG |
| | | | GATTTTGGTCATGAGATTATCAAAAAGGATCTTCA |
| | | | CCTAGATCCTTTTAAATTAAAAATGAAGTTTTAAAT |
| | | | CAATCTAAAGTATATATGAGTAAACTTGGTCTGAC |
| | | | AGTTAGAAAAACTCATCGAGCATCAAATGAAACTG |
| | | | CAATTTATTCATATCAGGATTATCAATACCATATTT |
| | | | TTGAAAAAGCCGTTTCTGTAATGAAGGAGAAAACT |
| | | | CACCGAGGCAGTTCCATAGGATGGCAAGATCCTGG |
| | | | TATCGGTCTGCGATTCCGACTCGTCCAACATCAATA |
| | | | CAACCTATTAATTTCCCCTCGTCAAAAATAAGGTTA |
| | | | TCAAGTGAGAAATCACCATGAGTGACGACTGAATC |
| | | | CGGTGAGAATGGCAAAAGTTTATGCATTTCTTTCCA |
| | | | GACTTGTTCAACAGGCCAGCCATTACGCTCGTCATC |
| | | | AAAATCACTCGCATCAACCAAACCGTTATTCATTC |
| | | | GTGATTGCGCCTGAGCGAGACGAAATACGCGATCG |
| | | | CTGTTAAAAGGACAATTACAAACAGGAATCGAATG |
| | | | CAACCGGCGCAGGAACACTGCCAGCGCATCAACAA |
| | | | TATTTTCACCTGAATCAGGATATTCTTCTAATACCT |
| | | | GGAATGCTGTTTTCCCAGGGATCGCAGTGGTGAGT |
| | | | AACCATGCATCATCAGGAGTACGGATAAAATGCTT |
| | | | GATGGTCGGAAGAGGCATAAATTCCGTCAGCCAGT |
| | | | TTAGTCTGACCATCTCATCTGTAACATCATTGGCAA |
| | | | CGCTACCTTTGCCATGTTTCAGAAACAACTCTGGCG |
| | | | CATCGGGCTTCCCATACAATCGATAGATTGTCGCA |
| | | | CCTGATTGCCCGACATTATCGCGAGCCCATTTATAC |
| | | | CCATATAAATCAGCATCCATGTTGGAATTTAATCGC |
| | | | GGCCTAGAGCAAGACGTTTCCCGTTGAATATGGCT |
| | | | CATACTCTTCCTTTTTCAATATTATTGAAGCATTTAT |
| | | | CAGGGTTATTGTCTCATGAGCGGATACATATTTGA |
| | | | ATGTATTTAGAAAAATAAACAAATAGGGGTTCCGC |
| | | | GCACATTTCCCCGAAAAGTGCCACCTGACGTCTAA |
| | | | GAAACCATTATTATCATGACATTAACCTATAAAAA |
| | | | TAGGCGTATCACGAGGCCCTTTCGTCTCGCGCGTTT |
| | | | CGGTGATGACGGTGAAAACCTCTGACACATGCAGC |
| | | | TCCCGGAGACGGTCACAGCTTGTCTGTAAGCGGAT |
| | | | GCCGGGAGCAGACAAGCCCGTCAGGGCGCGTCAG |
| | | | CGGGTGTTGGCGGGTGTCGGGGCTGGCTTAACTAT |
| | | | GCGGCATCAGAGCAGATTGTACTGAGAGTGCACCA |
| | | | TATGCGGTGTGAAATACCGCACAGATGCGTAAGGA |
| | | | GAAAATACCGCATCAGGCGCCATTCGCCATTCAGG |
| | | | CTGCGCAACTGTTGGGAAGGGCGATCGGTGCGGGC |
| | | | CTCTTCGCTATTACGCCAGCTGGCGAAAGGGGGAT |
| | | | GTGCTGCAAGGCGATTAAGTTGGGTAACGCCAGGG |
| | | | TTTTCCCAGTCACGACGTTGTAAAACGACGGCCAG |
| | | | TGAATTGACGCGTATTGGGATCGCGCAGCCGCC |
| 32 | pYZZ412 | Rep2.Cap9- | atgccggggttttacgagattgtgattaaggtccccagcgaccttgacgagcatct |
| | | p5.L6.p5 | gcccggcatttctgacagctttgtgaactgggtggccgagaaggaatgggagttg |
| | | | ccgccagattctgacatggatctgaatctgattgagcaggcacccctgaccgtggc |
| | | | cgagaagctgcagcgcgactttctgacggaatggcgccgtgtgagtaaggcccc |
| | | | ggaggctcttttctttgtgcaatttgagaagggagagagctacttccacatgcacgt |
| | | | gctcgtggaaaccaccggggtgaaatccatggttttgggacgtttcctgagtcaga |
| | | | ttcgcgaaaaactgattcagagaatttaccgcgggatcgagccgactttgccaaa |
| | | | ctggttcgcggtcacaaagaccagaaatggcgccggaggcgggaacaaggtg |
| | | | gtggatgagtgctacatccccaattacttgctccccaaaacccagcctgagctcca |
| | | | gtgggcgtggactaatatggaacagtatttaagcgcctgtttgaatctcacggagc |
| | | | gtaaacggttggtggcgcagcatctgacgcacgtgtcgcagacgcaggagcag |
| | | | aacaaagagaatcagaatcccaattctgatgcgccggtgatcagatcaaaaact |
| | | | tcagccaggtacatggagctggtcgggtggctcgtggacaaggggattacctcg |
| | | | gagaagcagtggatccaggaggaccaggcctcatacatctccttcaatgcggcct |
| | | | ccaactcgcggtcccaaatcaaggctgccttggacaatgcgggaaagattatga |
| | | | gcctgactaaaaccgcccccgactacctggtgggccagcagcccgtggaggac |
| | | | atttccagcaatcggatttataaaattttggaactaaacgggtacgatccccaatat |
| | | | gcggcttccgtctttctgggatgggccacgaaaaagttcggcaagaggaacacc |
| | | | atctggctgtttgggcctgcaactaccgggaagaccaacatcgcggaggccata |
| | | | gcccacactgtgcccttctacgggtgcgtaaactggaccaatgagaactttcccttc |
| | | | aacgactgtgtcgacaagatggtgatctggtgggaggaggggaagatgaccgc |
| | | | caaggtcgtggagtcggccaaagccattctcggaggaagcaaggtgcgcgtgg |
| | | | accagaaatgcaagtcctcggcccagatagacccgactcccgtgatcgtcacctc |
| | | | caacaccaacatgtgcgccgtgattgacgggaactcaacgaccttcgaacacca |
| | | | gcagccgttgcaagaccggatgttcaaatttgaactcacccgccgtctggatcatg |
| | | | actttgggaaggtcaccaagcaggaagtcaaagactttttccggtgggcaaagg |
| | | | atcacgtggttgaggtggagcatgaattctacgtcaaaaagggtggagccaaga |
| | | | aaagacccgcccccagtgacgcagatataagtgagcccaaacgggtgcgcga |
| | | | gtcagttgcgcagccatcgacgtcagacgcggaagcttcgatcaactacgcaga |
| | | | caggtaccaaaacaaatgttctcgtcacgtgggcatgaatctgatgctgtttccctg |
| | | | cagacaatgcgagagaatgaatcagaattcaaatatctgcttcactcacggaca |
| | | | gaaagactgtttagagtgctttcccgtgtcagaatctcaacccgtttctgtcgtcaaa |
| | | | aaggcgtatcagaaactgtgctacattcatcatatcatgggaaaggtgccagacg |
| | | | cttgcactgcctgcgatctggtcaatgtggatttggatgactgcatctttgaacaata |
| | | | aatgatttaaatcaggtatggctgccgatggttatcttccagattggctcgaggacaac |
| | | | cttagtgaaggaattcgcgagtggtgggctttgaaacctggagcccctcaacccaag |
| | | | gcaaatcaacaacatcaagacaacgccgaggtcttgtgcttccgggttacaaatacc |
| | | | ttggacccggcaacggactcgacaagggggagccggtcaacgcagcagacgcg |
| | | | gcggccctcgagcacgacaaggcctacgaccagcagctcaaggccggagacaac |
| | | | ccgtacctcaagtacaaccacgccgacgccgagttccaggagcggctcaaagaag |
| | | | atacgtcttttgggggcaacctcgggcgagcagtcttccaggccaaaaagaggcttc |
| | | | ttgaacctcttggtctggttgaggaagcggctaagacggctcctggaaagaagagg |
| | | | cctgtagagcagtctcctcaggaaccggactcctccgcgggtattggcaaatcgggt |
| | | | gcacagcccgctaaaaagagactcaatttcggtcagactggcgacacagagtcagt |
| | | | cccagaccctcaaccaatcggagaacctcccgcagccccctcaggtgtgggatctc |
| | | | ttacaatggcttcaggtggtggcgcaccagtggcagacaataacgaaggtgccgat |
| | | | ggagtgggtagttcctcgggaaattggcattgcgattcccaatggctgggggacaga |
| | | | gtcatcaccaccagcacccgaacctgggccctgcccacctacaacaatcacctctac |
| | | | aagcaaatctccaacagcacatctggaggatcttcaaatgacaacgcctacttcggct |
| | | | acagcaccccctgggggtattttgacttcaacagattccactgccacttctcaccacgt |
| | | | gactggcagcgactcatcaacaacaactggggattccggcctaagcgactcaacttc |
| | | | aagctcttcaacattcaggtcaaagaggttacggacaacaatggagtcaagaccatc |
| | | | gccaataaccttaccagcacggtccaggtcttcacggactcagactatcagctcccgt |
| | | | acgtgctcgggtcggctcacgagggctgcctcccgccgttcccagcggacgttttca |
| | | | tgattcctcagtacgggtatctgacgcttaatgatggaagccaggccgtgggtcgttc |
| | | | gtccttttactgcctggaatatttcccgtcgcaaatgctaagaacgggtaacaacttcc |
| | | | agttcagctacgagtttgagaacgtacctttccatagcagctacgctcacagccaaag |
| | | | cctggaccgactaatgaatccactcatcgaccaatacttgtactatctctcaaagactat |
| | | | taacggttctggacagaatcaacaaacgctaaaattcagtgtggccggacccagcaa |
| | | | catggctgtccagggaagaaactacatacctggacccagctaccgacaacaacgtg |
| | | | tctcaaccactgtgactcaaaacaacaacagcgaatttgcttggcctggagcttcttctt |
| | | | gggctctcaatggacgtaatagcttgatgaatcctggacctgctatggccagccaca |
| | | | aagaaggagaggaccgtttctttcctttgtctggatctttaatttttggcaaacaaggaa |
| | | | ctggaagagacaacgtggatgcggacaaagtcatgataaccaacgaagaagaaatt |
| | | | aaaactactaacccggtagcaacggagtcctatggacaagtggccacaaaccacca |
| | | | gagtgcccaagcacaggcgcagaccggctgggttcaaaaccaaggaatacttccg |
| | | | ggtatggtttggcaggacagagatgtgtacctgcaaggacccatttgggccaaaatt |
| | | | cctcacacggacggcaactttcacccttctccgctgatgggagggtttggaatgaag |
| | | | cacccgcctcctcagatcctcatcaaaaacacacctgtacctgcggatcctccaacg |
| | | | gccttcaacaaggacaagctgaactctttcatcacccagtattctactggccaagtca |
| | | | gcgtggagatcgagtgggagctgcagaaggaaaacagcaagcgctggaacccgg |
| | | | agatccagtacacttccaactattacaagtctaataatgttgaatttgctgttaatactga |
| | | | aggtgtatatagtgaaccccgccccattggcaccagatacctgactcgtaatctgtaa |
| | | | ttgcttgttaatcaataaaccgtttaattcgtttcagttgaACTTTGGTCTC |
| | | | TGCCCTAGGGGAGGTCCTGTATTAGAGGTCACGT |
| | | | GAGTGTTTTGCGACATTTTGCGACACCATGTGG |
| | | | TCACGCTGGGTATTTAAGCCCGAGTGAGCACGC |
| | | | AGGGTCTCCATTTTGAAGCGGGAGGTTTGAACAT |
| | | | ACAGACGAAACTAGTTGGAGATTATCGAGCATTCTATC |
| | | | ACGTCGACGACCACTAGTGAGTTACTAGAGCCGAGGG |
| | | | GTAACGTTGATGCTCCTAAAAAgaggtcctgtattagaggtcacg |
| | | | tgagtgttttgcgacattttgcgacaccatgtggtcacgctgggtatttaagcccg |
| | | | agtgagcacgcagggtctccattttgaagcgggaggtttgaaATCCCAAT |
| | | | GGCGCGCCGAGCTTGGCTCGAGCATGGTCATAGCT |
| | | | GTTTCCTGTGTGAAATTGTTATCCGCTCACAATTCC |
| | | | ACACAACATACGAGCCGGAAGCATAAAGTGTAAA |
| | | | GCCTGGGGTGCCTAATGAGTGAGCTAACTCACATT |
| | | | AATTGCGTTGCGCTCACTGCCCGCTTTCCAGTCGGG |
| | | | AAACCTGTCGTGCCAGCTGCATTAATGAATCGGCC |
| | | | AACGCGCGGGGAGAGGCGGTTTGCGTATTGGGCGC |
| | | | TCTTCCGCTTCCTCGCTCACTGACTCGCTGCGCTCG |
| | | | GTCGTTCGGCTGCGGCGAGCGGTATCAGCTCACTC |
| | | | AAAGGCGGTAATACGGTTATCCACAGAATCAGGGG |
| | | | ATAACGCAGGAAAGAACATGTGAGCAAAAGGCCA |
| | | | GCAAAAGGCCAGGAACCGTAAAAAGGCCGCGTTG |
| | | | CTGGCGTTTTTCCATAGGCTCCGCCCCCCTGACGAG |
| | | | CATCACAAAAATCGACGCTCAAGTCAGAGGTGGCG |
| | | | AAACCCGACAGGACTATAAAGATACCAGGCGTTTC |
| | | | CCCCTGGAAGCTCCCTCGTGCGCTCTCCTGTTCCGA |
| | | | CCCTGCCGCTTACCGGATACCTGTCCGCCTTTCTCC |
| | | | CTTCGGGAAGCGTGGCGCTTTCTCATAGCTCACGCT |
| | | | GTAGGTATCTCAGTTCGGTGTAGGTCGTTCGCTCCA |
| | | | AGCTGGGCTGTGTGCACGAACCCCCCGTTCAGCCC |
| | | | GACCGCTGCGCCTTATCCGGTAACTATCGTCTTGAG |
| | | | TCCAACCCGGTAAGACACGACTTATCGCCACTGGC |
| | | | AGCAGCCACTGGTAACAGGATTAGCAGAGCGAGGT |
| | | | ATGTAGGCGGTGCTACAGAGTTCTTGAAGTGGTGG |
| | | | CCTAACTACGGCTACACTAGAAGAACAGTATTTGG |
| | | | TATCTGCGCTCTGCTGAAGCCAGTTACCTTCGGAAA |
| | | | AAGAGTTGGTAGCTCTTGATCCGGCAAACAAACCA |
| | | | CCGCTGGTAGCGGTGGTTTTTTTGTTTGCAAGCAGC |
| | | | AGATTACGCGCAGAAAAAAAGGATCTCAAGAAGA |
| | | | TCCTTTGATCTTTTCTACGGGGTCTGACGCTCAGTG |
| | | | GAACGAAAACTCACGTTAAGGGATTTTGGTCATGA |
| | | | GATTATCAAAAAGGATCTTCACCTAGATCCTTTTAA |
| | | | ATTAAAAATGAAGTTTTAAATCAATCTAAAGTATA |
| | | | TATGAGTAAACTTGGTCTGACAGTTAGAAAAACTC |
| | | | ATCGAGCATCAAATGAAACTGCAATTTATTCATAT |
| | | | CAGGATTATCAATACCATATTTTTGAAAAAGCCGTT |
| | | | TCTGTAATGAAGGAGAAAACTCACCGAGGCAGTTC |
| | | | CATAGGATGGCAAGATCCTGGTATCGGTCTGCGAT |
| | | | TCCGACTCGTCCAACATCAATACAACCTATTAATTT |
| | | | CCCCTCGTCAAAAATAAGGTTATCAAGTGAGAAAT |
| | | | CACCATGAGTGACGACTGAATCCGGTGAGAATGGC |
| | | | AAAAGTTTATGCATTTCTTTCCAGACTTGTTCAACA |
| | | | GGCCAGCCATTACGCTCGTCATCAAAATCACTCGC |
| | | | ATCAACCAAACCGTTATTCATTCGTGATTGCGCCTG |
| | | | AGCGAGACGAAATACGCGATCGCTGTTAAAAGGAC |
| | | | AATTACAAACAGGAATCGAATGCAACCGGCGCAG |
| | | | GAACACTGCCAGCGCATCAACAATATTTTCACCTG |
| | | | AATCAGGATATTCTTCTAATACCTGGAATGCTGTTT |
| | | | TCCCAGGGATCGCAGTGGTGAGTAACCATGCATCA |
| | | | TCAGGAGTACGGATAAAATGCTTGATGGTCGGAAG |
| | | | AGGCATAAATTCCGTCAGCCAGTTTAGTCTGACCA |
| | | | TCTCATCTGTAACATCATTGGCAACGCTACCTTTGC |
| | | | CATGTTTCAGAAACAACTCTGGCGCATCGGGCTTC |
| | | | CCATACAATCGATAGATTGTCGCACCTGATTGCCC |
| | | | GACATTATCGCGAGCCCATTTATACCCATATAAATC |
| | | | AGCATCCATGTTGGAATTTAATCGCGGCCTAGAGC |
| | | | AAGACGTTTCCCGTTGAATATGGCTCATACTCTTCC |
| | | | TTTTTCAATATTATTGAAGCATTTATCAGGGTTATT |
| | | | GTCTCATGAGCGGATACATATTTGAATGTATTTAGA |
| | | | AAAATAAACAAATAGGGGTTCCGCGCACATTTCCC |
| | | | CGAAAAGTGCCACCTGACGTCTAAGAAACCATTAT |
| | | | TATCATGACATTAACCTATAAAAATAGGCGTATCA |
| | | | CGAGGCCCTTTCGTCTCGCGCGTTTCGGTGATGACG |
| | | | GTGAAAACCTCTGACACATGCAGCTCCCGGAGACG |
| | | | GTCACAGCTTGTCTGTAAGCGGATGCCGGGAGCAG |
| | | | ACAAGCCCGTCAGGGCGCGTCAGCGGGTGTTGGCG |
| | | | GGTGTCGGGGCTGGCTTAACTATGCGGCATCAGAG |
| | | | CAGATTGTACTGAGAGTGCACCATATGCGGTGTGA |
| | | | AATACCGCACAGATGCGTAAGGAGAAAATACCGC |
| | | | ATCAGGCGCCATTCGCCATTCAGGCTGCGCAACTG |
| | | | TTGGGAAGGGCGATCGGTGCGGGCCTCTTCGCTAT |
| | | | TACGCCAGCTGGCGAAAGGGGGATGTGCTGCAAGG |
| | | | CGATTAAGTTGGGTAACGCCAGGGTTTTCCCAGTC |
| | | | ACGACGTTGTAAAACGACGGCCAGTGAATTGACGC |
| | | | GTATTGGGATCGCGCAGCCGCC |
| 33 | pYZZ413 | Rep2.Cap9- | atgccggggttttacgagattgtgattaaggtccccagcgaccttgacgagcatct |
| | | p5.L7.p5 | gcccggcatttctgacagctttgtgaactgggtggccgagaaggaatgggagttg |
| | | | ccgccagattctgacatggatctgaatctgattgagcaggcacccctgaccgtggc |
| | | | cgagaagctgcagcgcgactttctgacggaatggcgccgtgtgagtaaggcccc |
| | | | ggaggctcttttctttgtgcaatttgagaagggagagagctacttccacatgcacgt |
| | | | gctcgtggaaaccaccggggtgaaatccatggttttgggacgtttcctgagtcaga |
| | | | ttcgcgaaaaactgattcagagaatttaccgcgggatcgagccgactttgccaaa |
| | | | ctggttcgcggtcacaaagaccagaaatggcgccggaggcgggaacaaggtg |
| | | | gtggatgagtgctacatccccaattacttgctccccaaaacccagcctgagctcca |
| | | | gtgggcgtggactaatatggaacagtatttaagcgcctgtttgaatctcacggagc |
| | | | gtaaacggttggtggcgcagcatctgacgcacgtgtcgcagacgcaggagcag |
| | | | aacaaagagaatcagaatcccaattctgatgcgccggtgatcagatcaaaaact |
| | | | tcagccaggtacatggagctggtcgggtggctcgtggacaaggggattacctcg |
| | | | gagaagcagtggatccaggaggaccaggcctcatacatctccttcaatgcggcct |
| | | | ccaactcgcggtcccaaatcaaggctgccttggacaatgcgggaaagattatga |
| | | | gcctgactaaaaccgcccccgactacctggtgggccagcagcccgtggaggac |
| | | | atttccagcaatcggatttataaaattttggaactaaacgggtacgatccccaatat |
| | | | gcggcttccgtctttctgggatgggccacgaaaaagttcggcaagaggaacacc |
| | | | atctggctgtttgggcctgcaactaccgggaagaccaacatcgcggaggccata |
| | | | gcccacactgtgcccttctacgggtgcgtaaactggaccaatgagaactttcccttc |
| | | | aacgactgtgtcgacaagatggtgatctggtgggaggaggggaagatgaccgc |
| | | | caaggtcgtggagtcggccaaagccattctcggaggaagcaaggtgcgcgtgg |
| | | | accagaaatgcaagtcctcggcccagatagacccgactcccgtgatcgtcacctc |
| | | | caacaccaacatgtgcgccgtgattgacgggaactcaacgaccttcgaacacca |
| | | | gcagccgttgcaagaccggatgttcaaatttgaactcacccgccgtctggatcatg |
| | | | actttgggaaggtcaccaagcaggaagtcaaagactttttccggtgggcaaagg |
| | | | atcacgtggttgaggtggagcatgaattctacgtcaaaaagggtggagccaaga |
| | | | aaagacccgcccccagtgacgcagatataagtgagcccaaacgggtgcgcga |
| | | | gtcagttgcgcagccatcgacgtcagacgcggaagcttcgatcaactacgcaga |
| | | | caggtaccaaaacaaatgttctcgtcacgtgggcatgaatctgatgctgtttccctg |
| | | | cagacaatgcgagagaatgaatcagaattcaaatatctgcttcactcacggaca |
| | | | gaaagactgtttagagtgctttcccgtgtcagaatctcaacccgtttctgtcgtcaaa |
| | | | aaggcgtatcagaaactgtgctacattcatcatatcatgggaaaggtgccagacg |
| | | | cttgcactgcctgcgatctggtcaatgtggatttggatgactgcatctttgaacaata |
| | | | aatgatttaaatcaggtatggctgccgatggttatcttccagattggctcgaggacaac |
| | | | cttagtgaaggaattcgcgagtggtgggctttgaaacctggagcccctcaacccaag |
| | | | gcaaatcaacaacatcaagacaacgctcgaggtcttgtgcttccgggttacaaatacc |
| | | | ttggacccggcaacggactcgacaagggggagccggtcaacgcagcagacgcg |
| | | | gcggccctcgagcacgacaaggcctacgaccagcagctcaaggccggagacaac |
| | | | ccgtacctcaagtacaaccacgccgacgccgagttccaggagcggctcaaagaag |
| | | | atacgtcttttgggggcaacctcgggcgagcagtcttccaggccaaaaagaggcttc |
| | | | ttgaacctcttggtctggttgaggaagcggctaagacggctcctggaaagaagagg |
| | | | cctgtagagcagtctcctcaggaaccggactcctccgcgggtattggcaaatcgggt |
| | | | gcacagcccgctaaaaagagactcaatttcggtcagactggcgacacagagtcagt |
| | | | cccagaccctcaaccaatcggagaacctcccgcagccccctcaggtgtgggatctc |
| | | | ttacaatggcttcaggtggtggcgcaccagtggcagacaataacgaaggtgccgat |
| | | | ggagtgggtagttcctcgggaaattggcattgcgattcccaatggctgggggacaga |
| | | | gtcatcaccaccagcacccgaacctgggccctgcccacctacaacaatcacctctac |
| | | | aagcaaatctccaacagcacatctggaggatcttcaaatgacaacgcctacttcggct |
| | | | acagcaccccctgggggtattttgacttcaacagattccactgccacttctcaccacgt |
| | | | gactggcagcgactcatcaacaacaactggggattccggcctaagcgactcaacttc |
| | | | aagctcttcaacattcaggtcaaagagcttacggacaacaatggagtcaagaccatc |
| | | | gccaataaccttaccagcacggtccaggtcttcacggactcagactatcagctcccgt |
| | | | acgtgctcgggtcggctcacgagggctgcctcccgccgttcccagcggacgttttca |
| | | | tgattcctcagtacgggtatctgacgcttaatgatggaagccaggccgtgggtcgttc |
| | | | gtccttttactgcctggaatatttcccgtcgcaaatgctaagaacgggtaacaacttcc |
| | | | agttcagctacgagtttgagaacgtacctttccatagcagctacgctcacagccaaag |
| | | | cctggaccgactaatgaatccactcatcgaccaatacttgtactatctctcaaagactat |
| | | | taacggttctggacagaatcaacaaacgctaaaattcagtgtggccggacccagcaa |
| | | | catggctgtccagggaagaaactacatacctggacccagctaccgacaacaacgtg |
| | | | tctcaaccactgtgactcaaaacaacaacagcgaatttgcttggcctggagcttcttctt |
| | | | gggctctcaatggacgtaatagcttgatgaatcctggacctgctatggccagccaca |
| | | | aagaaggagaggaccgtttctttcctttctctggatctttaatttttggcaaacaaggaa |
| | | | ctggaagagacaacgtggatgcggacaaagtcatgataaccaacgaagaagaaatt |
| | | | aaaactactaacccggtagcaacggagtcctatggacaagtggccacaaaccacca |
| | | | gagtgcccaagcacaggcgcagaccggctgggttcaaaaccaaggaatacttccg |
| | | | ggtatggtttcgcaggacagagatgtgtacctgcaaggacccatttgggccaaaatt |
| | | | cctcacacggacggcaactttcacccttctccgctgatgggagggtttggaatgaag |
| | | | cacccgcctcctcagatcctcatcaaaaacacacctgtacctgcggatcctccaacg |
| | | | gccttcaacaaggacaagctgaactctttcatcacccagtattctactggccaagtca |
| | | | gcgtggagatcgagtgggagctgcagaaggaaaacagcaagcgctggaacccgg |
| | | | agatccagtacacttccaactattacaagtctaataatgttgaatttgctgttaatactga |
| | | | aggtgtatatagtgaaccccgccccattggcaccagatacctgactcgtaatctgtaa |
| | | | ttgcttgttaatcaataaaccgtttaattcgtttcagttgaACTTTGGTCTC |
| | | | TGCCCTAGGGGAGGTCCTGTATTAGAGGTCACGT |
| | | | GAGTGTTTTGCGACATTTTGCGACACCATGTGG |
| | | | TCACGCTGGGTATTTAAGCCCGAGTGAGCACGC |
| | | | AGGGTCTCCATTTTGAAGCGGGAGGTTTGAACAT |
| | | | ACAGACGAAACTAGTTGGAGATTATCGAGCATTCTATC |
| | | | ACGTCGACGACCACTAGTGAGTTACTAGAGCCGAGGG |
| | | | GTAACGTTGATGCTCCTAAAAATCTCTCGGTCGATGCA |
| | | | AGCGATTACACTCCTGTCACATCATAATCGTTTGCTATT |
| | | | CAGGGCTTGATCAACACTGGATTGCTTTTCACTTAAAG |
| | | | TATTATGgaggtcctgtattagaggtcacgtgagtgttttgcgacattttgcg |
| | | | acaccatgtggtcacgctgggtatttaagcccgagtgagcacgcagggtctcca |
| | | | ttttgaagcgggaggtttgaaATCCCAATGGCGCGCCGAGCTT |
| | | | GGCTCGAGCATGGTCATAGCTGTTTCCTGTGTGAA |
| | | | ATTGTTATCCGCTCACAATTCCACACAACATACGA |
| | | | GCCGGAAGCATAAAGTGTAAAGCCTGGGGTGCCTA |
| | | | ATGAGTGAGCTAACTCACATTAATTGCGTTGCGCTC |
| | | | ACTGCCCGCTTTCCAGTCGGGAAACCTGTCGTGCC |
| | | | AGCTGCATTAATGAATCGGCCAACGCGCGGGGAGA |
| | | | GGCGGTTTGCGTATTGGGCGCTCTTCCGCTTCCTCG |
| | | | CTCACTGACTCGCTGCGCTCGGTCGTTCGGCTGCGG |
| | | | CGAGCGGTATCAGCTCACTCAAAGGCGGTAATACG |
| | | | GTTATCCACAGAATCAGGGGATAACGCAGGAAAG |
| | | | AACATGTGAGCAAAAGGCCAGCAAAAGGCCAGGA |
| | | | ACCGTAAAAAGGCCGCGTTGCTGGCGTTTTTCCAT |
| | | | AGGCTCCGCCCCCCTGACGAGCATCACAAAAATCG |
| | | | ACGCTCAAGTCAGAGGTGGCGAAACCCGACAGGA |
| | | | CTATAAAGATACCAGGCGTTTCCCCCTGGAAGCTC |
| | | | CCTCGTGCGCTCTCCTGTTCCGACCCTGCCGCTTAC |
| | | | CGGATACCTGTCCGCCTTTCTCCCTTCGGGAAGCGT |
| | | | GGCGCTTTCTCATAGCTCACGCTGTAGGTATCTCAG |
| | | | TTCGGTGTAGGTCGTTCGCTCCAAGCTGGGCTGTGT |
| | | | GCACGAACCCCCCGTTCAGCCCGACCGCTGCGCCT |
| | | | TATCCGGTAACTATCGTCTTGAGTCCAACCCGGTAA |
| | | | GACACGACTTATCGCCACTGGCAGCAGCCACTGGT |
| | | | AACAGGATTAGCAGAGCGAGGTATGTAGGCGGTGC |
| | | | TACAGAGTTCTTGAAGTGGTGGCCTAACTACGGCT |
| | | | ACACTAGAAGAACAGTATTTGGTATCTGCGCTCTG |
| | | | CTGAAGCCAGTTACCTTCGGAAAAAGAGTTGGTAG |
| | | | CTCTTGATCCGGCAAACAAACCACCGCTGGTAGCG |
| | | | GTGGTTTTTTTGTTTGCAAGCAGCAGATTACGCGCA |
| | | | GAAAAAAAGGATCTCAAGAAGATCCTTTGATCTTT |
| | | | TCTACGGGGTCTGACGCTCAGTGGAACGAAAACTC |
| | | | ACGTTAAGGGATTTTGGTCATGAGATTATCAAAAA |
| | | | GGATCTTCACCTAGATCCTTTTAAATTAAAAATGAA |
| | | | GTTTTAAATCAATCTAAAGTATATATGAGTAAACTT |
| | | | GGTCTGACAGTTAGAAAAACTCATCGAGCATCAAA |
| | | | TGAAACTGCAATTTATTCATATCAGGATTATCAATA |
| | | | CCATATTTTTGAAAAAGCCGTTTCTGTAATGAAGG |
| | | | AGAAAACTCACCGAGGCAGTTCCATAGGATGGCAA |
| | | | GATCCTGGTATCGGTCTGCGATTCCGACTCGTCCAA |
| | | | CATCAATACAACCTATTAATTTCCCCTCGTCAAAAA |
| | | | TAAGGTTATCAAGTGAGAAATCACCATGAGTGACG |
| | | | ACTGAATCCGGTGAGAATGGCAAAAGTTTATGCAT |
| | | | TTCTTTCCAGACTTGTTCAACAGGCCAGCCATTACG |
| | | | CTCGTCATCAAAATCACTCGCATCAACCAAACCGT |
| | | | TATTCATTCGTGATTGCGCCTGAGCGAGACGAAAT |
| | | | ACGCGATCGCTGTTAAAAGGACAATTACAAACAGG |
| | | | AATCGAATGCAACCGGCGCAGGAACACTGCCAGCG |
| | | | CATCAACAATATTTTCACCTGAATCAGGATATTCTT |
| | | | CTAATACCTGGAATGCTGTTTTCCCAGGGATCGCA |
| | | | GTGGTGAGTAACCATGCATCATCAGGAGTACGGAT |
| | | | AAAATGCTTGATGGTCGGAAGAGGCATAAATTCCG |
| | | | TCAGCCAGTTTAGTCTGACCATCTCATCTGTAACAT |
| | | | CATTGGCAACGCTACCTTTGCCATGTTTCAGAAACA |
| | | | ACTCTGGCGCATCGGGCTTCCCATACAATCGATAG |
| | | | ATTGTCGCACCTGATTGCCCGACATTATCGCGAGCC |
| | | | CATTTATACCCATATAAATCAGCATCCATGTTGGAA |
| | | | TTTAATCGCGGCCTAGAGCAAGACGTTTCCCGTTG |
| | | | AATATGGCTCATACTCTTCCTTTTTCAATATTATTG |
| | | | AAGCATTTATCAGGGTTATTGTCTCATGAGCGGAT |
| | | | ACATATTTGAATGTATTTAGAAAAATAAACAAATA |
| | | | GGGGTTCCGCGCACATTTCCCCGAAAAGTGCCACC |
| | | | TGACGTCTAAGAAACCATTATTATCATGACATTAA |
| | | | CCTATAAAAATAGGCGTATCACGAGGCCCTTTCGT |
| | | | CTCGCGCGTTTCGGTGATGACGGTGAAAACCTCTG |
| | | | ACACATGCAGCTCCCGGAGACGGTCACAGCTTGTC |
| | | | TGTAAGCGGATGCCGGGAGCAGACAAGCCCGTCAG |
| | | | GGCGCGTCAGCGGGTGTTGGCGGGTGTCGGGGCTG |
| | | | GCTTAACTATGCGGCATCAGAGCAGATTGTACTGA |
| | | | GAGTGCACCATATGCGGTGTGAAATACCGCACAGA |
| | | | TGCGTAAGGAGAAAATACCGCATCAGGCGCCATTC |
| | | | GCCATTCAGGCTGCGCAACTGTTGGGAAGGGCGAT |
| | | | CGGTGCGGGCCTCTTCGCTATTACGCCAGCTGGCG |
| | | | AAAGGGGGATGTGCTGCAAGGCGATTAAGTTGGGT |
| | | | AACGCCAGGGTTTTCCCAGTCACGACGTTGTAAAA |
| | | | CGACGGCCAGTGAATTGACGCGTATTGGGATCGCG |
| | | | CAGCCGCC |
| 34 | pYZZ414 | Rep2.Cap9- | atgccggggttttacgagattgtgattaaggtccccagcgaccttgacgagcatct |
| | | p5.L8.p5 | gcccggcatttctgacagctttgtgaactgggtggccgagaaggaatgggagttg |
| | | | ccgccagattctgacatggatctgaatctgattgagcaggcacccctgaccgtggc |
| | | | cgagaagctgcagcgcgactttctgacggaatggcgccgtgtgagtaaggcccc |
| | | | ggaggctcttttctttgtgcaatttgagaagggagagagctacttccacatgcacgt |
| | | | gctcgtggaaaccaccggggtgaaatccatggttttgggacgtttcctgagtcaga |
| | | | ttcgcgaaaaactgattcagagaatttaccgcgggatcgagccgactttgccaaa |
| | | | ctggttcgcggtcacaaagaccagaaatggcgccggaggcgggaacaaggtg |
| | | | gtggatgagtgctacatccccaattacttgctccccaaaacccagcctgagctcca |
| | | | gtgggcgtggactaatatggaacagtatttaagcgcctgtttgaatctcacggagc |
| | | | gtaaacggttggtggcgcagcatctgacgcacgtgtcgcagacgcaggagcag |
| | | | aacaaagagaatcagaatcccaattctgatgcgccggtgatcagatcaaaaact |
| | | | tcagccaggtacatggagctggtcgggtggctcgtggacaaggggattacctcg |
| | | | gagaagcagtggatccaggaggaccaggcctcatacatctccttcaatgcggcct |
| | | | ccaactcgcggtcccaaatcaaggctgccttggacaatgcgggaaagattatga |
| | | | gcctgactaaaaccgcccccgactacctggtgggccagcagcccgtggaggac |
| | | | atttccagcaatcggatttataaaattttggaactaaacgggtacgatccccaatat |
| | | | gcggcttccgtctttctgggatgggccacgaaaaagttcggcaagaggaacacc |
| | | | atctggctgtttgggcctgcaactaccgggaagaccaacatcgcggaggccata |
| | | | gcccacactgtgcccttctacgggtgcgtaaactggaccaatgagaactttcccttc |
| | | | aacgactgtgtcgacaagatggtgatctggtgggaggaggggaagatgaccgc |
| | | | caaggtcgtggagtcggccaaagccattctcggaggaagcaaggtgcgcgtgg |
| | | | accagaaatgcaagtcctcggcccagatagacccgactcccgtgatcgtcacctc |
| | | | caacaccaacatgtgcgccgtgattgacgggaactcaacgaccttcgaacacca |
| | | | gcagccgttgcaagaccggatgttcaaatttgaactcacccgccgtctggatcatg |
| | | | actttgggaaggtcaccaagcaggaagtcaaagactttttccggtgggcaaagg |
| | | | atcacgtggttgaggtggagcatgaattctacgtcaaaaagggtggagccaaga |
| | | | aaagacccgcccccagtgacgcagatataagtgagcccaaacgggtgcgcga |
| | | | gtcagttgcgcagccatcgacgtcagacgcggaagcttcgatcaactacgcaga |
| | | | caggtaccaaaacaaatgttctcgtcacgtgggcatgaatctgatgctgtttccctg |
| | | | cagacaatgcgagagaatgaatcagaattcaaatatctgcttcactcacggaca |
| | | | gaaagactgtttagagtgctttcccgtgtcagaatctcaacccgtttctgtcgtcaaa |
| | | | aaggcgtatcagaaactgtgctacattcatcatatcatgggaaaggtgccagacg |
| | | | cttgcactgcctgcgatctggtcaatgtggatttggatgactgcatctttgaacaata |
| | | | aatgatttaaatcaggtatggctgccgatggttatcttccagattggctcgaggacaac |
| | | | cttagtgaaggaattcgcgagtggtgggctttgaaacctggagcccctcaacccaag |
| | | | gcaaatcaacaacatcaagacaacgctcgaggtcttgtgcttccgggttacaaatacc |
| | | | ttggacccggcaacggactcgacaagggggagccggtcaacgcagcagacgcg |
| | | | gcggccctcgagcacgacaaggcctacgaccagcagctcaaggccggagacaac |
| | | | ccgtacctcaagtacaaccacgccgacgccgagttccaggagcggctcaaagaag |
| | | | atacgtcttttgggggcaacctcgggcgagcagtcttccaggccaaaaagaggcttc |
| | | | ttgaacctcttggtctcgttgaggaagcggctaagacggctcctggaaagaagagg |
| | | | cctgtagagcagtctcctcaggaaccggactcctccgcgggtattggcaaatcgggt |
| | | | gcacagcccgctaaaaagagactcaatttcggtcagactggcgacacagagtcagt |
| | | | cccagaccctcaaccaatcggagaacctcccgcagccccctcaggtgtgggatctc |
| | | | ttacaatggcttcaggtggtggcgcaccagtggcagacaataacgaaggtgccgat |
| | | | ggagtgggtagttcctcgggaaattggcattgcgattcccaatggctgggggacaga |
| | | | gtcatcaccaccagcacccgaacctgggccctgcccacctacaacaatcacctctac |
| | | | aagcaaatctccaacagcacatctggaggatcttcaaatgacaacgcctacttcggct |
| | | | acagcaccccctgggggtattttgacttcaacagattccactgccacttctcaccacgt |
| | | | gactggcagcgactcatcaacaacaactggggattccggcctaagcgactcaacttc |
| | | | aagctcttcaacattcaggtcaaagaggttacggacaacaatggagtcaagaccatc |
| | | | gccaataaccttaccagcacggtccaggtcttcacggactcagactatcagctcccgt |
| | | | acgtgctcgggtcggctcacgagggctgcctcccgccgttcccagcggacgttttca |
| | | | tgattcctcagtacgggtatctgacgcttaatgatggaagccaggccgtgggtcgttc |
| | | | gtccttttactgcctggaatatttcccgtcgcaaatgctaagaacgggtaacaacttcc |
| | | | agttcagctacgagtttgagaacgtacctttccatagcagctacgctcacagccaaag |
| | | | cctggaccgactaatgaatccactcatcgaccaatacttgtactatctctcaaagactat |
| | | | taacggttctggacagaatcaacaaacgctaaaattcagtgtggccggacccagcaa |
| | | | catggctgtccagggaagaaactacatacctggacccagctaccgacaacaacgtg |
| | | | tctcaaccactgtgactcaaaacaacaacagcgaatttgcttggcctggagcttcttctt |
| | | | gggctctcaatggacgtaatagcttgatgaatcctggacctgctatggccagccaca |
| | | | aagaaggagaggaccgtttctttcctttctctggatctttaatttttcgcaaacaaggaa |
| | | | ctggaagagacaacgtggatgcggacaaagtcatgataaccaacgaagaagaaatt |
| | | | aaaactactaacccggtagcaacggagtcctatggacaagtggccacaaaccacca |
| | | | gagtgcccaagcacaggcgcagaccggctgggttcaaaaccaaggaatacttccg |
| | | | ggtatggtttggcaggacagagatgtgtacctgcaaggacccatttgggccaaaatt |
| | | | cctcacacggacggcaactttcacccttctccgctgatgggagggtttggaatgaag |
| | | | cacccgcctcctcagatcctcatcaaaaacacacctgtacctgcggatcctccaacg |
| | | | gccttcaacaaggacaagctgaactctttcatcacccagtattctactggccaagtca |
| | | | gcgtggagatcgagtgggagctgcagaaggaaaacagcaagcgctggaacccgg |
| | | | agatccagtacacttccaactattacaagtctaataatcttgaatttgctgttaatactga |
| | | | aggtgtatatagtgaaccccgccccattggcaccagatacctgactcgtaatctgtaa |
| | | | ttgcttgttaatcaataaaccgtttaattcgtttcagttgaACTTTGGTCTC |
| | | | TGCCCTAGGGGAGGTCCTGTATTAGAGGTCACGT |
| | | | GAGTGTTTTGCGACATTTTGCGACACCATGTGG |
| | | | TCACGCTGGGTATTTAAGCCCGAGTGAGCACGC |
| | | | AGGGTCTCCATTTTGAAGCGGGAGGTTTGAACAT |
| | | | ACAGACGAAACTAGTTGGAGATTATCGAGCATTCTATC |
| | | | ACGTCGACGACCACTAGTGAGTTACTAGAGCCGAGGG |
| | | | GTAACGTTGATGCTCCTAAAAATCTCTCGGTCGATGCA |
| | | | AGCGATTACACTCCTGTCACATCATAATCGTTTGCTATT |
| | | | CAGGGCTTGATCAACACTGGATTGCTTTTCACTTAAAG |
| | | | TATTATGCACGACAGGGTACGTGTACCATATAAACCTG |
| | | | TTATAACTTACCTCAGACTAGTTGGAAATGTGGCTAGAT |
| | | | CTTAGCTTACGTTACTAAAGGGTCCACGTTgaggtcctgtat |
| | | | tagaggtcacgtgagtgttttgcgacattttgcgacaccatgtggtcacgctggg |
| | | | tatttaagcccgagtgagcacgcagggtctccattttgaagcgggaggtttgaa |
| | | | ATCCCAATGGCGCGCCGAGCTTGGCTCGAGCATGG |
| | | | TCATAGCTGTTTCCTGTGTGAAATTGTTATCCGCTC |
| | | | ACAATTCCACACAACATACGAGCCGGAAGCATAAA |
| | | | GTGTAAAGCCTGGGGTGCCTAATGAGTGAGCTAAC |
| | | | TCACATTAATTGCGTTGCGCTCACTGCCCGCTTTCC |
| | | | AGTCGGGAAACCTGTCGTGCCAGCTGCATTAATGA |
| | | | ATCGGCCAACGCGCGGGGAGAGGCGGTTTGCGTAT |
| | | | TGGGCGCTCTTCCGCTTCCTCGCTCACTGACTCGCT |
| | | | GCGCTCGGTCGTTCGGCTGCGGCGAGCGGTATCAG |
| | | | CTCACTCAAAGGCGGTAATACGGTTATCCACAGAA |
| | | | TCAGGGGATAACGCAGGAAAGAACATGTGAGCAA |
| | | | AAGGCCAGCAAAAGGCCAGGAACCGTAAAAAGGC |
| | | | CGCGTTGCTGGCGTTTTTCCATAGGCTCCGCCCCCC |
| | | | TGACGAGCATCACAAAAATCGACGCTCAAGTCAGA |
| | | | GGTGGCGAAACCCGACAGGACTATAAAGATACCA |
| | | | GGCGTTTCCCCCTGGAAGCTCCCTCGTGCGCTCTCC |
| | | | TGTTCCGACCCTGCCGCTTACCGGATACCTGTCCGC |
| | | | CTTTCTCCCTTCGGGAAGCGTGGCGCTTTCTCATAG |
| | | | CTCACGCTGTAGGTATCTCAGTTCGGTGTAGGTCGT |
| | | | TCGCTCCAAGCTGGGCTGTGTGCACGAACCCCCCG |
| | | | TTCAGCCCGACCGCTGCGCCTTATCCGGTAACTATC |
| | | | GTCTTGAGTCCAACCCGGTAAGACACGACTTATCG |
| | | | CCACTGGCAGCAGCCACTGGTAACAGGATTAGCAG |
| | | | AGCGAGGTATGTAGGCGGTGCTACAGAGTTCTTGA |
| | | | AGTGGTGGCCTAACTACGGCTACACTAGAAGAACA |
| | | | GTATTTGGTATCTGCGCTCTGCTGAAGCCAGTTACC |
| | | | TTCGGAAAAAGAGTTGGTAGCTCTTGATCCGGCAA |
| | | | ACAAACCACCGCTGGTAGCGGTGGTTTTTTTGTTTG |
| | | | CAAGCAGCAGATTACGCGCAGAAAAAAAGGATCT |
| | | | CAAGAAGATCCTTTGATCTTTTCTACGGGGTCTGAC |
| | | | GCTCAGTGGAACGAAAACTCACGTTAAGGGATTTT |
| | | | GGTCATGAGATTATCAAAAAGGATCTTCACCTAGA |
| | | | TCCTTTTAAATTAAAAATGAAGTTTTAAATCAATCT |
| | | | AAAGTATATATGAGTAAACTTGGTCTGACAGTTAG |
| | | | AAAAACTCATCGAGCATCAAATGAAACTGCAATTT |
| | | | ATTCATATCAGGATTATCAATACCATATTTTTGAAA |
| | | | AAGCCGTTTCTGTAATGAAGGAGAAAACTCACCGA |
| | | | GGCAGTTCCATAGGATGGCAAGATCCTGGTATCGG |
| | | | TCTGCGATTCCGACTCGTCCAACATCAATACAACCT |
| | | | ATTAATTTCCCCTCGTCAAAAATAAGGTTATCAAGT |
| | | | GAGAAATCACCATGAGTGACGACTGAATCCGGTGA |
| | | | GAATGGCAAAAGTTTATGCATTTCTTTCCAGACTTG |
| | | | TTCAACAGGCCAGCCATTACGCTCGTCATCAAAAT |
| | | | CACTCGCATCAACCAAACCGTTATTCATTCGTGATT |
| | | | GCGCCTGAGCGAGACGAAATACGCGATCGCTGTTA |
| | | | AAAGGACAATTACAAACAGGAATCGAATGCAACC |
| | | | GGCGCAGGAACACTGCCAGCGCATCAACAATATTT |
| | | | TCACCTGAATCAGGATATTCTTCTAATACCTGGAAT |
| | | | GCTGTTTTCCCAGGGATCGCAGTGGTGAGTAACCA |
| | | | TGCATCATCAGGAGTACGGATAAAATGCTTGATGG |
| | | | TCGGAAGAGGCATAAATTCCGTCAGCCAGTTTAGT |
| | | | CTGACCATCTCATCTGTAACATCATTGGCAACGCTA |
| | | | CCTTTGCCATGTTTCAGAAACAACTCTGGCGCATCG |
| | | | GGCTTCCCATACAATCGATAGATTGTCGCACCTGAT |
| | | | TGCCCGACATTATCGCGAGCCCATTTATACCCATAT |
| | | | AAATCAGCATCCATGTTGGAATTTAATCGCGGCCT |
| | | | AGAGCAAGACGTTTCCCGTTGAATATGGCTCATAC |
| | | | TCTTCCTTTTTCAATATTATTGAAGCATTTATCAGG |
| | | | GTTATTGTCTCATGAGCGGATACATATTTGAATGTA |
| | | | TTTAGAAAAATAAACAAATAGGGGTTCCGCGCACA |
| | | | TTTCCCCGAAAAGTGCCACCTGACGTCTAAGAAAC |
| | | | CATTATTATCATGACATTAACCTATAAAAATAGGC |
| | | | GTATCACGAGGCCCTTTCGTCTCGCGCGTTTCGGTG |
| | | | ATGACGGTGAAAACCTCTGACACATGCAGCTCCCG |
| | | | GAGACGGTCACAGCTTGTCTGTAAGCGGATGCCGG |
| | | | GAGCAGACAAGCCCGTCAGGGCGCGTCAGCGGGT |
| | | | GTTGGCGGGTGTCGGGGCTGGCTTAACTATGCGGC |
| | | | ATCAGAGCAGATTGTACTGAGAGTGCACCATATGC |
| | | | GGTGTGAAATACCGCACAGATGCGTAAGGAGAAA |
| | | | ATACCGCATCAGGCGCCATTCGCCATTCAGGCTGC |
| | | | GCAACTGTTGGGAAGGGCGATCGGTGCGGGCCTCT |
| | | | TCGCTATTACGCCAGCTGGCGAAAGGGGGATGTGC |
| | | | TGCAAGGCGATTAAGTTGGGTAACGCCAGGGTTTT |
| | | | CCCAGTCACGACGTTGTAAAACGACGGCCAGTGAA |
| | | | TTGACGCGTATTGGGATCGCGCAGCCGCC |
| 35 | pYZZ415 | Rep2.Cap9- | atgccggggttttacgagattgtgattaaggtccccagcgaccttgacgagcatct |
| | | p5.L2.p5.L2.p5 | gcccggcatttctgacagctttgtgaactgggtggccgagaaggaatgggagttg |
| | | | ccgccagattctgacatggatctgaatctgattgagcaggcacccctgaccgtggc |
| | | | cgagaagctgcagcgcgactttctgacggaatggcgccgtgtgagtaaggcccc |
| | | | ggaggctcttttctttgtgcaatttgagaagggagagagctacttccacatgcacgt |
| | | | gctcgtggaaaccaccggggtgaaatccatggttttgggacgtttcctgagtcaga |
| | | | ttcgcgaaaaactgattcagagaatttaccgcgggatcgagccgactttgccaaa |
| | | | ctggttcgcggtcacaaagaccagaaatggcgccggaggcgggaacaaggtg |
| | | | gtggatgagtgctacatccccaattacttgctccccaaaacccagcctgagctcca |
| | | | gtgggcgtggactaatatggaacagtatttaagcgcctgtttgaatctcacggagc |
| | | | gtaaacggttggtggcgcagcatctgacgcacgtgtcgcagacgcaggagcag |
| | | | aacaaagagaatcagaatcccaattctgatgcgccggtgatcagatcaaaaact |
| | | | tcagccaggtacatggagctggtcgggtggctcgtggacaaggggattacctcg |
| | | | gagaagcagtggatccaggaggaccaggcctcatacatctccttcaatgcggcct |
| | | | ccaactcgcggtcccaaatcaaggctgccttggacaatgcgggaaagattatga |
| | | | gcctgactaaaaccgcccccgactacctggtgggccagcagcccgtggaggac |
| | | | atttccagcaatcggatttataaaattttggaactaaacgggtacgatccccaatat |
| | | | gcggcttccgtctttctgggatgggccacgaaaaagttcggcaagaggaacacc |
| | | | atctggctgtttgggcctgcaactaccgggaagaccaacatcgcggaggccata |
| | | | gcccacactgtgcccttctacgggtgcgtaaactggaccaatgagaactttcccttc |
| | | | aacgactgtgtcgacaagatggtgatctggtgggaggaggggaagatgaccgc |
| | | | caaggtcgtggagtcggccaaagccattctcggaggaagcaaggtgcgcgtgg |
| | | | accagaaatgcaagtcctcggcccagatagacccgactcccgtgatcgtcacctc |
| | | | caacaccaacatgtgcgccgtgattgacgggaactcaacgaccttcgaacacca |
| | | | gcagccgttgcaagaccggatgttcaaatttgaactcacccgccgtctggatcatg |
| | | | actttgggaaggtcaccaagcaggaagtcaaagactttttccggtgggcaaagg |
| | | | atcacgtggttgaggtggagcatgaattctacgtcaaaaagggtggagccaaga |
| | | | aaagacccgcccccagtgacgcagatataagtgagcccaaacgggtgcgcga |
| | | | gtcagttgcgcagccatcgacgtcagacgcggaagcttcgatcaactacgcaga |
| | | | caggtaccaaaacaaatgttctcgtcacgtgggcatgaatctgatgctgtttccctg |
| | | | cagacaatgcgagagaatgaatcagaattcaaatatctgcttcactcacggaca |
| | | | gaaagactgtttagagtgctttcccgtgtcagaatctcaacccgtttctgtcgtcaaa |
| | | | aaggcgtatcagaaactgtgctacattcatcatatcatgggaaaggtgccagacg |
| | | | cttgcactgcctgcgatctggtcaatgtggatttggatgactgcatctttgaacaata |
| | | | aatgatttaaatcaggtatggctgccgatggttatcttccagattggctcgaggacaac |
| | | | cttagtgaaggaattcgcgagtggtgggctttgaaacctggagcccctcaacccaag |
| | | | gcaaatcaacaacatcaagacaacgctcgaggtcttgtgcttccgggttacaaatacc |
| | | | ttggacccggcaacggactcgacaagggggagccggtcaacgcagcagacgcg |
| | | | gcggccctcgagcacgacaaggcctacgaccagcagctcaaggccggagacaac |
| | | | ccgtacctcaagtacaaccacgccgacgccgagttccaggagcggctcaaagaag |
| | | | atacgtcttttgggggcaacctcgggcgagcagtcttccaggccaaaaagaggcttc |
| | | | ttgaacctcttggtctggttgaggaagcggctaagacggctcctggaaagaagagg |
| | | | cctgtagagcagtctcctcaggaaccggactcctccgcgggtattggcaaatcgggt |
| | | | gcacagcccgctaaaaagagactcaatttcggtcagactggcgacacagagtcagt |
| | | | cccagaccctcaaccaatcggagaacctcccgcagccccctcaggtgtcggatctc |
| | | | ttacaatggcttcaggtggtcgcgcaccagtcgcagacaataacgaaggtgccgat |
| | | | ggagtgggtagttcctcgggaaattggcattgcgattcccaatggctgggggacaga |
| | | | gtcatcaccaccagcacccgaacctgggccctgcccacctacaacaatcacctctac |
| | | | aagcaaatctccaacagcacatctggaggatcttcaaatgacaacgcctacttcggct |
| | | | acagcaccccctgggggtattttgacttcaacagattccactgccacttctcaccacgt |
| | | | gactggcagcgactcatcaacaacaactggggattccggcctaagcgactcaacttc |
| | | | aagctcttcaacattcaggtcaaagaggttacggacaacaatggagtcaagaccatc |
| | | | gccaataaccttaccagcacggtccaggtcttcacggactcagactatcagctcccgt |
| | | | acgtgctcgggtcggctcacgagggctgcctcccgccgttcccagcggacgttttca |
| | | | tgattcctcagtacgggtatctgacgcttaatgatggaagccaggccgtgggtcgttc |
| | | | gtccttttactgcctggaatatttcccgtcgcaaatgctaagaacgggtaacaacttcc |
| | | | agttcagctacgagtttgagaacgtacctttccatagcagctacgctcacagccaaag |
| | | | cctggaccgactaatgaatccactcatcgaccaatacttgtactatctctcaaagactat |
| | | | taacggttctggacagaatcaacaaacgctaaaattcagtgtggccggacccagcaa |
| | | | catggctgtccagggaagaaactacatacctggacccagctaccgacaacaacgtg |
| | | | tctcaaccactgtgactcaaaacaacaacagcgaatttgcttggcctggagcttcttctt |
| | | | gggctctcaatggacgtaatagcttgatgaatcctggacctgctatggccagccaca |
| | | | aagaaggagaggaccgtttctttcctttgtctggatctttaatttttggcaaacaaggaa |
| | | | ctggaagagacaacgtggatgcggacaaagtcatgataaccaacgaagaagaaatt |
| | | | aaaactactaacccggtagcaacggagtcctatggacaagtggccacaaaccacca |
| | | | gagtgcccaagcacaggcgcagaccggctgggttcaaaaccaaggaatacttccg |
| | | | ggtatggtttggcaggacagagatgtgtacctgcaaggacccatttgggccaaaatt |
| | | | cctcacacggacggcaactttcacccttctccgctgatgggagggtttggaatgaag |
| | | | cacccgcctcctcagatcctcatcaaaaacacacctgtacctgcggatcctccaacg |
| | | | gccttcaacaaggacaagctgaactctttcatcacccagtattctactggccaagtca |
| | | | gcgtggagatcgagtgggagctgcagaaggaaaacagcaagcgctggaacccgg |
| | | | agatccagtacacttccaactattacaagtctaataatgttgaatttgctgttaatactga |
| | | | aggtgtatatagtgaaccccgccccattggcaccagatacctgactcgtaatctgtaa |
| | | | ttgcttgttaatcaataaaccgtttaattcgtttcagttgaACTTTGGTCTC |
| | | | TGCCCTAGGGGAGGTCCTGTATTAGAGGTCACGT |
| | | | GAGTGTTTTGCGACATTTTGCGACACCATGTGG |
| | | | TCACGCTGGGTATTTAAGCCCGAGTGAGCACGC |
| | | | AGGGTCTCCATTTTGAAGCGGGAGGTTTGAAcgc |
| | | | gcagccgccaagccgtttcagttgaactttggtctctgcgtatttctttcttatctagttt |
| | | | ccatggaggtcctgtattagaggtcacgtgagtgttttgcgacattttgcgacac |
| | | | catgtggtcacgctgggtatttaagcccgagtgagcacgcagggtctccattttg |
| | | | aagcgggaggtttgaacgcgcagccgccaagccgtttcagttgaactttggtctct |
| | | | gcgtatttctttcttatctagtttccatggaggtcctgtattagaggtcacgtgagtgt |
| | | | tttgcgacattttgcgacaccatgtggtcacgctgggtatttaagcccgagtgag |
| | | | cacgcagggtctccattttgaagcgggaggtttgaaATCCCAATGGCG |
| | | | CGCCGAGCTTGGCTCGAGCATGGTCATAGCTGTTTC |
| | | | CTGTGTGAAATTGTTATCCGCTCACAATTCCACACA |
| | | | ACATACGAGCCGGAAGCATAAAGTGTAAAGCCTGG |
| | | | GGTGCCTAATGAGTGAGCTAACTCACATTAATTGC |
| | | | GTTGCGCTCACTGCCCGCTTTCCAGTCGGGAAACCT |
| | | | GTCGTGCCAGCTGCATTAATGAATCGGCCAACGCG |
| | | | CGGGGAGAGGCGGTTTGCGTATTGGGCGCTCTTCC |
| | | | GCTTCCTCGCTCACTGACTCGCTGCGCTCGGTCGTT |
| | | | CGGCTGCGGCGAGCGGTATCAGCTCACTCAAAGGC |
| | | | GGTAATACGGTTATCCACAGAATCAGGGGATAACG |
| | | | CAGGAAAGAACATGTGAGCAAAAGGCCAGCAAAA |
| | | | GGCCAGGAACCGTAAAAAGGCCGCGTTGCTGGCGT |
| | | | TTTTCCATAGGCTCCGCCCCCCTGACGAGCATCACA |
| | | | AAAATCGACGCTCAAGTCAGAGGTGGCGAAACCCG |
| | | | ACAGGACTATAAAGATACCAGGCGTTTCCCCCTGG |
| | | | AAGCTCCCTCGTGCGCTCTCCTGTTCCGACCCTGCC |
| | | | GCTTACCGGATACCTGTCCGCCTTTCTCCCTTCGGG |
| | | | AAGCGTGGCGCTTTCTCATAGCTCACGCTGTAGGT |
| | | | ATCTCAGTTCGGTGTAGGTCGTTCGCTCCAAGCTGG |
| | | | GCTGTGTGCACGAACCCCCCGTTCAGCCCGACCGC |
| | | | TGCGCCTTATCCGGTAACTATCGTCTTGAGTCCAAC |
| | | | CCGGTAAGACACGACTTATCGCCACTGGCAGCAGC |
| | | | CACTGGTAACAGGATTAGCAGAGCGAGGTATGTAG |
| | | | GCGGTGCTACAGAGTTCTTGAAGTGGTGGCCTAAC |
| | | | TACGGCTACACTAGAAGAACAGTATTTGGTATCTG |
| | | | CGCTCTGCTGAAGCCAGTTACCTTCGGAAAAAGAG |
| | | | TTGGTAGCTCTTGATCCGGCAAACAAACCACCGCT |
| | | | GGTAGCGGTGGTTTTTTTGTTTGCAAGCAGCAGATT |
| | | | ACGCGCAGAAAAAAAGGATCTCAAGAAGATCCTTT |
| | | | GATCTTTTCTACGGGGTCTGACGCTCAGTGGAACG |
| | | | AAAACTCACGTTAAGGGATTTTGGTCATGAGATTA |
| | | | TCAAAAAGGATCTTCACCTAGATCCTTTTAAATTAA |
| | | | AAATGAAGTTTTAAATCAATCTAAAGTATATATGA |
| | | | GTAAACTTGGTCTGACAGTTAGAAAAACTCATCGA |
| | | | GCATCAAATGAAACTGCAATTTATTCATATCAGGA |
| | | | TTATCAATACCATATTTTTGAAAAAGCCGTTTCTGT |
| | | | AATGAAGGAGAAAACTCACCGAGGCAGTTCCATAG |
| | | | GATGGCAAGATCCTGGTATCGGTCTGCGATTCCGA |
| | | | CTCGTCCAACATCAATACAACCTATTAATTTCCCCT |
| | | | CGTCAAAAATAAGGTTATCAAGTGAGAAATCACCA |
| | | | TGAGTGACGACTGAATCCGGTGAGAATGGCAAAAG |
| | | | TTTATGCATTTCTTTCCAGACTTGTTCAACAGGCCA |
| | | | GCCATTACGCTCGTCATCAAAATCACTCGCATCAA |
| | | | CCAAACCGTTATTCATTCGTGATTGCGCCTGAGCGA |
| | | | GACGAAATACGCGATCGCTGTTAAAAGGACAATTA |
| | | | CAAACAGGAATCGAATGCAACCGGCGCAGGAACA |
| | | | CTGCCAGCGCATCAACAATATTTTCACCTGAATCA |
| | | | GGATATTCTTCTAATACCTGGAATGCTGTTTTCCCA |
| | | | GGGATCGCAGTGGTGAGTAACCATGCATCATCAGG |
| | | | AGTACGGATAAAATGCTTGATGGTCGGAAGAGGCA |
| | | | TAAATTCCGTCAGCCAGTTTAGTCTGACCATCTCAT |
| | | | CTGTAACATCATTGGCAACGCTACCTTTGCCATGTT |
| | | | TCAGAAACAACTCTGGCGCATCGGGCTTCCCATAC |
| | | | AATCGATAGATTGTCGCACCTGATTGCCCGACATT |
| | | | ATCGCGAGCCCATTTATACCCATATAAATCAGCAT |
| | | | CCATGTTGGAATTTAATCGCGGCCTAGAGCAAGAC |
| | | | GTTTCCCGTTGAATATGGCTCATACTCTTCCTTTTTC |
| | | | AATATTATTGAAGCATTTATCAGGGTTATTGTCTCA |
| | | | TGAGCGGATACATATTTGAATGTATTTAGAAAAAT |
| | | | AAACAAATAGGGGTTCCGCGCACATTTCCCCGAAA |
| | | | AGTGCCACCTGACGTCTAAGAAACCATTATTATCA |
| | | | TGACATTAACCTATAAAAATAGGCGTATCACGAGG |
| | | | CCCTTTCGTCTCGCGCGTTTCGGTGATGACGGTGAA |
| | | | AACCTCTGACACATGCAGCTCCCGGAGACGGTCAC |
| | | | AGCTTGTCTGTAAGCGGATGCCGGGAGCAGACAAG |
| | | | CCCGTCAGGGCGCGTCAGCGGGTGTTGGCGGGTGT |
| | | | CGGGGCTGGCTTAACTATGCGGCATCAGAGCAGAT |
| | | | TGTACTGAGAGTGCACCATATGCGGTGTGAAATAC |
| | | | CGCACAGATGCGTAAGGAGAAAATACCGCATCAG |
| | | | GCGCCATTCGCCATTCAGGCTGCGCAACTGTTGGG |
| | | | AAGGGCGATCGGTGCGGGCCTCTTCGCTATTACGC |
| | | | CAGCTGGCGAAAGGGGGATGTGCTGCAAGGCGATT |
| | | | AAGTTGGGTAACGCCAGGGTTTTCCCAGTCACGAC |
| | | | GTTGTAAAACGACGGCCAGTGAATTGACGCGTATT |
| | | | GGGATCGCGCAGCCGCC |
| 36 | pYZZ278 | p5v1-Rep2Ca9- | TCGCGCGTTTCGGTGATGACGGTGAAAACCTCTGA |
| | | P5 | CACATGCAGCTCCCGGAGACGGTCACAGCTTGTCT |
| | | | GTAAGCGGATGCCGGGAGCAGACAAGCCCGTCAG |
| | | | GGCGCGTCAGCGGGTGTTGGCGGGTGTCGGGGCTG |
| | | | GCTTAACTATGCGGCATCAGAGCAGATTGTACTGA |
| | | | GAGTGCACCATATGCGGTGTGAAATACCGCACAGA |
| | | | TGCGTAAGGAGAAAATACCGCATCAGGCGCCATTC |
| | | | GCCATTCAGGCTGCGCAACTGTTGGGAAGGGCGAT |
| | | | CGGTGCGGGCCTCTTCGCTATTACGCCAGCTGGCG |
| | | | AAAGGGGGATGTGCTGCAAGGCGATTAAGTTGGGT |
| | | | AACGCCAGGGTTTTCCCAGTCACGACGTTGTAAAA |
| | | | CGACGGCCAGTGAATTGACGCGTATTGggatAGGGT |
| | | | CTCCATTTTGAAGCGGGAGGTTTGAACGCGCAG |
| | | | CCGCCATGccggggttttacgagattgtgattaaggtccccagcgaccttga |
| | | | cgagcatctgcccggcatttctgacagctttgtgaactgggggccgagaaggaa |
| | | | tgggagttgccgccagattctgacatggatctgaatctgattgagcaggcacccct |
| | | | gaccgtggccgagaagctgcagcgcgactttctgacggaatggcgccgtgtgag |
| | | | taaggccccggaggctcttttctttgtgcaatttgagaagggagagagctacttcca |
| | | | catgcacgtgctcgtggaaaccaccggggTGAAATCCATGGTTTTG |
| | | | GGACGTTTCCTGAGTCAGATTCGCGAAAAACTGATTCA |
| | | | GAGAATTTACCGCGGGATCGAGCCGACTTTGCCAAAC |
| | | | TGGTTCGCGGTCACAAAGACCAGAAATGGCGCCGGAG |
| | | | GCGGGAACAAGGTGGTGGATGAGTGCTACATCCCCAA |
| | | | TTACTTGCTCCCCAAAACCCAGCCTGAGCTCCAGTGG |
| | | | GCGTGGACTAATATGGAACAGTATTTAAGCGCCTGTTT |
| | | | GAATCTCACGGAGCGTAAACGGTTGGTGGCGCAGCAT |
| | | | CTGACGCACGTGTCGCAGACGCAGGAGCAGAACAAAG |
| | | | AGAATCAGAATCCCAATTCTGATGCGCCGGTGATCAGA |
| | | | TCAAAAACTTCAGCCAGGTACATGGAGCTGGTCGGGT |
| | | | GGCTCGTGGACAAGGGGATTACCTCGGAGAAGCAGTG |
| | | | GATCCAGGAGGACCAGGCCTCATACATCTCCTTCAATG |
| | | | CGGCCTCCAACTCGCGGTCCCAAATCAAGGCTGCCTT |
| | | | GGACAATGCGGGAAAGATTATGAGCCTGACTAAAACC |
| | | | GCCCCCGACTACCTGGTGGGCCAGCAGCCCGTGGAG |
| | | | GACATTTCCAGCAATCGGATTTATAAAATTTTGGAACTA |
| | | | AACGGGTACGATCCCCAATATGCGGCTTCCGTCTTTCT |
| | | | GGGATGGGCCACGAAAAAGTTCGGCAAGAGGAACACC |
| | | | ATCTGGCTGTTTGGGCCTGCAACTACCGGGAAGACCA |
| | | | ACATCGCGGAGGCCATAGCCCACACTGTGCCCTTCTA |
| | | | CGGGTGCGTAAACTGGACCAATGAGAACTTTCCCTTCA |
| | | | ACGACTGTGTCGACAAGATGGTGATCTGGTGGGAGGA |
| | | | GGGGAAGATGACCGCCAAGGTCGTGGAGTCGGCCAA |
| | | | AGCCATTCTCGGAGGAAGCAAGGTGCGCGTGGACCA |
| | | | GAAATGCAAGTCCTCGGCCCAGATAGACCCGACTCCC |
| | | | GTGATCGTCACCTCCAACACCAACATGTGCGCCGTGA |
| | | | TTGACGGGAACTCAACGACCTTCGAACACCAGCAGCC |
| | | | GTTGCAAGACCGGATGTTCAAATTTGAACTCACCCGCC |
| | | | GTCTGGATCATGACTTTGGGAAGGTCACCAAGCAGGA |
| | | | AGTCAAAGACTTTTTCCGGTGGGCAAAGGATCACGTG |
| | | | GTTGAGGTGGAGCATGAATTCTACGTCAAAAAGGGTG |
| | | | GAGCCAAGAAAAGACCCGCCCCCAGTGACGCAGATAT |
| | | | AAGTGAGCCCAAACGGGTGCGCGAGTCAGTTGCGCA |
| | | | GCCATCGACGTCAGACGCGGAAGCTTCGATCAACTAC |
| | | | GCAGACAGGTACCAAAACAAATGTTCTCGTCACGTGG |
| | | | GCATGAATCTGATGCTGTTTCCCTGCAGACAATGCGAG |
| | | | AGAATGAATCAGAATTCAAATATCTGCTTCACTCACGG |
| | | | ACAGAAAGACTGTTTAGAGTGCTTTCCCGTGTCAGAAT |
| | | | CTCAACCCGTTTCTGTCGTCAAAAAGGCGTATCAGAAA |
| | | | CTGTGCTACATTCATCATATCATGGGAAAGGTGCCAGA |
| | | | CGCTTGCACTGCCTGCGATCTGGTCAATGTGGATTTG |
| | | | GATGACTGCATCTTTGAACAATAAATGATTTAAATCA |
| | | | GGTATGGCTGCCGATGGTTATCTTCCAGATTGGCTC |
| | | | GAGGACAACCTTAGTGAAGGAATTCGCGAGTGGTG |
| | | | GGCTTTGAAACCTGGAGCCCCTCAACCCAAGGCAA |
| | | | ATCAACAACATCAAGACAACGCTCGAGGTCTTGTG |
| | | | CTTCCGGGTTACAAATACCTTGGACCCGGCAACGG |
| | | | ACTCGACAAGGGGGAGCCGGTCAACGCAGCAGAC |
| | | | GCGGCGGCCCTCGAGCACGACAAGGCCTACGACCA |
| | | | GCAGCTCAAGGCCGGAGACAACCCGTACCTCAAGT |
| | | | ACAACCACGCCGACGCCGAGTTCCAGGAGCGGCTC |
| | | | AAAGAAGATACGTCTTTTGGGGGCAACCTCGGGCG |
| | | | AGCAGTCTTCCAGGCCAAAAAGAGGCTTCTTGAAC |
| | | | CTCTTGGTCTGGTTGAGGAAGCGGCTAAGACGGCT |
| | | | CCTGGAAAGAAGAGGCCTGTAGAGCAGTCTCCTCA |
| | | | GGAACCGGACTCCTCCGCGGGTATTGGCAAATCGG |
| | | | GTGCACAGCCCGCTAAAAAGAGACTCAATTTCGGT |
| | | | CAGACTGGCGACACAGAGTCAGTCCCAGACCCTCA |
| | | | ACCAATCGGAGAACCTCCCGCAGCCCCCTCAGGTG |
| | | | TGGGATCTCTTACAATGGCTTCAGGTGGTGGCGCA |
| | | | CCAGTGGCAGACAATAACGAAGGTGCCGATGGAGT |
| | | | GGGTAGTTCCTCGGGAAATTGGCATTGCGATTCCC |
| | | | AATGGCTGGGGGACAGAGTCATCACCACCAGCACC |
| | | | CGAACCTGGGCCCTGCCCACCTACAACAATCACCT |
| | | | CTACAAGCAAATCTCCAACAGCACATCTGGAGGAT |
| | | | CTTCAAATGACAACGCCTACTTCGGCTACAGCACC |
| | | | CCCTGGGGGTATTTTGACTTCAACAGATTCCACTGC |
| | | | CACTTCTCACCACGTGACTGGCAGCGACTCATCAA |
| | | | CAACAACTGGGGATTCCGGCCTAAGCGACTCAACT |
| | | | TCAAGCTCTTCAACATTCAGGTCAAAGAGGTTACG |
| | | | GACAACAATGGAGTCAAGACCATCGCCAATAACCT |
| | | | TACCAGCACGGTCCAGGTCTTCACGGACTCAGACT |
| | | | ATCAGCTCCCGTACGTGCTCGGGTCGGCTCACGAG |
| | | | GGCTGCCTCCCGCCGTTCCCAGCGGACGTTTTCATG |
| | | | ATTCCTCAGTACGGGTATCTGACGCTTAATGATGG |
| | | | AAGCCAGGCCGTGGGTCGTTCGTCCTTTTACTGCCT |
| | | | GGAATATTTCCCGTCGCAAATGCTAAGAACGGGTA |
| | | | ACAACTTCCAGTTCAGCTACGAGTTTGAGAACGTA |
| | | | CCTTTCCATAGCAGCTACGCTCACAGCCAAAGCCT |
| | | | GGACCGACTAATGAATCCACTCATCGACCAATACT |
| | | | TGTACTATCTCTCAAAGACTATTAACGGTTCTGGAC |
| | | | AGAATCAACAAACGCTAAAATTCAGTGTGGCCGGA |
| | | | CCCAGCAACATGGCTGTCCAGGGAAGAAACTACAT |
| | | | ACCTGGACCCAGCTACCGACAACAACGTGTCTCAA |
| | | | CCACTGTGACTCAAAACAACAACAGCGAATTTGCT |
| | | | TGGCCTGGAGCTTCTTCTTGGGCTCTCAATGGACGT |
| | | | AATAGCTTGATGAATCCTGGACCTGCTATGGCCAG |
| | | | CCACAAAGAAGGAGAGGACCGTTTCTTTCCTTTGT |
| | | | CTGGATCTTTAATTTTTGGCAAACAAGGAACTGGA |
| | | | AGAGACAACGTGGATGCGGACAAAGTCATGATAA |
| | | | CCAACGAAGAAGAAATTAAAACTACTAACCCGGTA |
| | | | GCAACGGAGTCCTATGGACAAGTGGCCACAAACCA |
| | | | CCAGAGTGCCCAAGCACAGGCGCAGACCGGCTGG |
| | | | GTTCAAAACCAAGGAATACTTCCGGGTATGGTTTG |
| | | | GCAGGACAGAGATGTGTACCTGCAAGGACCCATTT |
| | | | GGGCCAAAATTCCTCACACGGACGGCAACTTTCAC |
| | | | CCTTCTCCGCTGATGGGAGGGTTTGGAATGAAGCA |
| | | | CCCGCCTCCTCAGATCCTCATCAAAAACACACCTGT |
| | | | ACCTGCGGATCCTCCAACGGCCTTCAACAAGGACA |
| | | | AGCTGAACTCTTTCATCACCCAGTATTCTACTGGCC |
| | | | AAGTCAGCGTGGAGATCGAGTGGGAGCTGCAGAA |
| | | | GGAAAACAGCAAGCGCTGGAACCCGGAGATCCAG |
| | | | TACACTTCCAACTATTACAAGTCTAATAATGTTGAA |
| | | | TTTGCTGTTAATACTGAAGGTGTATATAGTGAACCC |
| | | | CGCCCCATTGGCACCAGATACCTGACTCGTAATCT |
| | | | GTAATTGCTTGTTAATCAATAAACCGTTTAATTC |
| | | | GTTTCAGTTGAACTTTGGTCTCTGCGGAGGTCCT |
| | | | GTATTAGAGGTCACGTGAGTGTTTTGCGACATT |
| | | | TTGCGACACCATGTGGTCACGCTGGGTATTTAA |
| | | | GCCCGAGTGAGCACGCAGGGTCTCCATTTTGAA |
| | | | GCGGGAGGTTTGAAATCCCAATGGCGCGCCGAGC |
| | | | TTGGCTCGAGCATGGTCATAGCTGTTTCCTGTGTGA |
| | | | AATTGTTATCCGCTCACAATTCCACACAACATACG |
| | | | AGCCGGAAGCATAAAGTGTAAAGCCTGGGGTGCCT |
| | | | AATGAGTGAGCTAACTCACATTAATTGCGTTGCGC |
| | | | TCACTGCCCGCTTTCCAGTCGGGAAACCTGTCGTGC |
| | | | CAGCTGCATTAATGAATCGGCCAACGCGCGGGGAG |
| | | | AGGCGGTTTGCGTATTGGGCGCTCTTCCGCTTCCTC |
| | | | GCTCACTGACTCGCTGCGCTCGGTCGTTCGGCTGCG |
| | | | GCGAGCGGTATCAGCTCACTCAAAGGCGGTAATAC |
| | | | GGTTATCCACAGAATCAGGGGATAACGCAGGAAA |
| | | | GAACATGTGAGCAAAAGGCCAGCAAAAGGCCAGG |
| | | | AACCGTAAAAAGGCCGCGTTGCTGGCGTTTTTCCA |
| | | | TAGGCTCCGCCCCCCTGACGAGCATCACAAAAATC |
| | | | GACGCTCAAGTCAGAGGTGGCGAAACCCGACAGG |
| | | | ACTATAAAGATACCAGGCGTTTCCCCCTGGAAGCT |
| | | | CCCTCGTGCGCTCTCCTGTTCCGACCCTGCCGCTTA |
| | | | CCGGATACCTGTCCGCCTTTCTCCCTTCGGGAAGCG |
| | | | TGGCGCTTTCTCATAGCTCACGCTGTAGGTATCTCA |
| | | | GTTCGGTGTAGGTCGTTCGCTCCAAGCTGGGCTGTG |
| | | | TGCACGAACCCCCCGTTCAGCCCGACCGCTGCGCC |
| | | | TTATCCGGTAACTATCGTCTTGAGTCCAACCCGGTA |
| | | | AGACACGACTTATCGCCACTGGCAGCAGCCACTGG |
| | | | TAACAGGATTAGCAGAGCGAGGTATGTAGGCGGTG |
| | | | CTACAGAGTTCTTGAAGTGGTGGCCTAACTACGGC |
| | | | TACACTAGAAGAACAGTATTTGGTATCTGCGCTCT |
| | | | GCTGAAGCCAGTTACCTTCGGAAAAAGAGTTGGTA |
| | | | GCTCTTGATCCGGCAAACAAACCACCGCTGGTAGC |
| | | | GGTGGTTTTTTTGTTTGCAAGCAGCAGATTACGCGC |
| | | | AGAAAAAAAGGATCTCAAGAAGATCCTTTGATCTT |
| | | | TTCTACGGGGTCTGACGCTCAGTGGAACGAAAACT |
| | | | CACGTTAAGGGATTTTGGTCATGAGATTATCAAAA |
| | | | AGGATCTTCACCTAGATCCTTTTAAATTAAAAATGA |
| | | | AGTTTTAAATCAATCTAAAGTATATATGAGTAAAC |
| | | | TTGGTCTGACAGTTAGAAAAACTCATCGAGCATCA |
| | | | AATGAAACTGCAATTTATTCATATCAGGATTATCA |
| | | | ATACCATATTTTTGAAAAAGCCGTTTCTGTAATGAA |
| | | | GGAGAAAACTCACCGAGGCAGTTCCATAGGATGGC |
| | | | AAGATCCTGGTATCGGTCTGCGATTCCGACTCGTCC |
| | | | AACATCAATACAACCTATTAATTTCCCCTCGTCAAA |
| | | | AATAAGGTTATCAAGTGAGAAATCACCATGAGTGA |
| | | | CGACTGAATCCGGTGAGAATGGCAAAAGTTTATGC |
| | | | ATTTCTTTCCAGACTTGTTCAACAGGCCAGCCATTA |
| | | | CGCTCGTCATCAAAATCACTCGCATCAACCAAACC |
| | | | GTTATTCATTCGTGATTGCGCCTGAGCGAGACGAA |
| | | | ATACGCGATCGCTGTTAAAAGGACAATTACAAACA |
| | | | GGAATCGAATGCAACCGGCGCAGGAACACTGCCA |
| | | | GCGCATCAACAATATTTTCACCTGAATCAGGATATT |
| | | | CTTCTAATACCTGGAATGCTGTTTTCCCAGGGATCG |
| | | | CAGTGGTGAGTAACCATGCATCATCAGGAGTACGG |
| | | | ATAAAATGCTTGATGGTCGGAAGAGGCATAAATTC |
| | | | CGTCAGCCAGTTTAGTCTGACCATCTCATCTGTAAC |
| | | | ATCATTGGCAACGCTACCTTTGCCATGTTTCAGAAA |
| | | | CAACTCTGGCGCATCGGGCTTCCCATACAATCGAT |
| | | | AGATTGTCGCACCTGATTGCCCGACATTATCGCGA |
| | | | GCCCATTTATACCCATATAAATCAGCATCCATGTTG |
| | | | GAATTTAATCGCGGCCTAGAGCAAGACGTTTCCCG |
| | | | TTGAATATGGCTCATACTCTTCCTTTTTCAATATTAT |
| | | | TGAAGCATTTATCAGGGTTATTGTCTCATGAGCGG |
| | | | ATACATATTTGAATGTATTTAGAAAAATAAACAAA |
| | | | TAGGGGTTCCGCGCACATTTCCCCGAAAAGTGCCA |
| | | | CCTGACGTCTAAGAAACCATTATTATCATGACATTA |
| | | | ACCTATAAAAATAGGCGTATCACGAGGCCCTTTCG |
| | | | TC |
| 37 | pYZZ279 | p5v2-Rep2Ca9- | TCGCGCGTTTCGGTGATGACGGTGAAAACCTCTGA |
| | | P5 | CACATGCAGCTCCCGGAGACGGTCACAGCTTGTCT |
| | | | GTAAGCGGATGCCGGGAGCAGACAAGCCCGTCAG |
| | | | GGCGCGTCAGCGGGTGTTGGCGGGTGTCGGGGCTG |
| | | | GCTTAACTATGCGGCATCAGAGCAGATTGTACTGA |
| | | | GAGTGCACCATATGCGGTGTGAAATACCGCACAGA |
| | | | TGCGTAAGGAGAAAATACCGCATCAGGCGCCATTC |
| | | | GCCATTCAGGCTGCGCAACTGTTGGGAAGGGCGAT |
| | | | CGGTGCGGGCCTCTTCGCTATTACGCCAGCTGGCG |
| | | | AAAGGGGGATGTGCTGCAAGGCGATTAAGTTGGGT |
| | | | AACGCCAGGGTTTTCCCAGTCACGACGTTGTAAAA |
| | | | CGACGGCCAGTGAATTGACGCGTATTGggatGCCCG |
| | | | AGTGAGCACGCAGGTTTGAACGCGCAGCCGCCA |
| | | | TGccggggttttacgagattgtgattaaggtccccagcgaccttgacgagcatct |
| | | | gcccggcatttctgacagctttgtgaactgggtggccgagaaggaatgggagttg |
| | | | ccgccagattctgacatggatctgaatctgattgagcaggcacccctgaccgtggc |
| | | | cgagaagctgcagcgcgactttctgacggaatggcgccgtgtgagtaaggcccc |
| | | | ggaggctcttttctttgtgcaatttgagaagggagagagctacttccacatgcacgt |
| | | | gctcgtggaaaccaccggggTGAAATCCATGGTTTTGGGACGTT |
| | | | TCCTGAGTCAGATTCGCGAAAAACTGATTCAGAGAATT |
| | | | TACCGCGGGATCGAGCCGACTTTGCCAAACTGGTTCG |
| | | | CGGTCACAAAGACCAGAAATGGCGCCGGAGGCGGGA |
| | | | ACAAGGTGGTGGATGAGTGCTACATCCCCAATTACTTG |
| | | | CTCCCCAAAACCCAGCCTGAGCTCCAGTGGGCGTGGA |
| | | | CTAATATGGAACAGTATTTAAGCGCCTGTTTGAATCTCA |
| | | | CGGAGCGTAAACGGTTGGTGGCGCAGCATCTGACGCA |
| | | | CGTGTCGCAGACGCAGGAGCAGAACAAAGAGAATCAG |
| | | | AATCCCAATTCTGATGCGCCGGTGATCAGATCAAAAAC |
| | | | TTCAGCCAGGTACATGGAGCTGGTCGGGTGGCTCGTG |
| | | | GACAAGGGGATTACCTCGGAGAAGCAGTGGATCCAGG |
| | | | AGGACCAGGCCTCATACATCTCCTTCAATGCGGCCTC |
| | | | CAACTCGCGGTCCCAAATCAAGGCTGCCTTGGACAAT |
| | | | GCGGGAAAGATTATGAGCCTGACTAAAACCGCCCCCG |
| | | | ACTACCTGGTGGGCCAGCAGCCCGTGGAGGACATTTC |
| | | | CAGCAATCGGATTTATAAAATTTTGGAACTAAACGGGT |
| | | | ACGATCCCCAATATGCGGCTTCCGTCTTTCTGGGATGG |
| | | | GCCACGAAAAAGTTCGGCAAGAGGAACACCATCTGGC |
| | | | TGTTTGGGCCTGCAACTACCGGGAAGACCAACATCGC |
| | | | GGAGGCCATAGCCCACACTGTGCCCTTCTACGGGTGC |
| | | | GTAAACTGGACCAATGAGAACTTTCCCTTCAACGACTG |
| | | | TGTCGACAAGATGGTGATCTGGTGGGAGGAGGGGAA |
| | | | GATGACCGCCAAGGTCGTGGAGTCGGCCAAAGCCATT |
| | | | CTCGGAGGAAGCAAGGTGCGCGTGGACCAGAAATGC |
| | | | AAGTCCTCGGCCCAGATAGACCCGACTCCCGTGATCG |
| | | | TCACCTCCAACACCAACATGTGCGCCGTGATTGACGG |
| | | | GAACTCAACGACCTTCGAACACCAGCAGCCGTTGCAA |
| | | | GACCGGATGTTCAAATTTGAACTCACCCGCCGTCTGGA |
| | | | TCATGACTTTGGGAAGGTCACCAAGCAGGAAGTCAAA |
| | | | GACTTTTTCCGGTGGGCAAAGGATCACGTGGTTGAGG |
| | | | TGGAGCATGAATTCTACGTCAAAAAGGGTGGAGCCAA |
| | | | GAAAAGACCCGCCCCCAGTGACGCAGATATAAGTGAG |
| | | | CCCAAACGGGTGCGCGAGTCAGTTGCGCAGCCATCGA |
| | | | CGTCAGACGCGGAAGCTTCGATCAACTACGCAGACAG |
| | | | GTACCAAAACAAATGTTCTCGTCACGTGGGCATGAATC |
| | | | TGATGCTGTTTCCCTGCAGACAATGCGAGAGAATGAAT |
| | | | CAGAATTCAAATATCTGCTTCACTCACGGACAGAAAGA |
| | | | CTGTTTAGAGTGCTTTCCCGTGTCAGAATCTCAACCCG |
| | | | TTTCTGTCGTCAAAAAGGCGTATCAGAAACTGTGCTAC |
| | | | ATTCATCATATCATGGGAAAGGTGCCAGACGCTTGCAC |
| | | | TGCCTGCGATCTGGTCAATGTGGATTTGGATGACTGCA |
| | | | TCTTTGAACAATAAATGATTTAAATCAGGTATGGCTG |
| | | | CCGATGGTTATCTTCCAGATTGGCTCGAGGACAAC |
| | | | CTTAGTGAAGGAATTCGCGAGTGGTGGGCTTTGAA |
| | | | ACCTGGAGCCCCTCAACCCAAGGCAAATCAACAAC |
| | | | ATCAAGACAACGCTCGAGGTCTTGTGCTTCCGGGT |
| | | | TACAAATACCTTGGACCCGGCAACGGACTCGACAA |
| | | | GGGGGAGCCGGTCAACGCAGCAGACGCGGCGGCC |
| | | | CTCGAGCACGACAAGGCCTACGACCAGCAGCTCAA |
| | | | GGCCGGAGACAACCCGTACCTCAAGTACAACCACG |
| | | | CCGACGCCGAGTTCCAGGAGCGGCTCAAAGAAGAT |
| | | | ACGTCTTTTGGGGGCAACCTCGGGCGAGCAGTCTT |
| | | | CCAGGCCAAAAAGAGGCTTCTTGAACCTCTTGGTC |
| | | | TGGTTGAGGAAGCGGCTAAGACGGCTCCTGGAAAG |
| | | | AAGAGGCCTGTAGAGCAGTCTCCTCAGGAACCGGA |
| | | | CTCCTCCGCGGGTATTGGCAAATCGGGTGCACAGC |
| | | | CCGCTAAAAAGAGACTCAATTTCGGTCAGACTGGC |
| | | | GACACAGAGTCAGTCCCAGACCCTCAACCAATCGG |
| | | | AGAACCTCCCGCAGCCCCCTCAGGTGTGGGATCTC |
| | | | TTACAATGGCTTCAGGTGGTGGCGCACCAGTGGCA |
| | | | GACAATAACGAAGGTGCCGATGGAGTGGGTAGTTC |
| | | | CTCGGGAAATTGGCATTGCGATTCCCAATGGCTGG |
| | | | GGGACAGAGTCATCACCACCAGCACCCGAACCTGG |
| | | | GCCCTGCCCACCTACAACAATCACCTCTACAAGCA |
| | | | AATCTCCAACAGCACATCTGGAGGATCTTCAAATG |
| | | | ACAACGCCTACTTCGGCTACAGCACCCCCTGGGGG |
| | | | TATTTTGACTTCAACAGATTCCACTGCCACTTCTCA |
| | | | CCACGTGACTGGCAGCGACTCATCAACAACAACTG |
| | | | GGGATTCCGGCCTAAGCGACTCAACTTCAAGCTCT |
| | | | TCAACATTCAGGTCAAAGAGGTTACGGACAACAAT |
| | | | GGAGTCAAGACCATCGCCAATAACCTTACCAGCAC |
| | | | GGTCCAGGTCTTCACGGACTCAGACTATCAGCTCC |
| | | | CGTACGTGCTCGGGTCGGCTCACGAGGGCTGCCTC |
| | | | CCGCCGTTCCCAGCGGACGTTTTCATGATTCCTCAG |
| | | | TACGGGTATCTGACGCTTAATGATGGAAGCCAGGC |
| | | | CGTGGGTCGTTCGTCCTTTTACTGCCTGGAATATTT |
| | | | CCCGTCGCAAATGCTAAGAACGGGTAACAACTTCC |
| | | | AGTTCAGCTACGAGTTTGAGAACGTACCTTTCCATA |
| | | | GCAGCTACGCTCACAGCCAAAGCCTGGACCGACTA |
| | | | ATGAATCCACTCATCGACCAATACTTGTACTATCTC |
| | | | TCAAAGACTATTAACGGTTCTGGACAGAATCAACA |
| | | | AACGCTAAAATTCAGTGTGGCCGGACCCAGCAACA |
| | | | TGGCTGTCCAGGGAAGAAACTACATACCTGGACCC |
| | | | AGCTACCGACAACAACGTGTCTCAACCACTGTGAC |
| | | | TCAAAACAACAACAGCGAATTTGCTTGGCCTGGAG |
| | | | CTTCTTCTTGGGCTCTCAATGGACGTAATAGCTTGA |
| | | | TGAATCCTGGACCTGCTATGGCCAGCCACAAAGAA |
| | | | GGAGAGGACCGTTTCTTTCCTTTGTCTGGATCTTTA |
| | | | ATTTTTGGCAAACAAGGAACTGGAAGAGACAACGT |
| | | | GGATGCGGACAAAGTCATGATAACCAACGAAGAA |
| | | | GAAATTAAAACTACTAACCCGGTAGCAACGGAGTC |
| | | | CTATGGACAAGTGGCCACAAACCACCAGAGTGCCC |
| | | | AAGCACAGGCGCAGACCGGCTGGGTTCAAAACCA |
| | | | AGGAATACTTCCGGGTATGGTTTGGCAGGACAGAG |
| | | | ATGTGTACCTGCAAGGACCCATTTGGGCCAAAATT |
| | | | CCTCACACGGACGGCAACTTTCACCCTTCTCCGCTG |
| | | | ATGGGAGGGTTTGGAATGAAGCACCCGCCTCCTCA |
| | | | GATCCTCATCAAAAACACACCTGTACCTGCGGATC |
| | | | CTCCAACGGCCTTCAACAAGGACAAGCTGAACTCT |
| | | | TTCATCACCCAGTATTCTACTGGCCAAGTCAGCGTG |
| | | | GAGATCGAGTGGGAGCTGCAGAAGGAAAACAGCA |
| | | | AGCGCTGGAACCCGGAGATCCAGTACACTTCCAAC |
| | | | TATTACAAGTCTAATAATGTTGAATTTGCTGTTAAT |
| | | | ACTGAAGGTGTATATAGTGAACCCCGCCCCATTGG |
| | | | CACCAGATACCTGACTCGTAATCTGTAATTGCTTG |
| | | | TTAATCAATAAACCGTTTAATTCGTTTCAGTTGA |
| | | | ACTTTGGTCTCTGCGGAGGTCCTGTATTAGAGGT |
| | | | CACGTGAGTGTTTTGCGACATTTTGCGACACCA |
| | | | TGTGGTCACGCTGGGTATTTAAGCCCGAGTGAG |
| | | | CACGCAGGGTCTCCATTTTGAAGCGGGAGGTTT |
| | | | GAAATCCCAATGGCGCGCCGAGCTTGGCTCGAGCA |
| | | | TGGTCATAGCTGTTTCCTGTGTGAAATTGTTATCCG |
| | | | CTCACAATTCCACACAACATACGAGCCGGAAGCAT |
| | | | AAAGTGTAAAGCCTGGGGTGCCTAATGAGTGAGCT |
| | | | AACTCACATTAATTGCGTTGCGCTCACTGCCCGCTT |
| | | | TCCAGTCGGGAAACCTGTCGTGCCAGCTGCATTAA |
| | | | TGAATCGGCCAACGCGCGGGGAGAGGCGGTTTGCG |
| | | | TATTGGGCGCTCTTCCGCTTCCTCGCTCACTGACTC |
| | | | GCTGCGCTCGGTCGTTCGGCTGCGGCGAGCGGTAT |
| | | | CAGCTCACTCAAAGGCGGTAATACGGTTATCCACA |
| | | | GAATCAGGGGATAACGCAGGAAAGAACATGTGAG |
| | | | CAAAAGGCCAGCAAAAGGCCAGGAACCGTAAAAA |
| | | | GGCCGCGTTGCTGGCGTTTTTCCATAGGCTCCGCCC |
| | | | CCCTGACGAGCATCACAAAAATCGACGCTCAAGTC |
| | | | AGAGGTGGCGAAACCCGACAGGACTATAAAGATA |
| | | | CCAGGCGTTTCCCCCTGGAAGCTCCCTCGTGCGCTC |
| | | | TCCTGTTCCGACCCTGCCGCTTACCGGATACCTGTC |
| | | | CGCCTTTCTCCCTTCGGGAAGCGTGGCGCTTTCTCA |
| | | | TAGCTCACGCTGTAGGTATCTCAGTTCGGTGTAGGT |
| | | | CGTTCGCTCCAAGCTGGGCTGTGTGCACGAACCCC |
| | | | CCGTTCAGCCCGACCGCTGCGCCTTATCCGGTAACT |
| | | | ATCGTCTTGAGTCCAACCCGGTAAGACACGACTTA |
| | | | TCGCCACTGGCAGCAGCCACTGGTAACAGGATTAG |
| | | | CAGAGCGAGGTATGTAGGCGGTGCTACAGAGTTCT |
| | | | TGAAGTGGTGGCCTAACTACGGCTACACTAGAAGA |
| | | | ACAGTATTTGGTATCTGCGCTCTGCTGAAGCCAGTT |
| | | | ACCTTCGGAAAAAGAGTTGGTAGCTCTTGATCCGG |
| | | | CAAACAAACCACCGCTGGTAGCGGTGGTTTTTTTGT |
| | | | TTGCAAGCAGCAGATTACGCGCAGAAAAAAAGGA |
| | | | TCTCAAGAAGATCCTTTGATCTTTTCTACGGGGTCT |
| | | | GACGCTCAGTGGAACGAAAACTCACGTTAAGGGAT |
| | | | TTTGGTCATGAGATTATCAAAAAGGATCTTCACCTA |
| | | | GATCCTTTTAAATTAAAAATGAAGTTTTAAATCAAT |
| | | | CTAAAGTATATATGAGTAAACTTGGTCTGACAGTT |
| | | | AGAAAAACTCATCGAGCATCAAATGAAACTGCAAT |
| | | | TTATTCATATCAGGATTATCAATACCATATTTTTGA |
| | | | AAAAGCCGTTTCTGTAATGAAGGAGAAAACTCACC |
| | | | GAGGCAGTTCCATAGGATGGCAAGATCCTGGTATC |
| | | | GGTCTGCGATTCCGACTCGTCCAACATCAATACAA |
| | | | CCTATTAATTTCCCCTCGTCAAAAATAAGGTTATCA |
| | | | AGTGAGAAATCACCATGAGTGACGACTGAATCCGG |
| | | | TGAGAATGGCAAAAGTTTATGCATTTCTTTCCAGAC |
| | | | TTGTTCAACAGGCCAGCCATTACGCTCGTCATCAA |
| | | | AATCACTCGCATCAACCAAACCGTTATTCATTCGTG |
| | | | ATTGCGCCTGAGCGAGACGAAATACGCGATCGCTG |
| | | | TTAAAAGGACAATTACAAACAGGAATCGAATGCAA |
| | | | CCGGCGCAGGAACACTGCCAGCGCATCAACAATAT |
| | | | TTTCACCTGAATCAGGATATTCTTCTAATACCTGGA |
| | | | ATGCTGTTTTCCCAGGGATCGCAGTGGTGAGTAAC |
| | | | CATGCATCATCAGGAGTACGGATAAAATGCTTGAT |
| | | | GGTCGGAAGAGGCATAAATTCCGTCAGCCAGTTTA |
| | | | GTCTGACCATCTCATCTGTAACATCATTGGCAACGC |
| | | | TACCTTTGCCATGTTTCAGAAACAACTCTGGCGCAT |
| | | | CGGGCTTCCCATACAATCGATAGATTGTCGCACCT |
| | | | GATTGCCCGACATTATCGCGAGCCCATTTATACCCA |
| | | | TATAAATCAGCATCCATGTTGGAATTTAATCGCGG |
| | | | CCTAGAGCAAGACGTTTCCCGTTGAATATGGCTCA |
| | | | TACTCTTCCTTTTTCAATATTATTGAAGCATTTATCA |
| | | | GGGTTATTGTCTCATGAGCGGATACATATTTGAATG |
| | | | TATTTAGAAAAATAAACAAATAGGGGTTCCGCGCA |
| | | | CATTTCCCCGAAAAGTGCCACCTGACGTCTAAGAA |
| | | | ACCATTATTATCATGACATTAACCTATAAAAATAG |
| | | | GCGTATCACGAGGCCCTTTCGTC |
| 38 | pYZZ280 | p5v3-Rep2Ca9- | TCGCGCGTTTCGGTGATGACGGTGAAAACCTCTGA |
| | | P5 | CACATGCAGCTCCCGGAGACGGTCACAGCTTGTCT |
| | | | GTAAGCGGATGCCGGGAGCAGACAAGCCCGTCAG |
| | | | GGCGCGTCAGCGGGTGTTGGCGGGTGTCGGGGCTG |
| | | | GCTTAACTATGCGGCATCAGAGCAGATTGTACTGA |
| | | | GAGTGCACCATATGCGGTGTGAAATACCGCACAGA |
| | | | TGCGTAAGGAGAAAATACCGCATCAGGCGCCATTC |
| | | | GCCATTCAGGCTGCGCAACTGTTGGGAAGGGCGAT |
| | | | CGGTGCGGGCCTCTTCGCTATTACGCCAGCTGGCG |
| | | | AAAGGGGGATGTGCTGCAAGGCGATTAAGTTGGGT |
| | | | AACGCCAGGGTTTTCCCAGTCACGACGTTGTAAAA |
| | | | CGACGGCCAGTGAATTGACGCGTATTGggatGCCCG |
| | | | AGTGAGCACGCAGGGTCTCCATTTTGAAGCGGG |
| | | | AGGTTTGAACGCGCAGCCGCCATGccggggttttacgaga |
| | | | ttgtgattaaggtccccagcgaccttgacgagcatctgcccggcatttctgacagct |
| | | | ttgtgaactgggtggccgagaaggaatgggagttgccgccagattctgacatgga |
| | | | tctgaatctgattgagcaggcacccctgaccgtggccgagaagctgcagcgcga |
| | | | ctttctgacggaatggcgccgtgtgagtaaggccccggaggctcttttctttgtgca |
| | | | atttgagaagggagagagctacttccacatgcacgtgctcgtggaaaccaccgg |
| | | | ggTGAAATCCATGGTTTTGGGACGTTTCCTGAGTCAGAT |
| | | | TCGCGAAAAACTGATTCAGAGAATTTACCGCGGGATCG |
| | | | AGCCGACTTTGCCAAACTGGTTCGCGGTCACAAAGAC |
| | | | CAGAAATGGCGCCGGAGGCGGGAACAAGGTGGTGGA |
| | | | TGAGTGCTACATCCCCAATTACTTGCTCCCCAAAACCC |
| | | | AGCCTGAGCTCCAGTGGGCGTGGACTAATATGGAACA |
| | | | GTATTTAAGCGCCTGTTTGAATCTCACGGAGCGTAAAC |
| | | | GGTTGGTGGCGCAGCATCTGACGCACGTGTCGCAGAC |
| | | | GCAGGAGCAGAACAAAGAGAATCAGAATCCCAATTCT |
| | | | GATGCGCCGGTGATCAGATCAAAAACTTCAGCCAGGT |
| | | | ACATGGAGCTGGTCGGGTGGCTCGTGGACAAGGGGA |
| | | | TTACCTCGGAGAAGCAGTGGATCCAGGAGGACCAGGC |
| | | | CTCATACATCTCCTTCAATGCGGCCTCCAACTCGCGGT |
| | | | CCCAAATCAAGGCTGCCTTGGACAATGCGGGAAAGAT |
| | | | TATGAGCCTGACTAAAACCGCCCCCGACTACCTGGTG |
| | | | GGCCAGCAGCCCGTGGAGGACATTTCCAGCAATCGGA |
| | | | TTTATAAAATTTTGGAACTAAACGGGTACGATCCCCAAT |
| | | | ATGCGGCTTCCGTCTTTCTGGGATGGGCCACGAAAAA |
| | | | GTTCGGCAAGAGGAACACCATCTGGCTGTTTGGGCCT |
| | | | GCAACTACCGGGAAGACCAACATCGCGGAGGCCATAG |
| | | | CCCACACTGTGCCCTTCTACGGGTGCGTAAACTGGAC |
| | | | CAATGAGAACTTTCCCTTCAACGACTGTGTCGACAAGA |
| | | | TGGTGATCTGGTGGGAGGAGGGGAAGATGACCGCCA |
| | | | AGGTCGTGGAGTCGGCCAAAGCCATTCTCGGAGGAAG |
| | | | CAAGGTGCGCGTGGACCAGAAATGCAAGTCCTCGGCC |
| | | | CAGATAGACCCGACTCCCGTGATCGTCACCTCCAACA |
| | | | CCAACATGTGCGCCGTGATTGACGGGAACTCAACGAC |
| | | | CTTCGAACACCAGCAGCCGTTGCAAGACCGGATGTTC |
| | | | AAATTTGAACTCACCCGCCGTCTGGATCATGACTTTGG |
| | | | GAAGGTCACCAAGCAGGAAGTCAAAGACTTTTTCCGGT |
| | | | GGGCAAAGGATCACGTGGTTGAGGTGGAGCATGAATT |
| | | | CTACGTCAAAAAGGGTGGAGCCAAGAAAAGACCCGCC |
| | | | CCCAGTGACGCAGATATAAGTGAGCCCAAACGGGTGC |
| | | | GCGAGTCAGTTGCGCAGCCATCGACGTCAGACGCGG |
| | | | AAGCTTCGATCAACTACGCAGACAGGTACCAAAACAAA |
| | | | TGTTCTCGTCACGTGGGCATGAATCTGATGCTGTTTCC |
| | | | CTGCAGACAATGCGAGAGAATGAATCAGAATTCAAATA |
| | | | TCTGCTTCACTCACGGACAGAAAGACTGTTTAGAGTGC |
| | | | TTTCCCGTGTCAGAATCTCAACCCGTTTCTGTCGTCAA |
| | | | AAAGGCGTATCAGAAACTGTGCTACATTCATCATATCAT |
| | | | GGGAAAGGTGCCAGACGCTTGCACTGCCTGCGATCTG |
| | | | GTCAATGTGGATTTGGATGACTGCATCTTTGAACAATA |
| | | | AATGATTTAAATCAGGTATGGCTGCCGATGGTTATC |
| | | | TTCCAGATTGGCTCGAGGACAACCTTAGTGAAGGA |
| | | | ATTCGCGAGTGGTGGGCTTTGAAACCTGGAGCCCC |
| | | | TCAACCCAAGGCAAATCAACAACATCAAGACAACG |
| | | | CTCGAGGTCTTGTGCTTCCGGGTTACAAATACCTTG |
| | | | GACCCGGCAACGGACTCGACAAGGGGGAGCCGGT |
| | | | CAACGCAGCAGACGCGGCGGCCCTCGAGCACGAC |
| | | | AAGGCCTACGACCAGCAGCTCAAGGCCGGAGACA |
| | | | ACCCGTACCTCAAGTACAACCACGCCGACGCCGAG |
| | | | TTCCAGGAGCGGCTCAAAGAAGATACGTCTTTTGG |
| | | | GGGCAACCTCGGGCGAGCAGTCTTCCAGGCCAAAA |
| | | | AGAGGCTTCTTGAACCTCTTGGTCTGGTTGAGGAA |
| | | | GCGGCTAAGACGGCTCCTGGAAAGAAGAGGCCTGT |
| | | | AGAGCAGTCTCCTCAGGAACCGGACTCCTCCGCGG |
| | | | GTATTGGCAAATCGGGTGCACAGCCCGCTAAAAAG |
| | | | AGACTCAATTTCGGTCAGACTGGCGACACAGAGTC |
| | | | AGTCCCAGACCCTCAACCAATCGGAGAACCTCCCG |
| | | | CAGCCCCCTCAGGTGTGGGATCTCTTACAATGGCTT |
| | | | CAGGTGGTGGCGCACCAGTGGCAGACAATAACGA |
| | | | AGGTGCCGATGGAGTGGGTAGTTCCTCGGGAAATT |
| | | | GGCATTGCGATTCCCAATGGCTGGGGGACAGAGTC |
| | | | ATCACCACCAGCACCCGAACCTGGGCCCTGCCCAC |
| | | | CTACAACAATCACCTCTACAAGCAAATCTCCAACA |
| | | | GCACATCTGGAGGATCTTCAAATGACAACGCCTAC |
| | | | TTCGGCTACAGCACCCCCTGGGGGTATTTTGACTTC |
| | | | AACAGATTCCACTGCCACTTCTCACCACGTGACTG |
| | | | GCAGCGACTCATCAACAACAACTGGGGATTCCGGC |
| | | | CTAAGCGACTCAACTTCAAGCTCTTCAACATTCAG |
| | | | GTCAAAGAGGTTACGGACAACAATGGAGTCAAGA |
| | | | CCATCGCCAATAACCTTACCAGCACGGTCCAGGTC |
| | | | TTCACGGACTCAGACTATCAGCTCCCGTACGTGCTC |
| | | | GGGTCGGCTCACGAGGGCTGCCTCCCGCCGTTCCC |
| | | | AGCGGACGTTTTCATGATTCCTCAGTACGGGTATCT |
| | | | GACGCTTAATGATGGAAGCCAGGCCGTGGGTCGTT |
| | | | CGTCCTTTTACTGCCTGGAATATTTCCCGTCGCAAA |
| | | | TGCTAAGAACGGGTAACAACTTCCAGTTCAGCTAC |
| | | | GAGTTTGAGAACGTACCTTTCCATAGCAGCTACGC |
| | | | TCACAGCCAAAGCCTGGACCGACTAATGAATCCAC |
| | | | TCATCGACCAATACTTGTACTATCTCTCAAAGACTA |
| | | | TTAACGGTTCTGGACAGAATCAACAAACGCTAAAA |
| | | | TTCAGTGTGGCCGGACCCAGCAACATGGCTGTCCA |
| | | | GGGAAGAAACTACATACCTGGACCCAGCTACCGAC |
| | | | AACAACGTGTCTCAACCACTGTGACTCAAAACAAC |
| | | | AACAGCGAATTTGCTTGGCCTGGAGCTTCTTCTTGG |
| | | | GCTCTCAATGGACGTAATAGCTTGATGAATCCTGG |
| | | | ACCTGCTATGGCCAGCCACAAAGAAGGAGAGGAC |
| | | | CGTTTCTTTCCTTTGTCTGGATCTTTAATTTTTGGCA |
| | | | AACAAGGAACTGGAAGAGACAACGTGGATGCGGA |
| | | | CAAAGTCATGATAACCAACGAAGAAGAAATTAAA |
| | | | ACTACTAACCCGGTAGCAACGGAGTCCTATGGACA |
| | | | AGTGGCCACAAACCACCAGAGTGCCCAAGCACAG |
| | | | GCGCAGACCGGCTGGGTTCAAAACCAAGGAATACT |
| | | | TCCGGGTATGGTTTGGCAGGACAGAGATGTGTACC |
| | | | TGCAAGGACCCATTTGGGCCAAAATTCCTCACACG |
| | | | GACGGCAACTTTCACCCTTCTCCGCTGATGGGAGG |
| | | | GTTTGGAATGAAGCACCCGCCTCCTCAGATCCTCAT |
| | | | CAAAAACACACCTGTACCTGCGGATCCTCCAACGG |
| | | | CCTTCAACAAGGACAAGCTGAACTCTTTCATCACC |
| | | | CAGTATTCTACTGGCCAAGTCAGCGTGGAGATCGA |
| | | | GTGGGAGCTGCAGAAGGAAAACAGCAAGCGCTGG |
| | | | AACCCGGAGATCCAGTACACTTCCAACTATTACAA |
| | | | GTCTAATAATGTTGAATTTGCTGTTAATACTGAAGG |
| | | | TGTATATAGTGAACCCCGCCCCATTGGCACCAGAT |
| | | | ACCTGACTCGTAATCTGTAATTGCTTGTTAATCAA |
| | | | TAAACCGTTTAATTCGTTTCAGTTGAACTTTGGTC |
| | | | TCTGCGGAGGTCCTGTATTAGAGGTCACGTGAGT |
| | | | GTTTTGCGACATTTTGCGACACCATGTGGTCAC |
| | | | GCTGGGTATTTAAGCCCGAGTGAGCACGCAGGG |
| | | | TCTCCATTTTGAAGCGGGAGGTTTGAAATCCCAA |
| | | | TGGCGCGCCGAGCTTGGCTCGAGCATGGTCATAGC |
| | | | TGTTTCCTGTGTGAAATTGTTATCCGCTCACAATTC |
| | | | CACACAACATACGAGCCGGAAGCATAAAGTGTAA |
| | | | AGCCTGGGGTGCCTAATGAGTGAGCTAACTCACAT |
| | | | TAATTGCGTTGCGCTCACTGCCCGCTTTCCAGTCGG |
| | | | GAAACCTGTCGTGCCAGCTGCATTAATGAATCGGC |
| | | | CAACGCGCGGGGAGAGGCGGTTTGCGTATTGGGCG |
| | | | CTCTTCCGCTTCCTCGCTCACTGACTCGCTGCGCTC |
| | | | GGTCGTTCGGCTGCGGCGAGCGGTATCAGCTCACT |
| | | | CAAAGGCGGTAATACGGTTATCCACAGAATCAGGG |
| | | | GATAACGCAGGAAAGAACATGTGAGCAAAAGGCC |
| | | | AGCAAAAGGCCAGGAACCGTAAAAAGGCCGCGTT |
| | | | GCTGGCGTTTTTCCATAGGCTCCGCCCCCCTGACGA |
| | | | GCATCACAAAAATCGACGCTCAAGTCAGAGGTGGC |
| | | | GAAACCCGACAGGACTATAAAGATACCAGGCGTTT |
| | | | CCCCCTGGAAGCTCCCTCGTGCGCTCTCCTGTTCCG |
| | | | ACCCTGCCGCTTACCGGATACCTGTCCGCCTTTCTC |
| | | | CCTTCGGGAAGCGTGGCGCTTTCTCATAGCTCACGC |
| | | | TGTAGGTATCTCAGTTCGGTGTAGGTCGTTCGCTCC |
| | | | AAGCTGGGCTGTGTGCACGAACCCCCCGTTCAGCC |
| | | | CGACCGCTGCGCCTTATCCGGTAACTATCGTCTTGA |
| | | | GTCCAACCCGGTAAGACACGACTTATCGCCACTGG |
| | | | CAGCAGCCACTGGTAACAGGATTAGCAGAGCGAG |
| | | | GTATGTAGGCGGTGCTACAGAGTTCTTGAAGTGGT |
| | | | GGCCTAACTACGGCTACACTAGAAGAACAGTATTT |
| | | | GGTATCTGCGCTCTGCTGAAGCCAGTTACCTTCGGA |
| | | | AAAAGAGTTGGTAGCTCTTGATCCGGCAAACAAAC |
| | | | CACCGCTGGTAGCGGTGGTTTTTTTGTTTGCAAGCA |
| | | | GCAGATTACGCGCAGAAAAAAAGGATCTCAAGAA |
| | | | GATCCTTTGATCTTTTCTACGGGGTCTGACGCTCAG |
| | | | TGGAACGAAAACTCACGTTAAGGGATTTTGGTCAT |
| | | | GAGATTATCAAAAAGGATCTTCACCTAGATCCTTTT |
| | | | AAATTAAAAATGAAGTTTTAAATCAATCTAAAGTA |
| | | | TATATGAGTAAACTTGGTCTGACAGTTAGAAAAAC |
| | | | TCATCGAGCATCAAATGAAACTGCAATTTATTCAT |
| | | | ATCAGGATTATCAATACCATATTTTTGAAAAAGCC |
| | | | GTTTCTGTAATGAAGGAGAAAACTCACCGAGGCAG |
| | | | TTCCATAGGATGGCAAGATCCTGGTATCGGTCTGC |
| | | | GATTCCGACTCGTCCAACATCAATACAACCTATTA |
| | | | ATTTCCCCTCGTCAAAAATAAGGTTATCAAGTGAG |
| | | | AAATCACCATGAGTGACGACTGAATCCGGTGAGAA |
| | | | TGGCAAAAGTTTATGCATTTCTTTCCAGACTTGTTC |
| | | | AACAGGCCAGCCATTACGCTCGTCATCAAAATCAC |
| | | | TCGCATCAACCAAACCGTTATTCATTCGTGATTGCG |
| | | | CCTGAGCGAGACGAAATACGCGATCGCTGTTAAAA |
| | | | GGACAATTACAAACAGGAATCGAATGCAACCGGC |
| | | | GCAGGAACACTGCCAGCGCATCAACAATATTTTCA |
| | | | CCTGAATCAGGATATTCTTCTAATACCTGGAATGCT |
| | | | GTTTTCCCAGGGATCGCAGTGGTGAGTAACCATGC |
| | | | ATCATCAGGAGTACGGATAAAATGCTTGATGGTCG |
| | | | GAAGAGGCATAAATTCCGTCAGCCAGTTTAGTCTG |
| | | | ACCATCTCATCTGTAACATCATTGGCAACGCTACCT |
| | | | TTGCCATGTTTCAGAAACAACTCTGGCGCATCGGG |
| | | | CTTCCCATACAATCGATAGATTGTCGCACCTGATTG |
| | | | CCCGACATTATCGCGAGCCCATTTATACCCATATAA |
| | | | ATCAGCATCCATGTTGGAATTTAATCGCGGCCTAG |
| | | | AGCAAGACGTTTCCCGTTGAATATGGCTCATACTCT |
| | | | TCCTTTTTCAATATTATTGAAGCATTTATCAGGGTT |
| | | | ATTGTCTCATGAGCGGATACATATTTGAATGTATTT |
| | | | AGAAAAATAAACAAATAGGGGTTCCGCGCACATTT |
| | | | CCCCGAAAAGTGCCACCTGACGTCTAAGAAACCAT |
| | | | TATTATCATGACATTAACCTATAAAAATAGGCGTA |
| | | | TCACGAGGCCCTTTCGTC |
| 39 | pYZZ281 | p5v4-Rep2Ca9- | TCGCGCGTTTCGGTGATGACGGTGAAAACCTCTGA |
| | | P5 | CACATGCAGCTCCCGGAGACGGTCACAGCTTGTCT |
| | | | GTAAGCGGATGCCGGGAGCAGACAAGCCCGTCAG |
| | | | GGCGCGTCAGCGGGTGTTGGCGGGTGTCGGGGCTG |
| | | | GCTTAACTATGCGGCATCAGAGCAGATTGTACTGA |
| | | | GAGTGCACCATATGCGGTGTGAAATACCGCACAGA |
| | | | TGCGTAAGGAGAAAATACCGCATCAGGCGCCATTC |
| | | | GCCATTCAGGCTGCGCAACTGTTGGGAAGGGCGAT |
| | | | CGGTGCGGGCCTCTTCGCTATTACGCCAGCTGGCG |
| | | | AAAGGGGGATGTGCTGCAAGGCGATTAAGTTGGGT |
| | | | AACGCCAGGGTTTTCCCAGTCACGACGTTGTAAAA |
| | | | CGACGGCCAGTGAATTGACGCGTATTGggatTATTTA |
| | | | AGCCCGAGTGAGCACGCAGGGTCTCCATTTTGA |
| | | | AGCGGGAGGTTTGAACGCGCAGCCGCCATGccggg |
| | | | gttttacgagattgtgattaaggtccccagcgaccttgacgagcatctgcccggcat |
| | | | ttctgacagctttgtgaactgggtggccgagaaggaatgggagttgccgccagatt |
| | | | ctgacatggatctgaatctgattgagcaggcacccctgaccgtggccgagaagct |
| | | | gcagcgcgactttctgacggaatggcgccgtgtgagtaaggccccggaggctctt |
| | | | ttctttgtgcaatttgagaagggagagagctacttccacatgcacgtgctcgtggaa |
| | | | accaccggggTGAAATCCATGGTTTTGGGACGTTTCCTGA |
| | | | GTCAGATTCGCGAAAAACTGATTCAGAGAATTTACCGC |
| | | | GGGATCGAGCCGACTTTGCCAAACTGGTTCGCGGTCA |
| | | | CAAAGACCAGAAATGGCGCCGGAGGCGGGAACAAGG |
| | | | TGGTGGATGAGTGCTACATCCCCAATTACTTGCTCCCC |
| | | | AAAACCCAGCCTGAGCTCCAGTGGGCGTGGACTAATA |
| | | | TGGAACAGTATTTAAGCGCCTGTTTGAATCTCACGGAG |
| | | | CGTAAACGGTTGGTGGCGCAGCATCTGACGCACGTGT |
| | | | CGCAGACGCAGGAGCAGAACAAAGAGAATCAGAATCC |
| | | | CAATTCTGATGCGCCGGTGATCAGATCAAAAACTTCAG |
| | | | CCAGGTACATGGAGCTGGTCGGGTGGCTCGTGGACAA |
| | | | GGGGATTACCTCGGAGAAGCAGTGGATCCAGGAGGA |
| | | | CCAGGCCTCATACATCTCCTTCAATGCGGCCTCCAACT |
| | | | CGCGGTCCCAAATCAAGGCTGCCTTGGACAATGCGGG |
| | | | AAAGATTATGAGCCTGACTAAAACCGCCCCCGACTACC |
| | | | TGGTGGGCCAGCAGCCCGTGGAGGACATTTCCAGCAA |
| | | | TCGGATTTATAAAATTTTGGAACTAAACGGGTACGATC |
| | | | CCCAATATGCGGCTTCCGTCTTTCTGGGATGGGCCAC |
| | | | GAAAAAGTTCGGCAAGAGGAACACCATCTGGCTGTTT |
| | | | GGGCCTGCAACTACCGGGAAGACCAACATCGCGGAG |
| | | | GCCATAGCCCACACTGTGCCCTTCTACGGGTGCGTAA |
| | | | ACTGGACCAATGAGAACTTTCCCTTCAACGACTGTGTC |
| | | | GACAAGATGGTGATCTGGTGGGAGGAGGGGAAGATG |
| | | | ACCGCCAAGGTCGTGGAGTCGGCCAAAGCCATTCTCG |
| | | | GAGGAAGCAAGGTGCGCGTGGACCAGAAATGCAAGTC |
| | | | CTCGGCCCAGATAGACCCGACTCCCGTGATCGTCACC |
| | | | TCCAACACCAACATGTGCGCCGTGATTGACGGGAACT |
| | | | CAACGACCTTCGAACACCAGCAGCCGTTGCAAGACCG |
| | | | GATGTTCAAATTTGAACTCACCCGCCGTCTGGATCATG |
| | | | ACTTTGGGAAGGTCACCAAGCAGGAAGTCAAAGACTTT |
| | | | TTCCGGTGGGCAAAGGATCACGTGGTTGAGGTGGAGC |
| | | | ATGAATTCTACGTCAAAAAGGGTGGAGCCAAGAAAAGA |
| | | | CCCGCCCCCAGTGACGCAGATATAAGTGAGCCCAAAC |
| | | | GGGTGCGCGAGTCAGTTGCGCAGCCATCGACGTCAG |
| | | | ACGCGGAAGCTTCGATCAACTACGCAGACAGGTACCA |
| | | | AAACAAATGTTCTCGTCACGTGGGCATGAATCTGATGC |
| | | | TGTTTCCCTGCAGACAATGCGAGAGAATGAATCAGAAT |
| | | | TCAAATATCTGCTTCACTCACGGACAGAAAGACTGTTT |
| | | | AGAGTGCTTTCCCGTGTCAGAATCTCAACCCGTTTCTG |
| | | | TCGTCAAAAAGGCGTATCAGAAACTGTGCTACATTCAT |
| | | | CATATCATGGGAAAGGTGCCAGACGCTTGCACTGCCT |
| | | | GCGATCTGGTCAATGTGGATTTGGATGACTGCATCTTT |
| | | | GAACAATAAATGATTTAAATCAGGTATGGCTGCCGA |
| | | | TGGTTATCTTCCAGATTGGCTCGAGGACAACCTTAG |
| | | | TGAAGGAATTCGCGAGTGGTGGGCTTTGAAACCTG |
| | | | GAGCCCCTCAACCCAAGGCAAATCAACAACATCAA |
| | | | GACAACGCTCGAGGTCTTGTGCTTCCGGGTTACAA |
| | | | ATACCTTGGACCCGGCAACGGACTCGACAAGGGGG |
| | | | AGCCGGTCAACGCAGCAGACGCGGCGGCCCTCGAG |
| | | | CACGACAAGGCCTACGACCAGCAGCTCAAGGCCGG |
| | | | AGACAACCCGTACCTCAAGTACAACCACGCCGACG |
| | | | CCGAGTTCCAGGAGCGGCTCAAAGAAGATACGTCT |
| | | | TTTGGGGGCAACCTCGGGCGAGCAGTCTTCCAGGC |
| | | | CAAAAAGAGGCTTCTTGAACCTCTTGGTCTGGTTG |
| | | | AGGAAGCGGCTAAGACGGCTCCTGGAAAGAAGAG |
| | | | GCCTGTAGAGCAGTCTCCTCAGGAACCGGACTCCT |
| | | | CCGCGGGTATTGGCAAATCGGGTGCACAGCCCGCT |
| | | | AAAAAGAGACTCAATTTCGGTCAGACTGGCGACAC |
| | | | AGAGTCAGTCCCAGACCCTCAACCAATCGGAGAAC |
| | | | CTCCCGCAGCCCCCTCAGGTGTGGGATCTCTTACAA |
| | | | TGGCTTCAGGTGGTGGCGCACCAGTGGCAGACAAT |
| | | | AACGAAGGTGCCGATGGAGTGGGTAGTTCCTCGGG |
| | | | AAATTGGCATTGCGATTCCCAATGGCTGGGGGACA |
| | | | GAGTCATCACCACCAGCACCCGAACCTGGGCCCTG |
| | | | CCCACCTACAACAATCACCTCTACAAGCAAATCTC |
| | | | CAACAGCACATCTGGAGGATCTTCAAATGACAACG |
| | | | CCTACTTCGGCTACAGCACCCCCTGGGGGTATTTTG |
| | | | ACTTCAACAGATTCCACTGCCACTTCTCACCACGTG |
| | | | ACTGGCAGCGACTCATCAACAACAACTGGGGATTC |
| | | | CGGCCTAAGCGACTCAACTTCAAGCTCTTCAACATT |
| | | | CAGGTCAAAGAGGTTACGGACAACAATGGAGTCA |
| | | | AGACCATCGCCAATAACCTTACCAGCACGGTCCAG |
| | | | GTCTTCACGGACTCAGACTATCAGCTCCCGTACGTG |
| | | | CTCGGGTCGGCTCACGAGGGCTGCCTCCCGCCGTT |
| | | | CCCAGCGGACGTTTTCATGATTCCTCAGTACGGGTA |
| | | | TCTGACGCTTAATGATGGAAGCCAGGCCGTGGGTC |
| | | | GTTCGTCCTTTTACTGCCTGGAATATTTCCCGTCGC |
| | | | AAATGCTAAGAACGGGTAACAACTTCCAGTTCAGC |
| | | | TACGAGTTTGAGAACGTACCTTTCCATAGCAGCTA |
| | | | CGCTCACAGCCAAAGCCTGGACCGACTAATGAATC |
| | | | CACTCATCGACCAATACTTGTACTATCTCTCAAAGA |
| | | | CTATTAACGGTTCTGGACAGAATCAACAAACGCTA |
| | | | AAATTCAGTGTGGCCGGACCCAGCAACATGGCTGT |
| | | | CCAGGGAAGAAACTACATACCTGGACCCAGCTACC |
| | | | GACAACAACGTGTCTCAACCACTGTGACTCAAAAC |
| | | | AACAACAGCGAATTTGCTTGGCCTGGAGCTTCTTCT |
| | | | TGGGCTCTCAATGGACGTAATAGCTTGATGAATCC |
| | | | TGGACCTGCTATGGCCAGCCACAAAGAAGGAGAG |
| | | | GACCGTTTCTTTCCTTTGTCTGGATCTTTAATTTTTG |
| | | | GCAAACAAGGAACTGGAAGAGACAACGTGGATGC |
| | | | GGACAAAGTCATGATAACCAACGAAGAAGAAATT |
| | | | AAAACTACTAACCCGGTAGCAACGGAGTCCTATGG |
| | | | ACAAGTGGCCACAAACCACCAGAGTGCCCAAGCAC |
| | | | AGGCGCAGACCGGCTGGGTTCAAAACCAAGGAAT |
| | | | ACTTCCGGGTATGGTTTGGCAGGACAGAGATGTGT |
| | | | ACCTGCAAGGACCCATTTGGGCCAAAATTCCTCAC |
| | | | ACGGACGGCAACTTTCACCCTTCTCCGCTGATGGG |
| | | | AGGGTTTGGAATGAAGCACCCGCCTCCTCAGATCC |
| | | | TCATCAAAAACACACCTGTACCTGCGGATCCTCCA |
| | | | ACGGCCTTCAACAAGGACAAGCTGAACTCTTTCAT |
| | | | CACCCAGTATTCTACTGGCCAAGTCAGCGTGGAGA |
| | | | TCGAGTGGGAGCTGCAGAAGGAAAACAGCAAGCG |
| | | | CTGGAACCCGGAGATCCAGTACACTTCCAACTATT |
| | | | ACAAGTCTAATAATGTTGAATTTGCTGTTAATACTG |
| | | | AAGGTGTATATAGTGAACCCCGCCCCATTGGCACC |
| | | | AGATACCTGACTCGTAATCTGTAATTGCTTGTTAA |
| | | | TCAATAAACCGTTTAATTCGTTTCAGTTGAACTT |
| | | | TGGTCTCTGCGGAGGTCCTGTATTAGAGGTCACG |
| | | | TGAGTGTTTTGCGACATTTTGCGACACCATGTG |
| | | | GTCACGCTGGGTATTTAAGCCCGAGTGAGCACG |
| | | | CAGGGTCTCCATTTTGAAGCGGGAGGTTTGAAA |
| | | | TCCCAATGGCGCGCCGAGCTTGGCTCGAGCATGGT |
| | | | CATAGCTGTTTCCTGTGTGAAATTGTTATCCGCTCA |
| | | | CAATTCCACACAACATACGAGCCGGAAGCATAAAG |
| | | | TGTAAAGCCTGGGGTGCCTAATGAGTGAGCTAACT |
| | | | CACATTAATTGCGTTGCGCTCACTGCCCGCTTTCCA |
| | | | GTCGGGAAACCTGTCGTGCCAGCTGCATTAATGAA |
| | | | TCGGCCAACGCGCGGGGAGAGGCGGTTTGCGTATT |
| | | | GGGCGCTCTTCCGCTTCCTCGCTCACTGACTCGCTG |
| | | | CGCTCGGTCGTTCGGCTGCGGCGAGCGGTATCAGC |
| | | | TCACTCAAAGGCGGTAATACGGTTATCCACAGAAT |
| | | | CAGGGGATAACGCAGGAAAGAACATGTGAGCAAA |
| | | | AGGCCAGCAAAAGGCCAGGAACCGTAAAAAGGCC |
| | | | GCGTTGCTGGCGTTTTTCCATAGGCTCCGCCCCCCT |
| | | | GACGAGCATCACAAAAATCGACGCTCAAGTCAGAG |
| | | | GTGGCGAAACCCGACAGGACTATAAAGATACCAG |
| | | | GCGTTTCCCCCTGGAAGCTCCCTCGTGCGCTCTCCT |
| | | | GTTCCGACCCTGCCGCTTACCGGATACCTGTCCGCC |
| | | | TTTCTCCCTTCGGGAAGCGTGGCGCTTTCTCATAGC |
| | | | TCACGCTGTAGGTATCTCAGTTCGGTGTAGGTCGTT |
| | | | CGCTCCAAGCTGGGCTGTGTGCACGAACCCCCCGT |
| | | | TCAGCCCGACCGCTGCGCCTTATCCGGTAACTATCG |
| | | | TCTTGAGTCCAACCCGGTAAGACACGACTTATCGC |
| | | | CACTGGCAGCAGCCACTGGTAACAGGATTAGCAGA |
| | | | GCGAGGTATGTAGGCGGTGCTACAGAGTTCTTGAA |
| | | | GTGGTGGCCTAACTACGGCTACACTAGAAGAACAG |
| | | | TATTTGGTATCTGCGCTCTGCTGAAGCCAGTTACCT |
| | | | TCGGAAAAAGAGTTGGTAGCTCTTGATCCGGCAAA |
| | | | CAAACCACCGCTGGTAGCGGTGGTTTTTTTGTTTGC |
| | | | AAGCAGCAGATTACGCGCAGAAAAAAAGGATCTC |
| | | | AAGAAGATCCTTTGATCTTTTCTACGGGGTCTGACG |
| | | | CTCAGTGGAACGAAAACTCACGTTAAGGGATTTTG |
| | | | GTCATGAGATTATCAAAAAGGATCTTCACCTAGAT |
| | | | CCTTTTAAATTAAAAATGAAGTTTTAAATCAATCTA |
| | | | AAGTATATATGAGTAAACTTGGTCTGACAGTTAGA |
| | | | AAAACTCATCGAGCATCAAATGAAACTGCAATTTA |
| | | | TTCATATCAGGATTATCAATACCATATTTTTGAAAA |
| | | | AGCCGTTTCTGTAATGAAGGAGAAAACTCACCGAG |
| | | | GCAGTTCCATAGGATGGCAAGATCCTGGTATCGGT |
| | | | CTGCGATTCCGACTCGTCCAACATCAATACAACCT |
| | | | ATTAATTTCCCCTCGTCAAAAATAAGGTTATCAAGT |
| | | | GAGAAATCACCATGAGTGACGACTGAATCCGGTGA |
| | | | GAATGGCAAAAGTTTATGCATTTCTTTCCAGACTTG |
| | | | TTCAACAGGCCAGCCATTACGCTCGTCATCAAAAT |
| | | | CACTCGCATCAACCAAACCGTTATTCATTCGTGATT |
| | | | GCGCCTGAGCGAGACGAAATACGCGATCGCTGTTA |
| | | | AAAGGACAATTACAAACAGGAATCGAATGCAACC |
| | | | GGCGCAGGAACACTGCCAGCGCATCAACAATATTT |
| | | | TCACCTGAATCAGGATATTCTTCTAATACCTGGAAT |
| | | | GCTGTTTTCCCAGGGATCGCAGTGGTGAGTAACCA |
| | | | TGCATCATCAGGAGTACGGATAAAATGCTTGATGG |
| | | | TCGGAAGAGGCATAAATTCCGTCAGCCAGTTTAGT |
| | | | CTGACCATCTCATCTGTAACATCATTGGCAACGCTA |
| | | | CCTTTGCCATGTTTCAGAAACAACTCTGGCGCATCG |
| | | | GGCTTCCCATACAATCGATAGATTGTCGCACCTGAT |
| | | | TGCCCGACATTATCGCGAGCCCATTTATACCCATAT |
| | | | AAATCAGCATCCATGTTGGAATTTAATCGCGGCCT |
| | | | AGAGCAAGACGTTTCCCGTTGAATATGGCTCATAC |
| | | | TCTTCCTTTTTCAATATTATTGAAGCATTTATCAGG |
| | | | GTTATTGTCTCATGAGCGGATACATATTTGAATGTA |
| | | | TTTAGAAAAATAAACAAATAGGGGTTCCGCGCACA |
| | | | TTTCCCCGAAAAGTGCCACCTGACGTCTAAGAAAC |
| | | | CATTATTATCATGACATTAACCTATAAAAATAGGC |
| | | | GTATCACGAGGCCCTTTCGTC |
| 40 | pYZZ283 | p5v5-Rep2Ca9- | TCGCGCGTTTCGGTGATGACGGTGAAAACCTCTGA |
| | | P5 | CACATGCAGCTCCCGGAGACGGTCACAGCTTGTCT |
| | | | GTAAGCGGATGCCGGGAGCAGACAAGCCCGTCAG |
| | | | GGCGCGTCAGCGGGTGTTGGCGGGTGTCGGGGCTG |
| | | | GCTTAACTATGCGGCATCAGAGCAGATTGTACTGA |
| | | | GAGTGCACCATATGCGGTGTGAAATACCGCACAGA |
| | | | TGCGTAAGGAGAAAATACCGCATCAGGCGCCATTC |
| | | | GCCATTCAGGCTGCGCAACTGTTGGGAAGGGCGAT |
| | | | CGGTGCGGGCCTCTTCGCTATTACGCCAGCTGGCG |
| | | | AAAGGGGGATGTGCTGCAAGGCGATTAAGTTGGGT |
| | | | AACGCCAGGGTTTTCCCAGTCACGACGTTGTAAAA |
| | | | CGACGGCCAGTGAATTGACGCGTATTGggatGAGGT |
| | | | CCTGTATTAGATTTGCGACATTTTGCGACACCAT |
| | | | GTGGTCACGCTGGGTATTTAAGCCCGAGTGAGC |
| | | | ACGCAGGGTCTCCATTTTGAAGCGGGAGGTTTG |
| | | | AACGCGCAGCCGCCATGccggggttttacgagattgtgattaaggt |
| | | | ccccagcgaccttgacgagcatctgcccggcatttctgacagctttgtgaactgggt |
| | | | ggccgagaaggaatgggagttgccgccagattctgacatggatctgaatctgatt |
| | | | gagcaggcacccctgaccgtggccgagaagctgcagcgcgactttctgacgga |
| | | | atggcgccgtgtgagtaaggccccggaggctcttttctttgtgcaatttgagaaggg |
| | | | agagagctacttccacatgcacgtgctcgtggaaaccaccggggTGAAATC |
| | | | CATGGTTTTGGGACGTTTCCTGAGTCAGATTCGCGAAA |
| | | | AACTGATTCAGAGAATTTACCGCGGGATCGAGCCGAC |
| | | | TTTGCCAAACTGGTTCGCGGTCACAAAGACCAGAAATG |
| | | | GCGCCGGAGGCGGGAACAAGGTGGTGGATGAGTGCT |
| | | | ACATCCCCAATTACTTGCTCCCCAAAACCCAGCCTGAG |
| | | | CTCCAGTGGGCGTGGACTAATATGGAACAGTATTTAAG |
| | | | CGCCTGTTTGAATCTCACGGAGCGTAAACGGTTGGTG |
| | | | GCGCAGCATCTGACGCACGTGTCGCAGACGCAGGAG |
| | | | CAGAACAAAGAGAATCAGAATCCCAATTCTGATGCGCC |
| | | | GGTGATCAGATCAAAAACTTCAGCCAGGTACATGGAG |
| | | | CTGGTCGGGTGGCTCGTGGACAAGGGGATTACCTCG |
| | | | GAGAAGCAGTGGATCCAGGAGGACCAGGCCTCATACA |
| | | | TCTCCTTCAATGCGGCCTCCAACTCGCGGTCCCAAATC |
| | | | AAGGCTGCCTTGGACAATGCGGGAAAGATTATGAGCC |
| | | | TGACTAAAACCGCCCCCGACTACCTGGTGGGCCAGCA |
| | | | GCCCGTGGAGGACATTTCCAGCAATCGGATTTATAAAA |
| | | | TTTTGGAACTAAACGGGTACGATCCCCAATATGCGGCT |
| | | | TCCGTCTTTCTGGGATGGGCCACGAAAAAGTTCGGCA |
| | | | AGAGGAACACCATCTGGCTGTTTGGGCCTGCAACTAC |
| | | | CGGGAAGACCAACATCGCGGAGGCCATAGCCCACACT |
| | | | GTGCCCTTCTACGGGTGCGTAAACTGGACCAATGAGA |
| | | | ACTTTCCCTTCAACGACTGTGTCGACAAGATGGTGATC |
| | | | TGGTGGGAGGAGGGGAAGATGACCGCCAAGGTCGTG |
| | | | GAGTCGGCCAAAGCCATTCTCGGAGGAAGCAAGGTGC |
| | | | GCGTGGACCAGAAATGCAAGTCCTCGGCCCAGATAGA |
| | | | CCCGACTCCCGTGATCGTCACCTCCAACACCAACATGT |
| | | | GCGCCGTGATTGACGGGAACTCAACGACCTTCGAACA |
| | | | CCAGCAGCCGTTGCAAGACCGGATGTTCAAATTTGAAC |
| | | | TCACCCGCCGTCTGGATCATGACTTTGGGAAGGTCAC |
| | | | CAAGCAGGAAGTCAAAGACTTTTTCCGGTGGGCAAAG |
| | | | GATCACGTGGTTGAGGTGGAGCATGAATTCTACGTCAA |
| | | | AAAGGGTGGAGCCAAGAAAAGACCCGCCCCCAGTGAC |
| | | | GCAGATATAAGTGAGCCCAAACGGGTGCGCGAGTCAG |
| | | | TTGCGCAGCCATCGACGTCAGACGCGGAAGCTTCGAT |
| | | | CAACTACGCAGACAGGTACCAAAACAAATGTTCTCGTC |
| | | | ACGTGGGCATGAATCTGATGCTGTTTCCCTGCAGACAA |
| | | | TGCGAGAGAATGAATCAGAATTCAAATATCTGCTTCAC |
| | | | TCACGGACAGAAAGACTGTTTAGAGTGCTTTCCCGTGT |
| | | | CAGAATCTCAACCCGTTTCTGTCGTCAAAAAGGCGTAT |
| | | | CAGAAACTGTGCTACATTCATCATATCATGGGAAAGGT |
| | | | GCCAGACGCTTGCACTGCCTGCGATCTGGTCAATGTG |
| | | | GATTTGGATGACTGCATCTTTGAACAATAAATGATTTA |
| | | | AATCAGGTATGGCTGCCGATGGTTATCTTCCAGATT |
| | | | GGCTCGAGGACAACCTTAGTGAAGGAATTCGCGAG |
| | | | TGGTGGGCTTTGAAACCTGGAGCCCCTCAACCCAA |
| | | | GGCAAATCAACAACATCAAGACAACGCTCGAGGTC |
| | | | TTGTGCTTCCGGGTTACAAATACCTTGGACCCGGCA |
| | | | ACGGACTCGACAAGGGGGAGCCGGTCAACGCAGC |
| | | | AGACGCGGCGGCCCTCGAGCACGACAAGGCCTACG |
| | | | ACCAGCAGCTCAAGGCCGGAGACAACCCGTACCTC |
| | | | AAGTACAACCACGCCGACGCCGAGTTCCAGGAGCG |
| | | | GCTCAAAGAAGATACGTCTTTTGGGGGCAACCTCG |
| | | | GGCGAGCAGTCTTCCAGGCCAAAAAGAGGCTTCTT |
| | | | GAACCTCTTGGTCTGGTTGAGGAAGCGGCTAAGAC |
| | | | GGCTCCTGGAAAGAAGAGGCCTGTAGAGCAGTCTC |
| | | | CTCAGGAACCGGACTCCTCCGCGGGTATTGGCAAA |
| | | | TCGGGTGCACAGCCCGCTAAAAAGAGACTCAATTT |
| | | | CGGTCAGACTGGCGACACAGAGTCAGTCCCAGACC |
| | | | CTCAACCAATCGGAGAACCTCCCGCAGCCCCCTCA |
| | | | GGTGTGGGATCTCTTACAATGGCTTCAGGTGGTGG |
| | | | CGCACCAGTGGCAGACAATAACGAAGGTGCCGATG |
| | | | GAGTGGGTAGTTCCTCGGGAAATTGGCATTGCGAT |
| | | | TCCCAATGGCTGGGGGACAGAGTCATCACCACCAG |
| | | | CACCCGAACCTGGGCCCTGCCCACCTACAACAATC |
| | | | ACCTCTACAAGCAAATCTCCAACAGCACATCTGGA |
| | | | GGATCTTCAAATGACAACGCCTACTTCGGCTACAG |
| | | | CACCCCCTGGGGGTATTTTGACTTCAACAGATTCCA |
| | | | CTGCCACTTCTCACCACGTGACTGGCAGCGACTCAT |
| | | | CAACAACAACTGGGGATTCCGGCCTAAGCGACTCA |
| | | | ACTTCAAGCTCTTCAACATTCAGGTCAAAGAGGTT |
| | | | ACGGACAACAATGGAGTCAAGACCATCGCCAATAA |
| | | | CCTTACCAGCACGGTCCAGGTCTTCACGGACTCAG |
| | | | ACTATCAGCTCCCGTACGTGCTCGGGTCGGCTCAC |
| | | | GAGGGCTGCCTCCCGCCGTTCCCAGCGGACGTTTTC |
| | | | ATGATTCCTCAGTACGGGTATCTGACGCTTAATGAT |
| | | | GGAAGCCAGGCCGTGGGTCGTTCGTCCTTTTACTGC |
| | | | CTGGAATATTTCCCGTCGCAAATGCTAAGAACGGG |
| | | | TAACAACTTCCAGTTCAGCTACGAGTTTGAGAACG |
| | | | TACCTTTCCATAGCAGCTACGCTCACAGCCAAAGC |
| | | | CTGGACCGACTAATGAATCCACTCATCGACCAATA |
| | | | CTTGTACTATCTCTCAAAGACTATTAACGGTTCTGG |
| | | | ACAGAATCAACAAACGCTAAAATTCAGTGTGGCCG |
| | | | GACCCAGCAACATGGCTGTCCAGGGAAGAAACTAC |
| | | | ATACCTGGACCCAGCTACCGACAACAACGTGTCTC |
| | | | AACCACTGTGACTCAAAACAACAACAGCGAATTTG |
| | | | CTTGGCCTGGAGCTTCTTCTTGGGCTCTCAATGGAC |
| | | | GTAATAGCTTGATGAATCCTGGACCTGCTATGGCC |
| | | | AGCCACAAAGAAGGAGAGGACCGTTTCTTTCCTTT |
| | | | GTCTGGATCTTTAATTTTTGGCAAACAAGGAACTG |
| | | | GAAGAGACAACGTGGATGCGGACAAAGTCATGAT |
| | | | AACCAACGAAGAAGAAATTAAAACTACTAACCCG |
| | | | GTAGCAACGGAGTCCTATGGACAAGTGGCCACAAA |
| | | | CCACCAGAGTGCCCAAGCACAGGCGCAGACCGGCT |
| | | | GGGTTCAAAACCAAGGAATACTTCCGGGTATGGTT |
| | | | TGGCAGGACAGAGATGTGTACCTGCAAGGACCCAT |
| | | | TTGGGCCAAAATTCCTCACACGGACGGCAACTTTC |
| | | | ACCCTTCTCCGCTGATGGGAGGGTTTGGAATGAAG |
| | | | CACCCGCCTCCTCAGATCCTCATCAAAAACACACC |
| | | | TGTACCTGCGGATCCTCCAACGGCCTTCAACAAGG |
| | | | ACAAGCTGAACTCTTTCATCACCCAGTATTCTACTG |
| | | | GCCAAGTCAGCGTGGAGATCGAGTGGGAGCTGCAG |
| | | | AAGGAAAACAGCAAGCGCTGGAACCCGGAGATCC |
| | | | AGTACACTTCCAACTATTACAAGTCTAATAATGTTG |
| | | | AATTTGCTGTTAATACTGAAGGTGTATATAGTGAA |
| | | | CCCCGCCCCATTGGCACCAGATACCTGACTCGTAA |
| | | | TCTGTAATTGCTTGTTAATCAATAAACCGTTTAA |
| | | | TTCGTTTCAGTTGAACTTTGGTCTCTGCGGAGGTC |
| | | | CTGTATTAGAGGTCACGTGAGTGTTTTGCGACA |
| | | | TTTTGCGACACCATGTGGTCACGCTGGGTATTT |
| | | | AAGCCCGAGTGAGCACGCAGGGTCTCCATTTTG |
| | | | AAGCGGGAGGTTTGAAATCCCAATGGCGCGCCGA |
| | | | GCTTGGCTCGAGCATGGTCATAGCTGTTTCCTGTGT |
| | | | GAAATTGTTATCCGCTCACAATTCCACACAACATA |
| | | | CGAGCCGGAAGCATAAAGTGTAAAGCCTGGGGTGC |
| | | | CTAATGAGTGAGCTAACTCACATTAATTGCGTTGC |
| | | | GCTCACTGCCCGCTTTCCAGTCGGGAAACCTGTCGT |
| | | | GCCAGCTGCATTAATGAATCGGCCAACGCGCGGGG |
| | | | AGAGGCGGTTTGCGTATTGGGCGCTCTTCCGCTTCC |
| | | | TCGCTCACTGACTCGCTGCGCTCGGTCGTTCGGCTG |
| | | | CGGCGAGCGGTATCAGCTCACTCAAAGGCGGTAAT |
| | | | ACGGTTATCCACAGAATCAGGGGATAACGCAGGAA |
| | | | AGAACATGTGAGCAAAAGGCCAGCAAAAGGCCAG |
| | | | GAACCGTAAAAAGGCCGCGTTGCTGGCGTTTTTCC |
| | | | ATAGGCTCCGCCCCCCTGACGAGCATCACAAAAAT |
| | | | CGACGCTCAAGTCAGAGGTGGCGAAACCCGACAG |
| | | | GACTATAAAGATACCAGGCGTTTCCCCCTGGAAGC |
| | | | TCCCTCGTGCGCTCTCCTGTTCCGACCCTGCCGCTT |
| | | | ACCGGATACCTGTCCGCCTTTCTCCCTTCGGGAAGC |
| | | | GTGGCGCTTTCTCATAGCTCACGCTGTAGGTATCTC |
| | | | AGTTCGGTGTAGGTCGTTCGCTCCAAGCTGGGCTGT |
| | | | GTGCACGAACCCCCCGTTCAGCCCGACCGCTGCGC |
| | | | CTTATCCGGTAACTATCGTCTTGAGTCCAACCCGGT |
| | | | AAGACACGACTTATCGCCACTGGCAGCAGCCACTG |
| | | | GTAACAGGATTAGCAGAGCGAGGTATGTAGGCGGT |
| | | | GCTACAGAGTTCTTGAAGTGGTGGCCTAACTACGG |
| | | | CTACACTAGAAGAACAGTATTTGGTATCTGCGCTCT |
| | | | GCTGAAGCCAGTTACCTTCGGAAAAAGAGTTGGTA |
| | | | GCTCTTGATCCGGCAAACAAACCACCGCTGGTAGC |
| | | | GGTGGTTTTTTTGTTTGCAAGCAGCAGATTACGCGC |
| | | | AGAAAAAAAGGATCTCAAGAAGATCCTTTGATCTT |
| | | | TTCTACGGGGTCTGACGCTCAGTGGAACGAAAACT |
| | | | CACGTTAAGGGATTTTGGTCATGAGATTATCAAAA |
| | | | AGGATCTTCACCTAGATCCTTTTAAATTAAAAATGA |
| | | | AGTTTTAAATCAATCTAAAGTATATATGAGTAAAC |
| | | | TTGGTCTGACAGTTAGAAAAACTCATCGAGCATCA |
| | | | AATGAAACTGCAATTTATTCATATCAGGATTATCA |
| | | | ATACCATATTTTTGAAAAAGCCGTTTCTGTAATGAA |
| | | | GGAGAAAACTCACCGAGGCAGTTCCATAGGATGGC |
| | | | AAGATCCTGGTATCGGTCTGCGATTCCGACTCGTCC |
| | | | AACATCAATACAACCTATTAATTTCCCCTCGTCAAA |
| | | | AATAAGGTTATCAAGTGAGAAATCACCATGAGTGA |
| | | | CGACTGAATCCGGTGAGAATGGCAAAAGTTTATGC |
| | | | ATTTCTTTCCAGACTTGTTCAACAGGCCAGCCATTA |
| | | | CGCTCGTCATCAAAATCACTCGCATCAACCAAACC |
| | | | GTTATTCATTCGTGATTGCGCCTGAGCGAGACGAA |
| | | | ATACGCGATCGCTGTTAAAAGGACAATTACAAACA |
| | | | GGAATCGAATGCAACCGGCGCAGGAACACTGCCA |
| | | | GCGCATCAACAATATTTTCACCTGAATCAGGATATT |
| | | | CTTCTAATACCTGGAATGCTGTTTTCCCAGGGATCG |
| | | | CAGTGGTGAGTAACCATGCATCATCAGGAGTACGG |
| | | | ATAAAATGCTTGATGGTCGGAAGAGGCATAAATTC |
| | | | CGTCAGCCAGTTTAGTCTGACCATCTCATCTGTAAC |
| | | | ATCATTGGCAACGCTACCTTTGCCATGTTTCAGAAA |
| | | | CAACTCTGGCGCATCGGGCTTCCCATACAATCGAT |
| | | | AGATTGTCGCACCTGATTGCCCGACATTATCGCGA |
| | | | GCCCATTTATACCCATATAAATCAGCATCCATGTTG |
| | | | GAATTTAATCGCGGCCTAGAGCAAGACGTTTCCCG |
| | | | TTGAATATGGCTCATACTCTTCCTTTTTCAATATTAT |
| | | | TGAAGCATTTATCAGGGTTATTGTCTCATGAGCGG |
| | | | ATACATATTTGAATGTATTTAGAAAAATAAACAAA |
| | | | TAGGGGTTCCGCGCACATTTCCCCGAAAAGTGCCA |
| | | | CCTGACGTCTAAGAAACCATTATTATCATGACATTA |
| | | | ACCTATAAAAATAGGCGTATCACGAGGCCCTTTCG |
| | | | TC |
| 41 | pYZZ284 | p5v6-Rep2Ca9- | TCGCGCGTTTCGGTGATGACGGTGAAAACCTCTGA |
| | | P5 | CACATGCAGCTCCCGGAGACGGTCACAGCTTGTCT |
| | | | GTAAGCGGATGCCGGGAGCAGACAAGCCCGTCAG |
| | | | GGCGCGTCAGCGGGTGTTGGCGGGTGTCGGGGCTG |
| | | | GCTTAACTATGCGGCATCAGAGCAGATTGTACTGA |
| | | | GAGTGCACCATATGCGGTGTGAAATACCGCACAGA |
| | | | TGCGTAAGGAGAAAATACCGCATCAGGCGCCATTC |
| | | | GCCATTCAGGCTGCGCAACTGTTGGGAAGGGCGAT |
| | | | CGGTGCGGGCCTCTTCGCTATTACGCCAGCTGGCG |
| | | | AAAGGGGGATGTGCTGCAAGGCGATTAAGTTGGGT |
| | | | AACGCCAGGGTTTTCCCAGTCACGACGTTGTAAAA |
| | | | CGACGGCCAGTGAATTGACGCGTATTGggatGAGGT |
| | | | CCTGTATTAGAGGTCACGTGAGTGTCATGTGGT |
| | | | CACGCTGGGTATTTAAGCCCGAGTGAGCACGCA |
| | | | GGGTCTCCATTTTGAAGCGGGAGGTTTGAACGC |
| | | | GCAGCCGCCATGccggggttttacgagattgtgattaaggtccccagc |
| | | | gaccttgacgagcatctgcccggcatttctgacagctttgtgaactgggtggccga |
| | | | gaaggaatgggagttgccgccagattctgacatggatctgaatctgattgagcag |
| | | | gcacccctgaccgtggccgagaagctgcagcgcgactttctgacggaatggcgc |
| | | | cgtgtgagtaaggccccggaggctcttttctttgtgcaatttgagaagggagagag |
| | | | ctacttccacatgcacgtgctcgtggaaaccaccggggTGAAATCCATGG |
| | | | TTTTGGGACGTTTCCTGAGTCAGATTCGCGAAAAACTG |
| | | | ATTCAGAGAATTTACCGCGGGATCGAGCCGACTTTGC |
| | | | CAAACTGGTTCGCGGTCACAAAGACCAGAAATGGCGC |
| | | | CGGAGGCGGGAACAAGGTGGTGGATGAGTGCTACAT |
| | | | CCCCAATTACTTGCTCCCCAAAACCCAGCCTGAGCTCC |
| | | | AGTGGGCGTGGACTAATATGGAACAGTATTTAAGCGC |
| | | | CTGTTTGAATCTCACGGAGCGTAAACGGTTGGTGGCG |
| | | | CAGCATCTGACGCACGTGTCGCAGACGCAGGAGCAGA |
| | | | ACAAAGAGAATCAGAATCCCAATTCTGATGCGCCGGTG |
| | | | ATCAGATCAAAAACTTCAGCCAGGTACATGGAGCTGGT |
| | | | CGGGTGGCTCGTGGACAAGGGGATTACCTCGGAGAA |
| | | | GCAGTGGATCCAGGAGGACCAGGCCTCATACATCTCC |
| | | | TTCAATGCGGCCTCCAACTCGCGGTCCCAAATCAAGG |
| | | | CTGCCTTGGACAATGCGGGAAAGATTATGAGCCTGAC |
| | | | TAAAACCGCCCCCGACTACCTGGTGGGCCAGCAGCCC |
| | | | GTGGAGGACATTTCCAGCAATCGGATTTATAAAATTTT |
| | | | GGAACTAAACGGGTACGATCCCCAATATGCGGCTTCC |
| | | | GTCTTTCTGGGATGGGCCACGAAAAAGTTCGGCAAGA |
| | | | GGAACACCATCTGGCTGTTTGGGCCTGCAACTACCGG |
| | | | GAAGACCAACATCGCGGAGGCCATAGCCCACACTGTG |
| | | | CCCTTCTACGGGTGCGTAAACTGGACCAATGAGAACTT |
| | | | TCCCTTCAACGACTGTGTCGACAAGATGGTGATCTGGT |
| | | | GGGAGGAGGGGAAGATGACCGCCAAGGTCGTGGAGT |
| | | | CGGCCAAAGCCATTCTCGGAGGAAGCAAGGTGCGCGT |
| | | | GGACCAGAAATGCAAGTCCTCGGCCCAGATAGACCCG |
| | | | ACTCCCGTGATCGTCACCTCCAACACCAACATGTGCG |
| | | | CCGTGATTGACGGGAACTCAACGACCTTCGAACACCA |
| | | | GCAGCCGTTGCAAGACCGGATGTTCAAATTTGAACTCA |
| | | | CCCGCCGTCTGGATCATGACTTTGGGAAGGTCACCAA |
| | | | GCAGGAAGTCAAAGACTTTTTCCGGTGGGCAAAGGAT |
| | | | CACGTGGTTGAGGTGGAGCATGAATTCTACGTCAAAAA |
| | | | GGGTGGAGCCAAGAAAAGACCCGCCCCCAGTGACGC |
| | | | AGATATAAGTGAGCCCAAACGGGTGCGCGAGTCAGTT |
| | | | GCGCAGCCATCGACGTCAGACGCGGAAGCTTCGATCA |
| | | | ACTACGCAGACAGGTACCAAAACAAATGTTCTCGTCAC |
| | | | GTGGGCATGAATCTGATGCTGTTTCCCTGCAGACAATG |
| | | | CGAGAGAATGAATCAGAATTCAAATATCTGCTTCACTC |
| | | | ACGGACAGAAAGACTGTTTAGAGTGCTTTCCCGTGTCA |
| | | | GAATCTCAACCCGTTTCTGTCGTCAAAAAGGCGTATCA |
| | | | GAAACTGTGCTACATTCATCATATCATGGGAAAGGTGC |
| | | | CAGACGCTTGCACTGCCTGCGATCTGGTCAATGTGGA |
| | | | TTTGGATGACTGCATCTTTGAACAATAAATGATTTAAA |
| | | | TCAGGTATGGCTGCCGATGGTTATCTTCCAGATTGG |
| | | | CTCGAGGACAACCTTAGTGAAGGAATTCGCGAGTG |
| | | | GTGGGCTTTGAAACCTGGAGCCCCTCAACCCAAGG |
| | | | CAAATCAACAACATCAAGACAACGCTCGAGGTCTT |
| | | | GTGCTTCCGGGTTACAAATACCTTGGACCCGGCAA |
| | | | CGGACTCGACAAGGGGGAGCCGGTCAACGCAGCA |
| | | | GACGCGGCGGCCCTCGAGCACGACAAGGCCTACGA |
| | | | CCAGCAGCTCAAGGCCGGAGACAACCCGTACCTCA |
| | | | AGTACAACCACGCCGACGCCGAGTTCCAGGAGCGG |
| | | | CTCAAAGAAGATACGTCTTTTGGGGGCAACCTCGG |
| | | | GCGAGCAGTCTTCCAGGCCAAAAAGAGGCTTCTTG |
| | | | AACCTCTTGGTCTGGTTGAGGAAGCGGCTAAGACG |
| | | | GCTCCTGGAAAGAAGAGGCCTGTAGAGCAGTCTCC |
| | | | TCAGGAACCGGACTCCTCCGCGGGTATTGGCAAAT |
| | | | CGGGTGCACAGCCCGCTAAAAAGAGACTCAATTTC |
| | | | GGTCAGACTGGCGACACAGAGTCAGTCCCAGACCC |
| | | | TCAACCAATCGGAGAACCTCCCGCAGCCCCCTCAG |
| | | | GTGTGGGATCTCTTACAATGGCTTCAGGTGGTGGC |
| | | | GCACCAGTGGCAGACAATAACGAAGGTGCCGATG |
| | | | GAGTGGGTAGTTCCTCGGGAAATTGGCATTGCGAT |
| | | | TCCCAATGGCTGGGGGACAGAGTCATCACCACCAG |
| | | | CACCCGAACCTGGGCCCTGCCCACCTACAACAATC |
| | | | ACCTCTACAAGCAAATCTCCAACAGCACATCTGGA |
| | | | GGATCTTCAAATGACAACGCCTACTTCGGCTACAG |
| | | | CACCCCCTGGGGGTATTTTGACTTCAACAGATTCCA |
| | | | CTGCCACTTCTCACCACGTGACTGGCAGCGACTCAT |
| | | | CAACAACAACTGGGGATTCCGGCCTAAGCGACTCA |
| | | | ACTTCAAGCTCTTCAACATTCAGGTCAAAGAGGTT |
| | | | ACGGACAACAATGGAGTCAAGACCATCGCCAATAA |
| | | | CCTTACCAGCACGGTCCAGGTCTTCACGGACTCAG |
| | | | ACTATCAGCTCCCGTACGTGCTCGGGTCGGCTCAC |
| | | | GAGGGCTGCCTCCCGCCGTTCCCAGCGGACGTTTTC |
| | | | ATGATTCCTCAGTACGGGTATCTGACGCTTAATGAT |
| | | | GGAAGCCAGGCCGTGGGTCGTTCGTCCTTTTACTGC |
| | | | CTGGAATATTTCCCGTCGCAAATGCTAAGAACGGG |
| | | | TAACAACTTCCAGTTCAGCTACGAGTTTGAGAACG |
| | | | TACCTTTCCATAGCAGCTACGCTCACAGCCAAAGC |
| | | | CTGGACCGACTAATGAATCCACTCATCGACCAATA |
| | | | CTTGTACTATCTCTCAAAGACTATTAACGGTTCTGG |
| | | | ACAGAATCAACAAACGCTAAAATTCAGTGTGGCCG |
| | | | GACCCAGCAACATGGCTGTCCAGGGAAGAAACTAC |
| | | | ATACCTGGACCCAGCTACCGACAACAACGTGTCTC |
| | | | AACCACTGTGACTCAAAACAACAACAGCGAATTTG |
| | | | CTTGGCCTGGAGCTTCTTCTTGGGCTCTCAATGGAC |
| | | | GTAATAGCTTGATGAATCCTGGACCTGCTATGGCC |
| | | | AGCCACAAAGAAGGAGAGGACCGTTTCTTTCCTTT |
| | | | GTCTGGATCTTTAATTTTTGGCAAACAAGGAACTG |
| | | | GAAGAGACAACGTGGATGCGGACAAAGTCATGAT |
| | | | AACCAACGAAGAAGAAATTAAAACTACTAACCCG |
| | | | GTAGCAACGGAGTCCTATGGACAAGTGGCCACAAA |
| | | | CCACCAGAGTGCCCAAGCACAGGCGCAGACCGGCT |
| | | | GGGTTCAAAACCAAGGAATACTTCCGGGTATGGTT |
| | | | TGGCAGGACAGAGATGTGTACCTGCAAGGACCCAT |
| | | | TTGGGCCAAAATTCCTCACACGGACGGCAACTTTC |
| | | | ACCCTTCTCCGCTGATGGGAGGGTTTGGAATGAAG |
| | | | CACCCGCCTCCTCAGATCCTCATCAAAAACACACC |
| | | | TGTACCTGCGGATCCTCCAACGGCCTTCAACAAGG |
| | | | ACAAGCTGAACTCTTTCATCACCCAGTATTCTACTG |
| | | | GCCAAGTCAGCGTGGAGATCGAGTGGGAGCTGCAG |
| | | | AAGGAAAACAGCAAGCGCTGGAACCCGGAGATCC |
| | | | AGTACACTTCCAACTATTACAAGTCTAATAATGTTG |
| | | | AATTTGCTGTTAATACTGAAGGTGTATATAGTGAA |
| | | | CCCCGCCCCATTGGCACCAGATACCTGACTCGTAA |
| | | | TCTGTAATTGCTTGTTAATCAATAAACCGTTTAA |
| | | | TTCGTTTCAGTTGAACTTTGGTCTCTGCGGAGGTC |
| | | | CTGTATTAGAGGTCACGTGAGTGTTTTGCGACA |
| | | | TTTTGCGACACCATGTGGTCACGCTGGGTATTT |
| | | | AAGCCCGAGTGAGCACGCAGGGTCTCCATTTTG |
| | | | AAGCGGGAGGTTTGAAATCCCAATGGCGCGCCGA |
| | | | GCTTGGCTCGAGCATGGTCATAGCTGTTTCCTGTGT |
| | | | GAAATTGTTATCCGCTCACAATTCCACACAACATA |
| | | | CGAGCCGGAAGCATAAAGTGTAAAGCCTGGGGTGC |
| | | | CTAATGAGTGAGCTAACTCACATTAATTGCGTTGC |
| | | | GCTCACTGCCCGCTTTCCAGTCGGGAAACCTGTCGT |
| | | | GCCAGCTGCATTAATGAATCGGCCAACGCGCGGGG |
| | | | AGAGGCGGTTTGCGTATTGGGCGCTCTTCCGCTTCC |
| | | | TCGCTCACTGACTCGCTGCGCTCGGTCGTTCGGCTG |
| | | | CGGCGAGCGGTATCAGCTCACTCAAAGGCGGTAAT |
| | | | ACGGTTATCCACAGAATCAGGGGATAACGCAGGAA |
| | | | AGAACATGTGAGCAAAAGGCCAGCAAAAGGCCAG |
| | | | GAACCGTAAAAAGGCCGCGTTGCTGGCGTTTTTCC |
| | | | ATAGGCTCCGCCCCCCTGACGAGCATCACAAAAAT |
| | | | CGACGCTCAAGTCAGAGGTGGCGAAACCCGACAG |
| | | | GACTATAAAGATACCAGGCGTTTCCCCCTGGAAGC |
| | | | TCCCTCGTGCGCTCTCCTGTTCCGACCCTGCCGCTT |
| | | | ACCGGATACCTGTCCGCCTTTCTCCCTTCGGGAAGC |
| | | | GTGGCGCTTTCTCATAGCTCACGCTGTAGGTATCTC |
| | | | AGTTCGGTGTAGGTCGTTCGCTCCAAGCTGGGCTGT |
| | | | GTGCACGAACCCCCCGTTCAGCCCGACCGCTGCGC |
| | | | CTTATCCGGTAACTATCGTCTTGAGTCCAACCCGGT |
| | | | AAGACACGACTTATCGCCACTGGCAGCAGCCACTG |
| | | | GTAACAGGATTAGCAGAGCGAGGTATGTAGGCGGT |
| | | | GCTACAGAGTTCTTGAAGTGGTGGCCTAACTACGG |
| | | | CTACACTAGAAGAACAGTATTTGGTATCTGCGCTCT |
| | | | GCTGAAGCCAGTTACCTTCGGAAAAAGAGTTGGTA |
| | | | GCTCTTGATCCGGCAAACAAACCACCGCTGGTAGC |
| | | | GGTGGTTTTTTTGTTTGCAAGCAGCAGATTACGCGC |
| | | | AGAAAAAAAGGATCTCAAGAAGATCCTTTGATCTT |
| | | | TTCTACGGGGTCTGACGCTCAGTGGAACGAAAACT |
| | | | CACGTTAAGGGATTTTGGTCATGAGATTATCAAAA |
| | | | AGGATCTTCACCTAGATCCTTTTAAATTAAAAATGA |
| | | | AGTTTTAAATCAATCTAAAGTATATATGAGTAAAC |
| | | | TTGGTCTGACAGTTAGAAAAACTCATCGAGCATCA |
| | | | AATGAAACTGCAATTTATTCATATCAGGATTATCA |
| | | | ATACCATATTTTTGAAAAAGCCGTTTCTGTAATGAA |
| | | | GGAGAAAACTCACCGAGGCAGTTCCATAGGATGGC |
| | | | AAGATCCTGGTATCGGTCTGCGATTCCGACTCGTCC |
| | | | AACATCAATACAACCTATTAATTTCCCCTCGTCAAA |
| | | | AATAAGGTTATCAAGTGAGAAATCACCATGAGTGA |
| | | | CGACTGAATCCGGTGAGAATGGCAAAAGTTTATGC |
| | | | ATTTCTTTCCAGACTTGTTCAACAGGCCAGCCATTA |
| | | | CGCTCGTCATCAAAATCACTCGCATCAACCAAACC |
| | | | GTTATTCATTCGTGATTGCGCCTGAGCGAGACGAA |
| | | | ATACGCGATCGCTGTTAAAAGGACAATTACAAACA |
| | | | GGAATCGAATGCAACCGGCGCAGGAACACTGCCA |
| | | | GCGCATCAACAATATTTTCACCTGAATCAGGATATT |
| | | | CTTCTAATACCTGGAATGCTGTTTTCCCAGGGATCG |
| | | | CAGTGGTGAGTAACCATGCATCATCAGGAGTACGG |
| | | | ATAAAATGCTTGATGGTCGGAAGAGGCATAAATTC |
| | | | CGTCAGCCAGTTTAGTCTGACCATCTCATCTGTAAC |
| | | | ATCATTGGCAACGCTACCTTTGCCATGTTTCAGAAA |
| | | | CAACTCTGGCGCATCGGGCTTCCCATACAATCGAT |
| | | | AGATTGTCGCACCTGATTGCCCGACATTATCGCGA |
| | | | GCCCATTTATACCCATATAAATCAGCATCCATGTTG |
| | | | GAATTTAATCGCGGCCTAGAGCAAGACGTTTCCCG |
| | | | TTGAATATGGCTCATACTCTTCCTTTTTCAATATTAT |
| | | | TGAAGCATTTATCAGGGTTATTGTCTCATGAGCGG |
| | | | ATACATATTTGAATGTATTTAGAAAAATAAACAAA |
| | | | TAGGGGTTCCGCGCACATTTCCCCGAAAAGTGCCA |
| | | | CCTGACGTCTAAGAAACCATTATTATCATGACATTA |
| | | | ACCTATAAAAATAGGCGTATCACGAGGCCCTTTCG |
| | | | TC |
| 42 | pYZZ285 | p5v7-Rep2Ca9- | TCGCGCGTTTCGGTGATGACGGTGAAAACCTCTGA |
| | | P5 | CACATGCAGCTCCCGGAGACGGTCACAGCTTGTCT |
| | | | GTAAGCGGATGCCGGGAGCAGACAAGCCCGTCAG |
| | | | GGCGCGTCAGCGGGTGTTGGCGGGTGTCGGGGCTG |
| | | | GCTTAACTATGCGGCATCAGAGCAGATTGTACTGA |
| | | | GAGTGCACCATATGCGGTGTGAAATACCGCACAGA |
| | | | TGCGTAAGGAGAAAATACCGCATCAGGCGCCATTC |
| | | | GCCATTCAGGCTGCGCAACTGTTGGGAAGGGCGAT |
| | | | CGGTGCGGGCCTCTTCGCTATTACGCCAGCTGGCG |
| | | | AAAGGGGGATGTGCTGCAAGGCGATTAAGTTGGGT |
| | | | AACGCCAGGGTTTTCCCAGTCACGACGTTGTAAAA |
| | | | CGACGGCCAGTGAATTGACGCGTATTGggatGAGGT |
| | | | CCTGTATTAGACATGTGGTCACGCTGGGTATTT |
| | | | AAGCCCGAGTGAGCACGCAGGGTCTCCATTTTG |
| | | | AAGCGGGAGGTTTGAACGCGCAGCCGCCATGccg |
| | | | gggttttacgagattgtgattaaggtccccagcgaccttgacgagcatctgcccgg |
| | | | catttctgacagctttgtgaactgggtggccgagaaggaatgggagttgccgcca |
| | | | gattctgacatggatctgaatctgattgagcaggcacccctgaccgtggccgaga |
| | | | agctgcagcgcgactttctgacggaatggcgccgtgtgagtaaggccccggagg |
| | | | ctcttttctttgtgcaatttgagaagggagagagctacttccacatgcacgtgctcgt |
| | | | ggaaaccaccggggTGAAATCCATGGTTTTGGGACGTTTCCT |
| | | | GAGTCAGATTCGCGAAAAACTGATTCAGAGAATTTACC |
| | | | GCGGGATCGAGCCGACTTTGCCAAACTGGTTCGCGGT |
| | | | CACAAAGACCAGAAATGGCGCCGGAGGCGGGAACAA |
| | | | GGTGGTGGATGAGTGCTACATCCCCAATTACTTGCTCC |
| | | | CCAAAACCCAGCCTGAGCTCCAGTGGGCGTGGACTAA |
| | | | TATGGAACAGTATTTAAGCGCCTGTTTGAATCTCACGG |
| | | | AGCGTAAACGGTTGGTGGCGCAGCATCTGACGCACGT |
| | | | GTCGCAGACGCAGGAGCAGAACAAAGAGAATCAGAAT |
| | | | CCCAATTCTGATGCGCCGGTGATCAGATCAAAAACTTC |
| | | | AGCCAGGTACATGGAGCTGGTCGGGTGGCTCGTGGA |
| | | | CAAGGGGATTACCTCGGAGAAGCAGTGGATCCAGGAG |
| | | | GACCAGGCCTCATACATCTCCTTCAATGCGGCCTCCAA |
| | | | CTCGCGGTCCCAAATCAAGGCTGCCTTGGACAATGCG |
| | | | GGAAAGATTATGAGCCTGACTAAAACCGCCCCCGACT |
| | | | ACCTGGTGGGCCAGCAGCCCGTGGAGGACATTTCCAG |
| | | | CAATCGGATTTATAAAATTTTGGAACTAAACGGGTACG |
| | | | ATCCCCAATATGCGGCTTCCGTCTTTCTGGGATGGGC |
| | | | CACGAAAAAGTTCGGCAAGAGGAACACCATCTGGCTG |
| | | | TTTGGGCCTGCAACTACCGGGAAGACCAACATCGCGG |
| | | | AGGCCATAGCCCACACTGTGCCCTTCTACGGGTGCGT |
| | | | AAACTGGACCAATGAGAACTTTCCCTTCAACGACTGTG |
| | | | TCGACAAGATGGTGATCTGGTGGGAGGAGGGGAAGAT |
| | | | GACCGCCAAGGTCGTGGAGTCGGCCAAAGCCATTCTC |
| | | | GGAGGAAGCAAGGTGCGCGTGGACCAGAAATGCAAG |
| | | | TCCTCGGCCCAGATAGACCCGACTCCCGTGATCGTCA |
| | | | CCTCCAACACCAACATGTGCGCCGTGATTGACGGGAA |
| | | | CTCAACGACCTTCGAACACCAGCAGCCGTTGCAAGAC |
| | | | CGGATGTTCAAATTTGAACTCACCCGCCGTCTGGATCA |
| | | | TGACTTTGGGAAGGTCACCAAGCAGGAAGTCAAAGAC |
| | | | TTTTTCCGGTGGGCAAAGGATCACGTGGTTGAGGTGG |
| | | | AGCATGAATTCTACGTCAAAAAGGGTGGAGCCAAGAAA |
| | | | AGACCCGCCCCCAGTGACGCAGATATAAGTGAGCCCA |
| | | | AACGGGTGCGCGAGTCAGTTGCGCAGCCATCGACGTC |
| | | | AGACGCGGAAGCTTCGATCAACTACGCAGACAGGTAC |
| | | | CAAAACAAATGTTCTCGTCACGTGGGCATGAATCTGAT |
| | | | GCTGTTTCCCTGCAGACAATGCGAGAGAATGAATCAGA |
| | | | ATTCAAATATCTGCTTCACTCACGGACAGAAAGACTGT |
| | | | TTAGAGTGCTTTCCCGTGTCAGAATCTCAACCCGTTTC |
| | | | TGTCGTCAAAAAGGCGTATCAGAAACTGTGCTACATTC |
| | | | ATCATATCATGGGAAAGGTGCCAGACGCTTGCACTGC |
| | | | CTGCGATCTGGTCAATGTGGATTTGGATGACTGCATCT |
| | | | TTGAACAATAAATGATTTAAATCAGGTATGGCTGCC |
| | | | GATGGTTATCTTCCAGATTGGCTCGAGGACAACCTT |
| | | | AGTGAAGGAATTCGCGAGTGGTGGGCTTTGAAACC |
| | | | TGGAGCCCCTCAACCCAAGGCAAATCAACAACATC |
| | | | AAGACAACGCTCGAGGTCTTGTGCTTCCGGGTTAC |
| | | | AAATACCTTGGACCCGGCAACGGACTCGACAAGGG |
| | | | GGAGCCGGTCAACGCAGCAGACGCGGCGGCCCTCG |
| | | | AGCACGACAAGGCCTACGACCAGCAGCTCAAGGCC |
| | | | GGAGACAACCCGTACCTCAAGTACAACCACGCCGA |
| | | | CGCCGAGTTCCAGGAGCGGCTCAAAGAAGATACGT |
| | | | CTTTTGGGGGCAACCTCGGGCGAGCAGTCTTCCAG |
| | | | GCCAAAAAGAGGCTTCTTGAACCTCTTGGTCTGGTT |
| | | | GAGGAAGCGGCTAAGACGGCTCCTGGAAAGAAGA |
| | | | GGCCTGTAGAGCAGTCTCCTCAGGAACCGGACTCC |
| | | | TCCGCGGGTATTGGCAAATCGGGTGCACAGCCCGC |
| | | | TAAAAAGAGACTCAATTTCGGTCAGACTGGCGACA |
| | | | CAGAGTCAGTCCCAGACCCTCAACCAATCGGAGAA |
| | | | CCTCCCGCAGCCCCCTCAGGTGTGGGATCTCTTACA |
| | | | ATGGCTTCAGGTGGTGGCGCACCAGTGGCAGACAA |
| | | | TAACGAAGGTGCCGATGGAGTGGGTAGTTCCTCGG |
| | | | GAAATTGGCATTGCGATTCCCAATGGCTGGGGGAC |
| | | | AGAGTCATCACCACCAGCACCCGAACCTGGGCCCT |
| | | | GCCCACCTACAACAATCACCTCTACAAGCAAATCT |
| | | | CCAACAGCACATCTGGAGGATCTTCAAATGACAAC |
| | | | GCCTACTTCGGCTACAGCACCCCCTGGGGGTATTTT |
| | | | GACTTCAACAGATTCCACTGCCACTTCTCACCACGT |
| | | | GACTGGCAGCGACTCATCAACAACAACTGGGGATT |
| | | | CCGGCCTAAGCGACTCAACTTCAAGCTCTTCAACA |
| | | | TTCAGGTCAAAGAGGTTACGGACAACAATGGAGTC |
| | | | AAGACCATCGCCAATAACCTTACCAGCACGGTCCA |
| | | | GGTCTTCACGGACTCAGACTATCAGCTCCCGTACGT |
| | | | GCTCGGGTCGGCTCACGAGGGCTGCCTCCCGCCGT |
| | | | TCCCAGCGGACGTTTTCATGATTCCTCAGTACGGGT |
| | | | ATCTGACGCTTAATGATGGAAGCCAGGCCGTGGGT |
| | | | CGTTCGTCCTTTTACTGCCTGGAATATTTCCCGTCG |
| | | | CAAATGCTAAGAACGGGTAACAACTTCCAGTTCAG |
| | | | CTACGAGTTTGAGAACGTACCTTTCCATAGCAGCT |
| | | | ACGCTCACAGCCAAAGCCTGGACCGACTAATGAAT |
| | | | CCACTCATCGACCAATACTTGTACTATCTCTCAAAG |
| | | | ACTATTAACGGTTCTGGACAGAATCAACAAACGCT |
| | | | AAAATTCAGTGTGGCCGGACCCAGCAACATGGCTG |
| | | | TCCAGGGAAGAAACTACATACCTGGACCCAGCTAC |
| | | | CGACAACAACGTGTCTCAACCACTGTGACTCAAAA |
| | | | CAACAACAGCGAATTTGCTTGGCCTGGAGCTTCTTC |
| | | | TTGGGCTCTCAATGGACGTAATAGCTTGATGAATC |
| | | | CTGGACCTGCTATGGCCAGCCACAAAGAAGGAGAG |
| | | | GACCGTTTCTTTCCTTTGTCTGGATCTTTAATTTTTG |
| | | | GCAAACAAGGAACTGGAAGAGACAACGTGGATGC |
| | | | GGACAAAGTCATGATAACCAACGAAGAAGAAATT |
| | | | AAAACTACTAACCCGGTAGCAACGGAGTCCTATGG |
| | | | ACAAGTGGCCACAAACCACCAGAGTGCCCAAGCAC |
| | | | AGGCGCAGACCGGCTGGGTTCAAAACCAAGGAAT |
| | | | ACTTCCGGGTATGGTTTGGCAGGACAGAGATGTGT |
| | | | ACCTGCAAGGACCCATTTGGGCCAAAATTCCTCAC |
| | | | ACGGACGGCAACTTTCACCCTTCTCCGCTGATGGG |
| | | | AGGGTTTGGAATGAAGCACCCGCCTCCTCAGATCC |
| | | | TCATCAAAAACACACCTGTACCTGCGGATCCTCCA |
| | | | ACGGCCTTCAACAAGGACAAGCTGAACTCTTTCAT |
| | | | CACCCAGTATTCTACTGGCCAAGTCAGCGTGGAGA |
| | | | TCGAGTGGGAGCTGCAGAAGGAAAACAGCAAGCG |
| | | | CTGGAACCCGGAGATCCAGTACACTTCCAACTATT |
| | | | ACAAGTCTAATAATGTTGAATTTGCTGTTAATACTG |
| | | | AAGGTGTATATAGTGAACCCCGCCCCATTGGCACC |
| | | | AGATACCTGACTCGTAATCTGTAATTGCTTGTTAA |
| | | | TCAATAAACCGTTTAATTCGTTTCAGTTGAACTT |
| | | | TGGTCTCTGCGGAGGTCCTGTATTAGAGGTCACG |
| | | | TGAGTGTTTTGCGACATTTTGCGACACCATGTG |
| | | | GTCACGCTGGGTATTTAAGCCCGAGTGAGCACG |
| | | | CAGGGTCTCCATTTTGAAGCGGGAGGTTTGAAA |
| | | | TCCCAATGGCGCGCCGAGCTTGGCTCGAGCATGGT |
| | | | CATAGCTGTTTCCTGTGTGAAATTGTTATCCGCTCA |
| | | | CAATTCCACACAACATACGAGCCGGAAGCATAAAG |
| | | | TGTAAAGCCTGGGGTGCCTAATGAGTGAGCTAACT |
| | | | CACATTAATTGCGTTGCGCTCACTGCCCGCTTTCCA |
| | | | GTCGGGAAACCTGTCGTGCCAGCTGCATTAATGAA |
| | | | TCGGCCAACGCGCGGGGAGAGGCGGTTTGCGTATT |
| | | | GGGCGCTCTTCCGCTTCCTCGCTCACTGACTCGCTG |
| | | | CGCTCGGTCGTTCGGCTGCGGCGAGCGGTATCAGC |
| | | | TCACTCAAAGGCGGTAATACGGTTATCCACAGAAT |
| | | | CAGGGGATAACGCAGGAAAGAACATGTGAGCAAA |
| | | | AGGCCAGCAAAAGGCCAGGAACCGTAAAAAGGCC |
| | | | GCGTTGCTGGCGTTTTTCCATAGGCTCCGCCCCCCT |
| | | | GACGAGCATCACAAAAATCGACGCTCAAGTCAGAG |
| | | | GTGGCGAAACCCGACAGGACTATAAAGATACCAG |
| | | | GCGTTTCCCCCTGGAAGCTCCCTCGTGCGCTCTCCT |
| | | | GTTCCGACCCTGCCGCTTACCGGATACCTGTCCGCC |
| | | | TTTCTCCCTTCGGGAAGCGTGGCGCTTTCTCATAGC |
| | | | TCACGCTGTAGGTATCTCAGTTCGGTGTAGGTCGTT |
| | | | CGCTCCAAGCTGGGCTGTGTGCACGAACCCCCCGT |
| | | | TCAGCCCGACCGCTGCGCCTTATCCGGTAACTATCG |
| | | | TCTTGAGTCCAACCCGGTAAGACACGACTTATCGC |
| | | | CACTGGCAGCAGCCACTGGTAACAGGATTAGCAGA |
| | | | GCGAGGTATGTAGGCGGTGCTACAGAGTTCTTGAA |
| | | | GTGGTGGCCTAACTACGGCTACACTAGAAGAACAG |
| | | | TATTTGGTATCTGCGCTCTGCTGAAGCCAGTTACCT |
| | | | TCGGAAAAAGAGTTGGTAGCTCTTGATCCGGCAAA |
| | | | CAAACCACCGCTGGTAGCGGTGGTTTTTTTGTTTGC |
| | | | AAGCAGCAGATTACGCGCAGAAAAAAAGGATCTC |
| | | | AAGAAGATCCTTTGATCTTTTCTACGGGGTCTGACG |
| | | | CTCAGTGGAACGAAAACTCACGTTAAGGGATTTTG |
| | | | GTCATGAGATTATCAAAAAGGATCTTCACCTAGAT |
| | | | CCTTTTAAATTAAAAATGAAGTTTTAAATCAATCTA |
| | | | AAGTATATATGAGTAAACTTGGTCTGACAGTTAGA |
| | | | AAAACTCATCGAGCATCAAATGAAACTGCAATTTA |
| | | | TTCATATCAGGATTATCAATACCATATTTTTGAAAA |
| | | | AGCCGTTTCTGTAATGAAGGAGAAAACTCACCGAG |
| | | | GCAGTTCCATAGGATGGCAAGATCCTGGTATCGGT |
| | | | CTGCGATTCCGACTCGTCCAACATCAATACAACCT |
| | | | ATTAATTTCCCCTCGTCAAAAATAAGGTTATCAAGT |
| | | | GAGAAATCACCATGAGTGACGACTGAATCCGGTGA |
| | | | GAATGGCAAAAGTTTATGCATTTCTTTCCAGACTTG |
| | | | TTCAACAGGCCAGCCATTACGCTCGTCATCAAAAT |
| | | | CACTCGCATCAACCAAACCGTTATTCATTCGTGATT |
| | | | GCGCCTGAGCGAGACGAAATACGCGATCGCTGTTA |
| | | | AAAGGACAATTACAAACAGGAATCGAATGCAACC |
| | | | GGCGCAGGAACACTGCCAGCGCATCAACAATATTT |
| | | | TCACCTGAATCAGGATATTCTTCTAATACCTGGAAT |
| | | | GCTGTTTTCCCAGGGATCGCAGTGGTGAGTAACCA |
| | | | TGCATCATCAGGAGTACGGATAAAATGCTTGATGG |
| | | | TCGGAAGAGGCATAAATTCCGTCAGCCAGTTTAGT |
| | | | CTGACCATCTCATCTGTAACATCATTGGCAACGCTA |
| | | | CCTTTGCCATGTTTCAGAAACAACTCTGGCGCATCG |
| | | | GGCTTCCCATACAATCGATAGATTGTCGCACCTGAT |
| | | | TGCCCGACATTATCGCGAGCCCATTTATACCCATAT |
| | | | AAATCAGCATCCATGTTGGAATTTAATCGCGGCCT |
| | | | AGAGCAAGACGTTTCCCGTTGAATATGGCTCATAC |
| | | | TCTTCCTTTTTCAATATTATTGAAGCATTTATCAGG |
| | | | GTTATTGTCTCATGAGCGGATACATATTTGAATGTA |
| | | | TTTAGAAAAATAAACAAATAGGGGTTCCGCGCACA |
| | | | TTTCCCCGAAAAGTGCCACCTGACGTCTAAGAAAC |
| | | | CATTATTATCATGACATTAACCTATAAAAATAGGC |
| | | | GTATCACGAGGCCCTTTCGTC |
| 43 | pYZZ286 | p5v8-Rep2Ca9- | TCGCGCGTTTCGGTGATGACGGTGAAAACCTCTGA |
| | | P5 | CACATGCAGCTCCCGGAGACGGTCACAGCTTGTCT |
| | | | GTAAGCGGATGCCGGGAGCAGACAAGCCCGTCAG |
| | | | GGCGCGTCAGCGGGTGTTGGCGGGTGTCGGGGCTG |
| | | | GCTTAACTATGCGGCATCAGAGCAGATTGTACTGA |
| | | | GAGTGCACCATATGCGGTGTGAAATACCGCACAGA |
| | | | TGCGTAAGGAGAAAATACCGCATCAGGCGCCATTC |
| | | | GCCATTCAGGCTGCGCAACTGTTGGGAAGGGCGAT |
| | | | CGGTGCGGGCCTCTTCGCTATTACGCCAGCTGGCG |
| | | | AAAGGGGGATGTGCTGCAAGGCGATTAAGTTGGGT |
| | | | AACGCCAGGGTTTTCCCAGTCACGACGTTGTAAAA |
| | | | CGACGGCCAGTGAATTGACGCGTATTGggatGAGGT |
| | | | CCTGTATTAGATTTGCGACATTTTGCGACACCAT |
| | | | GTGGTCACGCTGGGGCCCGAGTGAGCACGCAG |
| | | | GGTCTCCATTTTGAAGCGGGAGGTTTGAACGCG |
| | | | CAGCCGCCATGccggggttttacgagattgtgattaaggtccccagcga |
| | | | ccttgacgagcatctgcccggcatttctgacagctttgtgaactgggtggccgaga |
| | | | aggaatgggagttgccgccagattctgacatggatctgaatctgattgagcaggc |
| | | | acccctgaccgtggccgagaagctgcagcgcgactttctgacggaatggcgccg |
| | | | tgtgagtaaggccccggaggctcttttctttgtgcaatttgagaagggagagagct |
| | | | acttccacatgcacgtgctcgtggaaaccaccggggTGAAATCCATGGT |
| | | | TTTGGGACGTTTCCTGAGTCAGATTCGCGAAAAACTGA |
| | | | TTCAGAGAATTTACCGCGGGATCGAGCCGACTTTGCC |
| | | | AAACTGGTTCGCGGTCACAAAGACCAGAAATGGCGCC |
| | | | GGAGGCGGGAACAAGGTGGTGGATGAGTGCTACATC |
| | | | CCCAATTACTTGCTCCCCAAAACCCAGCCTGAGCTCCA |
| | | | GTGGGCGTGGACTAATATGGAACAGTATTTAAGCGCCT |
| | | | GTTTGAATCTCACGGAGCGTAAACGGTTGGTGGCGCA |
| | | | GCATCTGACGCACGTGTCGCAGACGCAGGAGCAGAAC |
| | | | AAAGAGAATCAGAATCCCAATTCTGATGCGCCGGTGAT |
| | | | CAGATCAAAAACTTCAGCCAGGTACATGGAGCTGGTC |
| | | | GGGTGGCTCGTGGACAAGGGGATTACCTCGGAGAAG |
| | | | CAGTGGATCCAGGAGGACCAGGCCTCATACATCTCCT |
| | | | TCAATGCGGCCTCCAACTCGCGGTCCCAAATCAAGGC |
| | | | TGCCTTGGACAATGCGGGAAAGATTATGAGCCTGACTA |
| | | | AAACCGCCCCCGACTACCTGGTGGGCCAGCAGCCCG |
| | | | TGGAGGACATTTCCAGCAATCGGATTTATAAAATTTTG |
| | | | GAACTAAACGGGTACGATCCCCAATATGCGGCTTCCG |
| | | | TCTTTCTGGGATGGGCCACGAAAAAGTTCGGCAAGAG |
| | | | GAACACCATCTGGCTGTTTGGGCCTGCAACTACCGGG |
| | | | AAGACCAACATCGCGGAGGCCATAGCCCACACTGTGC |
| | | | CCTTCTACGGGTGCGTAAACTGGACCAATGAGAACTTT |
| | | | CCCTTCAACGACTGTGTCGACAAGATGGTGATCTGGT |
| | | | GGGAGGAGGGGAAGATGACCGCCAAGGTCGTGGAGT |
| | | | CGGCCAAAGCCATTCTCGGAGGAAGCAAGGTGCGCGT |
| | | | GGACCAGAAATGCAAGTCCTCGGCCCAGATAGACCCG |
| | | | ACTCCCGTGATCGTCACCTCCAACACCAACATGTGCG |
| | | | CCGTGATTGACGGGAACTCAACGACCTTCGAACACCA |
| | | | GCAGCCGTTGCAAGACCGGATGTTCAAATTTGAACTCA |
| | | | CCCGCCGTCTGGATCATGACTTTGGGAAGGTCACCAA |
| | | | GCAGGAAGTCAAAGACTTTTTCCGGTGGGCAAAGGAT |
| | | | CACGTGGTTGAGGTGGAGCATGAATTCTACGTCAAAAA |
| | | | GGGTGGAGCCAAGAAAAGACCCGCCCCCAGTGACGC |
| | | | AGATATAAGTGAGCCCAAACGGGTGCGCGAGTCAGTT |
| | | | GCGCAGCCATCGACGTCAGACGCGGAAGCTTCGATCA |
| | | | ACTACGCAGACAGGTACCAAAACAAATGTTCTCGTCAC |
| | | | GTGGGCATGAATCTGATGCTGTTTCCCTGCAGACAATG |
| | | | CGAGAGAATGAATCAGAATTCAAATATCTGCTTCACTC |
| | | | ACGGACAGAAAGACTGTTTAGAGTGCTTTCCCGTGTCA |
| | | | GAATCTCAACCCGTTTCTGTCGTCAAAAAGGCGTATCA |
| | | | GAAACTGTGCTACATTCATCATATCATGGGAAAGGTGC |
| | | | CAGACGCTTGCACTGCCTGCGATCTGGTCAATGTGGA |
| | | | TTTGGATGACTGCATCTTTGAACAATAAATGATTTAAA |
| | | | TCAGGTATGGCTGCCGATGGTTATCTTCCAGATTGG |
| | | | CTCGAGGACAACCTTAGTGAAGGAATTCGCGAGTG |
| | | | GTGGGCTTTGAAACCTGGAGCCCCTCAACCCAAGG |
| | | | CAAATCAACAACATCAAGACAACGCTCGAGGTCTT |
| | | | GTGCTTCCGGGTTACAAATACCTTGGACCCGGCAA |
| | | | CGGACTCGACAAGGGGGAGCCGGTCAACGCAGCA |
| | | | GACGCGGCGGCCCTCGAGCACGACAAGGCCTACGA |
| | | | CCAGCAGCTCAAGGCCGGAGACAACCCGTACCTCA |
| | | | AGTACAACCACGCCGACGCCGAGTTCCAGGAGCGG |
| | | | CTCAAAGAAGATACGTCTTTTGGGGGCAACCTCGG |
| | | | GCGAGCAGTCTTCCAGGCCAAAAAGAGGCTTCTTG |
| | | | AACCTCTTGGTCTGGTTGAGGAAGCGGCTAAGACG |
| | | | GCTCCTGGAAAGAAGAGGCCTGTAGAGCAGTCTCC |
| | | | TCAGGAACCGGACTCCTCCGCGGGTATTGGCAAAT |
| | | | CGGGTGCACAGCCCGCTAAAAAGAGACTCAATTTC |
| | | | GGTCAGACTGGCGACACAGAGTCAGTCCCAGACCC |
| | | | TCAACCAATCGGAGAACCTCCCGCAGCCCCCTCAG |
| | | | GTGTGGGATCTCTTACAATGGCTTCAGGTGGTGGC |
| | | | GCACCAGTGGCAGACAATAACGAAGGTGCCGATG |
| | | | GAGTGGGTAGTTCCTCGGGAAATTGGCATTGCGAT |
| | | | TCCCAATGGCTGGGGGACAGAGTCATCACCACCAG |
| | | | CACCCGAACCTGGGCCCTGCCCACCTACAACAATC |
| | | | ACCTCTACAAGCAAATCTCCAACAGCACATCTGGA |
| | | | GGATCTTCAAATGACAACGCCTACTTCGGCTACAG |
| | | | CACCCCCTGGGGGTATTTTGACTTCAACAGATTCCA |
| | | | CTGCCACTTCTCACCACGTGACTGGCAGCGACTCAT |
| | | | CAACAACAACTGGGGATTCCGGCCTAAGCGACTCA |
| | | | ACTTCAAGCTCTTCAACATTCAGGTCAAAGAGGTT |
| | | | ACGGACAACAATGGAGTCAAGACCATCGCCAATAA |
| | | | CCTTACCAGCACGGTCCAGGTCTTCACGGACTCAG |
| | | | ACTATCAGCTCCCGTACGTGCTCGGGTCGGCTCAC |
| | | | GAGGGCTGCCTCCCGCCGTTCCCAGCGGACGTTTTC |
| | | | ATGATTCCTCAGTACGGGTATCTGACGCTTAATGAT |
| | | | GGAAGCCAGGCCGTGGGTCGTTCGTCCTTTTACTGC |
| | | | CTGGAATATTTCCCGTCGCAAATGCTAAGAACGGG |
| | | | TAACAACTTCCAGTTCAGCTACGAGTTTGAGAACG |
| | | | TACCTTTCCATAGCAGCTACGCTCACAGCCAAAGC |
| | | | CTGGACCGACTAATGAATCCACTCATCGACCAATA |
| | | | CTTGTACTATCTCTCAAAGACTATTAACGGTTCTGG |
| | | | ACAGAATCAACAAACGCTAAAATTCAGTGTGGCCG |
| | | | GACCCAGCAACATGGCTGTCCAGGGAAGAAACTAC |
| | | | ATACCTGGACCCAGCTACCGACAACAACGTGTCTC |
| | | | AACCACTGTGACTCAAAACAACAACAGCGAATTTG |
| | | | CTTGGCCTGGAGCTTCTTCTTGGGCTCTCAATGGAC |
| | | | GTAATAGCTTGATGAATCCTGGACCTGCTATGGCC |
| | | | AGCCACAAAGAAGGAGAGGACCGTTTCTTTCCTTT |
| | | | GTCTGGATCTTTAATTTTTGGCAAACAAGGAACTG |
| | | | GAAGAGACAACGTGGATGCGGACAAAGTCATGAT |
| | | | AACCAACGAAGAAGAAATTAAAACTACTAACCCG |
| | | | GTAGCAACGGAGTCCTATGGACAAGTGGCCACAAA |
| | | | CCACCAGAGTGCCCAAGCACAGGCGCAGACCGGCT |
| | | | GGGTTCAAAACCAAGGAATACTTCCGGGTATGGTT |
| | | | TGGCAGGACAGAGATGTGTACCTGCAAGGACCCAT |
| | | | TTGGGCCAAAATTCCTCACACGGACGGCAACTTTC |
| | | | ACCCTTCTCCGCTGATGGGAGGGTTTGGAATGAAG |
| | | | CACCCGCCTCCTCAGATCCTCATCAAAAACACACC |
| | | | TGTACCTGCGGATCCTCCAACGGCCTTCAACAAGG |
| | | | ACAAGCTGAACTCTTTCATCACCCAGTATTCTACTG |
| | | | GCCAAGTCAGCGTGGAGATCGAGTGGGAGCTGCAG |
| | | | AAGGAAAACAGCAAGCGCTGGAACCCGGAGATCC |
| | | | AGTACACTTCCAACTATTACAAGTCTAATAATGTTG |
| | | | AATTTGCTGTTAATACTGAAGGTGTATATAGTGAA |
| | | | CCCCGCCCCATTGGCACCAGATACCTGACTCGTAA |
| | | | TCTGTAATTGCTTGTTAATCAATAAACCGTTTAA |
| | | | TTCGTTTCAGTTGAACTTTGGTCTCTGCGGAGGTC |
| | | | CTGTATTAGAGGTCACGTGAGTGTTTTGCGACA |
| | | | TTTTGCGACACCATGTGGTCACGCTGGGTATTT |
| | | | AAGCCCGAGTGAGCACGCAGGGTCTCCATTTTG |
| | | | AAGCGGGAGGTTTGAAATCCCAATGGCGCGCCGA |
| | | | GCTTGGCTCGAGCATGGTCATAGCTGTTTCCTGTGT |
| | | | GAAATTGTTATCCGCTCACAATTCCACACAACATA |
| | | | CGAGCCGGAAGCATAAAGTGTAAAGCCTGGGGTGC |
| | | | CTAATGAGTGAGCTAACTCACATTAATTGCGTTGC |
| | | | GCTCACTGCCCGCTTTCCAGTCGGGAAACCTGTCGT |
| | | | GCCAGCTGCATTAATGAATCGGCCAACGCGCGGGG |
| | | | AGAGGCGGTTTGCGTATTGGGCGCTCTTCCGCTTCC |
| | | | TCGCTCACTGACTCGCTGCGCTCGGTCGTTCGGCTG |
| | | | CGGCGAGCGGTATCAGCTCACTCAAAGGCGGTAAT |
| | | | ACGGTTATCCACAGAATCAGGGGATAACGCAGGAA |
| | | | AGAACATGTGAGCAAAAGGCCAGCAAAAGGCCAG |
| | | | GAACCGTAAAAAGGCCGCGTTGCTGGCGTTTTTCC |
| | | | ATAGGCTCCGCCCCCCTGACGAGCATCACAAAAAT |
| | | | CGACGCTCAAGTCAGAGGTGGCGAAACCCGACAG |
| | | | GACTATAAAGATACCAGGCGTTTCCCCCTGGAAGC |
| | | | TCCCTCGTGCGCTCTCCTGTTCCGACCCTGCCGCTT |
| | | | ACCGGATACCTGTCCGCCTTTCTCCCTTCGGGAAGC |
| | | | GTGGCGCTTTCTCATAGCTCACGCTGTAGGTATCTC |
| | | | AGTTCGGTGTAGGTCGTTCGCTCCAAGCTGGGCTGT |
| | | | GTGCACGAACCCCCCGTTCAGCCCGACCGCTGCGC |
| | | | CTTATCCGGTAACTATCGTCTTGAGTCCAACCCGGT |
| | | | AAGACACGACTTATCGCCACTGGCAGCAGCCACTG |
| | | | GTAACAGGATTAGCAGAGCGAGGTATGTAGGCGGT |
| | | | GCTACAGAGTTCTTGAAGTGGTGGCCTAACTACGG |
| | | | CTACACTAGAAGAACAGTATTTGGTATCTGCGCTCT |
| | | | GCTGAAGCCAGTTACCTTCGGAAAAAGAGTTGGTA |
| | | | GCTCTTGATCCGGCAAACAAACCACCGCTGGTAGC |
| | | | GGTGGTTTTTTTGTTTGCAAGCAGCAGATTACGCGC |
| | | | AGAAAAAAAGGATCTCAAGAAGATCCTTTGATCTT |
| | | | TTCTACGGGGTCTGACGCTCAGTGGAACGAAAACT |
| | | | CACGTTAAGGGATTTTGGTCATGAGATTATCAAAA |
| | | | AGGATCTTCACCTAGATCCTTTTAAATTAAAAATGA |
| | | | AGTTTTAAATCAATCTAAAGTATATATGAGTAAAC |
| | | | TTGGTCTGACAGTTAGAAAAACTCATCGAGCATCA |
| | | | AATGAAACTGCAATTTATTCATATCAGGATTATCA |
| | | | ATACCATATTTTTGAAAAAGCCGTTTCTGTAATGAA |
| | | | GGAGAAAACTCACCGAGGCAGTTCCATAGGATGGC |
| | | | AAGATCCTGGTATCGGTCTGCGATTCCGACTCGTCC |
| | | | AACATCAATACAACCTATTAATTTCCCCTCGTCAAA |
| | | | AATAAGGTTATCAAGTGAGAAATCACCATGAGTGA |
| | | | CGACTGAATCCGGTGAGAATGGCAAAAGTTTATGC |
| | | | ATTTCTTTCCAGACTTGTTCAACAGGCCAGCCATTA |
| | | | CGCTCGTCATCAAAATCACTCGCATCAACCAAACC |
| | | | GTTATTCATTCGTGATTGCGCCTGAGCGAGACGAA |
| | | | ATACGCGATCGCTGTTAAAAGGACAATTACAAACA |
| | | | GGAATCGAATGCAACCGGCGCAGGAACACTGCCA |
| | | | GCGCATCAACAATATTTTCACCTGAATCAGGATATT |
| | | | CTTCTAATACCTGGAATGCTGTTTTCCCAGGGATCG |
| | | | CAGTGGTGAGTAACCATGCATCATCAGGAGTACGG |
| | | | ATAAAATGCTTGATGGTCGGAAGAGGCATAAATTC |
| | | | CGTCAGCCAGTTTAGTCTGACCATCTCATCTGTAAC |
| | | | ATCATTGGCAACGCTACCTTTGCCATGTTTCAGAAA |
| | | | CAACTCTGGCGCATCGGGCTTCCCATACAATCGAT |
| | | | AGATTGTCGCACCTGATTGCCCGACATTATCGCGA |
| | | | GCCCATTTATACCCATATAAATCAGCATCCATGTTG |
| | | | GAATTTAATCGCGGCCTAGAGCAAGACGTTTCCCG |
| | | | TTGAATATGGCTCATACTCTTCCTTTTTCAATATTAT |
| | | | TGAAGCATTTATCAGGGTTATTGTCTCATGAGCGG |
| | | | ATACATATTTGAATGTATTTAGAAAAATAAACAAA |
| | | | TAGGGGTTCCGCGCACATTTCCCCGAAAAGTGCCA |
| | | | CCTGACGTCTAAGAAACCATTATTATCATGACATTA |
| | | | ACCTATAAAAATAGGCGTATCACGAGGCCCTTTCG |
| | | | TC |
| 44 | pYZZ287 | p5v9-Rep2Ca9- | TCGCGCGTTTCGGTGATGACGGTGAAAACCTCTGA |
| | | P5 | CACATGCAGCTCCCGGAGACGGTCACAGCTTGTCT |
| | | | GTAAGCGGATGCCGGGAGCAGACAAGCCCGTCAG |
| | | | GGCGCGTCAGCGGGTGTTGGCGGGTGTCGGGGCTG |
| | | | GCTTAACTATGCGGCATCAGAGCAGATTGTACTGA |
| | | | GAGTGCACCATATGCGGTGTGAAATACCGCACAGA |
| | | | TGCGTAAGGAGAAAATACCGCATCAGGCGCCATTC |
| | | | GCCATTCAGGCTGCGCAACTGTTGGGAAGGGCGAT |
| | | | CGGTGCGGGCCTCTTCGCTATTACGCCAGCTGGCG |
| | | | AAAGGGGGATGTGCTGCAAGGCGATTAAGTTGGGT |
| | | | AACGCCAGGGTTTTCCCAGTCACGACGTTGTAAAA |
| | | | CGACGGCCAGTGAATTGACGCGTATTGggatGAGGT |
| | | | CCTGTATTAGAGGTCACGTGAGTGTCATGTGGT |
| | | | CACGCTGGGGCCCGAGTGAGCACGCAGGGTCTC |
| | | | CATTTTGAAGCGGGAGGTTTGAACGCGCAGCCG |
| | | | CCATGccggggttttacgagattgtgattaaggtccccagcgaccttgacgag |
| | | | catctgcccggcatttctgacagctttgtgaactgggtggccgagaaggaatggg |
| | | | agttgccgccagattctgacatggatctgaatctgattgagcaggcacccctgacc |
| | | | gtggccgagaagctgcagcgcgactttctgacggaatggcgccgtgtgagtaag |
| | | | gccccggaggctcttttctttgtgcaatttgagaagggagagagctacttccacatg |
| | | | cacgtgctcgtggaaaccaccggggTGAAATCCATGGTTTTGGGA |
| | | | CGTTTCCTGAGTCAGATTCGCGAAAAACTGATTCAGAG |
| | | | AATTTACCGCGGGATCGAGCCGACTTTGCCAAACTGG |
| | | | TTCGCGGTCACAAAGACCAGAAATGGCGCCGGAGGC |
| | | | GGGAACAAGGTGGTGGATGAGTGCTACATCCCCAATT |
| | | | ACTTGCTCCCCAAAACCCAGCCTGAGCTCCAGTGGGC |
| | | | GTGGACTAATATGGAACAGTATTTAAGCGCCTGTTTGA |
| | | | ATCTCACGGAGCGTAAACGGTTGGTGGCGCAGCATCT |
| | | | GACGCACGTGTCGCAGACGCAGGAGCAGAACAAAGA |
| | | | GAATCAGAATCCCAATTCTGATGCGCCGGTGATCAGAT |
| | | | CAAAAACTTCAGCCAGGTACATGGAGCTGGTCGGGTG |
| | | | GCTCGTGGACAAGGGGATTACCTCGGAGAAGCAGTGG |
| | | | ATCCAGGAGGACCAGGCCTCATACATCTCCTTCAATGC |
| | | | GGCCTCCAACTCGCGGTCCCAAATCAAGGCTGCCTTG |
| | | | GACAATGCGGGAAAGATTATGAGCCTGACTAAAACCG |
| | | | CCCCCGACTACCTGGTGGGCCAGCAGCCCGTGGAGG |
| | | | ACATTTCCAGCAATCGGATTTATAAAATTTTGGAACTAA |
| | | | ACGGGTACGATCCCCAATATGCGGCTTCCGTCTTTCTG |
| | | | GGATGGGCCACGAAAAAGTTCGGCAAGAGGAACACCA |
| | | | TCTGGCTGTTTGGGCCTGCAACTACCGGGAAGACCAA |
| | | | CATCGCGGAGGCCATAGCCCACACTGTGCCCTTCTAC |
| | | | GGGTGCGTAAACTGGACCAATGAGAACTTTCCCTTCAA |
| | | | CGACTGTGTCGACAAGATGGTGATCTGGTGGGAGGAG |
| | | | GGGAAGATGACCGCCAAGGTCGTGGAGTCGGCCAAA |
| | | | GCCATTCTCGGAGGAAGCAAGGTGCGCGTGGACCAG |
| | | | AAATGCAAGTCCTCGGCCCAGATAGACCCGACTCCCG |
| | | | TGATCGTCACCTCCAACACCAACATGTGCGCCGTGATT |
| | | | GACGGGAACTCAACGACCTTCGAACACCAGCAGCCGT |
| | | | TGCAAGACCGGATGTTCAAATTTGAACTCACCCGCCGT |
| | | | CTGGATCATGACTTTGGGAAGGTCACCAAGCAGGAAG |
| | | | TCAAAGACTTTTTCCGGTGGGCAAAGGATCACGTGGTT |
| | | | GAGGTGGAGCATGAATTCTACGTCAAAAAGGGTGGAG |
| | | | CCAAGAAAAGACCCGCCCCCAGTGACGCAGATATAAG |
| | | | TGAGCCCAAACGGGTGCGCGAGTCAGTTGCGCAGCC |
| | | | ATCGACGTCAGACGCGGAAGCTTCGATCAACTACGCA |
| | | | GACAGGTACCAAAACAAATGTTCTCGTCACGTGGGCAT |
| | | | GAATCTGATGCTGTTTCCCTGCAGACAATGCGAGAGAA |
| | | | TGAATCAGAATTCAAATATCTGCTTCACTCACGGACAG |
| | | | AAAGACTGTTTAGAGTGCTTTCCCGTGTCAGAATCTCA |
| | | | ACCCGTTTCTGTCGTCAAAAAGGCGTATCAGAAACTGT |
| | | | GCTACATTCATCATATCATGGGAAAGGTGCCAGACGCT |
| | | | TGCACTGCCTGCGATCTGGTCAATGTGGATTTGGATGA |
| | | | CTGCATCTTTGAACAATAAATGATTTAAATCAGGTAT |
| | | | GGCTGCCGATGGTTATCTTCCAGATTGGCTCGAGG |
| | | | ACAACCTTAGTGAAGGAATTCGCGAGTGGTGGGCT |
| | | | TTGAAACCTGGAGCCCCTCAACCCAAGGCAAATCA |
| | | | ACAACATCAAGACAACGCTCGAGGTCTTGTGCTTC |
| | | | CGGGTTACAAATACCTTGGACCCGGCAACGGACTC |
| | | | GACAAGGGGGAGCCGGTCAACGCAGCAGACGCGG |
| | | | CGGCCCTCGAGCACGACAAGGCCTACGACCAGCAG |
| | | | CTCAAGGCCGGAGACAACCCGTACCTCAAGTACAA |
| | | | CCACGCCGACGCCGAGTTCCAGGAGCGGCTCAAAG |
| | | | AAGATACGTCTTTTGGGGGCAACCTCGGGCGAGCA |
| | | | GTCTTCCAGGCCAAAAAGAGGCTTCTTGAACCTCTT |
| | | | GGTCTGGTTGAGGAAGCGGCTAAGACGGCTCCTGG |
| | | | AAAGAAGAGGCCTGTAGAGCAGTCTCCTCAGGAAC |
| | | | CGGACTCCTCCGCGGGTATTGGCAAATCGGGTGCA |
| | | | CAGCCCGCTAAAAAGAGACTCAATTTCGGTCAGAC |
| | | | TGGCGACACAGAGTCAGTCCCAGACCCTCAACCAA |
| | | | TCGGAGAACCTCCCGCAGCCCCCTCAGGTGTGGGA |
| | | | TCTCTTACAATGGCTTCAGGTGGTGGCGCACCAGT |
| | | | GGCAGACAATAACGAAGGTGCCGATGGAGTGGGT |
| | | | AGTTCCTCGGGAAATTGGCATTGCGATTCCCAATG |
| | | | GCTGGGGGACAGAGTCATCACCACCAGCACCCGAA |
| | | | CCTGGGCCCTGCCCACCTACAACAATCACCTCTAC |
| | | | AAGCAAATCTCCAACAGCACATCTGGAGGATCTTC |
| | | | AAATGACAACGCCTACTTCGGCTACAGCACCCCCT |
| | | | GGGGGTATTTTGACTTCAACAGATTCCACTGCCACT |
| | | | TCTCACCACGTGACTGGCAGCGACTCATCAACAAC |
| | | | AACTGGGGATTCCGGCCTAAGCGACTCAACTTCAA |
| | | | GCTCTTCAACATTCAGGTCAAAGAGGTTACGGACA |
| | | | ACAATGGAGTCAAGACCATCGCCAATAACCTTACC |
| | | | AGCACGGTCCAGGTCTTCACGGACTCAGACTATCA |
| | | | GCTCCCGTACGTGCTCGGGTCGGCTCACGAGGGCT |
| | | | GCCTCCCGCCGTTCCCAGCGGACGTTTTCATGATTC |
| | | | CTCAGTACGGGTATCTGACGCTTAATGATGGAAGC |
| | | | CAGGCCGTGGGTCGTTCGTCCTTTTACTGCCTGGAA |
| | | | TATTTCCCGTCGCAAATGCTAAGAACGGGTAACAA |
| | | | CTTCCAGTTCAGCTACGAGTTTGAGAACGTACCTTT |
| | | | CCATAGCAGCTACGCTCACAGCCAAAGCCTGGACC |
| | | | GACTAATGAATCCACTCATCGACCAATACTTGTACT |
| | | | ATCTCTCAAAGACTATTAACGGTTCTGGACAGAAT |
| | | | CAACAAACGCTAAAATTCAGTGTGGCCGGACCCAG |
| | | | CAACATGGCTGTCCAGGGAAGAAACTACATACCTG |
| | | | GACCCAGCTACCGACAACAACGTGTCTCAACCACT |
| | | | GTGACTCAAAACAACAACAGCGAATTTGCTTGGCC |
| | | | TGGAGCTTCTTCTTGGGCTCTCAATGGACGTAATAG |
| | | | CTTGATGAATCCTGGACCTGCTATGGCCAGCCACA |
| | | | AAGAAGGAGAGGACCGTTTCTTTCCTTTGTCTGGAT |
| | | | CTTTAATTTTTGGCAAACAAGGAACTGGAAGAGAC |
| | | | AACGTGGATGCGGACAAAGTCATGATAACCAACGA |
| | | | AGAAGAAATTAAAACTACTAACCCGGTAGCAACGG |
| | | | AGTCCTATGGACAAGTGGCCACAAACCACCAGAGT |
| | | | GCCCAAGCACAGGCGCAGACCGGCTGGGTTCAAAA |
| | | | CCAAGGAATACTTCCGGGTATGGTTTGGCAGGACA |
| | | | GAGATGTGTACCTGCAAGGACCCATTTGGGCCAAA |
| | | | ATTCCTCACACGGACGGCAACTTTCACCCTTCTCCG |
| | | | CTGATGGGAGGGTTTGGAATGAAGCACCCGCCTCC |
| | | | TCAGATCCTCATCAAAAACACACCTGTACCTGCGG |
| | | | ATCCTCCAACGGCCTTCAACAAGGACAAGCTGAAC |
| | | | TCTTTCATCACCCAGTATTCTACTGGCCAAGTCAGC |
| | | | GTGGAGATCGAGTGGGAGCTGCAGAAGGAAAACA |
| | | | GCAAGCGCTGGAACCCGGAGATCCAGTACACTTCC |
| | | | AACTATTACAAGTCTAATAATGTTGAATTTGCTGTT |
| | | | AATACTGAAGGTGTATATAGTGAACCCCGCCCCAT |
| | | | TGGCACCAGATACCTGACTCGTAATCTGTAATTGC |
| | | | TTGTTAATCAATAAACCGTTTAATTCGTTTCAGT |
| | | | TGAACTTTGGTCTCTGCGGAGGTCCTGTATTAGA |
| | | | GGTCACGTGAGTGTTTTGCGACATTTTGCGACA |
| | | | CCATGTGGTCACGCTGGGTATTTAAGCCCGAGT |
| | | | GAGCACGCAGGGTCTCCATTTTGAAGCGGGAGG |
| | | | TTTGAAATCCCAATGGCGCGCCGAGCTTGGCTCGA |
| | | | GCATGGTCATAGCTGTTTCCTGTGTGAAATTGTTAT |
| | | | CCGCTCACAATTCCACACAACATACGAGCCGGAAG |
| | | | CATAAAGTGTAAAGCCTGGGGTGCCTAATGAGTGA |
| | | | GCTAACTCACATTAATTGCGTTGCGCTCACTGCCCG |
| | | | CTTTCCAGTCGGGAAACCTGTCGTGCCAGCTGCATT |
| | | | AATGAATCGGCCAACGCGCGGGGAGAGGCGGTTTG |
| | | | CGTATTGGGCGCTCTTCCGCTTCCTCGCTCACTGAC |
| | | | TCGCTGCGCTCGGTCGTTCGGCTGCGGCGAGCGGT |
| | | | ATCAGCTCACTCAAAGGCGGTAATACGGTTATCCA |
| | | | CAGAATCAGGGGATAACGCAGGAAAGAACATGTG |
| | | | AGCAAAAGGCCAGCAAAAGGCCAGGAACCGTAAA |
| | | | AAGGCCGCGTTGCTGGCGTTTTTCCATAGGCTCCGC |
| | | | CCCCCTGACGAGCATCACAAAAATCGACGCTCAAG |
| | | | TCAGAGGTGGCGAAACCCGACAGGACTATAAAGAT |
| | | | ACCAGGCGTTTCCCCCTGGAAGCTCCCTCGTGCGCT |
| | | | CTCCTGTTCCGACCCTGCCGCTTACCGGATACCTGT |
| | | | CCGCCTTTCTCCCTTCGGGAAGCGTGGCGCTTTCTC |
| | | | ATAGCTCACGCTGTAGGTATCTCAGTTCGGTGTAG |
| | | | GTCGTTCGCTCCAAGCTGGGCTGTGTGCACGAACC |
| | | | CCCCGTTCAGCCCGACCGCTGCGCCTTATCCGGTAA |
| | | | CTATCGTCTTGAGTCCAACCCGGTAAGACACGACT |
| | | | TATCGCCACTGGCAGCAGCCACTGGTAACAGGATT |
| | | | AGCAGAGCGAGGTATGTAGGCGGTGCTACAGAGTT |
| | | | CTTGAAGTGGTGGCCTAACTACGGCTACACTAGAA |
| | | | GAACAGTATTTGGTATCTGCGCTCTGCTGAAGCCA |
| | | | GTTACCTTCGGAAAAAGAGTTGGTAGCTCTTGATC |
| | | | CGGCAAACAAACCACCGCTGGTAGCGGTGGTTTTT |
| | | | TTGTTTGCAAGCAGCAGATTACGCGCAGAAAAAAA |
| | | | GGATCTCAAGAAGATCCTTTGATCTTTTCTACGGGG |
| | | | TCTGACGCTCAGTGGAACGAAAACTCACGTTAAGG |
| | | | GATTTTGGTCATGAGATTATCAAAAAGGATCTTCA |
| | | | CCTAGATCCTTTTAAATTAAAAATGAAGTTTTAAAT |
| | | | CAATCTAAAGTATATATGAGTAAACTTGGTCTGAC |
| | | | AGTTAGAAAAACTCATCGAGCATCAAATGAAACTG |
| | | | CAATTTATTCATATCAGGATTATCAATACCATATTT |
| | | | TTGAAAAAGCCGTTTCTGTAATGAAGGAGAAAACT |
| | | | CACCGAGGCAGTTCCATAGGATGGCAAGATCCTGG |
| | | | TATCGGTCTGCGATTCCGACTCGTCCAACATCAATA |
| | | | CAACCTATTAATTTCCCCTCGTCAAAAATAAGGTTA |
| | | | TCAAGTGAGAAATCACCATGAGTGACGACTGAATC |
| | | | CGGTGAGAATGGCAAAAGTTTATGCATTTCTTTCCA |
| | | | GACTTGTTCAACAGGCCAGCCATTACGCTCGTCATC |
| | | | AAAATCACTCGCATCAACCAAACCGTTATTCATTC |
| | | | GTGATTGCGCCTGAGCGAGACGAAATACGCGATCG |
| | | | CTGTTAAAAGGACAATTACAAACAGGAATCGAATG |
| | | | CAACCGGCGCAGGAACACTGCCAGCGCATCAACAA |
| | | | TATTTTCACCTGAATCAGGATATTCTTCTAATACCT |
| | | | GGAATGCTGTTTTCCCAGGGATCGCAGTGGTGAGT |
| | | | AACCATGCATCATCAGGAGTACGGATAAAATGCTT |
| | | | GATGGTCGGAAGAGGCATAAATTCCGTCAGCCAGT |
| | | | TTAGTCTGACCATCTCATCTGTAACATCATTGGCAA |
| | | | CGCTACCTTTGCCATGTTTCAGAAACAACTCTGGCG |
| | | | CATCGGGCTTCCCATACAATCGATAGATTGTCGCA |
| | | | CCTGATTGCCCGACATTATCGCGAGCCCATTTATAC |
| | | | CCATATAAATCAGCATCCATGTTGGAATTTAATCGC |
| | | | GGCCTAGAGCAAGACGTTTCCCGTTGAATATGGCT |
| | | | CATACTCTTCCTTTTTCAATATTATTGAAGCATTTAT |
| | | | CAGGGTTATTGTCTCATGAGCGGATACATATTTGA |
| | | | ATGTATTTAGAAAAATAAACAAATAGGGGTTCCGC |
| | | | GCACATTTCCCCGAAAAGTGCCACCTGACGTCTAA |
| | | | GAAACCATTATTATCATGACATTAACCTATAAAAA |
| | | | TAGGCGTATCACGAGGCCCTTTCGTC |
| 45 | pYZZ288 | p5v10-Rep2Ca9- | TCGCGCGTTTCGGTGATGACGGTGAAAACCTCTGA |
| | | P5 | CACATGCAGCTCCCGGAGACGGTCACAGCTTGTCT |
| | | | GTAAGCGGATGCCGGGAGCAGACAAGCCCGTCAG |
| | | | GGCGCGTCAGCGGGTGTTGGCGGGTGTCGGGGCTG |
| | | | GCTTAACTATGCGGCATCAGAGCAGATTGTACTGA |
| | | | GAGTGCACCATATGCGGTGTGAAATACCGCACAGA |
| | | | TGCGTAAGGAGAAAATACCGCATCAGGCGCCATTC |
| | | | GCCATTCAGGCTGCGCAACTGTTGGGAAGGGCGAT |
| | | | CGGTGCGGGCCTCTTCGCTATTACGCCAGCTGGCG |
| | | | AAAGGGGGATGTGCTGCAAGGCGATTAAGTTGGGT |
| | | | AACGCCAGGGTTTTCCCAGTCACGACGTTGTAAAA |
| | | | CGACGGCCAGTGAATTGACGCGTATTGggatGAGGT |
| | | | CCTGTATTAGACATGTGGTCACGCTGGGGCCCG |
| | | | AGTGAGCACGCAGGGTCTCCATTTTGAAGCGGG |
| | | | AGGTTTGAACGCGCAGCCGCCATGccggggttttacgaga |
| | | | ttgtgattaaggtccccagcgaccttgacgagcatctgcccggcatttctgacagct |
| | | | ttgtgaactgggtggccgagaaggaatgggagttgccgccagattctgacatgga |
| | | | tctgaatctgattgagcaggcacccctgaccgtggccgagaagctgcagcgcga |
| | | | ctttctgacggaatggcgccgtgtgagtaaggccccggaggctcttttctttgtgca |
| | | | atttgagaagggagagagctacttccacatgcacgtgctcgtggaaaccaccgg |
| | | | ggTGAAATCCATGGTTTTGGGACGTTTCCTGAGTCAGAT |
| | | | TCGCGAAAAACTGATTCAGAGAATTTACCGCGGGATCG |
| | | | AGCCGACTTTGCCAAACTGGTTCGCGGTCACAAAGAC |
| | | | CAGAAATGGCGCCGGAGGCGGGAACAAGGTGGTGGA |
| | | | TGAGTGCTACATCCCCAATTACTTGCTCCCCAAAACCC |
| | | | AGCCTGAGCTCCAGTGGGCGTGGACTAATATGGAACA |
| | | | GTATTTAAGCGCCTGTTTGAATCTCACGGAGCGTAAAC |
| | | | GGTTGGTGGCGCAGCATCTGACGCACGTGTCGCAGAC |
| | | | GCAGGAGCAGAACAAAGAGAATCAGAATCCCAATTCT |
| | | | GATGCGCCGGTGATCAGATCAAAAACTTCAGCCAGGT |
| | | | ACATGGAGCTGGTCGGGTGGCTCGTGGACAAGGGGA |
| | | | TTACCTCGGAGAAGCAGTGGATCCAGGAGGACCAGGC |
| | | | CTCATACATCTCCTTCAATGCGGCCTCCAACTCGCGGT |
| | | | CCCAAATCAAGGCTGCCTTGGACAATGCGGGAAAGAT |
| | | | TATGAGCCTGACTAAAACCGCCCCCGACTACCTGGTG |
| | | | GGCCAGCAGCCCGTGGAGGACATTTCCAGCAATCGGA |
| | | | TTTATAAAATTTTGGAACTAAACGGGTACGATCCCCAAT |
| | | | ATGCGGCTTCCGTCTTTCTGGGATGGGCCACGAAAAA |
| | | | GTTCGGCAAGAGGAACACCATCTGGCTGTTTGGGCCT |
| | | | GCAACTACCGGGAAGACCAACATCGCGGAGGCCATAG |
| | | | CCCACACTGTGCCCTTCTACGGGTGCGTAAACTGGAC |
| | | | CAATGAGAACTTTCCCTTCAACGACTGTGTCGACAAGA |
| | | | TGGTGATCTGGTGGGAGGAGGGGAAGATGACCGCCA |
| | | | AGGTCGTGGAGTCGGCCAAAGCCATTCTCGGAGGAAG |
| | | | CAAGGTGCGCGTGGACCAGAAATGCAAGTCCTCGGCC |
| | | | CAGATAGACCCGACTCCCGTGATCGTCACCTCCAACA |
| | | | CCAACATGTGCGCCGTGATTGACGGGAACTCAACGAC |
| | | | CTTCGAACACCAGCAGCCGTTGCAAGACCGGATGTTC |
| | | | AAATTTGAACTCACCCGCCGTCTGGATCATGACTTTGG |
| | | | GAAGGTCACCAAGCAGGAAGTCAAAGACTTTTTCCGGT |
| | | | GGGCAAAGGATCACGTGGTTGAGGTGGAGCATGAATT |
| | | | CTACGTCAAAAAGGGTGGAGCCAAGAAAAGACCCGCC |
| | | | CCCAGTGACGCAGATATAAGTGAGCCCAAACGGGTGC |
| | | | GCGAGTCAGTTGCGCAGCCATCGACGTCAGACGCGG |
| | | | AAGCTTCGATCAACTACGCAGACAGGTACCAAAACAAA |
| | | | TGTTCTCGTCACGTGGGCATGAATCTGATGCTGTTTCC |
| | | | CTGCAGACAATGCGAGAGAATGAATCAGAATTCAAATA |
| | | | TCTGCTTCACTCACGGACAGAAAGACTGTTTAGAGTGC |
| | | | TTTCCCGTGTCAGAATCTCAACCCGTTTCTGTCGTCAA |
| | | | AAAGGCGTATCAGAAACTGTGCTACATTCATCATATCAT |
| | | | GGGAAAGGTGCCAGACGCTTGCACTGCCTGCGATCTG |
| | | | GTCAATGTGGATTTGGATGACTGCATCTTTGAACAATA |
| | | | AATGATTTAAATCAGGTATGGCTGCCGATGGTTATC |
| | | | TTCCAGATTGGCTCGAGGACAACCTTAGTGAAGGA |
| | | | ATTCGCGAGTGGTGGGCTTTGAAACCTGGAGCCCC |
| | | | TCAACCCAAGGCAAATCAACAACATCAAGACAACG |
| | | | CTCGAGGTCTTGTGCTTCCGGGTTACAAATACCTTG |
| | | | GACCCGGCAACGGACTCGACAAGGGGGAGCCGGT |
| | | | CAACGCAGCAGACGCGGCGGCCCTCGAGCACGAC |
| | | | AAGGCCTACGACCAGCAGCTCAAGGCCGGAGACA |
| | | | ACCCGTACCTCAAGTACAACCACGCCGACGCCGAG |
| | | | TTCCAGGAGCGGCTCAAAGAAGATACGTCTTTTGG |
| | | | GGGCAACCTCGGGCGAGCAGTCTTCCAGGCCAAAA |
| | | | AGAGGCTTCTTGAACCTCTTGGTCTGGTTGAGGAA |
| | | | GCGGCTAAGACGGCTCCTGGAAAGAAGAGGCCTGT |
| | | | AGAGCAGTCTCCTCAGGAACCGGACTCCTCCGCGG |
| | | | GTATTGGCAAATCGGGTGCACAGCCCGCTAAAAAG |
| | | | AGACTCAATTTCGGTCAGACTGGCGACACAGAGTC |
| | | | AGTCCCAGACCCTCAACCAATCGGAGAACCTCCCG |
| | | | CAGCCCCCTCAGGTGTGGGATCTCTTACAATGGCTT |
| | | | CAGGTGGTGGCGCACCAGTGGCAGACAATAACGA |
| | | | AGGTGCCGATGGAGTGGGTAGTTCCTCGGGAAATT |
| | | | GGCATTGCGATTCCCAATGGCTGGGGGACAGAGTC |
| | | | ATCACCACCAGCACCCGAACCTGGGCCCTGCCCAC |
| | | | CTACAACAATCACCTCTACAAGCAAATCTCCAACA |
| | | | GCACATCTGGAGGATCTTCAAATGACAACGCCTAC |
| | | | TTCGGCTACAGCACCCCCTGGGGGTATTTTGACTTC |
| | | | AACAGATTCCACTGCCACTTCTCACCACGTGACTG |
| | | | GCAGCGACTCATCAACAACAACTGGGGATTCCGGC |
| | | | CTAAGCGACTCAACTTCAAGCTCTTCAACATTCAG |
| | | | GTCAAAGAGGTTACGGACAACAATGGAGTCAAGA |
| | | | CCATCGCCAATAACCTTACCAGCACGGTCCAGGTC |
| | | | TTCACGGACTCAGACTATCAGCTCCCGTACGTGCTC |
| | | | GGGTCGGCTCACGAGGGCTGCCTCCCGCCGTTCCC |
| | | | AGCGGACGTTTTCATGATTCCTCAGTACGGGTATCT |
| | | | GACGCTTAATGATGGAAGCCAGGCCGTGGGTCGTT |
| | | | CGTCCTTTTACTGCCTGGAATATTTCCCGTCGCAAA |
| | | | TGCTAAGAACGGGTAACAACTTCCAGTTCAGCTAC |
| | | | GAGTTTGAGAACGTACCTTTCCATAGCAGCTACGC |
| | | | TCACAGCCAAAGCCTGGACCGACTAATGAATCCAC |
| | | | TCATCGACCAATACTTGTACTATCTCTCAAAGACTA |
| | | | TTAACGGTTCTGGACAGAATCAACAAACGCTAAAA |
| | | | TTCAGTGTGGCCGGACCCAGCAACATGGCTGTCCA |
| | | | GGGAAGAAACTACATACCTGGACCCAGCTACCGAC |
| | | | AACAACGTGTCTCAACCACTGTGACTCAAAACAAC |
| | | | AACAGCGAATTTGCTTGGCCTGGAGCTTCTTCTTGG |
| | | | GCTCTCAATGGACGTAATAGCTTGATGAATCCTGG |
| | | | ACCTGCTATGGCCAGCCACAAAGAAGGAGAGGAC |
| | | | CGTTTCTTTCCTTTGTCTGGATCTTTAATTTTTGGCA |
| | | | AACAAGGAACTGGAAGAGACAACGTGGATGCGGA |
| | | | CAAAGTCATGATAACCAACGAAGAAGAAATTAAA |
| | | | ACTACTAACCCGGTAGCAACGGAGTCCTATGGACA |
| | | | AGTGGCCACAAACCACCAGAGTGCCCAAGCACAG |
| | | | GCGCAGACCGGCTGGGTTCAAAACCAAGGAATACT |
| | | | TCCGGGTATGGTTTGGCAGGACAGAGATGTGTACC |
| | | | TGCAAGGACCCATTTGGGCCAAAATTCCTCACACG |
| | | | GACGGCAACTTTCACCCTTCTCCGCTGATGGGAGG |
| | | | GTTTGGAATGAAGCACCCGCCTCCTCAGATCCTCAT |
| | | | CAAAAACACACCTGTACCTGCGGATCCTCCAACGG |
| | | | CCTTCAACAAGGACAAGCTGAACTCTTTCATCACC |
| | | | CAGTATTCTACTGGCCAAGTCAGCGTGGAGATCGA |
| | | | GTGGGAGCTGCAGAAGGAAAACAGCAAGCGCTGG |
| | | | AACCCGGAGATCCAGTACACTTCCAACTATTACAA |
| | | | GTCTAATAATGTTGAATTTGCTGTTAATACTGAAGG |
| | | | TGTATATAGTGAACCCCGCCCCATTGGCACCAGAT |
| | | | ACCTGACTCGTAATCTGTAATTGCTTGTTAATCAA |
| | | | TAAACCGTTTAATTCGTTTCAGTTGAACTTTGGTC |
| | | | TCTGCGGAGGTCCTGTATTAGAGGTCACGTGAGT |
| | | | GTTTTGCGACATTTTGCGACACCATGTGGTCAC |
| | | | GCTGGGTATTTAAGCCCGAGTGAGCACGCAGGG |
| | | | TCTCCATTTTGAAGCGGGAGGTTTGAAATCCCAA |
| | | | TGGCGCGCCGAGCTTGGCTCGAGCATGGTCATAGC |
| | | | TGTTTCCTGTGTGAAATTGTTATCCGCTCACAATTC |
| | | | CACACAACATACGAGCCGGAAGCATAAAGTGTAA |
| | | | AGCCTGGGGTGCCTAATGAGTGAGCTAACTCACAT |
| | | | TAATTGCGTTGCGCTCACTGCCCGCTTTCCAGTCGG |
| | | | GAAACCTGTCGTGCCAGCTGCATTAATGAATCGGC |
| | | | CAACGCGCGGGGAGAGGCGGTTTGCGTATTGGGCG |
| | | | CTCTTCCGCTTCCTCGCTCACTGACTCGCTGCGCTC |
| | | | GGTCGTTCGGCTGCGGCGAGCGGTATCAGCTCACT |
| | | | CAAAGGCGGTAATACGGTTATCCACAGAATCAGGG |
| | | | GATAACGCAGGAAAGAACATGTGAGCAAAAGGCC |
| | | | AGCAAAAGGCCAGGAACCGTAAAAAGGCCGCGTT |
| | | | GCTGGCGTTTTTCCATAGGCTCCGCCCCCCTGACGA |
| | | | GCATCACAAAAATCGACGCTCAAGTCAGAGGTGGC |
| | | | GAAACCCGACAGGACTATAAAGATACCAGGCGTTT |
| | | | CCCCCTGGAAGCTCCCTCGTGCGCTCTCCTGTTCCG |
| | | | ACCCTGCCGCTTACCGGATACCTGTCCGCCTTTCTC |
| | | | CCTTCGGGAAGCGTGGCGCTTTCTCATAGCTCACGC |
| | | | TGTAGGTATCTCAGTTCGGTGTAGGTCGTTCGCTCC |
| | | | AAGCTGGGCTGTGTGCACGAACCCCCCGTTCAGCC |
| | | | CGACCGCTGCGCCTTATCCGGTAACTATCGTCTTGA |
| | | | GTCCAACCCGGTAAGACACGACTTATCGCCACTGG |
| | | | CAGCAGCCACTGGTAACAGGATTAGCAGAGCGAG |
| | | | GTATGTAGGCGGTGCTACAGAGTTCTTGAAGTGGT |
| | | | GGCCTAACTACGGCTACACTAGAAGAACAGTATTT |
| | | | GGTATCTGCGCTCTGCTGAAGCCAGTTACCTTCGGA |
| | | | AAAAGAGTTGGTAGCTCTTGATCCGGCAAACAAAC |
| | | | CACCGCTGGTAGCGGTGGTTTTTTTGTTTGCAAGCA |
| | | | GCAGATTACGCGCAGAAAAAAAGGATCTCAAGAA |
| | | | GATCCTTTGATCTTTTCTACGGGGTCTGACGCTCAG |
| | | | TGGAACGAAAACTCACGTTAAGGGATTTTGGTCAT |
| | | | GAGATTATCAAAAAGGATCTTCACCTAGATCCTTTT |
| | | | AAATTAAAAATGAAGTTTTAAATCAATCTAAAGTA |
| | | | TATATGAGTAAACTTGGTCTGACAGTTAGAAAAAC |
| | | | TCATCGAGCATCAAATGAAACTGCAATTTATTCAT |
| | | | ATCAGGATTATCAATACCATATTTTTGAAAAAGCC |
| | | | GTTTCTGTAATGAAGGAGAAAACTCACCGAGGCAG |
| | | | TTCCATAGGATGGCAAGATCCTGGTATCGGTCTGC |
| | | | GATTCCGACTCGTCCAACATCAATACAACCTATTA |
| | | | ATTTCCCCTCGTCAAAAATAAGGTTATCAAGTGAG |
| | | | AAATCACCATGAGTGACGACTGAATCCGGTGAGAA |
| | | | TGGCAAAAGTTTATGCATTTCTTTCCAGACTTGTTC |
| | | | AACAGGCCAGCCATTACGCTCGTCATCAAAATCAC |
| | | | TCGCATCAACCAAACCGTTATTCATTCGTGATTGCG |
| | | | CCTGAGCGAGACGAAATACGCGATCGCTGTTAAAA |
| | | | GGACAATTACAAACAGGAATCGAATGCAACCGGC |
| | | | GCAGGAACACTGCCAGCGCATCAACAATATTTTCA |
| | | | CCTGAATCAGGATATTCTTCTAATACCTGGAATGCT |
| | | | GTTTTCCCAGGGATCGCAGTGGTGAGTAACCATGC |
| | | | ATCATCAGGAGTACGGATAAAATGCTTGATGGTCG |
| | | | GAAGAGGCATAAATTCCGTCAGCCAGTTTAGTCTG |
| | | | ACCATCTCATCTGTAACATCATTGGCAACGCTACCT |
| | | | TTGCCATGTTTCAGAAACAACTCTGGCGCATCGGG |
| | | | CTTCCCATACAATCGATAGATTGTCGCACCTGATTG |
| | | | CCCGACATTATCGCGAGCCCATTTATACCCATATAA |
| | | | ATCAGCATCCATGTTGGAATTTAATCGCGGCCTAG |
| | | | AGCAAGACGTTTCCCGTTGAATATGGCTCATACTCT |
| | | | TCCTTTTTCAATATTATTGAAGCATTTATCAGGGTT |
| | | | ATTGTCTCATGAGCGGATACATATTTGAATGTATTT |
| | | | AGAAAAATAAACAAATAGGGGTTCCGCGCACATTT |
| | | | CCCCGAAAAGTGCCACCTGACGTCTAAGAAACCAT |
| | | | TATTATCATGACATTAACCTATAAAAATAGGCGTA |
| | | | TCACGAGGCCCTTTCGTC |
| 46 | pYZZ289 | p5v11-Rep2Ca9- | TCGCGCGTTTCGGTGATGACGGTGAAAACCTCTGA |
| | | P5 | CACATGCAGCTCCCGGAGACGGTCACAGCTTGTCT |
| | | | GTAAGCGGATGCCGGGAGCAGACAAGCCCGTCAG |
| | | | GGCGCGTCAGCGGGTGTTGGCGGGTGTCGGGGCTG |
| | | | GCTTAACTATGCGGCATCAGAGCAGATTGTACTGA |
| | | | GAGTGCACCATATGCGGTGTGAAATACCGCACAGA |
| | | | TGCGTAAGGAGAAAATACCGCATCAGGCGCCATTC |
| | | | GCCATTCAGGCTGCGCAACTGTTGGGAAGGGCGAT |
| | | | CGGTGCGGGCCTCTTCGCTATTACGCCAGCTGGCG |
| | | | AAAGGGGGATGTGCTGCAAGGCGATTAAGTTGGGT |
| | | | AACGCCAGGGTTTTCCCAGTCACGACGTTGTAAAA |
| | | | CGACGGCCAGTGAATTGACGCGTATTGggatGAGGT |
| | | | CCTGTATTAGAGCCCGAGTGAGCACGCAGGGTC |
| | | | TCCATTTTGAAGCGGGAGGTTTGAACGCGCAGC |
| | | | CGCCATGccggggttttacgagattgtgattaaggtccccagcgaccttgac |
| | | | gagcatctgcccggcatttctgacagctttgtgaactgggtggccgagaaggaat |
| | | | gggagttgccgccagattctgacatggatctgaatctgattgagcaggcacccctg |
| | | | accgtggccgagaagctgcagcgcgactttctgacggaatggcgccgtgtgagt |
| | | | aaggccccggaggctcttttctttgtgcaatttgagaagggagagagctacttcca |
| | | | catgcacgtgctcgtggaaaccaccggggTGAAATCCATGGTTTTG |
| | | | GGACGTTTCCTGAGTCAGATTCGCGAAAAACTGATTCA |
| | | | GAGAATTTACCGCGGGATCGAGCCGACTTTGCCAAAC |
| | | | TGGTTCGCGGTCACAAAGACCAGAAATGGCGCCGGAG |
| | | | GCGGGAACAAGGTGGTGGATGAGTGCTACATCCCCAA |
| | | | TTACTTGCTCCCCAAAACCCAGCCTGAGCTCCAGTGG |
| | | | GCGTGGACTAATATGGAACAGTATTTAAGCGCCTGTTT |
| | | | GAATCTCACGGAGCGTAAACGGTTGGTGGCGCAGCAT |
| | | | CTGACGCACGTGTCGCAGACGCAGGAGCAGAACAAAG |
| | | | AGAATCAGAATCCCAATTCTGATGCGCCGGTGATCAGA |
| | | | TCAAAAACTTCAGCCAGGTACATGGAGCTGGTCGGGT |
| | | | GGCTCGTGGACAAGGGGATTACCTCGGAGAAGCAGTG |
| | | | GATCCAGGAGGACCAGGCCTCATACATCTCCTTCAATG |
| | | | CGGCCTCCAACTCGCGGTCCCAAATCAAGGCTGCCTT |
| | | | GGACAATGCGGGAAAGATTATGAGCCTGACTAAAACC |
| | | | GCCCCCGACTACCTGGTGGGCCAGCAGCCCGTGGAG |
| | | | GACATTTCCAGCAATCGGATTTATAAAATTTTGGAACTA |
| | | | AACGGGTACGATCCCCAATATGCGGCTTCCGTCTTTCT |
| | | | GGGATGGGCCACGAAAAAGTTCGGCAAGAGGAACACC |
| | | | ATCTGGCTGTTTGGGCCTGCAACTACCGGGAAGACCA |
| | | | ACATCGCGGAGGCCATAGCCCACACTGTGCCCTTCTA |
| | | | CGGGTGCGTAAACTGGACCAATGAGAACTTTCCCTTCA |
| | | | ACGACTGTGTCGACAAGATGGTGATCTGGTGGGAGGA |
| | | | GGGGAAGATGACCGCCAAGGTCGTGGAGTCGGCCAA |
| | | | AGCCATTCTCGGAGGAAGCAAGGTGCGCGTGGACCA |
| | | | GAAATGCAAGTCCTCGGCCCAGATAGACCCGACTCCC |
| | | | GTGATCGTCACCTCCAACACCAACATGTGCGCCGTGA |
| | | | TTGACGGGAACTCAACGACCTTCGAACACCAGCAGCC |
| | | | GTTGCAAGACCGGATGTTCAAATTTGAACTCACCCGCC |
| | | | GTCTGGATCATGACTTTGGGAAGGTCACCAAGCAGGA |
| | | | AGTCAAAGACTTTTTCCGGTGGGCAAAGGATCACGTG |
| | | | GTTGAGGTGGAGCATGAATTCTACGTCAAAAAGGGTG |
| | | | GAGCCAAGAAAAGACCCGCCCCCAGTGACGCAGATAT |
| | | | AAGTGAGCCCAAACGGGTGCGCGAGTCAGTTGCGCA |
| | | | GCCATCGACGTCAGACGCGGAAGCTTCGATCAACTAC |
| | | | GCAGACAGGTACCAAAACAAATGTTCTCGTCACGTGG |
| | | | GCATGAATCTGATGCTGTTTCCCTGCAGACAATGCGAG |
| | | | AGAATGAATCAGAATTCAAATATCTGCTTCACTCACGG |
| | | | ACAGAAAGACTGTTTAGAGTGCTTTCCCGTGTCAGAAT |
| | | | CTCAACCCGTTTCTGTCGTCAAAAAGGCGTATCAGAAA |
| | | | CTGTGCTACATTCATCATATCATGGGAAAGGTGCCAGA |
| | | | CGCTTGCACTGCCTGCGATCTGGTCAATGTGGATTTG |
| | | | GATGACTGCATCTTTGAACAATAAATGATTTAAATCA |
| | | | GGTATGGCTGCCGATGGTTATCTTCCAGATTGGCTC |
| | | | GAGGACAACCTTAGTGAAGGAATTCGCGAGTGGTG |
| | | | GGCTTTGAAACCTGGAGCCCCTCAACCCAAGGCAA |
| | | | ATCAACAACATCAAGACAACGCTCGAGGTCTTGTG |
| | | | CTTCCGGGTTACAAATACCTTGGACCCGGCAACGG |
| | | | ACTCGACAAGGGGGAGCCGGTCAACGCAGCAGAC |
| | | | GCGGCGGCCCTCGAGCACGACAAGGCCTACGACCA |
| | | | GCAGCTCAAGGCCGGAGACAACCCGTACCTCAAGT |
| | | | ACAACCACGCCGACGCCGAGTTCCAGGAGCGGCTC |
| | | | AAAGAAGATACGTCTTTTGGGGGCAACCTCGGGCG |
| | | | AGCAGTCTTCCAGGCCAAAAAGAGGCTTCTTGAAC |
| | | | CTCTTGGTCTGGTTGAGGAAGCGGCTAAGACGGCT |
| | | | CCTGGAAAGAAGAGGCCTGTAGAGCAGTCTCCTCA |
| | | | GGAACCGGACTCCTCCGCGGGTATTGGCAAATCGG |
| | | | GTGCACAGCCCGCTAAAAAGAGACTCAATTTCGGT |
| | | | CAGACTGGCGACACAGAGTCAGTCCCAGACCCTCA |
| | | | ACCAATCGGAGAACCTCCCGCAGCCCCCTCAGGTG |
| | | | TGGGATCTCTTACAATGGCTTCAGGTGGTGGCGCA |
| | | | CCAGTGGCAGACAATAACGAAGGTGCCGATGGAGT |
| | | | GGGTAGTTCCTCGGGAAATTGGCATTGCGATTCCC |
| | | | AATGGCTGGGGGACAGAGTCATCACCACCAGCACC |
| | | | CGAACCTGGGCCCTGCCCACCTACAACAATCACCT |
| | | | CTACAAGCAAATCTCCAACAGCACATCTGGAGGAT |
| | | | CTTCAAATGACAACGCCTACTTCGGCTACAGCACC |
| | | | CCCTGGGGGTATTTTGACTTCAACAGATTCCACTGC |
| | | | CACTTCTCACCACGTGACTGGCAGCGACTCATCAA |
| | | | CAACAACTGGGGATTCCGGCCTAAGCGACTCAACT |
| | | | TCAAGCTCTTCAACATTCAGGTCAAAGAGGTTACG |
| | | | GACAACAATGGAGTCAAGACCATCGCCAATAACCT |
| | | | TACCAGCACGGTCCAGGTCTTCACGGACTCAGACT |
| | | | ATCAGCTCCCGTACGTGCTCGGGTCGGCTCACGAG |
| | | | GGCTGCCTCCCGCCGTTCCCAGCGGACGTTTTCATG |
| | | | ATTCCTCAGTACGGGTATCTGACGCTTAATGATGG |
| | | | AAGCCAGGCCGTGGGTCGTTCGTCCTTTTACTGCCT |
| | | | GGAATATTTCCCGTCGCAAATGCTAAGAACGGGTA |
| | | | ACAACTTCCAGTTCAGCTACGAGTTTGAGAACGTA |
| | | | CCTTTCCATAGCAGCTACGCTCACAGCCAAAGCCT |
| | | | GGACCGACTAATGAATCCACTCATCGACCAATACT |
| | | | TGTACTATCTCTCAAAGACTATTAACGGTTCTGGAC |
| | | | AGAATCAACAAACGCTAAAATTCAGTGTGGCCGGA |
| | | | CCCAGCAACATGGCTGTCCAGGGAAGAAACTACAT |
| | | | ACCTGGACCCAGCTACCGACAACAACGTGTCTCAA |
| | | | CCACTGTGACTCAAAACAACAACAGCGAATTTGCT |
| | | | TGGCCTGGAGCTTCTTCTTGGGCTCTCAATGGACGT |
| | | | AATAGCTTGATGAATCCTGGACCTGCTATGGCCAG |
| | | | CCACAAAGAAGGAGAGGACCGTTTCTTTCCTTTGT |
| | | | CTGGATCTTTAATTTTTGGCAAACAAGGAACTGGA |
| | | | AGAGACAACGTGGATGCGGACAAAGTCATGATAA |
| | | | CCAACGAAGAAGAAATTAAAACTACTAACCCGGTA |
| | | | GCAACGGAGTCCTATGGACAAGTGGCCACAAACCA |
| | | | CCAGAGTGCCCAAGCACAGGCGCAGACCGGCTGG |
| | | | GTTCAAAACCAAGGAATACTTCCGGGTATGGTTTG |
| | | | GCAGGACAGAGATGTGTACCTGCAAGGACCCATTT |
| | | | GGGCCAAAATTCCTCACACGGACGGCAACTTTCAC |
| | | | CCTTCTCCGCTGATGGGAGGGTTTGGAATGAAGCA |
| | | | CCCGCCTCCTCAGATCCTCATCAAAAACACACCTGT |
| | | | ACCTGCGGATCCTCCAACGGCCTTCAACAAGGACA |
| | | | AGCTGAACTCTTTCATCACCCAGTATTCTACTGGCC |
| | | | AAGTCAGCGTGGAGATCGAGTGGGAGCTGCAGAA |
| | | | GGAAAACAGCAAGCGCTGGAACCCGGAGATCCAG |
| | | | TACACTTCCAACTATTACAAGTCTAATAATGTTGAA |
| | | | TTTGCTGTTAATACTGAAGGTGTATATAGTGAACCC |
| | | | CGCCCCATTGGCACCAGATACCTGACTCGTAATCT |
| | | | GTAATTGCTTGTTAATCAATAAACCGTTTAATTC |
| | | | GTTTCAGTTGAACTTTGGTCTCTGCGGAGGTCCT |
| | | | GTATTAGAGGTCACGTGAGTGTTTTGCGACATT |
| | | | TTGCGACACCATGTGGTCACGCTGGGTATTTAA |
| | | | GCCCGAGTGAGCACGCAGGGTCTCCATTTTGAA |
| | | | GCGGGAGGTTTGAAATCCCAATGGCGCGCCGAGC |
| | | | TTGGCTCGAGCATGGTCATAGCTGTTTCCTGTGTGA |
| | | | AATTGTTATCCGCTCACAATTCCACACAACATACG |
| | | | AGCCGGAAGCATAAAGTGTAAAGCCTGGGGTGCCT |
| | | | AATGAGTGAGCTAACTCACATTAATTGCGTTGCGC |
| | | | TCACTGCCCGCTTTCCAGTCGGGAAACCTGTCGTGC |
| | | | CAGCTGCATTAATGAATCGGCCAACGCGCGGGGAG |
| | | | AGGCGGTTTGCGTATTGGGCGCTCTTCCGCTTCCTC |
| | | | GCTCACTGACTCGCTGCGCTCGGTCGTTCGGCTGCG |
| | | | GCGAGCGGTATCAGCTCACTCAAAGGCGGTAATAC |
| | | | GGTTATCCACAGAATCAGGGGATAACGCAGGAAA |
| | | | GAACATGTGAGCAAAAGGCCAGCAAAAGGCCAGG |
| | | | AACCGTAAAAAGGCCGCGTTGCTGGCGTTTTTCCA |
| | | | TAGGCTCCGCCCCCCTGACGAGCATCACAAAAATC |
| | | | GACGCTCAAGTCAGAGGTGGCGAAACCCGACAGG |
| | | | ACTATAAAGATACCAGGCGTTTCCCCCTGGAAGCT |
| | | | CCCTCGTGCGCTCTCCTGTTCCGACCCTGCCGCTTA |
| | | | CCGGATACCTGTCCGCCTTTCTCCCTTCGGGAAGCG |
| | | | TGGCGCTTTCTCATAGCTCACGCTGTAGGTATCTCA |
| | | | GTTCGGTGTAGGTCGTTCGCTCCAAGCTGGGCTGTG |
| | | | TGCACGAACCCCCCGTTCAGCCCGACCGCTGCGCC |
| | | | TTATCCGGTAACTATCGTCTTGAGTCCAACCCGGTA |
| | | | AGACACGACTTATCGCCACTGGCAGCAGCCACTGG |
| | | | TAACAGGATTAGCAGAGCGAGGTATGTAGGCGGTG |
| | | | CTACAGAGTTCTTGAAGTGGTGGCCTAACTACGGC |
| | | | TACACTAGAAGAACAGTATTTGGTATCTGCGCTCT |
| | | | GCTGAAGCCAGTTACCTTCGGAAAAAGAGTTGGTA |
| | | | GCTCTTGATCCGGCAAACAAACCACCGCTGGTAGC |
| | | | GGTGGTTTTTTTGTTTGCAAGCAGCAGATTACGCGC |
| | | | AGAAAAAAAGGATCTCAAGAAGATCCTTTGATCTT |
| | | | TTCTACGGGGTCTGACGCTCAGTGGAACGAAAACT |
| | | | CACGTTAAGGGATTTTGGTCATGAGATTATCAAAA |
| | | | AGGATCTTCACCTAGATCCTTTTAAATTAAAAATGA |
| | | | AGTTTTAAATCAATCTAAAGTATATATGAGTAAAC |
| | | | TTGGTCTGACAGTTAGAAAAACTCATCGAGCATCA |
| | | | AATGAAACTGCAATTTATTCATATCAGGATTATCA |
| | | | ATACCATATTTTTGAAAAAGCCGTTTCTGTAATGAA |
| | | | GGAGAAAACTCACCGAGGCAGTTCCATAGGATGGC |
| | | | AAGATCCTGGTATCGGTCTGCGATTCCGACTCGTCC |
| | | | AACATCAATACAACCTATTAATTTCCCCTCGTCAAA |
| | | | AATAAGGTTATCAAGTGAGAAATCACCATGAGTGA |
| | | | CGACTGAATCCGGTGAGAATGGCAAAAGTTTATGC |
| | | | ATTTCTTTCCAGACTTGTTCAACAGGCCAGCCATTA |
| | | | CGCTCGTCATCAAAATCACTCGCATCAACCAAACC |
| | | | GTTATTCATTCGTGATTGCGCCTGAGCGAGACGAA |
| | | | ATACGCGATCGCTGTTAAAAGGACAATTACAAACA |
| | | | GGAATCGAATGCAACCGGCGCAGGAACACTGCCA |
| | | | GCGCATCAACAATATTTTCACCTGAATCAGGATATT |
| | | | CTTCTAATACCTGGAATGCTGTTTTCCCAGGGATCG |
| | | | CAGTGGTGAGTAACCATGCATCATCAGGAGTACGG |
| | | | ATAAAATGCTTGATGGTCGGAAGAGGCATAAATTC |
| | | | CGTCAGCCAGTTTAGTCTGACCATCTCATCTGTAAC |
| | | | ATCATTGGCAACGCTACCTTTGCCATGTTTCAGAAA |
| | | | CAACTCTGGCGCATCGGGCTTCCCATACAATCGAT |
| | | | AGATTGTCGCACCTGATTGCCCGACATTATCGCGA |
| | | | GCCCATTTATACCCATATAAATCAGCATCCATGTTG |
| | | | GAATTTAATCGCGGCCTAGAGCAAGACGTTTCCCG |
| | | | TTGAATATGGCTCATACTCTTCCTTTTTCAATATTAT |
| | | | TGAAGCATTTATCAGGGTTATTGTCTCATGAGCGG |
| | | | ATACATATTTGAATGTATTTAGAAAAATAAACAAA |
| | | | TAGGGGTTCCGCGCACATTTCCCCGAAAAGTGCCA |
| | | | CCTGACGTCTAAGAAACCATTATTATCATGACATTA |
| | | | ACCTATAAAAATAGGCGTATCACGAGGCCCTTTCG |
| | | | TC |
| 47 | BSG098 | Rep2.Cap9-p5 | TCGCGCGTTTCGGTGATGACGGTGAAAACCTCTGA |
| | | | CACATGCAGCTCCCGGAGACGGTCACAGCTTGTCT |
| | | | GTAAGCGGATGCCGGGAGCAGACAAGCCCGTCAG |
| | | | GGCGCGTCAGCGGGTGTTGGCGGGTGTCGGGGCTG |
| | | | GCTTAACTATGCGGCATCAGAGCAGATTGTACTGA |
| | | | GAGTGCACCATATGCGGTGTGAAATACCGCACAGA |
| | | | TGCGTAAGGAGAAAATACCGCATCAGGCGCCATTC |
| | | | GCCATTCAGGCTGCGCAACTGTTGGGAAGGGCGAT |
| | | | CGGTGCGGGCCTCTTCGCTATTACGCCAGCTGGCG |
| | | | AAAGGGGGATGTGCTGCAAGGCGATTAAGTTGGGT |
| | | | AACGCCAGGGTTTTCCCAGTCACGACGTTGTAAAA |
| | | | CGACGGCCAGTGAATTGACGCGTATTGGGATCGCG |
| | | | CAGCCGCCATGCCGGGGTTTTACGAGATTGTGATTAA |
| | | | GGTCCCCAGCGACCTTGACGAGCATCTGCCCGGCATT |
| | | | TCTGACAGCTTTGTGAACTGGGTGGCCGAGAAGGAAT |
| | | | GGGAGTTGCCGCCAGATTCTGACATGGATCTGAATCT |
| | | | GATTGAGCAGGCACCCCTGACCGTGGCCGAGAAGCT |
| | | | GCAGCGCGACTTTCTGACGGAATGGCGCCGTGTGAGT |
| | | | AAGGCCCCGGAGGCTCTTTTCTTTGTGCAATTTGAGAA |
| | | | GGGAGAGAGCTACTTCCACATGCACGTGCTCGTGGAA |
| | | | ACCACCGGGGTGAAATCCATGGTTTTGGGACGTTTCCT |
| | | | GAGTCAGATTCGCGAAAAACTGATTCAGAGAATTTACC |
| | | | GCGGGATCGAGCCGACTTTGCCAAACTGGTTCGCGGT |
| | | | CACAAAGACCAGAAATGGCGCCGGAGGCGGGAACAA |
| | | | GGTGGTGGATGAGTGCTACATCCCCAATTACTTGCTCC |
| | | | CCAAAACCCAGCCTGAGCTCCAGTGGGCGTGGACTAA |
| | | | TATGGAACAGTATTTAAGCGCCTGTTTGAATCTCACGG |
| | | | AGCGTAAACGGTTGGTGGCGCAGCATCTGACGCACGT |
| | | | GTCGCAGACGCAGGAGCAGAACAAAGAGAATCAGAAT |
| | | | CCCAATTCTGATGCGCCGGTGATCAGATCAAAAACTTC |
| | | | AGCCAGGTACATGGAGCTGGTCGGGTGGCTCGTGGA |
| | | | CAAGGGGATTACCTCGGAGAAGCAGTGGATCCAGGAG |
| | | | GACCAGGCCTCATACATCTCCTTCAATGCGGCCTCCAA |
| | | | CTCGCGGTCCCAAATCAAGGCTGCCTTGGACAATGCG |
| | | | GGAAAGATTATGAGCCTGACTAAAACCGCCCCCGACT |
| | | | ACCTGGTGGGCCAGCAGCCCGTGGAGGACATTTCCAG |
| | | | CAATCGGATTTATAAAATTTTGGAACTAAACGGGTACG |
| | | | ATCCCCAATATGCGGCTTCCGTCTTTCTGGGATGGGC |
| | | | CACGAAAAAGTTCGGCAAGAGGAACACCATCTGGCTG |
| | | | TTTGGGCCTGCAACTACCGGGAAGACCAACATCGCGG |
| | | | AGGCCATAGCCCACACTGTGCCCTTCTACGGGTGCGT |
| | | | AAACTGGACCAATGAGAACTTTCCCTTCAACGACTGTG |
| | | | TCGACAAGATGGTGATCTGGTGGGAGGAGGGGAAGAT |
| | | | GACCGCCAAGGTCGTGGAGTCGGCCAAAGCCATTCTC |
| | | | GGAGGAAGCAAGGTGCGCGTGGACCAGAAATGCAAG |
| | | | TCCTCGGCCCAGATAGACCCGACTCCCGTGATCGTCA |
| | | | CCTCCAACACCAACATGTGCGCCGTGATTGACGGGAA |
| | | | CTCAACGACCTTCGAACACCAGCAGCCGTTGCAAGAC |
| | | | CGGATGTTCAAATTTGAACTCACCCGCCGTCTGGATCA |
| | | | TGACTTTGGGAAGGTCACCAAGCAGGAAGTCAAAGAC |
| | | | TTTTTCCGGTGGGCAAAGGATCACGTGGTTGAGGTGG |
| | | | AGCATGAATTCTACGTCAAAAAGGGTGGAGCCAAGAAA |
| | | | AGACCCGCCCCCAGTGACGCAGATATAAGTGAGCCCA |
| | | | AACGGGTGCGCGAGTCAGTTGCGCAGCCATCGACGTC |
| | | | AGACGCGGAAGCTTCGATCAACTACGCAGACAGGTAC |
| | | | CAAAACAAATGTTCTCGTCACGTGGGCATGAATCTGAT |
| | | | GCTGTTTCCCTGCAGACAATGCGAGAGAATGAATCAGA |
| | | | ATTCAAATATCTGCTTCACTCACGGACAGAAAGACTGT |
| | | | TTAGAGTGCTTTCCCGTGTCAGAATCTCAACCCGTTTC |
| | | | TGTCGTCAAAAAGGCGTATCAGAAACTGTGCTACATTC |
| | | | ATCATATCATGGGAAAGGTGCCAGACGCTTGCACTGC |
| | | | CTGCGATCTGGTCAATGTGGATTTGGATGACTGCATCT |
| | | | TTGAACAATAAATGATTTAAATCAGGTATGGCTGCC |
| | | | GATGGTTATCTTCCAGATTGGCTCGAGGACAACCTT |
| | | | AGTGAAGGAATTCGCGAGTGGTGGGCTTTGAAACC |
| | | | TGGAGCCCCTCAACCCAAGGCAAATCAACAACATC |
| | | | AAGACAACGCTCGAGGTCTTGTGCTTCCGGGTTAC |
| | | | AAATACCTTGGACCCGGCAACGGACTCGACAAGGG |
| | | | GGAGCCGGTCAACGCAGCAGACGCGGCGGCCCTCG |
| | | | AGCACGACAAGGCCTACGACCAGCAGCTCAAGGCC |
| | | | GGAGACAACCCGTACCTCAAGTACAACCACGCCGA |
| | | | CGCCGAGTTCCAGGAGCGGCTCAAAGAAGATACGT |
| | | | CTTTTGGGGGCAACCTCGGGCGAGCAGTCTTCCAG |
| | | | GCCAAAAAGAGGCTTCTTGAACCTCTTGGTCTGGTT |
| | | | GAGGAAGCGGCTAAGACGGCTCCTGGAAAGAAGA |
| | | | GGCCTGTAGAGCAGTCTCCTCAGGAACCGGACTCC |
| | | | TCCGCGGGTATTGGCAAATCGGGTGCACAGCCCGC |
| | | | TAAAAAGAGACTCAATTTCGGTCAGACTGGCGACA |
| | | | CAGAGTCAGTCCCAGACCCTCAACCAATCGGAGAA |
| | | | CCTCCCGCAGCCCCCTCAGGTGTGGGATCTCTTACA |
| | | | ATGGCTTCAGGTGGTGGCGCACCAGTGGCAGACAA |
| | | | TAACGAAGGTGCCGATGGAGTGGGTAGTTCCTCGG |
| | | | GAAATTGGCATTGCGATTCCCAATGGCTGGGGGAC |
| | | | AGAGTCATCACCACCAGCACCCGAACCTGGGCCCT |
| | | | GCCCACCTACAACAATCACCTCTACAAGCAAATCT |
| | | | CCAACAGCACATCTGGAGGATCTTCAAATGACAAC |
| | | | GCCTACTTCGGCTACAGCACCCCCTGGGGGTATTTT |
| | | | GACTTCAACAGATTCCACTGCCACTTCTCACCACGT |
| | | | GACTGGCAGCGACTCATCAACAACAACTGGGGATT |
| | | | CCGGCCTAAGCGACTCAACTTCAAGCTCTTCAACA |
| | | | TTCAGGTCAAAGAGGTTACGGACAACAATGGAGTC |
| | | | AAGACCATCGCCAATAACCTTACCAGCACGGTCCA |
| | | | GGTCTTCACGGACTCAGACTATCAGCTCCCGTACGT |
| | | | GCTCGGGTCGGCTCACGAGGGCTGCCTCCCGCCGT |
| | | | TCCCAGCGGACGTTTTCATGATTCCTCAGTACGGGT |
| | | | ATCTGACGCTTAATGATGGAAGCCAGGCCGTGGGT |
| | | | CGTTCGTCCTTTTACTGCCTGGAATATTTCCCGTCG |
| | | | CAAATGCTAAGAACGGGTAACAACTTCCAGTTCAG |
| | | | CTACGAGTTTGAGAACGTACCTTTCCATAGCAGCT |
| | | | ACGCTCACAGCCAAAGCCTGGACCGACTAATGAAT |
| | | | CCACTCATCGACCAATACTTGTACTATCTCTCAAAG |
| | | | ACTATTAACGGTTCTGGACAGAATCAACAAACGCT |
| | | | AAAATTCAGTGTGGCCGGACCCAGCAACATGGCTG |
| | | | TCCAGGGAAGAAACTACATACCTGGACCCAGCTAC |
| | | | CGACAACAACGTGTCTCAACCACTGTGACTCAAAA |
| | | | CAACAACAGCGAATTTGCTTGGCCTGGAGCTTCTTC |
| | | | TTGGGCTCTCAATGGACGTAATAGCTTGATGAATC |
| | | | CTGGACCTGCTATGGCCAGCCACAAAGAAGGAGAG |
| | | | GACCGTTTCTTTCCTTTGTCTGGATCTTTAATTTTTG |
| | | | GCAAACAAGGAACTGGAAGAGACAACGTGGATGC |
| | | | GGACAAAGTCATGATAACCAACGAAGAAGAAATT |
| | | | AAAACTACTAACCCGGTAGCAACGGAGTCCTATGG |
| | | | ACAAGTGGCCACAAACCACCAGAGTGCCCAAGCAC |
| | | | AGGCGCAGACCGGCTGGGTTCAAAACCAAGGAAT |
| | | | ACTTCCGGGTATGGTTTGGCAGGACAGAGATGTGT |
| | | | ACCTGCAAGGACCCATTTGGGCCAAAATTCCTCAC |
| | | | ACGGACGGCAACTTTCACCCTTCTCCGCTGATGGG |
| | | | AGGGTTTGGAATGAAGCACCCGCCTCCTCAGATCC |
| | | | TCATCAAAAACACACCTGTACCTGCGGATCCTCCA |
| | | | ACGGCCTTCAACAAGGACAAGCTGAACTCTTTCAT |
| | | | CACCCAGTATTCTACTGGCCAAGTCAGCGTGGAGA |
| | | | TCGAGTGGGAGCTGCAGAAGGAAAACAGCAAGCG |
| | | | CTGGAACCCGGAGATCCAGTACACTTCCAACTATT |
| | | | ACAAGTCTAATAATGTTGAATTTGCTGTTAATACTG |
| | | | AAGGTGTATATAGTGAACCCCGCCCCATTGGCACC |
| | | | AGATACCTGACTCGTAATCTGTAATTGCTTGTTAA |
| | | | TCAATAAACCGTTTAATTCGTTTCAGTTGAACTT |
| | | | TGGTCTCTGCGGAGGTCCTGTATTAGAGGTCACG |
| | | | TGAGTGTTTTGCGACATTTTGCGACACCATGTG |
| | | | GTCACGCTGGGTATTTAAGCCCGAGTGAGCACG |
| | | | CAGGGTCTCCATTTTGAAGCGGGAGGTTTGAAA |
| | | | TCCCAATGGCGCGCCGAGCTTGGCTCGAGCATGGT |
| | | | CATAGCTGTTTCCTGTGTGAAATTGTTATCCGCTCA |
| | | | CAATTCCACACAACATACGAGCCGGAAGCATAAAG |
| | | | TGTAAAGCCTGGGGTGCCTAATGAGTGAGCTAACT |
| | | | CACATTAATTGCGTTGCGCTCACTGCCCGCTTTCCA |
| | | | GTCGGGAAACCTGTCGTGCCAGCTGCATTAATGAA |
| | | | TCGGCCAACGCGCGGGGAGAGGCGGTTTGCGTATT |
| | | | GGGCGCTCTTCCGCTTCCTCGCTCACTGACTCGCTG |
| | | | CGCTCGGTCGTTCGGCTGCGGCGAGCGGTATCAGC |
| | | | TCACTCAAAGGCGGTAATACGGTTATCCACAGAAT |
| | | | CAGGGGATAACGCAGGAAAGAACATGTGAGCAAA |
| | | | AGGCCAGCAAAAGGCCAGGAACCGTAAAAAGGCC |
| | | | GCGTTGCTGGCGTTTTTCCATAGGCTCCGCCCCCCT |
| | | | GACGAGCATCACAAAAATCGACGCTCAAGTCAGAG |
| | | | GTGGCGAAACCCGACAGGACTATAAAGATACCAG |
| | | | GCGTTTCCCCCTGGAAGCTCCCTCGTGCGCTCTCCT |
| | | | GTTCCGACCCTGCCGCTTACCGGATACCTGTCCGCC |
| | | | TTTCTCCCTTCGGGAAGCGTGGCGCTTTCTCATAGC |
| | | | TCACGCTGTAGGTATCTCAGTTCGGTGTAGGTCGTT |
| | | | CGCTCCAAGCTGGGCTGTGTGCACGAACCCCCCGT |
| | | | TCAGCCCGACCGCTGCGCCTTATCCGGTAACTATCG |
| | | | TCTTGAGTCCAACCCGGTAAGACACGACTTATCGC |
| | | | CACTGGCAGCAGCCACTGGTAACAGGATTAGCAGA |
| | | | GCGAGGTATGTAGGCGGTGCTACAGAGTTCTTGAA |
| | | | GTGGTGGCCTAACTACGGCTACACTAGAAGAACAG |
| | | | TATTTGGTATCTGCGCTCTGCTGAAGCCAGTTACCT |
| | | | TCGGAAAAAGAGTTGGTAGCTCTTGATCCGGCAAA |
| | | | CAAACCACCGCTGGTAGCGGTGGTTTTTTTGTTTGC |
| | | | AAGCAGCAGATTACGCGCAGAAAAAAAGGATCTC |
| | | | AAGAAGATCCTTTGATCTTTTCTACGGGGTCTGACG |
| | | | CTCAGTGGAACGAAAACTCACGTTAAGGGATTTTG |
| | | | GTCATGAGATTATCAAAAAGGATCTTCACCTAGAT |
| | | | CCTTTTAAATTAAAAATGAAGTTTTAAATCAATCTA |
| | | | AAGTATATATGAGTAAACTTGGTCTGACAGTTAGA |
| | | | AAAACTCATCGAGCATCAAATGAAACTGCAATTTA |
| | | | TTCATATCAGGATTATCAATACCATATTTTTGAAAA |
| | | | AGCCGTTTCTGTAATGAAGGAGAAAACTCACCGAG |
| | | | GCAGTTCCATAGGATGGCAAGATCCTGGTATCGGT |
| | | | CTGCGATTCCGACTCGTCCAACATCAATACAACCT |
| | | | ATTAATTTCCCCTCGTCAAAAATAAGGTTATCAAGT |
| | | | GAGAAATCACCATGAGTGACGACTGAATCCGGTGA |
| | | | GAATGGCAAAAGTTTATGCATTTCTTTCCAGACTTG |
| | | | TTCAACAGGCCAGCCATTACGCTCGTCATCAAAAT |
| | | | CACTCGCATCAACCAAACCGTTATTCATTCGTGATT |
| | | | GCGCCTGAGCGAGACGAAATACGCGATCGCTGTTA |
| | | | AAAGGACAATTACAAACAGGAATCGAATGCAACC |
| | | | GGCGCAGGAACACTGCCAGCGCATCAACAATATTT |
| | | | TCACCTGAATCAGGATATTCTTCTAATACCTGGAAT |
| | | | GCTGTTTTCCCAGGGATCGCAGTGGTGAGTAACCA |
| | | | TGCATCATCAGGAGTACGGATAAAATGCTTGATGG |
| | | | TCGGAAGAGGCATAAATTCCGTCAGCCAGTTTAGT |
| | | | CTGACCATCTCATCTGTAACATCATTGGCAACGCTA |
| | | | CCTTTGCCATGTTTCAGAAACAACTCTGGCGCATCG |
| | | | GGCTTCCCATACAATCGATAGATTGTCGCACCTGAT |
| | | | TGCCCGACATTATCGCGAGCCCATTTATACCCATAT |
| | | | AAATCAGCATCCATGTTGGAATTTAATCGCGGCCT |
| | | | AGAGCAAGACGTTTCCCGTTGAATATGGCTCATAC |
| | | | TCTTCCTTTTTCAATATTATTGAAGCATTTATCAGG |
| | | | GTTATTGTCTCATGAGCGGATACATATTTGAATGTA |
| | | | TTTAGAAAAATAAACAAATAGGGGTTCCGCGCACA |
| | | | TTTCCCCGAAAAGTGCCACCTGACGTCTAAGAAAC |
| | | | CATTATTATCATGACATTAACCTATAAAAATAGGC |
| | | | GTATCACGAGGCCCTTTCGTC |
| 48 | BSG171 | P5- | TCGCGCGTTTCGGTGATGACGGTGAAAACCTCTGA |
| | | AltRep2.Cap9- | CACATGCAGCTCCCGGAGACGGTCACAGCTTGTCT |
| | | p5 | GTAAGCGGATGCCGGGAGCAGACAAGCCCGTCAG |
| | | | GGCGCGTCAGCGGGTGTTGGCGGGTGTCGGGGCTG |
| | | | GCTTAACTATGCGGCATCAGAGCAGATTGTACTGA |
| | | | GAGTGCACCATATGCGGTGTGAAATACCGCACAGA |
| | | | TGCGTAAGGAGAAAATACCGCATCAGGCGCCATTC |
| | | | GCCATTCAGGCTGCGCAACTGTTGGGAAGGGCGAT |
| | | | CGGTGCGGGCCTCTTCGCTATTACGCCAGCTGGCG |
| | | | AAAGGGGGATGTGCTGCAAGGCGATTAAGTTGGGT |
| | | | AACGCCAGGGTTTTCCCAGTCACGACGTTGTAAAA |
| | | | CGACGGCCAGTGAATTGACGCGTATTGGGATGAGG |
| | | | TCCTGTATTAGAGGTCACGTGAGTGTTTTGCGA |
| | | | CATTTTGCGACACCATGTGGTCACGCTGGGTAT |
| | | | TTAAGCCCGAGTGAGCACGCAGGGTCTCCATTT |
| | | | TGAAGCGGGAGGTTTGAACGCGCAGCCGCCACGC |
| | | | CGGGGTTTTACGAGATTGTGATTAAGGTCCCCAGCGA |
| | | | CCTTGACGAGCATCTGCCCGGCATTTCTGACAGCTTTG |
| | | | TGAACTGGGTGGCCGAGAAGGAATGGGAGTTGCCGC |
| | | | CAGATTCTGACATGGATCTGAATCTGATTGAGCAGGCA |
| | | | CCCCTGACCGTGGCCGAGAAGCTGCAGCGCGACTTTC |
| | | | TGACGGAATGGCGCCGTGTGAGTAAGGCCCCGGAGG |
| | | | CTCTTTTCTTTGTGCAATTTGAGAAGGGAGAGAGCTAC |
| | | | TTCCACATGCACGTGCTCGTGGAAACCACCGGGGTGA |
| | | | AATCCATGGTTTTGGGACGTTTCCTGAGTCAGATTCGC |
| | | | GAAAAACTGATTCAGAGAATTTACCGCGGGATCGAGC |
| | | | CGACTTTGCCAAACTGGTTCGCGGTCACAAAGACCAG |
| | | | AAATGGCGCCGGAGGCGGGAACAAGGTGGTGGATGA |
| | | | GTGCTACATCCCCAATTACTTGCTCCCCAAAACCCAGC |
| | | | CTGAGCTCCAGTGGGCGTGGACTAATATGGAACAGTA |
| | | | TTTAAGCGCCTGTTTGAATCTCACGGAGCGTAAACGGT |
| | | | TGGTGGCGCAGCATCTGACGCACGTGTCGCAGACGCA |
| | | | GGAGCAGAACAAAGAGAATCAGAATCCCAATTCTGATG |
| | | | CGCCGGTGATCAGATCAAAAACTTCAGCCAGGTACAT |
| | | | GGAGCTGGTCGGGTGGCTCGTGGACAAGGGGATTAC |
| | | | CTCGGAGAAGCAGTGGATCCAGGAGGACCAGGCCTC |
| | | | ATACATCTCCTTCAATGCGGCCTCCAACTCGCGGTCCC |
| | | | AAATCAAGGCTGCCTTGGACAATGCGGGAAAGATTATG |
| | | | AGCCTGACTAAAACCGCCCCCGACTACCTGGTGGGCC |
| | | | AGCAGCCCGTGGAGGACATTTCCAGCAATCGGATTTAT |
| | | | AAAATTTTGGAACTAAACGGGTACGATCCCCAATATGC |
| | | | GGCTTCCGTCTTTCTGGGATGGGCCACGAAAAAGTTC |
| | | | GGCAAGAGGAACACCATCTGGCTGTTTGGGCCTGCAA |
| | | | CTACCGGGAAGACCAACATCGCGGAGGCCATAGCCCA |
| | | | CACTGTGCCCTTCTACGGGTGCGTAAACTGGACCAAT |
| | | | GAGAACTTTCCCTTCAACGACTGTGTCGACAAGATGGT |
| | | | GATCTGGTGGGAGGAGGGGAAGATGACCGCCAAGGT |
| | | | CGTGGAGTCGGCCAAAGCCATTCTCGGAGGAAGCAAG |
| | | | GTGCGCGTGGACCAGAAATGCAAGTCCTCGGCCCAGA |
| | | | TAGACCCGACTCCCGTGATCGTCACCTCCAACACCAA |
| | | | CATGTGCGCCGTGATTGACGGGAACTCAACGACCTTC |
| | | | GAACACCAGCAGCCGTTGCAAGACCGGATGTTCAAAT |
| | | | TTGAACTCACCCGCCGTCTGGATCATGACTTTGGGAAG |
| | | | GTCACCAAGCAGGAAGTCAAAGACTTTTTCCGGTGGG |
| | | | CAAAGGATCACGTGGTTGAGGTGGAGCATGAATTCTA |
| | | | CGTCAAAAAGGGTGGAGCCAAGAAAAGACCCGCCCCC |
| | | | AGTGACGCAGATATAAGTGAGCCCAAACGGGTGCGCG |
| | | | AGTCAGTTGCGCAGCCATCGACGTCAGACGCGGAAGC |
| | | | TTCGATCAACTACGCAGACAGGTACCAAAACAAATGTT |
| | | | CTCGTCACGTGGGCATGAATCTGATGCTGTTTCCCTGC |
| | | | AGACAATGCGAGAGAATGAATCAGAATTCAAATATCTG |
| | | | CTTCACTCACGGACAGAAAGACTGTTTAGAGTGCTTTC |
| | | | CCGTGTCAGAATCTCAACCCGTTTCTGTCGTCAAAAAG |
| | | | GCGTATCAGAAACTGTGCTACATTCATCATATCATGGG |
| | | | AAAGGTGCCAGACGCTTGCACTGCCTGCGATCTGGTC |
| | | | AATGTGGATTTGGATGACTGCATCTTTGAACAATAAAT |
| | | | GATTTAAATCAGGTATGGCTGCCGATGGTTATCTTC |
| | | | CAGATTGGCTCGAGGACAACCTTAGTGAAGGAATT |
| | | | CGCGAGTGGTGGGCTTTGAAACCTGGAGCCCCTCA |
| | | | ACCCAAGGCAAATCAACAACATCAAGACAACGCTC |
| | | | GAGGTCTTGTGCTTCCGGGTTACAAATACCTTGGAC |
| | | | CCGGCAACGGACTCGACAAGGGGGAGCCGGTCAA |
| | | | CGCAGCAGACGCGGCGGCCCTCGAGCACGACAAG |
| | | | GCCTACGACCAGCAGCTCAAGGCCGGAGACAACCC |
| | | | GTACCTCAAGTACAACCACGCCGACGCCGAGTTCC |
| | | | AGGAGCGGCTCAAAGAAGATACGTCTTTTGGGGGC |
| | | | AACCTCGGGCGAGCAGTCTTCCAGGCCAAAAAGAG |
| | | | GCTTCTTGAACCTCTTGGTCTGGTTGAGGAAGCGGC |
| | | | TAAGACGGCTCCTGGAAAGAAGAGGCCTGTAGAGC |
| | | | AGTCTCCTCAGGAACCGGACTCCTCCGCGGGTATT |
| | | | GGCAAATCGGGTGCACAGCCCGCTAAAAAGAGACT |
| | | | CAATTTCGGTCAGACTGGCGACACAGAGTCAGTCC |
| | | | CAGACCCTCAACCAATCGGAGAACCTCCCGCAGCC |
| | | | CCCTCAGGTGTGGGATCTCTTACAATGGCTTCAGGT |
| | | | GGTGGCGCACCAGTGGCAGACAATAACGAAGGTG |
| | | | CCGATGGAGTGGGTAGTTCCTCGGGAAATTGGCAT |
| | | | TGCGATTCCCAATGGCTGGGGGACAGAGTCATCAC |
| | | | CACCAGCACCCGAACCTGGGCCCTGCCCACCTACA |
| | | | ACAATCACCTCTACAAGCAAATCTCCAACAGCACA |
| | | | TCTGGAGGATCTTCAAATGACAACGCCTACTTCGG |
| | | | CTACAGCACCCCCTGGGGGTATTTTGACTTCAACA |
| | | | GATTCCACTGCCACTTCTCACCACGTGACTGGCAGC |
| | | | GACTCATCAACAACAACTGGGGATTCCGGCCTAAG |
| | | | CGACTCAACTTCAAGCTCTTCAACATTCAGGTCAA |
| | | | AGAGGTTACGGACAACAATGGAGTCAAGACCATCG |
| | | | CCAATAACCTTACCAGCACGGTCCAGGTCTTCACG |
| | | | GACTCAGACTATCAGCTCCCGTACGTGCTCGGGTC |
| | | | GGCTCACGAGGGCTGCCTCCCGCCGTTCCCAGCGG |
| | | | ACGTTTTCATGATTCCTCAGTACGGGTATCTGACGC |
| | | | TTAATGATGGAAGCCAGGCCGTGGGTCGTTCGTCC |
| | | | TTTTACTGCCTGGAATATTTCCCGTCGCAAATGCTA |
| | | | AGAACGGGTAACAACTTCCAGTTCAGCTACGAGTT |
| | | | TGAGAACGTACCTTTCCATAGCAGCTACGCTCACA |
| | | | GCCAAAGCCTGGACCGACTAATGAATCCACTCATC |
| | | | GACCAATACTTGTACTATCTCTCAAAGACTATTAAC |
| | | | GGTTCTGGACAGAATCAACAAACGCTAAAATTCAG |
| | | | TGTGGCCGGACCCAGCAACATGGCTGTCCAGGGAA |
| | | | GAAACTACATACCTGGACCCAGCTACCGACAACAA |
| | | | CGTGTCTCAACCACTGTGACTCAAAACAACAACAG |
| | | | CGAATTTGCTTGGCCTGGAGCTTCTTCTTGGGCTCT |
| | | | CAATGGACGTAATAGCTTGATGAATCCTGGACCTG |
| | | | CTATGGCCAGCCACAAAGAAGGAGAGGACCGTTTC |
| | | | TTTCCTTTGTCTGGATCTTTAATTTTTGGCAAACAA |
| | | | GGAACTGGAAGAGACAACGTGGATGCGGACAAAG |
| | | | TCATGATAACCAACGAAGAAGAAATTAAAACTACT |
| | | | AACCCGGTAGCAACGGAGTCCTATGGACAAGTGGC |
| | | | CACAAACCACCAGAGTGCCCAAGCACAGGCGCAG |
| | | | ACCGGCTGGGTTCAAAACCAAGGAATACTTCCGGG |
| | | | TATGGTTTGGCAGGACAGAGATGTGTACCTGCAAG |
| | | | GACCCATTTGGGCCAAAATTCCTCACACGGACGGC |
| | | | AACTTTCACCCTTCTCCGCTGATGGGAGGGTTTGGA |
| | | | ATGAAGCACCCGCCTCCTCAGATCCTCATCAAAAA |
| | | | CACACCTGTACCTGCGGATCCTCCAACGGCCTTCA |
| | | | ACAAGGACAAGCTGAACTCTTTCATCACCCAGTAT |
| | | | TCTACTGGCCAAGTCAGCGTGGAGATCGAGTGGGA |
| | | | GCTGCAGAAGGAAAACAGCAAGCGCTGGAACCCG |
| | | | GAGATCCAGTACACTTCCAACTATTACAAGTCTAA |
| | | | TAATGTTGAATTTGCTGTTAATACTGAAGGTGTATA |
| | | | TAGTGAACCCCGCCCCATTGGCACCAGATACCTGA |
| | | | CTCGTAATCTGTAATTGCTTGTTAATCAATAAACC |
| | | | GTTTAATTCGTTTCAGTTGAACTTTGGTCTCTGCG |
| | | | GAGGTCCTGTATTAGAGGTCACGTGAGTGTTTT |
| | | | GCGACATTTTGCGACACCATGTGGTCACGCTGG |
| | | | GTATTTAAGCCCGAGTGAGCACGCAGGGTCTCC |
| | | | ATTTTGAAGCGGGAGGTTTGAAATCCCAATGGCG |
| | | | CGCCGAGCTTGGCTCGAGCATGGTCATAGCTGTTTC |
| | | | CTGTGTGAAATTGTTATCCGCTCACAATTCCACACA |
| | | | ACATACGAGCCGGAAGCATAAAGTGTAAAGCCTGG |
| | | | GGTGCCTAATGAGTGAGCTAACTCACATTAATTGC |
| | | | GTTGCGCTCACTGCCCGCTTTCCAGTCGGGAAACCT |
| | | | GTCGTGCCAGCTGCATTAATGAATCGGCCAACGCG |
| | | | CGGGGAGAGGCGGTTTGCGTATTGGGCGCTCTTCC |
| | | | GCTTCCTCGCTCACTGACTCGCTGCGCTCGGTCGTT |
| | | | CGGCTGCGGCGAGCGGTATCAGCTCACTCAAAGGC |
| | | | GGTAATACGGTTATCCACAGAATCAGGGGATAACG |
| | | | CAGGAAAGAACATGTGAGCAAAAGGCCAGCAAAA |
| | | | GGCCAGGAACCGTAAAAAGGCCGCGTTGCTGGCGT |
| | | | TTTTCCATAGGCTCCGCCCCCCTGACGAGCATCACA |
| | | | AAAATCGACGCTCAAGTCAGAGGTGGCGAAACCCG |
| | | | ACAGGACTATAAAGATACCAGGCGTTTCCCCCTGG |
| | | | AAGCTCCCTCGTGCGCTCTCCTGTTCCGACCCTGCC |
| | | | GCTTACCGGATACCTGTCCGCCTTTCTCCCTTCGGG |
| | | | AAGCGTGGCGCTTTCTCATAGCTCACGCTGTAGGT |
| | | | ATCTCAGTTCGGTGTAGGTCGTTCGCTCCAAGCTGG |
| | | | GCTGTGTGCACGAACCCCCCGTTCAGCCCGACCGC |
| | | | TGCGCCTTATCCGGTAACTATCGTCTTGAGTCCAAC |
| | | | CCGGTAAGACACGACTTATCGCCACTGGCAGCAGC |
| | | | CACTGGTAACAGGATTAGCAGAGCGAGGTATGTAG |
| | | | GCGGTGCTACAGAGTTCTTGAAGTGGTGGCCTAAC |
| | | | TACGGCTACACTAGAAGAACAGTATTTGGTATCTG |
| | | | CGCTCTGCTGAAGCCAGTTACCTTCGGAAAAAGAG |
| | | | TTGGTAGCTCTTGATCCGGCAAACAAACCACCGCT |
| | | | GGTAGCGGTGGTTTTTTTGTTTGCAAGCAGCAGATT |
| | | | ACGCGCAGAAAAAAAGGATCTCAAGAAGATCCTTT |
| | | | GATCTTTTCTACGGGGTCTGACGCTCAGTGGAACG |
| | | | AAAACTCACGTTAAGGGATTTTGGTCATGAGATTA |
| | | | TCAAAAAGGATCTTCACCTAGATCCTTTTAAATTAA |
| | | | AAATGAAGTTTTAAATCAATCTAAAGTATATATGA |
| | | | GTAAACTTGGTCTGACAGTTAGAAAAACTCATCGA |
| | | | GCATCAAATGAAACTGCAATTTATTCATATCAGGA |
| | | | TTATCAATACCATATTTTTGAAAAAGCCGTTTCTGT |
| | | | AATGAAGGAGAAAACTCACCGAGGCAGTTCCATAG |
| | | | GATGGCAAGATCCTGGTATCGGTCTGCGATTCCGA |
| | | | CTCGTCCAACATCAATACAACCTATTAATTTCCCCT |
| | | | CGTCAAAAATAAGGTTATCAAGTGAGAAATCACCA |
| | | | TGAGTGACGACTGAATCCGGTGAGAATGGCAAAAG |
| | | | TTTATGCATTTCTTTCCAGACTTGTTCAACAGGCCA |
| | | | GCCATTACGCTCGTCATCAAAATCACTCGCATCAA |
| | | | CCAAACCGTTATTCATTCGTGATTGCGCCTGAGCGA |
| | | | GACGAAATACGCGATCGCTGTTAAAAGGACAATTA |
| | | | CAAACAGGAATCGAATGCAACCGGCGCAGGAACA |
| | | | CTGCCAGCGCATCAACAATATTTTCACCTGAATCA |
| | | | GGATATTCTTCTAATACCTGGAATGCTGTTTTCCCA |
| | | | GGGATCGCAGTGGTGAGTAACCATGCATCATCAGG |
| | | | AGTACGGATAAAATGCTTGATGGTCGGAAGAGGCA |
| | | | TAAATTCCGTCAGCCAGTTTAGTCTGACCATCTCAT |
| | | | CTGTAACATCATTGGCAACGCTACCTTTGCCATGTT |
| | | | TCAGAAACAACTCTGGCGCATCGGGCTTCCCATAC |
| | | | AATCGATAGATTGTCGCACCTGATTGCCCGACATT |
| | | | ATCGCGAGCCCATTTATACCCATATAAATCAGCAT |
| | | | CCATGTTGGAATTTAATCGCGGCCTAGAGCAAGAC |
| | | | GTTTCCCGTTGAATATGGCTCATACTCTTCCTTTTTC |
| | | | AATATTATTGAAGCATTTATCAGGGTTATTGTCTCA |
| | | | TGAGCGGATACATATTTGAATGTATTTAGAAAAAT |
| | | | AAACAAATAGGGGTTCCGCGCACATTTCCCCGAAA |
| | | | AGTGCCACCTGACGTCTAAGAAACCATTATTATCA |
| | | | TGACATTAACCTATAAAAATAGGCGTATCACGAGG |
| | | | CCCTTTCGTC |
| 49 | AAV2 | | TTGCTTGTTAATCAATAAACCGTTTAATTCGTTTCA |
| | polyA | | GTTGA |
| 52 | pYZZ447 | p5-Rep2.Cap5- | TCGCGCGTTTCGGTGATGACGGTGAAAACCTCTGA |
| | | p5.p5 | CACATGCAGCTCCCGGAGACGGTCACAGCTTGTCT |
| | | | GTAAGCGGATGCCGGGAGCAGACAAGCCCGTCAG |
| | | | GGCGCGTCAGCGGGTGTTGGCGGGTGTCGGGGCTG |
| | | | GCTTAACTATGCGGCATCAGAGCAGATTGTACTGA |
| | | | GAGTGCACCATATGCGGTGTGAAATACCGCACAGA |
| | | | TGCGTAAGGAGAAAATACCGCATCAGGCGCCATTC |
| | | | GCCATTCAGGCTGCGCAACTGTTGGGAAGGGCGAT |
| | | | CGGTGCGGGCCTCTTCGCTATTACGCCAGCTGGCG |
| | | | AAAGGGGGATGTGCTGCAAGGCGATTAAGTTGGGT |
| | | | AACGCCAGGGTTTTCCCAGTCACGACGTTGTAAAA |
| | | | CGACGGCCAGTGAATTGACGCGTATTGGGATGAGG |
| | | | TCCTGTATTAGAGGTCACGTGAGTGTTTTGCGA |
| | | | CATTTTGCGACACCATGTGGTCACGCTGGGTAT |
| | | | TTAAGCCCGAGTGAGCACGCAGGGTCTCCATTT |
| | | | TGAAGCGGGAGGTTTGAACGCGCAGCCGCCATGC |
| | | | CGGGGTTTTACGAGATTGTGATTAAGGTCCCCAGCGA |
| | | | CCTTGACGAGCATCTGCCCGGCATTTCTGACAGCTTTG |
| | | | TGAACTGGGTGGCCGAGAAGGAATGGGAGTTGCCGC |
| | | | CAGATTCTGACATGGATCTGAATCTGATTGAGCAGGCA |
| | | | CCCCTGACCGTGGCCGAGAAGCTGCAGCGCGACTTTC |
| | | | TGACGGAATGGCGCCGTGTGAGTAAGGCCCCGGAGG |
| | | | CTCTTTTCTTTGTGCAATTTGAGAAGGGAGAGAGCTAC |
| | | | TTCCACATGCACGTGCTCGTGGAAACCACCGGGGTGA |
| | | | AATCCATGGTTTTGGGACGTTTCCTGAGTCAGATTCGC |
| | | | GAAAAACTGATTCAGAGAATTTACCGCGGGATCGAGC |
| | | | CGACTTTGCCAAACTGGTTCGCGGTCACAAAGACCAG |
| | | | AAATGGCGCCGGAGGCGGGAACAAGGTGGTGGATGA |
| | | | GTGCTACATCCCCAATTACTTGCTCCCCAAAACCCAGC |
| | | | CTGAGCTCCAGTGGGCGTGGACTAATATGGAACAGTA |
| | | | TTTAAGCGCCTGTTTGAATCTCACGGAGCGTAAACGGT |
| | | | TGGTGGCGCAGCATCTGACGCACGTGTCGCAGACGCA |
| | | | GGAGCAGAACAAAGAGAATCAGAATCCCAATTCTGATG |
| | | | CGCCGGTGATCAGATCAAAAACTTCAGCCAGGTACAT |
| | | | GGAGCTGGTCGGGTGGCTCGTGGACAAGGGGATTAC |
| | | | CTCGGAGAAGCAGTGGATCCAGGAGGACCAGGCCTC |
| | | | ATACATCTCCTTCAATGCGGCCTCCAACTCGCGGTCCC |
| | | | AAATCAAGGCTGCCTTGGACAATGCGGGAAAGATTATG |
| | | | AGCCTGACTAAAACCGCCCCCGACTACCTGGTGGGCC |
| | | | AGCAGCCCGTGGAGGACATTTCCAGCAATCGGATTTAT |
| | | | AAAATTTTGGAACTAAACGGGTACGATCCCCAATATGC |
| | | | GGCTTCCGTCTTTCTGGGATGGGCCACGAAAAAGTTC |
| | | | GGCAAGAGGAACACCATCTGGCTGTTTGGGCCTGCAA |
| | | | CTACCGGGAAGACCAACATCGCGGAGGCCATAGCCCA |
| | | | CACTGTGCCCTTCTACGGGTGCGTAAACTGGACCAAT |
| | | | GAGAACTTTCCCTTCAACGACTGTGTCGACAAGATGGT |
| | | | GATCTGGTGGGAGGAGGGGAAGATGACCGCCAAGGT |
| | | | CGTGGAGTCGGCCAAAGCCATTCTCGGAGGAAGCAAG |
| | | | GTGCGCGTGGACCAGAAATGCAAGTCCTCGGCCCAGA |
| | | | TAGACCCGACTCCCGTGATCGTCACCTCCAACACCAA |
| | | | CATGTGCGCCGTGATTGACGGGAACTCAACGACCTTC |
| | | | GAACACCAGCAGCCGTTGCAAGACCGGATGTTCAAAT |
| | | | TTGAACTCACCCGCCGTCTGGATCATGACTTTGGGAAG |
| | | | GTCACCAAGCAGGAAGTCAAAGACTTTTTCCGGTGGG |
| | | | CAAAGGATCACGTGGTTGAGGTGGAGCATGAATTCTA |
| | | | CGTCAAAAAGGGTGGAGCCAAGAAAAGACCCGCCCCC |
| | | | AGTGACGCAGATATAAGTGAGCCCAAACGGGTGCGCG |
| | | | AGTCAGTTGCGCAGCCATCGACGTCAGACGCGGAAGC |
| | | | TTCGATCAACTACGCAGACAGGTACCAAAACAAATGTT |
| | | | CTCGTCACGTGGGCATGAATCTGATGCTGTTTCCCTGC |
| | | | AGACAATGCGAGAGAATGAATCAGAATTCAAATATCTG |
| | | | CTTCACTCACGGACAGAAAGACTGTTTAGAGTGCTTTC |
| | | | CCGTGTCAGAATCTCAACCCGTTTCTGTCGTCAAAAAG |
| | | | GCGTATCAGAAACTGTGCTACATTCATCATATCATGGG |
| | | | AAAGGTGCCAGACGCTTGCACTGCCTGCGATCTGGTC |
| | | | AATGTGGATTTGGATGACTGCATCTTTGAACAATAAAT |
| | | | GATTTAAATCAGGTatgtcttttgttgatcaccctccagattggttggaag |
| | | | aagttggtgaaggtcttcgcgagtttttgggccttgaagcgggcccaccgaaaccaa |
| | | | aacccaatcagcagcatcaagatcaagcccgtggtcttgtgctgcctggttataactat |
| | | | ctcggacccggaaacggtctcgatcgaggagagcctgtcaacagggcagacgag |
| | | | gtcgcgcgagagcacgacatctcgtacaacgagcagcttgaggcgggagacaac |
| | | | ccctacctcaagtacaaccacgcggacgccgagtttcaggagaagctcgccgacg |
| | | | acacatccttcgggggaaacctcggaaaggcagtctttcaggccaagaaaagggtt |
| | | | ctcgaaccttttggcctggttgaagagggtgctaagacggcccctaccggaaagcg |
| | | | gatagacgaccactttccaaaaagaaagaaggctcggaccgaagaggactccaag |
| | | | ccttccacctcgtcagacgccgaagctggacccagcggatcccagcagctgcaaat |
| | | | cccagcccaaccagcctcaagtttgggagctgatacaatgtctgcgggaggtggcg |
| | | | gcccattgggcgacaataaccaaggtgccgatggagtgggcaatgcctcgggaga |
| | | | ttggcattgcgattccacgtggatgggggacagagtcgtcaccaagtccacccgaa |
| | | | cctgggtgctgcccagctacaacaaccaccagtaccgagagatcaaaagcggctc |
| | | | cgtcgacggaagcaacgccaacgcctactttggatacagcaccccctgggggtact |
| | | | ttgactttaaccgcttccacagccactggagcccccgagactggcaaagactcatca |
| | | | acaactactggggcttcagaccccggtccctcagagtcaaaatcttcaacattcaagt |
| | | | caaagaggtcacggtgcaggactccaccaccaccatcgccaacaacctcacctcca |
| | | | ccgtccaagtgtttacggacgacgactaccagctgccctacgtcgtcggcaacggg |
| | | | accgagggatgcctgccggccttccctccgcaggtctttacgctgccgcagtacggt |
| | | | tacgcgacgctgaaccgcgacaacacagaaaatcccaccgagaggagcagcttct |
| | | | tctgcctagagtactttcccagcaagatgctgagaacgggcaacaactttgagtttac |
| | | | ctacaactttgaggaggtgcccttccactccagcttcgctcccagtcagaacctgttca |
| | | | agctggccaacccgctggtggaccagtacttgtaccgcttcgtgagcacaaataaca |
| | | | ctggcggagtccagttcaacaagaacctggccgggagatacgccaacacctacaa |
| | | | aaactggttcccggggcccatgggccgaacccagggctggaacctgggctccgg |
| | | | ggtcaaccgcgccagtgtcagcgccttcgccacgaccaataggatggagctcgag |
| | | | ggcgcgagttaccaggtgcccccgcagccgaacggcatgaccaacaacctccag |
| | | | ggcagcaacacctatgccctggagaacactatgatcttcaacagccagccggcgaa |
| | | | cccgggcaccaccgccacgtacctcgagggcaacatgctcatcaccagcgagagc |
| | | | gagacgcagccggtgaaccgcgtggcgtacaacgtcggcgggcagatcgccacc |
| | | | aacaaccagagctccaccactgcccccgcgaccggcacgtacaacctccaggaaa |
| | | | tcgtgcccggcagcgtgtggatggagagggacgtgtacctccaaggacccatctgg |
| | | | gccaagatcccagagacgggggcgcactttcacccctctccggccatgggcggatt |
| | | | cggactcaaacacccaccgcccatgatgctcatcaagaacacgcctgtgcccggaa |
| | | | atatcaccagcttctcggacgtgcccgtcagcagcttcatcacccagtacagcaccg |
| | | | ggcaggtcaccgtggagatggagtgggagctcaagaaggaaaactccaagaggt |
| | | | ggaacccagagatccagtacacaaacaactacaacgacccccagtttgtggactttg |
| | | | ccccggacagcaccggggaatacagaaccaccagacctatcggaacccgatacct |
| | | | tacccgacccctttaattgcttgttaatcaataaaccgtttaattcgtttcagttgaa |
| | | | ctttggtctctgcgaagggcgaattcgtttaaacctgcaggactagaggtcctgt |
| | | | attagaggtcacgtgagtgttttgcgacattttgcgacaccatgtggtcacgctg |
| | | | ggtatttaagcccgagtgagcacgcagggtctccattttgaagcgggaggtttg |
| | | | aacgcgcagccgccaagccgaattctgcagatccggttaattcgtttcagttgaacttt |
| | | | ggtctcctgtccttcttatcttatcggttaccatagaaactggttacttattaactgcttg |
| | | | gtgcgcttcgcgataaaagacttacgtcatcgggttacccctagtgatggagcggccgc |
| | | | tttcagttgaactttggtctctgcgtatttctttcttatctagtttccatgctctagaggt |
| | | | cctgtattagaggtcacgtgagtgttttgcgacattttgcgacaccatgtggtcacgct |
| | | | gggtatttaagcccgagtgagcacgcagggtctccattttgaaggggaggttt |
| | | | gaacgcgcagccgccaagccgaattctgcagaatcccaatGGCGCGCCG |
| | | | AGCTTGGCTCGAGCATGGTCATAGCTGTTTCCTGTG |
| | | | TGAAATTGTTATCCGCTCACAATTCCACACAACATA |
| | | | CGAGCCGGAAGCATAAAGTGTAAAGCCTGGGGTGC |
| | | | CTAATGAGTGAGCTAACTCACATTAATTGCGTTGC |
| | | | GCTCACTGCCCGCTTTCCAGTCGGGAAACCTGTCGT |
| | | | GCCAGCTGCATTAATGAATCGGCCAACGCGCGGGG |
| | | | AGAGGCGGTTTGCGTATTGGGCGCTCTTCCGCTTCC |
| | | | TCGCTCACTGACTCGCTGCGCTCGGTCGTTCGGCTG |
| | | | CGGCGAGCGGTATCAGCTCACTCAAAGGCGGTAAT |
| | | | ACGGTTATCCACAGAATCAGGGGATAACGCAGGAA |
| | | | AGAACATGTGAGCAAAAGGCCAGCAAAAGGCCAG |
| | | | GAACCGTAAAAAGGCCGCGTTGCTGGCGTTTTTCC |
| | | | ATAGGCTCCGCCCCCCTGACGAGCATCACAAAAAT |
| | | | CGACGCTCAAGTCAGAGGTGGCGAAACCCGACAG |
| | | | GACTATAAAGATACCAGGCGTTTCCCCCTGGAAGC |
| | | | TCCCTCGTGCGCTCTCCTGTTCCGACCCTGCCGCTT |
| | | | ACCGGATACCTGTCCGCCTTTCTCCCTTCGGGAAGC |
| | | | GTGGCGCTTTCTCATAGCTCACGCTGTAGGTATCTC |
| | | | AGTTCGGTGTAGGTCGTTCGCTCCAAGCTGGGCTGT |
| | | | GTGCACGAACCCCCCGTTCAGCCCGACCGCTGCGC |
| | | | CTTATCCGGTAACTATCGTCTTGAGTCCAACCCGGT |
| | | | AAGACACGACTTATCGCCACTGGCAGCAGCCACTG |
| | | | GTAACAGGATTAGCAGAGCGAGGTATGTAGGCGGT |
| | | | GCTACAGAGTTCTTGAAGTGGTGGCCTAACTACGG |
| | | | CTACACTAGAAGAACAGTATTTGGTATCTGCGCTCT |
| | | | GCTGAAGCCAGTTACCTTCGGAAAAAGAGTTGGTA |
| | | | GCTCTTGATCCGGCAAACAAACCACCGCTGGTAGC |
| | | | GGTGGTTTTTTTGTTTGCAAGCAGCAGATTACGCGC |
| | | | AGAAAAAAAGGATCTCAAGAAGATCCTTTGATCTT |
| | | | TTCTACGGGGTCTGACGCTCAGTGGAACGAAAACT |
| | | | CACGTTAAGGGATTTTGGTCATGAGATTATCAAAA |
| | | | AGGATCTTCACCTAGATCCTTTTAAATTAAAAATGA |
| | | | AGTTTTAAATCAATCTAAAGTATATATGAGTAAAC |
| | | | TTGGTCTGACAGTTAGAAAAACTCATCGAGCATCA |
| | | | AATGAAACTGCAATTTATTCATATCAGGATTATCA |
| | | | ATACCATATTTTTGAAAAAGCCGTTTCTGTAATGAA |
| | | | GGAGAAAACTCACCGAGGCAGTTCCATAGGATGGC |
| | | | AAGATCCTGGTATCGGTCTGCGATTCCGACTCGTCC |
| | | | AACATCAATACAACCTATTAATTTCCCCTCGTCAAA |
| | | | AATAAGGTTATCAAGTGAGAAATCACCATGAGTGA |
| | | | CGACTGAATCCGGTGAGAATGGCAAAAGTTTATGC |
| | | | ATTTCTTTCCAGACTTGTTCAACAGGCCAGCCATTA |
| | | | CGCTCGTCATCAAAATCACTCGCATCAACCAAACC |
| | | | GTTATTCATTCGTGATTGCGCCTGAGCGAGACGAA |
| | | | ATACGCGATCGCTGTTAAAAGGACAATTACAAACA |
| | | | GGAATCGAATGCAACCGGCGCAGGAACACTGCCA |
| | | | GCGCATCAACAATATTTTCACCTGAATCAGGATATT |
| | | | CTTCTAATACCTGGAATGCTGTTTTCCCAGGGATCG |
| | | | CAGTGGTGAGTAACCATGCATCATCAGGAGTACGG |
| | | | ATAAAATGCTTGATGGTCGGAAGAGGCATAAATTC |
| | | | CGTCAGCCAGTTTAGTCTGACCATCTCATCTGTAAC |
| | | | ATCATTGGCAACGCTACCTTTGCCATGTTTCAGAAA |
| | | | CAACTCTGGCGCATCGGGCTTCCCATACAATCGAT |
| | | | AGATTGTCGCACCTGATTGCCCGACATTATCGCGA |
| | | | GCCCATTTATACCCATATAAATCAGCATCCATGTTG |
| | | | GAATTTAATCGCGGCCTAGAGCAAGACGTTTCCCG |
| | | | TTGAATATGGCTCATACTCTTCCTTTTTCAATATTAT |
| | | | TGAAGCATTTATCAGGGTTATTGTCTCATGAGCGG |
| | | | ATACATATTTGAATGTATTTAGAAAAATAAACAAA |
| | | | TAGGGGTTCCGCGCACATTTCCCCGAAAAGTGCCA |
| | | | CCTGACGTCTAAGAAACCATTATTATCATGACATTA |
| | | | ACCTATAAAAATAGGCGTATCACGAGGCCCTTTCG |
| | | | TC |
| 53 | pYZZ448 | p5- | TCGCGCGTTTCGGTGATGACGGTGAAAACCTCTGACACAT |
| | | AltRep2Cap5- | GCAGCTCCCGGAGACGGTCACAGCTTGTCTGTAAGCGGAT |
| | | p5.p5 | GCCGGGAGCAGACAAGCCCGTCAGGGCGCGTCAGCGGGTG |
| | | | TTGGCGGGTGTCGGGGCTGGCTTAACTATGCGGCATCAGA |
| | | | GCAGATTGTACTGAGAGTGCACCATATGCGGTGTGAAATA |
| | | | CCGCACAGATGCGTAAGGAGAAAATACCGCATCAGGCGCC |
| | | | ATTCGCCATTCAGGCTGCGCAACTGTTGGGAAGGGCGATC |
| | | | GGTGCGGGCCTCTTCGCTATTACGCCAGCTGGCGAAAGGG |
| | | | GGATGTGCTGCAAGGCGATTAAGTTGGGTAACGCCAGGGT |
| | | | TTTCCCAGTCACGACGTTGTAAAACGACGGCCAGTGAATT |
| | | | GACGCGTATTGGGATGAGGTCCTGTATTAGAGGTCACGT |
| | | | GAGTGTTTTGCGACATTTTGCGACACCATGTGGTCACG |
| | | | CTGGGTATTTAAGCCCGAGTGAGCACGCAGGGTCTCCA |
| | | | TTTTGAAGCGGGAGGTTTGAACGCGCAGCCGCCACGCCG |
| | | | GGGTTTTACGAGATTGTGATTAAGGTCCCCAGCGACCTTGAC |
| | | | GAGCATCTGCCCGGCATTTCTGACAGCTTTGTGAACTGGGTG |
| | | | GCCGAGAAGGAATGGGAGTTGCCGCCAGATTCTGACATGGAT |
| | | | CTGAATCTGATTGAGCAGGCACCCCTGACCGTGGCCGAGAAG |
| | | | CTGCAGCGCGACTTTCTGACGGAATGGCGCCGTGTGAGTAAG |
| | | | GCCCCGGAGGCTCTTTTCTTTGTGCAATTTGAGAAGGGAGAG |
| | | | AGCTACTTCCACATGCACGTGCTCGTGGAAACCACCGGGGTG |
| | | | AAATCCATGGTTTTGGGACGTTTCCTGAGTCAGATTCGCGAA |
| | | | AAACTGATTCAGAGAATTTACCGCGGGATCGAGCCGACTTTG |
| | | | CCAAACTGGTTCGCGGTCACAAAGACCAGAAATGGCGCCGGA |
| | | | GGCGGGAACAAGGTGGTGGATGAGTGCTACATCCCCAATTAC |
| | | | TTGCTCCCCAAAACCCAGCCTGAGCTCCAGTGGGCGTGGACT |
| | | | AATATGGAACAGTATTTAAGCGCCTGTTTGAATCTCACGGAG |
| | | | CGTAAACGGTTGGTGGCGCAGCATCTGACGCACGTGTCGCAG |
| | | | ACGCAGGAGCAGAACAAAGAGAATCAGAATCCCAATTCTGAT |
| | | | GCGCCGGTGATCAGATCAAAAACTTCAGCCAGGTACATGGAG |
| | | | CTGGTCGGGTGGCTCGTGGACAAGGGGATTACCTCGGAGAAG |
| | | | CAGTGGATCCAGGAGGACCAGGCCTCATACATCTCCTTCAAT |
| | | | GCGGCCTCCAACTCGCGGTCCCAAATCAAGGCTGCCTTGGACA |
| | | | ATGCGGGAAAGATTATGAGCCTGACTAAAACCGCCCCCGACT |
| | | | ACCTGGTGGGCCAGCAGCCCGTGGAGGACATTTCCAGCAATC |
| | | | GGATTTATAAAATTTTGGAACTAAACGGGTACGATCCCCAAT |
| | | | ATGCGGCTTCCGTCTTTCTGGGATGGGCCACGAAAAAGTTCG |
| | | | GCAAGAGGAACACCATCTGGCTGTTTGGGCCTGCAACTACCG |
| | | | GGAAGACCAACATCGCGGAGGCCATAGCCCACACTGTGCCCT |
| | | | TCTACGGGTGCGTAAACTGGACCAATGAGAACTTTCCCTTCA |
| | | | ACGACTGTGTCGACAAGATGGTGATCTGGTGGGAGGAGGGGA |
| | | | AGATGACCGCCAAGGTCGTGGAGTCGGCCAAAGCCATTCTCG |
| | | | GAGGAAGCAAGGTGCGCGTGGACCAGAAATGCAAGTCCTCGG |
| | | | CCCAGATAGACCCGACTCCCGTGATCGTCACCTCCAACACCAA |
| | | | CATGTGCGCCGTGATTGACGGGAACTCAACGACCTTCGAACA |
| | | | CCAGCAGCCGTTGCAAGACCGGATGTTCAAATTTGAACTCAC |
| | | | CCGCCGTCTGGATCATGACTTTGGGAAGGTCACCAAGCAGGA |
| | | | AGTCAAAGACTTTTTCCGGTGGGCAAAGGATCACGTGGTTGA |
| | | | GGTGGAGCATGAATTCTACGTCAAAAAGGGTGGAGCCAAGAA |
| | | | AAGACCCGCCCCCAGTGACGCAGATATAAGTGAGCCCAAACG |
| | | | GGTGCGCGAGTCAGTTGCGCAGCCATCGACGTCAGACGCGGA |
| | | | AGCTTCGATCAACTACGCAGACAGGTACCAAAACAAATGTTC |
| | | | TCGTCACGTGGGCATGAATCTGATGCTGTTTCCCTGCAGACA |
| | | | ATGCGAGAGAATGAATCAGAATTCAAATATCTGCTTCACTCA |
| | | | CGGACAGAAAGACTGTTTAGAGTGCTTTCCCGTGTCAGAATC |
| | | | TCAACCCGTTTCTGTCGTCAAAAAGGCGTATCAGAAACTGTG |
| | | | CTACATTCATCATATCATGGGAAAGGTGCCAGACGCTTGCAC |
| | | | TGCCTGCGATCTGGTCAATGTGGATTTGGATGACTGCATCTT |
| | | | TGAACAATAAATGATTTAAATCAGGTatgtcttttgttgatcaccct |
| | | | ccagattggttggaagaagttggtgaaggtcttcgcgagtttttgggccttgaag |
| | | | cgggcccaccgaaaccaaaacccaatcagcagcatcaagatcaagcccgtggt |
| | | | cttgtgctgcctggttataactatctcggacccggaaacggtctcgatcgaggag |
| | | | agcctgtcaacagggcagacgaggtcgcgcgagagcacgacatctcgtacaac |
| | | | gagcagcttgaggcgggagacaacccctacctcaagtacaaccacgcggacgc |
| | | | cgagtttcaggagaagctcgccgacgacacatccttcgggggaaacctcggaa |
| | | | aggcagtctttcaggccaagaaaagggttctcgaaccttttggcctggttgaaga |
| | | | gggtgctaagacggcccctaccggaaagcggatagacgaccactttccaaaaa |
| | | | gaaagaaggctcggaccgaagaggactccaagccttccacctcgtcagacgcc |
| | | | gaagctggacccagcggatcccagcagctgcaaatcccagcccaaccagcctc |
| | | | aagtttgggagctgatacaatgtctgcgggaggtggcggcccattgggcgacaa |
| | | | taaccaaggtgccgatggagtgggcaatgcctcgggagattggcattgcgattc |
| | | | cacgtggatgggggacagagtcgtcaccaagtccacccgaacctgggtgctgc |
| | | | ccagctacaacaaccaccagtaccgagagatcaaaagcggctccgtcgacgga |
| | | | agcaacgccaacgcctactttggatacagcaccccctgggggtactttgacttta |
| | | | accgcttccacagccactggagcccccgagactggcaaagactcatcaacaac |
| | | | tactggggcttcagaccccggtccctcagagtcaaaatcttcaacattcaagtca |
| | | | aagaggtcacggtgcaggactccaccaccaccatcgccaacaacctcacctcc |
| | | | accgtccaagtgtttacggacgacgactaccagctgccctacgtcgtcggcaac |
| | | | gggaccgagggatgcctgccggccttccctccgcaggtctttacgctgccgcagt |
| | | | acggttacgcgacgctgaaccgcgacaacacagaaaatcccaccgagaggag |
| | | | cagcttcttctgcctagagtactttcccagcaagatgctgagaacgggcaacaac |
| | | | tttgagtttacctacaactttgaggaggtgcccttccactccagcttcgctcccagt |
| | | | cagaacctgttcaagctggccaacccgctggtggaccagtacttgtaccgcttcg |
| | | | tgagcacaaataacactggcggagtccagttcaacaagaacctggccgggaga |
| | | | tacgccaacacctacaaaaactggttcccggggcccatgggccgaacccaggg |
| | | | ctggaacctgggctccggggtcaaccgcgccagtgtcagcgccttcgccacgac |
| | | | caataggatggagctcgagggcgcgagttaccaggtgcccccgcagccgaacg |
| | | | gcatgaccaacaacctccagggcagcaacacctatgccctggagaacactatg |
| | | | atcttcaacagccagccggcgaacccgggcaccaccgccacgtacctcgaggg |
| | | | caacatgctcatcaccagcgagagcgagacgcagccggtgaaccgcgtggcgt |
| | | | acaacgtcggcgggcagatggccaccaacaaccagagctccaccactgccccc |
| | | | gcgaccggcacgtacaacctccaggaaatcgtgcccggcagcgtgtggatgga |
| | | | gagggacgtgtacctccaaggacccatctgggccaagatcccagagacggggg |
| | | | cgcactttcacccctctccggccatgggcggattcggactcaaacacccaccgc |
| | | | ccatgatgctcatcaagaacacgcctgtgcccggaaatatcaccagcttctcgg |
| | | | acgtgcccgtcagcagcttcatcacccagtacagcaccgggcaggtcaccgtgg |
| | | | agatggagtgggagctcaagaaggaaaactccaagaggtggaacccagagat |
| | | | ccagtacacaaacaactacaacgacccccagtttgtggactttgccccggacag |
| | | | caccggggaatacagaaccaccagacctatcggaacccgataccttacccgac |
| | | | ccctttaattgcttcttaatcaataaaccgtttaattcgtttcagttgaactttg |
| | | | gtctctgcgaagggcgaattcgtttaaacctgcaggactagaggtcctgtatta |
| | | | gaggtcacgtgagtgttttgcgacattttgcgacaccatgtggtcacgctgg |
| | | | gtatttaagcccgagtgagcacgcagggtctccattttgaagcgggaggtt |
| | | | tgaacgcgcagccgccaagccgaattctgcagatccggttaattcgtttcagttg |
| | | | aactttggtctcctgtccttcttatcttatcggttaccatagaaactggttacttatt |
| | | | aactgcttggtgcgcttcgcgataaaagacttacgtcatcgggttacccctagtg |
| | | | atggagcggccgctttcagttgaactttggtctctgcgtatttctttcttatctagtt |
| | | | tccatgctctagaggtcctgtattagaggtcacgtgagtgttttgcgacatttt |
| | | | gcgacaccatgtggtcacgctgggtatttaagcccgagtgagcacgcagg |
| | | | gtctccattttgaagcgggaggtttgaacgcgcagccgccaagccgaattctg |
| | | | cagaatcccaatGGCGCGCCGAGCTTGGCTCGAGCATGGTCAT |
| | | | AGCTGTTTCCTGTGTGAAATTGTTATCCGCTCACAATTCC |
| | | | ACACAACATACGAGCCGGAAGCATAAAGTGTAAAGCCTGG |
| | | | GGTGCCTAATGAGTGAGCTAACTCACATTAATTGCGTTGC |
| | | | GCTCACTGCCCGCTTTCCAGTCGGGAAACCTGTCGTGCCAG |
| | | | CTGCATTAATGAATCGGCCAACGCGCGGGGAGAGGCGGTT |
| | | | TGCGTATTGGGCGCTCTTCCGCTTCCTCGCTCACTGACTCG |
| | | | CTGCGCTCGGTCGTTCGGCTGCGGCGAGCGGTATCAGCTCA |
| | | | CTCAAAGGCGGTAATACGGTTATCCACAGAATCAGGGGAT |
| | | | AACGCAGGAAAGAACATGTGAGCAAAAGGCCAGCAAAAGG |
| | | | CCAGGAACCGTAAAAAGGCCGCGTTGCTGGCGTTTTTCCA |
| | | | TAGGCTCCGCCCCCCTGACGAGCATCACAAAAATCGACGCT |
| | | | CAAGTCAGAGGTGGCGAAACCCGACAGGACTATAAAGATA |
| | | | CCAGGCGTTTCCCCCTGGAAGCTCCCTCGTGCGCTCTCCTG |
| | | | TTCCGACCCTGCCGCTTACCGGATACCTGTCCGCCTTTCTC |
| | | | CCTTCGGGAAGCGTGGCGCTTTCTCATAGCTCACGCTGTAG |
| | | | GTATCTCAGTTCGGTGTAGGTCGTTCGCTCCAAGCTGGGC |
| | | | TGTGTGCACGAACCCCCCGTTCAGCCCGACCGCTGCGCCTT |
| | | | ATCCGGTAACTATCGTCTTGAGTCCAACCCGGTAAGACAC |
| | | | GACTTATCGCCACTGGCAGCAGCCACTGGTAACAGGATTA |
| | | | GCAGAGCGAGGTATGTAGGCGGTGCTACAGAGTTCTTGAA |
| | | | GTGGTGGCCTAACTACGGCTACACTAGAAGAACAGTATTT |
| | | | GGTATCTGCGCTCTGCTGAAGCCAGTTACCTTCGGAAAAA |
| | | | GAGTTGGTAGCTCTTGATCCGGCAAACAAACCACCGCTGG |
| | | | TAGCGGTGGTTTTTTTGTTTGCAAGCAGCAGATTACGCGC |
| | | | AGAAAAAAAGGATCTCAAGAAGATCCTTTGATCTTTTCTA |
| | | | CGGGGTCTGACGCTCAGTGGAACGAAAACTCACGTTAAGG |
| | | | GATTTTGGTCATGAGATTATCAAAAAGGATCTTCACCTAG |
| | | | ATCCTTTTAAATTAAAAATGAAGTTTTAAATCAATCTAAA |
| | | | GTATATATGAGTAAACTTGGTCTGACAGTTAGAAAAACTC |
| | | | ATCGAGCATCAAATGAAACTGCAATTTATTCATATCAGGA |
| | | | TTATCAATACCATATTTTTGAAAAAGCCGTTTCTGTAATG |
| | | | AAGGAGAAAACTCACCGAGGCAGTTCCATAGGATGGCAAG |
| | | | ATCCTGGTATCGGTCTGCGATTCCGACTCGTCCAACATCAA |
| | | | TACAACCTATTAATTTCCCCTCGTCAAAAATAAGGTTATC |
| | | | AAGTGAGAAATCACCATGAGTGACGACTGAATCCGGTGAG |
| | | | AATGGCAAAAGTTTATGCATTTCTTTCCAGACTTGTTCAA |
| | | | CAGGCCAGCCATTACGCTCGTCATCAAAATCACTCGCATCA |
| | | | ACCAAACCGTTATTCATTCGTGATTGCGCCTGAGCGAGAC |
| | | | GAAATACGCGATCGCTGTTAAAAGGACAATTACAAACAGG |
| | | | AATCGAATGCAACCGGCGCAGGAACACTGCCAGCGCATCA |
| | | | ACAATATTTTCACCTGAATCAGGATATTCTTCTAATACCT |
| | | | GGAATGCTGTTTTCCCAGGGATCGCAGTGGTGAGTAACCA |
| | | | TGCATCATCAGGAGTACGGATAAAATGCTTGATGGTCGGA |
| | | | AGAGGCATAAATTCCGTCAGCCAGTTTAGTCTGACCATCT |
| | | | CATCTGTAACATCATTGGCAACGCTACCTTTGCCATGTTTC |
| | | | AGAAACAACTCTGGCGCATCGGGCTTCCCATACAATCGAT |
| | | | AGATTGTCGCACCTGATTGCCCGACATTATCGCGAGCCCAT |
| | | | TTATACCCATATAAATCAGCATCCATGTTGGAATTTAATC |
| | | | GCGGCCTAGAGCAAGACGTTTCCCGTTGAATATGGCTCAT |
| | | | ACTCTTCCTTTTTCAATATTATTGAAGCATTTATCAGGGT |
| | | | TATTGTCTCATGAGCGGATACATATTTGAATGTATTTAGA |
| | | | AAAATAAACAAATAGGGGTTCCGCGCACATTTCCCCGAAA |
| | | | AGTGCCACCTGACGTCTAAGAAACCATTATTATCATGACA |
| | | | TTAACCTATAAAAATAGGCGTATCACGAGGCCCTTTCGTC |
| 54 | pYZZ436 | Rep2Cap5-p5. | TCGCGCGTTTCGGTGATGACGGTGAAAACCTCTGACACAT |
| | | p5 | GCAGCTCCCGGAGACGGTCACAGCTTGTCTGTAAGCGGAT |
| | | | GCCGGGAGCAGACAAGCCCGTCAGGGCGCGTCAGCGGGTG |
| | | | TTGGCGGGTGTCGGGGCTGGCTTAACTATGCGGCATCAGA |
| | | | GCAGATTGTACTGAGAGTGCACCATATGCGGTGTGAAATA |
| | | | CCGCACAGATGCGTAAGGAGAAAATACCGCATCAGGCGCC |
| | | | ATTCGCCATTCAGGCTGCGCAACTGTTGGGAAGGGCGATC |
| | | | GGTGCGGGCCTCTTCGCTATTACGCCAGCTGGCGAAAGGG |
| | | | GGATGTGCTGCAAGGCGATTAAGTTGGGTAACGCCAGGGT |
| | | | TTTCCCAGTCACGACGTTGTAAAACGACGGCCAGTGAATT |
| | | | GACGCGTATTGGGATCGCGCAGCCGCCATGCCGGGGTTTTA |
| | | | CGAGATTGTGATTAAGGTCCCCAGCGACCTTGACGAGCATCT |
| | | | GCCCGGCATTTCTGACAGCTTTGTGAACTGGGTGGCCGAGAA |
| | | | GGAATGGGAGTTGCCGCCAGATTCTGACATGGATCTGAATCT |
| | | | GATTGAGCAGGCACCCCTGACCGTGGCCGAGAAGCTGCAGCG |
| | | | CGACTTTCTGACGGAATGGCGCCGTGTGAGTAAGGCCCCGGA |
| | | | GGCTCTTTTCTTTGTGCAATTTGAGAAGGGAGAGAGCTACTT |
| | | | CCACATGCACGTGCTCGTGGAAACCACCGGGGTGAAATCCAT |
| | | | GGTTTTGGGACGTTTCCTGAGTCAGATTCGCGAAAAACTGAT |
| | | | TCAGAGAATTTACCGCGGGATCGAGCCGACTTTGCCAAACTG |
| | | | GTTCGCGGTCACAAAGACCAGAAATGGCGCCGGAGGCGGGAA |
| | | | CAAGGTGGTGGATGAGTGCTACATCCCCAATTACTTGCTCCC |
| | | | CAAAACCCAGCCTGAGCTCCAGTGGGCGTGGACTAATATGGA |
| | | | ACAGTATTTAAGCGCCTGTTTGAATCTCACGGAGCGTAAACG |
| | | | GTTGGTGGCGCAGCATCTGACGCACGTGTCGCAGACGCAGGA |
| | | | GCAGAACAAAGAGAATCAGAATCCCAATTCTGATGCGCCGGT |
| | | | GATCAGATCAAAAACTTCAGCCAGGTACATGGAGCTGGTCGG |
| | | | GTGGCTCGTGGACAAGGGGATTACCTCGGAGAAGCAGTGGAT |
| | | | CCAGGAGGACCAGGCCTCATACATCTCCTTCAATGCGGCCTCC |
| | | | AACTCGCGGTCCCAAATCAAGGCTGCCTTGGACAATGCGGGA |
| | | | AAGATTATGAGCCTGACTAAAACCGCCCCCGACTACCTGGTG |
| | | | GGCCAGCAGCCCGTGGAGGACATTTCCAGCAATCGGATTTAT |
| | | | AAAATTTTGGAACTAAACGGGTACGATCCCCAATATGCGGCT |
| | | | TCCGTCTTTCTGGGATGGGCCACGAAAAAGTTCGGCAAGAGG |
| | | | AACACCATCTGGCTGTTTGGGCCTGCAACTACCGGGAAGACC |
| | | | AACATCGCGGAGGCCATAGCCCACACTGTGCCCTTCTACGGG |
| | | | TGCGTAAACTGGACCAATGAGAACTTTCCCTTCAACGACTGT |
| | | | GTCGACAAGATGGTGATCTGGTGGGAGGAGGGGAAGATGACC |
| | | | GCCAAGGTCGTGGAGTCGGCCAAAGCCATTCTCGGAGGAAGC |
| | | | AAGGTGCGCGTGGACCAGAAATGCAAGTCCTCGGCCCAGATA |
| | | | GACCCGACTCCCGTGATCGTCACCTCCAACACCAACATGTGCG |
| | | | CCGTGATTGACGGGAACTCAACGACCTTCGAACACCAGCAGC |
| | | | CGTTGCAAGACCGGATGTTCAAATTTGAACTCACCCGCCGTC |
| | | | TGGATCATGACTTTGGGAAGGTCACCAAGCAGGAAGTCAAAG |
| | | | ACTTTTTCCGGTGGGCAAAGGATCACGTGGTTGAGGTGGAGC |
| | | | ATGAATTCTACGTCAAAAAGGGTGGAGCCAAGAAAAGACCCG |
| | | | CCCCCAGTGACGCAGATATAAGTGAGCCCAAACGGGTGCGCG |
| | | | AGTCAGTTGCGCAGCCATCGACGTCAGACGCGGAAGCTTCGA |
| | | | TCAACTACGCAGACAGGTACCAAAACAAATGTTCTCGTCACG |
| | | | TGGGCATGAATCTGATGCTGTTTCCCTGCAGACAATGCGAGA |
| | | | GAATGAATCAGAATTCAAATATCTGCTTCACTCACGGACAGA |
| | | | AAGACTGTTTAGAGTGCTTTCCCGTGTCAGAATCTCAACCCG |
| | | | TTTCTGTCGTCAAAAAGGCGTATCAGAAACTGTGCTACATTC |
| | | | ATCATATCATGGGAAAGGTGCCAGACGCTTGCACTGCCTGCG |
| | | | ATCTGGTCAATGTGGATTTGGATGACTGCATCTTTGAACAAT |
| | | | AAATGATTTAAATCAGGTatgtcttttgttgatcaccctccagattggtt |
| | | | ggaagaagttggtgaaggtcttcgcgagtttttgggccttgaagcgggcccacc |
| | | | gaaaccaaaacccaatcagcagcatcaagatcaagcccgtggtcttgtgctgcc |
| | | | tggttataactatctcggacccggaaacggtctcgatcgaggagagcctgtcaa |
| | | | cagggcagacgaggtcgcgcgagagcacgacatctcgtacaacgagcagcttg |
| | | | aggcgggagacaacccctacctcaagtacaaccacgcggacgccgagtttcag |
| | | | gagaagctcgccgacgacacatccttcgggggaaacctcggaaaggcagtctt |
| | | | tcaggccaagaaaagggttctcgaaccttttggcctggttgaagagggtgctaa |
| | | | gacggcccctaccggaaagcggatagacgaccactttccaaaaagaaagaag |
| | | | gctcggaccgaagaggactccaagccttccacctcgtcagacgccgaagctgg |
| | | | acccagcggatcccagcagctgcaaatcccagcccaaccagcctcaagtttgg |
| | | | gagctgatacaatgtctgcgggaggtggcggcccattgggcgacaataaccaa |
| | | | ggtgccgatggagtgggcaatgcctcgggagattggcattgcgattccacgtgg |
| | | | atgggggacagagtcgtcaccaagtccacccgaacctgggtgctgcccagcta |
| | | | caacaaccaccagtaccgagagatcaaaagcggctccgtcgacggaagcaac |
| | | | gccaacgcctactttggatacagcaccccctgggggtactttgactttaaccgctt |
| | | | ccacagccactggagcccccgagactggcaaagactcatcaacaactactggg |
| | | | gcttcagaccccggtccctcagagtcaaaatcttcaacattcaagtcaaagaggt |
| | | | cacggtgcaggactccaccaccaccatcgccaacaacctcacctccaccgtcca |
| | | | agtgtttacggacgacgactaccagctgccctacgtcgtcggcaacgggaccga |
| | | | gggatgcctgccggccttccctccgcaggtctttacgctgccgcagtacggttac |
| | | | gcgacgctgaaccgcgacaacacagaaaatcccaccgagaggagcagcttctt |
| | | | ctgcctagagtactttcccagcaagatgctgagaacgggcaacaactttgagttt |
| | | | acctacaactttgaggaggtgcccttccactccagcttcgctcccagtcagaacc |
| | | | tgttcaagctggccaacccgctggtggaccagtacttgtaccgcttcgtgagcac |
| | | | aaataacactggcggagtccagttcaacaagaacctggccgggagatacgcca |
| | | | acacctacaaaaactggttcccggggcccatgggccgaacccagggctggaac |
| | | | ctgggctccggggtcaaccgcgccagtgtcagcgccttcgccacgaccaatagg |
| | | | atggagctcgagggcgcgagttaccaggtgcccccgcagccgaacggcatgac |
| | | | caacaacctccagggcagcaacacctatgccctggagaacactatgatcttcaa |
| | | | cagccagccggcgaacccgggcaccaccgccacgtacctcgagggcaacatg |
| | | | ctcatcaccagcgagagcgagacgcagccggtgaaccgcgtggcgtacaacgt |
| | | | cggcgggcagatggccaccaacaaccagagctccaccactgcccccgcgaccg |
| | | | gcacgtacaacctccaggaaatcgtgcccggcagcgtgtggatggagagggac |
| | | | gtgtacctccaaggacccatctgggccaagatcccagagacgggggcgcacttt |
| | | | cacccctctccggccatgggcggattcggactcaaacacccaccgcccatgatg |
| | | | ctcatcaagaacacgcctgtgcccggaaatatcaccagcttctcggacgtgccc |
| | | | gtcagcagcttcatcacccagtacagcaccgggcaggtcaccgtggagatgga |
| | | | gtgggagctcaagaaggaaaactccaagaggtggaacccagagatccagtac |
| | | | acaaacaactacaacgacccccagtttgtggactttgccccggacagcaccggg |
| | | | gaatacagaaccaccagacctatcggaacccgataccttacccgacccctttaa |
| | | | ttgcttgttaatcaataaaccgtttaattcgtttcagttgaactttggtctctgc |
| | | | gaagggcgaattcgtttaaacctgcaggactagaggtcctgtattagaggtca |
| | | | cgtgagtgttttgcgacattttgcgacaccatgtggtcacgctgggtattta |
| | | | agcccgagtgagcacgcagggtctccattttgaagcgggaggtttgaacg |
| | | | cgcagccgccaagccgaattctgcagatccggttaattcgtttcagttgaactttg |
| | | | gtctcctgtccttcttatcttatcggttaccatagaaactggttacttattaactgct |
| | | | tggtgcgcttcgcgataaaagacttacgtcatcgggttacccctagtgatggagc |
| | | | ggccgctttcagttgaactttggtctctgcgtatttctttcttatctagtttccatgct |
| | | | ctagaggtcctgtattagaggtcacgtgagtgttttgcgacattttgcgaca |
| | | | ccatgtggtcacgctgggtatttaagcccgagtgagcacgcagggtctcca |
| | | | ttttgaagcgggaggtttgaacgcgcagccgccaagccgaattctgcagaatc |
| | | | ccaatGGCGCGCCGAGCTTGGCTCGAGCATGGTCATAGCTGT |
| | | | TTCCTGTGTGAAATTGTTATCCGCTCACAATTCCACACAAC |
| | | | ATACGAGCCGGAAGCATAAAGTGTAAAGCCTGGGGTGCCT |
| | | | AATGAGTGAGCTAACTCACATTAATTGCGTTGCGCTCACT |
| | | | GCCCGCTTTCCAGTCGGGAAACCTGTCGTGCCAGCTGCATT |
| | | | AATGAATCGGCCAACGCGCGGGGAGAGGCGGTTTGCGTAT |
| | | | TGGGCGCTCTTCCGCTTCCTCGCTCACTGACTCGCTGCGCT |
| | | | CGGTCGTTCGGCTGCGGCGAGCGGTATCAGCTCACTCAAA |
| | | | GGCGGTAATACGGTTATCCACAGAATCAGGGGATAACGCA |
| | | | GGAAAGAACATGTGAGCAAAAGGCCAGCAAAAGGCCAGGA |
| | | | ACCGTAAAAAGGCCGCGTTGCTGGCGTTTTTCCATAGGCT |
| | | | CCGCCCCCCTGACGAGCATCACAAAAATCGACGCTCAAGTC |
| | | | AGAGGTGGCGAAACCCGACAGGACTATAAAGATACCAGGC |
| | | | GTTTCCCCCTGGAAGCTCCCTCGTGCGCTCTCCTGTTCCGA |
| | | | CCCTGCCGCTTACCGGATACCTGTCCGCCTTTCTCCCTTCG |
| | | | GGAAGCGTGGCGCTTTCTCATAGCTCACGCTGTAGGTATC |
| | | | TCAGTTCGGTGTAGGTCGTTCGCTCCAAGCTGGGCTGTGT |
| | | | GCACGAACCCCCCGTTCAGCCCGACCGCTGCGCCTTATCCG |
| | | | GTAACTATCGTCTTGAGTCCAACCCGGTAAGACACGACTT |
| | | | ATCGCCACTGGCAGCAGCCACTGGTAACAGGATTAGCAGA |
| | | | GCGAGGTATGTAGGCGGTGCTACAGAGTTCTTGAAGTGGT |
| | | | GGCCTAACTACGGCTACACTAGAAGAACAGTATTTGGTAT |
| | | | CTGCGCTCTGCTGAAGCCAGTTACCTTCGGAAAAAGAGTT |
| | | | GGTAGCTCTTGATCCGGCAAACAAACCACCGCTGGTAGCG |
| | | | GTGGTTTTTTTGTTTGCAAGCAGCAGATTACGCGCAGAAA |
| | | | AAAAGGATCTCAAGAAGATCCTTTGATCTTTTCTACGGGG |
| | | | TCTGACGCTCAGTGGAACGAAAACTCACGTTAAGGGATTT |
| | | | TGGTCATGAGATTATCAAAAAGGATCTTCACCTAGATCCT |
| | | | TTTAAATTAAAAATGAAGTTTTAAATCAATCTAAAGTATA |
| | | | TATGAGTAAACTTGGTCTGACAGTTAGAAAAACTCATCGA |
| | | | GCATCAAATGAAACTGCAATTTATTCATATCAGGATTATC |
| | | | AATACCATATTTTTGAAAAAGCCGTTTCTGTAATGAAGGA |
| | | | GAAAACTCACCGAGGCAGTTCCATAGGATGGCAAGATCCT |
| | | | GGTATCGGTCTGCGATTCCGACTCGTCCAACATCAATACA |
| | | | ACCTATTAATTTCCCCTCGTCAAAAATAAGGTTATCAAGT |
| | | | GAGAAATCACCATGAGTGACGACTGAATCCGGTGAGAATG |
| | | | GCAAAAGTTTATGCATTTCTTTCCAGACTTGTTCAACAGG |
| | | | CCAGCCATTACGCTCGTCATCAAAATCACTCGCATCAACCA |
| | | | AACCGTTATTCATTCGTGATTGCGCCTGAGCGAGACGAAA |
| | | | TACGCGATCGCTGTTAAAAGGACAATTACAAACAGGAATC |
| | | | GAATGCAACCGGCGCAGGAACACTGCCAGCGCATCAACAA |
| | | | TATTTTCACCTGAATCAGGATATTCTTCTAATACCTGGAA |
| | | | TGCTGTTTTCCCAGGGATCGCAGTGGTGAGTAACCATGCA |
| | | | TCATCAGGAGTACGGATAAAATGCTTGATGGTCGGAAGAG |
| | | | GCATAAATTCCGTCAGCCAGTTTAGTCTGACCATCTCATCT |
| | | | GTAACATCATTGGCAACGCTACCTTTGCCATGTTTCAGAA |
| | | | ACAACTCTGGCGCATCGGGCTTCCCATACAATCGATAGAT |
| | | | TGTCGCACCTGATTGCCCGACATTATCGCGAGCCCATTTAT |
| | | | ACCCATATAAATCAGCATCCATGTTGGAATTTAATCGCGG |
| | | | CCTAGAGCAAGACGTTTCCCGTTGAATATGGCTCATACTC |
| | | | TTCCTTTTTCAATATTATTGAAGCATTTATCAGGGTTATT |
| | | | GTCTCATGAGCGGATACATATTTGAATGTATTTAGAAAAA |
| | | | TAAACAAATAGGGGTTCCGCGCACATTTCCCCGAAAAGTG |
| | | | CCACCTGACGTCTAAGAAACCATTATTATCATGACATTAA |
| | | | CCTATAAAAATAGGCGTATCACGAGGCCCTTTCGTC |
| 55 | pYZZ437 | Rep2Cap5-p5. | TCGCGCGTTTCGGTGATGACGGTGAAAACCTCTGA |
| | | p5 | CACATGCAGCTCCCGGAGACGGTCACAGCTTGTCT |
| | | | GTAAGCGGATGCCGGGAGCAGACAAGCCCGTCAG |
| | | | GGCGCGTCAGCGGGTGTTGGCGGGTGTCGGGGCTG |
| | | | GCTTAACTATGCGGCATCAGAGCAGATTGTACTGA |
| | | | GAGTGCACCATATGCGGTGTGAAATACCGCACAGA |
| | | | TGCGTAAGGAGAAAATACCGCATCAGGCGCCATTC |
| | | | GCCATTCAGGCTGCGCAACTGTTGGGAAGGGCGAT |
| | | | CGGTGCGGGCCTCTTCGCTATTACGCCAGCTGGCG |
| | | | AAAGGGGGATGTGCTGCAAGGCGATTAAGTTGGGT |
| | | | AACGCCAGGGTTTTCCCAGTCACGACGTTGTAAAA |
| | | | CGACGGCCAGTGAATTGACGCGTATTGGGATCGCG |
| | | | CAGCCGCCATGCCGGGGTTTTACGAGATTGTGATTAA |
| | | | GGTCCCCAGCGACCTTGACGAGCATCTGCCCGGCATT |
| | | | TCTGACAGCTTTGTGAACTGGGTGGCCGAGAAGGAAT |
| | | | GGGAGTTGCCGCCAGATTCTGACATGGATCTGAATCT |
| | | | GATTGAGCAGGCACCCCTGACCGTGGCCGAGAAGCT |
| | | | GCAGCGCGACTTTCTGACGGAATGGCGCCGTGTGAGT |
| | | | AAGGCCCCGGAGGCTCTTTTCTTTGTGCAATTTGAGAA |
| | | | GGGAGAGAGCTACTTCCACATGCACGTGCTCGTGGAA |
| | | | ACCACCGGGGTGAAATCCATGGTTTTGGGACGTTTCCT |
| | | | GAGTCAGATTCGCGAAAAACTGATTCAGAGAATTTACC |
| | | | GCGGGATCGAGCCGACTTTGCCAAACTGGTTCGCGGT |
| | | | CACAAAGACCAGAAATGGCGCCGGAGGCGGGAACAA |
| | | | GGTGGTGGATGAGTGCTACATCCCCAATTACTTGCTCC |
| | | | CCAAAACCCAGCCTGAGCTCCAGTGGGCGTGGACTAA |
| | | | TATGGAACAGTATTTAAGCGCCTGTTTGAATCTCACGG |
| | | | AGCGTAAACGGTTGGTGGCGCAGCATCTGACGCACGT |
| | | | GTCGCAGACGCAGGAGCAGAACAAAGAGAATCAGAAT |
| | | | CCCAATTCTGATGCGCCGGTGATCAGATCAAAAACTTC |
| | | | AGCCAGGTACATGGAGCTGGTCGGGTGGCTCGTGGA |
| | | | CAAGGGGATTACCTCGGAGAAGCAGTGGATCCAGGAG |
| | | | GACCAGGCCTCATACATCTCCTTCAATGCGGCCTCCAA |
| | | | CTCGCGGTCCCAAATCAAGGCTGCCTTGGACAATGCG |
| | | | GGAAAGATTATGAGCCTGACTAAAACCGCCCCCGACT |
| | | | ACCTGGTGGGCCAGCAGCCCGTGGAGGACATTTCCAG |
| | | | CAATCGGATTTATAAAATTTTGGAACTAAACGGGTACG |
| | | | ATCCCCAATATGCGGCTTCCGTCTTTCTGGGATGGGC |
| | | | CACGAAAAAGTTCGGCAAGAGGAACACCATCTGGCTG |
| | | | TTTGGGCCTGCAACTACCGGGAAGACCAACATCGCGG |
| | | | AGGCCATAGCCCACACTGTGCCCTTCTACGGGTGCGT |
| | | | AAACTGGACCAATGAGAACTTTCCCTTCAACGACTGTG |
| | | | TCGACAAGATGGTGATCTGGTGGGAGGAGGGGAAGAT |
| | | | GACCGCCAAGGTCGTGGAGTCGGCCAAAGCCATTCTC |
| | | | GGAGGAAGCAAGGTGCGCGTGGACCAGAAATGCAAG |
| | | | TCCTCGGCCCAGATAGACCCGACTCCCGTGATCGTCA |
| | | | CCTCCAACACCAACATGTGCGCCGTGATTGACGGGAA |
| | | | CTCAACGACCTTCGAACACCAGCAGCCGTTGCAAGAC |
| | | | CGGATGTTCAAATTTGAACTCACCCGCCGTCTGGATCA |
| | | | TGACTTTGGGAAGGTCACCAAGCAGGAAGTCAAAGAC |
| | | | TTTTTCCGGTGGGCAAAGGATCACGTGGTTGAGGTGG |
| | | | AGCATGAATTCTACGTCAAAAAGGGTGGAGCCAAGAAA |
| | | | AGACCCGCCCCCAGTGACGCAGATATAAGTGAGCCCA |
| | | | AACGGGTGCGCGAGTCAGTTGCGCAGCCATCGACGTC |
| | | | AGACGCGGAAGCTTCGATCAACTACGCAGACAGGTAC |
| | | | CAAAACAAATGTTCTCGTCACGTGGGCATGAATCTGAT |
| | | | GCTGTTTCCCTGCAGACAATGCGAGAGAATGAATCAGA |
| | | | ATTCAAATATCTGCTTCACTCACGGACAGAAAGACTGT |
| | | | TTAGAGTGCTTTCCCGTGTCAGAATCTCAACCCGTTTC |
| | | | TGTCGTCAAAAAGGCGTATCAGAAACTGTGCTACATTC |
| | | | ATCATATCATGGGAAAGGTGCCAGACGCTTGCACTGC |
| | | | CTGCGATCTGGTCAATGTGGATTTGGATGACTGCATCT |
| | | | TTGAACAATAAATGATTTAAATCAGGTatgtcttttgttgatcac |
| | | | cctccagattggttggaagaagttggtgaaggtcttcgcgagtttttgggccttgaagc |
| | | | gggcccaccgaaaccaaaacccaatcagcagcatcaagatcaagcccgtggtcttg |
| | | | tgctgcctggttataactatctcggacccggaaacggtctcgatcgaggagagcctgt |
| | | | caacagggcagacgaggtcgcgcgagagcacgacatctcgtacaacgagcagctt |
| | | | gaggcgggagacaacccctacctcaagtacaaccacgcggacgccgagtttcagg |
| | | | agaagctcgccgacgacacatccttcgggggaaacctcggaaaggcagtctttcag |
| | | | gccaagaaaagggttctcgaaccttttggcctggttgaagagggtgctaagacggcc |
| | | | cctaccggaaagcggatagacgaccactttccaaaaagaaagaaggctcggaccg |
| | | | aagaggactccaagccttccacctcgtcagacgccgaagctggacccagcggatc |
| | | | ccagcagctgcaaatcccagcccaaccagcctcaagtttgggagctgatacaatgtc |
| | | | tgcgggaggtggcggcccattgggcgacaataaccaaggtgccgatggagtggg |
| | | | caatgcctcgggagattggcattgcgattccacgtggatgggggacagagtcgtca |
| | | | ccaagtccacccgaacctgggtgctgcccagctacaacaaccaccagtaccgaga |
| | | | gatcaaaagcggctccgtcgacggaagcaacgccaacgcctactttggatacagca |
| | | | ccccctgggggtactttgactttaaccgcttccacagccactggagcccccgagact |
| | | | ggcaaagactcatcaacaactactggggcttcagaccccggtccctcagagtcaaa |
| | | | atcttcaacattcaagtcaaagaggtcacggtgcaggactccaccaccaccatcgcc |
| | | | aacaacctcacctccaccgtccaagtgtttacggacgacgactaccagctgccctac |
| | | | gtcgtcggcaacgggaccgagggatgcctgccggccttccctccgcaggtctttac |
| | | | gctgccgcagtacggttacgcgacgctgaaccgcgacaacacagaaaatcccacc |
| | | | gagaggagcagcttcttctgcctagagtactttcccagcaagatgctgagaacgggc |
| | | | aacaactttgagtttacctacaactttgaggaggtgcccttccactccagcttcgctccc |
| | | | agtcagaacctgttcaagctggccaacccgctggtggaccagtacttgtaccgcttc |
| | | | gtgagcacaaataacactggcggagtccagttcaacaagaacctggccgggagat |
| | | | acgccaacacctacaaaaactggttcccggggcccatgggccgaacccagggctg |
| | | | gaacctgggctccggggtcaaccgcgccagtgtcagcgccttcgccacgaccaat |
| | | | aggatggagctcgagggcgcgagttaccaggtgcccccgcagccgaacggcatg |
| | | | accaacaacctccagggcagcaacacctatgccctggagaacactatgatcttcaac |
| | | | agccagccggcgaacccgggcaccaccgccacgtacctcgagggcaacatgctc |
| | | | atcaccagcgagagcgagacgcagccggtgaaccgcgtggcgtacaacgtcggc |
| | | | gggcagatggccaccaacaaccagagctccaccactgcccccgcgaccggcacg |
| | | | tacaacctccaggaaatcgtgcccggcagcgtgtggatggagagggacgtgtacct |
| | | | ccaaggacccatctgggccaagatcccagagacgggggcgcactttcacccctctc |
| | | | cggccatgggcggattcggactcaaacacccaccgcccatgatgctcatcaagaac |
| | | | acgcctgtgcccggaaatatcaccagcttctcggacgtgcccgtcagcagcttcatc |
| | | | acccagtacagcaccgggcaggtcaccgtggagatggagtgggagctcaagaag |
| | | | gaaaactccaagaggtggaacccagagatccagtacacaaacaactacaacgacc |
| | | | cccagtttgtggactttgccccggacagcaccggggaatacagaaccaccagacct |
| | | | atcggaacccgataccttacccgacccctttaaTTGCTTGTTAATCAA |
| | | | TAAACCGTTTAATTCGTTTCAGTTGAACTTTGGTC |
| | | | TCTGCCCTAGGGGAGGTCCTGTATTAGAGGTCACG |
| | | | TGAGTGTTTTGCGACATTTTGCGACACCATGTGGTC |
| | | | ACGCTGGGTATTTAAGCCCGAGTGAGCACGCAGGG |
| | | | TCTCCATTTTGAAGCGGGAGGTTTGAAcgcgcagccgcca |
| | | | agccgtttcagttgaactttggtctctgcgtatttctttcttatctagtttccatggaggt |
| | | | cctgtattagaggtcacgtgagtgttttgcgacattttgcgacaccatgtggtcacgctgg |
| | | | gtatttaagcccgagtgagcacgcagggtctccattttgaagcgggaggtttgaaA |
| | | | TCCCAATGGCGCGCCGAGCTTGGCTCGAGCATGGT |
| | | | CATAGCTGTTTCCTGTGTGAAATTGTTATCCGCTCA |
| | | | CAATTCCACACAACATACGAGCCGGAAGCATAAAG |
| | | | TGTAAAGCCTGGGGTGCCTAATGAGTGAGCTAACT |
| | | | CACATTAATTGCGTTGCGCTCACTGCCCGCTTTCCA |
| | | | GTCGGGAAACCTGTCGTGCCAGCTGCATTAATGAA |
| | | | TCGGCCAACGCGCGGGGAGAGGCGGTTTGCGTATT |
| | | | GGGCGCTCTTCCGCTTCCTCGCTCACTGACTCGCTG |
| | | | CGCTCGGTCGTTCGGCTGCGGCGAGCGGTATCAGC |
| | | | TCACTCAAAGGCGGTAATACGGTTATCCACAGAAT |
| | | | CAGGGGATAACGCAGGAAAGAACATGTGAGCAAA |
| | | | AGGCCAGCAAAAGGCCAGGAACCGTAAAAAGGCC |
| | | | GCGTTGCTGGCGTTTTTCCATAGGCTCCGCCCCCCT |
| | | | GACGAGCATCACAAAAATCGACGCTCAAGTCAGAG |
| | | | GTGGCGAAACCCGACAGGACTATAAAGATACCAG |
| | | | GCGTTTCCCCCTGGAAGCTCCCTCGTGCGCTCTCCT |
| | | | GTTCCGACCCTGCCGCTTACCGGATACCTGTCCGCC |
| | | | TTTCTCCCTTCGGGAAGCGTGGCGCTTTCTCATAGC |
| | | | TCACGCTGTAGGTATCTCAGTTCGGTGTAGGTCGTT |
| | | | CGCTCCAAGCTGGGCTGTGTGCACGAACCCCCCGT |
| | | | TCAGCCCGACCGCTGCGCCTTATCCGGTAACTATCG |
| | | | TCTTGAGTCCAACCCGGTAAGACACGACTTATCGC |
| | | | CACTGGCAGCAGCCACTGGTAACAGGATTAGCAGA |
| | | | GCGAGGTATGTAGGCGGTGCTACAGAGTTCTTGAA |
| | | | GTGGTGGCCTAACTACGGCTACACTAGAAGAACAG |
| | | | TATTTGGTATCTGCGCTCTGCTGAAGCCAGTTACCT |
| | | | TCGGAAAAAGAGTTGGTAGCTCTTGATCCGGCAAA |
| | | | CAAACCACCGCTGGTAGCGGTGGTTTTTTTGTTTGC |
| | | | AAGCAGCAGATTACGCGCAGAAAAAAAGGATCTC |
| | | | AAGAAGATCCTTTGATCTTTTCTACGGGGTCTGACG |
| | | | CTCAGTGGAACGAAAACTCACGTTAAGGGATTTTG |
| | | | GTCATGAGATTATCAAAAAGGATCTTCACCTAGAT |
| | | | CCTTTTAAATTAAAAATGAAGTTTTAAATCAATCTA |
| | | | AAGTATATATGAGTAAACTTGGTCTGACAGTTAGA |
| | | | AAAACTCATCGAGCATCAAATGAAACTGCAATTTA |
| | | | TTCATATCAGGATTATCAATACCATATTTTTGAAAA |
| | | | AGCCGTTTCTGTAATGAAGGAGAAAACTCACCGAG |
| | | | GCAGTTCCATAGGATGGCAAGATCCTGGTATCGGT |
| | | | CTGCGATTCCGACTCGTCCAACATCAATACAACCT |
| | | | ATTAATTTCCCCTCGTCAAAAATAAGGTTATCAAGT |
| | | | GAGAAATCACCATGAGTGACGACTGAATCCGGTGA |
| | | | GAATGGCAAAAGTTTATGCATTTCTTTCCAGACTTG |
| | | | TTCAACAGGCCAGCCATTACGCTCGTCATCAAAAT |
| | | | CACTCGCATCAACCAAACCGTTATTCATTCGTGATT |
| | | | GCGCCTGAGCGAGACGAAATACGCGATCGCTGTTA |
| | | | AAAGGACAATTACAAACAGGAATCGAATGCAACC |
| | | | GGCGCAGGAACACTGCCAGCGCATCAACAATATTT |
| | | | TCACCTGAATCAGGATATTCTTCTAATACCTGGAAT |
| | | | GCTGTTTTCCCAGGGATCGCAGTGGTGAGTAACCA |
| | | | TGCATCATCAGGAGTACGGATAAAATGCTTGATGG |
| | | | TCGGAAGAGGCATAAATTCCGTCAGCCAGTTTAGT |
| | | | CTGACCATCTCATCTGTAACATCATTGGCAACGCTA |
| | | | CCTTTGCCATGTTTCAGAAACAACTCTGGCGCATCG |
| | | | GGCTTCCCATACAATCGATAGATTGTCGCACCTGAT |
| | | | TGCCCGACATTATCGCGAGCCCATTTATACCCATAT |
| | | | AAATCAGCATCCATGTTGGAATTTAATCGCGGCCT |
| | | | AGAGCAAGACGTTTCCCGTTGAATATGGCTCATAC |
| | | | TCTTCCTTTTTCAATATTATTGAAGCATTTATCAGG |
| | | | GTTATTGTCTCATGAGCGGATACATATTTGAATGTA |
| | | | TTTAGAAAAATAAACAAATAGGGGTTCCGCGCACA |
| | | | TTTCCCCGAAAAGTGCCACCTGACGTCTAAGAAAC |
| | | | CATTATTATCATGACATTAACCTATAAAAATAGGC |
| | | | GTATCACGAGGCCCTTTCGTC |
| 56 | pYZZ444 | Rep2Cap5-p5. | TCGCGCGTTTCGGTGATGACGGTGAAAACCTCTGA |
| | | p5 | CACATGCAGCTCCCGGAGACGGTCACAGCTTGTCT |
| | | | GTAAGCGGATGCCGGGAGCAGACAAGCCCGTCAG |
| | | | GGCGCGTCAGCGGGTGTTGGCGGGTGTCGGGGCTG |
| | | | GCTTAACTATGCGGCATCAGAGCAGATTGTACTGA |
| | | | GAGTGCACCATATGCGGTGTGAAATACCGCACAGA |
| | | | TGCGTAAGGAGAAAATACCGCATCAGGCGCCATTC |
| | | | GCCATTCAGGCTGCGCAACTGTTGGGAAGGGCGAT |
| | | | CGGTGCGGGCCTCTTCGCTATTACGCCAGCTGGCG |
| | | | AAAGGGGGATGTGCTGCAAGGCGATTAAGTTGGGT |
| | | | AACGCCAGGGTTTTCCCAGTCACGACGTTGTAAAA |
| | | | CGACGGCCAGTGAATTGACGCGTATTGGGATCGCG |
| | | | CAGCCGCCATGCCGGGGTTTTACGAGATTGTGATTAA |
| | | | GGTCCCCAGCGACCTTGACGAGCATCTGCCCGGCATT |
| | | | TCTGACAGCTTTGTGAACTGGGTGGCCGAGAAGGAAT |
| | | | GGGAGTTGCCGCCAGATTCTGACATGGATCTGAATCT |
| | | | GATTGAGCAGGCACCCCTGACCGTGGCCGAGAAGCT |
| | | | GCAGCGCGACTTTCTGACGGAATGGCGCCGTGTGAGT |
| | | | AAGGCCCCGGAGGCTCTTTTCTTTGTGCAATTTGAGAA |
| | | | GGGAGAGAGCTACTTCCACATGCACGTGCTCGTGGAA |
| | | | ACCACCGGGGTGAAATCCATGGTTTTGGGACGTTTCCT |
| | | | GAGTCAGATTCGCGAAAAACTGATTCAGAGAATTTACC |
| | | | GCGGGATCGAGCCGACTTTGCCAAACTGGTTCGCGGT |
| | | | CACAAAGACCAGAAATGGCGCCGGAGGCGGGAACAA |
| | | | GGTGGTGGATGAGTGCTACATCCCCAATTACTTGCTCC |
| | | | CCAAAACCCAGCCTGAGCTCCAGTGGGCGTGGACTAA |
| | | | TATGGAACAGTATTTAAGCGCCTGTTTGAATCTCACGG |
| | | | AGCGTAAACGGTTGGTGGCGCAGCATCTGACGCACGT |
| | | | GTCGCAGACGCAGGAGCAGAACAAAGAGAATCAGAAT |
| | | | CCCAATTCTGATGCGCCGGTGATCAGATCAAAAACTTC |
| | | | AGCCAGGTACATGGAGCTGGTCGGGTGGCTCGTGGA |
| | | | CAAGGGGATTACCTCGGAGAAGCAGTGGATCCAGGAG |
| | | | GACCAGGCCTCATACATCTCCTTCAATGCGGCCTCCAA |
| | | | CTCGCGGTCCCAAATCAAGGCTGCCTTGGACAATGCG |
| | | | GGAAAGATTATGAGCCTGACTAAAACCGCCCCCGACT |
| | | | ACCTGGTGGGCCAGCAGCCCGTGGAGGACATTTCCAG |
| | | | CAATCGGATTTATAAAATTTTGGAACTAAACGGGTACG |
| | | | ATCCCCAATATGCGGCTTCCGTCTTTCTGGGATGGGC |
| | | | CACGAAAAAGTTCGGCAAGAGGAACACCATCTGGCTG |
| | | | TTTGGGCCTGCAACTACCGGGAAGACCAACATCGCGG |
| | | | AGGCCATAGCCCACACTGTGCCCTTCTACGGGTGCGT |
| | | | AAACTGGACCAATGAGAACTTTCCCTTCAACGACTGTG |
| | | | TCGACAAGATGGTGATCTGGTGGGAGGAGGGGAAGAT |
| | | | GACCGCCAAGGTCGTGGAGTCGGCCAAAGCCATTCTC |
| | | | GGAGGAAGCAAGGTGCGCGTGGACCAGAAATGCAAG |
| | | | TCCTCGGCCCAGATAGACCCGACTCCCGTGATCGTCA |
| | | | CCTCCAACACCAACATGTGCGCCGTGATTGACGGGAA |
| | | | CTCAACGACCTTCGAACACCAGCAGCCGTTGCAAGAC |
| | | | CGGATGTTCAAATTTGAACTCACCCGCCGTCTGGATCA |
| | | | TGACTTTGGGAAGGTCACCAAGCAGGAAGTCAAAGAC |
| | | | TTTTTCCGGTGGGCAAAGGATCACGTGGTTGAGGTGG |
| | | | AGCATGAATTCTACGTCAAAAAGGGTGGAGCCAAGAAA |
| | | | AGACCCGCCCCCAGTGACGCAGATATAAGTGAGCCCA |
| | | | AACGGGTGCGCGAGTCAGTTGCGCAGCCATCGACGTC |
| | | | AGACGCGGAAGCTTCGATCAACTACGCAGACAGGTAC |
| | | | CAAAACAAATGTTCTCGTCACGTGGGCATGAATCTGAT |
| | | | GCTGTTTCCCTGCAGACAATGCGAGAGAATGAATCAGA |
| | | | ATTCAAATATCTGCTTCACTCACGGACAGAAAGACTGT |
| | | | TTAGAGTGCTTTCCCGTGTCAGAATCTCAACCCGTTTC |
| | | | TGTCGTCAAAAAGGCGTATCAGAAACTGTGCTACATTC |
| | | | ATCATATCATGGGAAAGGTGCCAGACGCTTGCACTGC |
| | | | CTGCGATCTGGTCAATGTGGATTTGGATGACTGCATCT |
| | | | TTGAACAATAAATGATTTAAATCAGGTatgtcttttgttgatcac |
| | | | cctccagattggttggaagaagttggtgaaggtcttcgcgagtttttgggccttgaagc |
| | | | gggcccaccgaaaccaaaacccaatcagcagcatcaagatcaagcccgtggtcttg |
| | | | tgctgcctggttataactatctcggacccggaaacggtctcgatcgaggagagcctgt |
| | | | caacagggcagacgaggtcgcgcgagagcacgacatctcgtacaacgagcagctt |
| | | | gaggcgggagacaacccctacctcaagtacaaccacgcggacgccgagtttcagg |
| | | | agaagctcgccgacgacacatccttcgggggaaacctcggaaaggcagtctttcag |
| | | | gccaagaaaagggttctcgaaccttttggcctggttgaagagggtgctaagacggcc |
| | | | cctaccggaaagcggatagacgaccactttccaaaaagaaagaaggctcggaccg |
| | | | aagaggactccaagccttccacctcgtcagacgccgaagctggacccagcggatc |
| | | | ccagcagctgcaaatcccagcccaaccagcctcaagtttgggagctgatacaatgtc |
| | | | tgcgggaggtggcggcccattgggcgacaataaccaaggtgccgatggagtggg |
| | | | caatgcctcgggagattggcattgcgattccacgtggatgggggacagagtcgtca |
| | | | ccaagtccacccgaacctgggtgctgcccagctacaacaaccaccagtaccgaga |
| | | | gatcaaaagcggctccgtcgacggaagcaacgccaacgcctactttggatacagca |
| | | | ccccctgggggtactttgactttaaccgcttccacagccactggagcccccgagact |
| | | | ggcaaagactcatcaacaactactggggcttcagaccccggtccctcagagtcaaa |
| | | | atcttcaacattcaagtcaaagaggtcacggtgcaggactccaccaccaccatcgcc |
| | | | aacaacctcacctccaccgtccaagtgtttacggacgacgactaccagctgccctac |
| | | | gtcgtcggcaacgggaccgagggatgcctgccggccttccctccgcaggtctttac |
| | | | gctgccgcagtacgcttacgcgacgctgaaccgcgacaacacagaaaatcccacc |
| | | | gagaggagcagcttcttctgcctagagtactttcccagcaagatgctgagaacgggc |
| | | | aacaactttgagtttacctacaactttgaggaggtgcccttccactccagcttcgctccc |
| | | | agtcagaacctgttcaagctggccaacccgctggtggaccagtacttgtaccgcttc |
| | | | gtgagcacaaataacactggcggagtccagttcaacaagaacctggccgggagat |
| | | | acgccaacacctacaaaaactggttcccggggcccatgggccgaacccagggctg |
| | | | gaacctgggctccggggtcaaccgcgccagtgtcagcgccttcgccacgaccaat |
| | | | aggatggagctcgagggcgcgagttaccaggtgcccccgcagccgaacggcatg |
| | | | accaacaacctccagggcagcaacacctatgccctggagaacactatgatcttcaac |
| | | | agccagccggcgaacccgggcaccaccgccacgtacctcgagggcaacatgctc |
| | | | atcaccagcgagagcgagacgcagccggtgaaccgcgtggcgtacaacgtcggc |
| | | | gggcagatggccaccaacaaccagagctccaccactgcccccgcgaccggcacg |
| | | | tacaacctccaggaaatcgtgcccggcagcgtgtggatggagagggacgtgtacct |
| | | | ccaaggacccatctgggccaagatcccagagacgggggcgcactttcacccctctc |
| | | | cggccatgggcggattcggactcaaacacccaccgcccatgatgctcatcaagaac |
| | | | acgcctgtgcccggaaatatcaccagcttctcggacgtgcccgtcagcagcttcatc |
| | | | acccagtacagcaccgggcaggtcaccgtggagatggagtgggagctcaagaag |
| | | | gaaaactccaagaggtggaacccagagatccagtacacaaacaactacaacgacc |
| | | | cccagtttgtggactttgccccggacagcaccggggaatacagaaccaccagacct |
| | | | atcggaacccgataccttacccgacccctttaaTTGCTTGTTAATCAA |
| | | | TAAACCGTTTAATTCGTTTCAGTTGAACTTTGGTC |
| | | | TCTGCCCTAGGGGAGGTCCTGTATTAGAGGTCAC |
| | | | GTGAGTGTTTTGCGACATTTTGCGACACCATGT |
| | | | GGTCACGCTGGGTATTTAAGCCCGAGTGAGCAC |
| | | | GCAGGGTCTCCATTTTGAAGCGGGAGGTTTGAA |
| | | | GTCTTACCCCCAATAAGGGGATTTGCTCTATTTAAT |
| | | | TAGGAATAAGGTCGATTACTGATAGAACAAATCCA |
| | | | GGCTACTGTGTTTAGTAATCAGATTTGTTgaggtcctgta |
| | | | ttagaggtcacgtgagtgttttgcgacattttgcgacaccatgtggtcacgctgg |
| | | | gtatttaagcccgagtgagcacgcagggtctccattttgaagcgggaggtttga |
| | | | aATCCCAATGGCGCGCCGAGCTTGGCTCGAGCATG |
| | | | GTCATAGCTGTTTCCTGTGTGAAATTGTTATCCGCT |
| | | | CACAATTCCACACAACATACGAGCCGGAAGCATAA |
| | | | AGTGTAAAGCCTGGGGTGCCTAATGAGTGAGCTAA |
| | | | CTCACATTAATTGCGTTGCGCTCACTGCCCGCTTTC |
| | | | CAGTCGGGAAACCTGTCGTGCCAGCTGCATTAATG |
| | | | AATCGGCCAACGCGCGGGGAGAGGCGGTTTGCGTA |
| | | | TTGGGCGCTCTTCCGCTTCCTCGCTCACTGACTCGC |
| | | | TGCGCTCGGTCGTTCGGCTGCGGCGAGCGGTATCA |
| | | | GCTCACTCAAAGGCGGTAATACGGTTATCCACAGA |
| | | | ATCAGGGGATAACGCAGGAAAGAACATGTGAGCA |
| | | | AAAGGCCAGCAAAAGGCCAGGAACCGTAAAAAGG |
| | | | CCGCGTTGCTGGCGTTTTTCCATAGGCTCCGCCCCC |
| | | | CTGACGAGCATCACAAAAATCGACGCTCAAGTCAG |
| | | | AGGTGGCGAAACCCGACAGGACTATAAAGATACC |
| | | | AGGCGTTTCCCCCTGGAAGCTCCCTCGTGCGCTCTC |
| | | | CTGTTCCGACCCTGCCGCTTACCGGATACCTGTCCG |
| | | | CCTTTCTCCCTTCGGGAAGCGTGGCGCTTTCTCATA |
| | | | GCTCACGCTGTAGGTATCTCAGTTCGGTGTAGGTCG |
| | | | TTCGCTCCAAGCTGGGCTGTGTGCACGAACCCCCC |
| | | | GTTCAGCCCGACCGCTGCGCCTTATCCGGTAACTAT |
| | | | CGTCTTGAGTCCAACCCGGTAAGACACGACTTATC |
| | | | GCCACTGGCAGCAGCCACTGGTAACAGGATTAGCA |
| | | | GAGCGAGGTATGTAGGCGGTGCTACAGAGTTCTTG |
| | | | AAGTGGTGGCCTAACTACGGCTACACTAGAAGAAC |
| | | | AGTATTTGGTATCTGCGCTCTGCTGAAGCCAGTTAC |
| | | | CTTCGGAAAAAGAGTTGGTAGCTCTTGATCCGGCA |
| | | | AACAAACCACCGCTGGTAGCGGTGGTTTTTTTGTTT |
| | | | GCAAGCAGCAGATTACGCGCAGAAAAAAAGGATC |
| | | | TCAAGAAGATCCTTTGATCTTTTCTACGGGGTCTGA |
| | | | CGCTCAGTGGAACGAAAACTCACGTTAAGGGATTT |
| | | | TGGTCATGAGATTATCAAAAAGGATCTTCACCTAG |
| | | | ATCCTTTTAAATTAAAAATGAAGTTTTAAATCAATC |
| | | | TAAAGTATATATGAGTAAACTTGGTCTGACAGTTA |
| | | | GAAAAACTCATCGAGCATCAAATGAAACTGCAATT |
| | | | TATTCATATCAGGATTATCAATACCATATTTTTGAA |
| | | | AAAGCCGTTTCTGTAATGAAGGAGAAAACTCACCG |
| | | | AGGCAGTTCCATAGGATGGCAAGATCCTGGTATCG |
| | | | GTCTGCGATTCCGACTCGTCCAACATCAATACAAC |
| | | | CTATTAATTTCCCCTCGTCAAAAATAAGGTTATCAA |
| | | | GTGAGAAATCACCATGAGTGACGACTGAATCCGGT |
| | | | GAGAATGGCAAAAGTTTATGCATTTCTTTCCAGACT |
| | | | TGTTCAACAGGCCAGCCATTACGCTCGTCATCAAA |
| | | | ATCACTCGCATCAACCAAACCGTTATTCATTCGTGA |
| | | | TTGCGCCTGAGCGAGACGAAATACGCGATCGCTGT |
| | | | TAAAAGGACAATTACAAACAGGAATCGAATGCAA |
| | | | CCGGCGCAGGAACACTGCCAGCGCATCAACAATAT |
| | | | TTTCACCTGAATCAGGATATTCTTCTAATACCTGGA |
| | | | ATGCTGTTTTCCCAGGGATCGCAGTGGTGAGTAAC |
| | | | CATGCATCATCAGGAGTACGGATAAAATGCTTGAT |
| | | | GGTCGGAAGAGGCATAAATTCCGTCAGCCAGTTTA |
| | | | GTCTGACCATCTCATCTGTAACATCATTGGCAACGC |
| | | | TACCTTTGCCATGTTTCAGAAACAACTCTGGCGCAT |
| | | | CGGGCTTCCCATACAATCGATAGATTGTCGCACCT |
| | | | GATTGCCCGACATTATCGCGAGCCCATTTATACCCA |
| | | | TATAAATCAGCATCCATGTTGGAATTTAATCGCGG |
| | | | CCTAGAGCAAGACGTTTCCCGTTGAATATGGCTCA |
| | | | TACTCTTCCTTTTTCAATATTATTGAAGCATTTATCA |
| | | | GGGTTATTGTCTCATGAGCGGATACATATTTGAATG |
| | | | TATTTAGAAAAATAAACAAATAGGGGTTCCGCGCA |
| | | | CATTTCCCCGAAAAGTGCCACCTGACGTCTAAGAA |
| | | | ACCATTATTATCATGACATTAACCTATAAAAATAG |
| | | | GCGTATCACGAGGCCCTTTCGTC |
| 57 | pYZZ445 | Rep2Cap5-p5.p5 | TCGCGCGTTTCGGTGATGACGGTGAAAACCTCTGA |
| | | | CACATGCAGCTCCCGGAGACGGTCACAGCTTGTCT |
| | | | GTAAGCGGATGCCGGGAGCAGACAAGCCCGTCAG |
| | | | GGCGCGTCAGCGGGTGTTGGCGGGTGTCGGGGCTG |
| | | | GCTTAACTATGCGGCATCAGAGCAGATTGTACTGA |
| | | | GAGTGCACCATATGCGGTGTGAAATACCGCACAGA |
| | | | TGCGTAAGGAGAAAATACCGCATCAGGCGCCATTC |
| | | | GCCATTCAGGCTGCGCAACTGTTGGGAAGGGCGAT |
| | | | CGGTGCGGGCCTCTTCGCTATTACGCCAGCTGGCG |
| | | | AAAGGGGGATGTGCTGCAAGGCGATTAAGTTGGGT |
| | | | AACGCCAGGGTTTTCCCAGTCACGACGTTGTAAAA |
| | | | CGACGGCCAGTGAATTGACGCGTATTGGGATCGCG |
| | | | CAGCCGCCATGCCGGGGTTTTACGAGATTGTGATTAA |
| | | | GGTCCCCAGCGACCTTGACGAGCATCTGCCCGGCATT |
| | | | TCTGACAGCTTTGTGAACTGGGTGGCCGAGAAGGAAT |
| | | | GGGAGTTGCCGCCAGATTCTGACATGGATCTGAATCT |
| | | | GATTGAGCAGGCACCCCTGACCGTGGCCGAGAAGCT |
| | | | GCAGCGCGACTTTCTGACGGAATGGCGCCGTGTGAGT |
| | | | AAGGCCCCGGAGGCTCTTTTCTTTGTGCAATTTGAGAA |
| | | | GGGAGAGAGCTACTTCCACATGCACGTGCTCGTGGAA |
| | | | ACCACCGGGGTGAAATCCATGGTTTTGGGACGTTTCCT |
| | | | GAGTCAGATTCGCGAAAAACTGATTCAGAGAATTTACC |
| | | | GCGGGATCGAGCCGACTTTGCCAAACTGGTTCGCGGT |
| | | | CACAAAGACCAGAAATGGCGCCGGAGGCGGGAACAA |
| | | | GGTGGTGGATGAGTGCTACATCCCCAATTACTTGCTCC |
| | | | CCAAAACCCAGCCTGAGCTCCAGTGGGCGTGGACTAA |
| | | | TATGGAACAGTATTTAAGCGCCTGTTTGAATCTCACGG |
| | | | AGCGTAAACGGTTGGTGGCGCAGCATCTGACGCACGT |
| | | | GTCGCAGACGCAGGAGCAGAACAAAGAGAATCAGAAT |
| | | | CCCAATTCTGATGCGCCGGTGATCAGATCAAAAACTTC |
| | | | AGCCAGGTACATGGAGCTGGTCGGGTGGCTCGTGGA |
| | | | CAAGGGGATTACCTCGGAGAAGCAGTGGATCCAGGAG |
| | | | GACCAGGCCTCATACATCTCCTTCAATGCGGCCTCCAA |
| | | | CTCGCGGTCCCAAATCAAGGCTGCCTTGGACAATGCG |
| | | | GGAAAGATTATGAGCCTGACTAAAACCGCCCCCGACT |
| | | | ACCTGGTGGGCCAGCAGCCCGTGGAGGACATTTCCAG |
| | | | CAATCGGATTTATAAAATTTTGGAACTAAACGGGTACG |
| | | | ATCCCCAATATGCGGCTTCCGTCTTTCTGGGATGGGC |
| | | | CACGAAAAAGTTCGGCAAGAGGAACACCATCTGGCTG |
| | | | TTTGGGCCTGCAACTACCGGGAAGACCAACATCGCGG |
| | | | AGGCCATAGCCCACACTGTGCCCTTCTACGGGTGCGT |
| | | | AAACTGGACCAATGAGAACTTTCCCTTCAACGACTGTG |
| | | | TCGACAAGATGGTGATCTGGTGGGAGGAGGGGAAGAT |
| | | | GACCGCCAAGGTCGTGGAGTCGGCCAAAGCCATTCTC |
| | | | GGAGGAAGCAAGGTGCGCGTGGACCAGAAATGCAAG |
| | | | TCCTCGGCCCAGATAGACCCGACTCCCGTGATCGTCA |
| | | | CCTCCAACACCAACATGTGCGCCGTGATTGACGGGAA |
| | | | CTCAACGACCTTCGAACACCAGCAGCCGTTGCAAGAC |
| | | | CGGATGTTCAAATTTGAACTCACCCGCCGTCTGGATCA |
| | | | TGACTTTGGGAAGGTCACCAAGCAGGAAGTCAAAGAC |
| | | | TTTTTCCGGTGGGCAAAGGATCACGTGGTTGAGGTGG |
| | | | AGCATGAATTCTACGTCAAAAAGGGTGGAGCCAAGAAA |
| | | | AGACCCGCCCCCAGTGACGCAGATATAAGTGAGCCCA |
| | | | AACGGGTGCGCGAGTCAGTTGCGCAGCCATCGACGTC |
| | | | AGACGCGGAAGCTTCGATCAACTACGCAGACAGGTAC |
| | | | CAAAACAAATGTTCTCGTCACGTGGGCATGAATCTGAT |
| | | | GCTGTTTCCCTGCAGACAATGCGAGAGAATGAATCAGA |
| | | | ATTCAAATATCTGCTTCACTCACGGACAGAAAGACTGT |
| | | | TTAGAGTGCTTTCCCGTGTCAGAATCTCAACCCGTTTC |
| | | | TGTCGTCAAAAAGGCGTATCAGAAACTGTGCTACATTC |
| | | | ATCATATCATGGGAAAGGTGCCAGACGCTTGCACTGC |
| | | | CTGCGATCTGGTCAATGTGGATTTGGATGACTGCATCT |
| | | | TTGAACAATAAATGATTTAAATCAGGTatgtcttttgttgatcac |
| | | | cctccagattggttggaagaagttggtgaaggtcttcgcgagtttttgggccttgaagc |
| | | | gggcccaccgaaaccaaaacccaatcagcagcatcaagatcaagcccgtggtcttg |
| | | | tgctgcctggttataactatctcggacccggaaacggtctcgatcgaggagagcctgt |
| | | | caacagggcagacgaggtcgcgcgagagcacgacatctcgtacaacgagcagctt |
| | | | gaggcgggagacaacccctacctcaagtacaaccacgcggacgccgagtttcagg |
| | | | agaagctcgccgacgacacatccttcgggggaaacctcggaaaggcagtctttcag |
| | | | gccaagaaaagggttctcgaaccttttggcctggttgaagagggtcctaagacggcc |
| | | | cctaccggaaagcggatagacgaccactttccaaaaagaaagaaggctcggaccg |
| | | | aagaggactccaagccttccacctcgtcagacgccgaagctggacccagcggatc |
| | | | ccagcagctgcaaatcccagcccaaccagcctcaagtttgggagctgatacaatgtc |
| | | | tgcgggaggtggcggcccattgggcgacaataaccaaggtgccgatggagtggg |
| | | | caatgcctcgggagattggcattgcgattccacgtggatgggggacagagtcgtca |
| | | | ccaagtccacccgaacctgggtgctgcccagctacaacaaccaccagtaccgaga |
| | | | gatcaaaagcggctccgtcgacggaagcaacgccaacgcctactttggatacagca |
| | | | ccccctgggggtactttgactttaaccgcttccacagccactggagcccccgagact |
| | | | ggcaaagactcatcaacaactactggggcttcagaccccggtccctcagagtcaaa |
| | | | atcttcaacattcaagtcaaagaggtcacggtgcaggactccaccaccaccatcgcc |
| | | | aacaacctcacctccaccgtccaagtgtttacggacgacgactaccagctgccctac |
| | | | gtcgtcggcaacgggaccgagggatgcctgccggccttccctccgcaggtctttac |
| | | | gctgccgcagtacggttacgcgacgctgaaccgcgacaacacagaaaatcccacc |
| | | | gagaggagcagcttcttctgcctagagtactttcccagcaagatgctgagaacgggc |
| | | | aacaactttgagtttacctacaactttgaggaggtgcccttccactccagcttcgctccc |
| | | | agtcagaacctgttcaagctggccaacccgctggtggaccagtacttgtaccgcttc |
| | | | gtgagcacaaataacactggcggagtccagttcaacaagaacctggccgggagat |
| | | | acgccaacacctacaaaaactggttcccggggcccatgggccgaacccagggctg |
| | | | gaacctgggctccgggctcaaccgcgccagtctcagcgccttcgccacgaccaat |
| | | | aggatggagctcgagggcgcgagttaccaggtgcccccgcagccgaacggcatg |
| | | | accaacaacctccagggcagcaacacctatgccctggagaacactatgatcttcaac |
| | | | agccagccggcgaacccgggcaccaccgccacgtacctcgagggcaacatgctc |
| | | | atcaccagcgagagcgagacgcagccggtgaaccgcgtggcgtacaacgtcggc |
| | | | gggcagatggccaccaacaaccagagctccaccactgcccccgcgaccggcacg |
| | | | tacaacctccaggaaatcgtgcccggcagcgtgtggatggagagggacgtgtacct |
| | | | ccaaggacccatctgggccaagatcccagagacgggggcgcactttcacccctctc |
| | | | cggccatgggcggattcggactcaaacacccaccgcccatgatgctcatcaagaac |
| | | | acgcctgtgcccggaaatatcaccagcttctcggacgtgcccgtcagcagcttcatc |
| | | | acccagtacagcaccgggcaggtcaccgtggagatggagtgggagctcaagaag |
| | | | gaaaactccaagaggtggaacccagagatccagtacacaaacaactacaacgacc |
| | | | cccagtttgtggactttgccccggacagcaccggggaatacagaaccaccagacct |
| | | | atcggaacccgataccttacccgacccctttaaTTGCTTGTTAATCAA |
| | | | TAAACCGTTTAATTCGTTTCAGTTGAACTTTGGTC |
| | | | TCTGCCCTAGGGGAGGTCCTGTATTAGAGGTCAC |
| | | | GTGAGTGTTTTGCGACATTTTGCGACACCATGT |
| | | | GGTCACGCTGGGTATTTAAGCCCGAGTGAGCAC |
| | | | GCAGGGTCTCCATTTTGAAGCGGGAGGTTTGAA |
| | | | GTCTTACCCCCAATAAGGGGATTTGCTCTATTTAAT |
| | | | TAGGAATAAGGTCGATTACTGATAGAACAAATCCA |
| | | | GGCTACTGTGTTTAGTAATCAGATTTGTTCGTGACC |
| | | | GATATGCACGGGCAAAACGGCAGGAGGTTGTTAGC |
| | | | GCGACCTCCTGCCACCCGCTTTCACGAAGGTCATGT |
| | | | GTAAAAGGCCGCAGCGTAACTAgaggtcctgtattagaggtc |
| | | | acgtgagtgttttgcgacattttgcgacaccatgtggtcacgctgggtatttaagc |
| | | | ccgagtgagcacgcagggtctccattttgaagcgggaggtttgaaATCCCA |
| | | | ATGGCGCGCCGAGCTTGGCTCGAGCATGGTCATAG |
| | | | CTGTTTCCTGTGTGAAATTGTTATCCGCTCACAATT |
| | | | CCACACAACATACGAGCCGGAAGCATAAAGTGTAA |
| | | | AGCCTGGGGTGCCTAATGAGTGAGCTAACTCACAT |
| | | | TAATTGCGTTGCGCTCACTGCCCGCTTTCCAGTCGG |
| | | | GAAACCTGTCGTGCCAGCTGCATTAATGAATCGGC |
| | | | CAACGCGCGGGGAGAGGCGGTTTGCGTATTGGGCG |
| | | | CTCTTCCGCTTCCTCGCTCACTGACTCGCTGCGCTC |
| | | | GGTCGTTCGGCTGCGGCGAGCGGTATCAGCTCACT |
| | | | CAAAGGCGGTAATACGGTTATCCACAGAATCAGGG |
| | | | GATAACGCAGGAAAGAACATGTGAGCAAAAGGCC |
| | | | AGCAAAAGGCCAGGAACCGTAAAAAGGCCGCGTT |
| | | | GCTGGCGTTTTTCCATAGGCTCCGCCCCCCTGACGA |
| | | | GCATCACAAAAATCGACGCTCAAGTCAGAGGTGGC |
| | | | GAAACCCGACAGGACTATAAAGATACCAGGCGTTT |
| | | | CCCCCTGGAAGCTCCCTCGTGCGCTCTCCTGTTCCG |
| | | | ACCCTGCCGCTTACCGGATACCTGTCCGCCTTTCTC |
| | | | CCTTCGGGAAGCGTGGCGCTTTCTCATAGCTCACGC |
| | | | TGTAGGTATCTCAGTTCGGTGTAGGTCGTTCGCTCC |
| | | | AAGCTGGGCTGTGTGCACGAACCCCCCGTTCAGCC |
| | | | CGACCGCTGCGCCTTATCCGGTAACTATCGTCTTGA |
| | | | GTCCAACCCGGTAAGACACGACTTATCGCCACTGG |
| | | | CAGCAGCCACTGGTAACAGGATTAGCAGAGCGAG |
| | | | GTATGTAGGCGGTGCTACAGAGTTCTTGAAGTGGT |
| | | | GGCCTAACTACGGCTACACTAGAAGAACAGTATTT |
| | | | GGTATCTGCGCTCTGCTGAAGCCAGTTACCTTCGGA |
| | | | AAAAGAGTTGGTAGCTCTTGATCCGGCAAACAAAC |
| | | | CACCGCTGGTAGCGGTGGTTTTTTTGTTTGCAAGCA |
| | | | GCAGATTACGCGCAGAAAAAAAGGATCTCAAGAA |
| | | | GATCCTTTGATCTTTTCTACGGGGTCTGACGCTCAG |
| | | | TGGAACGAAAACTCACGTTAAGGGATTTTGGTCAT |
| | | | GAGATTATCAAAAAGGATCTTCACCTAGATCCTTTT |
| | | | AAATTAAAAATGAAGTTTTAAATCAATCTAAAGTA |
| | | | TATATGAGTAAACTTGGTCTGACAGTTAGAAAAAC |
| | | | TCATCGAGCATCAAATGAAACTGCAATTTATTCAT |
| | | | ATCAGGATTATCAATACCATATTTTTGAAAAAGCC |
| | | | GTTTCTGTAATGAAGGAGAAAACTCACCGAGGCAG |
| | | | TTCCATAGGATGGCAAGATCCTGGTATCGGTCTGC |
| | | | GATTCCGACTCGTCCAACATCAATACAACCTATTA |
| | | | ATTTCCCCTCGTCAAAAATAAGGTTATCAAGTGAG |
| | | | AAATCACCATGAGTGACGACTGAATCCGGTGAGAA |
| | | | TGGCAAAAGTTTATGCATTTCTTTCCAGACTTGTTC |
| | | | AACAGGCCAGCCATTACGCTCGTCATCAAAATCAC |
| | | | TCGCATCAACCAAACCGTTATTCATTCGTGATTGCG |
| | | | CCTGAGCGAGACGAAATACGCGATCGCTGTTAAAA |
| | | | GGACAATTACAAACAGGAATCGAATGCAACCGGC |
| | | | GCAGGAACACTGCCAGCGCATCAACAATATTTTCA |
| | | | CCTGAATCAGGATATTCTTCTAATACCTGGAATGCT |
| | | | GTTTTCCCAGGGATCGCAGTGGTGAGTAACCATGC |
| | | | ATCATCAGGAGTACGGATAAAATGCTTGATGGTCG |
| | | | GAAGAGGCATAAATTCCGTCAGCCAGTTTAGTCTG |
| | | | ACCATCTCATCTGTAACATCATTGGCAACGCTACCT |
| | | | TTGCCATGTTTCAGAAACAACTCTGGCGCATCGGG |
| | | | CTTCCCATACAATCGATAGATTGTCGCACCTGATTG |
| | | | CCCGACATTATCGCGAGCCCATTTATACCCATATAA |
| | | | ATCAGCATCCATGTTGGAATTTAATCGCGGCCTAG |
| | | | AGCAAGACGTTTCCCGTTGAATATGGCTCATACTCT |
| | | | TCCTTTTTCAATATTATTGAAGCATTTATCAGGGTT |
| | | | ATTGTCTCATGAGCGGATACATATTTGAATGTATTT |
| | | | AGAAAAATAAACAAATAGGGGTTCCGCGCACATTT |
| | | | CCCCGAAAAGTGCCACCTGACGTCTAAGAAACCAT |
| | | | TATTATCATGACATTAACCTATAAAAATAGGCGTA |
| | | | TCACGAGGCCCTTTCGTC |
| 58 | pYZZ446 | Rep2Cap5-p5. | TCGCGCGTTTCGGTGATGACGGTGAAAACCTCTGA |
| | | p5 | CACATGCAGCTCCCGGAGACGGTCACAGCTTGTCT |
| | | | GTAAGCGGATGCCGGGAGCAGACAAGCCCGTCAG |
| | | | GGCGCGTCAGCGGGTGTTGGCGGGTGTCGGGGCTG |
| | | | GCTTAACTATGCGGCATCAGAGCAGATTGTACTGA |
| | | | GAGTGCACCATATGCGGTGTGAAATACCGCACAGA |
| | | | TGCGTAAGGAGAAAATACCGCATCAGGCGCCATTC |
| | | | GCCATTCAGGCTGCGCAACTGTTGGGAAGGGCGAT |
| | | | CGGTGCGGGCCTCTTCGCTATTACGCCAGCTGGCG |
| | | | AAAGGGGGATGTGCTGCAAGGCGATTAAGTTGGGT |
| | | | AACGCCAGGGTTTTCCCAGTCACGACGTTGTAAAA |
| | | | CGACGGCCAGTGAATTGACGCGTATTGGGATCGCG |
| | | | CAGCCGCCATGCCGGGGTTTTACGAGATTGTGATTAA |
| | | | GGTCCCCAGCGACCTTGACGAGCATCTGCCCGGCATT |
| | | | TCTGACAGCTTTGTGAACTGGGTGGCCGAGAAGGAAT |
| | | | GGGAGTTGCCGCCAGATTCTGACATGGATCTGAATCT |
| | | | GATTGAGCAGGCACCCCTGACCGTGGCCGAGAAGCT |
| | | | GCAGCGCGACTTTCTGACGGAATGGCGCCGTGTGAGT |
| | | | AAGGCCCCGGAGGCTCTTTTCTTTGTGCAATTTGAGAA |
| | | | GGGAGAGAGCTACTTCCACATGCACGTGCTCGTGGAA |
| | | | ACCACCGGGGTGAAATCCATGGTTTTGGGACGTTTCCT |
| | | | GAGTCAGATTCGCGAAAAACTGATTCAGAGAATTTACC |
| | | | GCGGGATCGAGCCGACTTTGCCAAACTGGTTCGCGGT |
| | | | CACAAAGACCAGAAATGGCGCCGGAGGCGGGAACAA |
| | | | GGTGGTGGATGAGTGCTACATCCCCAATTACTTGCTCC |
| | | | CCAAAACCCAGCCTGAGCTCCAGTGGGCGTGGACTAA |
| | | | TATGGAACAGTATTTAAGCGCCTGTTTGAATCTCACGG |
| | | | AGCGTAAACGGTTGGTGGCGCAGCATCTGACGCACGT |
| | | | GTCGCAGACGCAGGAGCAGAACAAAGAGAATCAGAAT |
| | | | CCCAATTCTGATGCGCCGGTGATCAGATCAAAAACTTC |
| | | | AGCCAGGTACATGGAGCTGGTCGGGTGGCTCGTGGA |
| | | | CAAGGGGATTACCTCGGAGAAGCAGTGGATCCAGGAG |
| | | | GACCAGGCCTCATACATCTCCTTCAATGCGGCCTCCAA |
| | | | CTCGCGGTCCCAAATCAAGGCTGCCTTGGACAATGCG |
| | | | GGAAAGATTATGAGCCTGACTAAAACCGCCCCCGACT |
| | | | ACCTGGTGGGCCAGCAGCCCGTGGAGGACATTTCCAG |
| | | | CAATCGGATTTATAAAATTTTGGAACTAAACGGGTACG |
| | | | ATCCCCAATATGCGGCTTCCGTCTTTCTGGGATGGGC |
| | | | CACGAAAAAGTTCGGCAAGAGGAACACCATCTGGCTG |
| | | | TTTGGGCCTGCAACTACCGGGAAGACCAACATCGCGG |
| | | | AGGCCATAGCCCACACTGTGCCCTTCTACGGGTGCGT |
| | | | AAACTGGACCAATGAGAACTTTCCCTTCAACGACTGTG |
| | | | TCGACAAGATGGTGATCTGGTGGGAGGAGGGGAAGAT |
| | | | GACCGCCAAGGTCGTGGAGTCGGCCAAAGCCATTCTC |
| | | | GGAGGAAGCAAGGTGCGCGTGGACCAGAAATGCAAG |
| | | | TCCTCGGCCCAGATAGACCCGACTCCCGTGATCGTCA |
| | | | CCTCCAACACCAACATGTGCGCCGTGATTGACGGGAA |
| | | | CTCAACGACCTTCGAACACCAGCAGCCGTTGCAAGAC |
| | | | CGGATGTTCAAATTTGAACTCACCCGCCGTCTGGATCA |
| | | | TGACTTTGGGAAGGTCACCAAGCAGGAAGTCAAAGAC |
| | | | TTTTTCCGGTGGGCAAAGGATCACGTGGTTGAGGTGG |
| | | | AGCATGAATTCTACGTCAAAAAGGGTGGAGCCAAGAAA |
| | | | AGACCCGCCCCCAGTGACGCAGATATAAGTGAGCCCA |
| | | | AACGGGTGCGCGAGTCAGTTGCGCAGCCATCGACGTC |
| | | | AGACGCGGAAGCTTCGATCAACTACGCAGACAGGTAC |
| | | | CAAAACAAATGTTCTCGTCACGTGGGCATGAATCTGAT |
| | | | GCTGTTTCCCTGCAGACAATGCGAGAGAATGAATCAGA |
| | | | ATTCAAATATCTGCTTCACTCACGGACAGAAAGACTGT |
| | | | TTAGAGTGCTTTCCCGTGTCAGAATCTCAACCCGTTTC |
| | | | TGTCGTCAAAAAGGCGTATCAGAAACTGTGCTACATTC |
| | | | ATCATATCATGGGAAAGGTGCCAGACGCTTGCACTGC |
| | | | CTGCGATCTGGTCAATGTGGATTTGGATGACTGCATCT |
| | | | TTGAACAATAAATGATTTAAATCAGGTatgtcttttgttgatcac |
| | | | cctccagattggttggaagaagttggtgaaggtcttcgcgagtttttgggccttgaagc |
| | | | gggcccaccgaaaccaaaacccaatcagcagcatcaagatcaagcccgtggtcttg |
| | | | tgctgcctggttataactatctcggacccggaaacggtctcgatcgaggagagcctgt |
| | | | caacagggcagacgaggtcgcgcgagagcacgacatctcgtacaacgagcagctt |
| | | | gaggcgggagacaacccctacctcaagtacaaccacgcggacgccgagtttcagg |
| | | | agaagctcgccgacgacacatccttcgggggaaacctcggaaaggcagtctttcag |
| | | | gccaagaaaagggttctcgaaccttttggcctgcttgaagagggtcctaagacggcc |
| | | | cctaccggaaagcggatagacgaccactttccaaaaagaaagaaggctcggaccg |
| | | | aagaggactccaagccttccacctcgtcagacgccgaagctggacccagcggatc |
| | | | ccagcagctgcaaatcccagcccaaccagcctcaagtttgggagctgatacaatgtc |
| | | | tgcgggaggtggcggcccattgggcgacaataaccaaggtgccgatggagtggg |
| | | | caatgcctcgggagattggcattgcgattccacgtggatgggggacagagtcgtca |
| | | | ccaagtccacccgaacctgggtgctgcccagctacaacaaccaccagtaccgaga |
| | | | gatcaaaagcggctccgtcgacggaagcaacgccaacgcctactttggatacagca |
| | | | ccccctgggggtactttgactttaaccgcttccacagccactggagcccccgagact |
| | | | ggcaaagactcatcaacaactactggggcttcagaccccggtccctcagagtcaaa |
| | | | atcttcaacattcaagtcaaagaggtcacggtgcaggactccaccaccaccatcgcc |
| | | | aacaacctcacctccaccgtccaagtgtttacggacgacgactaccagctgccctac |
| | | | gtcgtcggcaacgggaccgagcgatccctgccggccttccctccgcaggtctttac |
| | | | gctgccgcagtacgcttacgcgacgctgaaccgcgacaacacagaaaatcccacc |
| | | | gagaggagcagcttcttctgcctagagtactttcccagcaagatgctgagaacgggc |
| | | | aacaactttgagtttacctacaactttgaggaggtgcccttccactccagcttcgctccc |
| | | | agtcagaacctgttcaagctggccaacccgctggtggaccagtacttgtaccgcttc |
| | | | gtgagcacaaataacactggcggagtccagttcaacaagaacctggccgggagat |
| | | | acgccaacacctacaaaaactggttcccggggcccatgggccgaacccagggctg |
| | | | gaacctgggctccggggtcaaccgcgccagtgtcagcgccttcgccacgaccaat |
| | | | aggatggagctcgagggcgcgagttaccaggtgcccccgcagccgaacggcatg |
| | | | accaacaacctccagggcagcaacacctatgccctggagaacactatgatcttcaac |
| | | | agccagccggcgaacccgggcaccaccgccacgtacctcgagggcaacatgctc |
| | | | atcaccagcgagagcgagacgcagccggtgaaccgcgtggcgtacaacgtcggc |
| | | | gggcagatggccaccaacaaccagagctccaccactgcccccgcgaccggcacg |
| | | | tacaacctccaggaaatcgtgcccggcagcgtgtggatggagagggacgtgtacct |
| | | | ccaaggacccatctgggccaagatcccagagacgggggcgcactttcacccctctc |
| | | | cggccatgggcggattcggactcaaacacccaccgcccatgatgctcatcaagaac |
| | | | acgcctgtgcccggaaatatcaccagcttctcggacgtgcccgtcagcagcttcatc |
| | | | acccagtacagcaccgggcaggtcaccgtggagatggagtgggagctcaagaag |
| | | | gaaaactccaagaggtggaacccagagatccagtacacaaacaactacaacgacc |
| | | | cccagtttgtggactttgccccggacagcaccggggaatacagaaccaccagacct |
| | | | atcggaacccgataccttacccgacccctttaaTTGCTTGTTAATCAA |
| | | | TAAACCGTTTAATTCGTTTCAGTTGAACTTTGGTC |
| | | | TCTGCCCTAGGGGAGGTCCTGTATTAGAGGTCAC |
| | | | GTGAGTGTTTTGCGACATTTTGCGACACCATGT |
| | | | GGTCACGCTGGGTATTTAAGCCCGAGTGAGCAC |
| | | | GCAGGGTCTCCATTTTGAAGCGGGAGGTTTGAA |
| | | | GTCTTACCCCCAATAAGGGGATTTGCTCTATTTAAT |
| | | | TAGGAATAAGGTCGATTACTGATAGAACAAATCCA |
| | | | GGCTACTGTGTTTAGTAATCAGATTTGTTCGTGACC |
| | | | GATATGCACGGGCAAAACGGCAGGAGGTTGTTAGC |
| | | | GCGACCTCCTGCCACCCGCTTTCACGAAGGTCATGT |
| | | | GTAAAAGGCCGCAGCGTAACTATTACTAATGAATT |
| | | | CAGGACAGACAGTGGCTACGGCTCAGTTTGGGTTG |
| | | | TGCTGTTGCTGGGCGGCGATGACGCCTGTACGCAT |
| | | | TTGGTGATCCGGTTCTGgaggtcctgtattagaggtcacgtgagtg |
| | | | ttttgcgacattttgcgacaccatgtggtcacgctgggtatttaagcccgagtga |
| | | | gcacgcagggtctccattttgaagcgggaggtttgaaATCCCAATGGC |
| | | | GCGCCGAGCTTGGCTCGAGCATGGTCATAGCTGTT |
| | | | TCCTGTGTGAAATTGTTATCCGCTCACAATTCCACA |
| | | | CAACATACGAGCCGGAAGCATAAAGTGTAAAGCCT |
| | | | GGGGTGCCTAATGAGTGAGCTAACTCACATTAATT |
| | | | GCGTTGCGCTCACTGCCCGCTTTCCAGTCGGGAAA |
| | | | CCTGTCGTGCCAGCTGCATTAATGAATCGGCCAAC |
| | | | GCGCGGGGAGAGGCGGTTTGCGTATTGGGCGCTCT |
| | | | TCCGCTTCCTCGCTCACTGACTCGCTGCGCTCGGTC |
| | | | GTTCGGCTGCGGCGAGCGGTATCAGCTCACTCAAA |
| | | | GGCGGTAATACGGTTATCCACAGAATCAGGGGATA |
| | | | ACGCAGGAAAGAACATGTGAGCAAAAGGCCAGCA |
| | | | AAAGGCCAGGAACCGTAAAAAGGCCGCGTTGCTG |
| | | | GCGTTTTTCCATAGGCTCCGCCCCCCTGACGAGCAT |
| | | | CACAAAAATCGACGCTCAAGTCAGAGGTGGCGAA |
| | | | ACCCGACAGGACTATAAAGATACCAGGCGTTTCCC |
| | | | CCTGGAAGCTCCCTCGTGCGCTCTCCTGTTCCGACC |
| | | | CTGCCGCTTACCGGATACCTGTCCGCCTTTCTCCCT |
| | | | TCGGGAAGCGTGGCGCTTTCTCATAGCTCACGCTGT |
| | | | AGGTATCTCAGTTCGGTGTAGGTCGTTCGCTCCAAG |
| | | | CTGGGCTGTGTGCACGAACCCCCCGTTCAGCCCGA |
| | | | CCGCTGCGCCTTATCCGGTAACTATCGTCTTGAGTC |
| | | | CAACCCGGTAAGACACGACTTATCGCCACTGGCAG |
| | | | CAGCCACTGGTAACAGGATTAGCAGAGCGAGGTAT |
| | | | GTAGGCGGTGCTACAGAGTTCTTGAAGTGGTGGCC |
| | | | TAACTACGGCTACACTAGAAGAACAGTATTTGGTA |
| | | | TCTGCGCTCTGCTGAAGCCAGTTACCTTCGGAAAA |
| | | | AGAGTTGGTAGCTCTTGATCCGGCAAACAAACCAC |
| | | | CGCTGGTAGCGGTGGTTTTTTTGTTTGCAAGCAGCA |
| | | | GATTACGCGCAGAAAAAAAGGATCTCAAGAAGAT |
| | | | CCTTTGATCTTTTCTACGGGGTCTGACGCTCAGTGG |
| | | | AACGAAAACTCACGTTAAGGGATTTTGGTCATGAG |
| | | | ATTATCAAAAAGGATCTTCACCTAGATCCTTTTAAA |
| | | | TTAAAAATGAAGTTTTAAATCAATCTAAAGTATAT |
| | | | ATGAGTAAACTTGGTCTGACAGTTAGAAAAACTCA |
| | | | TCGAGCATCAAATGAAACTGCAATTTATTCATATC |
| | | | AGGATTATCAATACCATATTTTTGAAAAAGCCGTTT |
| | | | CTGTAATGAAGGAGAAAACTCACCGAGGCAGTTCC |
| | | | ATAGGATGGCAAGATCCTGGTATCGGTCTGCGATT |
| | | | CCGACTCGTCCAACATCAATACAACCTATTAATTTC |
| | | | CCCTCGTCAAAAATAAGGTTATCAAGTGAGAAATC |
| | | | ACCATGAGTGACGACTGAATCCGGTGAGAATGGCA |
| | | | AAAGTTTATGCATTTCTTTCCAGACTTGTTCAACAG |
| | | | GCCAGCCATTACGCTCGTCATCAAAATCACTCGCA |
| | | | TCAACCAAACCGTTATTCATTCGTGATTGCGCCTGA |
| | | | GCGAGACGAAATACGCGATCGCTGTTAAAAGGACA |
| | | | ATTACAAACAGGAATCGAATGCAACCGGCGCAGG |
| | | | AACACTGCCAGCGCATCAACAATATTTTCACCTGA |
| | | | ATCAGGATATTCTTCTAATACCTGGAATGCTGTTTT |
| | | | CCCAGGGATCGCAGTGGTGAGTAACCATGCATCAT |
| | | | CAGGAGTACGGATAAAATGCTTGATGGTCGGAAGA |
| | | | GGCATAAATTCCGTCAGCCAGTTTAGTCTGACCATC |
| | | | TCATCTGTAACATCATTGGCAACGCTACCTTTGCCA |
| | | | TGTTTCAGAAACAACTCTGGCGCATCGGGCTTCCC |
| | | | ATACAATCGATAGATTGTCGCACCTGATTGCCCGA |
| | | | CATTATCGCGAGCCCATTTATACCCATATAAATCAG |
| | | | CATCCATGTTGGAATTTAATCGCGGCCTAGAGCAA |
| | | | GACGTTTCCCGTTGAATATGGCTCATACTCTTCCTT |
| | | | TTTCAATATTATTGAAGCATTTATCAGGGTTATTGT |
| | | | CTCATGAGCGGATACATATTTGAATGTATTTAGAA |
| | | | AAATAAACAAATAGGGGTTCCGCGCACATTTCCCC |
| | | | GAAAAGTGCCACCTGACGTCTAAGAAACCATTATT |
| | | | ATCATGACATTAACCTATAAAAATAGGCGTATCAC |
| | | | GAGGCCCTTTCGTC |
| 59 | pYZZ438 | Rep2Cap5-p5.p5 | TCGCGCGTTTCGGTGATGACGGTGAAAACCTCTGA |
| | | | CACATGCAGCTCCCGGAGACGGTCACAGCTTGTCT |
| | | | GTAAGCGGATGCCGGGAGCAGACAAGCCCGTCAG |
| | | | GGCGCGTCAGCGGGTGTTGGCGGGTGTCGGGGCTG |
| | | | GCTTAACTATGCGGCATCAGAGCAGATTGTACTGA |
| | | | GAGTGCACCATATGCGGTGTGAAATACCGCACAGA |
| | | | TGCGTAAGGAGAAAATACCGCATCAGGCGCCATTC |
| | | | GCCATTCAGGCTGCGCAACTGTTGGGAAGGGCGAT |
| | | | CGGTGCGGGCCTCTTCGCTATTACGCCAGCTGGCG |
| | | | AAAGGGGGATGTGCTGCAAGGCGATTAAGTTGGGT |
| | | | AACGCCAGGGTTTTCCCAGTCACGACGTTGTAAAA |
| | | | CGACGGCCAGTGAATTGACGCGTATTGGGATCGCG |
| | | | CAGCCGCCATGCCGGGGTTTTACGAGATTGTGATTAA |
| | | | GGTCCCCAGCGACCTTGACGAGCATCTGCCCGGCATT |
| | | | TCTGACAGCTTTGTGAACTGGGTGGCCGAGAAGGAAT |
| | | | GGGAGTTGCCGCCAGATTCTGACATGGATCTGAATCT |
| | | | GATTGAGCAGGCACCCCTGACCGTGGCCGAGAAGCT |
| | | | GCAGCGCGACTTTCTGACGGAATGGCGCCGTGTGAGT |
| | | | AAGGCCCCGGAGGCTCTTTTCTTTGTGCAATTTGAGAA |
| | | | GGGAGAGAGCTACTTCCACATGCACGTGCTCGTGGAA |
| | | | ACCACCGGGGTGAAATCCATGGTTTTGGGACGTTTCCT |
| | | | GAGTCAGATTCGCGAAAAACTGATTCAGAGAATTTACC |
| | | | GCGGGATCGAGCCGACTTTGCCAAACTGGTTCGCGGT |
| | | | CACAAAGACCAGAAATGGCGCCGGAGGCGGGAACAA |
| | | | GGTGGTGGATGAGTGCTACATCCCCAATTACTTGCTCC |
| | | | CCAAAACCCAGCCTGAGCTCCAGTGGGCGTGGACTAA |
| | | | TATGGAACAGTATTTAAGCGCCTGTTTGAATCTCACGG |
| | | | AGCGTAAACGGTTGGTGGCGCAGCATCTGACGCACGT |
| | | | GTCGCAGACGCAGGAGCAGAACAAAGAGAATCAGAAT |
| | | | CCCAATTCTGATGCGCCGGTGATCAGATCAAAAACTTC |
| | | | AGCCAGGTACATGGAGCTGGTCGGGTGGCTCGTGGA |
| | | | CAAGGGGATTACCTCGGAGAAGCAGTGGATCCAGGAG |
| | | | GACCAGGCCTCATACATCTCCTTCAATGCGGCCTCCAA |
| | | | CTCGCGGTCCCAAATCAAGGCTGCCTTGGACAATGCG |
| | | | GGAAAGATTATGAGCCTGACTAAAACCGCCCCCGACT |
| | | | ACCTGGTGGGCCAGCAGCCCGTGGAGGACATTTCCAG |
| | | | CAATCGGATTTATAAAATTTTGGAACTAAACGGGTACG |
| | | | ATCCCCAATATGCGGCTTCCGTCTTTCTGGGATGGGC |
| | | | CACGAAAAAGTTCGGCAAGAGGAACACCATCTGGCTG |
| | | | TTTGGGCCTGCAACTACCGGGAAGACCAACATCGCGG |
| | | | AGGCCATAGCCCACACTGTGCCCTTCTACGGGTGCGT |
| | | | AAACTGGACCAATGAGAACTTTCCCTTCAACGACTGTG |
| | | | TCGACAAGATGGTGATCTGGTGGGAGGAGGGGAAGAT |
| | | | GACCGCCAAGGTCGTGGAGTCGGCCAAAGCCATTCTC |
| | | | GGAGGAAGCAAGGTGCGCGTGGACCAGAAATGCAAG |
| | | | TCCTCGGCCCAGATAGACCCGACTCCCGTGATCGTCA |
| | | | CCTCCAACACCAACATGTGCGCCGTGATTGACGGGAA |
| | | | CTCAACGACCTTCGAACACCAGCAGCCGTTGCAAGAC |
| | | | CGGATGTTCAAATTTGAACTCACCCGCCGTCTGGATCA |
| | | | TGACTTTGGGAAGGTCACCAAGCAGGAAGTCAAAGAC |
| | | | TTTTTCCGGTGGGCAAAGGATCACGTGGTTGAGGTGG |
| | | | AGCATGAATTCTACGTCAAAAAGGGTGGAGCCAAGAAA |
| | | | AGACCCGCCCCCAGTGACGCAGATATAAGTGAGCCCA |
| | | | AACGGGTGCGCGAGTCAGTTGCGCAGCCATCGACGTC |
| | | | AGACGCGGAAGCTTCGATCAACTACGCAGACAGGTAC |
| | | | CAAAACAAATGTTCTCGTCACGTGGGCATGAATCTGAT |
| | | | GCTGTTTCCCTGCAGACAATGCGAGAGAATGAATCAGA |
| | | | ATTCAAATATCTGCTTCACTCACGGACAGAAAGACTGT |
| | | | TTAGAGTGCTTTCCCGTGTCAGAATCTCAACCCGTTTC |
| | | | TGTCGTCAAAAAGGCGTATCAGAAACTGTGCTACATTC |
| | | | ATCATATCATGGGAAAGGTGCCAGACGCTTGCACTGC |
| | | | CTGCGATCTGGTCAATGTGGATTTGGATGACTGCATCT |
| | | | TTGAACAATAAATGATTTAAATCAGGTatgtcttttgttgatcac |
| | | | cctccagattggttggaagaagttggtgaaggtcttcgcgagtttttgggccttgaagc |
| | | | gggcccaccgaaaccaaaacccaatcagcagcatcaagatcaagcccgtggtcttg |
| | | | tgctgcctggttataactatctcggacccggaaacggtctcgatcgaggagagcctgt |
| | | | caacagggcagacgaggtcgcgcgagagcacgacatctcgtacaacgagcagctt |
| | | | gaggcgggagacaacccctacctcaagtacaaccacgcggacgccgagtttcagg |
| | | | agaagctcgccgacgacacatccttcgggggaaacctcggaaaggcagtctttcag |
| | | | gccaagaaaagggttctcgaaccttttggcctggttgaagagggtgctaagacggcc |
| | | | cctaccggaaagcggatagacgaccactttccaaaaagaaagaaggctcggaccg |
| | | | aagaggactccaagccttccacctcgtcagacgccgaagctggacccagcggatc |
| | | | ccagcagctgcaaatcccagcccaaccagcctcaagtttgggagctgatacaatgtc |
| | | | tgcgggaggtggcggcccattgggcgacaataaccaaggtgccgatggagtggg |
| | | | caatgcctcgggagattggcattgcgattccacgtggatgggggacagagtcgtca |
| | | | ccaagtccacccgaacctgggtgctgcccagctacaacaaccaccagtaccgaga |
| | | | gatcaaaagcggctccgtcgacggaagcaacgccaacgcctactttggatacagca |
| | | | ccccctgggggtactttgactttaaccgcttccacagccactggagcccccgagact |
| | | | ggcaaagactcatcaacaactactggggcttcagaccccggtccctcagagtcaaa |
| | | | atcttcaacattcaagtcaaagaggtcacggtgcaggactccaccaccaccatcgcc |
| | | | aacaacctcacctccaccgtccaagtgtttacggacgacgactaccagctgccctac |
| | | | gtcgtcggcaacgggaccgagggatgcctgccggccttccctccgcaggtctttac |
| | | | gctgccgcagtacggttacgcgacgctgaaccgcgacaacacagaaaatcccacc |
| | | | gagaggagcagcttcttctgcctagagtactttcccagcaagatgctgagaacgggc |
| | | | aacaactttgagtttacctacaactttgaggaggtgcccttccactccagcttcgctccc |
| | | | agtcagaacctgttcaagctggccaacccgctggtggaccagtacttgtaccgcttc |
| | | | gtgagcacaaataacactggcggagtccagttcaacaagaacctggccgggagat |
| | | | acgccaacacctacaaaaactggttcccggggcccatgggccgaacccagggctg |
| | | | gaacctgggctccggggtcaaccgcgccagtgtcagcgccttcgccacgaccaat |
| | | | aggatggagctcgagggcgcgagttaccaggtgcccccgcagccgaacggcatg |
| | | | accaacaacctccagggcagcaacacctatgccctggagaacactatgatcttcaac |
| | | | agccagccggcgaacccgggcaccaccgccacgtacctcgagggcaacatgctc |
| | | | atcaccagcgagagcgagacgcagccggtgaaccgcgtggcgtacaacgtcggc |
| | | | gggcagatggccaccaacaaccagagctccaccactgcccccgcgaccggcacg |
| | | | tacaacctccaggaaatcgtgcccggcagcgtgtggatggagagggacgtgtacct |
| | | | ccaaggacccatctgggccaagatcccagagacgggggcgcactttcacccctctc |
| | | | cggccatgggcggattcggactcaaacacccaccgcccatgatgctcatcaagaac |
| | | | acgcctgtgcccggaaatatcaccagcttctcggacgtgcccgtcagcagcttcatc |
| | | | acccagtacagcaccgggcaggtcaccgtggagatggagtgggagctcaagaag |
| | | | gaaaactccaagaggtggaacccagagatccagtacacaaacaactacaacgacc |
| | | | cccagtttgtggactttgccccggacagcaccggggaatacagaaccaccagacct |
| | | | atcggaacccgataccttacccgacccctttaaTTGCTTGTTAATCAA |
| | | | TAAACCGTTTAATTCGTTTCAGTTGAACTTTGGTC |
| | | | TCTGCCCTAGGGGAGGTCCTGTATTAGAGGTCAC |
| | | | GTGAGTGTTTTGCGACATTTTGCGACACCATGT |
| | | | GGTCACGCTGGGTATTTAAGCCCGAGTGAGCAC |
| | | | GCAGGGTCTCCATTTTGAAGCGGGAGGTTTGAA |
| | | | CATACAGACGAAACTAGTTGGAGATTATCGAGCAT |
| | | | TCTATCACGTCGACGACCACTAGTGAGTTACTAGA |
| | | | GCCGAGGGGTAACGTTGATGCTCCTAAAAAgaggtcct |
| | | | gtattagaggtcacgtgagtgttttgcgacattttgcgacaccatgtggtcacgct |
| | | | gggtatttaagcccgagtgagcacgcagggtctccattttgaagcgggaggttt |
| | | | gaaATCCCAATGGCGCGCCGAGCTTGGCTCGAGCAT |
| | | | GGTCATAGCTGTTTCCTGTGTGAAATTGTTATCCGC |
| | | | TCACAATTCCACACAACATACGAGCCGGAAGCATA |
| | | | AAGTGTAAAGCCTGGGGTGCCTAATGAGTGAGCTA |
| | | | ACTCACATTAATTGCGTTGCGCTCACTGCCCGCTTT |
| | | | CCAGTCGGGAAACCTGTCGTGCCAGCTGCATTAAT |
| | | | GAATCGGCCAACGCGCGGGGAGAGGCGGTTTGCGT |
| | | | ATTGGGCGCTCTTCCGCTTCCTCGCTCACTGACTCG |
| | | | CTGCGCTCGGTCGTTCGGCTGCGGCGAGCGGTATC |
| | | | AGCTCACTCAAAGGCGGTAATACGGTTATCCACAG |
| | | | AATCAGGGGATAACGCAGGAAAGAACATGTGAGC |
| | | | AAAAGGCCAGCAAAAGGCCAGGAACCGTAAAAAG |
| | | | GCCGCGTTGCTGGCGTTTTTCCATAGGCTCCGCCCC |
| | | | CCTGACGAGCATCACAAAAATCGACGCTCAAGTCA |
| | | | GAGGTGGCGAAACCCGACAGGACTATAAAGATAC |
| | | | CAGGCGTTTCCCCCTGGAAGCTCCCTCGTGCGCTCT |
| | | | CCTGTTCCGACCCTGCCGCTTACCGGATACCTGTCC |
| | | | GCCTTTCTCCCTTCGGGAAGCGTGGCGCTTTCTCAT |
| | | | AGCTCACGCTGTAGGTATCTCAGTTCGGTGTAGGTC |
| | | | GTTCGCTCCAAGCTGGGCTGTGTGCACGAACCCCC |
| | | | CGTTCAGCCCGACCGCTGCGCCTTATCCGGTAACTA |
| | | | TCGTCTTGAGTCCAACCCGGTAAGACACGACTTAT |
| | | | CGCCACTGGCAGCAGCCACTGGTAACAGGATTAGC |
| | | | AGAGCGAGGTATGTAGGCGGTGCTACAGAGTTCTT |
| | | | GAAGTGGTGGCCTAACTACGGCTACACTAGAAGAA |
| | | | CAGTATTTGGTATCTGCGCTCTGCTGAAGCCAGTTA |
| | | | CCTTCGGAAAAAGAGTTGGTAGCTCTTGATCCGGC |
| | | | AAACAAACCACCGCTGGTAGCGGTGGTTTTTTTGTT |
| | | | TGCAAGCAGCAGATTACGCGCAGAAAAAAAGGAT |
| | | | CTCAAGAAGATCCTTTGATCTTTTCTACGGGGTCTG |
| | | | ACGCTCAGTGGAACGAAAACTCACGTTAAGGGATT |
| | | | TTGGTCATGAGATTATCAAAAAGGATCTTCACCTA |
| | | | GATCCTTTTAAATTAAAAATGAAGTTTTAAATCAAT |
| | | | CTAAAGTATATATGAGTAAACTTGGTCTGACAGTT |
| | | | AGAAAAACTCATCGAGCATCAAATGAAACTGCAAT |
| | | | TTATTCATATCAGGATTATCAATACCATATTTTTGA |
| | | | AAAAGCCGTTTCTGTAATGAAGGAGAAAACTCACC |
| | | | GAGGCAGTTCCATAGGATGGCAAGATCCTGGTATC |
| | | | GGTCTGCGATTCCGACTCGTCCAACATCAATACAA |
| | | | CCTATTAATTTCCCCTCGTCAAAAATAAGGTTATCA |
| | | | AGTGAGAAATCACCATGAGTGACGACTGAATCCGG |
| | | | TGAGAATGGCAAAAGTTTATGCATTTCTTTCCAGAC |
| | | | TTGTTCAACAGGCCAGCCATTACGCTCGTCATCAA |
| | | | AATCACTCGCATCAACCAAACCGTTATTCATTCGTG |
| | | | ATTGCGCCTGAGCGAGACGAAATACGCGATCGCTG |
| | | | TTAAAAGGACAATTACAAACAGGAATCGAATGCAA |
| | | | CCGGCGCAGGAACACTGCCAGCGCATCAACAATAT |
| | | | TTTCACCTGAATCAGGATATTCTTCTAATACCTGGA |
| | | | ATGCTGTTTTCCCAGGGATCGCAGTGGTGAGTAAC |
| | | | CATGCATCATCAGGAGTACGGATAAAATGCTTGAT |
| | | | GGTCGGAAGAGGCATAAATTCCGTCAGCCAGTTTA |
| | | | GTCTGACCATCTCATCTGTAACATCATTGGCAACGC |
| | | | TACCTTTGCCATGTTTCAGAAACAACTCTGGCGCAT |
| | | | CGGGCTTCCCATACAATCGATAGATTGTCGCACCT |
| | | | GATTGCCCGACATTATCGCGAGCCCATTTATACCCA |
| | | | TATAAATCAGCATCCATGTTGGAATTTAATCGCGG |
| | | | CCTAGAGCAAGACGTTTCCCGTTGAATATGGCTCA |
| | | | TACTCTTCCTTTTTCAATATTATTGAAGCATTTATCA |
| | | | GGGTTATTGTCTCATGAGCGGATACATATTTGAATG |
| | | | TATTTAGAAAAATAAACAAATAGGGGTTCCGCGCA |
| | | | CATTTCCCCGAAAAGTGCCACCTGACGTCTAAGAA |
| | | | ACCATTATTATCATGACATTAACCTATAAAAATAG |
| | | | GCGTATCACGAGGCCCTTTCGTC |
| 60 | pYZZ439 | Rep2Cap5-p5. | TCGCGCGTTTCGGTGATGACGGTGAAAACCTCTGACACAT |
| | | p5 | GCAGCTCCCGGAGACGGTCACAGCTTGTCTGTAAGCGGAT |
| | | | GCCGGGAGCAGACAAGCCCGTCAGGGCGCGTCAGCGGGTG |
| | | | TTGGCGGGTGTCGGGGCTGGCTTAACTATGCGGCATCAGA |
| | | | GCAGATTGTACTGAGAGTGCACCATATGCGGTGTGAAATA |
| | | | CCGCACAGATGCGTAAGGAGAAAATACCGCATCAGGCGCC |
| | | | ATTCGCCATTCAGGCTGCGCAACTGTTGGGAAGGGCGATC |
| | | | GGTGCGGGCCTCTTCGCTATTACGCCAGCTGGCGAAAGGG |
| | | | GGATGTGCTGCAAGGCGATTAAGTTGGGTAACGCCAGGGT |
| | | | TTTCCCAGTCACGACGTTGTAAAACGACGGCCAGTGAATT |
| | | | GACGCGTATTGGGATCGCGCAGCCGCCATGCCGGGGTTTT |
| | | | ACGAGATTGTGATTAAGGTCCCCAGCGACCTTGACGAGCA |
| | | | TCTGCCCGGCATTTCTGACAGCTTTGTGAACTGGGTGGCCG |
| | | | AGAAGGAATGGGAGTTGCCGCCAGATTCTGACATGGATCT |
| | | | GAATCTGATTGAGCAGGCACCCCTGACCGTGGCCGAGAAG |
| | | | CTGCAGCGCGACTTTCTGACGGAATGGCGCCGTGTGAGTA |
| | | | AGGCCCCGGAGGCTCTTTTCTTTGTGCAATTTGAGAAGGG |
| | | | AGAGAGCTACTTCCACATGCACGTGCTCGTGGAAACCACC |
| | | | GGGGTGAAATCCATGGTTTTGGGACGTTTCCTGAGTCAGA |
| | | | TTCGCGAAAAACTGATTCAGAGAATTTACCGCGGGATCGA |
| | | | GCCGACTTTGCCAAACTGGTTCGCGGTCACAAAGACCAGA |
| | | | AATGGCGCCGGAGGCGGGAACAAGGTGGTGGATGAGTGCT |
| | | | ACATCCCCAATTACTTGCTCCCCAAAACCCAGCCTGAGCTC |
| | | | CAGTGGGCGTGGACTAATATGGAACAGTATTTAAGCGCCT |
| | | | GTTTGAATCTCACGGAGCGTAAACGGTTGGTGGCGCAGCA |
| | | | TCTGACGCACGTGTCGCAGACGCAGGAGCAGAACAAAGAG |
| | | | AATCAGAATCCCAATTCTGATGCGCCGGTGATCAGATCAA |
| | | | AAACTTCAGCCAGGTACATGGAGCTGGTCGGGTGGCTCGT |
| | | | GGACAAGGGGATTACCTCGGAGAAGCAGTGGATCCAGGAG |
| | | | GACCAGGCCTCATACATCTCCTTCAATGCGGCCTCCAACTC |
| | | | GCGGTCCCAAATCAAGGCTGCCTTGGACAATGCGGGAAAG |
| | | | ATTATGAGCCTGACTAAAACCGCCCCCGACTACCTGGTGG |
| | | | GCCAGCAGCCCGTGGAGGACATTTCCAGCAATCGGATTTA |
| | | | TAAAATTTTGGAACTAAACGGGTACGATCCCCAATATGCG |
| | | | GCTTCCGTCTTTCTGGGATGGGCCACGAAAAAGTTCGGCA |
| | | | AGAGGAACACCATCTGGCTGTTTGGGCCTGCAACTACCGG |
| | | | GAAGACCAACATCGCGGAGGCCATAGCCCACACTGTGCCCT |
| | | | TCTACGGGTGCGTAAACTGGACCAATGAGAACTTTCCCTT |
| | | | CAACGACTGTGTCGACAAGATGGTGATCTGGTGGGAGGAG |
| | | | GGGAAGATGACCGCCAAGGTCGTGGAGTCGGCCAAAGCCA |
| | | | TTCTCGGAGGAAGCAAGGTGCGCGTGGACCAGAAATGCAA |
| | | | GTCCTCGGCCCAGATAGACCCGACTCCCGTGATCGTCACCT |
| | | | CCAACACCAACATGTGCGCCGTGATTGACGGGAACTCAAC |
| | | | GACCTTCGAACACCAGCAGCCGTTGCAAGACCGGATGTTC |
| | | | AAATTTGAACTCACCCGCCGTCTGGATCATGACTTTGGGA |
| | | | AGGTCACCAAGCAGGAAGTCAAAGACTTTTTCCGGTGGGC |
| | | | AAAGGATCACGTGGTTGAGGTGGAGCATGAATTCTACGTC |
| | | | AAAAAGGGTGGAGCCAAGAAAAGACCCGCCCCCAGTGACG |
| | | | CAGATATAAGTGAGCCCAAACGGGTGCGCGAGTCAGTTGC |
| | | | GCAGCCATCGACGTCAGACGCGGAAGCTTCGATCAACTAC |
| | | | GCAGACAGGTACCAAAACAAATGTTCTCGTCACGTGGGCA |
| | | | TGAATCTGATGCTGTTTCCCTGCAGACAATGCGAGAGAAT |
| | | | GAATCAGAATTCAAATATCTGCTTCACTCACGGACAGAAA |
| | | | GACTGTTTAGAGTGCTTTCCCGTGTCAGAATCTCAACCCGT |
| | | | TTCTGTCGTCAAAAAGGCGTATCAGAAACTGTGCTACATT |
| | | | CATCATATCATGGGAAAGGTGCCAGACGCTTGCACTGCCT |
| | | | GCGATCTGGTCAATGTGGATTTGGATGACTGCATCTTTGA |
| | | | ACAATAAATGATTTAAATCAGGTatgtcttttgttgatcaccctccag |
| | | | attggttggaagaagttggtgaaggtcttcgcgagtttttgggccttgaagcggg |
| | | | cccaccgaaaccaaaacccaatcagcagcatcaagatcaagcccgtggtcttgt |
| | | | gctgcctggttataactatctcggacccggaaacggtctcgatcgaggagagcc |
| | | | tgtcaacagggcagacgaggtcgcgcgagagcacgacatctcgtacaacgagc |
| | | | agcttgaggcgggagacaacccctacctcaagtacaaccacgcggacgccgag |
| | | | tttcaggagaagctcgccgacgacacatccttcgggggaaacctcggaaaggc |
| | | | agtctttcaggccaagaaaagggttctcgaaccttttggcctggttgaagagggt |
| | | | gctaagacggcccctaccggaaagcggatagacgaccactttccaaaaagaaa |
| | | | gaaggctcggaccgaagaggactccaagccttccacctcgtcagacgccgaag |
| | | | ctggacccagcggatcccagcagctgcaaatcccagcccaaccagcctcaagtt |
| | | | tgggagctgatacaatgtctgcgggaggtggcggcccattgggcgacaataac |
| | | | caaggtgccgatggagtgggcaatgcctcgggagattggcattgcgattccacg |
| | | | tggatgggggacagagtcgtcaccaagtccacccgaacctgggtgctgcccag |
| | | | ctacaacaaccaccagtaccgagagatcaaaagcggctccgtcgacggaagc |
| | | | aacgccaacgcctactttggatacagcaccccctgggggtactttgactttaacc |
| | | | gcttccacagccactggagcccccgagactggcaaagactcatcaacaactact |
| | | | ggggcttcagaccccggtccctcagagtcaaaatcttcaacattcaagtcaaag |
| | | | aggtcacggtgcaggactccaccaccaccatcgccaacaacctcacctccaccg |
| | | | tccaagtgtttacggacgacgactaccagctgccctacgtcgtcggcaacggga |
| | | | ccgagggatgcctgccggccttccctccgcaggtctttacgctgccgcagtacgg |
| | | | ttacgcgacgctgaaccgcgacaacacagaaaatcccaccgagaggagcagc |
| | | | ttcttctgcctagagtactttcccagcaagatgctgagaacgggcaacaactttg |
| | | | agtttacctacaactttgaggaggtgcccttccactccagcttcgctcccagtcag |
| | | | aacctgttcaagctggccaacccgctggtggaccagtacttgtaccgcttcgtga |
| | | | gcacaaataacactggcggagtccagttcaacaagaacctggccgggagatac |
| | | | gccaacacctacaaaaactggttcccggggcccatgggccgaacccagggctg |
| | | | gaacctgggctccggggtcaaccgcgccagtgtcagcgccttcgccacgacca |
| | | | ataggatggagctcgagggcgcgagttaccaggtgcccccgcagccgaacggc |
| | | | atgaccaacaacctccagggcagcaacacctatgccctggagaacactatgatc |
| | | | ttcaacagccagccggcgaacccgggcaccaccgccacgtacctcgagggcaa |
| | | | catgctcatcaccagcgagagcgagacgcagccggtgaaccgcgtggcgtaca |
| | | | acgtcggcgggcagatggccaccaacaaccagagctccaccactgcccccgcg |
| | | | accggcacgtacaacctccaggaaatcgtgcccggcagcgtgtggatggagag |
| | | | ggacgtgtacctccaaggacccatctgggccaagatcccagagacgggggcgc |
| | | | actttcacccctctccggccatgggcggattcggactcaaacacccaccgcccat |
| | | | gatgctcatcaagaacacgcctgtgcccggaaatatcaccagcttctcggacgt |
| | | | gcccgtcagcagcttcatcacccagtacagcaccgggcaggtcaccgtggagat |
| | | | ggagtgggagctcaagaaggaaaactccaagaggtggaacccagagatccag |
| | | | tacacaaacaactacaacgacccccagtttgtggactttgccccggacagcacc |
| | | | ggggaatacagaaccaccagacctatcggaacccgataccttacccgacccctt |
| | | | taaTTGCTTGTTAATCAATAAACCGTTTAATTCGTTTCAGT |
| | | | TGAACTTTGGTCTCTGCCCTAGGGGAGGTCCTGTATTAGA |
| | | | GGTCACGTGAGTGTTTTGCGACATTTTGCGACACCATGTG |
| | | | GTCACGCTGGGTATTTAAGCCCGAGTGAGCACGCAGGGTC |
| | | | TCCATTTTGAAGCGGGAGGTTTGAACATACAGACGAAACT |
| | | | AGTTGGAGATTATCGAGCATTCTATCACGTCGACGACCAC |
| | | | TAGTGAGTTACTAGAGCCGAGGGGTAACGTTGATGCTCCT |
| | | | AAAAATCTCTCGGTCGATGCAAGCGATTACACTCCTGTCA |
| | | | CATCATAATCGTTTGCTATTCAGGGCTTGATCAACACTGG |
| | | | ATTGCTTTTCACTTAAAGTATTATGgaggtcctgtattagaggtca |
| | | | cgtgagtgttttgcgacattttgcgacaccatgtggtcacgctgggtatttaagcc |
| | | | cgagtgagcacgcagggtctccattttgaagcgggaggtttgaaATCCCAA |
| | | | TGGCGCGCCGAGCTTGGCTCGAGCATGGTCATAGCTGTTTC |
| | | | CTGTGTGAAATTGTTATCCGCTCACAATTCCACACAACAT |
| | | | ACGAGCCGGAAGCATAAAGTGTAAAGCCTGGGGTGCCTAA |
| | | | TGAGTGAGCTAACTCACATTAATTGCGTTGCGCTCACTGC |
| | | | CCGCTTTCCAGTCGGGAAACCTGTCGTGCCAGCTGCATTAA |
| | | | TGAATCGGCCAACGCGCGGGGAGAGGCGGTTTGCGTATTG |
| | | | GGCGCTCTTCCGCTTCCTCGCTCACTGACTCGCTGCGCTCG |
| | | | GTCGTTCGGCTGCGGCGAGCGGTATCAGCTCACTCAAAGG |
| | | | CGGTAATACGGTTATCCACAGAATCAGGGGATAACGCAGG |
| | | | AAAGAACATGTGAGCAAAAGGCCAGCAAAAGGCCAGGAAC |
| | | | CGTAAAAAGGCCGCGTTGCTGGCGTTTTTCCATAGGCTCC |
| | | | GCCCCCCTGACGAGCATCACAAAAATCGACGCTCAAGTCA |
| | | | GAGGTGGCGAAACCCGACAGGACTATAAAGATACCAGGCG |
| | | | TTTCCCCCTGGAAGCTCCCTCGTGCGCTCTCCTGTTCCGAC |
| | | | CCTGCCGCTTACCGGATACCTGTCCGCCTTTCTCCCTTCGG |
| | | | GAAGCGTGGCGCTTTCTCATAGCTCACGCTGTAGGTATCTC |
| | | | AGTTCGGTGTAGGTCGTTCGCTCCAAGCTGGGCTGTGTGC |
| | | | ACGAACCCCCCGTTCAGCCCGACCGCTGCGCCTTATCCGGT |
| | | | AACTATCGTCTTGAGTCCAACCCGGTAAGACACGACTTAT |
| | | | CGCCACTGGCAGCAGCCACTGGTAACAGGATTAGCAGAGC |
| | | | GAGGTATGTAGGCGGTGCTACAGAGTTCTTGAAGTGGTGG |
| | | | CCTAACTACGGCTACACTAGAAGAACAGTATTTGGTATCT |
| | | | GCGCTCTGCTGAAGCCAGTTACCTTCGGAAAAAGAGTTGG |
| | | | TAGCTCTTGATCCGGCAAACAAACCACCGCTGGTAGCGGT |
| | | | GGTTTTTTTGTTTGCAAGCAGCAGATTACGCGCAGAAAAA |
| | | | AAGGATCTCAAGAAGATCCTTTGATCTTTTCTACGGGGTC |
| | | | TGACGCTCAGTGGAACGAAAACTCACGTTAAGGGATTTTG |
| | | | GTCATGAGATTATCAAAAAGGATCTTCACCTAGATCCTTT |
| | | | TAAATTAAAAATGAAGTTTTAAATCAATCTAAAGTATATA |
| | | | TGAGTAAACTTGGTCTGACAGTTAGAAAAACTCATCGAGC |
| | | | ATCAAATGAAACTGCAATTTATTCATATCAGGATTATCAA |
| | | | TACCATATTTTTGAAAAAGCCGTTTCTGTAATGAAGGAGA |
| | | | AAACTCACCGAGGCAGTTCCATAGGATGGCAAGATCCTGG |
| | | | TATCGGTCTGCGATTCCGACTCGTCCAACATCAATACAACC |
| | | | TATTAATTTCCCCTCGTCAAAAATAAGGTTATCAAGTGAG |
| | | | AAATCACCATGAGTGACGACTGAATCCGGTGAGAATGGCA |
| | | | AAAGTTTATGCATTTCTTTCCAGACTTGTTCAACAGGCCA |
| | | | GCCATTACGCTCGTCATCAAAATCACTCGCATCAACCAAAC |
| | | | CGTTATTCATTCGTGATTGCGCCTGAGCGAGACGAAATAC |
| | | | GCGATCGCTGTTAAAAGGACAATTACAAACAGGAATCGAA |
| | | | TGCAACCGGCGCAGGAACACTGCCAGCGCATCAACAATAT |
| | | | TTTCACCTGAATCAGGATATTCTTCTAATACCTGGAATGC |
| | | | TGTTTTCCCAGGGATCGCAGTGGTGAGTAACCATGCATCA |
| | | | TCAGGAGTACGGATAAAATGCTTGATGGTCGGAAGAGGCA |
| | | | TAAATTCCGTCAGCCAGTTTAGTCTGACCATCTCATCTGT |
| | | | AACATCATTGGCAACGCTACCTTTGCCATGTTTCAGAAAC |
| | | | AACTCTGGCGCATCGGGCTTCCCATACAATCGATAGATTG |
| | | | TCGCACCTGATTGCCCGACATTATCGCGAGCCCATTTATAC |
| | | | CCATATAAATCAGCATCCATGTTGGAATTTAATCGCGGCC |
| | | | TAGAGCAAGACGTTTCCCGTTGAATATGGCTCATACTCTT |
| | | | CCTTTTTCAATATTATTGAAGCATTTATCAGGGTTATTGT |
| | | | CTCATGAGCGGATACATATTTGAATGTATTTAGAAAAATA |
| | | | AACAAATAGGGGTTCCGCGCACATTTCCCCGAAAAGTGCC |
| | | | ACCTGACGTCTAAGAAACCATTATTATCATGACATTAACC |
| | | | TATAAAAATAGGCGTATCACGAGGCCCTTTCGTC |
| 61 | pYZZ440 | Rep2Cap5-p5. | TCGCGCGTTTCGGTGATGACGGTGAAAACCTCTGA |
| | | p5 | CACATGCAGCTCCCGGAGACGGTCACAGCTTGTCT |
| | | | GTAAGCGGATGCCGGGAGCAGACAAGCCCGTCAG |
| | | | GGCGCGTCAGCGGGTGTTGGCGGGTGTCGGGGCTG |
| | | | GCTTAACTATGCGGCATCAGAGCAGATTGTACTGA |
| | | | GAGTGCACCATATGCGGTGTGAAATACCGCACAGA |
| | | | TGCGTAAGGAGAAAATACCGCATCAGGCGCCATTC |
| | | | GCCATTCAGGCTGCGCAACTGTTGGGAAGGGCGAT |
| | | | CGGTGCGGGCCTCTTCGCTATTACGCCAGCTGGCG |
| | | | AAAGGGGGATGTGCTGCAAGGCGATTAAGTTGGGT |
| | | | AACGCCAGGGTTTTCCCAGTCACGACGTTGTAAAA |
| | | | CGACGGCCAGTGAATTGACGCGTATTGGGATCGCG |
| | | | CAGCCGCCATGCCGGGGTTTTACGAGATTGTGATTAA |
| | | | GGTCCCCAGCGACCTTGACGAGCATCTGCCCGGCATT |
| | | | TCTGACAGCTTTGTGAACTGGGTGGCCGAGAAGGAAT |
| | | | GGGAGTTGCCGCCAGATTCTGACATGGATCTGAATCT |
| | | | GATTGAGCAGGCACCCCTGACCGTGGCCGAGAAGCT |
| | | | GCAGCGCGACTTTCTGACGGAATGGCGCCGTGTGAGT |
| | | | AAGGCCCCGGAGGCTCTTTTCTTTGTGCAATTTGAGAA |
| | | | GGGAGAGAGCTACTTCCACATGCACGTGCTCGTGGAA |
| | | | ACCACCGGGGTGAAATCCATGGTTTTGGGACGTTTCCT |
| | | | GAGTCAGATTCGCGAAAAACTGATTCAGAGAATTTACC |
| | | | GCGGGATCGAGCCGACTTTGCCAAACTGGTTCGCGGT |
| | | | CACAAAGACCAGAAATGGCGCCGGAGGCGGGAACAA |
| | | | GGTGGTGGATGAGTGCTACATCCCCAATTACTTGCTCC |
| | | | CCAAAACCCAGCCTGAGCTCCAGTGGGCGTGGACTAA |
| | | | TATGGAACAGTATTTAAGCGCCTGTTTGAATCTCACGG |
| | | | AGCGTAAACGGTTGGTGGCGCAGCATCTGACGCACGT |
| | | | GTCGCAGACGCAGGAGCAGAACAAAGAGAATCAGAAT |
| | | | CCCAATTCTGATGCGCCGGTGATCAGATCAAAAACTTC |
| | | | AGCCAGGTACATGGAGCTGGTCGGGTGGCTCGTGGA |
| | | | CAAGGGGATTACCTCGGAGAAGCAGTGGATCCAGGAG |
| | | | GACCAGGCCTCATACATCTCCTTCAATGCGGCCTCCAA |
| | | | CTCGCGGTCCCAAATCAAGGCTGCCTTGGACAATGCG |
| | | | GGAAAGATTATGAGCCTGACTAAAACCGCCCCCGACT |
| | | | ACCTGGTGGGCCAGCAGCCCGTGGAGGACATTTCCAG |
| | | | CAATCGGATTTATAAAATTTTGGAACTAAACGGGTACG |
| | | | ATCCCCAATATGCGGCTTCCGTCTTTCTGGGATGGGC |
| | | | CACGAAAAAGTTCGGCAAGAGGAACACCATCTGGCTG |
| | | | TTTGGGCCTGCAACTACCGGGAAGACCAACATCGCGG |
| | | | AGGCCATAGCCCACACTGTGCCCTTCTACGGGTGCGT |
| | | | AAACTGGACCAATGAGAACTTTCCCTTCAACGACTGTG |
| | | | TCGACAAGATGGTGATCTGGTGGGAGGAGGGGAAGAT |
| | | | GACCGCCAAGGTCGTGGAGTCGGCCAAAGCCATTCTC |
| | | | GGAGGAAGCAAGGTGCGCGTGGACCAGAAATGCAAG |
| | | | TCCTCGGCCCAGATAGACCCGACTCCCGTGATCGTCA |
| | | | CCTCCAACACCAACATGTGCGCCGTGATTGACGGGAA |
| | | | CTCAACGACCTTCGAACACCAGCAGCCGTTGCAAGAC |
| | | | CGGATGTTCAAATTTGAACTCACCCGCCGTCTGGATCA |
| | | | TGACTTTGGGAAGGTCACCAAGCAGGAAGTCAAAGAC |
| | | | TTTTTCCGGTGGGCAAAGGATCACGTGGTTGAGGTGG |
| | | | AGCATGAATTCTACGTCAAAAAGGGTGGAGCCAAGAAA |
| | | | AGACCCGCCCCCAGTGACGCAGATATAAGTGAGCCCA |
| | | | AACGGGTGCGCGAGTCAGTTGCGCAGCCATCGACGTC |
| | | | AGACGCGGAAGCTTCGATCAACTACGCAGACAGGTAC |
| | | | CAAAACAAATGTTCTCGTCACGTGGGCATGAATCTGAT |
| | | | GCTGTTTCCCTGCAGACAATGCGAGAGAATGAATCAGA |
| | | | ATTCAAATATCTGCTTCACTCACGGACAGAAAGACTGT |
| | | | TTAGAGTGCTTTCCCGTGTCAGAATCTCAACCCGTTTC |
| | | | TGTCGTCAAAAAGGCGTATCAGAAACTGTGCTACATTC |
| | | | ATCATATCATGGGAAAGGTGCCAGACGCTTGCACTGC |
| | | | CTGCGATCTGGTCAATGTGGATTTGGATGACTGCATCT |
| | | | TTGAACAATAAATGATTTAAATCAGGTatgtcttttgttgatcac |
| | | | cctccagattggttggaagaagttggtgaaggtcttcgcgagtttttgggccttgaagc |
| | | | gggcccaccgaaaccaaaacccaatcagcagcatcaagatcaagcccgtggtcttg |
| | | | tgctgcctggttataactatctcggacccggaaacggtctcgatcgaggagagcctgt |
| | | | caacagggcagacgaggtcgcgcgagagcacgacatctcgtacaacgagcagctt |
| | | | gaggcgggagacaacccctacctcaagtacaaccacgcggacgccgagtttcagg |
| | | | agaagctcgccgacgacacatccttcgggggaaacctcggaaaggcagtctttcag |
| | | | gccaagaaaagggttctcgaaccttttggcctggttgaagagggtgctaagacggcc |
| | | | cctaccggaaagcggatagacgaccactttccaaaaagaaagaaggctcggaccg |
| | | | aagaggactccaagccttccacctcgtcagacgccgaagctggacccagcggatc |
| | | | ccagcagctgcaaatcccagcccaaccagcctcaagtttgggagctgatacaatgtc |
| | | | tgcgggaggtggcggcccattgggcgacaataaccaaggtgccgatggagtggg |
| | | | caatgcctcgggagattcgcattgcgattccacgtggatgggggacagagtcgtca |
| | | | ccaagtccacccgaacctgggtgctgcccagctacaacaaccaccagtaccgaga |
| | | | gatcaaaagcggctccgtcgacggaagcaacgccaacgcctactttggatacagca |
| | | | ccccctgggggtactttgactttaaccgcttccacagccactggagcccccgagact |
| | | | ggcaaagactcatcaacaactactggggcttcagaccccggtccctcagagtcaaa |
| | | | atcttcaacattcaagtcaaagaggtcacggtgcaggactccaccaccaccatcgcc |
| | | | aacaacctcacctccaccgtccaagtgtttacggacgacgactaccagctgccctac |
| | | | gtcgtcggcaacgggaccgagggatgcctgccggccttccctccgcaggtctttac |
| | | | gctgccgcagtacgcttacgcgacgctgaaccgcgacaacacagaaaatcccacc |
| | | | gagaggagcagcttcttctgcctagagtactttcccagcaagatgctgagaacgggc |
| | | | aacaactttgagtttacctacaactttgaggaggtgcccttccactccagcttcgctccc |
| | | | agtcagaacctgttcaagctggccaacccgctggtggaccagtacttgtaccgcttc |
| | | | gtgagcacaaataacactggcggagtccagttcaacaagaacctggccgggagat |
| | | | acgccaacacctacaaaaactggttcccggggcccatgggccgaacccagggctg |
| | | | gaacctgggctccggggtcaaccgcgccagtgtcagcgccttcgccacgaccaat |
| | | | aggatggagctcgagggcgcgagttaccaggtgcccccgcagccgaacggcatg |
| | | | accaacaacctccagggcagcaacacctatgccctggagaacactatgatcttcaac |
| | | | agccagccggcgaacccgggcaccaccgccacgtacctcgagggcaacatgctc |
| | | | atcaccagcgagagcgagacgcagccggtgaaccgcgtggcgtacaacgtcggc |
| | | | gggcagatggccaccaacaaccagagctccaccactgcccccgcgaccggcacg |
| | | | tacaacctccaggaaatcgtgcccggcagcgtgtcgatcgagagggacgtgtacct |
| | | | ccaaggacccatctgggccaagatcccagagacgggggcgcactttcacccctctc |
| | | | cggccatgggcggattcggactcaaacacccaccgcccatgatgctcatcaagaac |
| | | | acgcctgtgcccggaaatatcaccagcttctcggacgtgcccgtcagcagcttcatc |
| | | | acccagtacagcaccgggcaggtcaccgtggagatggagtgggagctcaagaag |
| | | | gaaaactccaagaggtggaacccagagatccagtacacaaacaactacaacgacc |
| | | | cccagtttgtggactttgccccggacagcaccggggaatacagaaccaccagacct |
| | | | atcggaacccgataccttacccgacccctttaaTTGCTTGTTAATCAA |
| | | | TAAACCGTTTAATTCGTTTCAGTTGAACTTTGGTC |
| | | | TCTGCCCTAGGGGAGGTCCTGTATTAGAGGTCAC |
| | | | GTGAGTGTTTTGCGACATTTTGCGACACCATGT |
| | | | GGTCACGCTGGGTATTTAAGCCCGAGTGAGCAC |
| | | | GCAGGGTCTCCATTTTGAAGCGGGAGGTTTGAA |
| | | | CATACAGACGAAACTAGTTGGAGATTATCGAGCAT |
| | | | TCTATCACGTCGACGACCACTAGTGAGTTACTAGA |
| | | | GCCGAGGGGTAACGTTGATGCTCCTAAAAATCTCT |
| | | | CGGTCGATGCAAGCGATTACACTCCTGTCACATCA |
| | | | TAATCGTTTGCTATTCAGGGCTTGATCAACACTGGA |
| | | | TTGCTTTTCACTTAAAGTATTATGCACGACAGGGTA |
| | | | CGTGTACCATATAAACCTGTTATAACTTACCTCAGA |
| | | | CTAGTTGGAAATGTGGCTAGATCTTAGCTTACGTTA |
| | | | CTAAAGGGTCCACGTTgaggtcctgtattagaggtcacgtgagtgt |
| | | | tttgcgacattttgcgacaccatgtggtcacgctgggtatttaagcccgagtgag |
| | | | cacgcagggtctccattttgaagcgggaggtttgaaATCCCAATGGCG |
| | | | CGCCGAGCTTGGCTCGAGCATGGTCATAGCTGTTTC |
| | | | CTGTGTGAAATTGTTATCCGCTCACAATTCCACACA |
| | | | ACATACGAGCCGGAAGCATAAAGTGTAAAGCCTGG |
| | | | GGTGCCTAATGAGTGAGCTAACTCACATTAATTGC |
| | | | GTTGCGCTCACTGCCCGCTTTCCAGTCGGGAAACCT |
| | | | GTCGTGCCAGCTGCATTAATGAATCGGCCAACGCG |
| | | | CGGGGAGAGGCGGTTTGCGTATTGGGCGCTCTTCC |
| | | | GCTTCCTCGCTCACTGACTCGCTGCGCTCGGTCGTT |
| | | | CGGCTGCGGCGAGCGGTATCAGCTCACTCAAAGGC |
| | | | GGTAATACGGTTATCCACAGAATCAGGGGATAACG |
| | | | CAGGAAAGAACATGTGAGCAAAAGGCCAGCAAAA |
| | | | GGCCAGGAACCGTAAAAAGGCCGCGTTGCTGGCGT |
| | | | TTTTCCATAGGCTCCGCCCCCCTGACGAGCATCACA |
| | | | AAAATCGACGCTCAAGTCAGAGGTGGCGAAACCCG |
| | | | ACAGGACTATAAAGATACCAGGCGTTTCCCCCTGG |
| | | | AAGCTCCCTCGTGCGCTCTCCTGTTCCGACCCTGCC |
| | | | GCTTACCGGATACCTGTCCGCCTTTCTCCCTTCGGG |
| | | | AAGCGTGGCGCTTTCTCATAGCTCACGCTGTAGGT |
| | | | ATCTCAGTTCGGTGTAGGTCGTTCGCTCCAAGCTGG |
| | | | GCTGTGTGCACGAACCCCCCGTTCAGCCCGACCGC |
| | | | TGCGCCTTATCCGGTAACTATCGTCTTGAGTCCAAC |
| | | | CCGGTAAGACACGACTTATCGCCACTGGCAGCAGC |
| | | | CACTGGTAACAGGATTAGCAGAGCGAGGTATGTAG |
| | | | GCGGTGCTACAGAGTTCTTGAAGTGGTGGCCTAAC |
| | | | TACGGCTACACTAGAAGAACAGTATTTGGTATCTG |
| | | | CGCTCTGCTGAAGCCAGTTACCTTCGGAAAAAGAG |
| | | | TTGGTAGCTCTTGATCCGGCAAACAAACCACCGCT |
| | | | GGTAGCGGTGGTTTTTTTGTTTGCAAGCAGCAGATT |
| | | | ACGCGCAGAAAAAAAGGATCTCAAGAAGATCCTTT |
| | | | GATCTTTTCTACGGGGTCTGACGCTCAGTGGAACG |
| | | | AAAACTCACGTTAAGGGATTTTGGTCATGAGATTA |
| | | | TCAAAAAGGATCTTCACCTAGATCCTTTTAAATTAA |
| | | | AAATGAAGTTTTAAATCAATCTAAAGTATATATGA |
| | | | GTAAACTTGGTCTGACAGTTAGAAAAACTCATCGA |
| | | | GCATCAAATGAAACTGCAATTTATTCATATCAGGA |
| | | | TTATCAATACCATATTTTTGAAAAAGCCGTTTCTGT |
| | | | AATGAAGGAGAAAACTCACCGAGGCAGTTCCATAG |
| | | | GATGGCAAGATCCTGGTATCGGTCTGCGATTCCGA |
| | | | CTCGTCCAACATCAATACAACCTATTAATTTCCCCT |
| | | | CGTCAAAAATAAGGTTATCAAGTGAGAAATCACCA |
| | | | TGAGTGACGACTGAATCCGGTGAGAATGGCAAAAG |
| | | | TTTATGCATTTCTTTCCAGACTTGTTCAACAGGCCA |
| | | | GCCATTACGCTCGTCATCAAAATCACTCGCATCAA |
| | | | CCAAACCGTTATTCATTCGTGATTGCGCCTGAGCGA |
| | | | GACGAAATACGCGATCGCTGTTAAAAGGACAATTA |
| | | | CAAACAGGAATCGAATGCAACCGGCGCAGGAACA |
| | | | CTGCCAGCGCATCAACAATATTTTCACCTGAATCA |
| | | | GGATATTCTTCTAATACCTGGAATGCTGTTTTCCCA |
| | | | GGGATCGCAGTGGTGAGTAACCATGCATCATCAGG |
| | | | AGTACGGATAAAATGCTTGATGGTCGGAAGAGGCA |
| | | | TAAATTCCGTCAGCCAGTTTAGTCTGACCATCTCAT |
| | | | CTGTAACATCATTGGCAACGCTACCTTTGCCATGTT |
| | | | TCAGAAACAACTCTGGCGCATCGGGCTTCCCATAC |
| | | | AATCGATAGATTGTCGCACCTGATTGCCCGACATT |
| | | | ATCGCGAGCCCATTTATACCCATATAAATCAGCAT |
| | | | CCATGTTGGAATTTAATCGCGGCCTAGAGCAAGAC |
| | | | GTTTCCCGTTGAATATGGCTCATACTCTTCCTTTTTC |
| | | | AATATTATTGAAGCATTTATCAGGGTTATTGTCTCA |
| | | | TGAGCGGATACATATTTGAATGTATTTAGAAAAAT |
| | | | AAACAAATAGGGGTTCCGCGCACATTTCCCCGAAA |
| | | | AGTGCCACCTGACGTCTAAGAAACCATTATTATCA |
| | | | TGACATTAACCTATAAAAATAGGCGTATCACGAGG |
| | | | CCCTTTCGTC |
| 62 | pYZZ441 | Rep2Cap9- | ATGCCGGGGTTTTACGAGATTGTGATTAAGGTCCCCAG |
| | | p5.p5.p5 | CGACCTTGACGAGCATCTGCCCGGCATTTCTGACAGC |
| | | | TTTGTGAACTGGGTGGCCGAGAAGGAATGGGAGTTGC |
| | | | CGCCAGATTCTGACATGGATCTGAATCTGATTGAGCAG |
| | | | GCACCCCTGACCGTGGCCGAGAAGCTGCAGCGCGAC |
| | | | TTTCTGACGGAATGGCGCCGTGTGAGTAAGGCCCCGG |
| | | | AGGCTCTTTTCTTTGTGCAATTTGAGAAGGGAGAGAGC |
| | | | TACTTCCACATGCACGTGCTCGTGGAAACCACCGGGG |
| | | | TGAAATCCATGGTTTTGGGACGTTTCCTGAGTCAGATT |
| | | | CGCGAAAAACTGATTCAGAGAATTTACCGCGGGATCG |
| | | | AGCCGACTTTGCCAAACTGGTTCGCGGTCACAAAGAC |
| | | | CAGAAATGGCGCCGGAGGCGGGAACAAGGTGGTGGA |
| | | | TGAGTGCTACATCCCCAATTACTTGCTCCCCAAAACCC |
| | | | AGCCTGAGCTCCAGTGGGCGTGGACTAATATGGAACA |
| | | | GTATTTAAGCGCCTGTTTGAATCTCACGGAGCGTAAAC |
| | | | GGTTGGTGGCGCAGCATCTGACGCACGTGTCGCAGAC |
| | | | GCAGGAGCAGAACAAAGAGAATCAGAATCCCAATTCT |
| | | | GATGCGCCGGTGATCAGATCAAAAACTTCAGCCAGGT |
| | | | ACATGGAGCTGGTCGGGTGGCTCGTGGACAAGGGGA |
| | | | TTACCTCGGAGAAGCAGTGGATCCAGGAGGACCAGGC |
| | | | CTCATACATCTCCTTCAATGCGGCCTCCAACTCGCGGT |
| | | | CCCAAATCAAGGCTGCCTTGGACAATGCGGGAAAGAT |
| | | | TATGAGCCTGACTAAAACCGCCCCCGACTACCTGGTG |
| | | | GGCCAGCAGCCCGTGGAGGACATTTCCAGCAATCGGA |
| | | | TTTATAAAATTTTGGAACTAAACGGGTACGATCCCCAAT |
| | | | ATGCGGCTTCCGTCTTTCTGGGATGGGCCACGAAAAA |
| | | | GTTCGGCAAGAGGAACACCATCTGGCTGTTTGGGCCT |
| | | | GCAACTACCGGGAAGACCAACATCGCGGAGGCCATAG |
| | | | CCCACACTGTGCCCTTCTACGGGTGCGTAAACTGGAC |
| | | | CAATGAGAACTTTCCCTTCAACGACTGTGTCGACAAGA |
| | | | TGGTGATCTGGTGGGAGGAGGGGAAGATGACCGCCA |
| | | | AGGTCGTGGAGTCGGCCAAAGCCATTCTCGGAGGAAG |
| | | | CAAGGTGCGCGTGGACCAGAAATGCAAGTCCTCGGCC |
| | | | CAGATAGACCCGACTCCCGTGATCGTCACCTCCAACA |
| | | | CCAACATGTGCGCCGTGATTGACGGGAACTCAACGAC |
| | | | CTTCGAACACCAGCAGCCGTTGCAAGACCGGATGTTC |
| | | | AAATTTGAACTCACCCGCCGTCTGGATCATGACTTTGG |
| | | | GAAGGTCACCAAGCAGGAAGTCAAAGACTTTTTCCGGT |
| | | | GGGCAAAGGATCACGTGGTTGAGGTGGAGCATGAATT |
| | | | CTACGTCAAAAAGGGTGGAGCCAAGAAAAGACCCGCC |
| | | | CCCAGTGACGCAGATATAAGTGAGCCCAAACGGGTGC |
| | | | GCGAGTCAGTIGCGCAGCCATCGACGTCAGACGCGG |
| | | | AAGCTTCGATCAACTACGCAGACAGGTACCAAAACAAA |
| | | | TGTTCTCGTCACGTGGGCATGAATCTGATGCTGTTTCC |
| | | | CTGCAGACAATGCGAGAGAATGAATCAGAATTCAAATA |
| | | | TCTGCTTCACTCACGGACAGAAAGACTGTTTAGAGTGC |
| | | | TTTCCCGTGTCAGAATCTCAACCCGTTTCTGTCGTCAA |
| | | | AAAGGCGTATCAGAAACTGTGCTACATTCATCATATCAT |
| | | | GGGAAAGGTGCCAGACGCTTGCACTGCCTGCGATCTG |
| | | | GTCAATGTGGATTTGGATGACTGCATCTTTGAACAATA |
| | | | AATGATTTAAATCAGGTatgtcttttgttgatcaccctccagattggttg |
| | | | gaagaagttggtgaaggtcttcgcgagtttttgggccttgaagcgggcccaccgaaa |
| | | | ccaaaacccaatcagcagcatcaagatcaagcccgtggtcttgtgctgcctggttata |
| | | | actatctcggacccggaaacggtctcgatcgaggagagcctgtcaacagggcaga |
| | | | cgaggtcgcgcgagagcacgacatctcgtacaacgagcagcttgaggcgggaga |
| | | | caacccctacctcaagtacaaccacgcggacgccgagtttcaggagaagctcgcc |
| | | | gacgacacatccttcgggggaaacctcggaaaggcagtctttcaggccaagaaaa |
| | | | gggttctcgaaccttttggcctgcttgaagagggtgctaagacggcccctaccggaa |
| | | | agcggatagacgaccactttccaaaaagaaagaaggctcggaccgaagaggactc |
| | | | caagccttccacctcgtcagacgccgaagctggacccagcggatcccagcagctg |
| | | | caaatcccagcccaaccagcctcaagtttgggagctgatacaatgtctgcgggaggt |
| | | | ggcggcccattgggcgacaataaccaaggtgccgatggagtgggcaatgcctcgg |
| | | | gagattggcattgcgattccacgtggatgggggacagagtcgtcaccaagtccacc |
| | | | cgaacctgggtgctgcccagctacaacaaccaccagtaccgagagatcaaaagcg |
| | | | gctccgtcgacggaagcaacgccaacgcctactttggatacagcaccccctgggg |
| | | | gtactttgactttaaccgcttccacagccactggagcccccgagactggcaaagact |
| | | | catcaacaactactggggcttcagaccccggtccctcagagtcaaaatcttcaacatt |
| | | | caagtcaaagaggtcacggtgcaggactccaccaccaccatcgccaacaacctca |
| | | | cctccaccgtccaagtgtttacggacgacgactaccagctgccctacgtcgtcggca |
| | | | acgggaccgagggatgcctgccggccttccctccgcaggtctttacgctgccgcag |
| | | | tacggttacgcgacgctgaaccgcgacaacacagaaaatcccaccgagaggagca |
| | | | gcttcttctgcctagagtactttcccagcaagatgctgagaacgggcaacaactttga |
| | | | gtttacctacaactttgaggaggtgcccttccactccagcttcgctcccagtcagaacc |
| | | | tgttcaagctggccaacccgctggtggaccagtacttgtaccgcttcgtgagcacaa |
| | | | ataacactggcggagtccagttcaacaagaacctggccgggagatacgccaacac |
| | | | ctacaaaaactggttcccggggcccatgggccgaacccagggctggaacctgggc |
| | | | tccggggtcaaccgcgccagtgtcagcgccttcgccacgaccaataggatggagct |
| | | | cgagggcgcgagttaccaggtgcccccgcagccgaacggcatgaccaacaacct |
| | | | ccagggcagcaacacctatgccctggagaacactatgatcttcaacagccagccgg |
| | | | cgaacccgggcaccaccgccacgtacctcgagggcaacatgctcatcaccagcga |
| | | | gagcgagacgcagccggtgaaccgcgtggcgtacaacgtcggcgggcagatgg |
| | | | ccaccaacaaccagagctccaccactgcccccgcgaccggcacgtacaacctcca |
| | | | ggaaatcgtgcccggcagcgtgtcgatggagagggacgtgtacctccaaggaccc |
| | | | atctgggccaagatcccagagacgggggcgcactttcacccctctccggccatggg |
| | | | cggattcggactcaaacacccaccgcccatgatgctcatcaagaacacgcctgtgc |
| | | | ccggaaatatcaccagcttctcggacgtgcccgtcagcagcttcatcacccagtaca |
| | | | gcaccgggcaggtcaccgtggagatggagtgggagctcaagaaggaaaactcca |
| | | | agaggtggaacccagagatccagtacacaaacaactacaacgacccccagtttgtg |
| | | | gactttgccccggacagcaccggggaatacagaaccaccagacctatcggaaccc |
| | | | gataccttacccgacccctttaaTTGCTTGTTAATCAATAAACC |
| | | | GTTTAATTCGTTTCAGTTGAACTTTGGTCTCTGCC |
| | | | CTAGGGGAGGTCCTGTATTAGAGGTCACGTGAG |
| | | | TGTTTTGCGACATTTTGCGACACCATGTGGTCAC |
| | | | GCTGGGTATTTAAGCCCGAGTGAGCACGCAGGG |
| | | | TCTCCATTTTGAAGCGGGAGGTTTGAAcgcgcagccg |
| | | | ccaagccgtttcagttgaactttggtctctgcgtatttctttcttatctagtttccatggag |
| | | | gtcctgtattagaggtcacgtgagtgttttgcgacattttgcgacaccatgtggtc |
| | | | acgctgggtatttaagcccgagtgagcacgcagggtctccattttgaagcggga |
| | | | ggtttgaacgcgcagccgccaagccgtttcagttgaactttggtctctgcgtatttcttt |
| | | | cttatctagtttccatggaggtcctgtattagaggtcacgtgagtgttttgcgacatt |
| | | | ttgcgacaccatgtggtcacgctgggtatttaagcccgagtgagcacgcagggt |
| | | | ctccattttgaagcgggaggtttgaaATCCCAATGGCGCGCCGA |
| | | | GCTTGGCTCGAGCATGGTCATAGCTGTTTCCTGTGT |
| | | | GAAATTGTTATCCGCTCACAATTCCACACAACATA |
| | | | CGAGCCGGAAGCATAAAGTGTAAAGCCTGGGGTGC |
| | | | CTAATGAGTGAGCTAACTCACATTAATTGCGTTGC |
| | | | GCTCACTGCCCGCTTTCCAGTCGGGAAACCTGTCGT |
| | | | GCCAGCTGCATTAATGAATCGGCCAACGCGCGGGG |
| | | | AGAGGCGGTTTGCGTATTGGGCGCTCTTCCGCTTCC |
| | | | TCGCTCACTGACTCGCTGCGCTCGGTCGTTCGGCTG |
| | | | CGGCGAGCGGTATCAGCTCACTCAAAGGCGGTAAT |
| | | | ACGGTTATCCACAGAATCAGGGGATAACGCAGGAA |
| | | | AGAACATGTGAGCAAAAGGCCAGCAAAAGGCCAG |
| | | | GAACCGTAAAAAGGCCGCGTTGCTGGCGTTTTTCC |
| | | | ATAGGCTCCGCCCCCCTGACGAGCATCACAAAAAT |
| | | | CGACGCTCAAGTCAGAGGTGGCGAAACCCGACAG |
| | | | GACTATAAAGATACCAGGCGTTTCCCCCTGGAAGC |
| | | | TCCCTCGTGCGCTCTCCTGTTCCGACCCTGCCGCTT |
| | | | ACCGGATACCTGTCCGCCTTTCTCCCTTCGGGAAGC |
| | | | GTGGCGCTTTCTCATAGCTCACGCTGTAGGTATCTC |
| | | | AGTTCGGTGTAGGTCGTTCGCTCCAAGCTGGGCTGT |
| | | | GTGCACGAACCCCCCGTTCAGCCCGACCGCTGCGC |
| | | | CTTATCCGGTAACTATCGTCTTGAGTCCAACCCGGT |
| | | | AAGACACGACTTATCGCCACTGGCAGCAGCCACTG |
| | | | GTAACAGGATTAGCAGAGCGAGGTATGTAGGCGGT |
| | | | GCTACAGAGTTCTTGAAGTGGTGGCCTAACTACGG |
| | | | CTACACTAGAAGAACAGTATTTGGTATCTGCGCTCT |
| | | | GCTGAAGCCAGTTACCTTCGGAAAAAGAGTTGGTA |
| | | | GCTCTTGATCCGGCAAACAAACCACCGCTGGTAGC |
| | | | GGTGGTTTTTTTGTTTGCAAGCAGCAGATTACGCGC |
| | | | AGAAAAAAAGGATCTCAAGAAGATCCTTTGATCTT |
| | | | TTCTACGGGGTCTGACGCTCAGTGGAACGAAAACT |
| | | | CACGTTAAGGGATTTTGGTCATGAGATTATCAAAA |
| | | | AGGATCTTCACCTAGATCCTTTTAAATTAAAAATGA |
| | | | AGTTTTAAATCAATCTAAAGTATATATGAGTAAAC |
| | | | TTGGTCTGACAGTTAGAAAAACTCATCGAGCATCA |
| | | | AATGAAACTGCAATTTATTCATATCAGGATTATCA |
| | | | ATACCATATTTTTGAAAAAGCCGTTTCTGTAATGAA |
| | | | GGAGAAAACTCACCGAGGCAGTTCCATAGGATGGC |
| | | | AAGATCCTGGTATCGGTCTGCGATTCCGACTCGTCC |
| | | | AACATCAATACAACCTATTAATTTCCCCTCGTCAAA |
| | | | AATAAGGTTATCAAGTGAGAAATCACCATGAGTGA |
| | | | CGACTGAATCCGGTGAGAATGGCAAAAGTTTATGC |
| | | | ATTTCTTTCCAGACTTGTTCAACAGGCCAGCCATTA |
| | | | CGCTCGTCATCAAAATCACTCGCATCAACCAAACC |
| | | | GTTATTCATTCGTGATTGCGCCTGAGCGAGACGAA |
| | | | ATACGCGATCGCTGTTAAAAGGACAATTACAAACA |
| | | | GGAATCGAATGCAACCGGCGCAGGAACACTGCCA |
| | | | GCGCATCAACAATATTTTCACCTGAATCAGGATATT |
| | | | CTTCTAATACCTGGAATGCTGTTTTCCCAGGGATCG |
| | | | CAGTGGTGAGTAACCATGCATCATCAGGAGTACGG |
| | | | ATAAAATGCTTGATGGTCGGAAGAGGCATAAATTC |
| | | | CGTCAGCCAGTTTAGTCTGACCATCTCATCTGTAAC |
| | | | ATCATTGGCAACGCTACCTTTGCCATGTTTCAGAAA |
| | | | CAACTCTGGCGCATCGGGCTTCCCATACAATCGAT |
| | | | AGATTGTCGCACCTGATTGCCCGACATTATCGCGA |
| | | | GCCCATTTATACCCATATAAATCAGCATCCATGTTG |
| | | | GAATTTAATCGCGGCCTAGAGCAAGACGTTTCCCG |
| | | | TTGAATATGGCTCATACTCTTCCTTTTTCAATATTAT |
| | | | TGAAGCATTTATCAGGGTTATTGTCTCATGAGCGG |
| | | | ATACATATTTGAATGTATTTAGAAAAATAAACAAA |
| | | | TAGGGGTTCCGCGCACATTTCCCCGAAAAGTGCCA |
| | | | CCTGACGTCTAAGAAACCATTATTATCATGACATTA |
| | | | ACCTATAAAAATAGGCGTATCACGAGGCCCTTTCG |
| | | | TCTCGCGCGTTTCGGTGATGACGGTGAAAACCTCT |
| | | | GACACATGCAGCTCCCGGAGACGGTCACAGCTTGT |
| | | | CTGTAAGCGGATGCCGGGAGCAGACAAGCCCGTCA |
| | | | GGGCGCGTCAGCGGGTGTTGGCGGGTGTCGGGGCT |
| | | | GGCTTAACTATGCGGCATCAGAGCAGATTGTACTG |
| | | | AGAGTGCACCATATGCGGTGTGAAATACCGCACAG |
| | | | ATGCGTAAGGAGAAAATACCGCATCAGGCGCCATT |
| | | | CGCCATTCAGGCTGCGCAACTGTTGGGAAGGGCGA |
| | | | TCGGTGCGGGCCTCTTCGCTATTACGCCAGCTGGCG |
| | | | AAAGGGGGATGTGCTGCAAGGCGATTAAGTTGGGT |
| | | | AACGCCAGGGTTTTCCCAGTCACGACGTTGTAAAA |
| | | | CGACGGCCAGTGAATTGACGCGTATTGGGATCGCG |
| | | | CAGCCGCC |
| 63 | pYZZ314 | R2C2-p5 | TCGCGCGTTTCGGTGATGACGGTGAAAACCTCTGACACAT |
| | | | GCAGCTCCCGGAGACGGTCACAGCTTGTCTGTAAGCGGAT |
| | | | GCCGGGAGCAGACAAGCCCGTCAGGGCGCGTCAGCGGGTG |
| | | | TTGGCGGGTGTCGGGGCTGGCTTAACTATGCGGCATCAGA |
| | | | GCAGATTGTACTGAGAGTGCACCATATGCGGTGTGAAATA |
| | | | CCGCACAGATGCGTAAGGAGAAAATACCGCATCAGGCGCC |
| | | | ATTCGCCATTCAGGCTGCGCAACTGTTGGGAAGGGCGATC |
| | | | GGTGCGGGCCTCTTCGCTATTACGCCAGCTGGCGAAAGGG |
| | | | GGATGTGCTGCAAGGCGATTAAGTTGGGTAACGCCAGGGT |
| | | | TTTCCCAGTCACGACGTTGTAAAACGACGGCCAGTGAATT |
| | | | GACGCGTATTGGGATCGCGCAGCCGCCATGCCGGGGTTTT |
| | | | ACGAGATTGTGATTAAGGTCCCCAGCGACCTTGACGAGCA |
| | | | TCTGCCCGGCATTTCTGACAGCTTTGTGAACTGGGTGGCCG |
| | | | AGAAGGAATGGGAGTTGCCGCCAGATTCTGACATGGATCT |
| | | | GAATCTGATTGAGCAGGCACCCCTGACCGTGGCCGAGAAG |
| | | | CTGCAGCGCGACTTTCTGACGGAATGGCGCCGTGTGAGTA |
| | | | AGGCCCCGGAGGCTCTTTTCTTTGTGCAATTTGAGAAGGG |
| | | | AGAGAGCTACTTCCACATGCACGTGCTCGTGGAAACCACC |
| | | | GGGGTGAAATCCATGGTTTTGGGACGTTTCCTGAGTCAGA |
| | | | TTCGCGAAAAACTGATTCAGAGAATTTACCGCGGGATCGA |
| | | | GCCGACTTTGCCAAACTGGTTCGCGGTCACAAAGACCAGA |
| | | | AATGGCGCCGGAGGCGGGAACAAGGTGGTGGATGAGTGCT |
| | | | ACATCCCCAATTACTTGCTCCCCAAAACCCAGCCTGAGCTC |
| | | | CAGTGGGCGTGGACTAATATGGAACAGTATTTAAGCGCCT |
| | | | GTTTGAATCTCACGGAGCGTAAACGGTTGGTGGCGCAGCA |
| | | | TCTGACGCACGTGTCGCAGACGCAGGAGCAGAACAAAGAG |
| | | | AATCAGAATCCCAATTCTGATGCGCCGGTGATCAGATCAA |
| | | | AAACTTCAGCCAGGTACATGGAGCTGGTCGGGTGGCTCGT |
| | | | GGACAAGGGGATTACCTCGGAGAAGCAGTGGATCCAGGAG |
| | | | GACCAGGCCTCATACATCTCCTTCAATGCGGCCTCCAACTC |
| | | | GCGGTCCCAAATCAAGGCTGCCTTGGACAATGCGGGAAAG |
| | | | ATTATGAGCCTGACTAAAACCGCCCCCGACTACCTGGTGG |
| | | | GCCAGCAGCCCGTGGAGGACATTTCCAGCAATCGGATTTA |
| | | | TAAAATTTTGGAACTAAACGGGTACGATCCCCAATATGCG |
| | | | GCTTCCGTCTTTCTGGGATGGGCCACGAAAAAGTTCGGCA |
| | | | AGAGGAACACCATCTGGCTGTTTGGGCCTGCAACTACCGG |
| | | | GAAGACCAACATCGCGGAGGCCATAGCCCACACTGTGCCCT |
| | | | TCTACGGGTGCGTAAACTGGACCAATGAGAACTTTCCCTT |
| | | | CAACGACTGTGTCGACAAGATGGTGATCTGGTGGGAGGAG |
| | | | GGGAAGATGACCGCCAAGGTCGTGGAGTCGGCCAAAGCCA |
| | | | TTCTCGGAGGAAGCAAGGTGCGCGTGGACCAGAAATGCAA |
| | | | GTCCTCGGCCCAGATAGACCCGACTCCCGTGATCGTCACCT |
| | | | CCAACACCAACATGTGCGCCGTGATTGACGGGAACTCAAC |
| | | | GACCTTCGAACACCAGCAGCCGTTGCAAGACCGGATGTTC |
| | | | AAATTTGAACTCACCCGCCGTCTGGATCATGACTTTGGGA |
| | | | AGGTCACCAAGCAGGAAGTCAAAGACTTTTTCCGGTGGGC |
| | | | AAAGGATCACGTGGTTGAGGTGGAGCATGAATTCTACGTC |
| | | | AAAAAGGGTGGAGCCAAGAAAAGACCCGCCCCCAGTGACG |
| | | | CAGATATAAGTGAGCCCAAACGGGTGCGCGAGTCAGTTGC |
| | | | GCAGCCATCGACGTCAGACGCGGAAGCTTCGATCAACTAC |
| | | | GCAGACAGGTACCAAAACAAATGTTCTCGTCACGTGGGCA |
| | | | TGAATCTGATGCTGTTTCCCTGCAGACAATGCGAGAGAAT |
| | | | GAATCAGAATTCAAATATCTGCTTCACTCACGGACAGAAA |
| | | | GACTGTTTAGAGTGCTTTCCCGTGTCAGAATCTCAACCCGT |
| | | | TTCTGTCGTCAAAAAGGCGTATCAGAAACTGTGCTACATT |
| | | | CATCATATCATGGGAAAGGTGCCAGACGCTTGCACTGCCT |
| | | | GCGATCTGGTCAATGTGGATTTGGATGACTGCATCTTTGA |
| | | | ACAATAAATGATTTAAATCAGGTatggctgccgatggttatcttcca |
| | | | gattggctcgaggacactctctctgaaggaataagacagtggtggaagctcaaa |
| | | | cctggcccaccaccaccaaagcccgcagagcggcataaggacgacagcaggg |
| | | | gtcttgtgcttcctgggtacaagtacctcggacccttcaacggactcgacaaggg |
| | | | agagccggtcaacgaggcagacgccgcggccctcgagcacgacaaagcctac |
| | | | gaccggcagctcgacagcggagacaacccgtacctcaagtacaaccacgccg |
| | | | acgcggagtttcaggagcgccttaaagaagatacgtcttttgggggcaacctcg |
| | | | gacgagcagtcttccaggcgaaaaagagggttcttgaacctctgggcctggttg |
| | | | aggaacctgttaagacggctccgggaaaaaagaggccggtagagcactctcct |
| | | | gtggagccagactcctcctcgggaaccggaaaggcgggccagcagcctgcaa |
| | | | gaaaaagattgaattttggtcagactggagacgcagactcagtacctgaccccc |
| | | | agcctctcggacagccaccagcagccccctctggtctgggaactaatacgatgg |
| | | | ctacaggcagtggcgcaccaatggcagacaataacgagggcgccgacggagt |
| | | | gggtaattcctcgggaaattggcattgcgattccacatggatgggcgacagagt |
| | | | catcaccaccagcacccgaacctgggccctgcccacctacaacaaccacctcta |
| | | | caaacaaatttccagccaatcaggagcctcgaacgacaatcactactttggcta |
| | | | cagcaccccttgggggtattttgacttcaacagattccactgccacttttcaccac |
| | | | gtgactggcaaagactcatcaacaacaactggggattccgacccaagagactc |
| | | | aacttcaagctctttaacattcaagtcaaagaggtcacgcagaatgacggtacg |
| | | | acgacgattgccaataaccttaccagcacggttcaggtgtttactgactcggagt |
| | | | accagctcccgtacgtcctcggctcggcgcatcaaggatgcctcccgccgttccc |
| | | | agcagacgtcttcatggtgccacagtatggatacctcaccctgaacaacgggag |
| | | | tcaggcagtaggacgctcttcattttactgcctggagtactttccttctcagatgct |
| | | | gcgtaccggaaacaactttaccttcagctacacttttgaggacgttcctttccaca |
| | | | gcagctacgctcacagccagagtctggaccgtctcatgaatcctctcatcgacca |
| | | | gtacctgtattacttgagcagaacaaacactccaagtggaaccaccacgcagtc |
| | | | aaggcttcagttttctcaggccggagcgagtgacattcgggaccagtctaggaa |
| | | | ctggcttcctggaccctgttaccgccagcagcgagtatcaaagacatctgcggat |
| | | | aacaacaacagtgaatactcgtggactggagctaccaagtaccacctcaatggc |
| | | | agagactctctggtgaatccgggcccggccatggcaagccacaaggacgatga |
| | | | agaaaagttttttcctcagagcggggttctcatctttgggaagcaaggctcagag |
| | | | aaaacaaatgtggacattgaaaaggtcatgattacagacgaagaggaaatcag |
| | | | gacaaccaatcccgtggctacggagcagtatggttctgtatctaccaacctccag |
| | | | agaggcaacagacaagcagctaccgcagatgtcaacacacaaggcgttcttcc |
| | | | aggcatggtctggcaggacagagatgtgtaccttcaggggcccatctgggcaa |
| | | | agattccacacacggacggacattttcacccctctcccctcatgggtggattcgg |
| | | | acttaaacaccctcctccacagattctcatcaagaacaccccggtacctgcgaat |
| | | | ccttcgaccaccttcagtgcggcaaagtttgcttccttcatcacacagtactccac |
| | | | gggacaggtcagcgtggagatcgagtgggagctgcagaaggaaaacagcaa |
| | | | acgctggaatcccgaaattcagtacacttccaactacaacaagtctgttaatgtg |
| | | | gactttactgtggacactaatggcgtgtattcagagcctcgccccattggcacca |
| | | | gatacctgactcgtaatctgtaaTTGCTTGTTAATCAATAAACCGTT |
| | | | TAATTCGTTTCAGTTGAACTTTGGTCTCTGCGGAGGTCCT |
| | | | GTATTAGAGGTCACGTGAGTGTTTTGCGACATTTTGCGAC |
| | | | ACCATGTGGTCACGCTGGGTATTTAAGCCCGAGTGAGCAC |
| | | | GCAGGGTCTCCATTTTGAAGCGGGAGGTTTGAAATCCCAA |
| | | | TGGCGCGCCGAGCTTGGCTCGAGCATGGTCATAGCTGTTTC |
| | | | CTGTGTGAAATTGTTATCCGCTCACAATTCCACACAACAT |
| | | | ACGAGCCGGAAGCATAAAGTGTAAAGCCTGGGGTGCCTAA |
| | | | TGAGTGAGCTAACTCACATTAATTGCGTTGCGCTCACTGC |
| | | | CCGCTTTCCAGTCGGGAAACCTGTCGTGCCAGCTGCATTAA |
| | | | TGAATCGGCCAACGCGCGGGGAGAGGCGGTTTGCGTATTG |
| | | | GGCGCTCTTCCGCTTCCTCGCTCACTGACTCGCTGCGCTCG |
| | | | GTCGTTCGGCTGCGGCGAGCGGTATCAGCTCACTCAAAGG |
| | | | CGGTAATACGGTTATCCACAGAATCAGGGGATAACGCAGG |
| | | | AAAGAACATGTGAGCAAAAGGCCAGCAAAAGGCCAGGAAC |
| | | | CGTAAAAAGGCCGCGTTGCTGGCGTTTTTCCATAGGCTCC |
| | | | GCCCCCCTGACGAGCATCACAAAAATCGACGCTCAAGTCA |
| | | | GAGGTGGCGAAACCCGACAGGACTATAAAGATACCAGGCG |
| | | | TTTCCCCCTGGAAGCTCCCTCGTGCGCTCTCCTGTTCCGAC |
| | | | CCTGCCGCTTACCGGATACCTGTCCGCCTTTCTCCCTTCGG |
| | | | GAAGCGTGGCGCTTTCTCATAGCTCACGCTGTAGGTATCTC |
| | | | AGTTCGGTGTAGGTCGTTCGCTCCAAGCTGGGCTGTGTGC |
| | | | ACGAACCCCCCGTTCAGCCCGACCGCTGCGCCTTATCCGGT |
| | | | AACTATCGTCTTGAGTCCAACCCGGTAAGACACGACTTAT |
| | | | CGCCACTGGCAGCAGCCACTGGTAACAGGATTAGCAGAGC |
| | | | GAGGTATGTAGGCGGTGCTACAGAGTTCTTGAAGTGGTGG |
| | | | CCTAACTACGGCTACACTAGAAGAACAGTATTTGGTATCT |
| | | | GCGCTCTGCTGAAGCCAGTTACCTTCGGAAAAAGAGTTGG |
| | | | TAGCTCTTGATCCGGCAAACAAACCACCGCTGGTAGCGGT |
| | | | GGTTTTTTTGTTTGCAAGCAGCAGATTACGCGCAGAAAAA |
| | | | AAGGATCTCAAGAAGATCCTTTGATCTTTTCTACGGGGTC |
| | | | TGACGCTCAGTGGAACGAAAACTCACGTTAAGGGATTTTG |
| | | | GTCATGAGATTATCAAAAAGGATCTTCACCTAGATCCTTT |
| | | | TAAATTAAAAATGAAGTTTTAAATCAATCTAAAGTATATA |
| | | | TGAGTAAACTTGGTCTGACAGTTAGAAAAACTCATCGAGC |
| | | | ATCAAATGAAACTGCAATTTATTCATATCAGGATTATCAA |
| | | | TACCATATTTTTGAAAAAGCCGTTTCTGTAATGAAGGAGA |
| | | | AAACTCACCGAGGCAGTTCCATAGGATGGCAAGATCCTGG |
| | | | TATCGGTCTGCGATTCCGACTCGTCCAACATCAATACAACC |
| | | | TATTAATTTCCCCTCGTCAAAAATAAGGTTATCAAGTGAG |
| | | | AAATCACCATGAGTGACGACTGAATCCGGTGAGAATGGCA |
| | | | AAAGTTTATGCATTTCTTTCCAGACTTGTTCAACAGGCCA |
| | | | GCCATTACGCTCGTCATCAAAATCACTCGCATCAACCAAAC |
| | | | CGTTATTCATTCGTGATTGCGCCTGAGCGAGACGAAATAC |
| | | | GCGATCGCTGTTAAAAGGACAATTACAAACAGGAATCGAA |
| | | | TGCAACCGGCGCAGGAACACTGCCAGCGCATCAACAATAT |
| | | | TTTCACCTGAATCAGGATATTCTTCTAATACCTGGAATGC |
| | | | TGTTTTCCCAGGGATCGCAGTGGTGAGTAACCATGCATCA |
| | | | TCAGGAGTACGGATAAAATGCTTGATGGTCGGAAGAGGCA |
| | | | TAAATTCCGTCAGCCAGTTTAGTCTGACCATCTCATCTGT |
| | | | AACATCATTGGCAACGCTACCTTTGCCATGTTTCAGAAAC |
| | | | AACTCTGGCGCATCGGGCTTCCCATACAATCGATAGATTG |
| | | | TCGCACCTGATTGCCCGACATTATCGCGAGCCCATTTATAC |
| | | | CCATATAAATCAGCATCCATGTTGGAATTTAATCGCGGCC |
| | | | TAGAGCAAGACGTTTCCCGTTGAATATGGCTCATACTCTT |
| | | | CCTTTTTCAATATTATTGAAGCATTTATCAGGGTTATTGT |
| | | | CTCATGAGCGGATACATATTTGAATGTATTTAGAAAAATA |
| | | | AACAAATAGGGGTTCCGCGCACATTTCCCCGAAAAGTGCC |
| | | | ACCTGACGTCTAAGAAACCATTATTATCATGACATTAACC |
| | | | TATAAAAATAGGCGTATCACGAGGCCCTTTCGTC |
| 64 | pYZZ454 | p5-R2C2-p5p5 | TCGCGCGTTTCGGTGATGACGGTGAAAACCTCTGA |
| | | | CACATGCAGCTCCCGGAGACGGTCACAGCTTGTCT |
| | | | GTAAGCGGATGCCGGGAGCAGACAAGCCCGTCAG |
| | | | GGCGCGTCAGCGGGTGTTGGCGGGTGTCGGGGCTG |
| | | | GCTTAACTATGCGGCATCAGAGCAGATTGTACTGA |
| | | | GAGTGCACCATATGCGGTGTGAAATACCGCACAGA |
| | | | TGCGTAAGGAGAAAATACCGCATCAGGCGCCATTC |
| | | | GCCATTCAGGCTGCGCAACTGTTGGGAAGGGCGAT |
| | | | CGGTGCGGGCCTCTTCGCTATTACGCCAGCTGGCG |
| | | | AAAGGGGGATGTGCTGCAAGGCGATTAAGTTGGGT |
| | | | AACGCCAGGGTTTTCCCAGTCACGACGTTGTAAAA |
| | | | CGACGGCCAGTGAATTGACGCGTATTGGGATGAGG |
| | | | TCCTGTATTAGAGGTCACGTGAGTGTTTTGCGACAT |
| | | | TTTGCGACACCATGTGGTCACGCTGGGTATTTAAGC |
| | | | CCGAGTGAGCACGCAGGGTCTCCATTTTGAAGCGG |
| | | | GAGGTTTGAACGCGCAGCCGCCATGCCGGGGTTTT |
| | | | ACGAGATTGTGATTAAGGTCCCCAGCGACCTTGAC |
| | | | GAGCATCTGCCCGGCATTTCTGACAGCTTTGTGAAC |
| | | | TGGGTGGCCGAGAAGGAATGGGAGTTGCCGCCAG |
| | | | ATTCTGACATGGATCTGAATCTGATTGAGCAGGCA |
| | | | CCCCTGACCGTGGCCGAGAAGCTGCAGCGCGACTT |
| | | | TCTGACGGAATGGCGCCGTGTGAGTAAGGCCCCGG |
| | | | AGGCTCTTTTCTTTGTGCAATTTGAGAAGGGAGAG |
| | | | AGCTACTTCCACATGCACGTGCTCGTGGAAACCAC |
| | | | CGGGGTGAAATCCATGGTTTTGGGACGTTTCCTGA |
| | | | GTCAGATTCGCGAAAAACTGATTCAGAGAATTTAC |
| | | | CGCGGGATCGAGCCGACTTTGCCAAACTGGTTCGC |
| | | | GGTCACAAAGACCAGAAATGGCGCCGGAGGCGGG |
| | | | AACAAGGTGGTGGATGAGTGCTACATCCCCAATTA |
| | | | CTTGCTCCCCAAAACCCAGCCTGAGCTCCAGTGGG |
| | | | CGTGGACTAATATGGAACAGTATTTAAGCGCCTGT |
| | | | TTGAATCTCACGGAGCGTAAACGGTTGGTGGCGCA |
| | | | GCATCTGACGCACGTGTCGCAGACGCAGGAGCAGA |
| | | | ACAAAGAGAATCAGAATCCCAATTCTGATGCGCCG |
| | | | GTGATCAGATCAAAAACTTCAGCCAGGTACATGGA |
| | | | GCTGGTCGGGTGGCTCGTGGACAAGGGGATTACCT |
| | | | CGGAGAAGCAGTGGATCCAGGAGGACCAGGCCTC |
| | | | ATACATCTCCTTCAATGCGGCCTCCAACTCGCGGTC |
| | | | CCAAATCAAGGCTGCCTTGGACAATGCGGGAAAGA |
| | | | TTATGAGCCTGACTAAAACCGCCCCCGACTACCTG |
| | | | GTGGGCCAGCAGCCCGTGGAGGACATTTCCAGCAA |
| | | | TCGGATTTATAAAATTTTGGAACTAAACGGGTACG |
| | | | ATCCCCAATATGCGGCTTCCGTCTTTCTGGGATGGG |
| | | | CCACGAAAAAGTTCGGCAAGAGGAACACCATCTGG |
| | | | CTGTTTGGGCCTGCAACTACCGGGAAGACCAACAT |
| | | | CGCGGAGGCCATAGCCCACACTGTGCCCTTCTACG |
| | | | GGTGCGTAAACTGGACCAATGAGAACTTTCCCTTC |
| | | | AACGACTGTGTCGACAAGATGGTGATCTGGTGGGA |
| | | | GGAGGGGAAGATGACCGCCAAGGTCGTGGAGTCG |
| | | | GCCAAAGCCATTCTCGGAGGAAGCAAGGTGCGCGT |
| | | | GGACCAGAAATGCAAGTCCTCGGCCCAGATAGACC |
| | | | CGACTCCCGTGATCGTCACCTCCAACACCAACATG |
| | | | TGCGCCGTGATTGACGGGAACTCAACGACCTTCGA |
| | | | ACACCAGCAGCCGTTGCAAGACCGGATGTTCAAAT |
| | | | TTGAACTCACCCGCCGTCTGGATCATGACTTTGGGA |
| | | | AGGTCACCAAGCAGGAAGTCAAAGACTTTTTCCGG |
| | | | TGGGCAAAGGATCACGTGGTTGAGGTGGAGCATGA |
| | | | ATTCTACGTCAAAAAGGGTGGAGCCAAGAAAAGA |
| | | | CCCGCCCCCAGTGACGCAGATATAAGTGAGCCCAA |
| | | | ACGGGTGCGCGAGTCAGTTGCGCAGCCATCGACGT |
| | | | CAGACGCGGAAGCTTCGATCAACTACGCAGACAGG |
| | | | TACCAAAACAAATGTTCTCGTCACGTGGGCATGAA |
| | | | TCTGATGCTGTTTCCCTGCAGACAATGCGAGAGAA |
| | | | TGAATCAGAATTCAAATATCTGCTTCACTCACGGA |
| | | | CAGAAAGACTGTTTAGAGTGCTTTCCCGTGTCAGA |
| | | | ATCTCAACCCGTTTCTGTCGTCAAAAAGGCGTATCA |
| | | | GAAACTGTGCTACATTCATCATATCATGGGAAAGG |
| | | | TGCCAGACGCTTGCACTGCCTGCGATCTGGTCAAT |
| | | | GTGGATTTGGATGACTGCATCTTTGAACAATAAAT |
| | | | GATTTAAATCAGGTatggctgccgatggttatcttccagattggctcgag |
| | | | gacactctctctgaaggaataagacagtggtggaagctcaaacctggcccaccacc |
| | | | accaaagcccgcagagcggcataaggacgacagcaggggtcttgtgcttcctggg |
| | | | tacaagtacctcggacccttcaacggactcgacaagggagagccggtcaacgagg |
| | | | cagacgccgcggccctcgagcacgacaaagcctacgaccggcagctcgacagcg |
| | | | gagacaacccgtacctcaagtacaaccacgccgacgcggagtttcaggagcgcctt |
| | | | aaagaagatacgtcttttgggggcaacctcggacgagcagtcttccaggcgaaaaa |
| | | | gagggttcttgaacctctgggcctggttgaggaacctgttaagacggctccgggaaa |
| | | | aaagaggccggtagagcactctcctgtggagccagactcctcctcgggaaccgga |
| | | | aaggcgggccagcagcctgcaagaaaaagattgaattttggtcagactggagacgc |
| | | | agactcagtacctgacccccagcctctcggacagccaccagcagccccctctggtc |
| | | | tgggaactaatacgatggctacaggcagtggcgcaccaatggcagacaataacga |
| | | | gggcgccgacggagtgggtaattcctcgggaaattggcattgcgattccacatggat |
| | | | gggcgacagagtcatcaccaccagcacccgaacctgggccctgcccacctacaac |
| | | | aaccacctctacaaacaaatttccagccaatcaggagcctcgaacgacaatcactac |
| | | | tttggctacagcaccccttgggggtattttgacttcaacagattccactgccacttttcac |
| | | | cacgtgactggcaaagactcatcaacaacaactggggattccgacccaagagactc |
| | | | aacttcaagctctttaacattcaagtcaaagaggtcacgcagaatgacggtacgacg |
| | | | acgattgccaataaccttaccagcacggttcaggtgtttactgactcggagtaccagc |
| | | | tcccgtacgtcctcggctcggcgcatcaaggatgcctcccgccgttcccagcagac |
| | | | gtcttcatggtgccacagtatggatacctcaccctgaacaacgggagtcaggcagta |
| | | | ggacgctcttcattttactgcctggagtactttccttctcagatgctgcgtaccggaaac |
| | | | aactttaccttcagctacacttttgaggacgttcctttccacagcagctacgctcacagc |
| | | | cagagtctggaccgtctcatgaatcctctcatcgaccagtacctgtattacttgagcag |
| | | | aacaaacactccaagtggaaccaccacgcagtcaaggcttcagttttctcaggccgg |
| | | | agcgagtgacattcgggaccagtctaggaactggcttcctggaccctgttaccgcca |
| | | | gcagcgagtatcaaagacatctgcggataacaacaacagtgaatactcgtggactg |
| | | | gagctaccaagtaccacctcaatggcagagactctctggtgaatccgggcccggcc |
| | | | atggcaagccacaaggacgatgaagaaaagttttttcctcagagcggggttctcatct |
| | | | ttgggaagcaaggctcagagaaaacaaatgtggacattgaaaaggtcatgattacag |
| | | | acgaagaggaaatcaggacaaccaatcccgtggctacggagcagtatggttctgtat |
| | | | ctaccaacctccagagaggcaacagacaagcagctaccgcagatgtcaacacaca |
| | | | aggcgttcttccaggcatggtctggcaggacagagatgtgtaccttcaggggcccat |
| | | | ctgggcaaagattccacacacggacggacattttcacccctctcccctcatggggg |
| | | | attcggacttaaacaccctcctccacagattctcatcaagaacaccccggtacctgcg |
| | | | aatccttcgaccaccttcagtgcggcaaagtttgcttccttcatcacacagtactccac |
| | | | gggacaggtcagcgtggagatcgagtgggagctgcagaaggaaaacagcaaacg |
| | | | ctggaatcccgaaattcagtacacttccaactacaacaagtctgttaatgtggactttac |
| | | | tgtggacactaatggcgtgtattcagagcctcgccccattggcaccagatacctgact |
| | | | cgtaatctgtaattgcttgttaatcaataaaccgtttaattcgtttcagttgaactttggt |
| | | | ctctgcgaagggcgaattcgtttaaacctgcaggactagaggtcctgtattagaggtcac |
| | | | gtgagtgttttgcgacattttgcgacaccatgtggtcacgctgggtatttaagcccgag |
| | | | tgagcacgcagggtctccattttgaagcgggaggtttgaacgcgcagccgccaagc |
| | | | cgaattctgcagatccggttaattcgtttcagttgaactttggtctcctgtccttcttatc |
| | | | ttatcggttaccatagaaactggttacttattaactgcttggtgcgcttcgcgataaaaga |
| | | | cttacgtcatcgggttacccctagtgatggagcggccgctttcagttgaactttggtctct |
| | | | gcgtatttctttcttatctagtttccatgctctagaggtcctgtattagaggtcacgtgagt |
| | | | gttttgcgacattttgcgacaccatgtggtcacgctgggtatttaagcccgagtgagca |
| | | | cgcagggtctccattttgaaggggaggtttgaacgcgcagccgccaagccgaatt |
| | | | ctgcagaatcccaatGGCGCGCCGAGCTTGGCTCGAGCATG |
| | | | GTCATAGCTGTTTCCTGTGTGAAATTGTTATCCGCT |
| | | | CACAATTCCACACAACATACGAGCCGGAAGCATAA |
| | | | AGTGTAAAGCCTGGGGTGCCTAATGAGTGAGCTAA |
| | | | CTCACATTAATTGCGTTGCGCTCACTGCCCGCTTTC |
| | | | CAGTCGGGAAACCTGTCGTGCCAGCTGCATTAATG |
| | | | AATCGGCCAACGCGCGGGGAGAGGCGGTTTGCGTA |
| | | | TTGGGCGCTCTTCCGCTTCCTCGCTCACTGACTCGC |
| | | | TGCGCTCGGTCGTTCGGCTGCGGCGAGCGGTATCA |
| | | | GCTCACTCAAAGGCGGTAATACGGTTATCCACAGA |
| | | | ATCAGGGGATAACGCAGGAAAGAACATGTGAGCA |
| | | | AAAGGCCAGCAAAAGGCCAGGAACCGTAAAAAGG |
| | | | CCGCGTTGCTGGCGTTTTTCCATAGGCTCCGCCCCC |
| | | | CTGACGAGCATCACAAAAATCGACGCTCAAGTCAG |
| | | | AGGTGGCGAAACCCGACAGGACTATAAAGATACC |
| | | | AGGCGTTTCCCCCTGGAAGCTCCCTCGTGCGCTCTC |
| | | | CTGTTCCGACCCTGCCGCTTACCGGATACCTGTCCG |
| | | | CCTTTCTCCCTTCGGGAAGCGTGGCGCTTTCTCATA |
| | | | GCTCACGCTGTAGGTATCTCAGTTCGGTGTAGGTCG |
| | | | TTCGCTCCAAGCTGGGCTGTGTGCACGAACCCCCC |
| | | | GTTCAGCCCGACCGCTGCGCCTTATCCGGTAACTAT |
| | | | CGTCTTGAGTCCAACCCGGTAAGACACGACTTATC |
| | | | GCCACTGGCAGCAGCCACTGGTAACAGGATTAGCA |
| | | | GAGCGAGGTATGTAGGCGGTGCTACAGAGTTCTTG |
| | | | AAGTGGTGGCCTAACTACGGCTACACTAGAAGAAC |
| | | | AGTATTTGGTATCTGCGCTCTGCTGAAGCCAGTTAC |
| | | | CTTCGGAAAAAGAGTTGGTAGCTCTTGATCCGGCA |
| | | | AACAAACCACCGCTGGTAGCGGTGGTTTTTTTGTTT |
| | | | GCAAGCAGCAGATTACGCGCAGAAAAAAAGGATC |
| | | | TCAAGAAGATCCTTTGATCTTTTCTACGGGGTCTGA |
| | | | CGCTCAGTGGAACGAAAACTCACGTTAAGGGATTT |
| | | | TGGTCATGAGATTATCAAAAAGGATCTTCACCTAG |
| | | | ATCCTTTTAAATTAAAAATGAAGTTTTAAATCAATC |
| | | | TAAAGTATATATGAGTAAACTTGGTCTGACAGTTA |
| | | | GAAAAACTCATCGAGCATCAAATGAAACTGCAATT |
| | | | TATTCATATCAGGATTATCAATACCATATTTTTGAA |
| | | | AAAGCCGTTTCTGTAATGAAGGAGAAAACTCACCG |
| | | | AGGCAGTTCCATAGGATGGCAAGATCCTGGTATCG |
| | | | GTCTGCGATTCCGACTCGTCCAACATCAATACAAC |
| | | | CTATTAATTTCCCCTCGTCAAAAATAAGGTTATCAA |
| | | | GTGAGAAATCACCATGAGTGACGACTGAATCCGGT |
| | | | GAGAATGGCAAAAGTTTATGCATTTCTTTCCAGACT |
| | | | TGTTCAACAGGCCAGCCATTACGCTCGTCATCAAA |
| | | | ATCACTCGCATCAACCAAACCGTTATTCATTCGTGA |
| | | | TTGCGCCTGAGCGAGACGAAATACGCGATCGCTGT |
| | | | TAAAAGGACAATTACAAACAGGAATCGAATGCAA |
| | | | CCGGCGCAGGAACACTGCCAGCGCATCAACAATAT |
| | | | TTTCACCTGAATCAGGATATTCTTCTAATACCTGGA |
| | | | ATGCTGTTTTCCCAGGGATCGCAGTGGTGAGTAAC |
| | | | CATGCATCATCAGGAGTACGGATAAAATGCTTGAT |
| | | | GGTCGGAAGAGGCATAAATTCCGTCAGCCAGTTTA |
| | | | GTCTGACCATCTCATCTGTAACATCATTGGCAACGC |
| | | | TACCTTTGCCATGTTTCAGAAACAACTCTGGCGCAT |
| | | | CGGGCTTCCCATACAATCGATAGATTGTCGCACCT |
| | | | GATTGCCCGACATTATCGCGAGCCCATTTATACCCA |
| | | | TATAAATCAGCATCCATGTTGGAATTTAATCGCGG |
| | | | CCTAGAGCAAGACGTTTCCCGTTGAATATGGCTCA |
| | | | TACTCTTCCTTTTTCAATATTATTGAAGCATTTATCA |
| | | | GGGTTATTGTCTCATGAGCGGATACATATTTGAATG |
| | | | TATTTAGAAAAATAAACAAATAGGGGTTCCGCGCA |
| | | | CATTTCCCCGAAAAGTGCCACCTGACGTCTAAGAA |
| | | | ACCATTATTATCATGACATTAACCTATAAAAATAG |
| | | | GCGTATCACGAGGCCCTTTCGTC |
| 65 | pYZZ455 | p5- | TCGCGCGTTTCGGTGATGACGGTGAAAACCTCTGA |
| | | AltRep2Cap2- | CACATGCAGCTCCCGGAGACGGTCACAGCTTGTCT |
| | | p5p5 | GTAAGCGGATGCCGGGAGCAGACAAGCCCGTCAG |
| | | | GGCGCGTCAGCGGGTGTTGGCGGGTGTCGGGGCTG |
| | | | GCTTAACTATGCGGCATCAGAGCAGATTGTACTGA |
| | | | GAGTGCACCATATGCGGTGTGAAATACCGCACAGA |
| | | | TGCGTAAGGAGAAAATACCGCATCAGGCGCCATTC |
| | | | GCCATTCAGGCTGCGCAACTGTTGGGAAGGGCGAT |
| | | | CGGTGCGGGCCTCTTCGCTATTACGCCAGCTGGCG |
| | | | AAAGGGGGATGTGCTGCAAGGCGATTAAGTTGGGT |
| | | | AACGCCAGGGTTTTCCCAGTCACGACGTTGTAAAA |
| | | | CGACGGCCAGTGAATTGACGCGTATTGGGATGAGG |
| | | | TCCTGTATTAGAGGTCACGTGAGTGTTTTGCGACAT |
| | | | TTTGCGACACCATGTGGTCACGCTGGGTATTTAAGC |
| | | | CCGAGTGAGCACGCAGGGTCTCCATTTTGAAGCGG |
| | | | GAGGTTTGAACGCGCAGCCGCCACGCCGGGGTTTT |
| | | | ACGAGATTGTGATTAAGGTCCCCAGCGACCTTGAC |
| | | | GAGCATCTGCCCGGCATTTCTGACAGCTTTGTGAAC |
| | | | TGGGTGGCCGAGAAGGAATGGGAGTTGCCGCCAG |
| | | | ATTCTGACATGGATCTGAATCTGATTGAGCAGGCA |
| | | | CCCCTGACCGTGGCCGAGAAGCTGCAGCGCGACTT |
| | | | TCTGACGGAATGGCGCCGTGTGAGTAAGGCCCCGG |
| | | | AGGCTCTTTTCTTTGTGCAATTTGAGAAGGGAGAG |
| | | | AGCTACTTCCACATGCACGTGCTCGTGGAAACCAC |
| | | | CGGGGTGAAATCCATGGTTTTGGGACGTTTCCTGA |
| | | | GTCAGATTCGCGAAAAACTGATTCAGAGAATTTAC |
| | | | CGCGGGATCGAGCCGACTTTGCCAAACTGGTTCGC |
| | | | GGTCACAAAGACCAGAAATGGCGCCGGAGGCGGG |
| | | | AACAAGGTGGTGGATGAGTGCTACATCCCCAATTA |
| | | | CTTGCTCCCCAAAACCCAGCCTGAGCTCCAGTGGG |
| | | | CGTGGACTAATATGGAACAGTATTTAAGCGCCTGT |
| | | | TTGAATCTCACGGAGCGTAAACGGTTGGTGGCGCA |
| | | | GCATCTGACGCACGTGTCGCAGACGCAGGAGCAGA |
| | | | ACAAAGAGAATCAGAATCCCAATTCTGATGCGCCG |
| | | | GTGATCAGATCAAAAACTTCAGCCAGGTACATGGA |
| | | | GCTGGTCGGGTGGCTCGTGGACAAGGGGATTACCT |
| | | | CGGAGAAGCAGTGGATCCAGGAGGACCAGGCCTC |
| | | | ATACATCTCCTTCAATGCGGCCTCCAACTCGCGGTC |
| | | | CCAAATCAAGGCTGCCTTGGACAATGCGGGAAAGA |
| | | | TTATGAGCCTGACTAAAACCGCCCCCGACTACCTG |
| | | | GTGGGCCAGCAGCCCGTGGAGGACATTTCCAGCAA |
| | | | TCGGATTTATAAAATTTTGGAACTAAACGGGTACG |
| | | | ATCCCCAATATGCGGCTTCCGTCTTTCTGGGATGGG |
| | | | CCACGAAAAAGTTCGGCAAGAGGAACACCATCTGG |
| | | | CTGTTTGGGCCTGCAACTACCGGGAAGACCAACAT |
| | | | CGCGGAGGCCATAGCCCACACTGTGCCCTTCTACG |
| | | | GGTGCGTAAACTGGACCAATGAGAACTTTCCCTTC |
| | | | AACGACTGTGTCGACAAGATGGTGATCTGGTGGGA |
| | | | GGAGGGGAAGATGACCGCCAAGGTCGTGGAGTCG |
| | | | GCCAAAGCCATTCTCGGAGGAAGCAAGGTGCGCGT |
| | | | GGACCAGAAATGCAAGTCCTCGGCCCAGATAGACC |
| | | | CGACTCCCGTGATCGTCACCTCCAACACCAACATG |
| | | | TGCGCCGTGATTGACGGGAACTCAACGACCTTCGA |
| | | | ACACCAGCAGCCGTTGCAAGACCGGATGTTCAAAT |
| | | | TTGAACTCACCCGCCGTCTGGATCATGACTTTGGGA |
| | | | AGGTCACCAAGCAGGAAGTCAAAGACTTTTTCCGG |
| | | | TGGGCAAAGGATCACGTGGTTGAGGTGGAGCATGA |
| | | | ATTCTACGTCAAAAAGGGTGGAGCCAAGAAAAGA |
| | | | CCCGCCCCCAGTGACGCAGATATAAGTGAGCCCAA |
| | | | ACGGGTGCGCGAGTCAGTTGCGCAGCCATCGACGT |
| | | | CAGACGCGGAAGCTTCGATCAACTACGCAGACAGG |
| | | | TACCAAAACAAATGTTCTCGTCACGTGGGCATGAA |
| | | | TCTGATGCTGTTTCCCTGCAGACAATGCGAGAGAA |
| | | | TGAATCAGAATTCAAATATCTGCTTCACTCACGGA |
| | | | CAGAAAGACTGTTTAGAGTGCTTTCCCGTGTCAGA |
| | | | ATCTCAACCCGTTTCTGTCGTCAAAAAGGCGTATCA |
| | | | GAAACTGTGCTACATTCATCATATCATGGGAAAGG |
| | | | TGCCAGACGCTTGCACTGCCTGCGATCTGGTCAAT |
| | | | GTGGATTTGGATGACTGCATCTTTGAACAATAAAT |
| | | | GATTTAAATCAGGTatggctgccgatggttatcttccagattggctcgag |
| | | | gacactctctctgaaggaataagacagtggtggaagctcaaacctggcccaccacc |
| | | | accaaagcccgcagagcggcataaggacgacagcaggggtcttgtgcttcctggg |
| | | | tacaagtacctcggacccttcaacggactcgacaagggagagccggtcaacgagg |
| | | | cagacgccgcggccctcgagcacgacaaagcctacgaccggcagctcgacagcg |
| | | | gagacaacccgtacctcaagtacaaccacgccgacgcggagtttcaggagcgcctt |
| | | | aaagaagatacgtcttttgggggcaacctcggacgagcagtcttccaggcgaaaaa |
| | | | gagggttcttgaacctctgggcctggttgaggaacctgttaagacggctccgggaaa |
| | | | aaagaggccggtagagcactctcctgtggagccagactcctcctcgggaaccgga |
| | | | aaggcgggccagcagcctgcaagaaaaagattgaattttggtcagactggagacgc |
| | | | agactcagtacctgacccccagcctctcggacagccaccagcagccccctctggtc |
| | | | tgggaactaatacgatggctacaggcagtggcgcaccaatggcagacaataacga |
| | | | gggcgccgacggagtgggtaattcctcgggaaattggcattgcgattccacatggat |
| | | | gggcgacagagtcatcaccaccagcacccgaacctgggccctgcccacctacaac |
| | | | aaccacctctacaaacaaatttccagccaatcaggagcctcgaacgacaatcactac |
| | | | tttggctacagcaccccttgggggtattttgacttcaacagattccactgccacttttcac |
| | | | cacgtgactggcaaagactcatcaacaacaactggggattccgacccaagagactc |
| | | | aacttcaagctctttaacattcaagtcaaagaggtcacgcagaatgacggtacgacg |
| | | | acgattgccaataaccttaccagcacggttcaggtgtttactgactcggagtaccagc |
| | | | tcccgtacgtcctcggctcggcgcatcaaggatgcctcccgccgttcccagcagac |
| | | | gtcttcatggtgccacagtatggatacctcaccctgaacaacgggagtcaggcagta |
| | | | ggacgctcttcattttactgcctggagtactttccttctcagatgctgcgtaccggaaac |
| | | | aactttaccttcagctacacttttgaggacgttcctttccacagcagctacgctcacagc |
| | | | cagagtctggaccgtctcatgaatcctctcatcgaccagtacctgtattacttgagcag |
| | | | aacaaacactccaagtggaaccaccacgcagtcaaggcttcagttttctcaggccgg |
| | | | agcgagtgacattcgggaccagtctaggaactggcttcctggaccctgttaccgcca |
| | | | gcagcgagtatcaaagacatctgcggataacaacaacagtgaatactcgtggactg |
| | | | gagctaccaagtaccacctcaatggcagagactctctggtgaatccgggcccggcc |
| | | | atggcaagccacaaggacgatgaagaaaagttttttcctcagagcggggttctcatct |
| | | | ttgggaagcaaggctcagagaaaacaaatgtggacattgaaaaggtcatgattacag |
| | | | acgaagaggaaatcaggacaaccaatcccgtggctacggagcagtatggttctgtat |
| | | | ctaccaacctccagagaggcaacagacaagcagctaccgcagatgtcaacacaca |
| | | | aggcgttcttccaggcatggtctggcaggacagagatgtgtaccttcaggggcccat |
| | | | ctgggcaaagattccacacacggacggacattttcacccctctcccctcatgggtgg |
| | | | attcggacttaaacaccctcctccacagattctcatcaagaacaccccggtacctgcg |
| | | | aatccttcgaccaccttcagtgcggcaaagtttgcttccttcatcacacagtactccac |
| | | | gggacaggtcagcgtggagatcgagtgggagctgcagaaggaaaacagcaaacg |
| | | | ctggaatcccgaaattcagtacacttccaactacaacaagtctgttaatgtggactttac |
| | | | tgtggacactaatggcgtgtattcagagcctcgccccattggcaccagatacctgact |
| | | | cgtaatctgtaattgcttgttaatcaataaaccgtttaattcgtttcagttgaactttggt |
| | | | ctctgcgaagggcgaattcgtttaaacctgcaggactagaggtcctgtattagaggtcac |
| | | | gtgagtgttttgcgacattttgcgacaccatgtggtcacgctgggtatttaagcccgag |
| | | | tgagcacgcagggtctccattttgaagcgggaggtttgaacgcgcagccgccaagc |
| | | | cgaattctgcagatccggttaattcgtttcagttgaactttggtctcctgtccttcttatc |
| | | | ttatcggttaccatagaaactggttacttattaactgcttggtgcgcttcgcgataaaaga |
| | | | cttacgtcatcgggttacccctagtgatggagcggccgctttcagttgaactttggtctct |
| | | | gcgtatttctttcttatctagtttccatgctctagaggtcctgtattagaggtcacgtgag |
| | | | tgttttgcgacattttgcgacaccatgtggtcacgctgggtatttaagcccgagtgagca |
| | | | cgcagggtctccattttgaagcgggaggtttgaacgcgcagccgccaagccgaatt |
| | | | ctgcagaatcccaatGGCGCGCCGAGCTTGGCTCGAGCATG |
| | | | GTCATAGCTGTTTCCTGTGTGAAATTGTTATCCGCT |
| | | | CACAATTCCACACAACATACGAGCCGGAAGCATAA |
| | | | AGTGTAAAGCCTGGGGTGCCTAATGAGTGAGCTAA |
| | | | CTCACATTAATTGCGTTGCGCTCACTGCCCGCTTTC |
| | | | CAGTCGGGAAACCTGTCGTGCCAGCTGCATTAATG |
| | | | AATCGGCCAACGCGCGGGGAGAGGCGGTTTGCGTA |
| | | | TTGGGCGCTCTTCCGCTTCCTCGCTCACTGACTCGC |
| | | | TGCGCTCGGTCGTTCGGCTGCGGCGAGCGGTATCA |
| | | | GCTCACTCAAAGGCGGTAATACGGTTATCCACAGA |
| | | | ATCAGGGGATAACGCAGGAAAGAACATGTGAGCA |
| | | | AAAGGCCAGCAAAAGGCCAGGAACCGTAAAAAGG |
| | | | CCGCGTTGCTGGCGTTTTTCCATAGGCTCCGCCCCC |
| | | | CTGACGAGCATCACAAAAATCGACGCTCAAGTCAG |
| | | | AGGTGGCGAAACCCGACAGGACTATAAAGATACC |
| | | | AGGCGTTTCCCCCTGGAAGCTCCCTCGTGCGCTCTC |
| | | | CTGTTCCGACCCTGCCGCTTACCGGATACCTGTCCG |
| | | | CCTTTCTCCCTTCGGGAAGCGTGGCGCTTTCTCATA |
| | | | GCTCACGCTGTAGGTATCTCAGTTCGGTGTAGGTCG |
| | | | TTCGCTCCAAGCTGGGCTGTGTGCACGAACCCCCC |
| | | | GTTCAGCCCGACCGCTGCGCCTTATCCGGTAACTAT |
| | | | CGTCTTGAGTCCAACCCGGTAAGACACGACTTATC |
| | | | GCCACTGGCAGCAGCCACTGGTAACAGGATTAGCA |
| | | | GAGCGAGGTATGTAGGCGGTGCTACAGAGTTCTTG |
| | | | AAGTGGTGGCCTAACTACGGCTACACTAGAAGAAC |
| | | | AGTATTTGGTATCTGCGCTCTGCTGAAGCCAGTTAC |
| | | | CTTCGGAAAAAGAGTTGGTAGCTCTTGATCCGGCA |
| | | | AACAAACCACCGCTGGTAGCGGTGGTTTTTTTGTTT |
| | | | GCAAGCAGCAGATTACGCGCAGAAAAAAAGGATC |
| | | | TCAAGAAGATCCTTTGATCTTTTCTACGGGGTCTGA |
| | | | CGCTCAGTGGAACGAAAACTCACGTTAAGGGATTT |
| | | | TGGTCATGAGATTATCAAAAAGGATCTTCACCTAG |
| | | | ATCCTTTTAAATTAAAAATGAAGTTTTAAATCAATC |
| | | | TAAAGTATATATGAGTAAACTTGGTCTGACAGTTA |
| | | | GAAAAACTCATCGAGCATCAAATGAAACTGCAATT |
| | | | TATTCATATCAGGATTATCAATACCATATTTTTGAA |
| | | | AAAGCCGTTTCTGTAATGAAGGAGAAAACTCACCG |
| | | | AGGCAGTTCCATAGGATGGCAAGATCCTGGTATCG |
| | | | GTCTGCGATTCCGACTCGTCCAACATCAATACAAC |
| | | | CTATTAATTTCCCCTCGTCAAAAATAAGGTTATCAA |
| | | | GTGAGAAATCACCATGAGTGACGACTGAATCCGGT |
| | | | GAGAATGGCAAAAGTTTATGCATTTCTTTCCAGACT |
| | | | TGTTCAACAGGCCAGCCATTACGCTCGTCATCAAA |
| | | | ATCACTCGCATCAACCAAACCGTTATTCATTCGTGA |
| | | | TTGCGCCTGAGCGAGACGAAATACGCGATCGCTGT |
| | | | TAAAAGGACAATTACAAACAGGAATCGAATGCAA |
| | | | CCGGCGCAGGAACACTGCCAGCGCATCAACAATAT |
| | | | TTTCACCTGAATCAGGATATTCTTCTAATACCTGGA |
| | | | ATGCTGTTTTCCCAGGGATCGCAGTGGTGAGTAAC |
| | | | CATGCATCATCAGGAGTACGGATAAAATGCTTGAT |
| | | | GGTCGGAAGAGGCATAAATTCCGTCAGCCAGTTTA |
| | | | GTCTGACCATCTCATCTGTAACATCATTGGCAACGC |
| | | | TACCTTTGCCATGTTTCAGAAACAACTCTGGCGCAT |
| | | | CGGGCTTCCCATACAATCGATAGATTGTCGCACCT |
| | | | GATTGCCCGACATTATCGCGAGCCCATTTATACCCA |
| | | | TATAAATCAGCATCCATGTTGGAATTTAATCGCGG |
| | | | CCTAGAGCAAGACGTTTCCCGTTGAATATGGCTCA |
| | | | TACTCTTCCTTTTTCAATATTATTGAAGCATTTATCA |
| | | | GGGTTATTGTCTCATGAGCGGATACATATTTGAATG |
| | | | TATTTAGAAAAATAAACAAATAGGGGTTCCGCGCA |
| | | | CATTTCCCCGAAAAGTGCCACCTGACGTCTAAGAA |
| | | | ACCATTATTATCATGACATTAACCTATAAAAATAG |
| | | | GCGTATCACGAGGCCCTTTCGTC |
| 66 | pYZZ405 | Rep2Cap2-p5p5 | TCGCGCGTTTCGGTGATGACGGTGAAAACCTCTGA |
| | | | CACATGCAGCTCCCGGAGACGGTCACAGCTTGTCT |
| | | | GTAAGCGGATGCCGGGAGCAGACAAGCCCGTCAG |
| | | | GGCGCGTCAGCGGGTGTTGGCGGGTGTCGGGGCTG |
| | | | GCTTAACTATGCGGCATCAGAGCAGATTGTACTGA |
| | | | GAGTGCACCATATGCGGTGTGAAATACCGCACAGA |
| | | | TGCGTAAGGAGAAAATACCGCATCAGGCGCCATTC |
| | | | GCCATTCAGGCTGCGCAACTGTTGGGAAGGGCGAT |
| | | | CGGTGCGGGCCTCTTCGCTATTACGCCAGCTGGCG |
| | | | AAAGGGGGATGTGCTGCAAGGCGATTAAGTTGGGT |
| | | | AACGCCAGGGTTTTCCCAGTCACGACGTTGTAAAA |
| | | | CGACGGCCAGTGAATTGACGCGTATTGGGATCGCG |
| | | | CAGCCGCCATGCCGGGGTTTTACGAGATTGTGATT |
| | | | AAGGTCCCCAGCGACCTTGACGAGCATCTGCCCGG |
| | | | CATTTCTGACAGCTTTGTGAACTGGGTGGCCGAGA |
| | | | AGGAATGGGAGTTGCCGCCAGATTCTGACATGGAT |
| | | | CTGAATCTGATTGAGCAGGCACCCCTGACCGTGGC |
| | | | CGAGAAGCTGCAGCGCGACTTTCTGACGGAATGGC |
| | | | GCCGTGTGAGTAAGGCCCCGGAGGCTCTTTTCTTTG |
| | | | TGCAATTTGAGAAGGGAGAGAGCTACTTCCACATG |
| | | | CACGTGCTCGTGGAAACCACCGGGGTGAAATCCAT |
| | | | GGTTTTGGGACGTTTCCTGAGTCAGATTCGCGAAA |
| | | | AACTGATTCAGAGAATTTACCGCGGGATCGAGCCG |
| | | | ACTTTGCCAAACTGGTTCGCGGTCACAAAGACCAG |
| | | | AAATGGCGCCGGAGGCGGGAACAAGGTGGTGGAT |
| | | | GAGTGCTACATCCCCAATTACTTGCTCCCCAAAACC |
| | | | CAGCCTGAGCTCCAGTGGGCGTGGACTAATATGGA |
| | | | ACAGTATTTAAGCGCCTGTTTGAATCTCACGGAGC |
| | | | GTAAACGGTTGGTGGCGCAGCATCTGACGCACGTG |
| | | | TCGCAGACGCAGGAGCAGAACAAAGAGAATCAGA |
| | | | ATCCCAATTCTGATGCGCCGGTGATCAGATCAAAA |
| | | | ACTTCAGCCAGGTACATGGAGCTGGTCGGGTGGCT |
| | | | CGTGGACAAGGGGATTACCTCGGAGAAGCAGTGG |
| | | | ATCCAGGAGGACCAGGCCTCATACATCTCCTTCAA |
| | | | TGCGGCCTCCAACTCGCGGTCCCAAATCAAGGCTG |
| | | | CCTTGGACAATGCGGGAAAGATTATGAGCCTGACT |
| | | | AAAACCGCCCCCGACTACCTGGTGGGCCAGCAGCC |
| | | | CGTGGAGGACATTTCCAGCAATCGGATTTATAAAA |
| | | | TTTTGGAACTAAACGGGTACGATCCCCAATATGCG |
| | | | GCTTCCGTCTTTCTGGGATGGGCCACGAAAAAGTT |
| | | | CGGCAAGAGGAACACCATCTGGCTGTTTGGGCCTG |
| | | | CAACTACCGGGAAGACCAACATCGCGGAGGCCATA |
| | | | GCCCACACTGTGCCCTTCTACGGGTGCGTAAACTG |
| | | | GACCAATGAGAACTTTCCCTTCAACGACTGTGTCG |
| | | | ACAAGATGGTGATCTGGTGGGAGGAGGGGAAGAT |
| | | | GACCGCCAAGGTCGTGGAGTCGGCCAAAGCCATTC |
| | | | TCGGAGGAAGCAAGGTGCGCGTGGACCAGAAATG |
| | | | CAAGTCCTCGGCCCAGATAGACCCGACTCCCGTGA |
| | | | TCGTCACCTCCAACACCAACATGTGCGCCGTGATT |
| | | | GACGGGAACTCAACGACCTTCGAACACCAGCAGCC |
| | | | GTTGCAAGACCGGATGTTCAAATTTGAACTCACCC |
| | | | GCCGTCTGGATCATGACTTTGGGAAGGTCACCAAG |
| | | | CAGGAAGTCAAAGACTTTTTCCGGTGGGCAAAGGA |
| | | | TCACGTGGTTGAGGTGGAGCATGAATTCTACGTCA |
| | | | AAAAGGGTGGAGCCAAGAAAAGACCCGCCCCCAG |
| | | | TGACGCAGATATAAGTGAGCCCAAACGGGTGCGCG |
| | | | AGTCAGTTGCGCAGCCATCGACGTCAGACGCGGAA |
| | | | GCTTCGATCAACTACGCAGACAGGTACCAAAACAA |
| | | | ATGTTCTCGTCACGTGGGCATGAATCTGATGCTGTT |
| | | | TCCCTGCAGACAATGCGAGAGAATGAATCAGAATT |
| | | | CAAATATCTGCTTCACTCACGGACAGAAAGACTGT |
| | | | TTAGAGTGCTTTCCCGTGTCAGAATCTCAACCCGTT |
| | | | TCTGTCGTCAAAAAGGCGTATCAGAAACTGTGCTA |
| | | | CATTCATCATATCATGGGAAAGGTGCCAGACGCTT |
| | | | GCACTGCCTGCGATCTGGTCAATGTGGATTTGGAT |
| | | | GACTGCATCTTTGAACAATAAATGATTTAAATCAG |
| | | | GTatggctgccgatggttatcttccagattggctcgaggacactctctctgaaggaat |
| | | | aagacagtggtggaagctcaaacctggcccaccaccaccaaagcccgcagagcg |
| | | | gcataaggacgacagcaggggtcttgtgcttcctgggtacaagtacctcggaccctt |
| | | | caacggactcgacaagggagagccggtcaacgaggcagacgccgcggccctcg |
| | | | agcacgacaaagcctacgaccggcagctcgacagcggagacaacccgtacctca |
| | | | agtacaaccacgccgacgcggagtttcaggagcgccttaaagaagatacgtcttttg |
| | | | ggggcaacctcggacgagcagtcttccaggcgaaaaagagggttcttgaacctctg |
| | | | ggcctggttgaggaacctgttaagacggctccgggaaaaaagaggccggtagagc |
| | | | actctcctgtggagccagactcctcctcgggaaccggaaagggggccagcagcc |
| | | | tgcaagaaaaagattgaattttggtcagactggagacgcagactcagtacctgaccc |
| | | | ccagcctctcggacagccaccagcagccccctctggtctgggaactaatacgatgg |
| | | | ctacaggcagtggcgcaccaatggcagacaataacgagggcgccgacggagtgg |
| | | | gtaattcctcgggaaattggcattgcgattccacatggatgggcgacagagtcatcac |
| | | | caccagcacccgaacctgggccctgcccacctacaacaaccacctctacaaacaaa |
| | | | tttccagccaatcaggagcctcgaacgacaatcactactttggctacagcaccccttg |
| | | | ggggtattttgacttcaacagattccactgccacttttcaccacgtgactggcaaagac |
| | | | tcatcaacaacaactggggattccgacccaagagactcaacttcaagctctttaacatt |
| | | | caagtcaaagaggtcacgcagaatgacggtacgacgacgattgccaataaccttac |
| | | | cagcacggttcaggtgtttactgactcggagtaccagctcccgtacgtcctcggctcg |
| | | | gcgcatcaaggatgcctcccgccgttcccagcagacgtcttcatggtgccacagtat |
| | | | ggatacctcaccctgaacaacgggagtcaggcagtaggacgctcttcattttactgcc |
| | | | tggagtactttccttctcagatgctgcgtaccggaaacaactttaccttcagctacacttt |
| | | | tgaggacgttcctttccacagcagctacgctcacagccagagtctggaccgtctcatg |
| | | | aatcctctcatcgaccagtacctgtattacttgagcagaacaaacactccaagtggaa |
| | | | ccaccacgcagtcaaggcttcagttttctcaggccggagcgagtgacattcgggacc |
| | | | agtctaggaactggcttcctggaccctgttaccgccagcagcgagtatcaaagacat |
| | | | ctgcggataacaacaacagtgaatactcgtggactggagctaccaagtaccacctca |
| | | | atggcagagactctctggtgaatccgggcccggccatggcaagccacaaggacga |
| | | | tgaagaaaagttttttcctcagagcggggttctcatctttgggaagcaaggctcagag |
| | | | aaaacaaatgtggacattgaaaaggtcatgattacagacgaagaggaaatcaggac |
| | | | aaccaatcccgtggctacggagcagtatggttctgtatctaccaacctccagagagg |
| | | | caacagacaagcagctaccgcagatgtcaacacacaaggcgttcttccaggcatgg |
| | | | tctggcaggacagagatgtgtaccttcaggggcccatctgggcaaagattccacaca |
| | | | cggacggacattttcacccctctcccctcatgggggattcggacttaaacaccctcct |
| | | | ccacagattctcatcaagaacaccccggtacctgcgaatccttcgaccaccttcagtg |
| | | | cggcaaagtttgcttccttcatcacacagtactccacgggacaggtcagcgtggaga |
| | | | tcgagtgggagctgcagaaggaaaacagcaaacgctggaatcccgaaattcagta |
| | | | cacttccaactacaacaagtctgttaatgtggactttactgtggacactaatggcgtgta |
| | | | ttcagagcctcgccccattggcaccagatacctgactcgtaatctgtaattgcttgttaa |
| | | | tcaataaaccgtttaattcgtttcagttgaactttggtctctgcgaagggcgaattcgttt |
| | | | aaacctgcaggactagaggtcctgtattagaggtcacgtgagtgttttgcgacattttg |
| | | | cgacaccatgtggtcacgctgggtatttaagcccgagtgagcacgcagggtctccat |
| | | | tttgaagcgggaggtttgaacgcgcagccgccaagccgaattctgcagatccggtta |
| | | | attcgtttcagttgaactttggtctcctgtccttcttatcttatcggttaccatagaaact |
| | | | ggttacttattaactgcttggtgcgcttcgcgataaaagacttacgtcatcgggttacccc |
| | | | tagtgatggagcggccgctttcagttgaactttggtctctgcgtatttctttcttatctag |
| | | | tttccatgctctagaggtcctgtattagaggtcacgtgagtgttttgcgacattttgcgac |
| | | | accatgtggtcacgctgggtatttaagcccgagtgagcacgcagggtctccattttgaag |
| | | | cgggaggtttgaacgcgcagccgccaagccgaattctgcagaatcccaatGGC |
| | | | GCGCCGAGCTTGGCTCGAGCATGGTCATAGCTGTT |
| | | | TCCTGTGTGAAATTGTTATCCGCTCACAATTCCACA |
| | | | CAACATACGAGCCGGAAGCATAAAGTGTAAAGCCT |
| | | | GGGGTGCCTAATGAGTGAGCTAACTCACATTAATT |
| | | | GCGTTGCGCTCACTGCCCGCTTTCCAGTCGGGAAA |
| | | | CCTGTCGTGCCAGCTGCATTAATGAATCGGCCAAC |
| | | | GCGCGGGGAGAGGCGGTTTGCGTATTGGGCGCTCT |
| | | | TCCGCTTCCTCGCTCACTGACTCGCTGCGCTCGGTC |
| | | | GTTCGGCTGCGGCGAGCGGTATCAGCTCACTCAAA |
| | | | GGCGGTAATACGGTTATCCACAGAATCAGGGGATA |
| | | | ACGCAGGAAAGAACATGTGAGCAAAAGGCCAGCA |
| | | | AAAGGCCAGGAACCGTAAAAAGGCCGCGTTGCTG |
| | | | GCGTTTTTCCATAGGCTCCGCCCCCCTGACGAGCAT |
| | | | CACAAAAATCGACGCTCAAGTCAGAGGTGGCGAA |
| | | | ACCCGACAGGACTATAAAGATACCAGGCGTTTCCC |
| | | | CCTGGAAGCTCCCTCGTGCGCTCTCCTGTTCCGACC |
| | | | CTGCCGCTTACCGGATACCTGTCCGCCTTTCTCCCT |
| | | | TCGGGAAGCGTGGCGCTTTCTCATAGCTCACGCTGT |
| | | | AGGTATCTCAGTTCGGTGTAGGTCGTTCGCTCCAAG |
| | | | CTGGGCTGTGTGCACGAACCCCCCGTTCAGCCCGA |
| | | | CCGCTGCGCCTTATCCGGTAACTATCGTCTTGAGTC |
| | | | CAACCCGGTAAGACACGACTTATCGCCACTGGCAG |
| | | | CAGCCACTGGTAACAGGATTAGCAGAGCGAGGTAT |
| | | | GTAGGCGGTGCTACAGAGTTCTTGAAGTGGTGGCC |
| | | | TAACTACGGCTACACTAGAAGAACAGTATTTGGTA |
| | | | TCTGCGCTCTGCTGAAGCCAGTTACCTTCGGAAAA |
| | | | AGAGTTGGTAGCTCTTGATCCGGCAAACAAACCAC |
| | | | CGCTGGTAGCGGTGGTTTTTTTGTTTGCAAGCAGCA |
| | | | GATTACGCGCAGAAAAAAAGGATCTCAAGAAGAT |
| | | | CCTTTGATCTTTTCTACGGGGTCTGACGCTCAGTGG |
| | | | AACGAAAACTCACGTTAAGGGATTTTGGTCATGAG |
| | | | ATTATCAAAAAGGATCTTCACCTAGATCCTTTTAAA |
| | | | TTAAAAATGAAGTTTTAAATCAATCTAAAGTATAT |
| | | | ATGAGTAAACTTGGTCTGACAGTTAGAAAAACTCA |
| | | | TCGAGCATCAAATGAAACTGCAATTTATTCATATC |
| | | | AGGATTATCAATACCATATTTTTGAAAAAGCCGTTT |
| | | | CTGTAATGAAGGAGAAAACTCACCGAGGCAGTTCC |
| | | | ATAGGATGGCAAGATCCTGGTATCGGTCTGCGATT |
| | | | CCGACTCGTCCAACATCAATACAACCTATTAATTTC |
| | | | CCCTCGTCAAAAATAAGGTTATCAAGTGAGAAATC |
| | | | ACCATGAGTGACGACTGAATCCGGTGAGAATGGCA |
| | | | AAAGTTTATGCATTTCTTTCCAGACTTGTTCAACAG |
| | | | GCCAGCCATTACGCTCGTCATCAAAATCACTCGCA |
| | | | TCAACCAAACCGTTATTCATTCGTGATTGCGCCTGA |
| | | | GCGAGACGAAATACGCGATCGCTGTTAAAAGGACA |
| | | | ATTACAAACAGGAATCGAATGCAACCGGCGCAGG |
| | | | AACACTGCCAGCGCATCAACAATATTTTCACCTGA |
| | | | ATCAGGATATTCTTCTAATACCTGGAATGCTGTTTT |
| | | | CCCAGGGATCGCAGTGGTGAGTAACCATGCATCAT |
| | | | CAGGAGTACGGATAAAATGCTTGATGGTCGGAAGA |
| | | | GGCATAAATTCCGTCAGCCAGTTTAGTCTGACCATC |
| | | | TCATCTGTAACATCATTGGCAACGCTACCTTTGCCA |
| | | | TGTTTCAGAAACAACTCTGGCGCATCGGGCTTCCC |
| | | | ATACAATCGATAGATTGTCGCACCTGATTGCCCGA |
| | | | CATTATCGCGAGCCCATTTATACCCATATAAATCAG |
| | | | CATCCATGTTGGAATTTAATCGCGGCCTAGAGCAA |
| | | | GACGTTTCCCGTTGAATATGGCTCATACTCTTCCTT |
| | | | TTTCAATATTATTGAAGCATTTATCAGGGTTATTGT |
| | | | CTCATGAGCGGATACATATTTGAATGTATTTAGAA |
| | | | AAATAAACAAATAGGGGTTCCGCGCACATTTCCCC |
| | | | GAAAAGTGCCACCTGACGTCTAAGAAACCATTATT |
| | | | ATCATGACATTAACCTATAAAAATAGGCGTATCAC |
| | | | GAGGCCCTTTCGTC |
| 67 | pYZZ456 | Rep2Cap2-p5p5 | TCGCGCGTTTCGGTGATGACGGTGAAAACCTCTGA |
| | | | CACATGCAGCTCCCGGAGACGGTCACAGCTTGTCT |
| | | | GTAAGCGGATGCCGGGAGCAGACAAGCCCGTCAG |
| | | | GGCGCGTCAGCGGGTGTTGGCGGGTGTCGGGGCTG |
| | | | GCTTAACTATGCGGCATCAGAGCAGATTGTACTGA |
| | | | GAGTGCACCATATGCGGTGTGAAATACCGCACAGA |
| | | | TGCGTAAGGAGAAAATACCGCATCAGGCGCCATTC |
| | | | GCCATTCAGGCTGCGCAACTGTTGGGAAGGGCGAT |
| | | | CGGTGCGGGCCTCTTCGCTATTACGCCAGCTGGCG |
| | | | AAAGGGGGATGTGCTGCAAGGCGATTAAGTTGGGT |
| | | | AACGCCAGGGTTTTCCCAGTCACGACGTTGTAAAA |
| | | | CGACGGCCAGTGAATTGACGCGTATTGGGATCGCG |
| | | | CAGCCGCCATGCCGGGGTTTTACGAGATTGTGATT |
| | | | AAGGTCCCCAGCGACCTTGACGAGCATCTGCCCGG |
| | | | CATTTCTGACAGCTTTGTGAACTGGGTGGCCGAGA |
| | | | AGGAATGGGAGTTGCCGCCAGATTCTGACATGGAT |
| | | | CTGAATCTGATTGAGCAGGCACCCCTGACCGTGGC |
| | | | CGAGAAGCTGCAGCGCGACTTTCTGACGGAATGGC |
| | | | GCCGTGTGAGTAAGGCCCCGGAGGCTCTTTTCTTTG |
| | | | TGCAATTTGAGAAGGGAGAGAGCTACTTCCACATG |
| | | | CACGTGCTCGTGGAAACCACCGGGGTGAAATCCAT |
| | | | GGTTTTGGGACGTTTCCTGAGTCAGATTCGCGAAA |
| | | | AACTGATTCAGAGAATTTACCGCGGGATCGAGCCG |
| | | | ACTTTGCCAAACTGGTTCGCGGTCACAAAGACCAG |
| | | | AAATGGCGCCGGAGGCGGGAACAAGGTGGTGGAT |
| | | | GAGTGCTACATCCCCAATTACTTGCTCCCCAAAACC |
| | | | CAGCCTGAGCTCCAGTGGGCGTGGACTAATATGGA |
| | | | ACAGTATTTAAGCGCCTGTTTGAATCTCACGGAGC |
| | | | GTAAACGGTTGGTGGCGCAGCATCTGACGCACGTG |
| | | | TCGCAGACGCAGGAGCAGAACAAAGAGAATCAGA |
| | | | ATCCCAATTCTGATGCGCCGGTGATCAGATCAAAA |
| | | | ACTTCAGCCAGGTACATGGAGCTGGTCGGGTGGCT |
| | | | CGTGGACAAGGGGATTACCTCGGAGAAGCAGTGG |
| | | | ATCCAGGAGGACCAGGCCTCATACATCTCCTTCAA |
| | | | TGCGGCCTCCAACTCGCGGTCCCAAATCAAGGCTG |
| | | | CCTTGGACAATGCGGGAAAGATTATGAGCCTGACT |
| | | | AAAACCGCCCCCGACTACCTGGTGGGCCAGCAGCC |
| | | | CGTGGAGGACATTTCCAGCAATCGGATTTATAAAA |
| | | | TTTTGGAACTAAACGGGTACGATCCCCAATATGCG |
| | | | GCTTCCGTCTTTCTGGGATGGGCCACGAAAAAGTT |
| | | | CGGCAAGAGGAACACCATCTGGCTGTTTGGGCCTG |
| | | | CAACTACCGGGAAGACCAACATCGCGGAGGCCATA |
| | | | GCCCACACTGTGCCCTTCTACGGGTGCGTAAACTG |
| | | | GACCAATGAGAACTTTCCCTTCAACGACTGTGTCG |
| | | | ACAAGATGGTGATCTGGTGGGAGGAGGGGAAGAT |
| | | | GACCGCCAAGGTCGTGGAGTCGGCCAAAGCCATTC |
| | | | TCGGAGGAAGCAAGGTGCGCGTGGACCAGAAATG |
| | | | CAAGTCCTCGGCCCAGATAGACCCGACTCCCGTGA |
| | | | TCGTCACCTCCAACACCAACATGTGCGCCGTGATT |
| | | | GACGGGAACTCAACGACCTTCGAACACCAGCAGCC |
| | | | GTTGCAAGACCGGATGTTCAAATTTGAACTCACCC |
| | | | GCCGTCTGGATCATGACTTTGGGAAGGTCACCAAG |
| | | | CAGGAAGTCAAAGACTTTTTCCGGTGGGCAAAGGA |
| | | | TCACGTGGTTGAGGTGGAGCATGAATTCTACGTCA |
| | | | AAAAGGGTGGAGCCAAGAAAAGACCCGCCCCCAG |
| | | | TGACGCAGATATAAGTGAGCCCAAACGGGTGCGCG |
| | | | AGTCAGTTGCGCAGCCATCGACGTCAGACGCGGAA |
| | | | GCTTCGATCAACTACGCAGACAGGTACCAAAACAA |
| | | | ATGTTCTCGTCACGTGGGCATGAATCTGATGCTGTT |
| | | | TCCCTGCAGACAATGCGAGAGAATGAATCAGAATT |
| | | | CAAATATCTGCTTCACTCACGGACAGAAAGACTGT |
| | | | TTAGAGTGCTTTCCCGTGTCAGAATCTCAACCCGTT |
| | | | TCTGTCGTCAAAAAGGCGTATCAGAAACTGTGCTA |
| | | | CATTCATCATATCATGGGAAAGGTGCCAGACGCTT |
| | | | GCACTGCCTGCGATCTGGTCAATGTGGATTTGGAT |
| | | | GACTGCATCTTTGAACAATAAATGATTTAAATCAG |
| | | | GTatggctgccgatggttatcttccagattggctcgaggacactctctctgaaggaat |
| | | | aagacagtggtggaagctcaaacctggcccaccaccaccaaagcccgcagagcg |
| | | | gcataaggacgacagcaggggtcttgtgcttcctgggtacaagtacctcggaccctt |
| | | | caacggactcgacaagggagagccggtcaacgaggcagacgccgcggccctcg |
| | | | agcacgacaaagcctacgaccggcagctcgacagcggagacaacccgtacctca |
| | | | agtacaaccacgccgacgcggagtttcaggagcgccttaaagaagatacgtcttttg |
| | | | ggggcaacctcggacgagcagtcttccaggcgaaaaagagggttcttgaacctctg |
| | | | ggcctggttgaggaacctgttaagacggctccgggaaaaaagaggccggtagagc |
| | | | actctcctgtggagccagactcctcctcgggaaccggaaagggggccagcagcc |
| | | | tgcaagaaaaagattgaattttggtcagactggagacgcagactcagtacctgaccc |
| | | | ccagcctctcggacagccaccagcagccccctctggtctgggaactaatacgatgg |
| | | | ctacaggcagtggcgcaccaatggcagacaataacgagggcgccgacggagtgg |
| | | | gtaattcctcgggaaattggcattgcgattccacatggatgggcgacagagtcatcac |
| | | | caccagcacccgaacctgggccctgcccacctacaacaaccacctctacaaacaaa |
| | | | tttccagccaatcaggagcctcgaacgacaatcactactttggctacagcaccccttg |
| | | | ggggtattttgacttcaacagattccactgccacttttcaccacgtgactggcaaagac |
| | | | tcatcaacaacaactggggattccgacccaagagactcaacttcaagctctttaacatt |
| | | | caagtcaaagaggtcacgcagaatgacggtacgacgacgattgccaataaccttac |
| | | | cagcacggttcaggtgtttactgactcggagtaccagctcccgtacgtcctcggctcg |
| | | | gcgcatcaaggatgcctcccgccgttcccagcagacgtcttcatggtgccacagtat |
| | | | ggatacctcaccctgaacaacgggagtcaggcagtaggacgctcttcattttactgcc |
| | | | tggagtactttccttctcagatgctgcgtaccggaaacaactttaccttcagctacacttt |
| | | | tgaggacgttcctttccacagcagctacgctcacagccagagtctggaccgtctcatg |
| | | | aatcctctcatcgaccagtacctgtattacttgagcagaacaaacactccaagtggaa |
| | | | ccaccacgcagtcaaggcttcagttttctcaggccggagcgagtgacattcgggacc |
| | | | agtctaggaactggcttcctggaccctgttaccgccagcagcgagtatcaaagacat |
| | | | ctgcggataacaacaacagtgaatactcgtggactggagctaccaagtaccacctca |
| | | | atggcagagactctctggtgaatccgggcccggccatggcaagccacaaggacga |
| | | | tgaagaaaagttttttcctcagagcggggttctcatctttgggaagcaaggctcagag |
| | | | aaaacaaatgtggacattgaaaaggtcatgattacagacgaagaggaaatcaggac |
| | | | aaccaatcccgtggctacggagcagtatggttctgtatctaccaacctccagagagg |
| | | | caacagacaagcagctaccgcagatgtcaacacacaaggcgttcttccaggcatgg |
| | | | tctggcaggacagagatgtgtaccttcaggggcccatctgggcaaagattccacaca |
| | | | cggacggacattttcacccctctcccctcatgggtggattcggacttaaacaccctcct |
| | | | ccacagattctcatcaagaacaccccggtacctgcgaatccttcgaccaccttcagtg |
| | | | cggcaaagtttgcttccttcatcacacagtactccacgggacaggtcagcgtggaga |
| | | | tcgagtgggagctgcagaaggaaaacagcaaacgctggaatcccgaaattcagta |
| | | | cacttccaactacaacaagtctgttaatgtggactttactgtggacactaatggcgtgta |
| | | | ttcagagcctcgccccattggcaccagatacctgactcgtaatctgtaaTTGCTT |
| | | | GTTAATCAATAAACCGTTTAATTCGTTTCAGTTGAA |
| | | | CTTTGGTCTCTGCCCTAGGGGAGGTCCTGTATTAGA |
| | | | GGTCACGTGAGTGTTTTGCGACATTTTGCGACACCA |
| | | | TGTGGTCACGCTGGGTATTTAAGCCCGAGTGAGCA |
| | | | CGCAGGGTCTCCATTTTGAAGCGGGAGGTTTGAAcg |
| | | | cgcagccgccaagccgtttcagttgaactttggtctctgcgtatttctttcttatctagtt |
| | | | tccatggaggtcctgtattagaggtcacgtgagtgttttgcgacattttgcgacaccatg |
| | | | tggtcacgctgggtatttaagcccgagtgagcacgcagggtctccattttgaagcgg |
| | | | gaggtttgaaATCCCAATGGCGCGCCGAGCTTGGCTCGA |
| | | | GCATGGTCATAGCTGTTTCCTGTGTGAAATTGTTAT |
| | | | CCGCTCACAATTCCACACAACATACGAGCCGGAAG |
| | | | CATAAAGTGTAAAGCCTGGGGTGCCTAATGAGTGA |
| | | | GCTAACTCACATTAATTGCGTTGCGCTCACTGCCCG |
| | | | CTTTCCAGTCGGGAAACCTGTCGTGCCAGCTGCATT |
| | | | AATGAATCGGCCAACGCGCGGGGAGAGGCGGTTTG |
| | | | CGTATTGGGCGCTCTTCCGCTTCCTCGCTCACTGAC |
| | | | TCGCTGCGCTCGGTCGTTCGGCTGCGGCGAGCGGT |
| | | | ATCAGCTCACTCAAAGGCGGTAATACGGTTATCCA |
| | | | CAGAATCAGGGGATAACGCAGGAAAGAACATGTG |
| | | | AGCAAAAGGCCAGCAAAAGGCCAGGAACCGTAAA |
| | | | AAGGCCGCGTTGCTGGCGTTTTTCCATAGGCTCCGC |
| | | | CCCCCTGACGAGCATCACAAAAATCGACGCTCAAG |
| | | | TCAGAGGTGGCGAAACCCGACAGGACTATAAAGAT |
| | | | ACCAGGCGTTTCCCCCTGGAAGCTCCCTCGTGCGCT |
| | | | CTCCTGTTCCGACCCTGCCGCTTACCGGATACCTGT |
| | | | CCGCCTTTCTCCCTTCGGGAAGCGTGGCGCTTTCTC |
| | | | ATAGCTCACGCTGTAGGTATCTCAGTTCGGTGTAG |
| | | | GTCGTTCGCTCCAAGCTGGGCTGTGTGCACGAACC |
| | | | CCCCGTTCAGCCCGACCGCTGCGCCTTATCCGGTAA |
| | | | CTATCGTCTTGAGTCCAACCCGGTAAGACACGACT |
| | | | TATCGCCACTGGCAGCAGCCACTGGTAACAGGATT |
| | | | AGCAGAGCGAGGTATGTAGGCGGTGCTACAGAGTT |
| | | | CTTGAAGTGGTGGCCTAACTACGGCTACACTAGAA |
| | | | GAACAGTATTTGGTATCTGCGCTCTGCTGAAGCCA |
| | | | GTTACCTTCGGAAAAAGAGTTGGTAGCTCTTGATC |
| | | | CGGCAAACAAACCACCGCTGGTAGCGGTGGTTTTT |
| | | | TTGTTTGCAAGCAGCAGATTACGCGCAGAAAAAAA |
| | | | GGATCTCAAGAAGATCCTTTGATCTTTTCTACGGGG |
| | | | TCTGACGCTCAGTGGAACGAAAACTCACGTTAAGG |
| | | | GATTTTGGTCATGAGATTATCAAAAAGGATCTTCA |
| | | | CCTAGATCCTTTTAAATTAAAAATGAAGTTTTAAAT |
| | | | CAATCTAAAGTATATATGAGTAAACTTGGTCTGAC |
| | | | AGTTAGAAAAACTCATCGAGCATCAAATGAAACTG |
| | | | CAATTTATTCATATCAGGATTATCAATACCATATTT |
| | | | TTGAAAAAGCCGTTTCTGTAATGAAGGAGAAAACT |
| | | | CACCGAGGCAGTTCCATAGGATGGCAAGATCCTGG |
| | | | TATCGGTCTGCGATTCCGACTCGTCCAACATCAATA |
| | | | CAACCTATTAATTTCCCCTCGTCAAAAATAAGGTTA |
| | | | TCAAGTGAGAAATCACCATGAGTGACGACTGAATC |
| | | | CGGTGAGAATGGCAAAAGTTTATGCATTTCTTTCCA |
| | | | GACTTGTTCAACAGGCCAGCCATTACGCTCGTCATC |
| | | | AAAATCACTCGCATCAACCAAACCGTTATTCATTC |
| | | | GTGATTGCGCCTGAGCGAGACGAAATACGCGATCG |
| | | | CTGTTAAAAGGACAATTACAAACAGGAATCGAATG |
| | | | CAACCGGCGCAGGAACACTGCCAGCGCATCAACAA |
| | | | TATTTTCACCTGAATCAGGATATTCTTCTAATACCT |
| | | | GGAATGCTGTTTTCCCAGGGATCGCAGTGGTGAGT |
| | | | AACCATGCATCATCAGGAGTACGGATAAAATGCTT |
| | | | GATGGTCGGAAGAGGCATAAATTCCGTCAGCCAGT |
| | | | TTAGTCTGACCATCTCATCTGTAACATCATTGGCAA |
| | | | CGCTACCTTTGCCATGTTTCAGAAACAACTCTGGCG |
| | | | CATCGGGCTTCCCATACAATCGATAGATTGTCGCA |
| | | | CCTGATTGCCCGACATTATCGCGAGCCCATTTATAC |
| | | | CCATATAAATCAGCATCCATGTTGGAATTTAATCGC |
| | | | GGCCTAGAGCAAGACGTTTCCCGTTGAATATGGCT |
| | | | CATACTCTTCCTTTTTCAATATTATTGAAGCATTTAT |
| | | | CAGGGTTATTGTCTCATGAGCGGATACATATTTGA |
| | | | ATGTATTTAGAAAAATAAACAAATAGGGGTTCCGC |
| | | | GCACATTTCCCCGAAAAGTGCCACCTGACGTCTAA |
| | | | GAAACCATTATTATCATGACATTAACCTATAAAAA |
| | | | TAGGCGTATCACGAGGCCCTTTCGTC |
| 68 | pYZZ457 | Rep2Cap2-p5p5 | TCGCGCGTTTCGGTGATGACGGTGAAAACCTCTGA |
| | | | CACATGCAGCTCCCGGAGACGGTCACAGCTTGTCT |
| | | | GTAAGCGGATGCCGGGAGCAGACAAGCCCGTCAG |
| | | | GGCGCGTCAGCGGGTGTTGGCGGGTGTCGGGGCTG |
| | | | GCTTAACTATGCGGCATCAGAGCAGATTGTACTGA |
| | | | GAGTGCACCATATGCGGTGTGAAATACCGCACAGA |
| | | | TGCGTAAGGAGAAAATACCGCATCAGGCGCCATTC |
| | | | GCCATTCAGGCTGCGCAACTGTTGGGAAGGGCGAT |
| | | | CGGTGCGGGCCTCTTCGCTATTACGCCAGCTGGCG |
| | | | AAAGGGGGATGTGCTGCAAGGCGATTAAGTTGGGT |
| | | | AACGCCAGGGTTTTCCCAGTCACGACGTTGTAAAA |
| | | | CGACGGCCAGTGAATTGACGCGTATTGGGATCGCG |
| | | | CAGCCGCCATGCCGGGGTTTTACGAGATTGTGATT |
| | | | AAGGTCCCCAGCGACCTTGACGAGCATCTGCCCGG |
| | | | CATTTCTGACAGCTTTGTGAACTGGGTGGCCGAGA |
| | | | AGGAATGGGAGTTGCCGCCAGATTCTGACATGGAT |
| | | | CTGAATCTGATTGAGCAGGCACCCCTGACCGTGGC |
| | | | CGAGAAGCTGCAGCGCGACTTTCTGACGGAATGGC |
| | | | GCCGTGTGAGTAAGGCCCCGGAGGCTCTTTTCTTTG |
| | | | TGCAATTTGAGAAGGGAGAGAGCTACTTCCACATG |
| | | | CACGTGCTCGTGGAAACCACCGGGGTGAAATCCAT |
| | | | GGTTTTGGGACGTTTCCTGAGTCAGATTCGCGAAA |
| | | | AACTGATTCAGAGAATTTACCGCGGGATCGAGCCG |
| | | | ACTTTGCCAAACTGGTTCGCGGTCACAAAGACCAG |
| | | | AAATGGCGCCGGAGGCGGGAACAAGGTGGTGGAT |
| | | | GAGTGCTACATCCCCAATTACTTGCTCCCCAAAACC |
| | | | CAGCCTGAGCTCCAGTGGGCGTGGACTAATATGGA |
| | | | ACAGTATTTAAGCGCCTGTTTGAATCTCACGGAGC |
| | | | GTAAACGGTTGGTGGCGCAGCATCTGACGCACGTG |
| | | | TCGCAGACGCAGGAGCAGAACAAAGAGAATCAGA |
| | | | ATCCCAATTCTGATGCGCCGGTGATCAGATCAAAA |
| | | | ACTTCAGCCAGGTACATGGAGCTGGTCGGGTGGCT |
| | | | CGTGGACAAGGGGATTACCTCGGAGAAGCAGTGG |
| | | | ATCCAGGAGGACCAGGCCTCATACATCTCCTTCAA |
| | | | TGCGGCCTCCAACTCGCGGTCCCAAATCAAGGCTG |
| | | | CCTTGGACAATGCGGGAAAGATTATGAGCCTGACT |
| | | | AAAACCGCCCCCGACTACCTGGTGGGCCAGCAGCC |
| | | | CGTGGAGGACATTTCCAGCAATCGGATTTATAAAA |
| | | | TTTTGGAACTAAACGGGTACGATCCCCAATATGCG |
| | | | GCTTCCGTCTTTCTGGGATGGGCCACGAAAAAGTT |
| | | | CGGCAAGAGGAACACCATCTGGCTGTTTGGGCCTG |
| | | | CAACTACCGGGAAGACCAACATCGCGGAGGCCATA |
| | | | GCCCACACTGTGCCCTTCTACGGGTGCGTAAACTG |
| | | | GACCAATGAGAACTTTCCCTTCAACGACTGTGTCG |
| | | | ACAAGATGGTGATCTGGTGGGAGGAGGGGAAGAT |
| | | | GACCGCCAAGGTCGTGGAGTCGGCCAAAGCCATTC |
| | | | TCGGAGGAAGCAAGGTGCGCGTGGACCAGAAATG |
| | | | CAAGTCCTCGGCCCAGATAGACCCGACTCCCGTGA |
| | | | TCGTCACCTCCAACACCAACATGTGCGCCGTGATT |
| | | | GACGGGAACTCAACGACCTTCGAACACCAGCAGCC |
| | | | GTTGCAAGACCGGATGTTCAAATTTGAACTCACCC |
| | | | GCCGTCTGGATCATGACTTTGGGAAGGTCACCAAG |
| | | | CAGGAAGTCAAAGACTTTTTCCGGTGGGCAAAGGA |
| | | | TCACGTGGTTGAGGTGGAGCATGAATTCTACGTCA |
| | | | AAAAGGGTGGAGCCAAGAAAAGACCCGCCCCCAG |
| | | | TGACGCAGATATAAGTGAGCCCAAACGGGTGCGCG |
| | | | AGTCAGTTGCGCAGCCATCGACGTCAGACGCGGAA |
| | | | GCTTCGATCAACTACGCAGACAGGTACCAAAACAA |
| | | | ATGTTCTCGTCACGTGGGCATGAATCTGATGCTGTT |
| | | | TCCCTGCAGACAATGCGAGAGAATGAATCAGAATT |
| | | | CAAATATCTGCTTCACTCACGGACAGAAAGACTGT |
| | | | TTAGAGTGCTTTCCCGTGTCAGAATCTCAACCCGTT |
| | | | TCTGTCGTCAAAAAGGCGTATCAGAAACTGTGCTA |
| | | | CATTCATCATATCATGGGAAAGGTGCCAGACGCTT |
| | | | GCACTGCCTGCGATCTGGTCAATGTGGATTTGGAT |
| | | | GACTGCATCTTTGAACAATAAATGATTTAAATCAG |
| | | | GTatggctgccgatggttatcttccagattggctcgaggacactctctctgaaggaat |
| | | | aagacagtggtggaagctcaaacctggcccaccaccaccaaagcccgcagagcg |
| | | | gcataaggacgacagcaggggtcttgtgcttcctgggtacaagtacctcggaccctt |
| | | | caacggactcgacaagggagagccggtcaacgaggcagacgccgcggccctcg |
| | | | agcacgacaaagcctacgaccggcagctcgacagcggagacaacccgtacctca |
| | | | agtacaaccacgccgacgcggagtttcaggagcgccttaaagaagatacgtcttttg |
| | | | ggggcaacctcggacgagcagtcttccaggcgaaaaagagggttcttgaacctctg |
| | | | ggcctggttgaggaacctgttaagacggctccgggaaaaaagaggccggtagagc |
| | | | actctcctgtggagccagactcctcctcgggaaccggaaagggggccagcagcc |
| | | | tgcaagaaaaagattgaattttggtcagactggagacgcagactcagtacctgaccc |
| | | | ccagcctctcggacagccaccagcagccccctctggtctgggaactaatacgatgg |
| | | | ctacaggcagtggcgcaccaatggcagacaataacgagggcgccgacggagtgg |
| | | | gtaattcctcgggaaattggcattgcgattccacatggatgggcgacagagtcatcac |
| | | | caccagcacccgaacctgggccctgcccacctacaacaaccacctctacaaacaaa |
| | | | tttccagccaatcaggagcctcgaacgacaatcactactttggctacagcaccccttg |
| | | | ggggtattttgacttcaacagattccactgccacttttcaccacgtgactggcaaagac |
| | | | tcatcaacaacaactggggattccgacccaagagactcaacttcaagctctttaacatt |
| | | | caagtcaaagaggtcacgcagaatgacggtacgacgacgattgccaataaccttac |
| | | | cagcacggttcaggtgtttactgactcggagtaccagctcccgtacgtcctcggctcg |
| | | | gcgcatcaaggatgcctcccgccgttcccagcagacgtcttcatggtgccacagtat |
| | | | ggatacctcaccctgaacaacgggagtcaggcagtaggacgctcttcattttactgcc |
| | | | tggagtactttccttctcagatgctgcgtaccggaaacaactttaccttcagctacacttt |
| | | | tgaggacgttcctttccacagcagctacgctcacagccagagtctggaccgtctcatg |
| | | | aatcctctcatcgaccagtacctgtattacttgagcagaacaaacactccaagtggaa |
| | | | ccaccacgcagtcaaggcttcagttttctcaggccggagcgagtgacattcgggacc |
| | | | agtctaggaactggcttcctggaccctgttaccgccagcagcgagtatcaaagacat |
| | | | ctgcggataacaacaacagtgaatactcgtggactggagctaccaagtaccacctca |
| | | | atggcagagactctctggtgaatccgggcccggccatggcaagccacaaggacga |
| | | | tgaagaaaagttttttcctcagagcggggttctcatctttgggaagcaaggctcagag |
| | | | aaaacaaatgtggacattgaaaaggtcatgattacagacgaagaggaaatcaggac |
| | | | aaccaatcccgtggctacggagcagtatggttctgtatctaccaacctccagagagg |
| | | | caacagacaagcagctaccgcagatgtcaacacacaaggcgttcttccaggcatgg |
| | | | tctggcaggacagagatgtgtaccttcaggggcccatctgggcaaagattccacaca |
| | | | cggacggacattttcacccctctcccctcatgggtggattcggacttaaacaccctcct |
| | | | ccacagattctcatcaagaacaccccggtacctgcgaatccttcgaccaccttcagtg |
| | | | cggcaaagtttgcttccttcatcacacagtactccacgggacaggtcagcgtggaga |
| | | | tcgagtgggagctgcagaaggaaaacagcaaacgctggaatcccgaaattcagta |
| | | | cacttccaactacaacaagtctgttaatgtggactttactgtggacactaatggcgtgta |
| | | | ttcagagcctcgccccattggcaccagatacctgactcgtaatctgtaaTTGCTT |
| | | | GTTAATCAATAAACCGTTTAATTCGTTTCAGTTGAA |
| | | | CTTTGGTCTCTGCCCTAGGGGAGGTCCTGTATTAGA |
| | | | GGTCACGTGAGTGTTTTGCGACATTTTGCGACACCA |
| | | | TGTGGTCACGCTGGGTATTTAAGCCCGAGTGAGCA |
| | | | CGCAGGGTCTCCATTTTGAAGCGGGAGGTTTGAAG |
| | | | TCTTACCCCCAATAAGGGGATTTGCTCTATTTAATT |
| | | | AGGAATAAGGTCGATTACTGATAGAACAAATCCAG |
| | | | GCTACTGTGTTTAGTAATCAGATTTGTTgaggtcctgtatta |
| | | | gaggtcacgtgagtgttttgcgacattttgcgacaccatgtggtcacgctgggtattta |
| | | | agcccgagtgagcacgcagggtctccattttgaagcgggaggtttgaaATCCC |
| | | | AATGGCGCGCCGAGCTTGGCTCGAGCATGGTCATA |
| | | | GCTGTTTCCTGTGTGAAATTGTTATCCGCTCACAAT |
| | | | TCCACACAACATACGAGCCGGAAGCATAAAGTGTA |
| | | | AAGCCTGGGGTGCCTAATGAGTGAGCTAACTCACA |
| | | | TTAATTGCGTTGCGCTCACTGCCCGCTTTCCAGTCG |
| | | | GGAAACCTGTCGTGCCAGCTGCATTAATGAATCGG |
| | | | CCAACGCGCGGGGAGAGGCGGTTTGCGTATTGGGC |
| | | | GCTCTTCCGCTTCCTCGCTCACTGACTCGCTGCGCT |
| | | | CGGTCGTTCGGCTGCGGCGAGCGGTATCAGCTCAC |
| | | | TCAAAGGCGGTAATACGGTTATCCACAGAATCAGG |
| | | | GGATAACGCAGGAAAGAACATGTGAGCAAAAGGC |
| | | | CAGCAAAAGGCCAGGAACCGTAAAAAGGCCGCGT |
| | | | TGCTGGCGTTTTTCCATAGGCTCCGCCCCCCTGACG |
| | | | AGCATCACAAAAATCGACGCTCAAGTCAGAGGTGG |
| | | | CGAAACCCGACAGGACTATAAAGATACCAGGCGTT |
| | | | TCCCCCTGGAAGCTCCCTCGTGCGCTCTCCTGTTCC |
| | | | GACCCTGCCGCTTACCGGATACCTGTCCGCCTTTCT |
| | | | CCCTTCGGGAAGCGTGGCGCTTTCTCATAGCTCACG |
| | | | CTGTAGGTATCTCAGTTCGGTGTAGGTCGTTCGCTC |
| | | | CAAGCTGGGCTGTGTGCACGAACCCCCCGTTCAGC |
| | | | CCGACCGCTGCGCCTTATCCGGTAACTATCGTCTTG |
| | | | AGTCCAACCCGGTAAGACACGACTTATCGCCACTG |
| | | | GCAGCAGCCACTGGTAACAGGATTAGCAGAGCGA |
| | | | GGTATGTAGGCGGTGCTACAGAGTTCTTGAAGTGG |
| | | | TGGCCTAACTACGGCTACACTAGAAGAACAGTATT |
| | | | TGGTATCTGCGCTCTGCTGAAGCCAGTTACCTTCGG |
| | | | AAAAAGAGTTGGTAGCTCTTGATCCGGCAAACAAA |
| | | | CCACCGCTGGTAGCGGTGGTTTTTTTGTTTGCAAGC |
| | | | AGCAGATTACGCGCAGAAAAAAAGGATCTCAAGA |
| | | | AGATCCTTTGATCTTTTCTACGGGGTCTGACGCTCA |
| | | | GTGGAACGAAAACTCACGTTAAGGGATTTTGGTCA |
| | | | TGAGATTATCAAAAAGGATCTTCACCTAGATCCTTT |
| | | | TAAATTAAAAATGAAGTTTTAAATCAATCTAAAGT |
| | | | ATATATGAGTAAACTTGGTCTGACAGTTAGAAAAA |
| | | | CTCATCGAGCATCAAATGAAACTGCAATTTATTCAT |
| | | | ATCAGGATTATCAATACCATATTTTTGAAAAAGCC |
| | | | GTTTCTGTAATGAAGGAGAAAACTCACCGAGGCAG |
| | | | TTCCATAGGATGGCAAGATCCTGGTATCGGTCTGC |
| | | | GATTCCGACTCGTCCAACATCAATACAACCTATTA |
| | | | ATTTCCCCTCGTCAAAAATAAGGTTATCAAGTGAG |
| | | | AAATCACCATGAGTGACGACTGAATCCGGTGAGAA |
| | | | TGGCAAAAGTTTATGCATTTCTTTCCAGACTTGTTC |
| | | | AACAGGCCAGCCATTACGCTCGTCATCAAAATCAC |
| | | | TCGCATCAACCAAACCGTTATTCATTCGTGATTGCG |
| | | | CCTGAGCGAGACGAAATACGCGATCGCTGTTAAAA |
| | | | GGACAATTACAAACAGGAATCGAATGCAACCGGC |
| | | | GCAGGAACACTGCCAGCGCATCAACAATATTTTCA |
| | | | CCTGAATCAGGATATTCTTCTAATACCTGGAATGCT |
| | | | GTTTTCCCAGGGATCGCAGTGGTGAGTAACCATGC |
| | | | ATCATCAGGAGTACGGATAAAATGCTTGATGGTCG |
| | | | GAAGAGGCATAAATTCCGTCAGCCAGTTTAGTCTG |
| | | | ACCATCTCATCTGTAACATCATTGGCAACGCTACCT |
| | | | TTGCCATGTTTCAGAAACAACTCTGGCGCATCGGG |
| | | | CTTCCCATACAATCGATAGATTGTCGCACCTGATTG |
| | | | CCCGACATTATCGCGAGCCCATTTATACCCATATAA |
| | | | ATCAGCATCCATGTTGGAATTTAATCGCGGCCTAG |
| | | | AGCAAGACGTTTCCCGTTGAATATGGCTCATACTCT |
| | | | TCCTTTTTCAATATTATTGAAGCATTTATCAGGGTT |
| | | | ATTGTCTCATGAGCGGATACATATTTGAATGTATTT |
| | | | AGAAAAATAAACAAATAGGGGTTCCGCGCACATTT |
| | | | CCCCGAAAAGTGCCACCTGACGTCTAAGAAACCAT |
| | | | TATTATCATGACATTAACCTATAAAAATAGGCGTA |
| | | | TCACGAGGCCCTTTCGTC |
| 69 | pYZZ458 | Rep2Cap2-p5p5 | TCGCGCGTTTCGGTGATGACGGTGAAAACCTCTGA |
| | | | CACATGCAGCTCCCGGAGACGGTCACAGCTTGTCT |
| | | | GTAAGCGGATGCCGGGAGCAGACAAGCCCGTCAG |
| | | | GGCGCGTCAGCGGGTGTTGGCGGGTGTCGGGGCTG |
| | | | GCTTAACTATGCGGCATCAGAGCAGATTGTACTGA |
| | | | GAGTGCACCATATGCGGTGTGAAATACCGCACAGA |
| | | | TGCGTAAGGAGAAAATACCGCATCAGGCGCCATTC |
| | | | GCCATTCAGGCTGCGCAACTGTTGGGAAGGGCGAT |
| | | | CGGTGCGGGCCTCTTCGCTATTACGCCAGCTGGCG |
| | | | AAAGGGGGATGTGCTGCAAGGCGATTAAGTTGGGT |
| | | | AACGCCAGGGTTTTCCCAGTCACGACGTTGTAAAA |
| | | | CGACGGCCAGTGAATTGACGCGTATTGGGATCGCG |
| | | | CAGCCGCCATGCCGGGGTTTTACGAGATTGTGATT |
| | | | AAGGTCCCCAGCGACCTTGACGAGCATCTGCCCGG |
| | | | CATTTCTGACAGCTTTGTGAACTGGGTGGCCGAGA |
| | | | AGGAATGGGAGTTGCCGCCAGATTCTGACATGGAT |
| | | | CTGAATCTGATTGAGCAGGCACCCCTGACCGTGGC |
| | | | CGAGAAGCTGCAGCGCGACTTTCTGACGGAATGGC |
| | | | GCCGTGTGAGTAAGGCCCCGGAGGCTCTTTTCTTTG |
| | | | TGCAATTTGAGAAGGGAGAGAGCTACTTCCACATG |
| | | | CACGTGCTCGTGGAAACCACCGGGGTGAAATCCAT |
| | | | GGTTTTGGGACGTTTCCTGAGTCAGATTCGCGAAA |
| | | | AACTGATTCAGAGAATTTACCGCGGGATCGAGCCG |
| | | | ACTTTGCCAAACTGGTTCGCGGTCACAAAGACCAG |
| | | | AAATGGCGCCGGAGGCGGGAACAAGGTGGTGGAT |
| | | | GAGTGCTACATCCCCAATTACTTGCTCCCCAAAACC |
| | | | CAGCCTGAGCTCCAGTGGGCGTGGACTAATATGGA |
| | | | ACAGTATTTAAGCGCCTGTTTGAATCTCACGGAGC |
| | | | GTAAACGGTTGGTGGCGCAGCATCTGACGCACGTG |
| | | | TCGCAGACGCAGGAGCAGAACAAAGAGAATCAGA |
| | | | ATCCCAATTCTGATGCGCCGGTGATCAGATCAAAA |
| | | | ACTTCAGCCAGGTACATGGAGCTGGTCGGGTGGCT |
| | | | CGTGGACAAGGGGATTACCTCGGAGAAGCAGTGG |
| | | | ATCCAGGAGGACCAGGCCTCATACATCTCCTTCAA |
| | | | TGCGGCCTCCAACTCGCGGTCCCAAATCAAGGCTG |
| | | | CCTTGGACAATGCGGGAAAGATTATGAGCCTGACT |
| | | | AAAACCGCCCCCGACTACCTGGTGGGCCAGCAGCC |
| | | | CGTGGAGGACATTTCCAGCAATCGGATTTATAAAA |
| | | | TTTTGGAACTAAACGGGTACGATCCCCAATATGCG |
| | | | GCTTCCGTCTTTCTGGGATGGGCCACGAAAAAGTT |
| | | | CGGCAAGAGGAACACCATCTGGCTGTTTGGGCCTG |
| | | | CAACTACCGGGAAGACCAACATCGCGGAGGCCATA |
| | | | GCCCACACTGTGCCCTTCTACGGGTGCGTAAACTG |
| | | | GACCAATGAGAACTTTCCCTTCAACGACTGTGTCG |
| | | | ACAAGATGGTGATCTGGTGGGAGGAGGGGAAGAT |
| | | | GACCGCCAAGGTCGTGGAGTCGGCCAAAGCCATTC |
| | | | TCGGAGGAAGCAAGGTGCGCGTGGACCAGAAATG |
| | | | CAAGTCCTCGGCCCAGATAGACCCGACTCCCGTGA |
| | | | TCGTCACCTCCAACACCAACATGTGCGCCGTGATT |
| | | | GACGGGAACTCAACGACCTTCGAACACCAGCAGCC |
| | | | GTTGCAAGACCGGATGTTCAAATTTGAACTCACCC |
| | | | GCCGTCTGGATCATGACTTTGGGAAGGTCACCAAG |
| | | | CAGGAAGTCAAAGACTTTTTCCGGTGGGCAAAGGA |
| | | | TCACGTGGTTGAGGTGGAGCATGAATTCTACGTCA |
| | | | AAAAGGGTGGAGCCAAGAAAAGACCCGCCCCCAG |
| | | | TGACGCAGATATAAGTGAGCCCAAACGGGTGCGCG |
| | | | AGTCAGTTGCGCAGCCATCGACGTCAGACGCGGAA |
| | | | GCTTCGATCAACTACGCAGACAGGTACCAAAACAA |
| | | | ATGTTCTCGTCACGTGGGCATGAATCTGATGCTGTT |
| | | | TCCCTGCAGACAATGCGAGAGAATGAATCAGAATT |
| | | | CAAATATCTGCTTCACTCACGGACAGAAAGACTGT |
| | | | TTAGAGTGCTTTCCCGTGTCAGAATCTCAACCCGTT |
| | | | TCTGTCGTCAAAAAGGCGTATCAGAAACTGTGCTA |
| | | | CATTCATCATATCATGGGAAAGGTGCCAGACGCTT |
| | | | GCACTGCCTGCGATCTGGTCAATGTGGATTTGGAT |
| | | | GACTGCATCTTTGAACAATAAATGATTTAAATCAG |
| | | | GTatggctgccgatggttatcttccagattggctcgaggacactctctctgaaggaat |
| | | | aagacagtggtggaagctcaaacctggcccaccaccaccaaagcccgcagagcg |
| | | | gcataaggacgacagcaggggtcttgtgcttcctgggtacaagtacctcggaccctt |
| | | | caacggactcgacaagggagagccggtcaacgaggcagacgccgcggccctcg |
| | | | agcacgacaaagcctacgaccggcagctcgacagcggagacaacccgtacctca |
| | | | agtacaaccacgccgacgcggagtttcaggagcgccttaaagaagatacgtcttttg |
| | | | ggggcaacctcggacgagcagtcttccaggcgaaaaagagggttcttgaacctctg |
| | | | ggcctggttgaggaacctgttaagacggctccgggaaaaaagaggccggtagagc |
| | | | actctcctgtggagccagactcctcctcgggaaccggaaaggcgggccagcagcc |
| | | | tgcaagaaaaagattgaattttggtcagactggagacgcagactcagtacctgaccc |
| | | | ccagcctctcggacagccaccagcagccccctctggtctgggaactaatacgatgg |
| | | | ctacaggcagtggcgcaccaatggcagacaataacgagggcgccgacggagtgg |
| | | | gtaattcctcgggaaattggcattgcgattccacatggatgggcgacagagtcatcac |
| | | | caccagcacccgaacctgggccctgcccacctacaacaaccacctctacaaacaaa |
| | | | tttccagccaatcaggagcctcgaacgacaatcactactttggctacagcaccccttg |
| | | | ggggtattttgacttcaacagattccactgccacttttcaccacgtgactggcaaagac |
| | | | tcatcaacaacaactggggattccgacccaagagactcaacttcaagctctttaacatt |
| | | | caagtcaaagaggtcacgcagaatgacggtacgacgacgattgccaataaccttac |
| | | | cagcacggttcaggtgtttactgactcggagtaccagctcccgtacgtcctcggctcg |
| | | | gcgcatcaaggatgcctcccgccgttcccagcagacgtcttcatggtgccacagtat |
| | | | ggatacctcaccctgaacaacgggagtcaggcagtaggacgctcttcattttactgcc |
| | | | tggagtactttccttctcagatgctgcgtaccggaaacaactttaccttcagctacacttt |
| | | | tgaggacgttcctttccacagcagctacgctcacagccagagtctggaccgtctcatg |
| | | | aatcctctcatcgaccagtacctgtattacttgagcagaacaaacactccaagtggaa |
| | | | ccaccacgcagtcaaggcttcagttttctcaggccggagcgagtgacattcgggacc |
| | | | agtctaggaactggcttcctggaccctgttaccgccagcagcgagtatcaaagacat |
| | | | ctgcggataacaacaacagtgaatactcgtggactggagctaccaagtaccacctca |
| | | | atggcagagactctctggtgaatccgggcccggccatggcaagccacaaggacga |
| | | | tgaagaaaagttttttcctcagagcggggttctcatctttgggaagcaaggctcagag |
| | | | aaaacaaatgtggacattgaaaaggtcatgattacagacgaagaggaaatcaggac |
| | | | aaccaatcccgtggctacggagcagtatggttctgtatctaccaacctccagagagg |
| | | | caacagacaagcagctaccgcagatgtcaacacacaaggcgttcttccaggcatgg |
| | | | tctggcaggacagagatgtgtaccttcaggggcccatctgggcaaagattccacaca |
| | | | cggacggacattttcacccctctcccctcatgggtggattcggacttaaacaccctcct |
| | | | ccacagattctcatcaagaacaccccggtacctgcgaatccttcgaccaccttcagtg |
| | | | cggcaaagtttgcttccttcatcacacagtactccacgggacaggtcagcgtggaga |
| | | | tcgagtgggagctgcagaaggaaaacagcaaacgctggaatcccgaaattcagta |
| | | | cacttccaactacaacaagtctgttaatgtggactttactgtggacactaatggcgtgta |
| | | | ttcagagcctcgccccattggcaccagatacctgactcgtaatctgtaaTTGCTT |
| | | | GTTAATCAATAAACCGTTTAATTCGTTTCAGTTGAA |
| | | | CTTTGGTCTCTGCCCTAGGGGAGGTCCTGTATTAGA |
| | | | GGTCACGTGAGTGTTTTGCGACATTTTGCGACACCA |
| | | | TGTGGTCACGCTGGGTATTTAAGCCCGAGTGAGCA |
| | | | CGCAGGGTCTCCATTTTGAAGCGGGAGGTTTGAAG |
| | | | TCTTACCCCCAATAAGGGGATTTGCTCTATTTAATT |
| | | | AGGAATAAGGTCGATTACTGATAGAACAAATCCAG |
| | | | GCTACTGTGTTTAGTAATCAGATTTGTTCGTGACCG |
| | | | ATATGCACGGGCAAAACGGCAGGAGGTTGTTAGCG |
| | | | CGACCTCCTGCCACCCGCTTTCACGAAGGTCATGTG |
| | | | TAAAAGGCCGCAGCGTAACTAgaggtcctgtattagaggtcacg |
| | | | tgagtgttttgcgacattttgcgacaccatgtggtcacgctgggtatttaagcccgagt |
| | | | gagcacgcagggtctccattttgaagcgggaggtttgaaATCCCAATGGC |
| | | | GCGCCGAGCTTGGCTCGAGCATGGTCATAGCTGTT |
| | | | TCCTGTGTGAAATTGTTATCCGCTCACAATTCCACA |
| | | | CAACATACGAGCCGGAAGCATAAAGTGTAAAGCCT |
| | | | GGGGTGCCTAATGAGTGAGCTAACTCACATTAATT |
| | | | GCGTTGCGCTCACTGCCCGCTTTCCAGTCGGGAAA |
| | | | CCTGTCGTGCCAGCTGCATTAATGAATCGGCCAAC |
| | | | GCGCGGGGAGAGGCGGTTTGCGTATTGGGCGCTCT |
| | | | TCCGCTTCCTCGCTCACTGACTCGCTGCGCTCGGTC |
| | | | GTTCGGCTGCGGCGAGCGGTATCAGCTCACTCAAA |
| | | | GGCGGTAATACGGTTATCCACAGAATCAGGGGATA |
| | | | ACGCAGGAAAGAACATGTGAGCAAAAGGCCAGCA |
| | | | AAAGGCCAGGAACCGTAAAAAGGCCGCGTTGCTG |
| | | | GCGTTTTTCCATAGGCTCCGCCCCCCTGACGAGCAT |
| | | | CACAAAAATCGACGCTCAAGTCAGAGGTGGCGAA |
| | | | ACCCGACAGGACTATAAAGATACCAGGCGTTTCCC |
| | | | CCTGGAAGCTCCCTCGTGCGCTCTCCTGTTCCGACC |
| | | | CTGCCGCTTACCGGATACCTGTCCGCCTTTCTCCCT |
| | | | TCGGGAAGCGTGGCGCTTTCTCATAGCTCACGCTGT |
| | | | AGGTATCTCAGTTCGGTGTAGGTCGTTCGCTCCAAG |
| | | | CTGGGCTGTGTGCACGAACCCCCCGTTCAGCCCGA |
| | | | CCGCTGCGCCTTATCCGGTAACTATCGTCTTGAGTC |
| | | | CAACCCGGTAAGACACGACTTATCGCCACTGGCAG |
| | | | CAGCCACTGGTAACAGGATTAGCAGAGCGAGGTAT |
| | | | GTAGGCGGTGCTACAGAGTTCTTGAAGTGGTGGCC |
| | | | TAACTACGGCTACACTAGAAGAACAGTATTTGGTA |
| | | | TCTGCGCTCTGCTGAAGCCAGTTACCTTCGGAAAA |
| | | | AGAGTTGGTAGCTCTTGATCCGGCAAACAAACCAC |
| | | | CGCTGGTAGCGGTGGTTTTTTTGTTTGCAAGCAGCA |
| | | | GATTACGCGCAGAAAAAAAGGATCTCAAGAAGAT |
| | | | CCTTTGATCTTTTCTACGGGGTCTGACGCTCAGTGG |
| | | | AACGAAAACTCACGTTAAGGGATTTTGGTCATGAG |
| | | | ATTATCAAAAAGGATCTTCACCTAGATCCTTTTAAA |
| | | | TTAAAAATGAAGTTTTAAATCAATCTAAAGTATAT |
| | | | ATGAGTAAACTTGGTCTGACAGTTAGAAAAACTCA |
| | | | TCGAGCATCAAATGAAACTGCAATTTATTCATATC |
| | | | AGGATTATCAATACCATATTTTTGAAAAAGCCGTTT |
| | | | CTGTAATGAAGGAGAAAACTCACCGAGGCAGTTCC |
| | | | ATAGGATGGCAAGATCCTGGTATCGGTCTGCGATT |
| | | | CCGACTCGTCCAACATCAATACAACCTATTAATTTC |
| | | | CCCTCGTCAAAAATAAGGTTATCAAGTGAGAAATC |
| | | | ACCATGAGTGACGACTGAATCCGGTGAGAATGGCA |
| | | | AAAGTTTATGCATTTCTTTCCAGACTTGTTCAACAG |
| | | | GCCAGCCATTACGCTCGTCATCAAAATCACTCGCA |
| | | | TCAACCAAACCGTTATTCATTCGTGATTGCGCCTGA |
| | | | GCGAGACGAAATACGCGATCGCTGTTAAAAGGACA |
| | | | ATTACAAACAGGAATCGAATGCAACCGGCGCAGG |
| | | | AACACTGCCAGCGCATCAACAATATTTTCACCTGA |
| | | | ATCAGGATATTCTTCTAATACCTGGAATGCTGTTTT |
| | | | CCCAGGGATCGCAGTGGTGAGTAACCATGCATCAT |
| | | | CAGGAGTACGGATAAAATGCTTGATGGTCGGAAGA |
| | | | GGCATAAATTCCGTCAGCCAGTTTAGTCTGACCATC |
| | | | TCATCTGTAACATCATTGGCAACGCTACCTTTGCCA |
| | | | TGTTTCAGAAACAACTCTGGCGCATCGGGCTTCCC |
| | | | ATACAATCGATAGATTGTCGCACCTGATTGCCCGA |
| | | | CATTATCGCGAGCCCATTTATACCCATATAAATCAG |
| | | | CATCCATGTTGGAATTTAATCGCGGCCTAGAGCAA |
| | | | GACGTTTCCCGTTGAATATGGCTCATACTCTTCCTT |
| | | | TTTCAATATTATTGAAGCATTTATCAGGGTTATTGT |
| | | | CTCATGAGCGGATACATATTTGAATGTATTTAGAA |
| | | | AAATAAACAAATAGGGGTTCCGCGCACATTTCCCC |
| | | | GAAAAGTGCCACCTGACGTCTAAGAAACCATTATT |
| | | | ATCATGACATTAACCTATAAAAATAGGCGTATCAC |
| | | | GAGGCCCTTTCGTC |
| 70 | pYZZ459 | Rep2Cap2-p5p5 | TCGCGCGTTTCGGTGATGACGGTGAAAACCTCTGA |
| | | | CACATGCAGCTCCCGGAGACGGTCACAGCTTGTCT |
| | | | GTAAGCGGATGCCGGGAGCAGACAAGCCCGTCAG |
| | | | GGCGCGTCAGCGGGTGTTGGCGGGTGTCGGGGCTG |
| | | | GCTTAACTATGCGGCATCAGAGCAGATTGTACTGA |
| | | | GAGTGCACCATATGCGGTGTGAAATACCGCACAGA |
| | | | TGCGTAAGGAGAAAATACCGCATCAGGCGCCATTC |
| | | | GCCATTCAGGCTGCGCAACTGTTGGGAAGGGCGAT |
| | | | CGGTGCGGGCCTCTTCGCTATTACGCCAGCTGGCG |
| | | | AAAGGGGGATGTGCTGCAAGGCGATTAAGTTGGGT |
| | | | AACGCCAGGGTTTTCCCAGTCACGACGTTGTAAAA |
| | | | CGACGGCCAGTGAATTGACGCGTATTGGGATCGCG |
| | | | CAGCCGCCATGCCGGGGTTTTACGAGATTGTGATT |
| | | | AAGGTCCCCAGCGACCTTGACGAGCATCTGCCCGG |
| | | | CATTTCTGACAGCTTTGTGAACTGGGTGGCCGAGA |
| | | | AGGAATGGGAGTTGCCGCCAGATTCTGACATGGAT |
| | | | CTGAATCTGATTGAGCAGGCACCCCTGACCGTGGC |
| | | | CGAGAAGCTGCAGCGCGACTTTCTGACGGAATGGC |
| | | | GCCGTGTGAGTAAGGCCCCGGAGGCTCTTTTCTTTG |
| | | | TGCAATTTGAGAAGGGAGAGAGCTACTTCCACATG |
| | | | CACGTGCTCGTGGAAACCACCGGGGTGAAATCCAT |
| | | | GGTTTTGGGACGTTTCCTGAGTCAGATTCGCGAAA |
| | | | AACTGATTCAGAGAATTTACCGCGGGATCGAGCCG |
| | | | ACTTTGCCAAACTGGTTCGCGGTCACAAAGACCAG |
| | | | AAATGGCGCCGGAGGCGGGAACAAGGTGGTGGAT |
| | | | GAGTGCTACATCCCCAATTACTTGCTCCCCAAAACC |
| | | | CAGCCTGAGCTCCAGTGGGCGTGGACTAATATGGA |
| | | | ACAGTATTTAAGCGCCTGTTTGAATCTCACGGAGC |
| | | | GTAAACGGTTGGTGGCGCAGCATCTGACGCACGTG |
| | | | TCGCAGACGCAGGAGCAGAACAAAGAGAATCAGA |
| | | | ATCCCAATTCTGATGCGCCGGTGATCAGATCAAAA |
| | | | ACTTCAGCCAGGTACATGGAGCTGGTCGGGTGGCT |
| | | | CGTGGACAAGGGGATTACCTCGGAGAAGCAGTGG |
| | | | ATCCAGGAGGACCAGGCCTCATACATCTCCTTCAA |
| | | | TGCGGCCTCCAACTCGCGGTCCCAAATCAAGGCTG |
| | | | CCTTGGACAATGCGGGAAAGATTATGAGCCTGACT |
| | | | AAAACCGCCCCCGACTACCTGGTGGGCCAGCAGCC |
| | | | CGTGGAGGACATTTCCAGCAATCGGATTTATAAAA |
| | | | TTTTGGAACTAAACGGGTACGATCCCCAATATGCG |
| | | | GCTTCCGTCTTTCTGGGATGGGCCACGAAAAAGTT |
| | | | CGGCAAGAGGAACACCATCTGGCTGTTTGGGCCTG |
| | | | CAACTACCGGGAAGACCAACATCGCGGAGGCCATA |
| | | | GCCCACACTGTGCCCTTCTACGGGTGCGTAAACTG |
| | | | GACCAATGAGAACTTTCCCTTCAACGACTGTGTCG |
| | | | ACAAGATGGTGATCTGGTGGGAGGAGGGGAAGAT |
| | | | GACCGCCAAGGTCGTGGAGTCGGCCAAAGCCATTC |
| | | | TCGGAGGAAGCAAGGTGCGCGTGGACCAGAAATG |
| | | | CAAGTCCTCGGCCCAGATAGACCCGACTCCCGTGA |
| | | | TCGTCACCTCCAACACCAACATGTGCGCCGTGATT |
| | | | GACGGGAACTCAACGACCTTCGAACACCAGCAGCC |
| | | | GTTGCAAGACCGGATGTTCAAATTTGAACTCACCC |
| | | | GCCGTCTGGATCATGACTTTGGGAAGGTCACCAAG |
| | | | CAGGAAGTCAAAGACTTTTTCCGGTGGGCAAAGGA |
| | | | TCACGTGGTTGAGGTGGAGCATGAATTCTACGTCA |
| | | | AAAAGGGTGGAGCCAAGAAAAGACCCGCCCCCAG |
| | | | TGACGCAGATATAAGTGAGCCCAAACGGGTGCGCG |
| | | | AGTCAGTTGCGCAGCCATCGACGTCAGACGCGGAA |
| | | | GCTTCGATCAACTACGCAGACAGGTACCAAAACAA |
| | | | ATGTTCTCGTCACGTGGGCATGAATCTGATGCTGTT |
| | | | TCCCTGCAGACAATGCGAGAGAATGAATCAGAATT |
| | | | CAAATATCTGCTTCACTCACGGACAGAAAGACTGT |
| | | | TTAGAGTGCTTTCCCGTGTCAGAATCTCAACCCGTT |
| | | | TCTGTCGTCAAAAAGGCGTATCAGAAACTGTGCTA |
| | | | CATTCATCATATCATGGGAAAGGTGCCAGACGCTT |
| | | | GCACTGCCTGCGATCTGGTCAATGTGGATTTGGAT |
| | | | GACTGCATCTTTGAACAATAAATGATTTAAATCAG |
| | | | GTatggctgccgatggttatcttccagattggctcgaggacactctctctgaaggaat |
| | | | aagacagtggtggaagctcaaacctggcccaccaccaccaaagcccgcagagcg |
| | | | gcataaggacgacagcaggggtcttgtgcttcctgggtacaagtacctcggaccctt |
| | | | caacggactcgacaagggagagccggtcaacgaggcagacgccgcggccctcg |
| | | | agcacgacaaagcctacgaccggcagctcgacagcggagacaacccgtacctca |
| | | | agtacaaccacgccgacgcggagtttcaggagcgccttaaagaagatacgtcttttg |
| | | | ggggcaacctcggacgagcagtcttccaggcgaaaaagagggttcttgaacctctg |
| | | | ggcctggttgaggaacctgttaagacggctccgggaaaaaagaggccggtagagc |
| | | | actctcctgtggagccagactcctcctcgggaaccggaaaggcgggccagcagcc |
| | | | tgcaagaaaaagattgaattttggtcagactggagacgcagactcagtacctgaccc |
| | | | ccagcctctcggacagccaccagcagccccctctggtctgggaactaatacgatgg |
| | | | ctacaggcagtggcgcaccaatggcagacaataacgagggcgccgacggagtgg |
| | | | gtaattcctcgggaaattggcattgcgattccacatggatgggcgacagagtcatcac |
| | | | caccagcacccgaacctgggccctgcccacctacaacaaccacctctacaaacaaa |
| | | | tttccagccaatcaggagcctcgaacgacaatcactactttggctacagcaccccttg |
| | | | ggggtattttgacttcaacagattccactgccacttttcaccacgtgactggcaaagac |
| | | | tcatcaacaacaactggggattccgacccaagagactcaacttcaagctctttaacatt |
| | | | caagtcaaagaggtcacgcagaatgacggtacgacgacgattgccaataaccttac |
| | | | cagcacggttcaggtgtttactgactcggagtaccagctcccgtacgtcctcggctcg |
| | | | gcgcatcaaggatgcctcccgccgttcccagcagacgtcttcatggtgccacagtat |
| | | | ggatacctcaccctgaacaacgggagtcaggcagtaggacgctcttcattttactgcc |
| | | | tggagtactttccttctcagatgctgcgtaccggaaacaactttaccttcagctacacttt |
| | | | tgaggacgttcctttccacagcagctacgctcacagccagagtctggaccgtctcatg |
| | | | aatcctctcatcgaccagtacctgtattacttgagcagaacaaacactccaagtggaa |
| | | | ccaccacgcagtcaaggcttcagttttctcaggccggagcgagtgacattcgggacc |
| | | | agtctaggaactggcttcctggaccctgttaccgccagcagcgagtatcaaagacat |
| | | | ctgcggataacaacaacagtgaatactcgtggactggagctaccaagtaccacctca |
| | | | atggcagagactctctggtgaatccgggcccggccatggcaagccacaaggacga |
| | | | tgaagaaaagttttttcctcagagcggggttctcatctttgggaagcaaggctcagag |
| | | | aaaacaaatgtggacattgaaaaggtcatgattacagacgaagaggaaatcaggac |
| | | | aaccaatcccgtggctacggagcagtatggttctgtatctaccaacctccagagagg |
| | | | caacagacaagcagctaccgcagatgtcaacacacaaggcgttcttccaggcatgg |
| | | | tctggcaggacagagatgtgtaccttcaggggcccatctgggcaaagattccacaca |
| | | | cggacggacattttcacccctctcccctcatgggtggattcggacttaaacaccctcct |
| | | | ccacagattctcatcaagaacaccccggtacctgcgaatccttcgaccaccttcagtg |
| | | | cggcaaagtttgcttccttcatcacacagtactccacgggacaggtcagcgtggaga |
| | | | tcgagtgggagctgcagaaggaaaacagcaaacgctggaatcccgaaattcagta |
| | | | cacttccaactacaacaagtctgttaatgtggactttactgtggacactaatggcgtgta |
| | | | ttcagagcctcgccccattggcaccagatacctgactcgtaatctgtaaTTGCTT |
| | | | GTTAATCAATAAACCGTTTAATTCGTTTCAGTTGAA |
| | | | CTTTGGTCTCTGCCCTAGGGGAGGTCCTGTATTAGA |
| | | | GGTCACGTGAGTGTTTTGCGACATTTTGCGACACCA |
| | | | TGTGGTCACGCTGGGTATTTAAGCCCGAGTGAGCA |
| | | | CGCAGGGTCTCCATTTTGAAGCGGGAGGTTTGAAG |
| | | | TCTTACCCCCAATAAGGGGATTTGCTCTATTTAATT |
| | | | AGGAATAAGGTCGATTACTGATAGAACAAATCCAG |
| | | | GCTACTGTGTTTAGTAATCAGATTTGTTCGTGACCG |
| | | | ATATGCACGGGCAAAACGGCAGGAGGTTGTTAGCG |
| | | | CGACCTCCTGCCACCCGCTTTCACGAAGGTCATGTG |
| | | | TAAAAGGCCGCAGCGTAACTATTACTAATGAATTC |
| | | | AGGACAGACAGTGGCTACGGCTCAGTTTGGGTTGT |
| | | | GCTGTTGCTGGGCGGCGATGACGCCTGTACGCATT |
| | | | TGGTGATCCGGTTCTGgaggtcctgtattagaggtcacgtgagtgtttt |
| | | | gcgacattttgcgacaccatgtggtcacgctgggtatttaagcccgagtgagcacgc |
| | | | agggtctccattttgaagcgggaggtttgaaATCCCAATGGCGCGCC |
| | | | GAGCTTGGCTCGAGCATGGTCATAGCTGTTTCCTGT |
| | | | GTGAAATTGTTATCCGCTCACAATTCCACACAACAT |
| | | | ACGAGCCGGAAGCATAAAGTGTAAAGCCTGGGGT |
| | | | GCCTAATGAGTGAGCTAACTCACATTAATTGCGTT |
| | | | GCGCTCACTGCCCGCTTTCCAGTCGGGAAACCTGTC |
| | | | GTGCCAGCTGCATTAATGAATCGGCCAACGCGCGG |
| | | | GGAGAGGCGGTTTGCGTATTGGGCGCTCTTCCGCTT |
| | | | CCTCGCTCACTGACTCGCTGCGCTCGGTCGTTCGGC |
| | | | TGCGGCGAGCGGTATCAGCTCACTCAAAGGCGGTA |
| | | | ATACGGTTATCCACAGAATCAGGGGATAACGCAGG |
| | | | AAAGAACATGTGAGCAAAAGGCCAGCAAAAGGCC |
| | | | AGGAACCGTAAAAAGGCCGCGTTGCTGGCGTTTTT |
| | | | CCATAGGCTCCGCCCCCCTGACGAGCATCACAAAA |
| | | | ATCGACGCTCAAGTCAGAGGTGGCGAAACCCGACA |
| | | | GGACTATAAAGATACCAGGCGTTTCCCCCTGGAAG |
| | | | CTCCCTCGTGCGCTCTCCTGTTCCGACCCTGCCGCT |
| | | | TACCGGATACCTGTCCGCCTTTCTCCCTTCGGGAAG |
| | | | CGTGGCGCTTTCTCATAGCTCACGCTGTAGGTATCT |
| | | | CAGTTCGGTGTAGGTCGTTCGCTCCAAGCTGGGCT |
| | | | GTGTGCACGAACCCCCCGTTCAGCCCGACCGCTGC |
| | | | GCCTTATCCGGTAACTATCGTCTTGAGTCCAACCCG |
| | | | GTAAGACACGACTTATCGCCACTGGCAGCAGCCAC |
| | | | TGGTAACAGGATTAGCAGAGCGAGGTATGTAGGCG |
| | | | GTGCTACAGAGTTCTTGAAGTGGTGGCCTAACTAC |
| | | | GGCTACACTAGAAGAACAGTATTTGGTATCTGCGC |
| | | | TCTGCTGAAGCCAGTTACCTTCGGAAAAAGAGTTG |
| | | | GTAGCTCTTGATCCGGCAAACAAACCACCGCTGGT |
| | | | AGCGGTGGTTTTTTTGTTTGCAAGCAGCAGATTACG |
| | | | CGCAGAAAAAAAGGATCTCAAGAAGATCCTTTGAT |
| | | | CTTTTCTACGGGGTCTGACGCTCAGTGGAACGAAA |
| | | | ACTCACGTTAAGGGATTTTGGTCATGAGATTATCA |
| | | | AAAAGGATCTTCACCTAGATCCTTTTAAATTAAAA |
| | | | ATGAAGTTTTAAATCAATCTAAAGTATATATGAGT |
| | | | AAACTTGGTCTGACAGTTAGAAAAACTCATCGAGC |
| | | | ATCAAATGAAACTGCAATTTATTCATATCAGGATT |
| | | | ATCAATACCATATTTTTGAAAAAGCCGTTTCTGTAA |
| | | | TGAAGGAGAAAACTCACCGAGGCAGTTCCATAGGA |
| | | | TGGCAAGATCCTGGTATCGGTCTGCGATTCCGACTC |
| | | | GTCCAACATCAATACAACCTATTAATTTCCCCTCGT |
| | | | CAAAAATAAGGTTATCAAGTGAGAAATCACCATGA |
| | | | GTGACGACTGAATCCGGTGAGAATGGCAAAAGTTT |
| | | | ATGCATTTCTTTCCAGACTTGTTCAACAGGCCAGCC |
| | | | ATTACGCTCGTCATCAAAATCACTCGCATCAACCA |
| | | | AACCGTTATTCATTCGTGATTGCGCCTGAGCGAGA |
| | | | CGAAATACGCGATCGCTGTTAAAAGGACAATTACA |
| | | | AACAGGAATCGAATGCAACCGGCGCAGGAACACT |
| | | | GCCAGCGCATCAACAATATTTTCACCTGAATCAGG |
| | | | ATATTCTTCTAATACCTGGAATGCTGTTTTCCCAGG |
| | | | GATCGCAGTGGTGAGTAACCATGCATCATCAGGAG |
| | | | TACGGATAAAATGCTTGATGGTCGGAAGAGGCATA |
| | | | AATTCCGTCAGCCAGTTTAGTCTGACCATCTCATCT |
| | | | GTAACATCATTGGCAACGCTACCTTTGCCATGTTTC |
| | | | AGAAACAACTCTGGCGCATCGGGCTTCCCATACAA |
| | | | TCGATAGATTGTCGCACCTGATTGCCCGACATTATC |
| | | | GCGAGCCCATTTATACCCATATAAATCAGCATCCA |
| | | | TGTTGGAATTTAATCGCGGCCTAGAGCAAGACGTT |
| | | | TCCCGTTGAATATGGCTCATACTCTTCCTTTTTCAA |
| | | | TATTATTGAAGCATTTATCAGGGTTATTGTCTCATG |
| | | | AGCGGATACATATTTGAATGTATTTAGAAAAATAA |
| | | | ACAAATAGGGGTTCCGCGCACATTTCCCCGAAAAG |
| | | | TGCCACCTGACGTCTAAGAAACCATTATTATCATG |
| | | | ACATTAACCTATAAAAATAGGCGTATCACGAGGCC |
| | | | CTTTCGTC |
| 71 | pYZZ460 | Rep2Cap2-p5p5 | TCGCGCGTTTCGGTGATGACGGTGAAAACCTCTGA |
| | | | CACATGCAGCTCCCGGAGACGGTCACAGCTTGTCT |
| | | | GTAAGCGGATGCCGGGAGCAGACAAGCCCGTCAG |
| | | | GGCGCGTCAGCGGGTGTTGGCGGGTGTCGGGGCTG |
| | | | GCTTAACTATGCGGCATCAGAGCAGATTGTACTGA |
| | | | GAGTGCACCATATGCGGTGTGAAATACCGCACAGA |
| | | | TGCGTAAGGAGAAAATACCGCATCAGGCGCCATTC |
| | | | GCCATTCAGGCTGCGCAACTGTTGGGAAGGGCGAT |
| | | | CGGTGCGGGCCTCTTCGCTATTACGCCAGCTGGCG |
| | | | AAAGGGGGATGTGCTGCAAGGCGATTAAGTTGGGT |
| | | | AACGCCAGGGTTTTCCCAGTCACGACGTTGTAAAA |
| | | | CGACGGCCAGTGAATTGACGCGTATTGGGATCGCG |
| | | | CAGCCGCCATGCCGGGGTTTTACGAGATTGTGATT |
| | | | AAGGTCCCCAGCGACCTTGACGAGCATCTGCCCGG |
| | | | CATTTCTGACAGCTTTGTGAACTGGGTGGCCGAGA |
| | | | AGGAATGGGAGTTGCCGCCAGATTCTGACATGGAT |
| | | | CTGAATCTGATTGAGCAGGCACCCCTGACCGTGGC |
| | | | CGAGAAGCTGCAGCGCGACTTTCTGACGGAATGGC |
| | | | GCCGTGTGAGTAAGGCCCCGGAGGCTCTTTTCTTTG |
| | | | TGCAATTTGAGAAGGGAGAGAGCTACTTCCACATG |
| | | | CACGTGCTCGTGGAAACCACCGGGGTGAAATCCAT |
| | | | GGTTTTGGGACGTTTCCTGAGTCAGATTCGCGAAA |
| | | | AACTGATTCAGAGAATTTACCGCGGGATCGAGCCG |
| | | | ACTTTGCCAAACTGGTTCGCGGTCACAAAGACCAG |
| | | | AAATGGCGCCGGAGGCGGGAACAAGGTGGTGGAT |
| | | | GAGTGCTACATCCCCAATTACTTGCTCCCCAAAACC |
| | | | CAGCCTGAGCTCCAGTGGGCGTGGACTAATATGGA |
| | | | ACAGTATTTAAGCGCCTGTTTGAATCTCACGGAGC |
| | | | GTAAACGGTTGGTGGCGCAGCATCTGACGCACGTG |
| | | | TCGCAGACGCAGGAGCAGAACAAAGAGAATCAGA |
| | | | ATCCCAATTCTGATGCGCCGGTGATCAGATCAAAA |
| | | | ACTTCAGCCAGGTACATGGAGCTGGTCGGGTGGCT |
| | | | CGTGGACAAGGGGATTACCTCGGAGAAGCAGTGG |
| | | | ATCCAGGAGGACCAGGCCTCATACATCTCCTTCAA |
| | | | TGCGGCCTCCAACTCGCGGTCCCAAATCAAGGCTG |
| | | | CCTTGGACAATGCGGGAAAGATTATGAGCCTGACT |
| | | | AAAACCGCCCCCGACTACCTGGTGGGCCAGCAGCC |
| | | | CGTGGAGGACATTTCCAGCAATCGGATTTATAAAA |
| | | | TTTTGGAACTAAACGGGTACGATCCCCAATATGCG |
| | | | GCTTCCGTCTTTCTGGGATGGGCCACGAAAAAGTT |
| | | | CGGCAAGAGGAACACCATCTGGCTGTTTGGGCCTG |
| | | | CAACTACCGGGAAGACCAACATCGCGGAGGCCATA |
| | | | GCCCACACTGTGCCCTTCTACGGGTGCGTAAACTG |
| | | | GACCAATGAGAACTTTCCCTTCAACGACTGTGTCG |
| | | | ACAAGATGGTGATCTGGTGGGAGGAGGGGAAGAT |
| | | | GACCGCCAAGGTCGTGGAGTCGGCCAAAGCCATTC |
| | | | TCGGAGGAAGCAAGGTGCGCGTGGACCAGAAATG |
| | | | CAAGTCCTCGGCCCAGATAGACCCGACTCCCGTGA |
| | | | TCGTCACCTCCAACACCAACATGTGCGCCGTGATT |
| | | | GACGGGAACTCAACGACCTTCGAACACCAGCAGCC |
| | | | GTTGCAAGACCGGATGTTCAAATTTGAACTCACCC |
| | | | GCCGTCTGGATCATGACTTTGGGAAGGTCACCAAG |
| | | | CAGGAAGTCAAAGACTTTTTCCGGTGGGCAAAGGA |
| | | | TCACGTGGTTGAGGTGGAGCATGAATTCTACGTCA |
| | | | AAAAGGGTGGAGCCAAGAAAAGACCCGCCCCCAG |
| | | | TGACGCAGATATAAGTGAGCCCAAACGGGTGCGCG |
| | | | AGTCAGTTGCGCAGCCATCGACGTCAGACGCGGAA |
| | | | GCTTCGATCAACTACGCAGACAGGTACCAAAACAA |
| | | | ATGTTCTCGTCACGTGGGCATGAATCTGATGCTGTT |
| | | | TCCCTGCAGACAATGCGAGAGAATGAATCAGAATT |
| | | | CAAATATCTGCTTCACTCACGGACAGAAAGACTGT |
| | | | TTAGAGTGCTTTCCCGTGTCAGAATCTCAACCCGTT |
| | | | TCTGTCGTCAAAAAGGCGTATCAGAAACTGTGCTA |
| | | | CATTCATCATATCATGGGAAAGGTGCCAGACGCTT |
| | | | GCACTGCCTGCGATCTGGTCAATGTGGATTTGGAT |
| | | | GACTGCATCTTTGAACAATAAATGATTTAAATCAG |
| | | | GTatggctgccgatggttatcttccagattggctcgaggacactctctctgaaggaat |
| | | | aagacagtggtggaagctcaaacctggcccaccaccaccaaagcccgcagagcg |
| | | | gcataaggacgacagcaggggtcttgtgcttcctgggtacaagtacctcggaccctt |
| | | | caacggactcgacaagggagagccggtcaacgaggcagacgccgcggccctcg |
| | | | agcacgacaaagcctacgaccggcagctcgacagcggagacaacccgtacctca |
| | | | agtacaaccacgccgacgcggagtttcaggagcgccttaaagaagatacgtcttttg |
| | | | ggggcaacctcggacgagcagtcttccaggcgaaaaagagggttcttgaacctctg |
| | | | ggcctggttgaggaacctgttaagacggctccgggaaaaaagaggccggtagagc |
| | | | actctcctgtggagccagactcctcctcgggaaccggaaaggcgggccagcagcc |
| | | | tgcaagaaaaagattgaattttggtcagactggagacgcagactcagtacctgaccc |
| | | | ccagcctctcggacagccaccagcagccccctctggtctgggaactaatacgatgg |
| | | | ctacaggcagtggcgcaccaatggcagacaataacgagggcgccgacggagtgg |
| | | | gtaattcctcgggaaattggcattgcgattccacatggatgggcgacagagtcatcac |
| | | | caccagcacccgaacctgggccctgcccacctacaacaaccacctctacaaacaaa |
| | | | tttccagccaatcaggagcctcgaacgacaatcactactttggctacagcaccccttg |
| | | | ggggtattttgacttcaacagattccactgccacttttcaccacgtgactggcaaagac |
| | | | tcatcaacaacaactggggattccgacccaagagactcaacttcaagctctttaacatt |
| | | | caagtcaaagaggtcacgcagaatgacggtacgacgacgattgccaataaccttac |
| | | | cagcacggttcaggtgtttactgactcggagtaccagctcccgtacgtcctcggctcg |
| | | | gcgcatcaaggatgcctcccgccgttcccagcagacgtcttcatggtgccacagtat |
| | | | ggatacctcaccctgaacaacgggagtcaggcagtaggacgctcttcattttactgcc |
| | | | tggagtactttccttctcagatgctgcgtaccggaaacaactttaccttcagctacacttt |
| | | | tgaggacgttcctttccacagcagctacgctcacagccagagtctggaccgtctcatg |
| | | | aatcctctcatcgaccagtacctgtattacttgagcagaacaaacactccaagtggaa |
| | | | ccaccacgcagtcaaggcttcagttttctcaggccggagcgagtgacattcgggacc |
| | | | agtctaggaactggcttcctggaccctgttaccgccagcagcgagtatcaaagacat |
| | | | ctgcggataacaacaacagtgaatactcgtggactggagctaccaagtaccacctca |
| | | | atggcagagactctctggtgaatccgggcccggccatggcaagccacaaggacga |
| | | | tgaagaaaagttttttcctcagagcggggttctcatctttgggaagcaaggctcagag |
| | | | aaaacaaatgtggacattgaaaaggtcatgattacagacgaagaggaaatcaggac |
| | | | aaccaatcccgtggctacggagcagtatggttctgtatctaccaacctccagagagg |
| | | | caacagacaagcagctaccgcagatgtcaacacacaaggcgttcttccaggcatgg |
| | | | tctggcaggacagagatgtgtaccttcaggggcccatctgggcaaagattccacaca |
| | | | cggacggacattttcacccctctcccctcatgggtggattcggacttaaacaccctcct |
| | | | ccacagattctcatcaagaacaccccggtacctgcgaatccttcgaccaccttcagtg |
| | | | cggcaaagtttgcttccttcatcacacagtactccacgggacaggtcagcgtggaga |
| | | | tcgagtgggagctgcagaaggaaaacagcaaacgctggaatcccgaaattcagta |
| | | | cacttccaactacaacaagtctgttaatgtggactttactgtggacactaatggcgtgta |
| | | | ttcagagcctcgccccattggcaccagatacctgactcgtaatctgtaaTTGCTT |
| | | | GTTAATCAATAAACCGTTTAATTCGTTTCAGTTGAA |
| | | | CTTTGGTCTCTGCCCTAGGGGAGGTCCTGTATTAGA |
| | | | GGTCACGTGAGTGTTTTGCGACATTTTGCGACACCA |
| | | | TGTGGTCACGCTGGGTATTTAAGCCCGAGTGAGCA |
| | | | CGCAGGGTCTCCATTTTGAAGCGGGAGGTTTGAAC |
| | | | ATACAGACGAAACTAGTTGGAGATTATCGAGCATT |
| | | | CTATCACGTCGACGACCACTAGTGAGTTACTAGAG |
| | | | CCGAGGGGTAACGTTGATGCTCCTAAAAAgaggtcctgt |
| | | | attagaggtcacgtgagtgttttgcgacattttgcgacaccatgtggtcacgctgggta |
| | | | tttaagcccgagtgagcacgcagggtctccattttgaagcgggaggtttgaaATC |
| | | | CCAATGGCGCGCCGAGCTTGGCTCGAGCATGGTCA |
| | | | TAGCTGTTTCCTGTGTGAAATTGTTATCCGCTCACA |
| | | | ATTCCACACAACATACGAGCCGGAAGCATAAAGTG |
| | | | TAAAGCCTGGGGTGCCTAATGAGTGAGCTAACTCA |
| | | | CATTAATTGCGTTGCGCTCACTGCCCGCTTTCCAGT |
| | | | CGGGAAACCTGTCGTGCCAGCTGCATTAATGAATC |
| | | | GGCCAACGCGCGGGGAGAGGCGGTTTGCGTATTGG |
| | | | GCGCTCTTCCGCTTCCTCGCTCACTGACTCGCTGCG |
| | | | CTCGGTCGTTCGGCTGCGGCGAGCGGTATCAGCTC |
| | | | ACTCAAAGGCGGTAATACGGTTATCCACAGAATCA |
| | | | GGGGATAACGCAGGAAAGAACATGTGAGCAAAAG |
| | | | GCCAGCAAAAGGCCAGGAACCGTAAAAAGGCCGC |
| | | | GTTGCTGGCGTTTTTCCATAGGCTCCGCCCCCCTGA |
| | | | CGAGCATCACAAAAATCGACGCTCAAGTCAGAGGT |
| | | | GGCGAAACCCGACAGGACTATAAAGATACCAGGC |
| | | | GTTTCCCCCTGGAAGCTCCCTCGTGCGCTCTCCTGT |
| | | | TCCGACCCTGCCGCTTACCGGATACCTGTCCGCCTT |
| | | | TCTCCCTTCGGGAAGCGTGGCGCTTTCTCATAGCTC |
| | | | ACGCTGTAGGTATCTCAGTTCGGTGTAGGTCGTTCG |
| | | | CTCCAAGCTGGGCTGTGTGCACGAACCCCCCGTTC |
| | | | AGCCCGACCGCTGCGCCTTATCCGGTAACTATCGTC |
| | | | TTGAGTCCAACCCGGTAAGACACGACTTATCGCCA |
| | | | CTGGCAGCAGCCACTGGTAACAGGATTAGCAGAGC |
| | | | GAGGTATGTAGGCGGTGCTACAGAGTTCTTGAAGT |
| | | | GGTGGCCTAACTACGGCTACACTAGAAGAACAGTA |
| | | | TTTGGTATCTGCGCTCTGCTGAAGCCAGTTACCTTC |
| | | | GGAAAAAGAGTTGGTAGCTCTTGATCCGGCAAACA |
| | | | AACCACCGCTGGTAGCGGTGGTTTTTTTGTTTGCAA |
| | | | GCAGCAGATTACGCGCAGAAAAAAAGGATCTCAA |
| | | | GAAGATCCTTTGATCTTTTCTACGGGGTCTGACGCT |
| | | | CAGTGGAACGAAAACTCACGTTAAGGGATTTTGGT |
| | | | CATGAGATTATCAAAAAGGATCTTCACCTAGATCC |
| | | | TTTTAAATTAAAAATGAAGTTTTAAATCAATCTAAA |
| | | | GTATATATGAGTAAACTTGGTCTGACAGTTAGAAA |
| | | | AACTCATCGAGCATCAAATGAAACTGCAATTTATT |
| | | | CATATCAGGATTATCAATACCATATTTTTGAAAAA |
| | | | GCCGTTTCTGTAATGAAGGAGAAAACTCACCGAGG |
| | | | CAGTTCCATAGGATGGCAAGATCCTGGTATCGGTC |
| | | | TGCGATTCCGACTCGTCCAACATCAATACAACCTAT |
| | | | TAATTTCCCCTCGTCAAAAATAAGGTTATCAAGTG |
| | | | AGAAATCACCATGAGTGACGACTGAATCCGGTGAG |
| | | | AATGGCAAAAGTTTATGCATTTCTTTCCAGACTTGT |
| | | | TCAACAGGCCAGCCATTACGCTCGTCATCAAAATC |
| | | | ACTCGCATCAACCAAACCGTTATTCATTCGTGATTG |
| | | | CGCCTGAGCGAGACGAAATACGCGATCGCTGTTAA |
| | | | AAGGACAATTACAAACAGGAATCGAATGCAACCG |
| | | | GCGCAGGAACACTGCCAGCGCATCAACAATATTTT |
| | | | CACCTGAATCAGGATATTCTTCTAATACCTGGAATG |
| | | | CTGTTTTCCCAGGGATCGCAGTGGTGAGTAACCAT |
| | | | GCATCATCAGGAGTACGGATAAAATGCTTGATGGT |
| | | | CGGAAGAGGCATAAATTCCGTCAGCCAGTTTAGTC |
| | | | TGACCATCTCATCTGTAACATCATTGGCAACGCTAC |
| | | | CTTTGCCATGTTTCAGAAACAACTCTGGCGCATCGG |
| | | | GCTTCCCATACAATCGATAGATTGTCGCACCTGATT |
| | | | GCCCGACATTATCGCGAGCCCATTTATACCCATATA |
| | | | AATCAGCATCCATGTTGGAATTTAATCGCGGCCTA |
| | | | GAGCAAGACGTTTCCCGTTGAATATGGCTCATACT |
| | | | CTTCCTTTTTCAATATTATTGAAGCATTTATCAGGG |
| | | | TTATTGTCTCATGAGCGGATACATATTTGAATGTAT |
| | | | TTAGAAAAATAAACAAATAGGGGTTCCGCGCACAT |
| | | | TTCCCCGAAAAGTGCCACCTGACGTCTAAGAAACC |
| | | | ATTATTATCATGACATTAACCTATAAAAATAGGCG |
| | | | TATCACGAGGCCCTTTCGTC |
| 72 | pYZZ461 | Rep2Cap2-p5p5 | TCGCGCGTTTCGGTGATGACGGTGAAAACCTCTGA |
| | | | CACATGCAGCTCCCGGAGACGGTCACAGCTTGTCT |
| | | | GTAAGCGGATGCCGGGAGCAGACAAGCCCGTCAG |
| | | | GGCGCGTCAGCGGGTGTTGGCGGGTGTCGGGGCTG |
| | | | GCTTAACTATGCGGCATCAGAGCAGATTGTACTGA |
| | | | GAGTGCACCATATGCGGTGTGAAATACCGCACAGA |
| | | | TGCGTAAGGAGAAAATACCGCATCAGGCGCCATTC |
| | | | GCCATTCAGGCTGCGCAACTGTTGGGAAGGGCGAT |
| | | | CGGTGCGGGCCTCTTCGCTATTACGCCAGCTGGCG |
| | | | AAAGGGGGATGTGCTGCAAGGCGATTAAGTTGGGT |
| | | | AACGCCAGGGTTTTCCCAGTCACGACGTTGTAAAA |
| | | | CGACGGCCAGTGAATTGACGCGTATTGGGATCGCG |
| | | | CAGCCGCCATGCCGGGGTTTTACGAGATTGTGATT |
| | | | AAGGTCCCCAGCGACCTTGACGAGCATCTGCCCGG |
| | | | CATTTCTGACAGCTTTGTGAACTGGGTGGCCGAGA |
| | | | AGGAATGGGAGTTGCCGCCAGATTCTGACATGGAT |
| | | | CTGAATCTGATTGAGCAGGCACCCCTGACCGTGGC |
| | | | CGAGAAGCTGCAGCGCGACTTTCTGACGGAATGGC |
| | | | GCCGTGTGAGTAAGGCCCCGGAGGCTCTTTTCTTTG |
| | | | TGCAATTTGAGAAGGGAGAGAGCTACTTCCACATG |
| | | | CACGTGCTCGTGGAAACCACCGGGGTGAAATCCAT |
| | | | GGTTTTGGGACGTTTCCTGAGTCAGATTCGCGAAA |
| | | | AACTGATTCAGAGAATTTACCGCGGGATCGAGCCG |
| | | | ACTTTGCCAAACTGGTTCGCGGTCACAAAGACCAG |
| | | | AAATGGCGCCGGAGGCGGGAACAAGGTGGTGGAT |
| | | | GAGTGCTACATCCCCAATTACTTGCTCCCCAAAACC |
| | | | CAGCCTGAGCTCCAGTGGGCGTGGACTAATATGGA |
| | | | ACAGTATTTAAGCGCCTGTTTGAATCTCACGGAGC |
| | | | GTAAACGGTTGGTGGCGCAGCATCTGACGCACGTG |
| | | | TCGCAGACGCAGGAGCAGAACAAAGAGAATCAGA |
| | | | ATCCCAATTCTGATGCGCCGGTGATCAGATCAAAA |
| | | | ACTTCAGCCAGGTACATGGAGCTGGTCGGGTGGCT |
| | | | CGTGGACAAGGGGATTACCTCGGAGAAGCAGTGG |
| | | | ATCCAGGAGGACCAGGCCTCATACATCTCCTTCAA |
| | | | TGCGGCCTCCAACTCGCGGTCCCAAATCAAGGCTG |
| | | | CCTTGGACAATGCGGGAAAGATTATGAGCCTGACT |
| | | | AAAACCGCCCCCGACTACCTGGTGGGCCAGCAGCC |
| | | | CGTGGAGGACATTTCCAGCAATCGGATTTATAAAA |
| | | | TTTTGGAACTAAACGGGTACGATCCCCAATATGCG |
| | | | GCTTCCGTCTTTCTGGGATGGGCCACGAAAAAGTT |
| | | | CGGCAAGAGGAACACCATCTGGCTGTTTGGGCCTG |
| | | | CAACTACCGGGAAGACCAACATCGCGGAGGCCATA |
| | | | GCCCACACTGTGCCCTTCTACGGGTGCGTAAACTG |
| | | | GACCAATGAGAACTTTCCCTTCAACGACTGTGTCG |
| | | | ACAAGATGGTGATCTGGTGGGAGGAGGGGAAGAT |
| | | | GACCGCCAAGGTCGTGGAGTCGGCCAAAGCCATTC |
| | | | TCGGAGGAAGCAAGGTGCGCGTGGACCAGAAATG |
| | | | CAAGTCCTCGGCCCAGATAGACCCGACTCCCGTGA |
| | | | TCGTCACCTCCAACACCAACATGTGCGCCGTGATT |
| | | | GACGGGAACTCAACGACCTTCGAACACCAGCAGCC |
| | | | GTTGCAAGACCGGATGTTCAAATTTGAACTCACCC |
| | | | GCCGTCTGGATCATGACTTTGGGAAGGTCACCAAG |
| | | | CAGGAAGTCAAAGACTTTTTCCGGTGGGCAAAGGA |
| | | | TCACGTGGTTGAGGTGGAGCATGAATTCTACGTCA |
| | | | AAAAGGGTGGAGCCAAGAAAAGACCCGCCCCCAG |
| | | | TGACGCAGATATAAGTGAGCCCAAACGGGTGCGCG |
| | | | AGTCAGTTGCGCAGCCATCGACGTCAGACGCGGAA |
| | | | GCTTCGATCAACTACGCAGACAGGTACCAAAACAA |
| | | | ATGTTCTCGTCACGTGGGCATGAATCTGATGCTGTT |
| | | | TCCCTGCAGACAATGCGAGAGAATGAATCAGAATT |
| | | | CAAATATCTGCTTCACTCACGGACAGAAAGACTGT |
| | | | TTAGAGTGCTTTCCCGTGTCAGAATCTCAACCCGTT |
| | | | TCTGTCGTCAAAAAGGCGTATCAGAAACTGTGCTA |
| | | | CATTCATCATATCATGGGAAAGGTGCCAGACGCTT |
| | | | GCACTGCCTGCGATCTGGTCAATGTGGATTTGGAT |
| | | | GACTGCATCTTTGAACAATAAATGATTTAAATCAG |
| | | | GTatggctgccgatggttatcttccagattggctcgaggacactctctctgaaggaat |
| | | | aagacagtggtggaagctcaaacctggcccaccaccaccaaagcccgcagagcg |
| | | | gcataaggacgacagcaggggtcttgtgcttcctgggtacaagtacctcggaccctt |
| | | | caacggactcgacaagggagagccggtcaacgaggcagacgccgcggccctcg |
| | | | agcacgacaaagcctacgaccggcagctcgacagcggagacaacccgtacctca |
| | | | agtacaaccacgccgacgcggagtttcaggagcgccttaaagaagatacgtcttttg |
| | | | ggggcaacctcggacgagcagtcttccaggcgaaaaagagggttcttgaacctctg |
| | | | ggcctggttgaggaacctgttaagacggctccgggaaaaaagaggccggtagagc |
| | | | actctcctgtggagccagactcctcctcgggaaccggaaagggggccagcagcc |
| | | | tgcaagaaaaagattgaattttggtcagactggagacgcagactcagtacctgaccc |
| | | | ccagcctctcggacagccaccagcagccccctctggtctgggaactaatacgatgg |
| | | | ctacaggcagtggcgcaccaatggcagacaataacgagggcgccgacggagtgg |
| | | | gtaattcctcgggaaattggcattgcgattccacatggatgggcgacagagtcatcac |
| | | | caccagcacccgaacctgggccctgcccacctacaacaaccacctctacaaacaaa |
| | | | tttccagccaatcaggagcctcgaacgacaatcactactttggctacagcaccccttg |
| | | | ggggtattttgacttcaacagattccactgccacttttcaccacgtgactggcaaagac |
| | | | tcatcaacaacaactggggattccgacccaagagactcaacttcaagctctttaacatt |
| | | | caagtcaaagaggtcacgcagaatgacggtacgacgacgattgccaataaccttac |
| | | | cagcacggttcaggtgtttactgactcggagtaccagctcccgtacgtcctcggctcg |
| | | | gcgcatcaaggatgcctcccgccgttcccagcagacgtcttcatggtgccacagtat |
| | | | ggatacctcaccctgaacaacgggagtcaggcagtaggacgctcttcattttactgcc |
| | | | tggagtactttccttctcagatgctgcgtaccggaaacaactttaccttcagctacacttt |
| | | | tgaggacgttcctttccacagcagctacgctcacagccagagtctggaccgtctcatg |
| | | | aatcctctcatcgaccagtacctgtattacttgagcagaacaaacactccaagtggaa |
| | | | ccaccacgcagtcaaggcttcagttttctcaggccggagcgagtgacattcgggacc |
| | | | agtctaggaactggcttcctggaccctgttaccgccagcagcgagtatcaaagacat |
| | | | ctgcggataacaacaacagtgaatactcgtggactggagctaccaagtaccacctca |
| | | | atggcagagactctctggtgaatccgggcccggccatggcaagccacaaggacga |
| | | | tgaagaaaagttttttcctcagagcggggttctcatctttgggaagcaaggctcagag |
| | | | aaaacaaatgtggacattgaaaaggtcatgattacagacgaagaggaaatcaggac |
| | | | aaccaatcccgtggctacggagcagtatggttctgtatctaccaacctccagagagg |
| | | | caacagacaagcagctaccgcagatgtcaacacacaaggcgttcttccaggcatgg |
| | | | tctggcaggacagagatgtgtaccttcaggggcccatctgggcaaagattccacaca |
| | | | cggacggacattttcacccctctcccctcatgggtggattcggacttaaacaccctcct |
| | | | ccacagattctcatcaagaacaccccggtacctgcgaatccttcgaccaccttcagtg |
| | | | cggcaaagtttgcttccttcatcacacagtactccacgggacaggtcagcgtggaga |
| | | | tcgagtgggagctgcagaaggaaaacagcaaacgctggaatcccgaaattcagta |
| | | | cacttccaactacaacaagtctgttaatgtggactttactgtggacactaatggcgtgta |
| | | | ttcagagcctcgccccattggcaccagatacctgactcgtaatctgtaaTTGCTT |
| | | | GTTAATCAATAAACCGTTTAATTCGTTTCAGTTGAA |
| | | | CTTTGGTCTCTGCCCTAGGGGAGGTCCTGTATTAGA |
| | | | GGTCACGTGAGTGTTTTGCGACATTTTGCGACACCA |
| | | | TGTGGTCACGCTGGGTATTTAAGCCCGAGTGAGCA |
| | | | CGCAGGGTCTCCATTTTGAAGCGGGAGGTTTGAAC |
| | | | ATACAGACGAAACTAGTTGGAGATTATCGAGCATT |
| | | | CTATCACGTCGACGACCACTAGTGAGTTACTAGAG |
| | | | CCGAGGGGTAACGTTGATGCTCCTAAAAATCTCTC |
| | | | GGTCGATGCAAGCGATTACACTCCTGTCACATCAT |
| | | | AATCGTTTGCTATTCAGGGCTTGATCAACACTGGAT |
| | | | TGCTTTTCACTTAAAGTATTATGgaggtcctgtattagaggtcac |
| | | | gtgagtgttttgcgacattttgcgacaccatgtggtcacgctgggtatttaagcccgag |
| | | | tgagcacgcagggtctccattttgaagcgggaggtttgaaATCCCAATGGC |
| | | | GCGCCGAGCTTGGCTCGAGCATGGTCATAGCTGTT |
| | | | TCCTGTGTGAAATTGTTATCCGCTCACAATTCCACA |
| | | | CAACATACGAGCCGGAAGCATAAAGTGTAAAGCCT |
| | | | GGGGTGCCTAATGAGTGAGCTAACTCACATTAATT |
| | | | GCGTTGCGCTCACTGCCCGCTTTCCAGTCGGGAAA |
| | | | CCTGTCGTGCCAGCTGCATTAATGAATCGGCCAAC |
| | | | GCGCGGGGAGAGGCGGTTTGCGTATTGGGCGCTCT |
| | | | TCCGCTTCCTCGCTCACTGACTCGCTGCGCTCGGTC |
| | | | GTTCGGCTGCGGCGAGCGGTATCAGCTCACTCAAA |
| | | | GGCGGTAATACGGTTATCCACAGAATCAGGGGATA |
| | | | ACGCAGGAAAGAACATGTGAGCAAAAGGCCAGCA |
| | | | AAAGGCCAGGAACCGTAAAAAGGCCGCGTTGCTG |
| | | | GCGTTTTTCCATAGGCTCCGCCCCCCTGACGAGCAT |
| | | | CACAAAAATCGACGCTCAAGTCAGAGGTGGCGAA |
| | | | ACCCGACAGGACTATAAAGATACCAGGCGTTTCCC |
| | | | CCTGGAAGCTCCCTCGTGCGCTCTCCTGTTCCGACC |
| | | | CTGCCGCTTACCGGATACCTGTCCGCCTTTCTCCCT |
| | | | TCGGGAAGCGTGGCGCTTTCTCATAGCTCACGCTGT |
| | | | AGGTATCTCAGTTCGGTGTAGGTCGTTCGCTCCAAG |
| | | | CTGGGCTGTGTGCACGAACCCCCCGTTCAGCCCGA |
| | | | CCGCTGCGCCTTATCCGGTAACTATCGTCTTGAGTC |
| | | | CAACCCGGTAAGACACGACTTATCGCCACTGGCAG |
| | | | CAGCCACTGGTAACAGGATTAGCAGAGCGAGGTAT |
| | | | GTAGGCGGTGCTACAGAGTTCTTGAAGTGGTGGCC |
| | | | TAACTACGGCTACACTAGAAGAACAGTATTTGGTA |
| | | | TCTGCGCTCTGCTGAAGCCAGTTACCTTCGGAAAA |
| | | | AGAGTTGGTAGCTCTTGATCCGGCAAACAAACCAC |
| | | | CGCTGGTAGCGGTGGTTTTTTTGTTTGCAAGCAGCA |
| | | | GATTACGCGCAGAAAAAAAGGATCTCAAGAAGAT |
| | | | CCTTTGATCTTTTCTACGGGGTCTGACGCTCAGTGG |
| | | | AACGAAAACTCACGTTAAGGGATTTTGGTCATGAG |
| | | | ATTATCAAAAAGGATCTTCACCTAGATCCTTTTAAA |
| | | | TTAAAAATGAAGTTTTAAATCAATCTAAAGTATAT |
| | | | ATGAGTAAACTTGGTCTGACAGTTAGAAAAACTCA |
| | | | TCGAGCATCAAATGAAACTGCAATTTATTCATATC |
| | | | AGGATTATCAATACCATATTTTTGAAAAAGCCGTTT |
| | | | CTGTAATGAAGGAGAAAACTCACCGAGGCAGTTCC |
| | | | ATAGGATGGCAAGATCCTGGTATCGGTCTGCGATT |
| | | | CCGACTCGTCCAACATCAATACAACCTATTAATTTC |
| | | | CCCTCGTCAAAAATAAGGTTATCAAGTGAGAAATC |
| | | | ACCATGAGTGACGACTGAATCCGGTGAGAATGGCA |
| | | | AAAGTTTATGCATTTCTTTCCAGACTTGTTCAACAG |
| | | | GCCAGCCATTACGCTCGTCATCAAAATCACTCGCA |
| | | | TCAACCAAACCGTTATTCATTCGTGATTGCGCCTGA |
| | | | GCGAGACGAAATACGCGATCGCTGTTAAAAGGACA |
| | | | ATTACAAACAGGAATCGAATGCAACCGGCGCAGG |
| | | | AACACTGCCAGCGCATCAACAATATTTTCACCTGA |
| | | | ATCAGGATATTCTTCTAATACCTGGAATGCTGTTTT |
| | | | CCCAGGGATCGCAGTGGTGAGTAACCATGCATCAT |
| | | | CAGGAGTACGGATAAAATGCTTGATGGTCGGAAGA |
| | | | GGCATAAATTCCGTCAGCCAGTTTAGTCTGACCATC |
| | | | TCATCTGTAACATCATTGGCAACGCTACCTTTGCCA |
| | | | TGTTTCAGAAACAACTCTGGCGCATCGGGCTTCCC |
| | | | ATACAATCGATAGATTGTCGCACCTGATTGCCCGA |
| | | | CATTATCGCGAGCCCATTTATACCCATATAAATCAG |
| | | | CATCCATGTTGGAATTTAATCGCGGCCTAGAGCAA |
| | | | GACGTTTCCCGTTGAATATGGCTCATACTCTTCCTT |
| | | | TTTCAATATTATTGAAGCATTTATCAGGGTTATTGT |
| | | | CTCATGAGCGGATACATATTTGAATGTATTTAGAA |
| | | | AAATAAACAAATAGGGGTTCCGCGCACATTTCCCC |
| | | | GAAAAGTGCCACCTGACGTCTAAGAAACCATTATT |
| | | | ATCATGACATTAACCTATAAAAATAGGCGTATCAC |
| | | | GAGGCCCTTTCGTC |
| 73 | pYZZ462 | Rep2Cap2-p5p5 | TCGCGCGTTTCGGTGATGACGGTGAAAACCTCTGA |
| | | | CACATGCAGCTCCCGGAGACGGTCACAGCTTGTCT |
| | | | GTAAGCGGATGCCGGGAGCAGACAAGCCCGTCAG |
| | | | GGCGCGTCAGCGGGTGTTGGCGGGTGTCGGGGCTG |
| | | | GCTTAACTATGCGGCATCAGAGCAGATTGTACTGA |
| | | | GAGTGCACCATATGCGGTGTGAAATACCGCACAGA |
| | | | TGCGTAAGGAGAAAATACCGCATCAGGCGCCATTC |
| | | | GCCATTCAGGCTGCGCAACTGTTGGGAAGGGCGAT |
| | | | CGGTGCGGGCCTCTTCGCTATTACGCCAGCTGGCG |
| | | | AAAGGGGGATGTGCTGCAAGGCGATTAAGTTGGGT |
| | | | AACGCCAGGGTTTTCCCAGTCACGACGTTGTAAAA |
| | | | CGACGGCCAGTGAATTGACGCGTATTGGGATCGCG |
| | | | CAGCCGCCATGCCGGGGTTTTACGAGATTGTGATT |
| | | | AAGGTCCCCAGCGACCTTGACGAGCATCTGCCCGG |
| | | | CATTTCTGACAGCTTTGTGAACTGGGTGGCCGAGA |
| | | | AGGAATGGGAGTTGCCGCCAGATTCTGACATGGAT |
| | | | CTGAATCTGATTGAGCAGGCACCCCTGACCGTGGC |
| | | | CGAGAAGCTGCAGCGCGACTTTCTGACGGAATGGC |
| | | | GCCGTGTGAGTAAGGCCCCGGAGGCTCTTTTCTTTG |
| | | | TGCAATTTGAGAAGGGAGAGAGCTACTTCCACATG |
| | | | CACGTGCTCGTGGAAACCACCGGGGTGAAATCCAT |
| | | | GGTTTTGGGACGTTTCCTGAGTCAGATTCGCGAAA |
| | | | AACTGATTCAGAGAATTTACCGCGGGATCGAGCCG |
| | | | ACTTTGCCAAACTGGTTCGCGGTCACAAAGACCAG |
| | | | AAATGGCGCCGGAGGCGGGAACAAGGTGGTGGAT |
| | | | GAGTGCTACATCCCCAATTACTTGCTCCCCAAAACC |
| | | | CAGCCTGAGCTCCAGTGGGCGTGGACTAATATGGA |
| | | | ACAGTATTTAAGCGCCTGTTTGAATCTCACGGAGC |
| | | | GTAAACGGTTGGTGGCGCAGCATCTGACGCACGTG |
| | | | TCGCAGACGCAGGAGCAGAACAAAGAGAATCAGA |
| | | | ATCCCAATTCTGATGCGCCGGTGATCAGATCAAAA |
| | | | ACTTCAGCCAGGTACATGGAGCTGGTCGGGTGGCT |
| | | | CGTGGACAAGGGGATTACCTCGGAGAAGCAGTGG |
| | | | ATCCAGGAGGACCAGGCCTCATACATCTCCTTCAA |
| | | | TGCGGCCTCCAACTCGCGGTCCCAAATCAAGGCTG |
| | | | CCTTGGACAATGCGGGAAAGATTATGAGCCTGACT |
| | | | AAAACCGCCCCCGACTACCTGGTGGGCCAGCAGCC |
| | | | CGTGGAGGACATTTCCAGCAATCGGATTTATAAAA |
| | | | TTTTGGAACTAAACGGGTACGATCCCCAATATGCG |
| | | | GCTTCCGTCTTTCTGGGATGGGCCACGAAAAAGTT |
| | | | CGGCAAGAGGAACACCATCTGGCTGTTTGGGCCTG |
| | | | CAACTACCGGGAAGACCAACATCGCGGAGGCCATA |
| | | | GCCCACACTGTGCCCTTCTACGGGTGCGTAAACTG |
| | | | GACCAATGAGAACTTTCCCTTCAACGACTGTGTCG |
| | | | ACAAGATGGTGATCTGGTGGGAGGAGGGGAAGAT |
| | | | GACCGCCAAGGTCGTGGAGTCGGCCAAAGCCATTC |
| | | | TCGGAGGAAGCAAGGTGCGCGTGGACCAGAAATG |
| | | | CAAGTCCTCGGCCCAGATAGACCCGACTCCCGTGA |
| | | | TCGTCACCTCCAACACCAACATGTGCGCCGTGATT |
| | | | GACGGGAACTCAACGACCTTCGAACACCAGCAGCC |
| | | | GTTGCAAGACCGGATGTTCAAATTTGAACTCACCC |
| | | | GCCGTCTGGATCATGACTTTGGGAAGGTCACCAAG |
| | | | CAGGAAGTCAAAGACTTTTTCCGGTGGGCAAAGGA |
| | | | TCACGTGGTTGAGGTGGAGCATGAATTCTACGTCA |
| | | | AAAAGGGTGGAGCCAAGAAAAGACCCGCCCCCAG |
| | | | TGACGCAGATATAAGTGAGCCCAAACGGGTGCGCG |
| | | | AGTCAGTTGCGCAGCCATCGACGTCAGACGCGGAA |
| | | | GCTTCGATCAACTACGCAGACAGGTACCAAAACAA |
| | | | ATGTTCTCGTCACGTGGGCATGAATCTGATGCTGTT |
| | | | TCCCTGCAGACAATGCGAGAGAATGAATCAGAATT |
| | | | CAAATATCTGCTTCACTCACGGACAGAAAGACTGT |
| | | | TTAGAGTGCTTTCCCGTGTCAGAATCTCAACCCGTT |
| | | | TCTGTCGTCAAAAAGGCGTATCAGAAACTGTGCTA |
| | | | CATTCATCATATCATGGGAAAGGTGCCAGACGCTT |
| | | | GCACTGCCTGCGATCTGGTCAATGTGGATTTGGAT |
| | | | GACTGCATCTTTGAACAATAAATGATTTAAATCAG |
| | | | GTatggctgccgatggttatcttccagattggctcgaggacactctctctgaaggaat |
| | | | aagacagtggtggaagctcaaacctggcccaccaccaccaaagcccgcagagcg |
| | | | gcataaggacgacagcaggggtcttgtgcttcctgggtacaagtacctcggaccctt |
| | | | caacggactcgacaagggagagccggtcaacgaggcagacgccgcggccctcg |
| | | | agcacgacaaagcctacgaccggcagctcgacagcggagacaacccgtacctca |
| | | | agtacaaccacgccgacgcggagtttcaggagcgccttaaagaagatacgtcttttg |
| | | | ggggcaacctcggacgagcagtcttccaggcgaaaaagagggttcttgaacctctg |
| | | | ggcctggttgaggaacctgttaagacggctccgggaaaaaagaggccggtagagc |
| | | | actctcctgtggagccagactcctcctcgggaaccggaaaggcgggccagcagcc |
| | | | tgcaagaaaaagattgaattttggtcagactggagacgcagactcagtacctgaccc |
| | | | ccagcctctcggacagccaccagcagccccctctggtctgggaactaatacgatgg |
| | | | ctacaggcagtggcgcaccaatggcagacaataacgagggcgccgacggagtgg |
| | | | gtaattcctcgggaaattggcattgcgattccacatggatgggcgacagagtcatcac |
| | | | caccagcacccgaacctgggccctgcccacctacaacaaccacctctacaaacaaa |
| | | | tttccagccaatcaggagcctcgaacgacaatcactactttggctacagcaccccttg |
| | | | ggggtattttgacttcaacagattccactgccacttttcaccacgtgactggcaaagac |
| | | | tcatcaacaacaactggggattccgacccaagagactcaacttcaagctctttaacatt |
| | | | caagtcaaagaggtcacgcagaatgacggtacgacgacgattgccaataaccttac |
| | | | cagcacggttcaggtgtttactgactcggagtaccagctcccgtacgtcctcggctcg |
| | | | gcgcatcaaggatgcctcccgccgttcccagcagacgtcttcatggtgccacagtat |
| | | | ggatacctcaccctgaacaacgggagtcaggcagtaggacgctcttcattttactgcc |
| | | | tggagtactttccttctcagatgctgcgtaccggaaacaactttaccttcagctacacttt |
| | | | tgaggacgttcctttccacagcagctacgctcacagccagagtctggaccgtctcatg |
| | | | aatcctctcatcgaccagtacctgtattacttgagcagaacaaacactccaagtggaa |
| | | | ccaccacgcagtcaaggcttcagttttctcaggccggagcgagtgacattcgggacc |
| | | | agtctaggaactggcttcctggaccctgttaccgccagcagcgagtatcaaagacat |
| | | | ctgcggataacaacaacagtgaatactcgtggactggagctaccaagtaccacctca |
| | | | atggcagagactctctggtgaatccgggcccggccatggcaagccacaaggacga |
| | | | tgaagaaaagttttttcctcagagcggggttctcatctttgggaagcaaggctcagag |
| | | | aaaacaaatgtggacattgaaaaggtcatgattacagacgaagaggaaatcaggac |
| | | | aaccaatcccgtggctacggagcagtatggttctgtatctaccaacctccagagagg |
| | | | caacagacaagcagctaccgcagatgtcaacacacaaggcgttcttccaggcatgg |
| | | | tctggcaggacagagatgtgtaccttcaggggcccatctgggcaaagattccacaca |
| | | | cggacggacattttcacccctctcccctcatgggggattcggacttaaacaccctcct |
| | | | ccacagattctcatcaagaacaccccggtacctgcgaatccttcgaccaccttcagtg |
| | | | cggcaaagtttgcttccttcatcacacagtactccacgggacaggtcagcgtggaga |
| | | | tcgagtgggagctgcagaaggaaaacagcaaacgctggaatcccgaaattcagta |
| | | | cacttccaactacaacaagtctgttaatgtggactttactgtggacactaatggcgtgta |
| | | | ttcagagcctcgccccattggcaccagatacctgactcgtaatctgtaaTTGCTT |
| | | | GTTAATCAATAAACCGTTTAATTCGTTTCAGTTGAA |
| | | | CTTTGGTCTCTGCCCTAGGGGAGGTCCTGTATTAGA |
| | | | GGTCACGTGAGTGTTTTGCGACATTTTGCGACACCA |
| | | | TGTGGTCACGCTGGGTATTTAAGCCCGAGTGAGCA |
| | | | CGCAGGGTCTCCATTTTGAAGCGGGAGGTTTGAAC |
| | | | ATACAGACGAAACTAGTTGGAGATTATCGAGCATT |
| | | | CTATCACGTCGACGACCACTAGTGAGTTACTAGAG |
| | | | CCGAGGGGTAACGTTGATGCTCCTAAAAATCTCTC |
| | | | GGTCGATGCAAGCGATTACACTCCTGTCACATCAT |
| | | | AATCGTTTGCTATTCAGGGCTTGATCAACACTGGAT |
| | | | TGCTTTTCACTTAAAGTATTATGCACGACAGGGTAC |
| | | | GTGTACCATATAAACCTGTTATAACTTACCTCAGAC |
| | | | TAGTTGGAAATGTGGCTAGATCTTAGCTTACGTTAC |
| | | | TAAAGGGTCCACGTTgaggtcctgtattagaggtcacgtgagtgttttg |
| | | | cgacattttgcgacaccatgtggtcacgctgggtatttaagcccgagtgagcacgca |
| | | | gggtctccattttgaaggggaggtttgaaATCCCAATGGCGCGCCG |
| | | | AGCTTGGCTCGAGCATGGTCATAGCTGTTTCCTGTG |
| | | | TGAAATTGTTATCCGCTCACAATTCCACACAACATA |
| | | | CGAGCCGGAAGCATAAAGTGTAAAGCCTGGGGTGC |
| | | | CTAATGAGTGAGCTAACTCACATTAATTGCGTTGC |
| | | | GCTCACTGCCCGCTTTCCAGTCGGGAAACCTGTCGT |
| | | | GCCAGCTGCATTAATGAATCGGCCAACGCGCGGGG |
| | | | AGAGGCGGTTTGCGTATTGGGCGCTCTTCCGCTTCC |
| | | | TCGCTCACTGACTCGCTGCGCTCGGTCGTTCGGCTG |
| | | | CGGCGAGCGGTATCAGCTCACTCAAAGGCGGTAAT |
| | | | ACGGTTATCCACAGAATCAGGGGATAACGCAGGAA |
| | | | AGAACATGTGAGCAAAAGGCCAGCAAAAGGCCAG |
| | | | GAACCGTAAAAAGGCCGCGTTGCTGGCGTTTTTCC |
| | | | ATAGGCTCCGCCCCCCTGACGAGCATCACAAAAAT |
| | | | CGACGCTCAAGTCAGAGGTGGCGAAACCCGACAG |
| | | | GACTATAAAGATACCAGGCGTTTCCCCCTGGAAGC |
| | | | TCCCTCGTGCGCTCTCCTGTTCCGACCCTGCCGCTT |
| | | | ACCGGATACCTGTCCGCCTTTCTCCCTTCGGGAAGC |
| | | | GTGGCGCTTTCTCATAGCTCACGCTGTAGGTATCTC |
| | | | AGTTCGGTGTAGGTCGTTCGCTCCAAGCTGGGCTGT |
| | | | GTGCACGAACCCCCCGTTCAGCCCGACCGCTGCGC |
| | | | CTTATCCGGTAACTATCGTCTTGAGTCCAACCCGGT |
| | | | AAGACACGACTTATCGCCACTGGCAGCAGCCACTG |
| | | | GTAACAGGATTAGCAGAGCGAGGTATGTAGGCGGT |
| | | | GCTACAGAGTTCTTGAAGTGGTGGCCTAACTACGG |
| | | | CTACACTAGAAGAACAGTATTTGGTATCTGCGCTCT |
| | | | GCTGAAGCCAGTTACCTTCGGAAAAAGAGTTGGTA |
| | | | GCTCTTGATCCGGCAAACAAACCACCGCTGGTAGC |
| | | | GGTGGTTTTTTTGTTTGCAAGCAGCAGATTACGCGC |
| | | | AGAAAAAAAGGATCTCAAGAAGATCCTTTGATCTT |
| | | | TTCTACGGGGTCTGACGCTCAGTGGAACGAAAACT |
| | | | CACGTTAAGGGATTTTGGTCATGAGATTATCAAAA |
| | | | AGGATCTTCACCTAGATCCTTTTAAATTAAAAATGA |
| | | | AGTTTTAAATCAATCTAAAGTATATATGAGTAAAC |
| | | | TTGGTCTGACAGTTAGAAAAACTCATCGAGCATCA |
| | | | AATGAAACTGCAATTTATTCATATCAGGATTATCA |
| | | | ATACCATATTTTTGAAAAAGCCGTTTCTGTAATGAA |
| | | | GGAGAAAACTCACCGAGGCAGTTCCATAGGATGGC |
| | | | AAGATCCTGGTATCGGTCTGCGATTCCGACTCGTCC |
| | | | AACATCAATACAACCTATTAATTTCCCCTCGTCAAA |
| | | | AATAAGGTTATCAAGTGAGAAATCACCATGAGTGA |
| | | | CGACTGAATCCGGTGAGAATGGCAAAAGTTTATGC |
| | | | ATTTCTTTCCAGACTTGTTCAACAGGCCAGCCATTA |
| | | | CGCTCGTCATCAAAATCACTCGCATCAACCAAACC |
| | | | GTTATTCATTCGTGATTGCGCCTGAGCGAGACGAA |
| | | | ATACGCGATCGCTGTTAAAAGGACAATTACAAACA |
| | | | GGAATCGAATGCAACCGGCGCAGGAACACTGCCA |
| | | | GCGCATCAACAATATTTTCACCTGAATCAGGATATT |
| | | | CTTCTAATACCTGGAATGCTGTTTTCCCAGGGATCG |
| | | | CAGTGGTGAGTAACCATGCATCATCAGGAGTACGG |
| | | | ATAAAATGCTTGATGGTCGGAAGAGGCATAAATTC |
| | | | CGTCAGCCAGTTTAGTCTGACCATCTCATCTGTAAC |
| | | | ATCATTGGCAACGCTACCTTTGCCATGTTTCAGAAA |
| | | | CAACTCTGGCGCATCGGGCTTCCCATACAATCGAT |
| | | | AGATTGTCGCACCTGATTGCCCGACATTATCGCGA |
| | | | GCCCATTTATACCCATATAAATCAGCATCCATGTTG |
| | | | GAATTTAATCGCGGCCTAGAGCAAGACGTTTCCCG |
| | | | TTGAATATGGCTCATACTCTTCCTTTTTCAATATTAT |
| | | | TGAAGCATTTATCAGGGTTATTGTCTCATGAGCGG |
| | | | ATACATATTTGAATGTATTTAGAAAAATAAACAAA |
| | | | TAGGGGTTCCGCGCACATTTCCCCGAAAAGTGCCA |
| | | | CCTGACGTCTAAGAAACCATTATTATCATGACATTA |
| | | | ACCTATAAAAATAGGCGTATCACGAGGCCCTTTCG |
| | | | TC |
| 74 | pYZZ463 | Rep2Cap2- | TCGCGCGTTTCGGTGATGACGGTGAAAACCTCTGA |
| | | p5p5p5 | CACATGCAGCTCCCGGAGACGGTCACAGCTTGTCT |
| | | | GTAAGCGGATGCCGGGAGCAGACAAGCCCGTCAG |
| | | | GGCGCGTCAGCGGGTGTTGGCGGGTGTCGGGGCTG |
| | | | GCTTAACTATGCGGCATCAGAGCAGATTGTACTGA |
| | | | GAGTGCACCATATGCGGTGTGAAATACCGCACAGA |
| | | | TGCGTAAGGAGAAAATACCGCATCAGGCGCCATTC |
| | | | GCCATTCAGGCTGCGCAACTGTTGGGAAGGGCGAT |
| | | | CGGTGCGGGCCTCTTCGCTATTACGCCAGCTGGCG |
| | | | AAAGGGGGATGTGCTGCAAGGCGATTAAGTTGGGT |
| | | | AACGCCAGGGTTTTCCCAGTCACGACGTTGTAAAA |
| | | | CGACGGCCAGTGAATTGACGCGTATTGGGATCGCG |
| | | | CAGCCGCCATGCCGGGGTTTTACGAGATTGTGATT |
| | | | AAGGTCCCCAGCGACCTTGACGAGCATCTGCCCGG |
| | | | CATTTCTGACAGCTTTGTGAACTGGGTGGCCGAGA |
| | | | AGGAATGGGAGTTGCCGCCAGATTCTGACATGGAT |
| | | | CTGAATCTGATTGAGCAGGCACCCCTGACCGTGGC |
| | | | CGAGAAGCTGCAGCGCGACTTTCTGACGGAATGGC |
| | | | GCCGTGTGAGTAAGGCCCCGGAGGCTCTTTTCTTTG |
| | | | TGCAATTTGAGAAGGGAGAGAGCTACTTCCACATG |
| | | | CACGTGCTCGTGGAAACCACCGGGGTGAAATCCAT |
| | | | GGTTTTGGGACGTTTCCTGAGTCAGATTCGCGAAA |
| | | | AACTGATTCAGAGAATTTACCGCGGGATCGAGCCG |
| | | | ACTTTGCCAAACTGGTTCGCGGTCACAAAGACCAG |
| | | | AAATGGCGCCGGAGGCGGGAACAAGGTGGTGGAT |
| | | | GAGTGCTACATCCCCAATTACTTGCTCCCCAAAACC |
| | | | CAGCCTGAGCTCCAGTGGGCGTGGACTAATATGGA |
| | | | ACAGTATTTAAGCGCCTGTTTGAATCTCACGGAGC |
| | | | GTAAACGGTTGGTGGCGCAGCATCTGACGCACGTG |
| | | | TCGCAGACGCAGGAGCAGAACAAAGAGAATCAGA |
| | | | ATCCCAATTCTGATGCGCCGGTGATCAGATCAAAA |
| | | | ACTTCAGCCAGGTACATGGAGCTGGTCGGGTGGCT |
| | | | CGTGGACAAGGGGATTACCTCGGAGAAGCAGTGG |
| | | | ATCCAGGAGGACCAGGCCTCATACATCTCCTTCAA |
| | | | TGCGGCCTCCAACTCGCGGTCCCAAATCAAGGCTG |
| | | | CCTTGGACAATGCGGGAAAGATTATGAGCCTGACT |
| | | | AAAACCGCCCCCGACTACCTGGTGGGCCAGCAGCC |
| | | | CGTGGAGGACATTTCCAGCAATCGGATTTATAAAA |
| | | | TTTTGGAACTAAACGGGTACGATCCCCAATATGCG |
| | | | GCTTCCGTCTTTCTGGGATGGGCCACGAAAAAGTT |
| | | | CGGCAAGAGGAACACCATCTGGCTGTTTGGGCCTG |
| | | | CAACTACCGGGAAGACCAACATCGCGGAGGCCATA |
| | | | GCCCACACTGTGCCCTTCTACGGGTGCGTAAACTG |
| | | | GACCAATGAGAACTTTCCCTTCAACGACTGTGTCG |
| | | | ACAAGATGGTGATCTGGTGGGAGGAGGGGAAGAT |
| | | | GACCGCCAAGGTCGTGGAGTCGGCCAAAGCCATTC |
| | | | TCGGAGGAAGCAAGGTGCGCGTGGACCAGAAATG |
| | | | CAAGTCCTCGGCCCAGATAGACCCGACTCCCGTGA |
| | | | TCGTCACCTCCAACACCAACATGTGCGCCGTGATT |
| | | | GACGGGAACTCAACGACCTTCGAACACCAGCAGCC |
| | | | GTTGCAAGACCGGATGTTCAAATTTGAACTCACCC |
| | | | GCCGTCTGGATCATGACTTTGGGAAGGTCACCAAG |
| | | | CAGGAAGTCAAAGACTTTTTCCGGTGGGCAAAGGA |
| | | | TCACGTGGTTGAGGTGGAGCATGAATTCTACGTCA |
| | | | AAAAGGGTGGAGCCAAGAAAAGACCCGCCCCCAG |
| | | | TGACGCAGATATAAGTGAGCCCAAACGGGTGCGCG |
| | | | AGTCAGTTGCGCAGCCATCGACGTCAGACGCGGAA |
| | | | GCTTCGATCAACTACGCAGACAGGTACCAAAACAA |
| | | | ATGTTCTCGTCACGTGGGCATGAATCTGATGCTGTT |
| | | | TCCCTGCAGACAATGCGAGAGAATGAATCAGAATT |
| | | | CAAATATCTGCTTCACTCACGGACAGAAAGACTGT |
| | | | TTAGAGTGCTTTCCCGTGTCAGAATCTCAACCCGTT |
| | | | TCTGTCGTCAAAAAGGCGTATCAGAAACTGTGCTA |
| | | | CATTCATCATATCATGGGAAAGGTGCCAGACGCTT |
| | | | GCACTGCCTGCGATCTGGTCAATGTGGATTTGGAT |
| | | | GACTGCATCTTTGAACAATAAATGATTTAAATCAG |
| | | | GTatggctgccgatggttatcttccagattggctcgaggacactctctctgaaggaat |
| | | | aagacagtggtggaagctcaaacctggcccaccaccaccaaagcccgcagagcg |
| | | | gcataaggacgacagcaggggtcttgtgcttcctgggtacaagtacctcggaccctt |
| | | | caacggactcgacaagggagagccggtcaacgaggcagacgccgcggccctcg |
| | | | agcacgacaaagcctacgaccggcagctcgacagcggagacaacccgtacctca |
| | | | agtacaaccacgccgacgcggagtttcaggagcgccttaaagaagatacgtcttttg |
| | | | ggggcaacctcggacgagcagtcttccaggcgaaaaagagggttcttgaacctctg |
| | | | ggcctggttgaggaacctgttaagacggctccgggaaaaaagaggccggtagagc |
| | | | actctcctgtggagccagactcctcctcgggaaccggaaagggggccagcagcc |
| | | | tgcaagaaaaagattgaattttggtcagactggagacgcagactcagtacctgaccc |
| | | | ccagcctctcggacagccaccagcagccccctctggtctgggaactaatacgatgg |
| | | | ctacaggcagtggcgcaccaatggcagacaataacgagggcgccgacggagtgg |
| | | | gtaattcctcgggaaattggcattgcgattccacatggatgggcgacagagtcatcac |
| | | | caccagcacccgaacctgggccctgcccacctacaacaaccacctctacaaacaaa |
| | | | tttccagccaatcaggagcctcgaacgacaatcactactttggctacagcaccccttg |
| | | | ggggtattttgacttcaacagattccactgccacttttcaccacgtgactggcaaagac |
| | | | tcatcaacaacaactggggattccgacccaagagactcaacttcaagctctttaacatt |
| | | | caagtcaaagaggtcacgcagaatgacggtacgacgacgattgccaataaccttac |
| | | | cagcacggttcaggtgtttactgactcggagtaccagctcccgtacgtcctcggctcg |
| | | | gcgcatcaaggatgcctcccgccgttcccagcagacgtcttcatggtgccacagtat |
| | | | ggatacctcaccctgaacaacgggagtcaggcagtaggacgctcttcattttactgcc |
| | | | tggagtactttccttctcagatgctgcgtaccggaaacaactttaccttcagctacacttt |
| | | | tgaggacgttcctttccacagcagctacgctcacagccagagtctggaccgtctcatg |
| | | | aatcctctcatcgaccagtacctgtattacttgagcagaacaaacactccaagtggaa |
| | | | ccaccacgcagtcaaggcttcagttttctcaggccggagcgagtgacattcgggacc |
| | | | agtctaggaactggcttcctggaccctgttaccgccagcagcgagtatcaaagacat |
| | | | ctgcggataacaacaacagtgaatactcgtggactggagctaccaagtaccacctca |
| | | | atggcagagactctctggtgaatccgggcccggccatggcaagccacaaggacga |
| | | | tgaagaaaagttttttcctcagagcggggttctcatctttgggaagcaaggctcagag |
| | | | aaaacaaatgtggacattgaaaaggtcatgattacagacgaagaggaaatcaggac |
| | | | aaccaatcccgtggctacggagcagtatggttctgtatctaccaacctccagagagg |
| | | | caacagacaagcagctaccgcagatgtcaacacacaaggcgttcttccaggcatgg |
| | | | tctggcaggacagagatgtgtaccttcaggggcccatctgggcaaagattccacaca |
| | | | cggacggacattttcacccctctcccctcatgggtggattcggacttaaacaccctcct |
| | | | ccacagattctcatcaagaacaccccggtacctgcgaatccttcgaccaccttcagtg |
| | | | cggcaaagtttgcttccttcatcacacagtactccacgggacaggtcagcgtggaga |
| | | | tcgagtgggagctgcagaaggaaaacagcaaacgctggaatcccgaaattcagta |
| | | | cacttccaactacaacaagtctgttaatgtggactttactgtggacactaatggcgtgta |
| | | | ttcagagcctcgccccattggcaccagatacctgactcgtaatctgtaaTTGCTT |
| | | | GTTAATCAATAAACCGTTTAATTCGTTTCAGTTGAA |
| | | | CTTTGGTCTCTGCCCTAGGGGAGGTCCTGTATTAGA |
| | | | GGTCACGTGAGTGTTTTGCGACATTTTGCGACACCA |
| | | | TGTGGTCACGCTGGGTATTTAAGCCCGAGTGAGCA |
| | | | CGCAGGGTCTCCATTTTGAAGCGGGAGGTTTGAAcg |
| | | | cgcagccgccaagccgtttcagttgaactttggtctctgcgtatttctttcttatctagtt |
| | | | tccatggaggtcctgtattagaggtcacgtgagtgttttgcgacattttgcgacaccatg |
| | | | tggtcacgctgggtatttaagcccgagtgagcacgcagggtctccattttgaagcgg |
| | | | gaggtttgaacgcgcagccgccaagccgtttcagttgaactttggtctctgcgtatttct |
| | | | ttcttatctagtttccatggaggtcctgtattagaggtcacgtgagtgttttgcgacattt |
| | | | tgcgacaccatgtggtcacgctgggtatttaagcccgagtgagcacgcagggtctcc |
| | | | attttgaagcgggaggtttgaaATCCCAATGGCGCGCCGAGCTT |
| | | | GGCTCGAGCATGGTCATAGCTGTTTCCTGTGTGAA |
| | | | ATTGTTATCCGCTCACAATTCCACACAACATACGA |
| | | | GCCGGAAGCATAAAGTGTAAAGCCTGGGGTGCCTA |
| | | | ATGAGTGAGCTAACTCACATTAATTGCGTTGCGCTC |
| | | | ACTGCCCGCTTTCCAGTCGGGAAACCTGTCGTGCC |
| | | | AGCTGCATTAATGAATCGGCCAACGCGCGGGGAGA |
| | | | GGCGGTTTGCGTATTGGGCGCTCTTCCGCTTCCTCG |
| | | | CTCACTGACTCGCTGCGCTCGGTCGTTCGGCTGCGG |
| | | | CGAGCGGTATCAGCTCACTCAAAGGCGGTAATACG |
| | | | GTTATCCACAGAATCAGGGGATAACGCAGGAAAG |
| | | | AACATGTGAGCAAAAGGCCAGCAAAAGGCCAGGA |
| | | | ACCGTAAAAAGGCCGCGTTGCTGGCGTTTTTCCAT |
| | | | AGGCTCCGCCCCCCTGACGAGCATCACAAAAATCG |
| | | | ACGCTCAAGTCAGAGGTGGCGAAACCCGACAGGA |
| | | | CTATAAAGATACCAGGCGTTTCCCCCTGGAAGCTC |
| | | | CCTCGTGCGCTCTCCTGTTCCGACCCTGCCGCTTAC |
| | | | CGGATACCTGTCCGCCTTTCTCCCTTCGGGAAGCGT |
| | | | GGCGCTTTCTCATAGCTCACGCTGTAGGTATCTCAG |
| | | | TTCGGTGTAGGTCGTTCGCTCCAAGCTGGGCTGTGT |
| | | | GCACGAACCCCCCGTTCAGCCCGACCGCTGCGCCT |
| | | | TATCCGGTAACTATCGTCTTGAGTCCAACCCGGTAA |
| | | | GACACGACTTATCGCCACTGGCAGCAGCCACTGGT |
| | | | AACAGGATTAGCAGAGCGAGGTATGTAGGCGGTGC |
| | | | TACAGAGTTCTTGAAGTGGTGGCCTAACTACGGCT |
| | | | ACACTAGAAGAACAGTATTTGGTATCTGCGCTCTG |
| | | | CTGAAGCCAGTTACCTTCGGAAAAAGAGTTGGTAG |
| | | | CTCTTGATCCGGCAAACAAACCACCGCTGGTAGCG |
| | | | GTGGTTTTTTTGTTTGCAAGCAGCAGATTACGCGCA |
| | | | GAAAAAAAGGATCTCAAGAAGATCCTTTGATCTTT |
| | | | TCTACGGGGTCTGACGCTCAGTGGAACGAAAACTC |
| | | | ACGTTAAGGGATTTTGGTCATGAGATTATCAAAAA |
| | | | GGATCTTCACCTAGATCCTTTTAAATTAAAAATGAA |
| | | | GTTTTAAATCAATCTAAAGTATATATGAGTAAACTT |
| | | | GGTCTGACAGTTAGAAAAACTCATCGAGCATCAAA |
| | | | TGAAACTGCAATTTATTCATATCAGGATTATCAATA |
| | | | CCATATTTTTGAAAAAGCCGTTTCTGTAATGAAGG |
| | | | AGAAAACTCACCGAGGCAGTTCCATAGGATGGCAA |
| | | | GATCCTGGTATCGGTCTGCGATTCCGACTCGTCCAA |
| | | | CATCAATACAACCTATTAATTTCCCCTCGTCAAAAA |
| | | | TAAGGTTATCAAGTGAGAAATCACCATGAGTGACG |
| | | | ACTGAATCCGGTGAGAATGGCAAAAGTTTATGCAT |
| | | | TTCTTTCCAGACTTGTTCAACAGGCCAGCCATTACG |
| | | | CTCGTCATCAAAATCACTCGCATCAACCAAACCGT |
| | | | TATTCATTCGTGATTGCGCCTGAGCGAGACGAAAT |
| | | | ACGCGATCGCTGTTAAAAGGACAATTACAAACAGG |
| | | | AATCGAATGCAACCGGCGCAGGAACACTGCCAGCG |
| | | | CATCAACAATATTTTCACCTGAATCAGGATATTCTT |
| | | | CTAATACCTGGAATGCTGTTTTCCCAGGGATCGCA |
| | | | GTGGTGAGTAACCATGCATCATCAGGAGTACGGAT |
| | | | AAAATGCTTGATGGTCGGAAGAGGCATAAATTCCG |
| | | | TCAGCCAGTTTAGTCTGACCATCTCATCTGTAACAT |
| | | | CATTGGCAACGCTACCTTTGCCATGTTTCAGAAACA |
| | | | ACTCTGGCGCATCGGGCTTCCCATACAATCGATAG |
| | | | ATTGTCGCACCTGATTGCCCGACATTATCGCGAGCC |
| | | | CATTTATACCCATATAAATCAGCATCCATGTTGGAA |
| | | | TTTAATCGCGGCCTAGAGCAAGACGTTTCCCGTTG |
| | | | AATATGGCTCATACTCTTCCTTTTTCAATATTATTG |
| | | | AAGCATTTATCAGGGTTATTGTCTCATGAGCGGAT |
| | | | ACATATTTGAATGTATTTAGAAAAATAAACAAATA |
| | | | GGGGTTCCGCGCACATTTCCCCGAAAAGTGCCACC |
| | | | TGACGTCTAAGAAACCATTATTATCATGACATTAA |
| | | | CCTATAAAAATAGGCGTATCACGAGGCCCTTTCGT |
| | | | C |

Production of rAAV

In some embodiments, a packaging plasmid of the present disclosure is useful in methods of producing rAAV In some embodiments, rAAV is produced by transfection of a host cell using a packaging plasmid as described herein In some embodiments, a host cell is a eukaryotic cell such as an insect-derived cell (Sf9), and/or mammalian cell such as a HEK293T, HEK293, Vero, or HeLA cell or any derivatives thereof, and/or prokaryotic cell.

In some embodiments, the present disclosure provides a packaging plasmid that is useful in producing increased titer of rAAV relative to a packaging plasmid not containing a p5 sequence downstream of the coding region. In some embodiments, the present disclosure provides a packaging plasmid that improves rAAV packaging, and/or the ratio of capsid containing DNA to empty capsid. In some embodiments, the present disclosure provides a packaging plasmid that improves rAAV genome integrity. In some embodiments, the present disclosure provides a packaging plasmid which results in reduced mis-packaged DNA impurities in rAAV.

In some embodiments, a method of producing a rAAV comprises transfection of a host cell with any packaging plasmid described herein and optionally with an adenoviral helper plasmid and/or a proviral plasmid. In some embodiments, a proviral plasmid comprises AAV inverted terminal repeats (ITRs) and a payload (e.g., transgene of interest).

In some embodiments, the present disclosure describes a packaging plasmid that reduces the manufacturing time and/or costs associated with production of rAAV in a host cell suspension, and/or adherent host cells and/or host cells grown in a bioreactor.

### Exemplification

The present invention encompasses the recognition of a problem with producing titers of AAV sufficiently high enough to produce large quantities of AAV derived cargo used as therapeutics to treat disease. The main purpose of the work described in this disclosure is to develop packaging plasmids that overcome the challenges commonly used packaging plasmids impose on producing high titers of rAAV.

### Example 1: Exemplary Packaging Plasmids

Exemplary plasmids as described herein include:

pYZZ233 (SEQ ID NO: 24) comprises one p5 promoter sequence (SEQ ID NO: 4) upstream of the coding region and two p5 promoter sequences (SEQ ID NO: 4), separated by a linker sequence (SEQ ID NO: 16), downstream of a coding region.

pYZZ235 (SEQ ID NO: 26) comprises two p5 sequences (SEQ ID NO: 4) separated by a linker sequence (SEQ ID NO: 16) downstream of a coding region.

Additional packaging plasmids were produced with the modifications described for pYZZ235, but with a unique linker sequence (SEQ ID NO: 17-23) for each additional plasmid, separating the two p5 sequences to create plasmids, pYZZ408, pYZZ409, pYZZ410, pYZZ411, pYZZ412, pYZZ413, pYZZ414 (SEQ ID NO: 28-34).

An additional packaging plasmid was produced with the same modifications described for pYZZ408, further comprising a second linker (SEQ ID NO: 17) separating a third p5 promoter sequence (SEQ ID NO: 4) downstream of the coding region, resulting in a plasmid named pYZZ415 (SEQ ID NO: 35).

### Example 2: Production of rAAV

The present example demonstrates that production of rAAV using packaging plasmids as described herein results in rAAV titer greater than rAAV titer when a wild-type packaging plasmid is used. Packaging plasmid candidates were designed and screened using suspension transient transfection in 24 deepwell plates. For transient transfection, cells were inoculated from the same shake flask culture into either deepwell (4.5 mL) or shake flasks (27 mL) at desired seeding densities on the transfection day. The proviral plasmid, the packaging plasmid (various), and the helper plasmid were transfected using Polyethylenimine (PEI) based method. 4 days post-transfection, cells were lysed. rAAVs in the lysates were quantified for AAV genome titers using digital droplet PCR (ddPCR).

HEK293 cells were transfected with packaging plasmids as indicated in FIG. 4, a helper plasmid, and a proviral plasmid. To compile and compare titer data across different transfection production experiments, a benchmark plasmid was used (BSG171, SEQ ID NO: 48). Relative titers from each experiment is calculated as Titer_{N}/ave (Titer_{BSG171}).

Candidate selection criteria: relative titer higher or comparable to the Benchmark (BSG171) and the current platform packaging plasmid (BSG098, SEQ ID NO: 47).

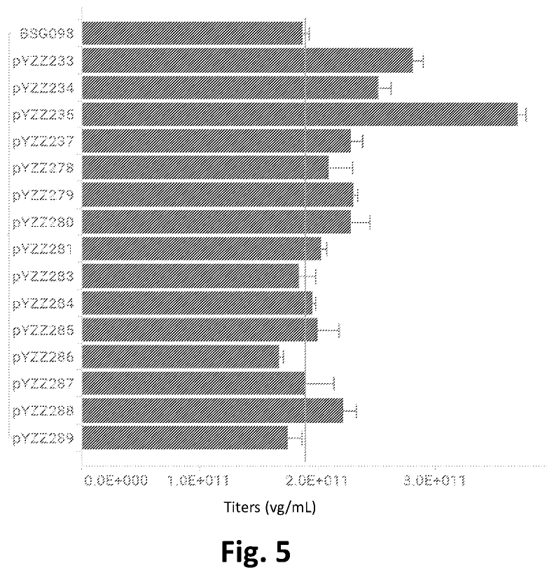

Additionally, HEK293 cells in deepwell cultures were transfected with a helper plasmid, a proviral plasmids, and packaging plasmids as indicated in FIG. 5. Transfection with packaging plasmids as described herein resulted in increased rAAV titer.

### Example 3: Packaging Plasmids Function in Various Host Cell Lines

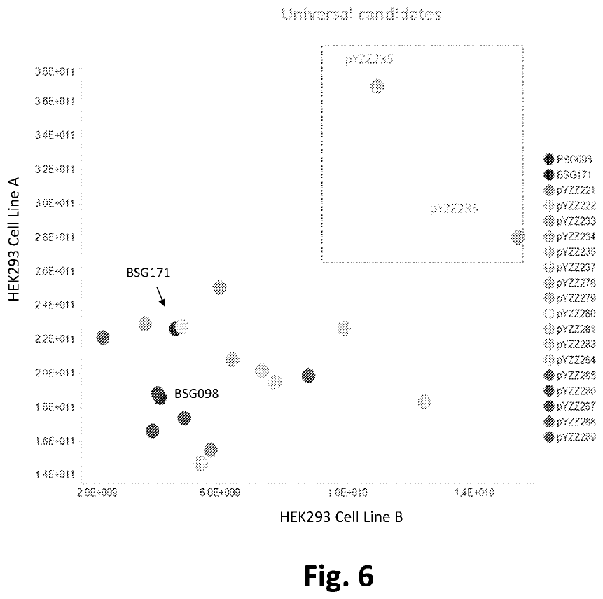

The present example demonstrates the universal nature of the packaging plasmids of the present disclosure. HEK293 Cell Line A and HEK293 Cell Line B host cells were independently transfected with each of BSG098, BSG171, pYZZ221, pYZZ222, pYZZ233, pYZZ234, pYZZ235, pYZZ237, pYZZ278, pYZZ279, pYZZ280, pYZZ281, pYZZ283, pYZZ284, pYZZ285, pYZZ286, pYZZ287, pYZZ288, and pYZZ289 plasmids. Titer of viral genomes resulting from each transfection was measured. Transfections using plasmids pYZZ235 and pYZZ233 were measured to yield the highest viral genome titer in both HEK293 Cell Line A and HEK293 Cell Line B host cells (see FIG. 6).

### Example 4: Additional Packaging Plasmids for Production of rAAV

The present example demonstrates that production of rAAV using packaging plasmids as described herein results in rAAV titer greater than rAAV titer when a wild-type packaging plasmid is used. Based on the plasmid design of top candidates from screening described in Example 3, additional candidates were designed.

HEK293 Cell Line A and HEK293 Cell Line B were seeded 2 days before transfection. The proviral plasmid, packaging plasmid, and the helper plasmid were transfected into the cells using PEI-mediated method. 4-days post transfection, the crude lysate containing rAAV particles were evaluated for genome titers by ddPCR.

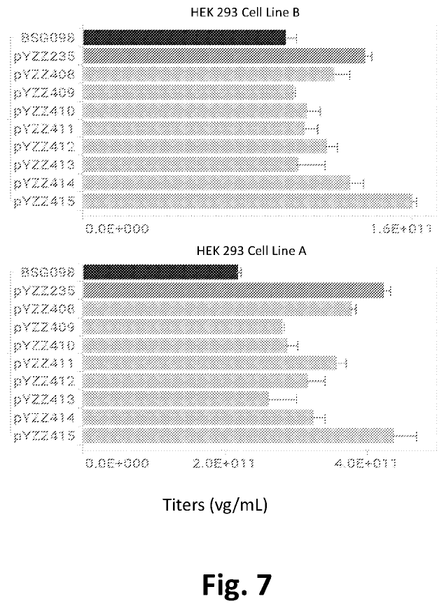

The iteration 2 packaging plasmid candidates outperformed or showed comparable titers to benchmark BSG098 (See FIG. 7).

### Example 5: Packaging Plasmids Useful for Production of rAAV in Bioreactors

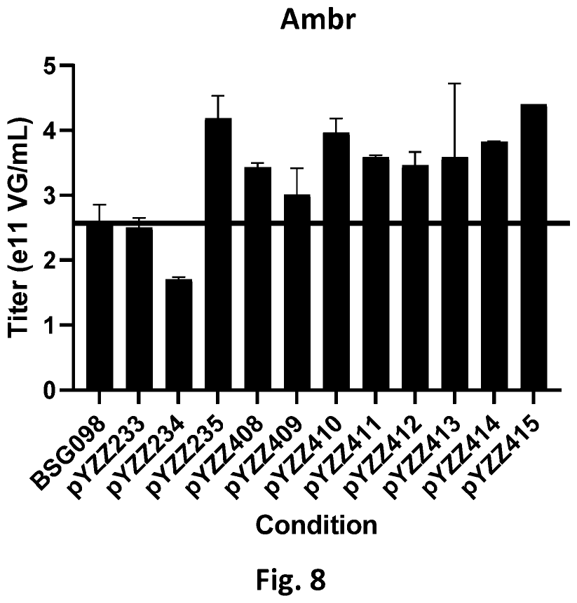

The present example demonstrates the packaging plasmids of the present disclosure are highly functional in production of rAAV in bioreactors. To evaluate the top candidates using a process that has more controls and is more relevant to the manufacturing process, suspension transient transfection was performed using AMBR mini-bioreactors. Cells from the same shake flask culture were used to inoculate an AMBR bioreactor. Inoculation and transfection occurred on the same day. The same lot of DNA and PEI were used. Cultures were harvested on day 4 post transfection. rAAV levels in the crude lysates were measured by ddPCR. Packaging plasmids as described herein produced higher titer compared to benchmark plasmids (see FIG. 8).

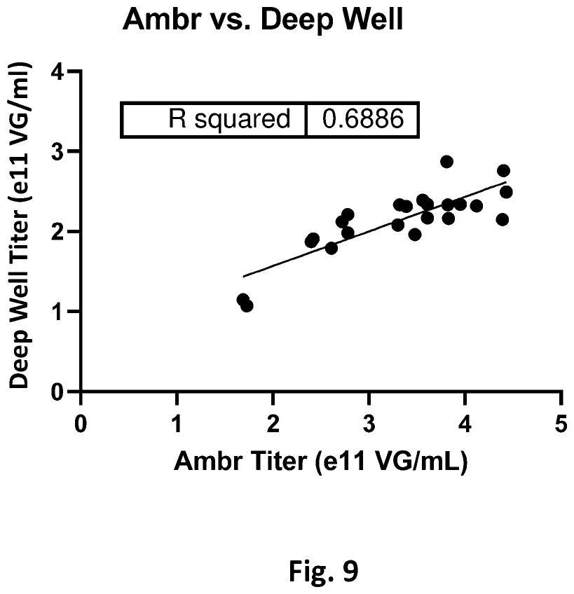

Deepwell cultures were transfected as described above. AMBR and deepwell cultures have similar titer trends (see FIG. 9).

### Example 6: Plasmids Useful for Production of rAAV in Bioreactors

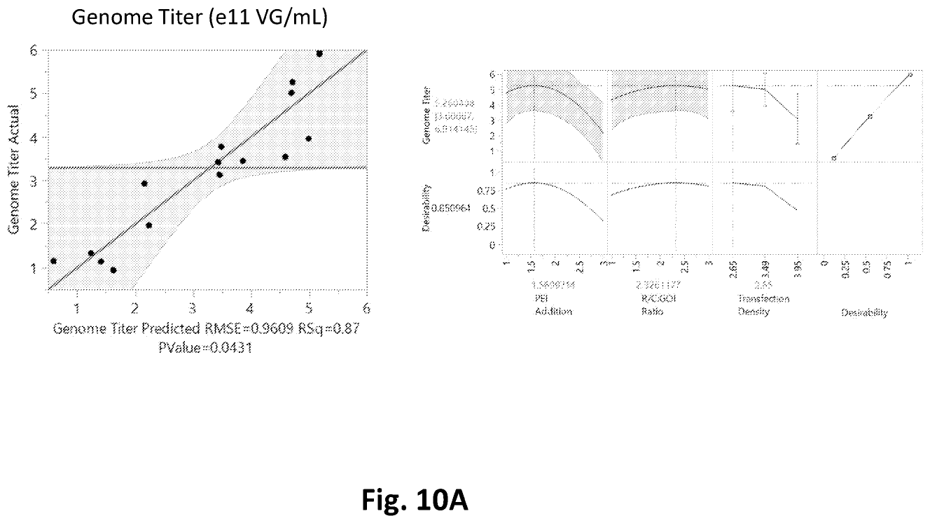

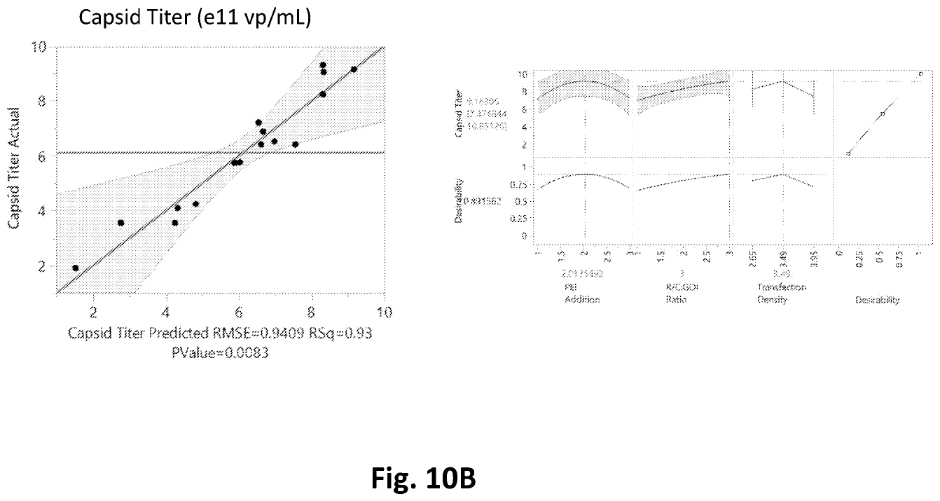

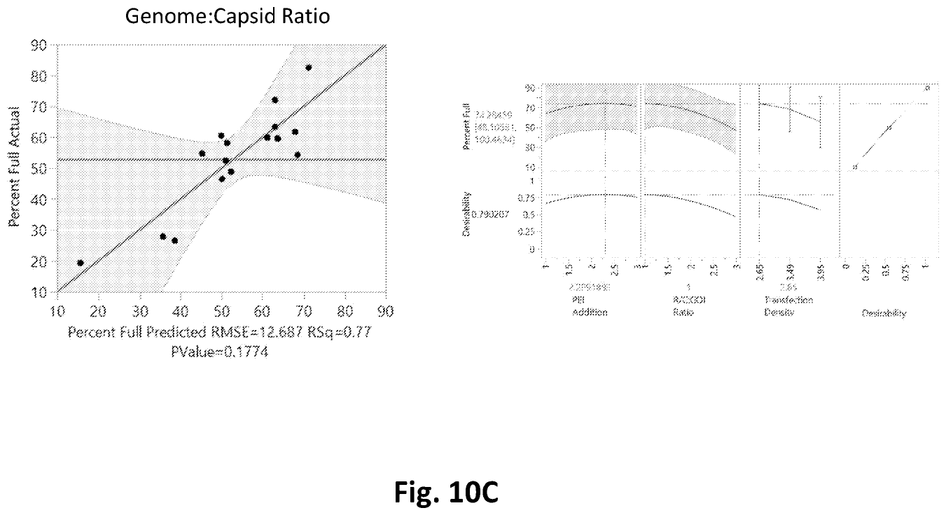

The present example further demonstrates plasmids of the present disclosure are highly functional in production of rAAV in bioreactors. Plasmid pYZZ410 was evaluated using the process described in Example 5. Genome titer, capsid titer, and percent full capsid (e.g., Genome: Capsid molar ratio, capsids containing genome) was measured by ddPCR. Genome titer, capsid titer, and percent full capsid measurements were made at various amounts of PEI addition (uL/mL) to a transfection reaction, transfection densities (vc/mL), and Rep/Cap: Gene of Interest (R/C: GOI) molar ratios. These measurements were used to determine the amount of PEI added to a transfection, R/C: GOI molar ratio, and transfection density that yielded the highest genome titer, capsid titer, and percent full capsids. Using these measurements, a linear regression equation was calculated to predict genome titer, capsid titer, and percent full capsid. Desirability scores that specify a condition (e.g., PEI addition, R/C: GOI molar ratio, transfection density) that achieve a desired optimization criteria (e.g., genome titer, capsid titer, percent full capsids) and were calculated using Jmp Statistical Discovery software. Transfections using plasmid pYZZ410 achieved an average genome titer of approximately 3.5×10E11 viral genome per milliliter (FIG. 10A). Viral genome titer was highest at a 1.56 μL/mL PEI, 2.32 R/C: GOI molar ratio, and 2.65 vc/mL, and achieved a desirability score of 0.85. Transfections using plasmid pYZZ410 achieved a 2.86-fold to 3.39-fold increase in capsid titer relative to a BSG098 control plasmid (FIG. 10B). Viral capsid titer was highest at 2.01 μL/mL PEI addition, 3.0 R/C: GOI molar ratio, and 3.49 vc/mL, and achieved a desirability score of 0.89. Transfections using plasmid pYZZ410 achieved an average percent full capsid (e.g., Genome: Capsid molar ratio, capsids containing genomes) of approximately 50% (FIG. 10C). Percentage of full capsids (e.g., containing genome) was highest at 2.29 μL/mL PEI addition, 1.0 R/C: GOI molar ratio, and 2.65 vc/mL, and achieved a desirability score of 0.79. A desirability score of 1.0 for a condition is considered the best score to achieve a criterion. Transfections were carried on HEK293 cells.

### Example 7: Plasmids Useful for Production of rAAV in Bioreactors

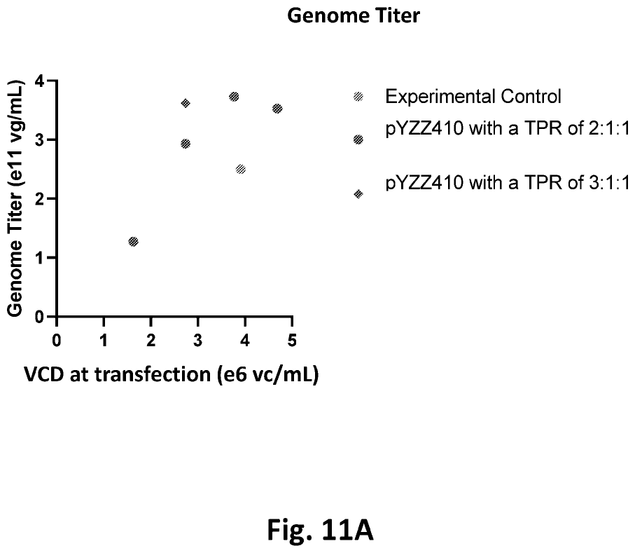

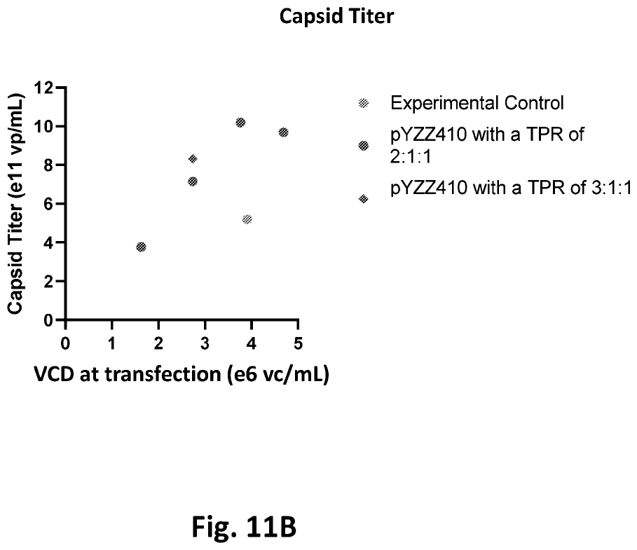

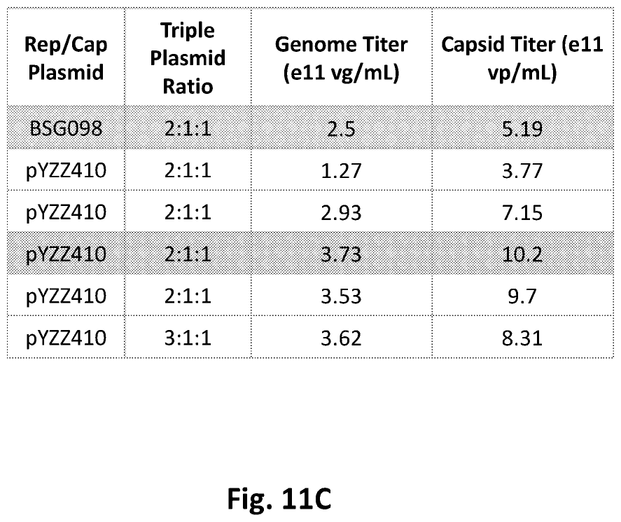

The present example further demonstrates plasmids of the present disclosure are highly functional in production of rAAV in bioreactors. To evaluate plasmid pYZZ410, suspension transient transfection was performed on host cells using 3 liter bioreactors. A cell culture was used to inoculate a bioreactor and then grown for 48 hours until a target biocapicitance transfection target was reached. Biocapacitance values were used to calculate viable cell density values (VCD) as viable cells per milliliter (vc/mL). Triple plasmid molar ratios of pYZZ410 plasmid to GOI to helper plasmid used for transfection were either 2:1:1 or 3:1:1. Transfections using a 2:1:1 triple plasmid molar ratio (TPR) were carried out at 4 VCD conditions ranging from 1E6 vc/mL to 5E6 vc/mL. Transfection using a 3:1:1 triple plasmid molar ratio was carried out using a single VCD condition. Plasmid BSG098 replaced pYZZ410 as an experimental control. Cultures were harvested on day 4 post transfection. Packaging plasmid pYZZ410 achieved higher rAAV genome titer (FIG. 11A and FIG. 11C) and rAAV capsid titer (FIG. 11B and FIG. 11C) relative to experimental control plasmid in all TPR molar ratios and VCD conditions except for the lowest VCD condition.

### Example 8: Plasmids Useful for Production of rAAV in Bioreactors

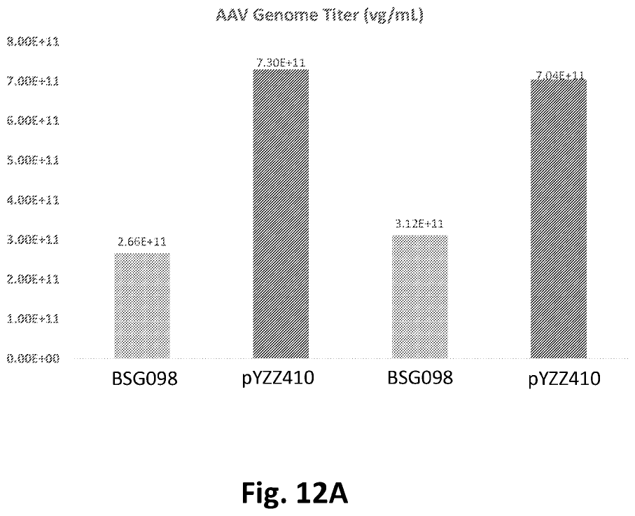

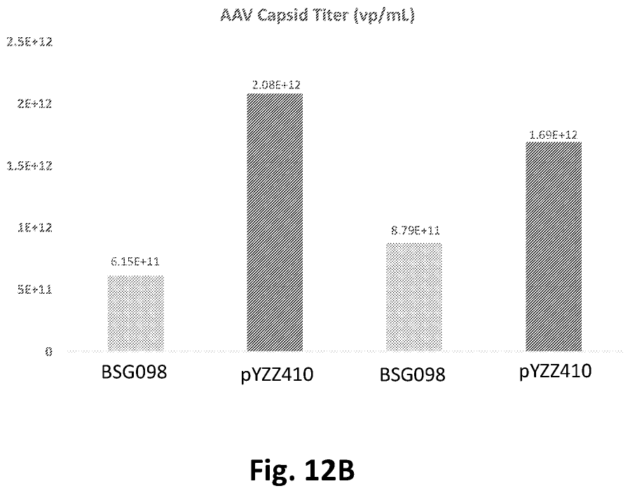

The present example further demonstrates plasmids of the present disclosure are highly functional in production of rAAV in bioreactors. Plasmid pYZZ410 was evaluated for rAAV genome titer and capsid titer as described in example 7, with the inclusion of certain additives. Plasmid pYZZ410 achieved 2.74-fold and 3.36-fold higher rAAV genome titer compared to control BSG098 plasmid (FIG. 12A). Plasmid PYZZ410 achieved a 1.92-fold and 2.25-fold higher rAAV capsid titer compared to control BSG098 plasmid (FIG. 12B). Results from two replicate experiments are depicted. Transfections were carried out on HEK293 cells.

### Example 9: Plasmids useful for Drug Substance Products

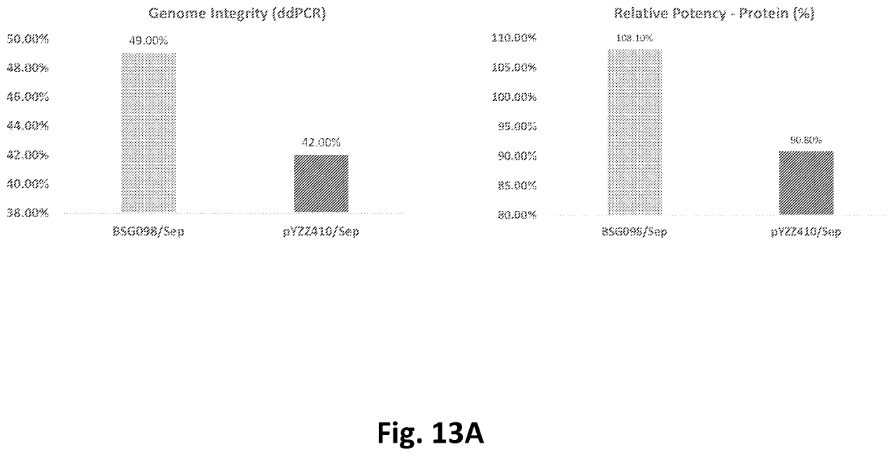

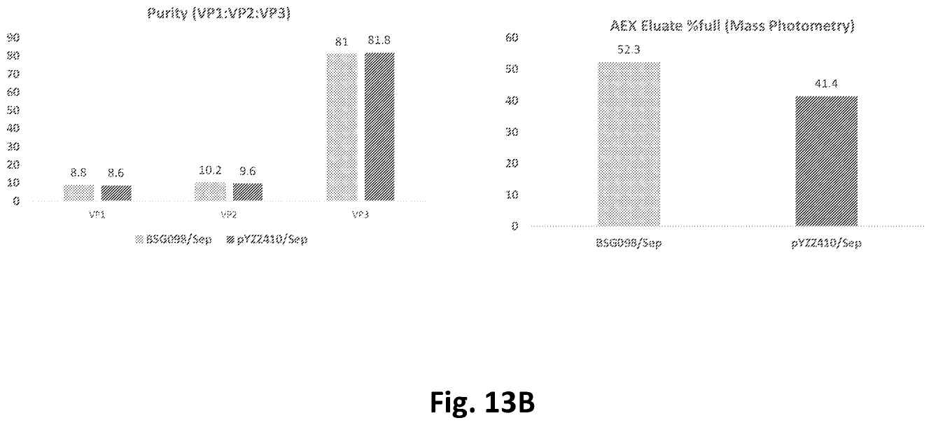

The present example demonstrates plasmids of the present disclosure achieve a drug substance product quality (e.g., genome integrity, relative potency, purity of VP1, VP2, and VP3 proteins, and percent full capsids) comparable to control BSG098 plasmid. Plasmid pYZZ410 was evaluated for rAAV genome integrity, relative potency of the drug substance produced by rAAV, purity of VP1, VP2, and VP3 proteins, and the percent of rAAV capsids that were full (e.g., contain a genome). Genome integrity was assessed using ddPCR and percent full capsids were assessed using anion exchange chromatography (AEX) coupled to mass photometry. Transfections using plasmid pYZZ410 achieved genome titer, drug substance relative potency, VP1, VP2, and VP3 purity, and percent full capsids that were comparable to control BSG098 plasmid (FIG. 13A-13B). Transfections were carried out using HEK293 cells.

Protein expression assays were conducted as in vitro cell-based assays whereby cells in 96-well plate format are transduced with the AAV product. The assay then uses immunochemistry to detect the amount of protein expressed from the AAV cargo in cells. The amount of protein expression is compared to the amount of protein expression in cells transduced with Reference Standard (AAV of the same type (e.g., AAV9) that has been qualified) and the value is reported as “% potency” in relation to the reference standard. In some embodiments, a 110% potency means 10% more protein expression occurred compared to the reference standard used.

### Example 10: Additional Plasmids for Production of rAAV

The present example further demonstrates plasmids of the present disclosure are highly functional in production of rAAV. Based on the plasmid design of top candidates from screening described in Example 3 and Example 4, additional candidates were designed using a different Capsid protein (e.g., Capsid 5). Packaging plasmid candidates were designed and screened using a suspension transient transfection. For transient transfection, cells were inoculated in a shake flask two days before transfection at a desired seeding density. 4 days post-transfection, cells were lysed. rAAVs in the lysates were quantified for rAAV genome titers using digital droplet PCR (ddPCR).

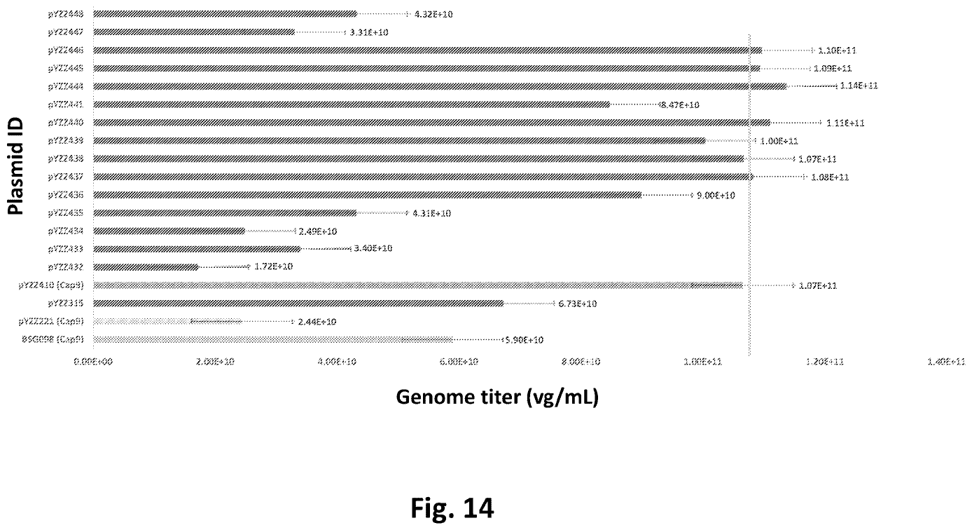

HEK293 cells were transfected with packaging plasmids, a helper plasmid, and a proviral plasmid. To compile and compare titer data across different transfection production experiments, a current platform packaging plasmid, BSG098, was used. Genome titers from each experiment were calculated as viral genome per milliliter culture (vg/mL). Transfection with packaging plasmids as described herein achieved a 2-fold increase in rAAV titer compared to plasmid BSG098 (FIG. 14).

### Example 11: Additional Plasmids for Production of rAAV

The present example further demonstrates plasmids of the present disclosure are highly functional in production of rAAV.

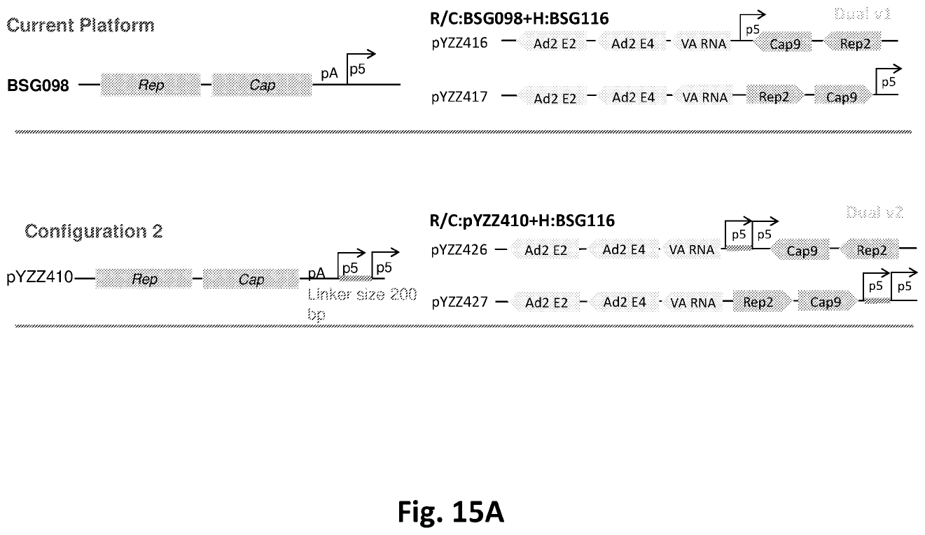

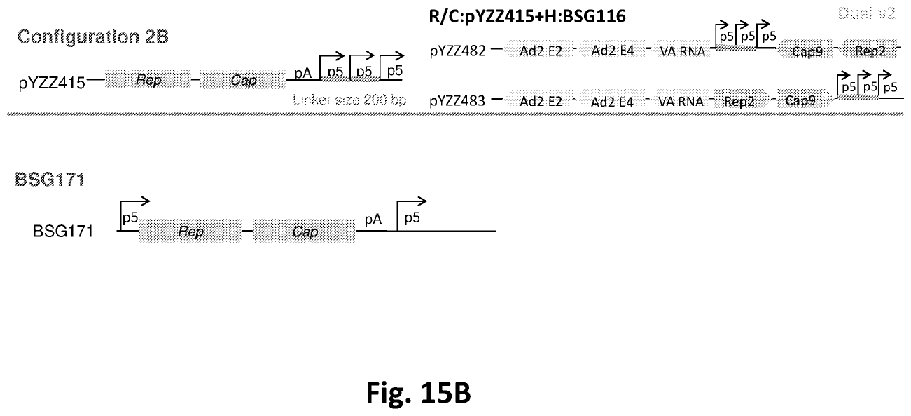

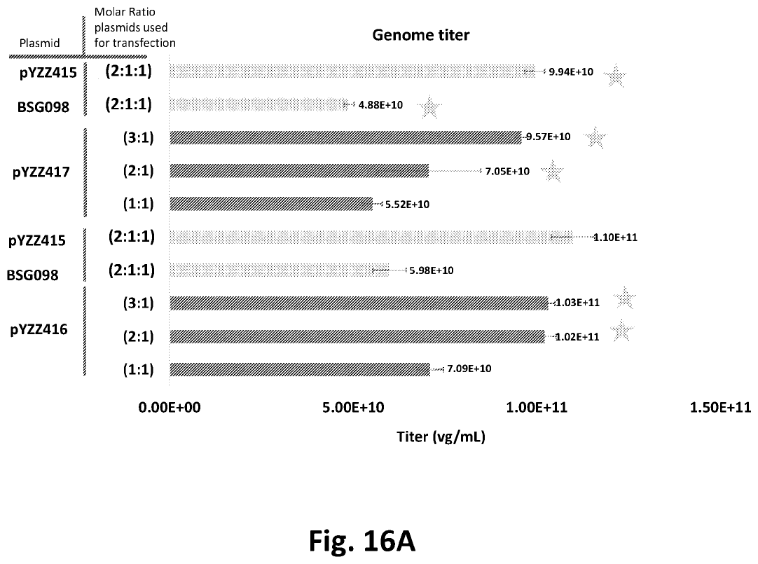

Additional plasmids were produced comprising p5 modifications described for BSG098 (e.g., “Current Platform”), pYZZ410 (e.g., “Configuration 2”), pYZZ415 (e.g., “Configuration 2B”), and BSG171, but with both packaging and adenoviral helper plasmids combined into a single packaging/helper plasmid used in a dual plasmid transfection with a proviral plasmid (e.g., gene of interest plasmid) (FIG. 15A-15B). Additional single packaging/helper plasmids pYZZ416 and pYZZ417 were modified like BSG098. Additional single packaging/helper plasmids pYZZ426 and pYZZ427 were modified like pYZZ410. Additional single packaging/helper plasmids pYZZ482 and pYZZ483 were modified like pYZZ415. Helper plasmid used to create single packaging/helper plasmids described herein were derived from plasmid BSG116. Single packaging/helper plasmids pYZZ416, pYZZ426, and pYZTT482 have a cis (e.g., same direction) orientation of packaging and helper plasmid elements. Single packaging/helper plasmids pYZZ417, pYZZ427, and pYZTT483 have a divergent (e.g., opposite direction) orientation of packaging and helper plasmid elements.

### Screening for Optimal Molar Ratio Using Single Packaging/Helper Plasmids

Additional plasmids were screened using suspension transient transfection in 24 deepwell plates. For transient transfection, cells were inoculated from the same shake flask culture into a deepwell (4.5 mL) at desired seeding densities on the transfection day. The proviral plasmid and the single packaging/helper plasmid were transfected using a Polyethylenimine (PEI) based method. 4 days post-transfection, cells were lysed and rAAVs in the lysates were quantified for rAAV genome titers using digital droplet PCR (ddPCR).

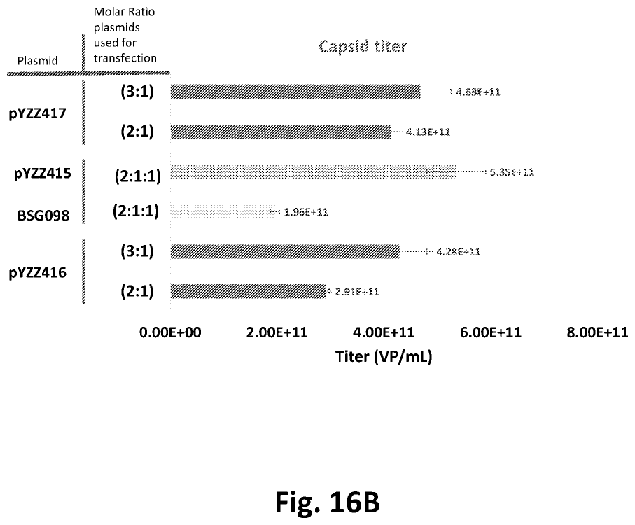

To determine an optimal molar ratio of packaging/helper plasmid to gene of interest (GOI) plasmid, dual plasmid transfections were carried out using single packaging/helper plasmid pYZZ417 or pYZZ416 at 3:1, 2:1, or 1:1 single packaging/helper plasmid to GOI plasmid molar ratios. Triple transfections using plasmid BSG098 or pYZZ415 were used as controls. Genome titer was calculated as Titer (vg/mL). Dual plasmid transfections using single packaging/helper plasmids pYZZ416 and pYZZ417 achieved better genome titer compared to triple plasmid transfection using BSG098 and comparable genome titer compared to triple plasmid transfection using pYZZ415 (FIG. 16A). Triple transfections molar ratios represent packaging plasmid to GOI plasmid to helper plasmid molar ratios.

Single packaging/helper plasmids pYZZ416 and pYZZ417 used in dual plasmid transfections at 2:1 or 3:1 molar ratios were analyzed further for capsid titer (VP/mL). Dual transfections using single packaging/helper plasmids pYZZ416 and pYZZ417 achieved better capsid titer relative to triple transfection using BSG098 and comparable genome titer to triple plasmid transfection using pYZZ415 (FIG. 16B).

### Example 12: Plasmids Useful for Production of rAAV

The present example further demonstrates plasmids of the present disclosure are highly functional in production of rAAV.

Additional single packaging/helper plasmids were used for dual transfections at a molar ratio of 3:1 as previously described. For comparison, triple transfections were performed as previously described using plasmids pYZZ415 and BSG098 at a molar ratio of 2:1:1 packaging plasmid to GOI plasmid to helper plasmid.

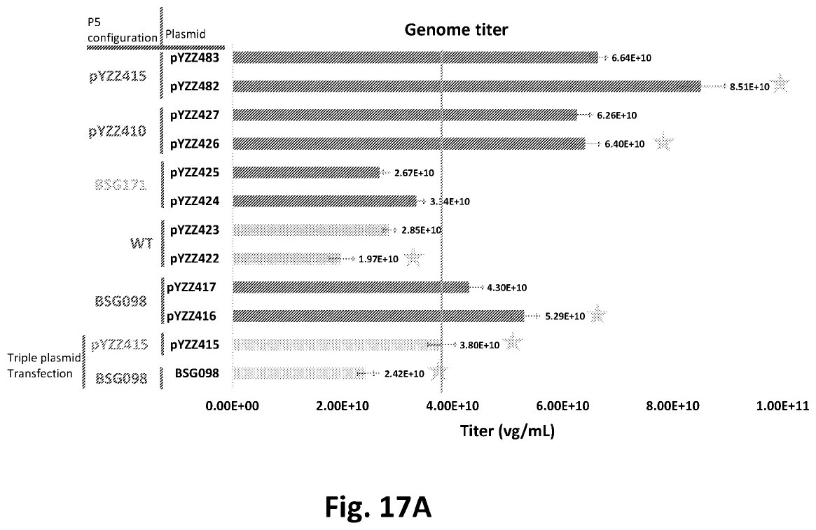

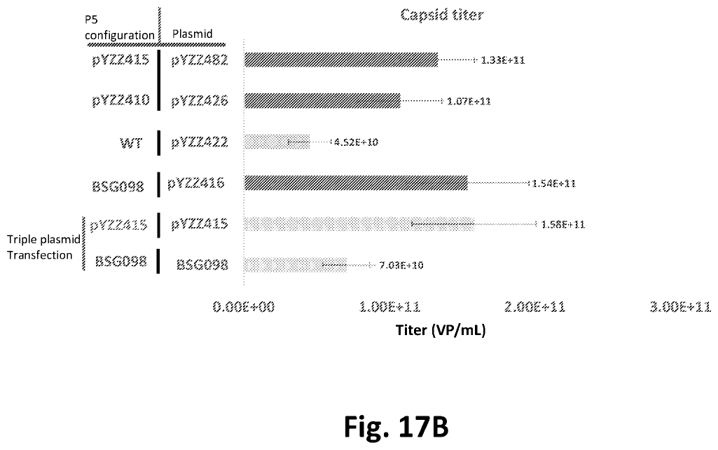

Additional single packaging/helper plasmids were produced with p5 modifications described for pYZZ415, pYZZ410, BSG171, WT, and BSG098 (FIG. 15A-15B). Additional single packaging/helper plasmids pYZZ483 and pYZZ482 were produced with p5 modifications described for pYZZ415. Additional single packaging/helper plasmids pYZZ427 and pYZZ426 were produced with p5 modifications described for pYZZ410. Additional single packaging/helper plasmids pYZZ425 and pYZZ424 were produced with p5 modifications described for BSG171. Additional single packaging/helper plasmids pYZZ423 and pYZZ422 were produced with a WT configuration. Additional single packaging/helper plasmid pYZZ417 and pYZZ416 were produced with p5 modifications described for BSG098. Dual plasmid transfections using single packaging/helper plasmid pYZZ482 achieved at least a 2.2-fold genome titer improvement over pYZZ415 and BSG098 plasmids used in triple plasmid transfections (FIG. 17A). Dual plasmid transfections using single packaging/helper plasmids pYZZ483, pYZZ426, and pYZZ427 achieved at least a 1.7-fold genome titer improvement over pYZZ415 and BSG171 plasmids used in triple plasmid transfections (FIG. 17A). Overall, dual plasmid transfections using single packaging/helper plasmids in Cis orientation resulted in higher titers than those in divergent orientation.

Single packaging/helper plasmids pYZZ482, pYZZ426, pYZZ422, and pYZZ416 were analyzed further for capsid titer (VP/mL). Plasmids pYZZ422, pYZZ415, and BS098 used in triple plasmid transfections were used as controls. Dual plasmid transfections using single packaging/helper plasmids pYZZ482, pYZZ426, and pYZZ416 achieved better or comparable capsid titer relative to pYZZ415 and BSG171 plasmids used in triple plasmid transfections and single packaging/helper plasmid pYZZ422 used in a dual plasmid transfection (FIG. 17B).

### Example 13: Plasmids Useful for Production of rAAV in Bioreactors

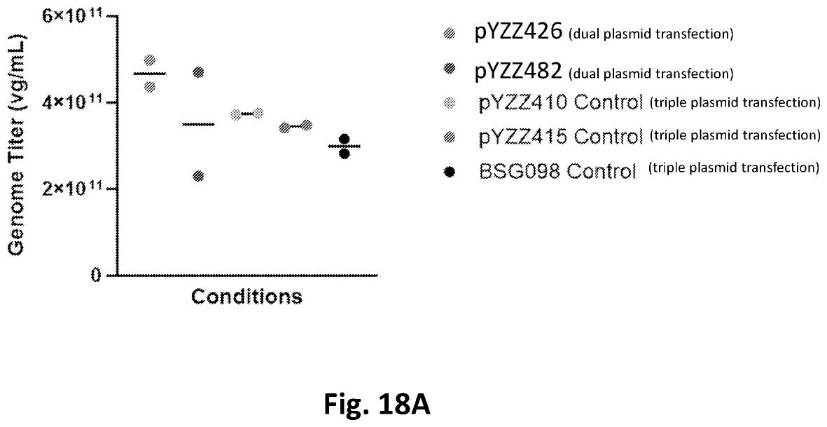

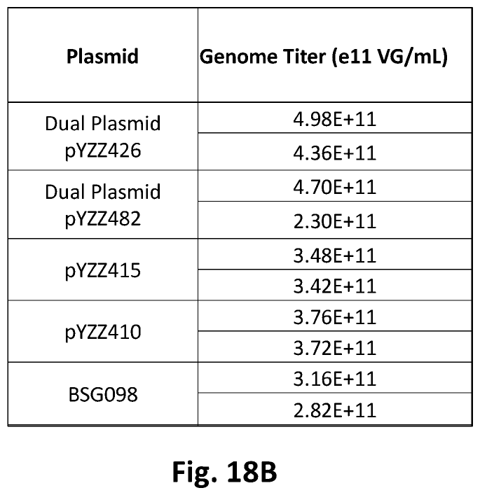

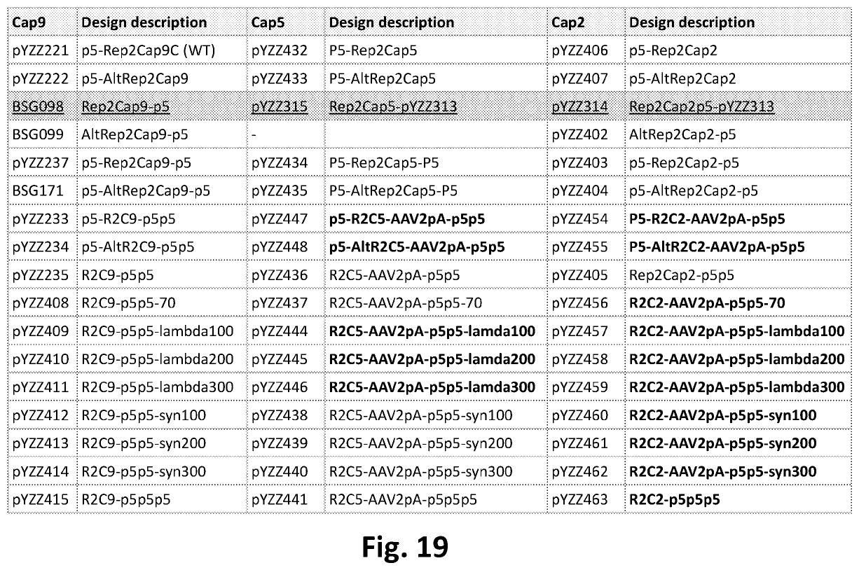

The present example further demonstrates plasmids of the present disclosure are highly functional in production of rAAV in bioreactors. Single packaging/helper plasmids pYZZ426 and pYZZ482 were evaluated for genome titer (vg/mL) using the process described in example 5. Triple plasmid transfections using plasmids pYZZ410, pYZZ415, and BSG098 were used as controls. Plasmid pYZZ426 outperformed all controls, and plasmid pYZZ482 outperformed all controls in one replicate (FIGS. 18A and 18B). All conditions were run in duplicates.

Single packaging/helper plasmids with p5 modifications described for BSG098 achieve improved titer compared to BSG098 and plasmids used (e.g., non single packaging/helper plasmids) in a triple transfection. Single packaging/helper plasmids with p5 modifications described for pYZZ410 and pYZZ415 achieve approximately 2-3 times improved titer compared to BSG098 and plasmids (e.g., non single packaging/helper plasmids) used in a triple transfection. Single packaging/helper plasmids with p5 modifications described herein achieve 1-3 times titer improvement compared to single plasmids that do not contain both packaging and helper plasmids (e.g., BSG098). Single packaging/helper plasmids were used in a dual plasmid transfection.

### Example 14: Plasmids for Production of rAAV

The present example demonstrates that rAAV genome titer can depend on the combination of AAV serotype and GOI used. Based on the plasmid design of top candidates from screening described in Example 3 and Example 4, additional candidates were tested using a Cap 2 protein and different genes of interest (e.g., GOI #1, GOI #2). GOI #1 was designed to be used with packaging plasmids comprising Cap9 (e.g., AAV9), while GOI #2 was designed to be used with packaging plasmids comprising Cap2 (e.g., AAV2). Packaging plasmid candidates were designed and screened using a suspension transient transfection. For transient transfection, cells were inoculated in a shake flask two days before transfection at a desired seeding density. 4 days post-transfection, cells were lysed. rAAVs in the lysates were quantified for rAAV genome titers using digital droplet PCR (ddPCR).

HEK293 TFS cells were transfected with packaging plasmids, a helper plasmid, and a proviral plasmid. To compile and compare titer data across different transfection production experiments, a current platform packaging plasmid, BSG098, was used. Genome titers from each experiment were calculated as viral genome per milliliter culture (vg/mL) using ddPCR.

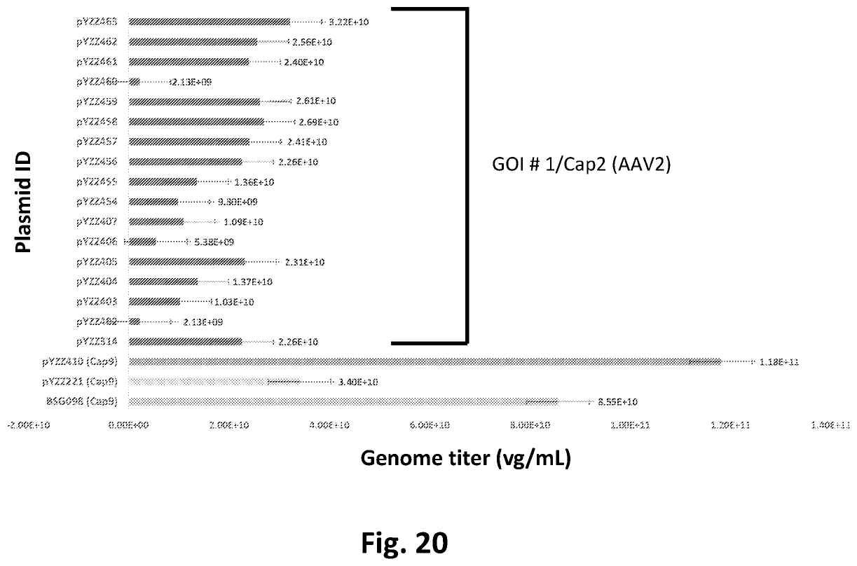

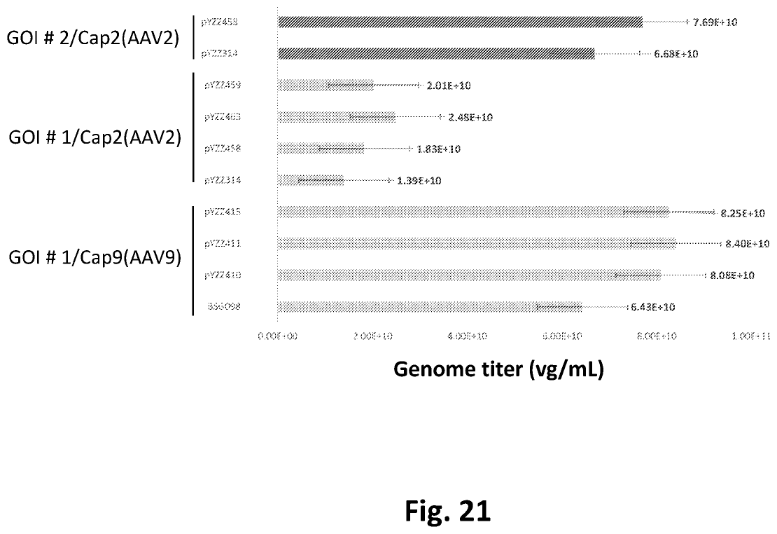

Results: Transfection with packaging plasmids comprising Cap2 (e.g., AAV2), and GOI #1 plasmid produced lower rAAV genome titer compared to the rAAV genome titer from transfection with packaging plasmids comprising Cap9 (e.g., AAV9), and GOI #1 plasmid (FIG. 20) demonstrating that GOI #1 produces higher yield rAAV titer when packaged in virus particles comprising Cap9. Transfection with packaging plasmids comprising Cap2 (e.g., AAV2), and GOI #2 plasmid achieved higher rAAV genome titer compared to the rAAV genome titer from transfection with packaging plasmids comprising Cap2 (e.g., AAV2), and GOI #1 (FIG. 21) demonstrating that GOI #2 produces higher yield rAAV titer when packaged in virus particles comprising Cap2. Transfection with packaging plasmids comprising Cap2 (e.g., AAV2), and GOI #2 plasmid achieved comparable rAAV genome titer as compared to packaging plasmids comprising Cap9 (e.g., AAV9), and GOI #1 (FIG. 21).

Without wishing to be bound by any particular theory, the data provided herein demonstrates that, rAAV yields can be modulated by using certain AAV serotypes with particular genes of interest. In some embodiments, packaging plasmids described herein can be designed with different capsid proteins to be useful for production of rAAV with different GOIs.

### EQUIVALENTS

Those skilled in the art will recognize, or be able to ascertain using no more than routine experimentation, many equivalents to the specific embodiments of the invention described herein. The scope of the present invention is not intended to be limited to the above Description, but rather is as set forth in the following claims:

We claim:

1. A packaging plasmid comprising a coding region and at least one p5 promoter sequence downstream of a coding region.

2. The packaging plasmid of claim 1, comprising one p5 promoter sequence downstream of the coding region.

3. The packaging plasmid of claim 1, comprising two p5 promoter sequences, a first p5 promoter sequence and a second p5 promoter sequence, downstream of the coding region.

4. The packaging plasmid of claim 3, further comprising a linker sequence between the first p5 promoter sequence and the second p5 promoter sequence.

5. The packaging plasmid of claim 1, comprising three p5 promoter sequences, a first p5 promoter sequence, a second p5 promoter sequence, and a third p5 promoter sequence, downstream of a coding region.

6. The packaging plasmid of claim 5, further comprising a linker sequence between the first p5 promoter sequence and the second p5 promoter sequence and a linker sequence between the second p5 promoter sequence and the third p5 promoter sequence.

7. The packaging plasmid of any one of the preceding claims, further comprising a pA sequence downstream of the coding region.

8. The packaging plasmid of claim 7, wherein the p5 promoter sequence is downstream of the pA sequence.

9. The packaging plasmid of claim 7 or 8, wherein the pA sequence is 80% identical to SEQ ID NO: 49.

10. The packaging plasmid of any one of the preceding claims, further comprising at least one p5 promoter sequence upstream of the coding region.

11. The packaging plasmid of claim 2, further comprising at least one p5 promoter sequence upstream of the coding region.

12. The packaging plasmid of claim 3, further comprising at least one p5 promoter sequence upstream of the coding region.

13. The packaging plasmid of any of the preceding claims wherein the p5 promoter sequence is at least 80% identical to any one of SEQ ID NOs: 4-15.

14. The packaging plasmid of any of the preceding claims, wherein the linker sequence is between approximately 1 bp-500 bp in length.

15. The packaging plasmid of any of the preceding claims, wherein the linker sequence comprises a nucleotide sequence that is at least 80% identical to any one of SEQ ID NOs: 16-23.

16. The packaging plasmid of any of the preceding claims, wherein the coding region comprises a Rep gene.

17. The packaging plasmid of any of the preceding claims, wherein the coding region comprises a Cap gene.

18. The packaging plasmid of claim 10, wherein the Rep gene nucleotide sequence is at least 80% identical to any one of SEQ ID NOs: 1-2.

19. The packaging plasmid of claim 17, wherein the Cap gene nucleotide sequence is at least 80% identical to SEQ ID NOs: 3, 50 or 51.

20. The packaging plasmid of claim 1, wherein the packaging plasmid comprises a nucleotide sequence at least 80% identical to any one of SEQ ID NOs: 24-46.

21. The packaging plasmid of any one of claims 1-20, further comprising one or more adenoviral helper function genes.

22. A method of producing a recombinant adeno-associated viral vector (rAAV) comprising transfecting a host cell with a packaging plasmid of any one of the preceding claims, optionally with a proviral plasmid and/or an adenoviral helper plasmid.

23. The method of claim 22, wherein titer of the rAAV is at least 2 fold higher than titer of rAAV produced using a packaging plasmid lacking a p5 promoter downstream of a coding region.
